(19) United States
(12) Patent Application Publication
(10) Pub. No.: US 2010/0189706 A1
(43) Pub. Date: Jul. 29, 2010

(54) ENZYMES FOR THE TREATMENT OF LIGNOCELLULOSICS, NUCLEIC ACIDS ENCODING THEM AND METHODS FOR MAKING AND USING THEM

(76) Inventors: Cathy Chang, San Marcos, CA (US); Justin T. Stege, San Diego, CA (US); Nahla Aboushadi, Katy, TX (US); Gordana Djordjevic, La Jolla, CA (US); Ellen Burke, San Diego, CA (US); Peter Luginbuhl, San Diego, CA (US); Mark Dycaico, San Diego, CA (US); Toby Richardson, San Diego, CA (US); John Poland, San Diego, CA (US); Ying Hefner, Encinitas, CA (US); Stacy Marie Miles, Chapel Hill, NC (US)

Correspondence Address: VERENIUM CORPORATION;Intellectual Property Department, P.O. Box 910550, SAN DIEGO, CA, 92191-0550, US

(21) Appl. No.: 12/525,303

(22) PCT Filed: Jan. 30, 2008
(86) PCT No.: PCT/US08/52517

§ 371 Date: Feb. 2, 2010

Related U.S. Application Data
(60) Provisional application No. 60/887,329, filed on Jan. 30, 2007.

(51) Int. Cl. A61K 38/44; A61K 38/46; C12N 11/16; A23L 1/28; C12P 21/06; C12P 19/34; C12P 7/04; C12P 7/16; C12P 7/06; C12N 11/14; C12N 11/00; C12N 11/08; C12N 9/00; C12N 9/02; C12N 9/10; C12N 9/42; C12N 5/10; C12N 1/21; C12N 1/15; C12N 1/19; C12N 15/63; C12P 1/00; D21C 3/00; C40B 40/06; C40B 40/10; C11D 7/42; C07K 16/00; C07H 21/04; A01K 67/00; A01H 5/00

(52) U.S. Cl. 424/94.4; 424/94.5; 424/94.6; 426/7; 426/61; 435/69.1; 435/91.2; 435/157; 435/160; 435/161; 435/176; 435/177; 435/180; 435/183; 435/189; 435/193; 435/209; 435/325; 435/348; 435/411; 435/412; 435/414; 435/415; 435/417; 435/419; 435/252.3; 435/254.11; 435/254.2; 435/320.1; 435/267; 435/278; 506/16; 506/18; 510/392; 530/387.9; 536/23.2; 536/24.3; 800/13; 800/298

(57) ABSTRACT

The invention provides polypeptides having a lignocellulolytic activity, e.g., a glycosyl hydrolase, a cellulase, an endoglucanase, a cellobiohydrolase, a beta-glucosidase, a xylanase, a mannanse, a xylosidase (e.g., a β-xylosidase), an arabinofuranosidase, and/or a glucose oxidase activity, polynucleotides encoding these polypeptides, and methods of making and using these polynucleotides and polypeptides. In one aspect, the invention provides polypeptides that can enzymatically process (hydrolyze) sugarcane bagasse, i.e., for sugarcane bagasse degradation, or for biomass processing, and polynucleotides encoding these enzymes, and making and using these polynucleotides and polypeptides. In one embodiment, the invention provides thermostable and thermotolerant forms of polypeptides of the invention. The polypeptides of the invention can be used in a variety of pharmaceutical, agricultural and industrial contexts; for example, the invention provides a multi-enzyme system that can hydrolyze polysaccharides in a bagasse component of sugarcane processed in sugar mills. The invention provides enzymes for the bioconversion of lignocellulosic residues into fermentable sugars; and these sugars can be used as a chemical feedstock for the production of ethanol and fuels, including biofuels such as bioethanol, biopropanol, biobutanol and biodiesels.

### REFERENCE TO SEQUENCE LISTING SUBMITTED VIA EFS-WEB

This application was filed electronically via the USPTO EFS-WEB server, as authorized and set forth in MPEP §1730 II.B.2.(a)(A), and this electronic filing includes an electronically submitted sequence (SEQ ID) listing; the entire content of this sequence listing is herein expressly incorporated by reference for all purposes. The sequence listing is identified on the electronically filed .txt file as follows:

| File Name | Date of Creation | Size (bytes) |
|---|---|---|
| 564462015440Seqlist.txt | January 28, 2008 | 2,530,632 bytes |

### FIELD OF THE INVENTION

This invention relates to molecular and cellular biology and biochemistry. In one aspect, the invention provides polypeptides having a lignocellulolytic (lignocellulosic) activity, e.g., a ligninolytic and cellulolytic activity, including, e.g., a glycosyl hydrolase, a cellulase, an endoglucanase, a cellobiohydrolase, a beta-glucosidase, a xylanase, a mannanse, a xylosidase (e.g., a β-xylosidase) and/or an arabinofuranosidase activity, polynucleotides encoding these polypeptides, and methods of making and using these polynucleotides and polypeptides. In one embodiment, the invention provides thermostable and thermotolerant forms of polypeptides of the invention. The polypeptides and nucleic acids of the invention are used in a variety of pharmaceutical, agricultural and industrial contexts; for example, as enzymes for the bioconversion of a biomass, e.g., lignocellulosic residues, into fermentable sugars, where in one aspect these sugars are used as a chemical feedstock for the production of ethanol and fuels, e.g., biofuels, e.g., synthetic liquid or gas fuels, including ethanol, methanol and the like.

### BACKGROUND

There is a great interest in the bioconversion of biomass, such as material comprising lignocellulosic residues, into fermentable sugars. These sugars can be used in turn as chemical feedstock for the production of a biofuel, which is a clean-burning renewable energy source. Accordingly, there is a need in the industry for non-chemical means for processing biomass to make clean-burning renewable fuels.

### SUMMARY

The invention provides polypeptides having lignocellulolytic (lignocellulosic) activity, e.g., a ligninolytic and cellulolytic activity, including, e.g., having cellulase, endoglucanase, cellobiohydrolase, β-glucosidase(beta-glucosidase), xylanase, xylosidase (e.g., β-xylosidase), and/or an arabinofuranosidase activity, and nucleic acids encoding them, and methods for making and using them. The invention provides enzymes for the bioconversion of any biomass, e.g., a lignocellulosic residue, into fermentable sugars or polysaccharides; and these sugars or polysaccharides can be used as a chemical feedstock for the production of alcohols such as ethanol, propanol, butanol and/or methanol, and in the production of fuels, e.g., biofuels such as synthetic liquids or gases, such as syngas.

In one aspect, the enzymes of the invention have an increased catalytic rate to improve the process of substrate (e.g., a lignocellulosic residue, cellulose, bagasse) hydrolysis. This increased efficiency in catalytic rate leads to an increased efficiency in producing sugars or polysaccharides, which can be useful in industrial, agricultural or medical applications, e.g., to make a biofuel or an alcohol such as ethanol, propanol, butanol and/or methanol. In one aspect, sugars produced by hydrolysis using enzymes of this invention can be used by microorganisms for alcohol (e.g., ethanol, propanol, butanol and/or methanol) production and/or fuel (e.g., biofuel) production.

In one aspect, the invention provides highly active polypeptides having lignocellulosic activity, e.g., polypeptides having an increased catalytic rate that include glycosyl hydrolases, endoglucanases, cellobiohydrolases, β-glucosidases(beta-glucosidases), xylanases, xylosidase (e.g., β-xylosidase) and/or arabinofuranosidases.

The invention provides industrial, agricultural or medical applications: e.g., biomass to biofuel, e.g., ethanol, propanol, butanol and/or methanol, using enzymes of the invention having decreased enzyme costs, e.g., decreased costs in biomass to biofuel conversion processes. Thus, the invention provides efficient processes for producing bioalcohols, biofuels and/or biofuel- (e.g., bioethanol-, propanol-, butanol- and/or methanol-) comprising compositions, including synthetic, liquid or gas fuels comprising a bioalcohol, from any biomass.

In one aspect, enzymes of the invention, including the enzyme “cocktails” of the invention (“cocktails” meaning mixtures of enzymes comprising at least one enzyme of this invention), are used to hydrolyze the major components of a lignocellulosic biomass, or any composition comprising cellulose and/or hemicellulose (lignocellulosic biomass also comprises lignin), e.g., seeds, grains, tubers, plant waste (such as a hay or straw, e.g., a rice straw or a wheat straw, or any the dry stalk of any cereal plant) or byproducts of food processing or industrial processing (e.g., stalks), corn (including cobs, stover, and the like), grasses (e.g., Indian grass, such as Sorghastrum nutans; or, switch grass, e.g., Panicum species, such as Panicum virgatum), wood (including wood chips, processing waste, such as wood waste), paper, pulp, recycled paper (e.g., newspaper); also including a monocot or a dicot, or a monocot corn, sugarcane or parts thereof (e.g., cane tops), rice, wheat, barley, switchgrass or Miscanthus; or a dicot oilseed crop, soy, canola, rapeseed, flax, cotton, palm oil, sugar beet, peanut, tree, poplar or lupine.

In one aspect, enzymes of the invention are used to hydrolyze cellulose comprising a linear chain of β-1,4-linked glucose moieties, and/or hemicellulose as a complex structure that varies from plant to plant. In one aspect, enzymes of the invention are used to hydrolyze hemicelluloses containing a backbone of β-1,4 linked xylose molecules with intermittent branches of arabinose, galactose, glucuronic acid and/or mannose. In one aspect, enzymes of the invention are used to hydrolyze hemicellulose containing non-carbohydrate constituents such as acetyl groups on xylose and ferulic acid esters on arabinose. In one aspect, enzymes of the invention are used to hydrolyze hemicelluloses covalently linked to lignin and/or coupled to other hemicellulose strands via diferulate crosslinks.

In one aspect, the compositions and methods of the invention are used in the enzymatic digestion of biomass and can comprise use of many different enzymes, including the cellulases and hemicellulases. Lignocellulosic enzymes used to practice the invention can digest cellulose to monomeric sugars, including glucose. In one aspect, compositions used to practice the invention can include mixtures of enzymes, e.g., glycosyl hydrolases, glucose oxidases, xylanases, xylosidases (e.g., β-xylosidases), cellobiohydrolases, and/or arabinofuranosidases or other enzymes that can digest hemicellulose to monomer sugars. Mixtures of the invention can comprise, or consist of, only enzymes of this invention, or can include at least one enzyme of this invention and another enzyme, which can also be a lignocellulosic enzyme and/or any other enzyme, e.g., a glucose oxidase.

In one aspect, compositions used to practice the invention include a “cellulase” that is a mixture of at least three different enzyme types, (1) endoglucanase, which cleaves internal β-1,4 linkages resulting in shorter glucooligosaccharides, (2) cellobiohydrolase, which acts in an “exo” manner processively releasing cellobiose units (β-1,4 glucose—glucose disaccharide), and (3) β-glucosidase, releasing glucose monomer from short cellooligosaccharides (e.g. cellobiose).

In one aspect, the enzymes of the invention have a glucanase, e.g., an endoglucanase, activity, e.g., catalyzing hydrolysis of internal endo-β-1,4- and/or β-1,3-glucanase linkages. In one aspect, the endoglucanase activity (e.g., endo-1,4-beta-D-glucan 4-glucano hydrolase activity) comprises hydrolysis of 1,4- and/or β-1,3-beta-D-glycosidic linkages in cellulose, cellulose derivatives (e.g., carboxy methyl cellulose and hydroxy ethyl cellulose) lichenin, beta-1,4 bonds in mixed beta-1,3 glucans, such as cereal beta-D-glucans or xyloglucans and other plant material containing cellulosic parts.

In one aspect, the enzymes of the invention have endoglucanase (e.g., endo-beta-1,4-glucanases, EC 3.2.1.4; endo-beta-1,3(1)-glucanases, EC 3.2.1.6; endo-beta-1,3-glucanases, EC 3.2.1.39) activity and can hydrolyze internal β-1,4- and/or β-1,3-glucosidic linkages in cellulose and glucan to produce smaller molecular weight glucose and glucose oligomers. The invention provides methods for producing smaller molecular weight glucose and glucose oligomers using these enzymes of the invention.

In one aspect, the enzymes of the invention are used to generate glucans, e.g., polysaccharides formed from 1,4-β- and/or 1,3-glycoside-linked D-glucopyranose. In one aspect, the endoglucanases of the invention are used in the food industry, e.g., for baking and fruit and vegetable processing, breakdown of agricultural waste, in the manufacture of animal feed, in pulp and paper production, textile manufacture and household and industrial cleaning agents. In one aspect, the enzymes, e.g., endoglucanases, of the invention are produced by a microorganism, e.g., by a fungi and/or a bacteria.

In one aspect, the enzymes, e.g., endoglucanases, of the invention are used to hydrolyze beta-glucans (β-glucans) which are major non-starch polysaccharides of cereals. The glucan content of a polysaccharide can vary significantly depending on variety and growth conditions. The physicochemical properties of this polysaccharide are such that it gives rise to viscous solutions or even gels under oxidative conditions. In addition glucans have high water-binding capacity. All of these characteristics present problems for several industries including brewing, baking, animal nutrition. In brewing applications, the presence of glucan results in wort filterability and haze formation issues. In baking applications (especially for cookies and crackers), glucans can create sticky doughs that are difficult to machine and reduce biscuit size. Thus, the enzymes, e.g., endoglucanases, of the invention are used to decrease the amount of β-glucan in a β-glucan-comprising composition, e.g., enzymes of the invention are used in processes to decrease the viscosity of solutions or gels; to decrease the water-binding capacity of a composition, e.g., a β-glucan-comprising composition; in brewing processes (e.g., to increase wort filterability and decrease haze formation), to decrease the stickiness of doughs, e.g., those for making cookies, breads, biscuits and the like.

In addition, carbohydrates (e.g., β-glucan) are implicated in rapid rehydration of baked products resulting in loss of crispiness and reduced shelf-life. Thus, the enzymes, e.g., endoglucanases, of the invention are used to retain crispiness, increase crispiness, or reduce the rate of loss of crispiness, and to increase the shelf-life of any carbohydrate-comprising food, feed or drink, e.g., a β-glucan-comprising food, feed or drink.

Enzymes, e.g., endoglucanases, of the invention are used to decrease the viscosity of gut contents (e.g., in animals, such as ruminant animals, or humans), e.g., those with cereal diets. Thus, in alternative aspects, enzymes, e.g., endoglucanases, of the invention are used to positively affect the digestibility of a food or feed and animal (e.g., human or domestic animal) growth rate, and in one aspect, are used to higher generate feed conversion efficiencies. For monogastric animal feed applications with cereal diets, beta-glucan is a contributing factor to viscosity of gut contents and thereby adversely affects the digestibility of the feed and animal growth rate. For ruminant animals, these beta-glucans represent substantial components of fiber intake and more complete digestion of glucans would facilitate higher feed conversion efficiencies. Accordingly, the invention provides animal feeds and foods comprising endoglucanases of the invention, and in one aspect, these enzymes are active in an animal digestive tract, e.g., in a stomach and/or intestine.

Enzymes, e.g., endoglucanases, of the invention are used to digest cellulose or any beta-1,4-linked glucan-comprising synthetic or natural material, including those found in any plant material. Enzymes, e.g., endoglucanases, of the invention are used as commercial enzymes to digest cellulose from any source, including all biological sources, such as plant biomasses, e.g., corn, grains, grasses (e.g., Indian grass, such as Sorghastrum nutans; or, switch grass, e.g., Panicum species, such as Panicum virgatum); also including a monocot or a dicot, or a monocot corn, sugarcane or parts thereof (e.g., cane tops), rice, wheat, barley, switchgrass or Miscanthus; or a dicot oilseed crop, soy, canola, rapeseed, flax, cotton, palm oil, sugar beet, peanut, tree, poplar or lupine; or, woods or wood processing byproducts, such as wood waste, e.g., in the wood processing, pulp and/or paper industry, in textile manufacture and in household and industrial cleaning agents, and/or in biomass waste processing.

In one aspect the invention provides compositions (e.g., pharmaceutical compositions, foods, feeds, drugs, dietary supplements) comprising the enzymes, polypeptides or polynucleotides of the invention. These compositions can be formulated in a variety of forms, e.g., as pills, capsules, tablets, gels, geltabs, lotions, pills, injectables, implants, liquids, sprays, powders, food, additives, supplements, feed or feed pellets, or as any type of encapsulated form, or any type of formulation.

The invention provides isolated, synthetic or recombinant nucleic acids comprising a nucleic acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity (homology) to an exemplary nucleic acid of the invention, including SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, SEQ ID NO:169, SEQ ID NO:171, SEQ ID NO:173, SEQ ID NO:175, SEQ ID NO:177, SEQ ID NO:179, SEQ ID NO:181, SEQ ID NO:183, SEQ ID NO:185, SEQ ID NO:187, SEQ ID NO:189, SEQ ID NO:191, SEQ ID NO:193, SEQ ID NO:195, SEQ ID NO:197, SEQ ID NO:199, SEQ ID NO:201, SEQ ID NO:203, SEQ ID NO:205, SEQ ID NO:207, SEQ ID NO:209, SEQ ID NO:211, SEQ ID NO:213, SEQ ID NO:215, SEQ ID NO:217, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:249, SEQ ID NO:251, SEQ ID NO:253, SEQ ID NO:255, SEQ ID NO:257, SEQ ID NO:259, SEQ ID NO:261, SEQ ID NO:263, SEQ ID NO:265, SEQ ID NO:267, SEQ ID NO:269, SEQ ID NO:271, SEQ ID NO:273, SEQ ID NO:275, SEQ ID NO:277, SEQ ID NO:279, SEQ ID NO:281, SEQ ID NO:283, SEQ ID NO:285, SEQ ID NO:287, SEQ ID NO:289, SEQ ID NO:291, SEQ ID NO:293, SEQ ID NO:295, SEQ ID NO:297, SEQ ID NO:299, SEQ ID NO:301, SEQ ID NO:303, SEQ ID NO:305, SEQ ID NO:307, SEQ ID NO:309, SEQ ID NO:311, SEQ ID NO:313, SEQ ID NO:315, SEQ ID NO:317, SEQ ID NO:319, SEQ ID NO:321, SEQ ID NO:323, SEQ ID NO:325, SEQ ID NO:327, SEQ ID NO:329, SEQ ID NO:331, SEQ ID NO:333, SEQ ID NO:335, SEQ ID NO:337, SEQ ID NO:339, SEQ ID NO:341, SEQ ID NO:343, SEQ ID NO:345, SEQ ID NO:347, SEQ ID NO:349, SEQ ID NO:351, SEQ ID NO:353, SEQ ID NO:355, SEQ ID NO:357, SEQ ID NO:359, SEQ ID NO:361, SEQ ID NO:363, SEQ ID NO:365, SEQ ID NO:367, SEQ ID NO:369, SEQ ID NO:370, SEQ ID NO:372, SEQ ID NO:373, SEQ ID NO:375, SEQ ID NO:376, SEQ ID NO:378, SEQ ID NO:379, SEQ ID NO:381, SEQ ID NO:382, SEQ ID NO:384, SEQ ID NO:385, SEQ ID NO:387, SEQ ID NO:388, SEQ ID NO:390, SEQ ID NO:391, SEQ ID NO:393, SEQ ID NO:394, SEQ ID NO:396, SEQ ID NO:397, SEQ ID NO:399, SEQ ID NO:400, SEQ ID NO:402, SEQ ID NO:403, SEQ ID NO:405, SEQ ID NO:406, SEQ ID NO:408, SEQ ID NO:409, SEQ ID NO:411, SEQ ID NO:412, SEQ ID NO:414, SEQ ID NO:415, SEQ ID NO:417, SEQ ID NO:418, SEQ ID NO:420, SEQ ID NO:421, SEQ ID NO:423, SEQ ID NO:425, SEQ ID NO:427, SEQ ID NO:429, SEQ ID NO:431, SEQ ID NO:433, SEQ ID NO: 435, SEQ ID NO:437, SEQ ID NO:439, SEQ ID NO:441, SEQ ID NO:443, SEQ ID NO:445, SEQ ID NO:447, SEQ ID NO:449, SEQ ID NO:451, SEQ ID NO:453, SEQ ID NO:455, SEQ ID NO:457, SEQ ID NO:459, SEQ ID NO:461, SEQ ID NO:463, SEQ ID NO:465, SEQ ID NO:467, SEQ ID NO:469 and/or SEQ ID NO:471, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, all the odd numbered SEQ ID NOs: between SEQ ID NO:489 and SEQ ID NO:700, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, and/or SEQ ID NO:720; which include both cDNA coding sequences and genomic (e.g., “gDNA”) sequences, and also including the sequences of Tables 1 to 4 (all of these sequences are “exemplary nucleic acids of the invention”), and the Examples, below (and these sequence are also set forth in the sequence listing), over a region of at least about 10, 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, or more residues; or over a region consisting of the protein coding region (e.g., the cDNA) or the genomic sequence; and all of these nucleic acid sequences, and the polypeptides they encode, encompass “sequences of the invention”.

In alternative aspects, these nucleic acids of the invention encode at least one polypeptide having a lignocellulolytic activity, e.g., a glycosyl hydrolase, cellulase, endoglucanase, cellobiohydrolase, β-glucosidase(beta-glucosidase), xylanase, xylosidase (e.g., β-xylosidase) and/or arabinofuranosidase activity. In alternative embodiments, a nucleic acid of the invention can encode a polypeptide capable of generating an antibody (or any binding fragment thereof) that can specifically bind to an exemplary polypeptide of the invention (listed below), or, these nucleic acids can be used as probes for identifying or isolating lignocellulotic enzyme-encoding nucleic acids, or to inhibit the expression of lignocellulotic enzyme-expressing nucleic acids (all these aspects referred to as the “nucleic acids of the invention”). In one aspect, the sequence identities are determined by analysis with a sequence comparison algorithm or by a visual inspection.

Nucleic acids of the invention also include isolated, synthetic or recombinant nucleic acids encoding an exemplary polypeptide (or peptide) of the invention which include polypeptides (e.g., enzymes) of the invention having the sequence of (or the subsequences of, or enzymatically active fragments of) SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:143, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:209, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, SEQ ID NO:260, SEQ ID NO:262, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, SEQ ID NO:360, SEQ ID NO:362, SEQ ID NO:364, SEQ ID NO:366, SEQ ID NO:368, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419, SEQ ID NO:422, SEQ ID NO:424, SEQ ID NO:426, SEQ ID NO:428, SEQ ID NO:430, SEQ ID NO:432, SEQ ID NO:434, SEQ ID NO: 436, SEQ ID NO:438, SEQ ID NO:440, SEQ ID NO:442, SEQ ID NO:444, SEQ ID NO:446, SEQ ID NO:448, SEQ ID NO:450, SEQ ID NO:452, SEQ ID NO:454, SEQ ID NO:456, SEQ ID NO:458, SEQ ID NO:460, SEQ ID NO:462, SEQ ID NO:464, SEQ ID NO:466, SEQ ID NO:468, SEQ ID NO:470 and/or SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, including sequences as set forth in Tables 1 to 4, and the sequences as set forth in the Sequence Listing (all of these sequences are “exemplary enzymes/polypeptides (or nucleic acids) of the invention”), and enzymatically active subsequences (fragments) thereof and/or immunologically active subsequences thereof (such as epitopes or immunogens) (all “peptides of the invention”) and variants thereof (all of these sequences encompassing polypeptide and peptide sequences of the invention).

In one embodiment, the polypeptide of the invention has a lignocellulosic activity, e.g., a glycosyl hydrolase, cellulase, endoglucanase, cellobiohydrolase, β-glucosidase(beta-glucosidase), xylanase, xylosidase (e.g., β-xylosidase) and/or an arabinofuranosidase activity.

In one aspect, the invention provides nucleic acids encoding lignocellulosic enzymes, e.g., glycosyl hydrolase, cellulase, endoglucanase, cellobiohydrolase, β-glucosidase(beta-glucosidase), xylanase, xylosidase (e.g., β-xylosidase), arabinofuranosidase, having a common novelty in that they are derived from mixed cultures. The invention provides cellulose or oligosaccharide hydrolyzing (degrading) enzyme-encoding nucleic acids isolated from mixed cultures comprising a polynucleotide of the invention, e.g., a sequence having at least about 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention, e.g., SEQ ID NO:1, SEQ ID NO:3, etc., through SEQ ID NO:471, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, all the odd numbered SEQ ID NOs: between SEQ ID NO:489 and SEQ ID NO:700, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, and/or SEQ ID NO:720 (see Tables 1 to 3, and the sequence listing), over a region of at least about 50, 75, 100, 150, 200, 250, 300, 350, 400, 450, 500, 550, 600, 650, 700, 750, 800, 850, 900, 950, 1000, 1050, 1100, 1150, or more, or over the full length of a coding sequence (e.g., a cDNA) or a genomic sequence (e.g., comprising exons and introns).

In one aspect, the invention provides nucleic acids encoding lignocellulosic enzymes, e.g., cellulase enzyme, endoglucanase enzyme, cellobiohydrolase enzyme, β-glucosidase enzyme (beta-glucosidase enzyme), xylanase enzyme, xylosidase (e.g., β-xylosidase) enzyme and/or an arabinofuranosidase enzyme-encoding; and/or glucose oxidase enzyme-encoding, nucleic acids, including exemplary polynucleotide sequences of the invention, see also Tables 1 to 4, and the Sequence Listing, and the polypeptides encoded by them, including enzymes of the invention, e.g., exemplary polypeptides of the invention, e.g., SEQ ID NO:2, SEQ ID NO:4, etc., through to SEQ ID NO:472 SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, (see Sequence Listing, and see also Tables 1 to 4), having a common novelty in that they are derived from a common source, e.g., an environmental source. Tables 2 and 3, below, indicate the initial source of some of the exemplary enzymes of the invention.

In one aspect, the invention also provides a lignocellulosic enzyme-encoding, e.g., a glycosyl hydrolase, an endoglucanase enzyme, cellobiohydrolase enzyme, β-glucosidase enzyme (beta-glucosidase enzyme), xylanase enzyme, xylosidase (e.g., β-xylosidase) and/or an arabinofuranosidase enzyme-encoding; and/or glucose oxidase enzyme-encoding, nucleic acids with a common novelty in that they are derived from environmental sources, e.g., mixed environmental sources.

In one aspect, the sequence comparison algorithm is a BLAST version 2.2.2 algorithm where a filtering setting is set to blastall -p blastp -d “nr pataa” -F F, and all other options are set to default.

Another aspect of the invention is an isolated, synthetic or recombinant nucleic acid including at least 10, 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, or more consecutive bases of a nucleic acid sequence of the invention, sequences substantially identical thereto, and the sequences complementary thereto.

In one aspect, the isolated, synthetic or recombinant nucleic acids of the invention encode a polypeptide having a lignocellulosic activity, e.g., a glycosyl hydrolase, cellulase, endoglucanase, cellobiohydrolase, β-glucosidase(beta-glucosidase), xylanase, xylosidase (e.g., β-xylosidase) and/or arabinofuranosidase activity; and/or glucose oxidase activity, which is thermostable. The polypeptide can retain a lignocellulosic activity under conditions comprising a temperature range of between about 37° C. to about 95° C.; between about 55° C. to about 85° C., between about 70° C. to about 95° C., or, between about 90° C. to about 95° C. The polypeptide can retain a lignocellulosic activity in temperatures in the range between about 1° C. to about 5° C., between about 5° C. to about 15° C., between about 15° C. to about 25° C., between about 25° C. to about 37° C., between about 37° C. to about 95° C., 96° C., 97° C., 98° C. or 99° C., between about 55° C. to about 85° C., between about 70° C. to about 75° C., or between about 90° C. to about 99° C., or 95° C., 96° C., 97° C., 98° C. or 99° C., or more.

In another aspect, the isolated, synthetic or recombinant nucleic acid encodes a polypeptide having a lignocellulosic activity, e.g., a glycosyl hydrolase, cellulase, endoglucanase, cellobiohydrolase, β-glucosidase(beta-glucosidase), xylanase, xylosidase (e.g., β-xylosidase) and/or arabinofuranosidase activity; and/or glucose oxidase activity, that can hydrolyze (degrade) soluble cellooligsaccharides and arabinoxylan oligomers into monomer xylose, arabinose and glucose, which is thermotolerant. The polypeptide can retain a lignocellulosic activity or glucose oxidase activity after exposure to a temperature in the range from greater than 37° C. to about 95° C. or anywhere in the range from greater than 55° C. to about 85° C. The polypeptide can retain a lignocellulosic activity after exposure to a temperature in the range between about 1° C. to about 5° C., between about 5° C. to about 15° C., between about 15° C. to about 25° C., between about 25° C. to about 37° C., between about 37° C. to about 95° C., 96° C., 97° C., 98° C. or 99° C., between about 55° C. to about 85° C., between about 70° C. to about 75° C., or between about 90° C. to about 95° C., or more. In one aspect, the polypeptide retains a lignocellulosic activity after exposure to a temperature in the range from greater than 90° C. to about 99° C., or 95° C., 96° C., 97° C., 98° C. or 99° C., at about pH 4.5, or more.

The invention provides isolated, synthetic or recombinant nucleic acids comprising a sequence that hybridizes under stringent conditions to a nucleic acid of the invention, including an exemplary nucleic acid sequence of the invention, e.g., the sequence of SEQ ID NO:1, SEQ ID NO:3, etc. through SEQ ID NO:471, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, all the odd numbered SEQ ID NOs: between SEQ ID NO:489 and SEQ ID NO:700, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, and/or SEQ ID NO:720 (see Tables 1 to 3, and the Sequence Listing), or fragments or subsequences thereof. In one aspect, the nucleic acid encodes a polypeptide having a lignocellulosic activity, e.g., a glycosyl hydrolase, cellulase, endoglucanase, β-glucosidase(beta-glucosidase), xylanase, xylosidase (e.g., β-xylosidase) and/or arabinofuranosidase activity, or can hydrolyze (degrade) soluble cellooligsaccharides and arabinoxylan oligomers into monomer xylose, arabinose and glucose. The nucleic acid can be at least about 10, 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200 or more residues in length or the full length of the gene or transcript (e.g., cDNA). In one aspect, the stringent conditions comprise a wash step comprising a wash in 0.2×SSC at a temperature of about 65° C. for about 15 minutes.

The invention provides a nucleic acid probe for identifying or isolating a nucleic acid encoding a polypeptide having a lignocellulosic activity, e.g., a glycosyl hydrolase, cellulase, endoglucanase, β-glucosidase(beta-glucosidase), xylanase, xylosidase (e.g., β-xylosidase) and/or arabinofuranosidase activity, or can hydrolyze (degrade) soluble cellooligsaccharides and arabinoxylan oligomers into monomer xylose, arabinose and glucose, wherein the probe comprises at least about 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000 or more, consecutive bases of a sequence comprising a sequence of the invention, or fragments or subsequences thereof, wherein the probe identifies the nucleic acid by binding or hybridization. The probe can comprise an oligonucleotide comprising at least about 10 to 50, about 20 to 60, about 30 to 70, about 40 to 80, or about 60 to 100 consecutive bases of a sequence comprising a sequence of the invention, or fragments or subsequences thereof.

The invention provides a nucleic acid probe for identifying or isolating a nucleic acid encoding a polypeptide having a lignocellulosic activity, e.g., a glycosyl hydrolase, cellulase, endoglucanase, β-glucosidase(beta-glucosidase), xylanase, xylosidase (e.g., β-xylosidase) and/or arabinofuranosidase activity, or can hydrolyze (degrade) soluble cellooligsaccharides and arabinoxylan oligomers into monomer xylose, arabinose and glucose, wherein the probe comprises a nucleic acid comprising a sequence at least about 10, 15, 20, 30, 40, 50, 60, 70, 80, 90, 100, 150, 200, 250, 300, 350, 400, 450, 500, 550, 600, 650, 700, 750, 800, 850, 900, 950, 1000 or more residues of a nucleic acid of the invention, e.g., a polynucleotide having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention. In one aspect, the sequence identities are determined by analysis with a sequence comparison algorithm or by visual inspection. In alternative aspects, the probe can comprise an oligonucleotide comprising at least about 10 to 50, about 20 to 60, about 30 to 70, about 40 to 80, or about 60 to 100 consecutive bases of a nucleic acid sequence of the invention, or a subsequence thereof.

The invention provides an amplification primer pair for amplifying (e.g., by PCR) a nucleic acid encoding a polypeptide having a lignocellulosic activity, e.g., a glycosyl hydrolase, cellulase, endoglucanase, β-glucosidase(beta-glucosidase), xylanase, xylosidase (e.g., β-xylosidase) and/or arabinofuranosidase activity, or can hydrolyze (degrade) soluble cellooligsaccharides and arabinoxylan oligomers into monomer xylose, arabinose and glucose, wherein the primer pair is capable of amplifying a nucleic acid comprising a sequence of the invention, or fragments or subsequences thereof. One or each member of the amplification primer sequence pair can comprise an oligonucleotide comprising at least about 10 to 50, or more, consecutive bases of the sequence, or about 10, 11, 12, 13, 14, 15, 16, 17, 18, 19, 20, 21, 22, 23, 24, 25, 26, 27, 28, 29, 30, 31, 32, 33, 34, 35, 36 or more consecutive bases of the sequence. The invention provides amplification primer pairs, wherein the primer pair comprises a first member having a sequence as set forth by about the first (the 5′) 12, 13, 14, 15, 16, 17, 18, 19, 20, 21, 22, 23, 24, 25, 26, 27, 28, 29, 30, 31, 32, 33, 34, 35, 36 or more residues of a nucleic acid of the invention, and a second member having a sequence as set forth by about the first (the 5′) 12, 13, 14, 15, 16, 17, 18, 19, 20, 21, 22, 23, 24, 25, 26, 27, 28, 29, 30, 31, 32, 33, 34, 35, 36 or more residues of the complementary strand of the first member.

The invention provides cellulase-encoding, e.g., endoglucanase, cellobiohydrolase, β-glucosidase(beta-glucosidase), xylanase, xylosidase (e.g., β-xylosidase), arabinofuranosidase, generated by amplification, e.g., polymerase chain reaction (PCR), using an amplification primer pair of the invention. The invention provides cellulase-encoding, e.g., endoglucanase, cellobiohydrolase, β-glucosidase(beta-glucosidase), xylanase, xylosidase (e.g., β-xylosidase), arabinofuranosidase, generated by amplification, e.g., polymerase chain reaction (PCR), using an amplification primer pair of the invention. The invention provides methods of making nucleic acid encoding an enzyme with lignocellulosic activity, e.g., a glycosyl hydrolase, cellulase, endoglucanase, cellobiohydrolase, β-glucosidase(beta-glucosidase), xylanase, xylosidase (e.g., β-xylosidase), arabinofuranosidase, by amplification, e.g., polymerase chain reaction (PCR), using an amplification primer pair of the invention. In one aspect, the amplification primer pair amplifies a nucleic acid from a library, e.g., a gene library, such as an environmental library.

The invention provides methods of amplifying a nucleic acid encoding a polypeptide having a lignocellulosic activity, e.g., a glycosyl hydrolase, cellulase, endoglucanase, cellobiohydrolase, β-glucosidase(beta-glucosidase), xylanase, xylosidase (e.g., β-xylosidase), arabinofuranosidase, or can hydrolyze (degrade) soluble cellooligsaccharides and arabinoxylan oligomers into monomer xylose, arabinose and glucose, comprising amplification of a template nucleic acid with an amplification primer sequence pair capable of amplifying a nucleic acid sequence of the invention, or fragments or subsequences thereof.

The invention provides expression cassettes comprising a nucleic acid of the invention or a subsequence thereof. In one aspect, the expression cassette can comprise the nucleic acid that is operably linked to a promoter. The promoter can be a viral, bacterial, mammalian or plant promoter. In one aspect, the plant promoter can be a potato, rice, corn, wheat, tobacco or barley promoter. The promoter can be a constitutive promoter. The constitutive promoter can comprise CaMV35S. In another aspect, the promoter can be an inducible promoter. In one aspect, the promoter can be a tissue-specific promoter or an environmentally regulated or a developmentally regulated promoter. Thus, the promoter can be, e.g., a seed-specific, a leaf-specific, a root-specific, a stem-specific or an abscission-induced promoter. In one aspect, a nucleic acid of the invention encoding an endogenous or heterologous signal sequence (see discussion, below) is expressed using an inducible promoter, an environmentally regulated or a developmentally regulated promoter, a tissue-specific promoter and the like. In alternative aspects, the promoter comprises a seed preferred promoter, such as e.g., the maize gamma zein promoter or the maize ADP-gpp promoter. In one aspect, the signal sequence targets the encoded protein of the invention to a vacuole, the endoplasmic reticulum, the chloroplast or a starch granule.

In one aspect, the expression cassette can further comprise a plant or plant virus expression vector. The invention provides cloning vehicles comprising an expression cassette (e.g., a vector) of the invention or a nucleic acid of the invention. The cloning vehicle can be a viral vector, a plasmid, a phage, a phagemid, a cosmid, a fosmid, a bacteriophage or an artificial chromosome. The viral vector can comprise an adenovirus vector, a retroviral vector or an adeno-associated viral vector. The cloning vehicle can comprise a bacterial artificial chromosome (BAC), a plasmid, a bacteriophage P1-derived vector (PAC), a yeast artificial chromosome (YAC), or a mammalian artificial chromosome (MAC).

The invention provides transformed cells comprising a nucleic acid of the invention or an expression cassette (e.g., a vector, plasmid, etc.) of the invention, or a cloning vehicle (e.g., artificial chromosome) of the invention. In one aspect, the transformed cell can be a bacterial cell, a mammalian cell, a fungal cell, a yeast cell, an insect cell or a plant cell. In one aspect, the plant cell can be soybeans, rapeseed, oilseed, tomato, cane sugar, a cereal, a potato, wheat, rice, corn, tobacco or barley cell; the plant cell also can be a monocot or a dicot, or a monocot corn, sugarcane, rice, wheat, barley, Indian grass, switchgrass or Miscanthus; or a dicot oilseed crop, soy, canola, rapeseed, flax, cotton, palm oil, sugar beet, peanut, tree, poplar or lupine.

The invention provides transgenic non-human animals comprising a nucleic acid of the invention or an expression cassette (e.g., a vector) of the invention. In one aspect, the animal is a mouse, a cow, a rat, a pig, a goat or a sheep.

The invention provides transgenic plants comprising a nucleic acid of the invention or an expression cassette (e.g., a vector) of the invention. The transgenic plant can be any cereal plant, a corn plant, a potato plant, a tomato plant, a wheat plant, an oilseed plant, a rapeseed plant, a soybean plant, a rice plant, a barley plant or a tobacco plant. The transgenic plant can be a monocot or a dicot, or a monocot corn, sugarcane, rice, wheat, barley, switchgrass or Miscanthus; or a dicot oilseed crop, soy, canola, rapeseed, flax, cotton, palm oil, sugar beet, peanut, tree, poplar or lupine.

The invention provides transgenic seeds comprising a nucleic acid of the invention or an expression cassette (e.g., a vector) of the invention. The transgenic seed can be a cereal plant, a corn seed, a wheat kernel, an oilseed, a rapeseed, a soybean seed, a palm kernel, a sunflower seed, a sesame seed, a peanut or a tobacco plant seed. The transgenic seed can be derived from a monocot or a dicot, or a monocot corn, sugarcane, rice, wheat, barley, switchgrass or Miscanthus; or a dicot oilseed crop, soy, canola, rapeseed, flax, cotton, palm oil, sugar beet, peanut, tree, poplar or lupine.

The invention provides an antisense oligonucleotide comprising a nucleic acid sequence complementary to or capable of hybridizing under stringent conditions to a nucleic acid of the invention. The invention provides methods of inhibiting the translation of a lignocellulosic enzyme, e.g., a glycosyl hydrolase, cellulase, endoglucanase, cellobiohydrolase, mannanase, β-glucosidase(beta-glucosidase), xylanase, xylosidase (e.g., β-xylosidase) and/or arabinofuranosidase enzyme message in a cell comprising administering to the cell or expressing in the cell an antisense oligonucleotide comprising a nucleic acid sequence complementary to or capable of hybridizing under stringent conditions to a nucleic acid of the invention. In one aspect, the antisense oligonucleotide is between about 10 to 50, about 20 to 60, about 30 to 70, about 40 to 80, or about 60 to 100 bases in length, e.g., 10, 15, 20, 25, 30, 35, 40, 45, 50, 55, 60, 65, 70, 75, 80, 85, 90, 95, 100 or more bases in length. The invention provides methods of inhibiting the translation of a lignocellulosic enzyme message in a cell comprising administering to the cell or expressing in the cell an antisense oligonucleotide comprising a nucleic acid sequence complementary to or capable of hybridizing under stringent conditions to a nucleic acid of the invention.

The invention provides double-stranded inhibitory RNA (RNAi, or RNA interference) molecules (including small interfering RNA, or siRNAs, for inhibiting transcription, and microRNAs, or miRNAs, for inhibiting translation) comprising a subsequence of a sequence of the invention. In one aspect, the siRNA is between about 21 to 24 residues, or, about at least 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100 or more duplex nucleotides in length. The invention provides methods of inhibiting the expression of a lignocellulosic enzyme, e.g., a glycosyl hydrolase, cellulase, endoglucanase, cellobiohydrolase, β-glucosidase(beta-glucosidase), xylanase, xylosidase (e.g., β-xylosidase) and/or arabinofuranosidase activity, e.g., can hydrolyze (degrade) soluble cellooligsaccharides and arabinoxylan oligomers into monomer xylose, arabinose and glucose, in a cell comprising administering to the cell or expressing in the cell a double-stranded inhibitory RNA (siRNA or miRNA), wherein the RNA comprises a subsequence of a sequence of the invention.

The invention provides isolated, synthetic or recombinant polypeptides comprising an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary polypeptide or peptide of the invention over a region of at least about 10, 15, 20, 25, 30, 35, 40, 45, 50, 55, 60, 65, 70, 75, 80, 85, 90, 95, 100, 125, 150, 175, 200, 225, 250, 275, 300, 325, 350 or more residues, or over the full length of the polypeptide. In one aspect, the sequence identities are determined by analysis with a sequence comparison algorithm or by a visual inspection. Exemplary polypeptide or peptide sequences of the invention include SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:143, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:209, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, SEQ ID NO:260, SEQ ID NO:262, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, SEQ ID NO:360, SEQ ID NO:362, SEQ ID NO:364, SEQ ID NO:366, SEQ ID NO:368, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419, SEQ ID NO:422, SEQ ID NO:424, SEQ ID NO:426, SEQ ID NO:428, SEQ ID NO:430, SEQ ID NO:432, SEQ ID NO:434, SEQ ID NO: 436, SEQ ID NO:438, SEQ ID NO:440, SEQ ID NO:442, SEQ ID NO:444, SEQ ID NO:446, SEQ ID NO:448, SEQ ID NO:450, SEQ ID NO:452, SEQ ID NO:454, SEQ ID NO:456, SEQ ID NO:458, SEQ ID NO:460, SEQ ID NO:462, SEQ ID NO:464, SEQ ID NO:466, SEQ ID NO:468, SEQ ID NO:470 and/or SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, including Tables 1 to 4, and all the sequences set forth in the Sequence Listing (all of these sequences are “exemplary enzymes/polypeptides of the invention”), and subsequences (including “enzymatically active fragments”) thereof (e.g., “peptides of the invention”) and variants thereof (all of these sequences encompassing polypeptide and peptide sequences of the invention).

Exemplary polypeptides also include fragments of at least about 10, 15, 20, 25, 30, 35, 40, 45, 50, 75, 80, 85, 90, 95, 100, 150, 200, 250, 300, 350, 400, 450, 500, 550, 600 or more residues in length, or over the full length of an enzyme. Polypeptide or peptide sequences of the invention include sequence encoded by a nucleic acid of the invention. Polypeptide or peptide sequences of the invention include polypeptides or peptides specifically bound by an antibody of the invention (e.g., epitopes), or polypeptides or peptides that can generate an antibody of the invention (e.g., an immunogen).

In one aspect, a polypeptide of the invention has at least one lignocellulosic enzyme, e.g., a glycosyl hydrolase, cellulase, endoglucanase, cellobiohydrolase, β-glucosidase(beta-glucosidase), xylanase, xylosidase (e.g., β-xylosidase) and/or arabinofuranosidase enzyme. In alternative aspects, a polynucleotide of the invention encodes a polypeptide that has at least one lignocellulosic enzyme activity activity.

In one aspect, the lignocellulosic enzyme activity, e.g., glycosyl hydrolase, cellulase, endoglucanase, cellobiohydrolase, mannanase, β-glucosidase(beta-glucosidase), xylanase, xylosidase (e.g., β-xylosidase) and/or arabinofuranosidase activity is thermostable. The polypeptide can retain a lignocellulosic enzyme activity under conditions comprising a temperature range about −100° C. to about −80° C., about −80° C. to about −40° C., about −40° C. to about −20° C., about −20° C. to about 0° C., about 0° C. to about 5° C., about 5° C. to about 15° C., about 15° C. to about 25° C., about 25° C. to about 37° C., about 37° C. to about 45° C., about 45° C. to about 55° C., about 55° C. to about 70° C., about 70° C. to about 75° C., about 75° C. to about 85° C., about 85° C. to about 90° C., about 90° C. to about 95° C., about 95° C. to about 100° C., about 100° C. to about 105° C., about 105° C. to about 110° C., about 110° C. to about 120° C., or 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C. or more. In some embodiments, the thermostable polypeptides according to the invention retain a lignocellulosic enzyme activity, at a temperature in the ranges described above, at about pH 3.0, about pH 3.5, about pH 4.0, about pH 4.5, about pH 5.0, about pH 5.5, about pH 6.0, about pH 6.5, about pH 7.0, about pH 7.5, about pH 8.0, about pH 8.5, about pH 9.0, about pH 9.5, about pH 10.0, about pH 10.5, about pH 11.0, about pH 11.5, about pH 12.0 or more.

In another aspect, the lignocellulosic enzyme activity can be thermotolerant. The polypeptide can retain a lignocellulosic enzyme activity after exposure to a temperature in the range from about −100° C. to about −80° C., about −80° C. to about −40° C., about −40° C. to about −20° C., about −20° C. to about 0° C., about 0° C. to about 5° C., about 5° C. to about 15° C., about 15° C. to about 25° C., about 25° C. to about 37° C., about 37° C. to about 45° C., about 45° C. to about 55° C., about 55° C. to about 70° C., about 70° C. to about 75° C., about 75° C. to about 85° C., about 85° C. to about 90° C., about 90° C. to about 95° C., about 95° C. to about 100° C., about 100° C. to about 105° C., about 105° C. to about 110° C., about 110° C. to about 120° C., or 95° C., 96° C., 97° C., 98° C., 99° C., 100° C., 101 ° C., 102° C., 103° C., 104° C., 105° C., 106° C., 107° C., 108° C., 109° C., 110° C., 111° C., 112° C., 113° C., 114° C., 115° C. or more. In some embodiments, the thermotolerant polypeptides according to the invention retain a lignocellulosic enzyme activity, after exposure to a temperature in the ranges described above, at about pH 3.0, about pH 3.5, about pH 4.0, about pH 4.5, about pH 5.0, about pH 5.5, about pH 6.0, about pH 6.5, about pH 7.0, about pH 7.5, about pH 8.0, about pH 8.5, about pH 9.0, about pH 9.5, about pH 10.0, about pH 10.5, about pH 11.0, about pH 11.5, about pH 12.0 or more.

Another aspect of the invention provides an isolated, synthetic or recombinant polypeptide or peptide comprising at least 10, 15, 20, 25, 30, 35, 40, 45, 50, 55, 60, 65, 70, 75, 80, 85, 90, 95, 100, 125, 150 or more consecutive bases of a polypeptide or peptide sequence of the invention, sequences substantially identical thereto, and the sequences complementary thereto. The peptide can be, e.g., an immunogenic fragment, a motif (e.g., a binding site), a signal sequence, a prepro sequence or an active site.

The invention provides isolated, synthetic or recombinant nucleic acids comprising a sequence encoding a polypeptide having a lignocellulosic activity, e.g., a glycosyl hydrolase, cellulase, endoglucanase, β-glucosidase(beta-glucosidase), mannanase, xylanase, xylosidase (e.g., β-xylosidase) and/or arabinofuranosidase enzyme activity and a signal sequence, wherein the nucleic acid comprises a sequence of the invention. The signal sequence can be derived from another the lignocellulosic enzyme, and/or glucose oxidase enzyme or a non-cellulase, e.g., non-endoglucanase, non-cellobiohydrolase, non-β-glucosidase (non-beta-glucosidase), non-xylanase, non-mannanase, non-β-xylosidase, non-arabinofuranosidase, and/or non-glucose oxidase (i.e., a heterologous) enzyme. The invention provides isolated, synthetic or recombinant nucleic acids comprising a sequence encoding a polypeptide having a lignocellulosic activity, and/or glucose oxidase enzyme activity, wherein the sequence does not contain a signal sequence and the nucleic acid comprises a sequence of the invention. In one aspect, the invention provides an isolated, synthetic or recombinant polypeptide comprising a polypeptide of the invention lacking all or part of a signal sequence. In one aspect, the isolated, synthetic or recombinant polypeptide can comprise the polypeptide of the invention comprising a heterologous signal sequence, such as a heterologous the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase; and/or glucose oxidase, enzyme signal sequence or non-cellulase, e.g., non-endoglucanase, non-cellobiohydrolase, non-β-glucosidase (non-beta-glucosidase), non-xylanase, non-mannanse, non-β-xylosidase, non-arabinofuranosidase signal sequence.

In one aspect, the invention provides chimeric (e.g., multidomain recombinant) proteins comprising a first domain comprising a signal sequence and/or a carbohydrate binding domain (CBM) of the invention and at least a second domain. The protein can be a fusion protein. The second domain can comprise an enzyme. The protein can be a non-enzyme, e.g., the chimeric protein can comprise a signal sequence and/or a CBM of the invention and a structural protein.

The invention provides chimeric polypeptides comprising (i) at least a first domain comprising (or consisting of) a carbohydrate binding domain (CBM), a signal peptide (SP), a prepro sequence and/or a catalytic domain (CD) of the invention; and, (ii) at least a second domain comprising a heterologous polypeptide or peptide, wherein the heterologous polypeptide or peptide is not naturally associated with the CBM, signal peptide (SP), prepro sequence and/ or catalytic domain (CD). In one aspect, the heterologous polypeptide or peptide is not a lignocellulosic enzyme, e.g., a glycosyl hydrolase, cellulase, endoglucanase, cellobiohydrolase, β-glucosidase(beta-glucosidase), xylanase, mannanse, xylosidase (e.g., β-xylosidase) and/or arabinofuranosidase enzyme. The heterologous polypeptide or peptide can be amino terminal to, carboxy terminal to or on both ends of the CBM, signal peptide (SP), prepro sequence and/or catalytic domain (CD).

The invention provides isolated, synthetic or recombinant nucleic acids encoding a chimeric polypeptide, wherein the chimeric polypeptide comprises at least a first domain comprising, or consisting of, a CBM, a signal peptide (SP), a prepro domain and/or a catalytic domain (CD) of the invention; and, at least a second domain comprising a heterologous polypeptide or peptide, wherein the heterologous polypeptide or peptide is not naturally associated with the CBM, signal peptide (SP), prepro domain and/ or catalytic domain (CD).

The invention provides isolated, synthetic or recombinant signal sequences (e.g., signal peptides) consisting of or comprising the sequence of (a sequence as set forth in) residues 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, 1 to 40, 1 to 41, 1 to 42, 1 to 43, 1 to 44, 1 to 45, 1 to 46 or 1 to 47, of a polypeptide of the invention, e.g., the exemplary polypeptides of the invention, e.g., SEQ ID NO:2, SEQ ID NO:4, etc., to SEQ ID NO:472 SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, (see Tables 1 to 4, and the sequence listing). In one aspect, the invention provides signal sequences comprising the first 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino terminal residues of a polypeptide of the invention.

In one aspect, the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme activity comprises a specific activity at about 37° C. in the range from about 1 to about 1200 units per milligram of protein, or, about 100 to about 1000 units per milligram of protein. In another aspect, the lignocellulosic enzyme activity comprises a specific activity from about 100 to about 1000 units per milligram of protein, or, from about 500 to about 750 units per milligram of protein. Alternatively, the lignocellulosic enzyme activity comprises a specific activity at 37° C. in the range from about 1 to about 750 units per milligram of protein, or, from about 500 to about 1200 units per milligram of protein. In one aspect, the lignocellulosic enzyme activity comprises a specific activity at 37° C. in the range from about 1 to about 500 units per milligram of protein, or, from about 750 to about 1000 units per milligram of protein. In another aspect, the lignocellulosic enzyme activity comprises a specific activity at 37° C. in the range from about 1 to about 250 units per milligram of protein. Alternatively, the lignocellulosic enzyme activity comprises a specific activity at 37° C. in the range from about 1 to about 100 units per milligram of protein.

In another aspect, the thermotolerance comprises retention of at least half of the specific activity of the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme at 37° C. after being heated to the elevated temperature. Alternatively, the thermotolerance can comprise retention of specific activity at 37° C. in the range from about 1 to about 1200 units per milligram of protein, or, from about 500 to about 1000 units per milligram of protein, after being heated to the elevated temperature. In another aspect, the thermotolerance can comprise retention of specific activity at 37° C. in the range from about 1 to about 500 units per milligram of protein after being heated to the elevated temperature.

The invention provides the isolated, synthetic or recombinant polypeptide of the invention, wherein the polypeptide comprises at least one glycosylation site. In one aspect, glycosylation can be an N-linked glycosylation. In one aspect, the polypeptide can be glycosylated after being expressed in a P. pastoris or a S. pombe.

In one aspect, the polypeptide can retain the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase activity under conditions comprising about pH 6.5, pH 6, pH 5.5, pH 5, pH 4.5 or pH 4 or more acidic. In another aspect, the polypeptide can retain the lignocellulosic enzyme activity under conditions comprising about pH 7, pH 7.5 pH 8.0, pH 8.5, pH 9, pH 9.5, pH 10, pH 10.5 or pH 11 or more basic pH. In one aspect, the polypeptide can retain the lignocellulosic enzyme activity after exposure to conditions comprising about pH 6.5, pH 6, pH 5.5, pH 5, pH 4.5 or pH 4 or more acidic pH. In another aspect, the polypeptide can retain the lignocellulosic enzyme activity after exposure to conditions comprising about pH 7, pH 7.5 pH 8.0, pH 8.5, pH 9, pH 9.5, pH 10, pH 10.5 or pH 11 or more basic pH.

In one aspect, the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme of the invention has activity at under alkaline conditions, e.g., the alkaline conditions of the gut, e.g., the small intestine. In one aspect, the polypeptide can retains activity after exposure to the acidic pH of the stomach.

The invention provides protein preparations comprising a polypeptide (including peptides) of the invention, wherein the protein preparation comprises a liquid, a solid or a gel. The invention provides heterodimers comprising a polypeptide of the invention and a second protein or domain. The second member of the heterodimer can be a different the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme, a different enzyme or another protein. In one aspect, the second domain can be a polypeptide and the heterodimer can be a fusion protein. In one aspect, the second domain can be an epitope or a tag. In one aspect, the invention provides homodimers comprising a polypeptide of the invention.

The invention provides immobilized polypeptides (including peptides) having the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme activity, wherein the immobilized polypeptide comprises a polypeptide of the invention, a polypeptide encoded by a nucleic acid of the invention, or a polypeptide comprising a polypeptide of the invention and a second domain. In one aspect, the polypeptide can be immobilized on a cell, a metal, a resin, a polymer, a ceramic, a glass, a microelectrode, a graphitic particle, a bead, a gel, a plate, an array or a capillary tube.

The invention also provides arrays comprising an immobilized nucleic acid of the invention, including, e.g., probes of the invention. The invention also provides arrays comprising an antibody of the invention.

The invention provides isolated, synthetic or recombinant antibodies that specifically bind to a polypeptide of the invention or to a polypeptide encoded by a nucleic acid of the invention.

These antibodies of the invention can be a monoclonal or a polyclonal antibody. The invention provides hybridomas comprising an antibody of the invention, e.g., an antibody that specifically binds to a polypeptide of the invention or to a polypeptide encoded by a nucleic acid of the invention. The invention provides nucleic acids encoding these antibodies.

The invention provides method of isolating or identifying a polypeptide having the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase activity comprising the steps of: (a) providing an antibody of the invention; (b) providing a sample comprising polypeptides; and (c) contacting the sample of step (b) with the antibody of step (a) under conditions wherein the antibody can specifically bind to the polypeptide, thereby isolating or identifying a polypeptide having the lignocellulosic enzyme activity.

The invention provides methods of making an anti-glucose oxidase, an anti-cellulase, e.g., anti-endoglucanase, anti-cellobiohydrolase, anti-β-glucosidase (anti-beta-glucosidase), anti-xylanase, anti-mannanse, anti-β-xylosidase or anti-arabinofuranosidase enzyme antibody comprising administering to a non-human animal a nucleic acid of the invention or a polypeptide of the invention or subsequences thereof in an amount sufficient to generate a humoral immune response, thereby making an anti-glucose oxidase or anti-cellulase, e.g., anti-endoglucanase, anti-cellobiohydrolase, anti-β-glucosidase (anti-beta-glucosidase), anti-xylanase, anti-mannanse, anti-β-xylosidase, and/or anti-arabinofuranosidase enzyme antibody. The invention provides methods of making an anti-glucose oxidase or anti-cellulase, e.g., anti-endoglucanase, anti-cellobiohydrolase, anti-β-glucosidase (anti-beta-glucosidase), anti-xylanase, anti-mannanse, anti-β-xylosidase, and/or anti-arabinofuranosidase immune response (cellular or humoral) comprising administering to a non-human animal a nucleic acid of the invention or a polypeptide of the invention or subsequences thereof in an amount sufficient to generate an immune response (cellular or humoral).

The invention provides methods of producing a recombinant polypeptide comprising the steps of: (a) providing a nucleic acid of the invention operably linked to a promoter; and (b) expressing the nucleic acid of step (a) under conditions that allow expression of the polypeptide, thereby producing a recombinant polypeptide. In one aspect, the method can further comprise transforming a host cell with the nucleic acid of step (a) followed by expressing the nucleic acid of step (a), thereby producing a recombinant polypeptide in a transformed cell.

The invention provides methods for identifying a polypeptide having the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme activity comprising the following steps: (a) providing a polypeptide of the invention; or a polypeptide encoded by a nucleic acid of the invention; (b) providing the lignocellulosic enzyme substrate; and (c) contacting the polypeptide or a fragment or variant thereof of step (a) with the substrate of step (b) and detecting a decrease in the amount of substrate or an increase in the amount of a reaction product, wherein a decrease in the amount of the substrate or an increase in the amount of the reaction product detects a polypeptide having the lignocellulosic enzyme activity. In one aspect, the substrate is a cellulose-comprising or a polysaccharide-comprising (e.g., soluble cellooligsaccharide- and/or arabinoxylan oligomer-comprising) compound.

The invention provides methods for identifying a lignocellulosic enzyme, e.g., a glycosyl hydrolase, cellulase, endoglucanase, cellobiohydrolase, β-glucosidase(beta-glucosidase), xylanase, mannanse, β-xylosidase and/or arabinofuranosidase enzyme substrate comprising the following steps: (a) providing a polypeptide of the invention; or a polypeptide encoded by a nucleic acid of the invention; (b) providing a test substrate; and (c) contacting the polypeptide of step (a) with the test substrate of step (b) and detecting a decrease in the amount of substrate or an increase in the amount of reaction product, wherein a decrease in the amount of the substrate or an increase in the amount of a reaction product identifies the test substrate as a lignocellulosic enzyme substrate.

The invention provides methods of determining whether a test compound specifically binds to a polypeptide comprising the following steps: (a) expressing a nucleic acid or a vector comprising the nucleic acid under conditions permissive for translation of the nucleic acid to a polypeptide, wherein the nucleic acid comprises a nucleic acid of the invention, or, providing a polypeptide of the invention; (b) providing a test compound; (c) contacting the polypeptide with the test compound; and (d) determining whether the test compound of step (b) specifically binds to the polypeptide.

The invention provides methods for identifying a modulator of a lignocellulosic enzyme, e.g., a glycosyl hydrolase, cellulase, endoglucanase, cellobiohydrolase, β-glucosidase(beta-glucosidase), xylanase, mannanse, β-xylosidase and/or arabinofuranosidase enzyme activity comprising the following steps: (a) providing a polypeptide of the invention or a polypeptide encoded by a nucleic acid of the invention; (b) providing a test compound; (c) contacting the polypeptide of step (a) with the test compound of step (b) and measuring an activity of the lignocellulosic enzyme, wherein a change in the lignocellulosic enzyme activity measured in the presence of the test compound compared to the activity in the absence of the test compound provides a determination that the test compound modulates the lignocellulosic enzyme activity. In one aspect, the lignocellulosic enzyme activity can be measured by providing a lignocellulosic enzyme substrate and detecting a decrease in the amount of the substrate or an increase in the amount of a reaction product, or, an increase in the amount of the substrate or a decrease in the amount of a reaction product. A decrease in the amount of the substrate or an increase in the amount of the reaction product with the test compound as compared to the amount of substrate or reaction product without the test compound identifies the test compound as an activator of the lignocellulosic enzyme activity. An increase in the amount of the substrate or a decrease in the amount of the reaction product with the test compound as compared to the amount of substrate or reaction product without the test compound identifies the test compound as an inhibitor of the lignocellulosic enzyme activity.

The invention provides computer systems comprising a processor and a data storage device wherein said data storage device has stored thereon a polypeptide sequence or a nucleic acid sequence of the invention (e.g., a polypeptide or peptide encoded by a nucleic acid of the invention). In one aspect, the computer system can further comprise a sequence comparison algorithm and a data storage device having at least one reference sequence stored thereon. In another aspect, the sequence comparison algorithm comprises a computer program that indicates polymorphisms. In one aspect, the computer system can further comprise an identifier that identifies one or more features in said sequence. The invention provides computer readable media having stored thereon a polypeptide sequence or a nucleic acid sequence of the invention. The invention provides methods for identifying a feature in a sequence comprising the steps of: (a) reading the sequence using a computer program which identifies one or more features in a sequence, wherein the sequence comprises a polypeptide sequence or a nucleic acid sequence of the invention; and (b) identifying one or more features in the sequence with the computer program. The invention provides methods for comparing a first sequence to a second sequence comprising the steps of: (a) reading the first sequence and the second sequence through use of a computer program which compares sequences, wherein the first sequence comprises a polypeptide sequence or a nucleic acid sequence of the invention; and (b) determining differences between the first sequence and the second sequence with the computer program. The step of determining differences between the first sequence and the second sequence can further comprise the step of identifying polymorphisms. In one aspect, the method can further comprise an identifier that identifies one or more features in a sequence. In another aspect, the method can comprise reading the first sequence using a computer program and identifying one or more features in the sequence.

The invention provides methods for isolating or recovering a nucleic acid encoding a polypeptide having the lignocellulosic enzyme, e.g., glycosyl hydrolase, cellulase, endoglucanase, cellobiohydrolase, β-glucosidase(beta-glucosidase), xylanase, mannanse, β-xylosidase and/or arabinofuranosidase enzyme activity from a sample, e.g. an environmental sample, comprising the steps of: (a) providing an amplification primer sequence pair for amplifying a nucleic acid encoding a polypeptide having a lignocellulosic activity, wherein the primer pair is capable of amplifying a nucleic acid of the invention; (b) isolating a nucleic acid from the sample, e.g. environmental sample, or treating the sample, e.g. environmental sample, such that nucleic acid in the sample is accessible for hybridization to the amplification primer pair; and, (c) combining the nucleic acid of step (b) with the amplification primer pair of step (a) and amplifying nucleic acid from the sample, e.g. environmental sample, thereby isolating or recovering a nucleic acid encoding a polypeptide having a lignocellulosic activity from a sample, e.g. an environmental sample. One or each member of the amplification primer sequence pair can comprise an oligonucleotide comprising an amplification primer sequence pair of the invention, e.g., having at least about 10 to 50 consecutive bases of a sequence of the invention.

The invention provides methods for isolating or recovering a nucleic acid encoding a polypeptide having a lignocellulosic activity, e.g., a glycosyl hydrolase, cellulase, endoglucanase, β-glucosidase(beta-glucosidase), xylanase, mannanse, β-xylosidase and/or arabinofuranosidase enzyme activity from a sample, e.g. an environmental sample, comprising the steps of: (a) providing a polynucleotide probe comprising a nucleic acid of the invention or a subsequence thereof; (b) isolating a nucleic acid from the sample, e.g. environmental sample, or treating the sample, e.g. environmental sample, such that nucleic acid in the sample is accessible for hybridization to a polynucleotide probe of step (a); (c) combining the isolated nucleic acid or the treated sample, e.g. environmental sample, of step (b) with the polynucleotide probe of step (a); and (d) isolating a nucleic acid that specifically hybridizes with the polynucleotide probe of step (a), thereby isolating or recovering a nucleic acid encoding a polypeptide having a lignocellulosic activity from a sample, e.g. an environmental sample. The sample, e.g. environmental sample, can comprise a water sample, a liquid sample, a soil sample, an air sample or a biological sample. In one aspect, the biological sample can be derived from a bacterial cell, a protozoan cell, an insect cell, a yeast cell, a plant cell, a fungal cell or a mammalian cell.

The invention provides methods of generating a variant of a nucleic acid encoding a polypeptide having a lignocellulosic activity, e.g., a glycosyl hydrolase, cellulase, endoglucanase, β-glucosidase(beta-glucosidase), xylanase, mannanse, β-xylosidase and/or arabinofuranosidase enzyme activity comprising the steps of: (a) providing a template nucleic acid comprising a nucleic acid of the invention; and (b) modifying, deleting or adding one or more nucleotides in the template sequence, or a combination thereof, to generate a variant of the template nucleic acid. In one to aspect, the method can further comprise expressing the variant nucleic acid to generate a variant the lignocellulosic enzyme polypeptide. The modifications, additions or deletions can be introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GENE SITE SATURATION MUTAGENESIS (or GSSM), synthetic ligation reassembly (SLR), Chromosomal Saturation Mutagenesis (CSM) or a combination thereof. In another aspect, the modifications, additions or deletions are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

In one aspect, the method can be iteratively repeated until a lignocellulosic enzyme, e.g., a glycosyl hydrolase, cellulase, endoglucanase, cellobiohydrolase, β-glucosidase(beta-glucosidase), xylanase, mannanse, β-xylosidase and/or arabinofuranosidase enzyme having an altered or different activity or an altered or different stability from that of a polypeptide encoded by the template nucleic acid is produced. In one aspect, the variant the lignocellulosic enzyme polypeptide is thermotolerant, and retains some activity after being exposed to an elevated temperature. In another aspect, the variant the lignocellulosic enzyme polypeptide has increased glycosylation as compared to the lignocellulosic enzyme encoded by a template nucleic acid. Alternatively, the variant the polypeptide has a lignocellulosic enzyme activity under a high temperature, wherein the lignocellulosic enzyme encoded by the template nucleic acid is not active under the high temperature. In one aspect, the method can be iteratively repeated until a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme coding sequence having an altered codon usage from that of the template nucleic acid is produced. In another aspect, the method can be iteratively repeated until a lignocellulosic enzyme gene having higher or lower level of message expression or stability from that of the template nucleic acid is produced.

The invention provides methods for modifying codons in a nucleic acid encoding a polypeptide having a lignocellulosic activity, e.g., a glycosyl hydrolase, cellulase, endoglucanase, beta-glucosidase, xylanase, mannanse, β-xylosidase and/or arabinofuranosidase enzyme activity to increase its expression in a host cell, the method comprising the following steps: (a) providing a nucleic acid of the invention encoding a polypeptide having a lignocellulosic enzyme activity; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying codons in a nucleic acid encoding a polypeptide having a lignocellulosic activity, e.g., a glycosyl hydrolase, cellulase, endoglucanase, beta-glucosidase, xylanase, mannanse, β-xylosidase and/or arabinofuranosidase enzyme activity; the method comprising the following steps: (a) providing a nucleic acid of the invention; and, (b) identifying a codon in the nucleic acid of step (a) and replacing it with a different codon encoding the same amino acid as the replaced codon, thereby modifying codons in a nucleic acid encoding a lignocellulosic enzyme.

The invention provides methods for modifying codons in a nucleic acid encoding a polypeptide having a lignocellulosic activity, e.g., a glycosyl hydrolase, cellulase, endoglucanase, beta-glucosidase, xylanase, mannanse, β-xylosidase and/or arabinofuranosidase enzyme activity to increase its expression in a host cell, the method comprising the following steps: (a) providing a nucleic acid of the invention encoding a lignocellulosic enzyme polypeptide; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying a codon in a nucleic acid encoding a polypeptide having a lignocellulosic activity, e.g., a glycosyl hydrolase, cellulase, endoglucanase, beta-glucosidase, xylanase, mannanse, β-xylosidase and/or arabinofuranosidase enzyme activity to decrease its expression in a host cell, the method comprising the following steps: (a) providing a nucleic acid of the invention; and (b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell. In one aspect, the host cell can be a bacterial cell, a fungal cell, an insect cell, a yeast cell, a plant cell or a mammalian cell.

The invention provides methods for producing a library of nucleic acids encoding a plurality of modified the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme active sites or substrate binding sites, wherein the modified active sites or substrate binding sites are derived from a first nucleic acid comprising a sequence encoding a first active site or a first substrate binding site the method comprising the following steps: (a) providing a first nucleic acid encoding a first active site or first substrate binding site, wherein the first nucleic acid sequence comprises a sequence that hybridizes under stringent conditions to a nucleic acid of the invention, and the nucleic acid encodes a lignocellulosic enzyme active site or a lignocellulosic enzyme substrate binding site; (b) providing a set of mutagenic oligonucleotides that encode naturally-occurring amino acid variants at a plurality of targeted codons in the first nucleic acid; and, (c) using the set of mutagenic oligonucleotides to generate a set of active site-encoding or substrate binding site-encoding variant nucleic acids encoding a range of amino acid variations at each amino acid codon that was mutagenized, thereby producing a library of nucleic acids encoding a plurality of modified the lignocellulosic enzyme active sites or substrate binding sites. In one aspect, the method comprises mutagenizing the first nucleic acid of step (a) by a method comprising an optimized directed evolution system, GENE SITE SATURATION MUTAGENESIS™ (or GSSM), synthetic ligation reassembly (SLR), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, and a combination thereof. In another aspect, the method comprises mutagenizing the first nucleic acid of step (a) or variants by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

The invention provides methods for making a small molecule comprising the following steps: (a) providing a plurality of biosynthetic enzymes capable of synthesizing or modifying a small molecule, wherein one of the enzymes comprises a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme encoded by a nucleic acid of the invention; (b) providing a substrate for at least one of the enzymes of step (a); and (c) reacting the substrate of step (b) with the enzymes under conditions that facilitate a plurality of biocatalytic reactions to generate a small molecule by a series of biocatalytic reactions. The invention provides methods for modifying a small molecule comprising the following steps: (a) providing a lignocellulosic enzyme, wherein the enzyme comprises a polypeptide of the invention, or, a polypeptide encoded by a nucleic acid of the invention, or a subsequence thereof; (b) providing a small molecule; and (c) reacting the enzyme of step (a) with the small molecule of step (b) under conditions that facilitate an enzymatic reaction catalyzed by the lignocellulosic enzyme, thereby modifying a small molecule by a lignocellulosic enzymatic reaction. In one aspect, the method can comprise a plurality of small molecule substrates for the enzyme of step (a), thereby generating a library of modified small molecules produced by at least one enzymatic reaction catalyzed by the lignocellulosic enzyme. In one aspect, the method can comprise a plurality of additional enzymes under conditions that facilitate a plurality of biocatalytic reactions by the enzymes to form a library of modified small molecules produced by the plurality of enzymatic reactions. In another aspect, the method can further comprise the step of testing the library to determine if a particular modified small molecule that exhibits a desired activity is present within the library. The step of testing the library can further comprise the steps of systematically eliminating all but one of the biocatalytic reactions used to produce a portion of the plurality of the modified small molecules within the library by testing the portion of the modified small molecule for the presence or absence of the particular modified small molecule with a desired activity, and identifying at least one specific biocatalytic reaction that produces the particular modified small molecule of desired activity.

The invention provides methods for determining a functional fragment of an enzyme of the invention comprising the steps of: (a) providing a polypeptide of the invention, or a polypeptide encoded by a nucleic acid of the invention, or a subsequence thereof; and (b) deleting a plurality of amino acid residues from the sequence of step (a) and testing the remaining subsequence for lignocellulosic enzyme activity, thereby determining a functional fragment of the enzyme. In one aspect, lignocellulosic enzyme activity, is measured by providing a substrate and detecting a decrease in the amount of the substrate or an increase in the amount of a reaction product.

The invention provides methods for whole cell engineering of new or modified phenotypes by using real-time metabolic flux analysis, the method comprising the following steps: (a) making a modified cell by modifying the genetic composition of a cell, wherein the genetic composition is modified by addition to the cell of a nucleic acid of the invention; (b) culturing the modified cell to generate a plurality of modified cells; (c) measuring at least one metabolic parameter of the cell by monitoring the cell culture of step (b) in real time; and, (d) analyzing the data of step (c) to determine if the measured parameter differs from a comparable measurement in an unmodified cell under similar conditions, thereby identifying an engineered phenotype in the cell using real-time metabolic flux analysis. In one aspect, the genetic composition of the cell can be modified by a method comprising deletion of a sequence or modification of a sequence in the cell, or, knocking out the expression of a gene. In one aspect, the method can further comprise selecting a cell comprising a newly engineered phenotype. In another aspect, the method can comprise culturing the selected cell, thereby generating a new cell strain comprising a newly engineered phenotype.

The invention provides methods of increasing thermotolerance or thermostability of a lignocellulosic enzyme, the method comprising glycosylating a lignocellulosic enzyme polypeptide, wherein the polypeptide comprises at least thirty contiguous amino acids of a polypeptide of the invention; or a polypeptide encoded by a nucleic acid sequence of the invention, thereby increasing the thermotolerance or thermostability of the lignocellulosic enzyme polypeptide. In one aspect, the lignocellulosic enzyme specific activity can be thermostable or thermotolerant at a temperature in the range from greater than about 37° C. to about 95° C.

The invention provides methods for overexpressing a recombinant glucose oxidase and/or the lignocellulosic enzyme polypeptide in a cell comprising expressing a vector comprising a nucleic acid comprising a nucleic acid of the invention or a nucleic acid sequence of the invention, wherein the sequence identities are determined by analysis with a sequence comparison algorithm or by visual inspection, wherein overexpression is effected by use of a high activity promoter, a dicistronic vector or by gene amplification of the vector.

The invention provides methods of making a transgenic plant comprising the following steps: (a) introducing a heterologous nucleic acid sequence into the cell, wherein the heterologous nucleic sequence comprises a nucleic acid sequence of the invention, thereby producing a transformed plant cell; and (b) producing a transgenic plant from the transformed cell. In one aspect, the step (a) can further comprise introducing the heterologous nucleic acid sequence by electroporation or microinjection of plant cell protoplasts. In another aspect, the step (a) can further comprise introducing the heterologous nucleic acid sequence directly to plant tissue by DNA particle bombardment. Alternatively, the step (a) can further comprise introducing the heterologous nucleic acid sequence into the plant cell DNA using an Agrobacterium tumefaciens host. In one aspect, the plant cell can be a cane sugar, beet, soybean, tomato, potato, corn, rice, wheat, tobacco or barley cell. The cell can be derived from a monocot or a dicot, or a monocot corn, sugarcane, rice, wheat, barley, switchgrass or Miscanthus; or a dicot oilseed crop, soy, canola, rapeseed, flax, cotton, palm oil, sugar beet, peanut, tree, poplar or lupine.

The invention provides methods of expressing a heterologous nucleic acid sequence in a plant cell comprising the following steps: (a) transforming the plant cell with a heterologous nucleic acid sequence operably linked to a promoter, wherein the heterologous nucleic sequence comprises a nucleic acid of the invention; (b) growing the plant under conditions wherein the heterologous nucleic acids sequence is expressed in the plant cell. The invention provides methods of expressing a heterologous nucleic acid sequence in a plant cell comprising the following steps: (a) transforming the plant cell with a heterologous nucleic acid sequence operably linked to a promoter, wherein the heterologous nucleic sequence comprises a sequence of the invention; (b) growing the plant under conditions wherein the heterologous nucleic acids sequence is expressed in the plant cell. In one aspect, the promoter is or comprises: a viral, bacterial, mammalian or plant promoter; or, a plant promoter; or, a potato, rice, corn, wheat, tobacco or barley promoter; or, a constitutive promoter or a CaMV35S promoter; or, an inducible promoter; or, a tissue-specific promoter or an environmentally regulated or a developmentally regulated promoter; or, a seed-specific, a leaf-specific, a root-specific, a stem-specific or an abscission-induced promoter; or, a seed preferred promoter, a maize gamma zein promoter or a maize ADP-gpp promoter. In one aspect, the plant cell is derived from is a monocot or dicot, or the plant is a monocot corn, sugarcane, rice, wheat, barley, switchgrass or Miscanthus; or the plant is a dicot oilseed crop, soy, canola, rapeseed, flax, cotton, palm oil, sugar beet, peanut, tree, poplar or lupine.

The invention provides methods for hydrolyzing, breaking up or disrupting a cellooligsaccharide, an arabinoxylan oligomer, or a glucan- or cellulose-comprising composition comprising the following steps: (a) providing a polypeptide of the invention; (b) providing a composition comprising a cellulose or a glucan; and (c) contacting the polypeptide of step (a) with the composition of step (b) under conditions wherein the cellulase hydrolyzes, breaks up or disrupts the cellooligsaccharide, arabinoxylan oligomer, or glucan- or cellulose-comprising composition; wherein optionally the composition comprises a plant cell, a bacterial cell, a yeast cell, an insect cell, or an animal cell. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides feeds or foods comprising a polypeptide of the invention, or a polypeptide encoded by a nucleic acid of the invention. In one aspect, the invention provides a food, feed, a liquid, e.g., a beverage (such as a fruit juice or a beer), a bread or a dough or a bread product, or a beverage precursor (e.g., a wort), comprising a polypeptide of the invention. The invention provides food or nutritional supplements for an animal comprising a polypeptide of the invention, e.g., a polypeptide encoded by the nucleic acid of the invention. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

In one aspect, the polypeptide in the food or nutritional supplement can be glycosylated. The invention provides edible enzyme delivery matrices comprising a polypeptide of the invention, e.g., a polypeptide encoded by the nucleic acid of the invention. In one aspect, the delivery matrix comprises a pellet. In one aspect, the polypeptide can be glycosylated. In one aspect, the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme activity is thermotolerant. In another aspect, the lignocellulosic enzyme activity is thermostable.

The invention provides a food, a feed or a nutritional supplement comprising a polypeptide of the invention. The invention provides methods for utilizing a lignocellulosic enzyme of the invention, e.g., a glycosyl hydrolase, cellulase, endoglucanase, cellobiohydrolase, beta-glucosidase, xylanase, mannanse, β-xylosidase and/or arabinofuranosidase enzyme, as a nutritional supplement in an animal or human diet, the method comprising: preparing a nutritional supplement containing a lignocellulosic enzyme of the invention comprising at least thirty contiguous amino acids of a polypeptide of the invention; and administering the nutritional supplement to an animal. The animal can be a human, a ruminant or a monogastric animal. The lignocellulosic enzyme can be prepared by expression of a polynucleotide encoding the lignocellulosic enzyme in a host organism, e.g., a bacterium, a yeast, a plant, an insect, a fungus and/or an animal. The organism also can be an S. pombe, S. cerevisiae, Pichia pastoris, E. coli, Streptomyces sp., Bacillus sp. and/or Lactobacillus sp. In one aspect, the plant is a monocot or dicot, or the plant is a monocot corn, sugarcane, rice, wheat, barley, switchgrass or Miscanthus; or the plant is a dicot oilseed crop, soy, canola, rapeseed, flax, cotton, palm oil, sugar beet, peanut, tree, poplar or lupine.

The invention provides edible enzyme delivery matrix comprising a thermostable recombinant of a lignocellulosic enzyme of the invention, e.g., a glycosyl hydrolase, cellulase, endoglucanase, cellobiohydrolase, beta-glucosidase, xylanase, mannanse, β-xylosidase and/or arabinofuranosidase enzyme of the invention. The invention provides methods for delivering a lignocellulosic enzyme supplement to an animal or human, the method comprising: preparing an edible enzyme delivery matrix in the form of pellets comprising a granulate edible carrier and a thermostable recombinant the lignocellulosic enzyme, wherein the pellets readily disperse the lignocellulosic enzyme contained therein into aqueous media, and administering the edible enzyme delivery matrix to the animal. The recombinant lignocellulosic enzyme of the invention can comprise all or a subsequence of at least one polypeptide of the invention. The lignocellulosic enzyme can be glycosylated to provide thermostability at pelletizing conditions. The delivery matrix can be formed by pelletizing a mixture comprising a grain germ and a lignocellulosic enzyme. The pelletizing conditions can include application of steam. The pelletizing conditions can comprise application of a temperature in excess of about 80° C. for about 5 minutes and the enzyme retains a specific activity of at least 350 to about 900 units per milligram of enzyme.

In one aspect, invention provides a pharmaceutical composition comprising a lignocellulosic enzyme of the invention, e.g., a glycosyl hydrolase, cellulase, endoglucanase, cellobiohydrolase, beta-glucosidase, xylanase, mannanse, β-xylosidase and/or arabinofuranosidase enzyme of the invention, or a polypeptide encoded by a nucleic acid of the invention. In one aspect, the pharmaceutical composition acts as a digestive aid.

In certain aspects, a cellulose-containing compound is contacted a polypeptide of the invention having a lignocellulosic enzyme of the invention at a pH in the range of between about pH 3.0 to 9.0, 10.0, 11.0 or more. In other aspects, a cellulose-containing compound is contacted with the lignocellulosic enzyme at a temperature of about 55° C., 60° C., 65° C., 70° C., 75° C., 80° C., 85° C., 90° C., or more.

The invention provides methods for delivering an enzyme supplement, e.g., a glycosyl hydrolase, cellulase, endoglucanase, cellobiohydrolase, beta-glucosidase, xylanase, mannanse, β-xylosidase and/or arabinofuranosidase supplement; and/or glucose oxidase supplement, to an animal or human, the method comprising: preparing an edible enzyme delivery matrix or pellets comprising a granulate edible carrier and a thermostable recombinant enzyme of the invention, wherein the pellets readily disperse the cellulase enzyme contained therein into aqueous media, and the recombinant enzyme of the invention, or a polypeptide encoded by a nucleic acid of the invention; and, administering the edible enzyme delivery matrix or pellet to the animal; and optionally the granulate edible carrier comprises a carrier selected from the group consisting of a grain germ, a grain germ that is spent of oil, a hay, an alfalfa, a timothy, a soy hull, a sunflower seed meal and a wheat midd, and optionally the edible carrier comprises grain germ that is spent of oil, and optionally the enzyme of the invention is glycosylated to provide thermostability at pelletizing conditions, and optionally the delivery matrix is formed by pelletizing a mixture comprising a grain germ and a cellulase, and optionally the pelletizing conditions include application of steam, and optionally the pelletizing conditions comprise application of a temperature in excess of about 80° C. for about 5 minutes and the enzyme retains a specific activity of at least 350 to about 900 units per milligram of enzyme.

The invention provides cellulose- or cellulose derivative-compositions comprising a polypeptide of the invention, or a polypeptide encoded by a nucleic acid of the invention, wherein in alternative embodiments the polypeptide has a glycosyl hydrolase, glucose oxidase, cellulase, endoglucanase, cellobiohydrolase, beta-glucosidase, xylanase, mannanse, β-xylosidase, and/or an arabinofuranosidase activity.

The invention provides wood, wood pulp or wood products, or wood waste, comprising an enzyme of the invention, or an enzyme encoded by a nucleic acid of the invention, wherein optionally the activity of the enzyme of the invention comprises endoglucanase, cellobiohydrolase, beta-glucosidase, xylanase, mannanse, β-xylosidase and/or arabinofuranosidase activity.

The invention provides paper, paper pulp or paper products, or paper waste byproducts or recycled material, comprising a polypeptide of the invention, or a polypeptide encoded by a nucleic acid of the invention, wherein optionally the polypeptide has glycosyl hydrolase, cellulase, endoglucanase, cellobiohydrolase, beta-glucosidase, xylanase, mannanse, β-xylosidase and/or arabinofuranosidase activity.

The invention provides methods for reducing the amount of cellulose in a paper, a wood or wood product comprising contacting the paper, wood or wood product, or wood waste, with an enzyme of the invention, or an enzyme encoded by a nucleic acid of the invention, wherein optionally the enzyme activity comprises a glycosyl hydrolase, endoglucanase, cellobiohydrolase, beta-glucosidase, xylanase, mannanse, β-xylosidase and/or arabinofuranosidase activity.

The invention provides detergent compositions comprising an enzyme of the invention, or an enzyme encoded by a nucleic acid of the invention, wherein optionally the polypeptide is formulated in a non-aqueous liquid composition, a cast solid, a granular form, a particulate form, a compressed tablet, a gel form, a paste or a slurry form. In one aspect, the activity comprises a glycosyl hydrolase, endoglucanase, cellobiohydrolase, beta-glucosidase, xylanase, mannanse, β-xylosidase and/or arabinofuranosidase activity.

The invention provides pharmaceutical compositions or dietary supplements comprising an enzyme of the invention, or a cellulase encoded by a nucleic acid of the invention, wherein optionally the enzyme is formulated as a tablet, gel, pill, implant, liquid, spray, powder, food, feed pellet or as an encapsulated formulation. In one aspect, the activity comprises a glycosyl hydrolase, endoglucanase, cellobiohydrolase, beta-glucosidase, xylanase, mannanse, β-xylosidase and/or arabinofuranosidase activity.

The invention provides fuels comprising a polypeptide of the invention, or a polypeptide encoded by a nucleic acid of the invention, wherein optionally the fuel is derived from a plant material, which optionally comprises potatoes, soybean (rapeseed), barley, rye, corn, oats, wheat, beets or sugar cane. The plant material can be derived from a monocot or a dicot, or a monocot corn, sugarcane, rice, wheat, barley, switchgrass or Miscanthus; or a dicot oilseed crop, soy, canola, rapeseed, flax, cotton, palm oil, sugar beet, peanut, tree, poplar or lupine. The fuel can comprise a bioalcohol, e.g., a bioethanol or a gasoline-ethanol mix, a biomethanol or a gasoline-methanol mix, a biobutanol or a gasoline-butanol mix, or a biopropanol or a gasoline-propanol mix. In one aspect, the activity comprises a glycosyl hydrolase, endoglucanase, cellobiohydrolase, beta-glucosidase, xylanase, mannanse, β-xylosidase and/or arabinofuranosidase activity.

The invention provides methods for making a fuel or alcohol comprising contacting an enzyme of the invention, or a composition comprising an enzyme of the invention, or a polypeptide encoded by a nucleic acid of the invention, or any one of the mixtures or “cocktails” or products of manufacture of the invention, with a biomass, e.g., a composition comprising a cellulose, a fermentable sugar or polysaccharide, such as a lignocellulosic material. In alternative embodiments, the composition comprising cellulose or a fermentable sugar comprises a plant, plant product, plant waste or plant derivative, and the plant, plant waste or plant product can comprise cane sugar plants or plant products, beets or sugarbeets, wheat, corn, soybeans, potato, rice or barley. In alternative embodiments, the fuel comprises a bioethanol or a gasoline-ethanol mix, a biomethanol or a gasoline-methanol mix, a biobutanol or a gasoline-butanol mix, or a biopropanol or a gasoline-propanol mix. The enzyme of the invention of the invention can be part of a plant or seed, e.g., a transgenic plant or seed—and in one aspect, the enzyme of the invention is expressed as a heterologous recombinant enzyme in the very biomass (e.g., plant, seed, plant waste) which is targeted for hydrolysis and conversion into a fuel or alcohol by this method of the invention. In one aspect, the activity comprises a glycosyl hydrolase, endoglucanase, cellobiohydrolase, beta-glucosidase, xylanase, mannanse, β-xylosidase and/or arabinofuranosidase activity.

The invention provides methods for making biofuel, e.g., comprising or consisting of a bioalcohol such as bioethanol, biomethanol, biobutanol or biopropanol, or a mixture thereof, comprising contacting a composition comprising an enzyme of the invention, or a fermentable sugar or lignocellulosic material comprising a polypeptide of the invention, or a polypeptide encoded by a nucleic acid of the invention, or any one of the mixtures or “cocktails” or products of manufacture of the invention, with a biomass, e.g., a composition comprising a cellulose, a fermentable sugar or polysaccharide, such as a lignocellulosic material. In alternative embodiments, the composition comprising the enzyme of the invention, and/or the material to be hydrolyzed, comprises a plant, plant waste, plant product or plant derivative. In alternative embodiments, the plant, plant waste or plant product comprises cane sugar plants or plant products (e.g., cane tops), beets or sugarbeets, wheat, corn, soybeans, potato, rice or barley. In one aspect, the plant is a monocot or dicot, or the plant is a monocot corn, sugarcane (including a cane part, e.g., cane tops), rice, wheat, barley, switchgrass or Miscanthus; or the plant is a dicot oilseed crop, soy, canola, rapeseed, flax, cotton, palm oil, sugar beet, peanut, tree, poplar or lupine. In one aspect, enzyme of the invention has an activity comprising a glycosyl hydrolase, endoglucanase, cellobiohydrolase, beta-glucosidase, xylanase, mannanse, β-xylosidase and/or arabinofuranosidase activity.

The invention provides enzyme ensembles, or “cocktail”, for depolymerization of cellulosic and hemicellulosic polymers to metabolizeable carbon moieties comprising a polypeptide of the invention, or a polypeptide encoded by a nucleic acid of the invention. In one aspect, enzyme of the invention has an activity comprising a glycosyl hydrolase, endoglucanase, cellobiohydrolase, beta-glucosidase, xylanase, mannanse, β-xylosidase and/or arabinofuranosidase activity. The enzyme ensembles, or “cocktails”, of the invention can be in the form of a composition (e.g., a formulation, liquid or solid), e.g., as a product of manufacture.

The invention provides compositions (including products of manufacture, enzyme ensembles, or “cocktails”) comprising (a) a mixture (or “cocktail”, “an enzyme ensemble”, a product of manufacture) of lignocellulosic enzymes, e.g., hemicellulose- and cellulose-hydrolyzing enzymes, including at least one enzyme of this invention, for example, the combinations of enzymes of the invention as set forth in Table 4, and discussed in Example 4, below; e.g., an exemplary mixture, “cocktail” or “enzyme ensemble” of the invention is: the exemplary enzymes SEQ ID NO:34, SEQ ID NO:360, SEQ ID NO:358, and SEQ ID NO:371; or, the exemplary enzymes SEQ ID NO:358, SEQ ID NO:360, SEQ ID NO:168; or, the exemplary enzymes SEQ ID NO:34, SEQ ID NO:360, SEQ ID NO:214; or, the exemplary enzymes SEQ ID NO:360, SEQ ID NO:90, SEQ ID NO:358; etc. as expressly set forth in Table 4.

The invention provides methods for processing a biomass material comprising lignocellulose comprising contacting a composition comprising a cellulose, a lignin, or a fermentable sugar with at least one polypeptide of the invention, or a polypeptide encoded by a nucleic acid of the invention, or an enzyme ensemble, product of manufacture or “cocktail” of the invention. In one aspect, the biomass material comprising lignocellulose is derived from an agricultural crop, is a byproduct of a food or a feed production, is a lignocellulosic waste product, or is a plant residue or a waste paper or waste paper product. In one aspect, enzyme of the invention has an activity comprising a glycosyl hydrolase, endoglucanase, cellobiohydrolase, beta-glucosidase, xylanase, mannanse, β-xylosidase and/or arabinofuranosidase activity. In one aspect, the plant residue comprise grain, seeds, stems, leaves, hulls, husks, corn or corn cobs, corn stover, hay, straw (e.g., a rice straw or a wheat straw, or any the dry stalk of any cereal plant) and/or grasses (e.g., Indian grass or switch grass). In one aspect, the grasses are Indian grass or switch grass, wood, wood chips, wood pulp and sawdust, or wood waste, and optionally the paper waste comprises discarded or used photocopy paper, computer printer paper, notebook paper, notepad paper, typewriter paper, newspapers, magazines, cardboard and paper-based packaging materials. In one aspect, the processing of the biomass material generates a biofuel, e.g., a bioalcohol such as bioethanol, biomethanol, biobutanol or biopropanol.

The invention provides dairy products comprising a polypeptide of the invention, or a polypeptide encoded by a nucleic acid of the invention, or an enzyme ensemble, product of manufacture or “cocktail” of the invention. In one aspect, the dairy product comprises a milk, an ice cream, a cheese or a yogurt. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides method for improving texture and flavor of a dairy product comprising the following steps: (a) providing a polypeptide of the invention, or a polypeptide encoded by a nucleic acid of the invention, or an enzyme ensemble, product of manufacture or “cocktail” of the invention; (b) providing a dairy product; and (c) contacting the polypeptide of step (a) and the dairy product of step (b) under conditions wherein the polypeptide of the invention can improve the texture or flavor of the dairy product.

The invention provides textiles or fabrics comprising a polypeptide of the invention, or a polypeptide encoded by a nucleic acid of the invention, or an enzyme ensemble, product of manufacture or “cocktail” of the invention, wherein optionally the textile or fabric comprises a cellulose-containing fiber. In one aspect, the polypeptide of the invention has a lignocellulosic activity, e.g., an activity comprising a glycosyl hydrolase, and/or cellulase, endoglucanase, cellobiohydrolase, beta-glucosidase, xylanase, mannanse, β-xylosidase and/or arabinofuranosidase activity.

The invention provides methods for treating solid or liquid animal waste products comprising the following steps: (a) providing a polypeptide of the invention, or a polypeptide encoded by a nucleic acid of the invention, or an enzyme ensemble, product of manufacture or “cocktail” of the invention; (b) providing a solid or a liquid animal waste; and (c) contacting the polypeptide of step (a) and the solid or liquid waste of step (b) under conditions wherein the protease can treat the waste. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides processed waste products comprising a polypeptide of the invention, or a polypeptide encoded by a nucleic acid of the invention, or an enzyme ensemble, product of manufacture or “cocktail” of the invention. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides disinfectants comprising a polypeptide having glucose oxidase and/or cellulase activity, wherein the polypeptide comprises a sequence of the invention, or a polypeptide encoded by a nucleic acid of the invention, or an enzyme ensemble, product of manufacture or “cocktail” of the invention. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides biodefense or bio-detoxifying agents comprising a polypeptide having a lignocellulosic activity, e.g., a cellulase activity, wherein the polypeptide comprises a sequence of the invention, or a polypeptide encoded by a nucleic acid of the invention, or an enzyme ensemble, product of manufacture or “cocktail” of the invention. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides compositions (including enzyme ensembles and products of manufacture of the invention) comprising a mixture of enzymes of the invention, e.g., hemicellulose- and cellulose-hydrolyzing enzymes of the invention, and a biomass material, wherein optionally the biomass material comprises a lignocellulosic material derived from an agricultural crop, or the biomass material is a byproduct of a food or a feed production, or the biomass material is a lignocellulosic waste product, or the biomass material is a plant residue or a waste paper or waste paper product, or the biomass material comprises a plant residue, and optionally the plant residue comprises grains, seeds, stems, leaves, hulls, husks, corn or corn cobs, corn stover, grasses, wherein optionally grasses are Indian grass or switch grass, hay or straw (e.g., a rice straw or a wheat straw, or any the dry stalk of any cereal plant), wood, wood chips, wood pulp, wood waste, and/or sawdust, and optionally the paper waste comprises discarded or used photocopy paper, computer printer paper, notebook paper, notepad paper, typewriter paper, newspapers, magazines, cardboard and paper-based packaging materials. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides methods for processing a biomass material comprising providing enzyme ensembles (“cocktails”) or products of manufacture of the invention, or a mixture of hemicellulose- and cellulose-hydrolyzing enzymes of the invention, wherein the cellulose-hydrolyzing enzymes comprise at least one endoglucanase, cellobiohydrolase I, cellobiohydrolase II and β-glucosidase; and the hemicellulose-hydrolyzing enzymes comprise at least one xylanase, β-xylosidase and arabinofuranosidase, and contacting the mixture of enzymes with the biomass material, wherein optionally the biomass material comprising lignocellulose is derived from an agricultural crop, is a byproduct of a food or a feed production, is a lignocellulosic waste product, or is a plant residue or a waste paper or waste paper product, and optionally the plant residue comprise grains, seeds, stems, leaves, hulls, husks, corn or corn cobs, corn stover, grasses, wherein optionally grasses are Indian grass or switch grass, hay or straw (e.g., a rice straw or a wheat straw, or any the dry stalk of any cereal plant), wood, wood waste, wood chips, wood pulp and/or sawdust, and optionally the paper waste comprises discarded or used photocopy paper, computer printer paper, notebook paper, notepad paper, typewriter paper, newspapers, magazines, cardboard and paper-based packaging materials, and optionally method further comprises processing the biomass material to generate a biofuel, e.g., a bioalcohol such as bioethanol, biomethanol, biobutanol or biopropanol, an alcohol and/or a sugar (a saccharide). In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse,β-xylosidase and/or arabinofuranosidase activity.

The invention provides methods for processing a biomass material comprising providing a mixture of enzymes of the invention (including enzyme ensembles (“cocktails”) or products of manufacture of the invention), and contacting the enzyme mixture with the biomass material, wherein optionally the biomass material comprising lignocellulose is derived from an agricultural crop, is a byproduct of a food or a feed production, is a lignocellulosic waste product, or is a plant residue or a waste paper or waste paper product, and optionally the plant residue comprise seeds, stems, leaves, hulls, husks, corn or corn cobs, corn stover, corn fiber, grasses (e.g. Indian grass or switch grass), hay, grains, straw (e.g. rice straw or wheat straw or any the dry stalk of any cereal plant), sugarcane bagasse, sugar beet pulp, citrus pulp, and citrus peels, wood, wood thinnings, wood chips, wood pulp, pulp waste, wood waste, wood shavings and sawdust, construction and/or demolition wastes and debris (e.g. wood, wood shavings and sawdust), and optionally the paper waste comprises discarded or used photocopy paper, computer printer paper, notebook paper, notepad paper, typewriter paper, newspapers, magazines, cardboard and paper-based packaging materials, and recycled paper materials. In addition, urban wastes, e.g. the paper fraction of municipal solid waste, municipal wood waste, and municipal green waste, along with other materials containing sugar, starch, and/or cellulose can be used. Optionally the processing of the biomass material generates a biofuel, e.g., a bioalcohol such as bioethanol, biomethanol, biobutanol or biopropanol. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides chimeric polypeptides comprising a first domain and at least a second domain, wherein the first domain comprises, or consists of, an enzyme of the invention, and the second domain comprises a heterologous sequence, e.g., a heterologous domain, such as a heterologous or modified carbohydrate binding domain or a heterologous or modified dockerin domain. In alternative embodiments, the carbohydrate binding domain or module (CBM) is a cellulose-binding module or a lignin-binding domain, and optionally the second domain appended approximate to the enzyme's catalytic domain. In one aspect, the CBM comprises, or consists of, a CBM of the invention. In alternative embodiments, the second domain comprises, or consists of, a heparin and/or fibronectin binding domain, such as a fibronectin type III domain, e.g., FN3, and the like.

In alternative embodiments, the second domain is appended approximate to the C-terminus of the enzyme's catalytic domain. In one aspect, the polypeptide of the invention has a lignocellulosic activity, e.g., an activity comprising a glycosyl hydrolase, cellulase, endoglucanase, cellobiohydrolase, beta-glucosidase, xylanase, mannanse, β-xylosidase and/or arabinofuranosidase activity.

The invention provides chimeric polypeptides comprising (a) a first domain and at least a second domain, wherein the first domain comprises, or consists of, an enzyme and/or a carbohydrate binding domain/ module (CBM) of the invention, and the second domain comprises, or consists of, a heterologous or modified carbohydrate binding domain (CBM), a heterologous or modified dockerin domain, a heterologous or modified prepro domain, or a heterologous or modified active site; (b) the chimeric polypeptide of (a), wherein the carbohydrate binding domain (CBM) comprises, or consists of, a cellulose-binding module or a lignin-binding domain; (c) the chimeric polypeptide of (a) or (b), wherein the CBM is approximate to the enzyme's catalytic domain; (d) the chimeric polypeptide of (a), (b) or (c), wherein the at least one CBM is positioned approximate to the polypeptide's catalytic domain; (e) the chimeric polypeptide of (d), wherein the at least one CBM is positioned: approximate to the C-terminus of the polypeptide's catalytic domain, or, approximate to the N-terminus of the polypeptide's catalytic domain, or both; (f) the chimeric polypeptide of any of (a), (b), (c) or (e), wherein the chimeric polypeptide comprises, or consists of, a recombinant chimeric protein.

The invention provides chimeric polypeptides comprising (a) a polypeptide of the invention having a lignocellulosic enzyme activity, and a domain comprising, or consisting of, at least one heterologous or modified carbohydrate binding domain-module (CBM) (e.g., a glycosyl hydrolase domain), or at least one internally rearranged CBM, or any combination thereof; (b) the chimeric polypeptide of (a), wherein the heterologous or modified or internally rearranged CBM comprises a CBM_{—}1, CBM_{—}2, CBM_{—}2a, CBM_{—}2b, CBM_{—}3, CBM_{—}3a, CBM_{—}3b, CBM_{—}3c, CBM_{—}4, CBM_{—}5, CBM_{—}5_{—}12, CBM_{—}6, CBM_{—}7, CBM_{—}8, CBM_{—}9, CBM_{—}10, CBM_{—}11, CBM_{—}12, CBM_{—}13, CBM_{—}14, CBM_{—}15, CBM_{—}16 or any of the CBMs from a CMB family of CBM_{—}1 to CBM_{—}48; a glycosyl hydrolase binding domain; a CBM of this invention (e.g., as described herein, CBMs of this invention also described in the Sequence Listing); or any combination thereof; (c) the chimeric polypeptide of (a) or (b), wherein the CBM comprises a cellulose-binding module or a lignin-binding domain; (d) the chimeric polypeptide of (a), (b) or (c), wherein the at least one CBM is positioned approximate to the polypeptide's catalytic domain; (e) the chimeric polypeptide of (d), wherein the at least one CBM is positioned: approximate to the C-terminus of the polypeptide's catalytic domain, or, approximate to the N-terminus of the polypeptide's catalytic domain, or both; or (f) the chimeric polypeptide of any of (a), (b), (c) or (e), wherein the chimeric polypeptide is a recombinant chimeric protein.

The invention provides isolated, synthetic and/or recombinant carbohydrate binding domain-modules (CBMs) comprising, or consisting of: (a) at least one CBM as set forth in Table 5, and the Sequence Listing; (b) at least one CBM as set forth in Table 6, and the Sequence Listing; or (c) a combination thereof. In alternative embodiments, carbohydrate binding domain-modules (CBMs) of the invention comprise, or consist of, any subsequence of any enzyme of this invention, including any subsequence of an exemplary enzyme of this invention, e.g., SEQ ID NO:2, SEQ ID NO:4, etc., wherein the subsequence comprises or consists of a CBM motif, e.g., a CBM_{—}1, CBM_{—}2, CBM_{—}2a, CBM_{—}2b, CBM_{—}3, CBM_{—}3a, CBM_{—}3b, CBM_{—}3c, CBM_{—}4, CBM_{—}5, CBM_{—}5_{—}12, CBM_{—}6, CBM_{—}7, CBM_{—}8, CBM_{—}9, CBM_{—}10, CBM_{—}11, CBM_{—}12, CBM_{—}13, CBM_{—}14, CBM_{—}15, CBM_{—}16 or any of the CBMs from a CMB family of CBM_{—}1 to CBM_{—}48.

The details of one or more aspects of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF DRAWINGS

The following drawings are illustrative of aspects of the invention and are not meant to limit the scope of the invention as encompassed by the claims.

FIG. 1 is a block diagram of a computer system.

FIG. 2 is a flow diagram illustrating one aspect of a process for comparing a new nucleotide or protein sequence with a database of sequences in order to determine the homology levels between the new sequence and the sequences in the database.

FIG. 3 is a flow diagram illustrating one aspect of a process in a computer for determining whether two sequences are homologous.

FIG. 4 is a flow diagram illustrating one aspect of an identifier process 300 for detecting the presence of a feature in a sequence.

FIG. 5 illustrates an exemplary sugarcane processing processes of the invention incorporating use of at least one enzyme, or enzyme mixture, of the invention, as described in detail, below; FIG. 5A illustrates an exemplary sugar to ethanol process incorporating use of at least one enzyme, or enzyme mixture, of the invention; FIG. 5B illustrates an exemplary process of the invention incorporating use of at least one enzyme, or enzyme mixture, of the invention; and, FIG. 5C illustrates an exemplary process of the invention—an overview of the a dry mill process—that can incorporate use of at least one enzyme, or enzyme mixture, of the invention.

FIG. 6 illustrates an exemplary protocol for identifying an enzyme of the invention: a glucose oxidase assay for quantifying glucose, as described in detail in Example 5, below.

FIG. 7 illustrates data summarizing the results of various exemplary mixtures' enzymatic activity under conditions comprising 37° C. digest on 0.1% AVICEL® substrate, as described in detail in Example 4, below.

FIG. 8 illustrates data summarizing the results of the various exemplary mixtures' enzymatic activity under conditions comprising 37° C. digest on 0.23% bagasse, as described in detail in Example 4, below.

FIG. 9A illustrates a standard curve from an exemplary B-glucosidase activity assay, as described in detail in Example 14, below. FIG. 9B shows how enzyme activity calculations for the exemplary β-glucosidase activity assay can be set up in EXCEL™, as described in detail in Example 14, below.

FIG. 10A illustrates a standard curve from an exemplary B-glucosidase activity assay, as described in detail in Example 14, below. FIG. 10B shows how enzyme activity calculations for the exemplary β-glucosidase activity assay can be set up in EXCEL™, as described in detail in Example 14, below.

FIG. 11 illustrates Table 1, showing data from the production and purification summary for beta-glucosidase enzymes of this invention, as described in detail in Example 14, below.

FIG. 12A illustrates a PAGE electrophoresis of the exemplary SEQ ID NO:548, SEQ ID NO:564, and SEQ ID NO:560 of this invention purified from supernatant and pellet cell fractions by the FPLC method, as described in detail in Example 14, below.

FIG. 12B illustrates a PAGE electrophoresis of SEQ ID NO:530 and SEQ ID NO:566 purified from supernatant and pellet cell fractions by the FPLC method, as described in detail in Example 14, below.

FIG. 13 illustrates a Table 7, and shows protein concentrations of purified beta-glucosidases of this invention determined by the three different methods, as described in detail in Example 14, below.

FIG. 14 illustrates a Table 8, and shows the specific activities of purified beta-glucosidases of this invention, as described in detail in Example 14, below.

FIG. 15 illustrates a Table 1, and shows the specific activity of exemplary beta-glucosidases of this invention, as described in detail in Example 14, below.

FIG. 16 illustrates data of the initial rate kinetics with enzyme dilutions selected empirically for each tested beta-glucosidase enzyme of this invention, as described in detail in Example 15, below.

FIG. 17 illustrates a PAGE electrophoresis with the exemplary SEQ ID NO:556, SEQ ID NO:560 of this invention, and A. niger beta-glucosidase, as described in detail in Example 15, below.

FIG. 18 illustrates data showing the hydrolysis of 2 mM cellobiose at different temperatures at pH 5 using exemplary enzymes of this invention, as described in detail in Example 15, below.

FIG. 19 illustrates data showing the hydrolysis of 2 mM cellobiose at different temperatures at pH 7 using exemplary enzymes of this invention, as described in detail in Example 15, below.

FIG. 20 illustrates an example arrangement for three sample preps, as described in detail in Example 17, below.

FIG. 21 is a table summarizing SPECTRAMAX™ data for an exemplary cellulase enzyme activity assay of the invention liberating 4-methylumbelliferone from MU-glucopyranoside, as described in detail in Example 17, below.

FIG. 22 is a table summarizing kinetic activity data for an exemplary cellulase enzyme activity assay of the invention, as described in detail in Example 17, below.

FIG. 23 illustrates data showing the wheat arabinoxylan digest products (digest profiles) of three enzymes that can be used in enzyme “cocktails” or mixtures of the invention, as described in detail in Example 20, below.

FIG. 24 is a graphic illustration of data showing how arabinofuranosidases of the invention synergize with xylanases of the invention to digest wheat arabinoxylan, as described in detail in Example 20, below.

FIG. 25 is a graphic illustration of data showing a promotion effect of beta (β)-xylosidases (as indicated in the figure) over the exemplary SEQ ID NO:719 xylanase in a wheat arabinoxylan digest, as described in detail in Example 20, below.

FIG. 26 is a graphic illustration of data showing a ferulic acid esterase activity with corn seed fiber as a substrate using an exemplary enzyme of this invention, as described in detail in Example 20, below.

FIG. 27 is a graphic illustration of data showing from an activity assay with acetylated xylan as a substrate using the exemplary acetyl xylan esterases of this invention SEQ ID NO:640, SEQ ID NO:650 and SEQ ID NO:688, as described in detail in Example 20, below.

FIG. 28 is a graphic illustration of data showing an alpha (α)-glucuronidase activity assay with an aldo-uronic acid mixture as a substrate using the exemplary acetyl xylan esterases of this invention SEQ ID NO:648, SEQ ID NO:654 and SEQ ID NO:680, as described in detail in Example 20, below.

Like reference symbols in the various drawings indicate like elements.

### DETAILED DESCRIPTION

In one aspect, the invention provides polypeptides having any lignocellulolytic (lignocellulosic) activity, including ligninolytic and cellulolytic activity, including, e.g., a glycosyl hydrolase, cellulase, endoglucanase, cellobiohydrolase, mannanase and/or β-glucosidase activity, polynucleotides encoding these polypeptides, and methods of making and using these polynucleotides and polypeptides. In one aspect, the invention provides polypeptides having a lignocellulosic activity, e.g., glucose oxidase activity, including enzymes that convert soluble oligomers to fermentable monomeric sugars in the saccharification of biomass. In one aspect, an activity of a polypeptide of the invention comprises enzymatic hydrolysis of (to degrade) soluble cellooligsaccharides and arabinoxylan oligomers into monomer xylose, arabinose and glucose. In one aspect, the invention provides thermostable and thermotolerant forms of polypeptides of the invention. The polypeptides of the invention can be used in a variety of pharmaceutical, agricultural and industrial contexts.

In one aspect, the invention provides a lignocellulosic enzyme, e.g., a glycosyl hydrolase, cellulase, endoglucanase, cellobiohydrolase, beta-glucosidase, xylanase, mannanse, β-xylosidase and/or arabinofuranosidase, with an increased catalytic rate, thus improving the process of substrate hydrolysis. In one aspect, the invention provides a lignocellulosic enzyme active under relatively extreme conditions, e.g., high or low temperatures or salt conditions, and/or acid or basic conditions, including pHs and temperatures higher or lower than physiologic. This increased efficiency in catalytic rate leads to an increased efficiency in producing sugars that, in one embodiment, are used by microorganisms for ethanol production. In one aspect, microorganisms generating enzyme of the invention are used with sugar hydrolyzing, e.g., ethanol-producing, microorganisms. Thus, the invention provides methods for biofuel, e.g., a bioalcohol such as bioethanol, biomethanol, biobutanol or biopropanol, production and making “clean fuels” based on alcohols, e.g., for transportation using biofuels.

In one aspect the invention provides compositions (e.g., enzyme preparations, feeds, drugs, dietary supplements) comprising the enzymes, polypeptides or polynucleotides of the invention. These compositions can be formulated in a variety of forms, e.g., as liquids, gels, pills, tablets, sprays, powders, food, feed pellets or encapsulated forms, including nanoencapsulated forms.

Assays for measuring cellulase activity, e.g., endoglucanase, cellobiohydrolase, beta-glucosidase, xylanase, mannanse, β-xylosidase and/or arabinofuranosidase activity, e.g., for determining if a polypeptide has cellulase activity, e.g., endoglucanase, cellobiohydrolase, beta-glucosidase, xylanase, mannanse, β-xylosidase and/or arabinofuranosidase activity, are well known in the art and are within the scope of the invention; see, e.g., Baker W L, Panow A, Estimation of cellulase activity using a glucose-oxidase-Cu(II) reducing assay for glucose, J Biochem Biophys Methods. 1991 Dec., 23(4):265-73; Sharrock K R, Cellulase assay methods: a review, J Biochem Biophys Methods. 1988 Oct., 17(2):81-105; Carder J H, Detection and quantitation of cellulase by Congo red staining of substrates in a cup-plate diffusion assay, Anal Biochem. 1986 Feb. 15, 153(1):75-9; Canevascini G., A cellulase assay coupled to cellobiose dehydrogenase, Anal Biochem. 1985 June, 147(2):419-27; Huang J S, Tang J, Sensitive assay for cellulase and dextranase. Anal Biochem. 1976 Jun., 73(2):369-77.

The pH of reaction conditions utilized by the invention is another variable parameter for which the invention provides. In certain aspects, the pH of the reaction is conducted in the range of about 3.0 or less to about 9.0 or more, and in one embodiment an enzyme of the invention is active under such acidic or basic conditions. In other aspects, a process of the invention is practiced at a pH of about 4.0, 4.5, 5.0, 5.5, 6.0, 6.5, 7.5, 8.0, 8.5, 9.0 or 9.5, or more, and in one embodiment an enzyme of the invention is active under such acidic or basic conditions. Reaction conditions conducted under alkaline conditions also can be advantageous, e.g., in some industrial or pharmaceutical applications of enzymes of the invention.

The invention provides compositions, including pharmaceuticals, additives and supplements, comprising a lignocellulosic enzyme of the invention, including polypeptides having glycosyl hydrolase, cellulase, endoglucanase, cellobiohydrolase, beta-glucosidase, xylanase, mannanse, β-xylosidase and/or arabinofuranosidase activity, in a variety of forms and formulations. In the methods of the invention, the lignocellulosic enzymes of the invention also are used in a variety of forms and formulations. For example, purified the lignocellulosic enzyme can be used in enzyme preparations deployed in a biofuel, e.g., a bioalcohol such as bioethanol, biomethanol, biobutanol or biopropanol, production or in pharmaceutical, food, feed or dietary aid applications. Alternatively, the enzymes of the invention can be used directly or indirectly in processes to produce a biofuel, e.g., a bioalcohol such as bioethanol, biomethanol, biobutanol or biopropanol, make clean fuels, process biowastes, process foods, chemicals, pharmaceuticals, supplements, liquids, foods or feeds, and the like.

Alternatively, the lignocellulosic enzyme, e.g., glycosyl hydrolase, cellulase, endoglucanase, cellobiohydrolase, beta-glucosidase, xylanase, mannanse, β-xylosidase and/or arabinofuranosidase polypeptides of the invention can be expressed in a microorganism (including bacterial, yeast, viruses, fungi and the like) using procedures known in the art. The microorganism expressing an enzyme of the invention can live on or in a plant, plant part (e.g., a seed) or an organism. In other aspects, the lignocellulosic enzyme of the invention can be immobilized on a solid support prior to use in the methods of the invention. Methods for immobilizing enzymes on solid supports are commonly known in the art, for example J. Mol. Cat. B: Enzymatic 6 (1999) 29-39; Chivata et al. Biocatalysis: Immobilized cells and enzymes, J Mol. Cat. 37 (1986) 1-24: Sharma et al., Immobilized Biomaterials Techniques and Applications, Angew. Chem. Int. Ed. Engl. 21 (1982) 837-54: Laskin (Ed.), Enzymes and Immobilized Cells in Biotechnology.

### Nucleic Acids, Probes and Inhibitory Molecules

The invention provides isolated, synthetic and recombinant nucleic acids, e.g., see Tables 1, 2, and 3, and the Examples, below, and the sequences of exemplary nucleic acids and polypeptides of the invention are set forth in the Sequence Listing; also describing exemplary nucleic acids encoding exemplary polypeptides of the invention, see e.g., Tables 1, 2, and 3, and Sequence Listing; including expression cassettes such as expression vectors, viruses, artificial chromosomes or any cloning vehicle, all comprising a nucleic acid of the invention.

In the sequence listing, for SEQ ID NOs:1-472, odd numbers represent nucleic acid protein-coding sequences and even number represent amino acid sequences. In reading the SEQ ID listing, in summary:
SEQ ID NOs:1-472: odd numbers represent nucleic acid protein-coding sequences and even numbers represent amino acid sequences;
SEQ ID NOs:473-479 represent amino acid sequences, SEQ ID NOs:480-488 represent nucleotide sequences;
SEQ ID NOs:489-700: odd numbers represent nucleic acid protein-coding sequences and even numbers represent amino acid sequences;
SEQ ID NOs:701-706 are linkers, all amino acid sequences;
SEQ ID NOs:707-717 are genomic, or gDNA, sequences for some of the enzymes initially derived from fungal sources (all nucleotides);
SEQ ID NOs:718-721: even numbers represent nucleotide sequences, odd numbers represent amino acid sequences).

For those sequences listed in Table 1A, which notes that SEQ ID NO:370, SEQ ID NO:373, SEQ ID NO:376, SEQ ID NO:379, SEQ ID NO:382, SEQ ID NO:385, SEQ ID NO:388, SEQ ID NO:391, SEQ ID NO:394, SEQ ID NO:397, SEQ ID NO:400, SEQ ID NO:403, SEQ ID NO:406, SEQ ID NO:409, SEQ ID NO:412, SEQ ID NO:415, SEQ ID NO:418 and SEQ ID NO:421 are exemplary enzyme coding, or cDNA sequences; and, SEQ ID NO:369, SEQ ID NO:372, SEQ ID NO:375, SEQ ID NO:378, SEQ ID NO:381, SEQ ID NO:384, SEQ ID NO:387, SEQ ID NO:390, SEQ ID NO:393, SEQ ID NO:396, SEQ ID NO:399, SEQ ID NO:402, SEQ ID NO:405, SEQ ID NO:408, SEQ ID NO:411, SEQ ID NO:414, SEQ ID NO:417 and SEQ ID NO:420, are exemplary genomic (or “gDNA”) sequences; and, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419 and SEQ ID NO:422, are exemplary protein (amino acid) sequences.

In summary:

TABLE 1A

| | gDNA SEQ ID | predicted cDNA | predicted protein |
|---|---|---|---|
| SEQ ID NO: | NO: | SEQ ID NO: | SEQ ID NO: |
| 369-371 | 369 | 370 | 371 |
| 372-374 | 372 | 373 | 374 |
| 375-377 | 375 | 376 | 377 |
| 378-380 | 378 | 379 | 380 |
| 381-383 | 381 | 382 | 383 |
| 384-386 | 384 | 385 | 386 |
| 387-389 | 387 | 388 | 389 |
| 390-392 | 390 | 391 | 392 |
| 393-395 | 393 | 394 | 395 |
| 396-398 | 396 | 397 | 398 |
| 399-401 | 399 | 400 | 401 |
| 402-404 | 402 | 403 | 404 |
| 405-407 | 405 | 406 | 407 |
| 408-410 | 408 | 409 | 410 |
| 411-413 | 411 | 412 | 413 |
| 414-416 | 414 | 415 | 416 |
| 417-419 | 417 | 418 | 419 |
| 420-422 | 420 | 421 | 422 |

TABLE 1B

| | | predicted | predicted |
|---|---|---|---|
| | gDNA SEQ | cDNA | protein |
| SEQ ID NOs: | ID NO: | SEQ ID NO: | SEQ ID NO: |
| 493, 494 | 707 | 493 | 494 |
| 495, 496 | 710 | 495 | 496 |
| 497, 498 | 711 | 497 | 498 |
| 499, 500 | 712 | 499 | 500 |
| 501, 502 | 713 | 501 | 502 |
| 503, 504 | 714 | 503 | 504 |
| 505, 506 | 715 | 505 | 506 |
| 507, 508 | 716 | 507 | 508 |
| 509, 510 | 717 | 509 | 510 |
| 511, 512 | 708 | 511 | 512 |
| 513, 514 | 709 | 513 | 514 |

The sequences listed in Table 1A and 1B, above, were initially derived from fungal sources, i.e., these exemplary sequences of the invention are fungal-derived nucleic acids and enzymes.

Tables 2 and 3, below are charts describing selected characteristics, including enzymatic activity, of exemplary nucleic acids and polypeptides of the invention, including sequence identity comparison of the exemplary sequences to public databases to identify activity of enzymes of the invention by homology (sequence identity) analysis. All sequences described in Tables 2 and 3 (all the exemplary sequences of the invention) have been subject to a BLAST search (as described in detail, below) against two sets of databases. The first database set is available through NCBI (National Center for Biotechnology Information). All results from searches against these databases are found in the columns entitled “NR Description”, “NR Accession Code”, “NR Evalue” or “NR Organism”. “NR” refers to the Non-Redundant nucleotide database maintained by NCBI. This database is a composite of GenBank, GenBank updates, and EMBL updates. The entries in the column “NR Description” refer to the definition line in any given NCBI record, which includes a description of the sequence, such as the source organism, gene name/protein name, or some description of the function of the sequence—thus identifying an activity of the listed exemplary enzymes of the invention by homology (sequence identity) analysis. The entries in the column “NR Accession Code” refer to the unique identifier given to a sequence record. The entries in the column “NR Evalue” refer to the Expect value (Evalue), which represents the probability that an alignment score as good as the one found between the query sequence (the sequences of the invention) and a database sequence would be found in the same number of comparisons between random sequences as was done in the present BLAST search. The entries in the column “NR Organism” refer to the source organism of the sequence identified as the closest BLAST (sequence homology) hit. The second set of databases is collectively known as the GENESEQ™ database, which is available through Thomson Derwent (Philadelphia, Pa.). All results from searches against this database are found in the columns entitled “GENESEQ™ Protein Description”, “GENESEQ™ Protein Accession Code”, “GENESEQ™ Protein Evalue”, “GENESEQ™ DNA Description”, “GENESEQ™ DNA Accession Code” or “GENESEQ™ DNA Evalue”. The information found in these columns is comparable to the information found in the NR columns described above, except that it was derived from BLAST searches against the GENESEQ™ database instead of the NCBI databases. In addition, this table includes the column “Predicted EC No.”. An EC number is the number assigned to a type of enzyme according to a scheme of standardized enzyme nomenclature developed by the Enzyme Commission of the Nomenclature Committee of the International Union of Biochemistry and Molecular Biology (IUBMB). The results in the “Predicted EC No.” column are determined by a BLAST search against the Kegg (Kyoto Encyclopedia of Genes and Genomes) database. If the top BLAST match has an Evalue equal to or less than e⁻⁶, the EC number assigned to the top match is entered into the table. The EC number of the top hit is used as a guide to what the EC number of the sequence of the invention might be. The columns “Query DNA Length” and “Query Protein Length” refer to the number of nucleotides or the number amino acids, respectively, in the sequence of the invention that was searched or queried against either the NCBI or GENESEQ™ databases. The columns “GENESEQ™ or NR DNA Length” and “GENESEQ™ or NR Protein Length” refer to the number of nucleotides or the number amino acids, respectively, in the sequence of the top match from the BLAST search. The results provided in these columns are from the search that returned the lower Evalue, either from the NCBI databases or the Geneseq database. The columns “GENESEQ™ or NR % ID Protein” and “GENESEQ™ or NR % ID DNA” refer to the percent sequence identity between the sequence of the invention and the sequence of the top BLAST match. The results provided in these columns are from the search that returned the lower Evalue, either from the NCBI databases or the GENESEQ™ database.

Activity of exemplary sequences of the invention are listed in, inter alia, Tables 2 and 3, below (see also Tables 4 and 5, which lists exemplary enzyme mixtures, and CBMs, of the invention, respectively). To further aid in reading the tables, for example, in the first row of Table 2, labeled “SEQ ID NO:”, the numbers 369-371 represent the exemplary polypeptide of the invention having a sequence as set forth in SEQ ID NO:371, encoded by, e.g., SEQ ID NO:369 (this is a genomic sequence, as explained above); the “enzyme activity by homology” is the enzyme's activity assignment based on a top (closest) BLAST hit; the “enzyme activity by experiment” is the enzyme's activity in a broad interpretation as determined by experimental protocol; the “GH family” indicates the glycosyl hydrolase family of the listed exemplary enzyme; the “activity on PASC” is an experimentally determined level of activity of the listed enzyme on the substrate phosphoric acid swollen cellulose (PASC), as described below; the “Signalp Cleavage Site” is the listed exemplary enzyme's signal sequence (or “signal peptide”, or SP), as determined by the paradigm Signalp, as discussed below (see Nielsen (1997), infra); the “Predicted Signal Sequence” is listed from the amino terminal to the carboxy terminal, for example, for the polypeptide SEQ ID NO:38 in the second row of Table 2, the signal peptide is “MVKSRKISILLAVAMLVSIMIPTTAFA”; the “source” is the microorganism source from which the exemplary nucleic acid and polypeptide of the invention was first derived.

TABLE 2

| | | | Activity on | Predicted |
|---|---|---|---|---|
| SEQ ID NO: | Activity | GH Family | PASC? | EC Number |
| 1, 2 | Glycosidase | 6 | Yes | 3.2.1.91 |
| 101, 102 | Glycosidase | 48 | Yes | |
| 103, 104 | Glycosidase | 5 | Yes | 3.2.1.4 |
| 105, 106 | Glycosidase | 5 | Yes | 3.2.1.4 |
| 107, 108 | Glycosidase | 45 | Yes | |
| 109, 110 | Glycosidase | 5 | Yes | 3.2.1.4 |
| 11, 12 | Glycosidase | 6 | Yes | 3.2.1.91 |
| 111, 112 | Glycosidase | 5 | Yes | 3.2.1.4 |
| 113, 114 | Glycosidase | 5 | Yes | 3.2.1.4 |
| 115, 116 | Glycosidase | 48 | No | 3.2.1.4 |
| 117, 118 | Glycosidase | 5 | Yes | 3.2.1.4 |
| 119, 120 | Glycosidase | 5 | Yes | 3.2.1.4 |
| 121, 122 | Glycosidase | 5 | Yes | 3.2.1.4 |
| 123, 124 | Glycosidase | 3 | No | 3.2.1.21 |
| 125, 126 | Glycosidase | 5 | Yes | 3.2.1.4 |
| 127, 128 | Glycosidase | 5 | Yes | 3.2.1.4 |
| 129, 130 | Glycosidase | 5 | Yes | 3.2.1.4 |
| 13, 14 | Glycosidase | 6 | Yes | |
| 131, 132 | Glycosidase | 5 | Yes | 3.2.1.4 |
| 133, 134 | Glycosidase | 5 | Yes | 3.2.1.4 |
| 135, 136 | Glycosidase | 48 | Yes | |
| 137, 138 | Glycosidase | 48 | Yes | 3.2.1.8 |
| 139, 140 | Glycosidase | 48 | Yes | |
| 141, 142 | Glycosidase | 5 | Yes | 3.2.1.4 |
| 143, 144 | Glycosidase | 5 | Yes | 3.2.1.4 |
| 145, 146 | Glycosidase | 9 | Yes | |
| 147, 148 | Glycosidase | 5 | Yes | 3.2.1.4 |
| 149, 150 | Glycosidase | 5 | Yes | 3.2.1.4 |
| 15, 16 | Glycosidase | 6 | Yes | 3.2.1.91 |
| 151, 152 | Glycosidase | 9 | Yes | |
| 153, 154 | Glycosidase | 5 | Yes | 3.2.1.4 |
| 155, 156 | Glycosidase | 9 | Yes | 3.2.1.4 |
| 157, 158 | Glycosidase | 5 | Yes | 3.2.1.4 |
| 159, 160 | Glycosidase | 5 | Yes | 3.2.1.4 |
| 161, 162 | Glycosidase | 45 | Yes | 3.2.1.3 |
| 163, 164 | Glycosidase | 6 | Yes | 3.2.1.91 |
| 165, 166 | Glycosidase | 6 | Yes | 3.2.1.91 |
| 167, 168 | Glycosidase | 5 | Yes | 3.2.1.4 |
| 169, 170 | Glycosidase | 48 | Yes | 3.2.1.4 |
| 17, 18 | Glycosidase | 48 | Yes | |
| 171, 172 | Glycosidase | 48 | Yes | |
| 173, 174 | Glycosidase | 48 | Yes | 3.2.1.4 |
| 175, 176 | Glycosidase | 48 | Yes | 3.2.1.4 |
| 177, 178 | Glycosidase | 48 | Yes | 3.2.1.4 |
| 179, 180 | Glycosidase | 48 | Yes | 3.2.1.3 |
| 181, 182 | Glycosidase | 6 | Yes | 3.2.1.91 |
| 183, 184 | Glycosidase | 6 | Yes | 3.2.1.91 |
| 185, 186 | Glycosidase | 6 | Yes | 3.2.1.91 |
| 187, 188 | Glycosidase | 48 | Yes | |
| 189, 190 | Glycosidase | 48 | Yes | 3.2.1.3 |
| 19, 20 | Glycosidase | 6 | No | 3.2.1.91 |
| 191, 192 | Glycosidase | 6 | Yes | 3.2.1.91 |
| 193, 194 | Glycosidase | 48 | Yes | 3.2.1.4 |
| 195, 196 | Glycosidase | 48 | Yes | 3.2.1.8 |
| 197, 198 | Glycosidase | 6 | No | |
| 199, 200 | Glycosidase | 6 | No | |
| 201, 202 | Glycosidase | 6 | Yes | 3.2.1.91 |
| 203, 204 | Glycosidase | 6 | Yes | 3.2.1.91 |
| 205, 206 | Glycosidase | 9 | No | 3.2.1.4 |
| 207, 208 | Glycosidase | 48 | Yes | |
| 209, 210 | Glycosidase | 6 | Yes | 3.2.1.91 |
| 21, 22 | Glycosidase | 5 | Yes | 3.2.1.4 |
| 211, 212 | Glycosidase | 9 | Yes | 3.2.1.4 |
| 213, 214 | Glycosidase | 5 | Yes | 3.2.1.4 |
| 215, 216 | Glycosidase | 6 | No | 3.2.1.91 |
| 217, 218 | Glycosidase | 6 | Yes | |
| 219, 220 | Glycosidase | 6 | Yes | |
| 221, 222 | Glycosidase | 6 | Yes | 3.2.1.91 |
| 223, 224 | Glycosidase | 6 | Yes | 3.2.1.91 |
| 225, 226 | Glycosidase | 6 | Yes | 3.2.1.91 |
| 227, 228 | Glycosidase | 6 | No | 3.2.1.91 |
| 229, 230 | Glycosidase | 6 | Yes | 3.2.1.91 |
| 23, 24 | Glycosidase | 6 | Yes | |
| 231, 232 | Glycosidase | 9 | Yes | |
| 233, 234 | Glycosidase | 5 | Yes | 3.2.1.4 |
| 235, 236 | Glycosidase | 6 | Yes | |
| 237, 238 | Glycosidase | 6 | No | 3.2.1.91 |
| 239, 240 | Glycosidase | 6 | Yes | |
| 241, 242 | Glycosidase | 48 | Yes | 3.2.1.8 |
| 243, 244 | Glycosidase | 6 | Yes | 3.2.1.91 |
| 245, 246 | Glycosidase | 9 | Yes | 3.2.1.4 |
| 247, 248 | Glycosidase | 9 | Yes | 3.2.1.4 |
| 249, 250 | Glycosidase | 5 | Yes | 3.2.1.4 |
| 25, 26 | Glycosidase; GH family 6 (cellulase) | 6 | Yes | 3.2.1.91 |
| 251, 252 | Glycosidase | 45 | Yes | 3.2.1.3 |
| 253, 254 | Glycosidase | 48 | No | 3.2.1.4 |
| 255, 256 | Glycosidase | 48 | No | 3.2.1.4 |
| 257, 258 | Glycosidase | 48 | No | 3.2.1.4 |
| 259, 260 | Glycosidase | 48 | No | 3.2.1.4 |
| 261, 262 | Glycosidase | 5 | Yes | 3.2.1.4 |
| 263, 264 | Glycosidase | 48 | Yes | 3.2.1.4 |
| 265, 266 | Glycosidase | | No | |
| 267, 268 | Glycosidase | 6 | Yes | 3.2.1.91 |
| 269, 270 | Glycosidase | 5 | No | 3.2.1.4 |
| 27, 28 | Glycosidase | 7 | No | |
| 271, 272 | Glycosidase | 5 | Yes | 3.2.1.4 |
| 273, 274 | Glycosidase | 5 | Yes | 3.2.1.4 |
| 275, 276 | Glycosidase | 5 | Yes | 3.2.1.4 |
| 277, 278 | Glycosidase | 5 | No | 3.2.1.4 |
| 279, 280 | Glycosidase | 5 | Yes | 3.2.1.4 |
| 281, 282 | Glycosidase | 6 | Yes | 3.2.1.91 |
| 283, 284 | Glycosidase | 5 | Yes | 3.2.1.4 |
| 285, 286 | Glycosidase | 5 | No | 3.2.1.4 |
| 287, 288 | Glycosidase | 5 | Yes | 3.2.1.4 |
| 289, 290 | Glycosidase | 5 | Yes | 3.2.1.4 |
| 29, 30 | Glycosidase | 7 | No | |
| 291, 292 | Glycosidase | 9 | Yes | 3.2.1.4 |
| 293, 294 | Glycosidase | 9 | Yes | 3.2.1.4 |
| 295, 296 | Glycosidase | 9 | No | 3.2.1.4 |
| 297, 298 | Glycosidase | 9 | No | 3.2.1.4 |
| 299, 300 | Glycosidase | 9 | No | 3.2.1.4 |
| 3, 4 | Glycosidase | 6 | Yes | 3.2.1.91 |
| 301, 302 | Glycosidase | 9 | No | 3.2.1.4 |
| 303, 304 | Glycosidase | 9 | Yes | 3.2.1.4 |
| 305, 306 | Glycosidase | 5 | Yes | 3.2.1.4 |
| 307, 308 | Glycosidase | 5 | Yes | 3.2.1.4 |
| 309, 310 | Glycosidase | 9 | No | 3.2.1.4 |
| 31, 32 | Glycosidase | 7 | Yes | |
| 311, 312 | Glycosidase | 5 | Yes | 3.2.1.4 |
| 313, 314 | Glycosidase | 45 | No | |
| 315, 316 | Glycosidase | 6 | No | 3.2.1.91 |
| 317, 318 | Glycosidase | 6 | No | 3.2.1.91 |
| 319, 320 | Glycosidase | 6 | No | 3.2.1.91 |
| 321, 322 | Glycosidase | 6 | No | 3.2.1.91 |
| 323, 324 | Glycosidase | 6 | No | |
| 325, 326 | Glycosidase | 6 | No | |
| 327, 328 | Glycosidase | 6 | No | |
| 329, 330 | Glycosidase | 6 | No | |
| 33, 34 | Glycosidase; Cellobiohydrolase | 7 | Yes | |
| 331, 332 | Glycosidase | 6 | No | |
| 333, 334 | Glycosidase | 6 | No | 3.2.1.91 |
| 335, 336 | Glycosidase | 6 | No | 3.2.1.91 |
| 337, 338 | Glycosidase | 9 | No | 3.2.1.4 |
| 339, 340 | Glycosidase | 9 | No | |
| 341, 342 | Glycosidase | 6 | No | 3.2.1.91 |
| 343, 344 | Glycosidase | 6 | No | 3.2.1.91 |
| 345, 346 | Glycosidase | 6 | No | 3.2.1.91 |
| 347, 348 | Glycosidase | 45 | | |
| 349, 350 | Glycosidase | 6 | | 3.2.1.91 |
| 35, 36 | Glycosidase | 6 | Yes | 3.2.1.91 |
| 351, 352 | Glycosidase | 6 | | 3.2.1.91 |
| 353, 354 | Glycosidase | 6 | Yes | 3.2.1.91 |
| 355, 356 | Glycosidase | 7 | Yes | |
| 357, 358 | Glycosidase | 6 | Yes | 3.2.1.91 |
| 359, 360 | Glycosidase; Cellobiohydrolase | 7 | Yes | |
| 361, 362 | Glycosidase | 9 | Yes | |
| 363, 364 | Glycosidase | 8 | Yes | 3.2.1.14 |
| 365, 366 | Glycosidase | 8 | Yes | |
| 367, 368 | Glycosidase | 9 | Yes | |
| 369-371 | | 7 | Yes | |
| 37, 38 | Glycosidase | 48 | Yes | |
| 372-374 | | 6 | No | |
| 375-377 | | 6 | Yes | |
| 378-380 | | 6 | Yes | |
| 381-383 | | 6 | Yes | |
| 384-386 | | 6 | No | |
| 387-389 | | 6 | No | |
| 39, 40 | Glycosidase | 48 | Yes | 3.2.1.4 |
| 390-392 | | 6 | Yes | |
| 393-395 | | 6 | Yes | |
| 396-398 | | 6 | Yes | |
| 399-401 | | 6 | Yes | |
| 402-404 | | 6 | No | |
| 405-407 | | 6 | Yes | |
| 408-410 | | 6 | Yes | |
| 41, 42 | Glycosidase | 5 | Yes | 3.2.1.4 |
| 411-413 | | 6 | Yes | |
| 414-416 | | 6 | No | |
| 417-419 | | 6 | Yes | |
| 420-422 | | 6 | Yes | |
| 423, 424 | β-glucosidase | | | 3.2.1.21 |
| 425, 426 | | | | 3.2.1.4 |
| 427, 428 | Alkaline endoglucanase/cellulase | | | 3.2.1.4 |
| 429, 430 | | | | 3.2.1.4 |
| 43, 44 | Glycosidase | 9 | Yes | 3.2.1.4 |
| 431, 432 | Glycosidase | | | 3.2.1.8 |
| 433, 434 | Glycosidase | | | 3.2.1.8 |
| 435, 436 | Glycosidase | | | 3.2.1. |
| 437, 438 | Glycosidase | | | 3.2.1.4 |
| 439, 440 | Glycosidase | | | 3.2.1.8 |
| 441, 442 | Glycosidase | | | 3.2.1.8 |
| 443, 444 | Glycosidase | | | 3.2.1.8 |
| 445, 446 | Glycosidase | | | |
| 447, 448 | Glycosidase | | | 3.2.1.4 |
| 449, 450 | | | | 3.2.1.8 |
| 45, 46 | Glycosidase | | Yes | 3.2.1.55 |
| 451, 452 | Esterase | | | |
| 453, 454 | Glycosidase | | | |
| 455, 456 | Binding | | | |
| 457, 458 | Binding | | | |
| 459, 460 | | | | |
| 461, 462 | Glycosidase | | | 3.2.1. |
| 463, 464 | | | | 3.2.1.4 |
| 465, 466 | | | | 3.2.1.4 |
| 467, 468 | | | | 3.2.1.8 |
| 469, 470 | | | | 3.2.1.4 |
| 47, 48 | Glycosidase | 9 | Yes | 3.2.1.4 |
| 471, 472 | | | | 3.2.1.4 |
| 49, 50 | Glycosidase | 5 | Yes | 3.2.1.4 |
| 5, 6 | Glycosidase | 6 | Yes | 3.2.1.8 |
| 51, 52 | Glycosidase | 9 | Yes | |
| 53, 54 | Glycosidase | 5 | Yes | 3.2.1.4 |
| 55, 56 | Glycosidase | 5 | Yes | 3.2.1.4 |
| 57, 58 | Glycosidase | 9 | Yes | 3.2.1.4 |
| 59, 60 | Glycosidase | 45 | Yes | |
| 61, 62 | Glycosidase | 9 | Yes | |
| 63, 64 | Glycosidase | 9 | Yes | 3.2.1.4 |
| 65, 66 | Glycosidase | 5 | Yes | 3.2.1.4 |
| 67, 68 | Glycosidase | 5 | Yes | 3.2.1.4 |
| 69, 70 | Glycosidase | 5 | No | 3.2.1.4 |
| 7, 8 | Glycosidase | 5 | Yes | 3.2.1.4 |
| 71, 72 | Glycosidase | 45 | Yes | |
| 73, 74 | Glycosidase | 5 | Yes | 3.2.1.4 |
| 75, 76 | Glycosidase | 9 | Yes | 3.2.1.4 |
| 77, 78 | Glycosidase | 5 | Yes | 3.2.1.4 |
| 79, 80 | Glycosidase | 5 | Yes | 3.2.1.4 |
| 81, 82 | Glycosidase | | Yes | 3.2.1.55 |
| 83, 84 | Glycosidase | 5 | Yes | 3.2.1.4 |
| 85, 86 | Glycosidase | 9 | Yes | 3.2.1.4 |
| 87, 88 | Glycosidase | 5 | Yes | 3.2.1.4 |
| 89, 90 | Glycosidase | 5 | Yes | 3.2.1.4 |
| 9, 10 | Glycosidase | 5 | Yes | 3.2.1.4 |
| 91, 92 | Glycosidase | 48 | Yes | |
| 93, 94 | Glycosidase | 48 | Yes | 3.2.1.4 |
| 95, 96 | Glycosidase | 48 | Yes | 3.2.1.4 |
| 97, 98 | Glycosidase | 48 | Yes | |
| 99, 100 | Glycosidase | 48 | Yes | |

| | | GH | |
|---|---|---|---|
| SEQ ID NO: | Activity | Family | Signalp Cleavage Site |
| 473 | Glycine max glycinin GY1 | | |
| | signal sequence | | |
| 474 | ER retention sequence | | |
| 475 | sporamin vacuolar | | |
| | targeting sequence | | |
| 476 | transit peptide from | | |
| | ferredoxin-NADP+ | | |
| | reductase (FNR) of | | |
| | Cyanophora paradoxa | | |
| 477 | protein storage vacuole | | |
| | (PSV) sequence from b- | | |
| | conglycinin | | |
| 478 | gamma zein 27 kD signal | | |
| | sequence | | |
| 479 | vacuole sequence domain | | |
| | (VSD) from barley | | |
| | polyamine oxidase | | |
| 480 | dicot optimized SEQ ID | | |
| | NO: 359 | | |
| 481 | dicot optimized SEQ ID | | |
| | NO: 357 | | |
| 482 | dicot optimized SEQ ID | | |
| | NO: 167 | | |
| 483 | monocot optimized SEQ | | |
| | ID NO: 359 | | |
| 484 | monocot optimized SEQ | | |
| | ID NO: 357 | | |
| 485 | monocot optimized SEQ | | |
| | ID NO: 167 | | |
| 486 | monocot optimized SEQ | | |
| | ID NO: 33 | | |
| 487 | dicot optimized SEQ ID | | |
| | NO: 33 | | |
| 488 | Cestrum yellow leaf curl | | |
| | virus promoter plus leader | | |
| 701 | linker | | |
| 702 | linker | | |
| 703 | linker | | |
| 704 | linker | | |
| 705 | linker | | |
| 706 | linker | | |
| 1, 2 | Glycosidase | 6 | Probability: 1.000 AA1: 33 AA2: 34 |
| 101, 102 | Glycosidase | 48 | Probability: 0.889 AA1: 19 AA2: 20 |
| 103, 104 | Glycosidase | 5 | Probability: 1.000 AA1: 24 AA2: 25 |
| 105, 106 | Glycosidase | 5 | |
| 107, 108 | Glycosidase | 45 | Probability: 1.000 AA1: 21 AA2: 22 |
| 109, 110 | Glycosidase | 5 | |
| 11, 12 | Glycosidase | 6 | Probability: 1.000 AA1: 30 AA2: 31 |
| 111, 112 | Glycosidase | 5 | Probability: 0.819 AA1: 18 AA2: 19 |
| 113, 114 | Glycosidase | 5 | |
| 115, 116 | Glycosidase | 48 | |
| 117, 118 | Glycosidase | 5 | |
| 119, 120 | Glycosidase | 5 | |
| 121, 122 | Glycosidase | 5 | |
| 123, 124 | Glycosidase | 3 | |
| 125, 126 | Glycosidase | 5 | |
| 127, 128 | Glycosidase | 5 | |
| 129, 130 | Glycosidase | 5 | |
| 13, 14 | Glycosidase | 6 | Probability: 1.000 AA1: 24 AA2: 25 |
| 131, 132 | Glycosidase | 5 | Probability: 1.000 AA1: 23 AA2: 24 |
| 133, 134 | Glycosidase | 5 | |
| 135, 136 | Glycosidase | 48 | Probability: 1.000 AA1: 32 AA2: 33 |
| 137, 138 | Glycosidase | 48 | Probability: 1.000 AA1: 37 AA2: 38 |
| 139, 140 | Glycosidase | 48 | Probability: 1.000 AA1: 38 AA2: 39 |
| 141, 142 | Glycosidase | 5 | |
| 143, 144 | Glycosidase | 5 | Probability: 1.000 AA1: 25 AA2: 26 |
| 145, 146 | Glycosidase | 9 | Probability: 1.000 AA1: 31 AA2: 32 |
| 147, 148 | Glycosidase | 5 | Probability: 0.998 AA1: 31 AA2: 32 |
| 149, 150 | Glycosidase | 5 | Probability: 0.997 AA1: 22 AA2: 23 |
| 15, 16 | Glycosidase | 6 | |
| 151, 152 | Glycosidase | 9 | Probability: 1.000 AA1: 32 AA2: 33 |
| 153, 154 | Glycosidase | 5 | |
| 155, 156 | Glycosidase | 9 | Probability: 1.000 AA1: 41 AA2: 42 |
| 157, 158 | Glycosidase | 5 | |
| 159, 160 | Glycosidase | 5 | Probability: 1.000 AA1: 28 AA2: 29 |
| 161, 162 | Glycosidase | 45 | Probability: 1.000 AA1: 21 AA2: 22 |
| 163, 164 | Glycosidase | 6 | Probability: 1.000 AA1: 24 AA2: 25 |
| 165, 166 | Glycosidase | 6 | Probability: 1.000 AA1: 30 AA2: 31 |
| 167, 168 | Glycosidase; | 5 | Probability: 0.829 AA1: 18 AA2: 19 |
| | Endoglucanase | | |
| 169, 170 | Glycosidase | 48 | Probability: 1.000 AA1: 52 AA2: 53 |
| 17, 18 | Glycosidase | 48 | Probability: 1.000 AA1: 37 AA2: 38 |
| 171, 172 | Glycosidase | 48 | Probability: 1.000 AA1: 28 AA2: 29 |
| 173, 174 | Glycosidase | 48 | |
| 175, 176 | Glycosidase | 48 | |
| 177, 178 | Glycosidase | 48 | Probability: 0.998 AA1: 37 AA2: 38 |
| 179, 180 | Glycosidase | 48 | Probability: 1.000 AA1: 38 AA2: 39 |
| 181, 182 | Glycosidase | 6 | Probability: 1.000 AA1: 69 AA2: 70 |
| 183, 184 | Glycosidase | 6 | Probability: 1.000 AA1: 45 AA2: 46 |
| 185, 186 | Glycosidase | 6 | Probability: 0.995 AA1: 41 AA2: 42 |
| 187, 188 | Glycosidase | 48 | |
| 189, 190 | Glycosidase | 48 | Probability: 1.000 AA1: 38 AA2: 39 |
| 19, 20 | Glycosidase | 6 | Probability: 0.996 AA1: 23 AA2: 24 |
| 191, 192 | Glycosidase | 6 | |
| 193, 194 | Glycosidase | 48 | |
| 195, 196 | Glycosidase | 48 | Probability: 1.000 AA1: 37 AA2: 38 |
| 197, 198 | Glycosidase | 6 | Probability: 1.000 AA1: 18 AA2: 19 |
| 199, 200 | Glycosidase | 6 | Probability: 1.000 AA1: 18 AA2: 19 |
| 201, 202 | Glycosidase | 6 | Probability: 0.943 AA1: 19 AA2: 20 |
| 203, 204 | Glycosidase | 6 | Probability: 0.943 AA1: 19 AA2: 20 |
| 205, 206 | Glycosidase | 9 | Probability: 0.830 AA1: 31 AA2: 32 |
| 207, 208 | Glycosidase | 48 | Probability: 1.000 AA1: 26 AA2: 27 |
| 209, 210 | Glycosidase | 6 | Probability: 1.000 AA1: 29 AA2: 30 |
| 21, 22 | Glycosidase | 5 | Probability: 1.000 AA1: 28 AA2: 29 |
| 211, 212 | Glycosidase | 9 | |
| 213, 214 | Glycosidase | 5 | |
| 215, 216 | Glycosidase | 6 | Probability: 0.965 AA1: 29 AA2: 30 |
| 217, 218 | Glycosidase | 6 | Probability: 0.998 AA1: 38 AA2: 39 |
| 219, 220 | Glycosidase | 6 | Probability: 0.998 AA1: 38 AA2: 39 |
| 221, 222 | Glycosidase | 6 | Probability: 1.000 AA1: 30 AA2: 31 |
| 223, 224 | Glycosidase | 6 | Probability: 1.000 AA1: 30 AA2: 31 |
| 225, 226 | Glycosidase | 6 | Probability: 1.000 AA1: 23 AA2: 24 |
| 227, 228 | Glycosidase | 6 | Probability: 1.000 AA1: 30 AA2: 31 |
| 229, 230 | Glycosidase | 6 | Probability: 1.000 AA1: 30 AA2: 31 |
| 23, 24 | Glycosidase | 6 | Probability: 0.983 AA1: 33 AA2: 34 |
| 231, 232 | Glycosidase | 9 | Probability: 1.000 AA1: 22 AA2: 23 |
| 233, 234 | Glycosidase | 5 | Probability: 1.000 AA1: 22 AA2: 23 |
| 235, 236 | Glycosidase | 6 | Probability: 0.999 AA1: 37 AA2: 38 |
| 237, 238 | Glycosidase | 6 | Probability: 0.999 AA1: 37 AA2: 38 |
| 239, 240 | Glycosidase | 6 | Probability: 0.999 AA1: 37 AA2: 38 |
| 241, 242 | Glycosidase | 48 | Probability: 0.985 AA1: 28 AA2: 29 |
| 243, 244 | Glycosidase | 6 | Probability: 0.992 AA1: 19 AA2: 20 |
| 245, 246 | Glycosidase | 9 | Probability: 1.000 AA1: 33 AA2: 34 |
| 247, 248 | Glycosidase | 9 | Probability: 0.675 AA1: 68 AA2: 69 |
| 249, 250 | Glycosidase | 5 | Probability: 1.000 AA1: 22 AA2: 23 |
| 25, 26 | Glycosidase; ORF 012 - | 6 | 1-29 |
| | family 6 (cellulase) | | |
| 251, 252 | Glycosidase | 45 | Probability: 1.000 AA1: 21 AA2: 22 |
| 253, 254 | Glycosidase | 48 | Probability: 0.993 AA1: 22 AA2: 23 |
| 255, 256 | Glycosidase | 48 | Probability: 0.993 AA1: 22 AA2: 23 |
| 257, 258 | Glycosidase | 48 | Probability: 0.993 AA1: 22 AA2: 23 |
| 259, 260 | Glycosidase | 48 | Probability: 0.993 AA1: 22 AA2: 23 |
| 261, 262 | Glycosidase | 5 | Probability: 0.999 AA1: 24 AA2: 25 |
| 263, 264 | Glycosidase | 48 | Probability: 0.993 AA1: 22 AA2: 23 |
| 265, 266 | Glycosidase | | Probability: 0.953 AA1: 20 AA2: 21 |
| 267, 268 | Glycosidase | 6 | Probability: 1.000 AA1: 19 AA2: 20 |
| 269, 270 | Glycosidase | 5 | |
| 27, 28 | Glycosidase | 7 | |
| 271, 272 | Glycosidase | 5 | Probability: 0.994 AA1: 18 AA2: 19 |
| 273, 274 | Glycosidase | 5 | Probability: 1.000 AA1: 23 AA2: 24 |
| 275, 276 | Glycosidase | 5 | Probability: 0.997 AA1: 19 AA2: 20 |
| 277, 278 | Glycosidase | 5 | |
| 279, 280 | Glycosidase | 5 | Probability: 1.000 AA1: 22 AA2: 23 |
| 281, 282 | Glycosidase | 6 | Probability: 0.999 AA1: 18 AA2: 19 |
| 283, 284 | Glycosidase | 5 | |
| 285, 286 | Glycosidase | 5 | Probability: 1.000 AA1: 24 AA2: 25 |
| 287, 288 | Glycosidase | 5 | |
| 289, 290 | Glycosidase | 5 | Probability: 0.976 AA1: 18 AA2: 19 |
| 29, 30 | Glycosidase | 7 | Probability: 0.981 AA1: 25 AA2: 26 |
| 291, 292 | Glycosidase | 9 | |
| 293, 294 | Glycosidase | 9 | |
| 295, 296 | Glycosidase | 9 | |
| 297, 298 | Glycosidase | 9 | Probability: 0.907 AA1: 23 AA2: 24 |
| 299, 300 | Glycosidase | 9 | Probability: 0.627 AA1: 31 AA2: 32 |
| 3, 4 | Glycosidase | 6 | Probability: 1.000 AA1: 29 AA2: 30 |
| 301, 302 | Glycosidase | 9 | Probability: 0.752 AA1: 30 AA2: 31 |
| 303, 304 | Glycosidase | 9 | Probability: 0.991 AA1: 24 AA2: 25 |
| 305, 306 | Glycosidase | 5 | Probability: 0.986 AA1: 22 AA2: 23 |
| 307, 308 | Glycosidase | 5 | Probability: 0.985 AA1: 22 AA2: 23 |
| 309, 310 | Glycosidase | 9 | |
| 31, 32 | Glycosidase | 7 | Probability: 0.999 AA1: 21 AA2: 22 |
| 311, 312 | Glycosidase | 5 | Probability: 0.986 AA1: 22 AA2: 23 |
| 313, 314 | Glycosidase | 45 | Probability: 0.788 AA1: 16 AA2: 17 |
| 315, 316 | Glycosidase | 6 | Probability: 1.000 AA1: 30 AA2: 31 |
| 317, 318 | Glycosidase | 6 | Probability: 1.000 AA1: 25 AA2: 26 |
| 319, 320 | Glycosidase | 6 | Probability: 1.000 AA1: 25 AA2: 26 |
| 321, 322 | Glycosidase | 6 | Probability: 1.000 AA1: 25 AA2: 26 |
| 323, 324 | Glycosidase | 6 | Probability: 0.984 AA1: 27 AA2: 28 |
| 325, 326 | Glycosidase | 6 | Probability: 0.984 AA1: 27 AA2: 28 |
| 327, 328 | Glycosidase | 6 | Probability: 0.984 AA1: 27 AA2: 28 |
| 329, 330 | Glycosidase | 6 | Probability: 0.984 AA1: 27 AA2: 28 |
| 33, 34 | Glycosidase; | 7 | Probability: 0.994 AA1: 20 AA2: 21 |
| | Cellobiohydrolase | | |
| 331, 332 | Glycosidase | 6 | Probability: 0.984 AA1: 27 AA2: 28 |
| 333, 334 | Glycosidase | 6 | Probability: 1.000 AA1: 25 AA2: 26 |
| 335, 336 | Glycosidase | 6 | Probability: 1.000 AA1: 25 AA2: 26 |
| 337, 338 | Glycosidase | 9 | |
| 339, 340 | Glycosidase | 9 | Probability: 0.992 AA1: 21 AA2: 22 |
| 341, 342 | Glycosidase | 6 | Probability: 0.993 AA1: 19 AA2: 20 |
| 343, 344 | Glycosidase | 6 | Probability: 0.993 AA1: 19 AA2: 20 |
| 345, 346 | Glycosidase | 6 | |
| 347, 348 | Glycosidase | 45 | Probability: 0.939 AA1: 23 AA2: 24 |
| 349, 350 | Glycosidase | 6 | Probability: 1.000 AA1: 42 AA2: 43 |
| 35, 36 | Endoglucanase | 6 | Probability: 1.000 AA1: 19 AA2: 20 |
| 351, 352 | Glycosidase | 6 | Probability: 1.000 AA1: 28 AA2: 29 |
| 353, 354 | Glycosidase | 6 | |
| 355, 356 | Glycosidase | 7 | Probability: 0.999 AA1: 17 AA2: 18 |
| 357, 358 | Glycosidase | 6 | Probability: 0.964 AA1: 16 AA2: 17 |
| 359, 360 | Glycosidase; | 7 | Probability: 0.995 AA1: 23 AA2: 24 |
| | Cellobiohydrolase | | |
| 361, 362 | Glycosidase | 9 | Probability: 1.000 AA1: 27 AA2: 28 |
| 363, 364 | Glycosidase | 8 | Probability: 1.000 AA1: 25 AA2: 26 |
| 365, 366 | Glycosidase | 8 | Probability: 0.996 AA1: 21 AA2: 22 |
| 367, 368 | Glycosidase | 9 | Probability: 1.000 AA1: 32 AA2: 33 |
| 369-371 | | 7 | |
| 37, 38 | Glycosidase | 48 | Probability: 1.000 AA1: 27 AA2: 28 |
| 372-374 | | 6 | |
| 375-377 | | 6 | |
| 378-380 | | 6 | |
| 381-383 | | 6 | |
| 384-386 | | 6 | |
| 387-389 | | 6 | |
| 39, 40 | Glycosidase | 48 | |
| 390-392 | | 6 | |
| 393-395 | | 6 | |
| 396-398 | | 6 | |
| 399-401 | | 6 | |
| 402-404 | | 6 | |
| 405-407 | | 6 | |
| 408-410 | | 6 | |
| 41, 42 | Glycosidase | 5 | Probability: 1.000 AA1: 21 AA2: 22 |
| 411-413 | | 6 | |
| 414-416 | | 6 | |
| 417-419 | | 6 | |
| 420-422 | | 6 | |
| 423, 424 | β-glucosidase | | |
| 425, 426 | | | |
| 427, 428 | Alkaline | | 1-30 |
| | endoglucanase/cellulase | | |
| 429, 430 | | | Probability: 1.000 AA1: 29 AA2: 30 |
| 43, 44 | Glycosidase | 9 | Probability: 0.972 AA1: 65 AA2: 66 |
| 431, 432 | Glycosidase | | Probability: 0.998 AA1: 21 AA2: 22 |
| 433, 434 | Glycosidase | | |
| 435, 436 | Glycosidase | | |
| 437, 438 | Glycosidase | | |
| 439, 440 | Glycosidase | | |
| 441, 442 | Glycosidase | | Probability: 1.000 AA1: 26 AA2: 27 |
| 443, 444 | Glycosidase | | |
| 445, 446 | Glycosidase | | Probability: 1.000 AA1: 23 AA2: 24 |
| 447, 448 | Glycosidase | | |
| 449, 450 | | | Probability: 0.987 AA1: 28 AA2: 29 |
| 45, 46 | Glycosidase | | |
| 451, 452 | Esterase | | Probability: 1.000 AA1: 26 AA2: 27 |
| 453, 454 | Glycosidase | | |
| 455, 456 | Binding | | |
| 457, 458 | Binding | | Probability: 1.000 AA1: 19 AA2: 20 |
| 459, 460 | | | Probability: 1.000 AA1: 30 AA2: 31 |
| 461, 462 | Glycosidase | | |
| 463, 464 | | | Probability: 0.999 AA1: 24 AA2: 25 |
| 465, 466 | | | Probability: 1.000 AA1: 29 AA2: 30 |
| 467, 468 | | | Probability: 1.000 AA1: 34 AA2: 35 |
| 469, 470 | | | Probability: 0.985 AA1: 39 AA2: 40 |
| 47, 48 | Glycosidase | 9 | Probability: 1.000 AA1: 32 AA2: 33 |
| 471, 472 | | | Probability: 0.999 AA1: 22 AA2: 23 |
| 489, 490 | Endoglucanase | | |
| 49, 50 | Glycosidase | 5 | Probability: 1.000 AA1: 25 AA2: 26 |
| 491, 492 | Endoglucanase | | Probability: 1.000 AA1: 18 AA2: 19 |
| 493, 494, 707 | Endoglucanase | | Probability: 1.000 AA1: 18 AA2: 19 |
| 495, 496, 710 | Endoglucanase | | Probability: 1.000 AA1: 18 AA2: 19 |
| 497, 498, 711 | Endoglucanase | | Probability: 0.990 AA1: 15 AA2: 16 |
| 499, 500, 712 | Endoglucanase | | Probability: 1.000 AA1: 19 AA2: 20 |
| 5, 6 | Glycosidase | 6 | Probability: 1.000 AA1: 42 AA2: 43 |
| 501, 502, 713 | Endoglucanase | | Probability: 1.000 AA1: 16 AA2: 17 |
| 503, 504, 714 | Endoglucanase | | Probability: 0.999 AA1: 19 AA2: 20 |
| 505, 506, 715 | Endoglucanase | | Probability: 0.996 AA1: 16 AA2: 17 |
| 507, 508, 716 | Endoglucanase | | Probability: 1.000 AA1: 18 AA2: 19 |
| 509, 510, 717 | Endoglucanase | | Probability: 0.995 AA1: 16 AA2: 17 |
| 51, 52 | Glycosidase | 9 | Probability: 1.000 AA1: 20 AA2: 21 |
| 511, 512, 708 | Endoglucanase | | Probability: 0.977 AA1: 19 AA2: 20 |
| 513, 514, 709 | Endoglucanase | | Probability: 1.000 AA1: 19 AA2: 20 |
| 515, 516 | Glycosidase | | Probability: 1.000 AA1: 25 AA2: 26 |
| 517, 518 | Endoglucanase | | Probability: 0.977 AA1: 19 AA2: 20 |
| 519, 520 | Endoglucanase | | Probability: 0.999 AA1: 16 AA2: 17 |
| 521, 522 | Glycosidase | | Probability: 0.994 AA1: 18 AA2: 19 |
| 523, 524 | Cellobiohydrolase | | Probability: 0.965 AA1: 16 AA2: 17 |
| 525, 526 | β-glucosidase | | Probability: 0.989 AA1: 27 AA2: 28 |
| 527, 528 | B-glucosidase | | |
| 529, 530 | B-glucosidase | | |
| 53, 54 | Glycosidase | 5 | Probability: 0.995 AA1: 23 AA2: 24 |
| 531, 532 | B-glucosidase | | |
| 533, 534 | B-glucosidase | | |
| 535, 536 | B-glucosidase | | |
| 537, 538 | β-glucosidase | | |
| 539, 540 | B-glucosidase | | |
| 541, 542 | B-glucosidase | | |
| 543, 544 | B-glucosidase | | |
| 545, 546 | B-glucosidase | | |
| 547, 548 | B-glucosidase | | |
| 549, 550 | ORF 012 - family 1 (β- | | |
| | glucosidase) | | |
| 55, 56 | Glycosidase | 5 | Probability: 0.976 AA1: 34 AA2: 35 |
| 551, 552 | β-glucosidase | | |
| 553, 554 | B-glucosidase | | |
| 555, 556 | β-glucosidase | | |
| 557, 558 | B-glucosidase | | |
| 559, 560 | β-glucosidase | | |
| 561, 562 | B-glucosidase | | |
| 563, 564 | B-glucosidase | | |
| 565, 566 | B-glucosidase | | |
| 567, 568 | β-glucosidase | | |
| 569, 570 | B-glucosidase | | |
| 57, 58 | Glycosidase | 9 | Probability: 1.000 AA1: 32 AA2: 33 |
| 571, 572 | β-glucosidase | | |
| 573, 574 | β-glucosidase | | |
| 575, 576 | B-glucosidase | | |
| 577, 578 | β-glucosidase | | |
| 579, 580 | B-glucosidase | | |
| 581, 582 | β-glucosidase | | |
| 583, 584 | B-glucosidase | | Probability: 1.000 AA1: 33 AA2: 34 |
| 585, 586 | B-glucosidase | | Probability: 1.000 AA1: 22 AA2: 23 |
| 587, 588 | B-glucosidase | | Probability: 1.000 AA1: 26 AA2: 27 |
| 589, 590 | β-glucosidase | | Probability: 1.000 AA1: 25 AA2: 26 |
| 59, 60 | Glycosidase | 45 | Probability: 1.000 AA1: 21 AA2: 22 |
| 591, 592 | β-glucosidase | | Probability: 1.000 AA1: 23 AA2: 24 |
| 593, 594 | β-glucosidase | | Probability: 0.986 AA1: 29 AA2: 30 |
| 595, 596 | β-glucosidase | | Probability: 1.000 AA1: 27 AA2: 28 |
| 597, 598 | Glycosidase | | Probability: 0.950 AA1: 16 AA2: 17 |
| 599, 600 | 0 | | Probability: 0.997 AA1: 20 AA2: 21 |
| 601, 602 | Glycosidase | | Probability: 0.994 AA1: 18 AA2: 19 |
| 603, 604 | Cellobiohydrolase | | Probability: 0.965 AA1: 16 AA2: 17 |
| 605, 606 | Cellobiohydrolase | | Probability: 1.000 AA1: 27 AA2: 28 |
| 607, 608 | Cellobiohydrolase | | Probability: 0.997 AA1: 17 AA2: 18 |
| 609, 610 | Glycosidase | | Probability: 0.998 AA1: 17 AA2: 18 |
| 61, 62 | Glycosidase | 9 | Probability: 1.000 AA1: 30 AA2: 31 |
| 611, 612 | Glycosidase | | Probability: 1.000 AA1: 28 AA2: 29 |
| 613, 614 | Glycosidase | | |
| 615, 616 | Glycosidase | | Probability: 0.952 AA1: 17 AA2: 18 |
| 617, 618 | Cellobiohydrolase | | Probability: 1.000 AA1: 18 AA2: 19 |
| 619, 620 | Cellobiohydrolase | | Probability: 1.000 AA1: 18 AA2: 19 |
| 621, 622 | Xylosidase | | Probability: 1.000 AA1: 34 AA2: 35 |
| 623, 624 | Ferulic acid esterase | | Probability: 1.000 AA1: 18 AA2: 19 |
| | (FAE) | | |
| 625, 626 | Xylosidase | | Probability: 0.998 AA1: 25 AA2: 26 |
| 627, 628 | xylanase | | |
| 629, 630 | xylanase | | |
| 63, 64 | Glycosidase | 9 | Probability: 1.000 AA1: 32 AA2: 33 |
| 631, 632 | Oligomerase/Xylosidase | | |
| 633, 634 | B-glucosidase | | |
| 635, 636 | Xylosidase | | |
| 637, 638 | Endoglucanase | | Probability: 0.996 AA1: 19 AA2: 20 |
| 639, 640 | Ferulic acid esterase | | Probability: 0.997 AA1: 27 AA2: 28 |
| | (FAE) | | |
| 641, 642 | Ferulic acid esterase | | Probability: 1.000 AA1: 41 AA2: 42 |
| | (FAE) | | |
| 643, 644 | Ferulic acid esterase | | Probability: 0.997 AA1: 23 AA2: 24 |
| | (FAE) | | |
| 645, 646 | β-glucosidase/Xylosidase | | |
| 647, 648 | a-glucuronidase | | Probability: 1.000 AA1: 21 AA2: 22 |
| 649, 650 | Acetyl xylan esterase | | |
| 65, 66 | Glycosidase | 5 | Probability: 1.000 AA1: 29 AA2: 30 |
| 651, 652 | a-glucuronidase | | Probability: 0.993 AA1: 17 AA2: 18 |
| 653, 654 | a-glucuronidase | | Probability: 0.972 AA1: 25 AA2: 26 |
| 655, 656 | Xylosidase | | |
| 657, 658 | Ferulic acid esterase | | Probability: 0.975 AA1: 28 AA2: 29 |
| | (FAE) | | |
| 659, 660 | arabinofuranosidase | | |
| 661, 662 | arabinofuranosidase | | |
| 663, 664 | xylanase | | |
| 665, 666 | Endoglucanase | | |
| 667, 668 | a-glucuronidase | | |
| 669, 670 | Xylosidase | | |
| 67, 68 | Glycosidase | 5 | Probability: 1.000 AA1: 32 AA2: 33 |
| 671, 672 | Xylosidase | | |
| 673, 674 | arabinofuranosidase | | |
| 675, 676 | arabinofuranosidase | | |
| 677, 678 | arabinofuranosidase | | Probability: 1.000 AA1: 24 AA2: 25 |
| 679, 680 | a-glucuronidase | | |
| 681, 682 | arabinofuranosidase | | |
| 683, 684 | arabinofuranosidase | | Probability: 1.000 AA1: 22 AA2: 23 |
| 685, 686 | arabinofuranosidase | | Probability: 0.999 AA1: 28 AA2: 29 |
| 687, 688 | Ferulic acid esterase | | Probability: 1.000 AA1: 26 AA2: 27 |
| | (FAE) | | |
| 689, 690 | Endoglucanase | | Probability: 1.000 AA1: 19 AA2: 20 |
| 69, 70 | Glycosidase | 5 | |
| 691, 692 | Glycosidase | | Probability: 1.000 AA1: 46 AA2: 47 |
| 693, 694 | B-glucosidase | | |
| 695, 696 | Xylosidase | | |
| 697, 698 | Xylosidase | | |
| 699, 700 | Xylosidase | | |
| 7, 8 | Glycosidase | 5 | Probability: 0.993 AA1: 19 AA2: 20 |
| 71, 72 | Glycosidase | 45 | Probability: 1.000 AA1: 21 AA2: 22 |
| 718, 719 | xylanase | | Probability: 1.000 AA1: 20 AA2: 21 |
| 720, 721 | Xylosidase | | |
| 73, 74 | Glycosidase | 5 | |
| 75, 76 | Glycosidase | 9 | Probability: 1.000 AA1: 32 AA2: 33 |
| 77, 78 | Glycosidase | 5 | Probability: 0.983 AA1: 25 AA2: 26 |
| 79, 80 | Glycosidase | 5 | Probability: 1.000 AA1: 23 AA2: 24 |
| 81, 82 | Glycosidase | | |
| 83, 84 | Glycosidase | 5 | Probability: 1.000 AA1: 28 AA2: 29 |
| 85, 86 | Glycosidase | 9 | Probability: 1.000 AA1: 32 AA2: 33 |
| 87, 88 | Glycosidase | 5 | |
| 89, 90 | Glycosidase; | 5 | Probability: 0.999 AA1: 38 AA2: 39 |
| | Endoglucanase | | |
| 9, 10 | Glycosidase | 5 | Probability: 0.999 AA1: 29 AA2: 30 |
| 91, 92 | Glycosidase | 48 | Probability: 1.000 AA1: 33 AA2: 34 |
| 93, 94 | Glycosidase | 48 | Probability: 1.000 AA1: 36 AA2: 37 |
| 95, 96 | Glycosidase | 48 | Probability: 1.000 AA1: 40 AA2: 41 |
| 97, 98 | Glycosidase | 48 | Probability: 0.995 AA1: 31 AA2: 32 |
| 99, 100 | Glycosidase | 48 | Probability: 0.889 AA1: 19 AA2: 20 |

| | | Predicted EC |
|---|---|---|
| SEQ ID NO: | Predicted Signal Sequence | Number |
| 473 | | |
| 474 | | |
| 475 | | |
| 476 | | |
| 477 | | |
| 478 | | |
| 479 | | |
| 480 | | |
| 481 | | |
| 482 | | |
| 483 | | |
| 484 | | |
| 485 | | |
| 486 | | |
| 487 | | |
| 488 | | |
| 701 | | |
| 702 | | |
| 703 | | |
| 704 | | |
| 705 | | |
| 706 | | |
| 1, 2 | MSRNIRKSSFIFSLLTIIVLIASMFLQTQT | 3.2.1.91 |
| | AQA | |
| 101, 102 | MKSVLFILLVGCVLQHIHA | |
| 103, 104 | MAKRFSLIGIGLVLALGLAGGVWA | 3.2.1.4 |
| 105, 106 | | 3.2.1.4 |
| 107, 108 | MKRMSFAVSLFTFLFAVSAYS | |
| 109, 110 | | 3.2.1.4 |
| 11, 12 | MGTSLMIKSTLTGMITAVAAAVFTTSAA | 3.2.1.91 |
| | FA | |
| 111, 112 | MTAFDNAISAAKSALASA | 3.2.1.4 |
| 113, 114 | | 3.2.1.4 |
| 115, 116 | | 3.2.1.4 |
| 117, 118 | | 3.2.1.4 |
| 119, 120 | | 3.2.1.4 |
| 121, 122 | | 3.2.1.4 |
| 123, 124 | | 3.2.1.21 |
| 125, 126 | | 3.2.1.4 |
| 127, 128 | | 3.2.1.4 |
| 129, 130 | | 3.2.1.4 |
| 13, 14 | MMRTLVTSAFACLLLPLGTGQADA | |
| 131, 132 | MKKFLLCLFLPVLLAVSCPSSPA | 3.2.1.4 |
| 133, 134 | | 3.2.1.4 |
| 135, 136 | MLKMKKFKKIGIAFLAISILLTSMLSTVS | |
| | VSA | |
| 137, 138 | MAPRRRRRAVRRLLTAVTAALALPLTM | 3.2.1.8 |
| | LANGTTPAQA | |
| 139, 140 | MHPPPRRRGGVRRLLAVAVTALALPLT | |
| | MLSTGTTPARA | |
| 141, 142 | | 3.2.1.4 |
| 143, 144 | MSRKFLLFLCTLCFAVTVWPAVSCA | 3.2.1.4 |
| 145, 146 | MQRTPVIRRTRRLPAAIVLSALATFTLS | |
| | AHA | |
| 147, 148 | MKKERNFLWAGYSRRLYAMALIFVIGF | 3.2.1.4 |
| | AAAA | |
| 149, 150 | MKKIPVFLLAFLVFFAVTGCSG | 3.2.1.4 |
| 15, 16 | | 3.2.1.91 |
| 151, 152 | MQRTPVIRRIRRLPAAAIVLSALATFTIS | |
| | AHA | |
| 153, 154 | | 3.2.1.4 |
| 155, 156 | MWRYKQGGTLQRTPVIRRTRRLSAAAI | 3.2.1.4 |
| | VLSALATFAPSARA | |
| 157, 158 | | 3.2.1.4 |
| 159, 160 | MIFKKTLFFTFTFYALLLTACRSSNGGA | 3.2.1.4 |
| 161, 162 | MKKMLFAVTLFTVLSAVSVYA | 3.2.1.3 |
| 163, 164 | MSRTRTALLAAMALVAGATGSAIA | 3.2.1.91 |
| 165, 166 | MSRTRTSILAAMALVAGATGTALTAAP | 3.2.1.91 |
| | ASA | |
| 167, 168 | MTAFENAISAAKSALASA | 3.2.1.4 |
| 169, 170 | MLHKKLLECGNYHHRPIRKGRRFLKTA | 3.2.1.4 |
| | VATAAALGMLAASFMPGNYSGTSQA | |
| 17, 18 | MPRLRARTRPRRQLTALAAALSLPLGL | |
| | TAVGATTAQA | |
| 171, 172 | MRKGIKKLGSVAIAAAMTVSLISTSVYA | |
| 173, 174 | | 3.2.1.4 |
| 175, 176 | | 3.2.1.4 |
| 177, 178 | MPTQSDSKEVSVNRKRILRTASLALVM | 3.2.1.4 |
| | LALLAGGVLG | |
| 179, 180 | MLQQFNSSRWRSSVRRLSGYLTVLAA | 3.2.1.3 |
| | LLLTLVAPSARA | |
| 181, 182 | MKSNPRRETVRVRLRRGITAFAHSVVS | 3.2.1.91 |
| | PRRTHSRPATSRRSTRTLAAAAAGVLA | |
| | SALVLVGAGAAPASA | |
| 183, 184 | MNSKGAVMKFHNGLKRPATRALVAAA | 3.2.1.91 |
| | TALATMTGMVVASAGTASA | |
| 185, 186 | MGLRSASGGSKIRLRRGVVAATTAFA | 3.2.1.91 |
| | MCVMLAGVVVNQASA | |
| 187, 188 | | |
| 189, 190 | MLQQFNSSRWRSSVRRLSGYLTVLAA | 3.2.1.3 |
| | LLLTLAAPSARA | |
| 19, 20 | MNNPRILTYLLIGIVVAVLIVFA | 3.2.1.91 |
| 191, 192 | | 3.2.1.91 |
| 193, 194 | | 3.2.1.4 |
| 195, 196 | MDPGRKRITARRALTATATALALPLSM | 3.2.1.8 |
| | LATSATTARA | |
| 197, 198 | MKLVALVTAAALAGPFYA | |
| 199, 200 | MKLVALATAAALAGPFYA | |
| 201, 202 | MIVRMLALTGSVAAVGCSG | 3.2.1.91 |
| 203, 204 | MIVRMLALTGSVAAVGCSG | 3.2.1.91 |
| 205, 206 | MYSYNIANIIFYITSMKPFFTLIFMATLVNA | 3.2.1.4 |
| 207, 208 | MIKRRTVLGALPAFGLIGMQASTAAA | |
| 209, 210 | MTSHRQSARLAVFTVLLLLLMAAPAFV | 3.2.1.91 |
| | MA | |
| 21, 22 | MKKVSNARVLSFLLILVLIFGNLASVFA | 3.2.1.4 |
| 211, 212 | | 3.2.1.4 |
| 213, 214 | | 3.2.1.4 |
| 215, 216 | MSGRAMPPRPAWFAAALLAVACIIPPA | 3.2.1.91 |
| | PA | |
| 217, 218 | MTLKHASSLIRGLSLWRGALGVLAVSL | |
| | SLAACGGGAQT | |
| 219, 220 | MTLKHASSLIRGLSLWRGALGVLAVSL | |
| | SLAACGGGAQT | |
| 221, 222 | MSRTRTSLVAALALVAGTSGTVLLSAP | 3.2.1.91 |
| | AGA | |
| 223, 224 | MSRTRTSLVAALALVAGTSGTVLLSAP | 3.2.1.91 |
| | AGA | |
| 225, 226 | MLAALALLGGTSAAALVSAPAGA | 3.2.1.91 |
| 227, 228 | MSRTKTSLLAALALLGGTSAAALVSAP | 3.2.1.91 |
| | AGA | |
| 229, 230 | MSRTKTSLLAALALLGGTSAAALVSAP | 3.2.1.91 |
| | AGA | |
| 23, 24 | MKRTRYGVRSPRSAPRFGVLFGAAAA | |
| | GVLMTGA | |
| 231, 232 | MEYKFFALVAVSASVLASSAFA | |
| 233, 234 | MKKLILCLLFPMLLAFCHSASV | 3.2.1.4 |
| 235, 236 | MTLKHASSLIRGLSLWRGALGVLAVSL | |
| | SLAACGGAQT | |
| 237, 238 | MTLKHASSLIRGLSLWRGALGVLAVSL | 3.2.1.91 |
| | SLAACGGAQT | |
| 239, 240 | MTLKHASSLIRGLSLWRGALGVLAVSL | |
| | SLAACGGAQT | |
| 241, 242 | MSRHYYARGAMLLALLTMIGGLLTTQNA | 3.2.1.8 |
| 243, 244 | MRNAIFVIGGIALSVSALG | 3.2.1.91 |
| 245, 246 | MLHRTPVIRRNRRLSAAAVVLSALAAF | 3.2.1.4 |
| | TLNAHA | |
| 247, 248 | MSFFFAQIKILTLTLPPYILIGKAVTAAIH | 3.2.1.4 |
| | PPKGGTLQRTPVIRRNSRLSAAAVVLS | |
| | ALATFTIGAHA | |
| 249, 250 | MKKFFKLIGIITLAAIIGFTMA | 3.2.1.4 |
| 25, 26 | MTRRSIVRSSSNKWLVLAGAALLACTA | 3.2.1.91 |
| | LG | |
| 251, 252 | MKKMLFAFALFTVFFAVSVYA | 3.2.1.3 |
| 253, 254 | MKRLPILTILAIFVFSILPLSA | 3.2.1.4 |
| 255, 256 | MKRLPILTILAIFVFSILPLSA | 3.2.1.4 |
| 257, 258 | MKRLPILTILAIFVFSILPLSA | 3.2.1.4 |
| 259, 260 | MKRLPILTILAIFVFSILPLSA | 3.2.1.4 |
| 261, 262 | MTKRKNSKWKIVIACIVVVLLVVA | 3.2.1.4 |
| 263, 264 | MKRLPILTILAIFVFSILPLSA | 3.2.1.4 |
| 265, 266 | MRKLSLLTASLIFWAIFSIS | |
| 267, 268 | MQRISGLAAALLLANIASA | 3.2.1.91 |
| 269, 270 | | 3.2.1.4 |
| 27, 28 | | |
| 271, 272 | MKKIIFLFAAVFIFSCTS | 3.2.1.4 |
| 273, 274 | MGKIKAFAAVAALSLAVAGNLWA | 3.2.1.4 |
| 275, 276 | MKKIIILFAAAVLFSCTSS | 3.2.1.4 |
| 277, 278 | | 3.2.1.4 |
| 279, 280 | MKKIFILFAAAVLAGCSTSETA | 3.2.1.4 |
| 281, 282 | MTVYQLLFTAALAGTALA | 3.2.1.91 |
| 283, 284 | | 3.2.1.4 |
| 285, 286 | MRKKSTLSLVGAAVALVCASAAVA | 3.2.1.4 |
| 287, 288 | | 3.2.1.4 |
| 289, 290 | MKKILILFAAAVLFYCTS | 3.2.1.4 |
| 29, 30 | MSAALSYRIYKNALLFTAFLTAARA | |
| 291, 292 | | 3.2.1.4 |
| 293, 294 | | 3.2.1.4 |
| 295, 296 | | 3.2.1.4 |
| 297, 298 | MIFYILPMKPFLTLIFMATLLNA | 3.2.1.4 |
| 299, 300 | MTLSRGPPAIFYILSMKPFFALIFMVTLV | 3.2.1.4 |
| | NA | |
| 3, 4 | MRLKTLATATAAAAVVAGTAVLWPGSA | 3.2.1.91 |
| | SA | |
| 301, 302 | MILSRGPAIFYILSMKPFFALIFMVTLVNA | 3.2.1.4 |
| 303, 304 | MKHPFALIFMAIPSLFLFTQCQNA | 3.2.1.4 |
| 305, 306 | MKKYLCLIAVFLFSCTSEIESA | 3.2.1.4 |
| 307, 308 | MKKYLCLIAVSLFSCTSEIESA | 3.2.1.4 |
| 309, 310 | | 3.2.1.4 |
| 31, 32 | MSSFQIYRAALLLSILATANA | |
| 311, 312 | MKKYLCLIAVFLFSCTSEIESA | 3.2.1.4 |
| 313, 314 | MRLFLVAVALVIAVLG | |
| 315, 316 | MTRTRTAMLAALTLVAGASGTALAAHS | 3.2.1.91 |
| | ASA | |
| 317, 318 | MMLSRRFGLALSASLLLAAGCGARA | 3.2.1.91 |
| 319, 320 | MMLSRRFGLSLSASLLLAAGCGARA | 3.2.1.91 |
| 321, 322 | MMLSRRFGLALSASLLLAAGCGARA | 3.2.1.91 |
| 323, 324 | MSTLRTVVIGLLAVGLVAGGRPAPGLA | |
| 325, 326 | MSTLRTVVIGLLAVGLVAGGRPAPGLA | |
| 327, 328 | MSTLRTVVIGLLAVGLVAGGRPAPGLA | |
| 329, 330 | MSTLRTVVIGLLAVGLVAGGRPAPGLA | |
| 33, 34 | MYQKLAAISAFLAAARAQQV | |
| 331, 332 | MSTLRTVVIGLLAVGLVAGGRPAPGLA | |
| 333, 334 | MMLSRRFGLALSASLLLAAGCGARA | 3.2.1.91 |
| 335, 336 | MMLSRRFGLALSASLLLAAGCGARA | 3.2.1.91 |
| 337, 338 | | 3.2.1.4 |
| 339, 340 | MNVSYPLFTIAITGFFFSAQA | |
| 341, 342 | MRSPVVVVAVLVGSLFATS | 3.2.1.91 |
| 343, 344 | MRSPVVVVAVLVGSLFATS | 3.2.1.91 |
| 345, 346 | | 3.2.1.91 |
| 347, 348 | MKCKYMYFFFVSLLIFACNNSNN | |
| 349, 350 | MSTLKVKQVSLVLTI LAVLVATFMGFTQ | 3.2.1.91 |
| | KSARAAAICSPATA | |
| 35, 36 | MRFPSIFTAVLFAASSALA | 3.2.1.91 |
| 351, 352 | MNPLKSLISCSPGLLGLFLLGGIHVANA | 3.2.1.91 |
| 353, 354 | | 3.2.1.91 |
| 355, 356 | MYQRALLFSALMAGATA | |
| 357, 358 | MVVGILATLATLATLA | 3.2.1.91 |
| 359, 360 | MSALNSFNMYKSALILGSLLATA | |
| 361, 362 | MKKILAFLLTVALVAVVAIPQAVVSFA | |
| 363, 364 | MKKIPLLMLLSAIIFLSLHPTLSYA | 3.2.1.14 |
| 365, 366 | MLILAVLGVYMLAMPANTVSA | |
| 367, 368 | MQRTPVIRRIRRLPAAAIVLSALATFTIS | |
| | AHA | |
| 369-371 | | |
| 37, 38 | MVKSRKISILLAVAMLVSIMIPTTAFA | |
| 372-374 | | |
| 375-377 | | |
| 378-380 | | |
| 381-383 | | |
| 384-386 | | |
| 387-389 | | |
| 39, 40 | | 3.2.1.4 |
| 390-392 | | |
| 393-395 | | |
| 396-398 | | |
| 399-401 | | |
| 402-404 | | |
| 405-407 | | |
| 408-410 | | |
| 41, 42 | MKTVLRVLFLAVAIVASVANA | 3.2.1.4 |
| 411-413 | | |
| 414-416 | | |
| 417-419 | | |
| 420-422 | | |
| 423, 424 | | 3.2.1.21 |
| 425, 426 | | 3.2.1.4 |
| 427, 428 | MSCRTLMSRRVGWGLLLWGGLFLRT | 3.2.1.4 |
| | GSVTG | |
| 429, 430 | MRKIILKFCALMMVVILIVSILQILPVFA | 3.2.1.4 |
| 43, 44 | MDLALKNLTFAAPSYILMNRPQPVAIHP | 3.2.1.4 |
| | PKGGSLQRTPVIRRNSRLSAAAAVLSA | |
| | LAAFTLSAHA | |
| 431, 432 | MKGLIAAALAGLAFGASLSWG | 3.2.1.8 |
| 433, 434 | | 3.2.1.8 |
| 435, 436 | | 3.2.1. |
| 437, 438 | | 3.2.1.4 |
| 439, 440 | | 3.2.1.8 |
| 441, 442 | MARSKRVLAWIMSSVLLISMAMPSFA | 3.2.1.8 |
| 443, 444 | | 3.2.1.8 |
| 445, 446 | MLKKLALAAGIAAATLAASGSHG | |
| 447, 448 | | 3.2.1.4 |
| 449, 450 | MALRSRLVSLAAVLATLLGGLGLSFLWQ | 3.2.1.8 |
| 45, 46 | | 3.2.1.55 |
| 451, 452 | MRHGLSLSLRAGALLCVAAFSGASHA | |
| 453, 454 | | |
| 455, 456 | | |
| 457, 458 | MRSRLAAFGALAGLTATLA | |
| 459, 460 | MRKKSVGSAVVALGVAGATLLATGSA | |
| | GSHG | |
| 461, 462 | | 3.2.1. |
| 463, 464 | MSMITPKTKSYGLAAMLSLGLAVA | 3.2.1.4 |
| 465, 466 | MKRSISIFITCLLITLLTMGGMIASPASA | 3.2.1.4 |
| 467, 468 | MKKRQGFIKKGLVLGVSLLLLALIMMSA | 3.2.1.8 |
| | TSQTSA | |
| 469, 470 | MSSFKASAINPRMAGTLTRSLYAAGFS | 3.2.1.4 |
| | LAVSTLSTQAYA | |
| 47, 48 | MQRTSVIRRIRRPVAAAAFLSALAAFTL | 3.2.1.4 |
| | SVHA | |
| 471, 472 | MVRRTRLLTLAAVLATLLGSLG | 3.2.1.4 |
| 489, 490 | | 3.2.1.4 |
| 49, 50 | MKKFFICLLLPVLLAVSCPSSPVSQ | 3.2.1.4 |
| 491, 492 | MKFQSTLLLAAAAGSALA | 3.2.1.4 |
| 493, 494, 707 | MKFQSTLLLAAAAGSALA | 3.2.1.4 |
| 495, 496, 710 | MLLQNLFAAATLAAAAFA | 3.2.1.4 |
| 497, 498, 711 | MKLLTVAALTGGALA | 3.2.1.4 |
| 499, 500, 712 | MKSLFALSLFAGLSVAQNA | 3.2.1.4 |
| 5, 6 | MSGEPHVSLRLSRPRRRTAILAAVAAC | 3.2.1.8 |
| | TVTAGAWLATGTASA | |
| 501, 502, 713 | MRNLLALFALAGPALA | 3.2.1.4 |
| 503, 504, 714 | MRSALLVVAGASLALSACA | 3.2.1.4 |
| 505, 506, 715 | MKSSVLAGIFATGAAA | 3.2.1.4 |
| 507, 508, 716 | MKFLNIILGAAAAGSALA | 3.2.1.4 |
| 509, 510, 717 | MKTSVLAGIFATGAAA | 3.2.1.4 |
| 51, 52 | MNRIAFLALVACCMPWSAQS | |
| 511, 512, 708 | MKTLSLVAVLLVQAWTASS | 3.2.1.4 |
| 513, 514, 709 | MKSLFALSLFAGLSVAQNA | 3.2.1.4 |
| 515, 516 | MKKIVSLVCVLVMLVSILGSFSVVA | 3.2.1.4 |
| 517, 518 | MKTLSLVAVLLVQAWTASS | 3.2.1.4 |
| 519, 520 | MRYDLLLAASAALALA | 3.2.1.4 |
| 521, 522 | MRYTWSVAAALLPCAIQA | 3.2.1.91 |
| 523, 524 | MSLLLTALSLVAAAKA | |
| 525, 526 | MALSTVSKVMLLTCAAVLLTIPGCNSA | 3.2.1.21 |
| 527, 528 | | 3.2.1.52 |
| 529, 530 | | 3.2.1.21 |
| 53, 54 | MKKLFGLSGIITIAAIIGFSIAA | 3.2.1.4 |
| 531, 532 | | 3.2.1.21 |
| 533, 534 | | 3.2.1.21 |
| 535, 536 | | 3.2.1.21 |
| 537, 538 | | 3.2.1.21 |
| 539, 540 | | 3.2.1.21 |
| 541, 542 | | 3.2.1.21 |
| 543, 544 | | 3.2.1.21 |
| 545, 546 | | 3.2.1.21 |
| 547, 548 | | 3.2.1.21 |
| 549, 550 | | 3.2.1.21 |
| 55, 56 | MILLKKEAFMRKLFGSSGIITIAAIIGFSIA | 3.2.1.4 |
| | ACG | |
| 551, 552 | | 3.2.1.21 |
| 553, 554 | | 3.2.1.21 |
| 555, 556 | | 3.2.1.23 |
| 557, 558 | | 3.2.1.21 |
| 559, 560 | | 3.2.1.23 |
| 561, 562 | | 3.2.1.21 |
| 563, 564 | | 3.2.1.21 |
| 565, 566 | | 3.2.1.21 |
| 567, 568 | | |
| 569, 570 | | 3.2.1.21 |
| 57, 58 | MQRTSVIRRIRRPAGAASFLFALATFS | 3.2.1.4 |
| | MSARA | |
| 571, 572 | | 3.2.1.21 |
| 573, 574 | | 3.2.1.21 |
| 575, 576 | | 3.2.1.21 |
| 577, 578 | | 3.2.1.21 |
| 579, 580 | | 3.2.1.21 |
| 581, 582 | | 3.2.1.21 |
| 583, 584 | MLSNRRLIRTIPLGAAAYSVLLGLAGCS | 3.2.1.21 |
| | QSTVA | |
| 585, 586 | MKIRSLLLLISILLGVVSPGFG | 3.2.1.21 |
| 587, 588 | MNTGWRGSFLAVAAVSLAALATSSVA | 3.2.1.21 |
| 589, 590 | MTDRDVSRRALLSLAAVAAATPAVA | 3.2.1.21 |
| 59, 60 | MKKMFFAVAMLVMFFAVGAYA | |
| 591, 592 | MNRRELLASTLAFSAASALPAAA | 3.2.1.21 |
| 593, 594 | MNCTLKPMARVVAGCVATLALAACGS | 3.2.1.21 |
| | DTG | |
| 595, 596 | MSLFRPHPLKTALATVLLGALTGQALA | 3.2.1.21 |
| 597, 598 | MIVGILTTLATLATLA | 3.2.1.91 |
| 599, 600 | MYRKLAVISAFLAAARAQQV | |
| 601, 602 | MRYTWSVAAALLPCAIQA | 3.2.1.91 |
| 603, 604 | MSLLLTALSLVAAAKA | |
| 605, 606 | MKGSISYQIYKGALLLSSLLASVSAQG | |
| 607, 608 | MLTLAFLSLLAAANAQK | |
| 609, 610 | MHQRALLFSAFWTAVQA | |
| 61, 62 | MQKTPVIQPIRRPATAALVLAAALAVSA | |
| | RA | |
| 611, 612 | MLIRLAAAGALLLGAVFVAVSPAAAATA | 3.2.1.8 |
| 613, 614 | | 3.2.1.4 |
| 615, 616 | MYRVIATASALIATARA | |
| 617, 618 | MFSKTALLSSIFAAAATA | |
| 619, 620 | MQRTSAWALLLLAQIATA | 3.2.1.91 |
| 621, 622 | MHHDSNDTTSTRRRFLATVAAAGAAG | 3.2.1.21 |
| | ATSNLAFA | |
| 623, 624 | MKRLLCSLLLALSLVTYA | 3.5.2.6 |
| 625, 626 | MKKRAFSFSLCVAIISTFWLPVAHM | 3.2.1.21 |
| 627, 628 | | 3.2.1.8 |
| 629, 630 | | 3.2.1.8 |
| 63, 64 | MPKTPVIRRIRRHVAVAAFLSALAAFAA | 3.2.1.4 |
| | SARA | |
| 631, 632 | | 3.2.1.21 |
| 633, 634 | | 3.2.1.21 |
| 635, 636 | | 3.2.1.55 |
| 637, 638 | KVTRSSAAMLLLNGAVSVA | 3.2.1.4 |
| 639, 640 | MNAAQLLSAITGSVTVLALLAQAPARA | 3.1.1.73 |
| 641, 642 | MPKTSTTDPWRAIRTRAQRTVRLLAG | |
| | GSLLSLALTGAPALA | |
| 643, 644 | MHKFISMGAFSVVAIACSSLLMG | 3.1.1. |
| 645, 646 | | 3.2.1.21 |
| 647, 648 | MRLFAAFCLLLTALLATPAVA | 3.2.1.139 |
| 649, 650 | | 3.1.1.73 |
| 65, 66 | MYRYSLTFLFLLSSFFVLAMSCPSSPV | 3.2.1.4 |
| | SQ | |
| 651, 652 | MRLLFTTLLWAVGGALA | 3.2.1.139 |
| 653, 654 | MKNVQSFYLKALFAALFLFSLWLKA | 3.2.1.139 |
| 655, 656 | | |
| 657, 658 | MNHFASKSLRMAWQPGLLATTVLPLA | 3.2.1.8 |
| | AA | |
| 659, 660 | | 3.2.1.55 |
| 661, 662 | | 3.2.1.55 |
| 663, 664 | | 3.2.1.8 |
| 665, 666 | | |
| 667, 668 | | 3.2.1.3 |
| 669, 670 | | 3.2.1.21 |
| 67, 68 | MSKKHSNHVNARSFLSTAAMILIGATLF | 3.2.1.4 |
| | GANA | |
| 671, 672 | | 3.2.1.37 |
| 673, 674 | | 3.2.1.55 |
| 675, 676 | | 3.2.1.55 |
| 677, 678 | MFDRVARGALALAVTCAFVLPAEA | 3.2.1.55 |
| 679, 680 | | 3.2.1.8 |
| 681, 682 | | 3.2.1.21 |
| 683, 684 | MKSIKHIAAAAALGLAVLTASA | 3.2.1.55 |
| 685, 686 | MTSGRNTCVCLLLIVLAIGLLSKPPASA | 3.2.1.55 |
| 687, 688 | MLRPASLFALGALLFLSLLDSVSAAT | |
| 689, 690 | MRFPSIFTAVLFAASSALA | 3.2.1.91 |
| 69, 70 | | 3.2.1.4 |
| 691, 692 | MSVTEPPPRRRGRHSRARRFLTSLGA | 3.2.1.4 |
| | TAALTAGMLGVPLATGTAHA | |
| 693, 694 | | 3.2.1.21 |
| 695, 696 | | |
| 697, 698 | | |
| 699, 700 | | |
| 7, 8 | MKSVLALALIVSINLVLLA | 3.2.1.4 |
| 71, 72 | MKKMFFAVALCVVFLAVGAHA | |
| 718, 719 | MKRPLVNLLTTACLLVAANA | 3.2.1.8 |
| 720, 721 | | |
| 73, 74 | | 3.2.1.4 |
| 75, 76 | MQRTPVIRRTRRLSAAAIVLSALAAFAP | 3.2.1.4 |
| | SARA | |
| 77, 78 | MKKVILILPLVILFALMDCTSSVNK | 3.2.1.4 |
| 79, 80 | MKKFLLCLLVPVLLAVSCPSSPA | 3.2.1.4 |
| 81, 82 | | 3.2.1.55 |
| 83, 84 | MNFRKKLLFTFIIYTLLLTFCRSSNGEA | 3.2.1.4 |
| 85, 86 | MQRTPVIRRTRRLSAAAIVLSALAAFAP | 3.2.1.4 |
| | SARA | |
| 87, 88 | | 3.2.1.4 |
| 89, 90 | MFFVKDFCKGEGNVKKIVSLVCVLVML | 3.2.1.4 |
| | VSILGSFSVVA | |
| 9, 10 | MREIILKSGALLMVVILIVSILQILTVFA | 3.2.1.4 |
| 91, 92 | MKGEEERMVKRKISVLLAAAMLVSALT | |
| | PMTAFA | |
| 93, 94 | MRLKKLKNAVVATGLALGMLSTTALSA | 3.2.1.4 |
| | LNFTTTSLA | |
| 95, 96 | MPKMMKLSLIKKPISIMMATVLFLSLTT | 3.2.1.4 |
| | GLFNFRPQTAHA | |
| 97, 98 | MILNRWRPRSACAMKWGSLIVAAFVST | |
| | GAIG | |
| 99, 100 | MKSVLFILLVGCVLQHIHA | |

TABLE 3

| SEQ ID NO: | NR Description | NR Accession Code | NR Evalue | NR Organism | Geneseq Protein Description |
|---|---|---|---|---|---|
| 1, 2 | glycoside hydrolase, family 6 [Herpetosiphon | 113938252 | 1.00E−106 | Herpetosiphon | Vibrio harveyi endoglucanase DNA. |
| | aurantiacus ATCC 23779] | | | aurantiacus ATCC | |
| | gi\|113900042\|gb\|EAU19035.1\|glycoside hydrolase, family 6 | | | 23779 | |
| | [Herpetosiphon aurantiacus ATCC 23779] | | | | |
| 3, 4 | Endoglucanase A precursor (endo-1,4-beta-glucanase) | 121805 | 1.00E−139 | Thermobispora | Amino acid sequence of a gene down- |
| | (cellulase). | | | bispora | regulated during carbon starvation. |
| 5, 6 | endo-beta-1,4-glucanase; McenA [Micromonospora | 1009722 | 1.00E−169 | Micromonospora | M. xanthus protein seq., seq id 9726. |
| | cellulolyticum]. | | | cellulolyticum | |
| 7, 8 | cellulase (EC 3.2.1.4), alkaline - Bacillus sp. (strain KSM-S237). | 25336830 | 0 | Bacillus sp. | Bacillus alkaline cellulase enzyme |
| | | | | | amino acid sequence - SEQ ID 4. |
| 9, | endoglucanase [Anaerocellum thermophilum]. | 1483210 | 0 | Anaerocellum | Bacillus sp alkaline cellulase PCR primer SEQ ID 22. |
| 10 | | | | thermophilum | |
| 11, | Cellobiohydrolase A (1 4-beta-cellobiosidase A)-like | 90021917 | 0 | Saccharophagus | Vibrio harveyi endoglucanase DNA. |
| 12 | [Saccharophagus degradans 2-40] | | | degradans 2-40 | |
| 13, | Endoglucanase 1 precursor (endo-1,4-beta-glucanase 1) | 544459 | 1.00E−129 | Streptomyces halstedii | A. gossypii/S. halstedii fusion construct |
| 14 | (cellulase 1) (CMCASE I) (CEL1). | | | | containing cellulase DNA. |
| 15, | secreted cellulase [Streptomyces coelicolor A3(2)] | 21224850 | 0 | Streptomyces | Exo-cellobiohydrolase cbh1 catalytic |
| 16 | | | | coelicolor A3(2) | domain. |
| 17, | cellulose 1;4-beta-cellobiosidase [Streptomyces | 29828397 | 0 | Streptomyces | Bacterial polypeptide #10001. |
| 18 | avermitilis MA-4680] | | | avermitilis MA-4680 | |
| 19, | Cellulase [Acidothermus cellulolyticus 11B] | 88932594 | 1.00E−106 | Acidothermus | Saccharothrix australiensis endo-beta-1,4-glucanase |
| 20 | gi\|88911374\|gb\|EAR30819.1\|Cellulase [Acidothermus | | | cellulolyticus 11B | gene. |
| | cellulolyticus 11B] | | | | |
| 21, | Endoglucanase precursor (endo-1,4-beta-glucanase) | 121838 | 0 | Bacillus sp. KSM-635 | Full length Bacillus sp. alkaline |
| 22 | (alkaline cellulase) | | | | cellulase. |
| 23, | endoglucanase A precursor (Endo-1;4-beta-glucanase) | 111224344 | 3.00E−78 | Frankia alni ACN14a | Amino acid sequence of a gene down- |
| 24 | (Cellulase) [Frankia alni ACN14a] | | | | regulated during carbon starvation. |
| 25, | Cellulase [Mycobacterium sp. JLS] | 92909181 | 2.00E−69 | Mycobacterium sp. | Amino acid sequence of a gene down- |
| 26 | gi\|92913044\|ref\|ZP_01281673.1\|Cellulase | | | JLS | regulated during carbon starvation. |
| | [Mycobacterium sp. KMS] | | | | |
| | gi\|108802261\|ref\|YP_642458.1\|Cellulase | | | | |
| | [Mycobacterium sp. MCS] gi\|92433643\|gb\|EAS92976.1\| | | | | |
| | Cellulase [Mycobacterium sp. JLS] | | | | |
| | gi\|92442306\|gb\|EAT00144.1\|Cell | | | | |
| 27, | exo-cellobiohydrolase [Penicillium chrysogenum] | 55775695 | 1.00E−74 | Penicillium | Cellobiohydrolase CBH protein |
| 28 | | | | chrysogenum | fragment. |
| 29, | exo-cellobiohydrolase [Penicillium chrysogenum] | 55775695 | 0 | Penicillium | Cellobiohydrolase I activity protein SEQ |
| 30 | | | | chrysogenum | ID No 16. |
| 31, | 1,4-beta-D-glucan cellobiohydrolase B precursor | 6164684 | 0 | Aspergillus niger | Cellobiohydrolase CBH protein |
| 32 | [Aspergillus niger]. | | | | fragment. |
| 33, | Exoglucanase I precursor (Exocellobiohydrolase I) | 50400675 | 0 | | PCR primer Mcbh1-N of the |
| 34 | (CBHI) (1;4-beta-cellobiohydrolase) | | | | specification. |
| 35, | hypothetical protein SNOG_05090 [Phaeosphaeria | 111066361 | 1.00E−170 | Phaeosphaeria | PCR primer for H. insolens Cel6B |
| 36 | nodorum SN15] | | | nodorum SN15 | fungal cellulase coding sequence. |
| 37, | Glycoside hydrolase, family 48: Clostridium cellulosome | 67875068 | 0 | Clostridium | Clostridium josui cellulose degrading cellulase D |
| 38 | enzyme, dockerin type I [Clostridium thermocellum | | | thermocellum ATCC | protein. |
| | ATCC 27405] gi\|729647\|sp\|P38686\|GUNS_CLOTM | | | 27405 | |
| | Endoglucanase SS precursor (EGSS) (Endo-1,4-beta- | | | | |
| | glucanase) (Cellulase SS) gi\|289859\|gb\|AAA23226.1\| | | | | |
| | cellula | | | | |
| 39, | EXOGLUCANASE II PRECURSOR | 1708082 | 0 | Clostridium | Clostridium josui cellulose degrading |
| 40 | (EXOCELLOBIOHYDROLASE II) (1,4-BETA- | | | stercorarium | cellulase D protein. |
| | CELLOBIOHYDROLASE II) (AVICELASE II). | | | | |
| 41, | endoglucanase. | 228944 | 5.00E−59 | Prevotella ruminicola | Cow cellulase DNA clones pBKRR 2 |
| 42 | | | | | and pBKRR 16 SEQ ID NO: 3. |
| 43, | cellulase [uncultured bacterium] | 56675038 | 1.00E−118 | uncultured bacterium | X campestris umce19A cellulase gene |
| 44 | | | | | SeqID1. |
| 45, | cellulose-binding protein [Fibrobacter succinogenes]. | 1620001 | 0 | Fibrobacter | Alicyclobacillus sp. DSM 15716 |
| 46 | | | | succinogenes | functional polypeptide coding |
| | | | | | sequence. |
| 47, | endo-1;4-beta-D-glucanase [uncultured bacterium] | 78926855 | 1.00E−117 | uncultured bacterium | X campestris umce19A cellulase gene |
| 48 | | | | | SeqID1. |
| 49, | endoglucanase. | 228944 | 2.00E−71 | Prevotella ruminicola | Cow cellulase DNA clones pBKRR 2 |
| 50 | | | | | and pBKRR 16 SEQ ID NO: 3. |
| 51, | cellulase [Xanthomonas campestris pv. campestris str. | 21231824 | 0 | Xanthomonas | X campestris umce19A cellulase gene |
| 52 | ATCC 33913]. | | | campestris pv. | SeqID1. |
| | | | | campestris str. ATCC | |
| | | | | 33913 | |
| 53, | Endoglucanase A precursor (endo-1,4-beta-glucanase A) | 1708079 | 4.00E−77 | Clostridium | Amino acid sequence of a CelE |
| 54 | (cellulase A) | | | longisporum | cellulase polypeptide. |
| 55, | Endoglucanase family 5 [Clostridium acetobutylicum]. | 15894113 | 1.00E−77 | Clostridium | Amino acid sequence of a CelE |
| 56 | | | | acetobutylicum | cellulase polypeptide. |
| 57, | endo-1;4-beta-D-glucanase [uncultured bacterium] | 78926855 | 1.00E−119 | uncultured bacterium | X campestris umce19A cellulase gene |
| 58 | | | | | SeqID1. |
| 59, | hypothetical protein SNOG_11303 [Phaeosphaeria | 111059891 | 2.00E−43 | Phaeosphaeria | Endoglucanase fusion protein SEQ ID |
| 60 | nodorum SN15] | | | nodorum SN15 | NO 2B. |
| 61, | cellulase [uncultured bacterium] | 56675038 | 1.00E−119 | uncultured bacterium | X campestris umce19A cellulase gene |
| 62 | | | | | SeqID1. |
| 63, | endo-1;4-beta-D-glucanase [uncultured bacterium] | 78926855 | 1.00E−119 | uncultured bacterium | X campestris umce19A cellulase gene |
| 64 | | | | | SeqID1. |
| 65, | endoglucanase. | 228944 | 3.00E−65 | Prevotella ruminicola | P. pabuli xyloglucanase XYG1022 DNA |
| 66 | | | | | amplifying PCR primer 189585. |
| 67, | ENDOGLUCANASE B PRECURSOR (ENDO-1,4- | 121814 | 7.00E−51 | Clostridium | P. pabuli xyloglucanase XYG1022 DNA |
| 68 | BETA-GLUCANASE B) (CELLULASE B). | | | cellulovorans | amplifying PCR primer 189585. |
| 69, | cellodextrinase [uncultured bacterium] | 91766360 | 7.00E−90 | uncultured bacterium | Anti-biofilm polypeptide #7. |
| 70 | | | | | |
| 71, | endo-beta-1,4-D-glucanase [Rhizopus oryzae]. | 27530542 | 3.00E−42 | Rhizopus oryzae | Humicola insolens endoglucanase- |
| 72 | | | | | related protein. |
| 73, | cellulase [unidentified microorganism] | 82524122 | 6.00E−73 | unidentified | Cow cellulase DNA clones pBKRR 2 |
| 74 | | | | microorganism | and pBKRR 16 SEQ ID NO: 3. |
| 75, | endo-1;4-beta-D-glucanase [uncultured bacterium] | 78926855 | 1.00E−117 | uncultured bacterium | X campestris umce19A cellulase gene |
| 76 | | | | | SeqID1. |
| 77, | endo-1;4-beta-glucanase [Streptomyces avermitilis MA- | 29828396 | 4.00E−29 | Streptomyces | Orthosomycin biosynthetic polypeptide |
| 78 | 4680] | | | avermitilis MA-4680 | SEQ ID NO 273. |
| 79, | endoglucanase. | 228944 | 8.00E−73 | Prevotella ruminicola | Cow cellulase DNA clones pBKRR 2 |
| 80 | | | | | and pBKRR 16 SEQ ID NO: 3. |
| 81, | cellulose-binding protein [Fibrobacter succinogenes]. | 1620001 | 0 | Fibrobacter | Alicyclobacillus sp. DSM 15716 |
| 82 | | | | succinogenes | functional polypeptide coding |
| | | | | | sequence. |
| 83, | cellulase [unidentified microorganism] | 82524122 | 1.00E−65 | unidentified | Cow cellulase DNA clones pBKRR 2 |
| 84 | | | | microorganism | and pBKRR 16 SEQ ID NO: 3. |
| 85, | endo-1;4-beta-D-glucanase [uncultured bacterium] | 78926855 | 1.00E−117 | uncultured bacterium | X campestris umce19A cellulase gene |
| 86 | | | | | SeqID1. |
| 87, | cellulase [unidentified microorganism] | 82524122 | 2.00E−72 | unidentified | Cow cellulase DNA clones pBKRR 2 |
| 88 | | | | microorganism | and pBKRR 16 SEQ ID NO: 3. |
| 89, | Lipolytic enzyme, G-D-S-L:Glycoside hydrolase, family | 67876012 | 0 | Clostridium | Orpinomyces cellulase CelB cDNA. |
| 90 | 5:Clostridium cellulosome enzyme, dockerin type I | | | thermocellum ATCC | |
| | [Clostridium thermocellum ATCC 27405] | | | 27405 | |
| | gi\|67850336\|gb\|EAM45917.1\|Lipolytic enzyme, G-D-S- | | | | |
| | L:Glycoside hydrolase, family 5:Clostridium cellulosome | | | | |
| | enzyme | | | | |
| 91, | Glycoside hydrolase, family 48: Clostridium cellulosome | 67875068 | 0 | Clostridium | Clostridium josui cellulose degrading |
| 92 | enzyme, dockerin type I [Clostridium thermocellum | | | thermocellum ATCC | cellulase D protein. |
| | ATCC 27405] gi\|729647\|sp\|P38686\|GUNS_CLOTM | | | 27405 | |
| | Endoglucanase SS precursor (EGSS) (Endo-1,4-beta- | | | | |
| | glucanase) (Cellulase SS) gi\|289859\|gb\|AAA23226.1\| | | | | |
| | cellula | | | | |
| 93, | EXOGLUCANASE II PRECURSOR | 1708082 | 0 | Clostridium | Clostridium josui cellulose degrading |
| 94 | (EXOCELLOBIOHYDROLASE II) (1,4-BETA- | | | stercorarium | cellulase D protein. |
| | CELLOBIOHYDROLASE II) (AVICELASE II). | | | | |
| 95, | cellulose 1,4-beta-cellobiosidase [Paenibacillus sp. BP- | 21449824 | 0 | Paenibacillus sp. BP- | Clostridium josui cellulose degrading |
| 96 | 23]. | | | 23 | cellulase D protein. |
| 97, | glycoside hydrolase, family 48 [Herpetosiphon | 113939770 | 0 | Herpetosiphon | Clostridium josui cellulose degrading |
| 98 | aurantiacus ATCC 23779] | | | aurantiacus ATCC | cellulase D protein. |
| | gi\|113898624\|gb\|EAU17637.1\|glycoside hydrolase, | | | 23779 | |
| | family 48 [Herpetosiphon aurantiacus ATCC 23779] | | | | |
| 99, | active phase-associated protein II [Gastrophysa | 95113612 | 0 | Gastrophysa | Exo-cellobiohydrolase cbh1 catalytic |
| 100 | atrocyanea] | | | atrocyanea | domain. |
| 101, | active phase-associated protein II [Gastrophysa | 95113612 | 0 | Gastrophysa | Exo-cellobiohydrolase cbh1 catalytic |
| 102 | atrocyanea] | | | atrocyanea | domain. |
| 103, | Cellulase [Saccharophagus degradans 2-40] | 90022881 | 1.00E−55 | Saccharophagus | Microbulbifer degradans cellulase |
| 104 | | | | degradans 2-40 | system protein - SEQ ID 8. |
| 105, | ENDOGLUCANASE A PRECURSOR (ENDO-1,4- | 1708079 | 5.00E−75 | Clostridium | Amino acid sequence of a CelE |
| 106 | BETA-GLUCANASE A) (CELLULASE A). | | | longisporum | cellulase polypeptide. |
| 107, | hypothetical protein SNOG_11303 [Phaeosphaeria | 111059891 | 2.00E−43 | Phaeosphaeria | Glycosyl hydrolase family 11 xylanase |
| 108 | nodorum SN15] | | | nodorum SN15 | second conserved sequence. |
| 109, | endoglucanase 3. | 666885 | 4.00E−34 | Fibrobacter intestinalis | Glucose isomerase SEQ ID NO 20. |
| 110 | | | | | |
| 111, | endoglucanase - Clostridium cellulovorans. | 98588 | 4.00E−84 | Clostridium | Amino acid sequence of a CelE |
| 112 | | | | cellulovorans | cellulase polypeptide. |
| 113, | Endoglucanase family 5 [Clostridium acetobutylicum]. | 15894113 | 2.00E−68 | Clostridium | Amino acid sequence of a CelE |
| 114 | | | | acetobutylicum | cellulase polypeptide. |
| 115, | ENDOGLUCANASE A PRECURSOR (ENDO-1,4- | 1708078 | 1.00E−116 | Caldicellulosiruptor | A. cellulolyticus Gux1 protein FN_III |
| 116 | BETA-GLUCANASE A) (CELLULASE A). | | | saccharolyticus | domain fragment. |
| 117, | cellulase/endoglucanase [unidentified microorganism] | 82524100 | 9.00E−68 | unidentified | Cow cellulase DNA clones pBKRR 2 |
| 118 | | | | microorganism | and pBKRR 16 SEQ ID NO: 3. |
| 119, | endoglucanase. | 228944 | 3.00E−70 | Prevotella ruminicola | Cow cellulase DNA clones pBKRR 2 |
| 120 | | | | | and pBKRR 16 SEQ ID NO: 3. |
| 121, | Endoglucanase B precursor (endo-1,4-beta-glucanase) | 121789 | 6.00E−92 | Bacillus sp. (strain N-4/ | P300-CelB fusion construct 4 |
| 122 | (cellulase) | | | JCM 9156) | polypeptide product. |
| 123 | Beta-glucosidase [Pseudoalteromonas atlantica T6c] | 109897152 | 1.00E−131 | Pseudoalteromonas | Vibrio harveyi endoglucanase DNA. |
| 124 | | | | atlantica T6c | |
| 125, | cellodextrinase. | 488281 | 2.00E−96 | Fibrobacter | Vibrio harveyi endoglucanase DNA. |
| 126 | | | | succinogenes | |
| 127, | cellodextrinase. | 488281 | 1.00E−96 | Fibrobacter | Vibrio harveyi endoglucanase DNA. |
| 128 | | | | succinogenes | |
| 129, | glycoside hydrolase; family 5 [Acidobacteria bacterium | 94968716 | 5.00E−37 | Acidobacteria | Glucose isomerase SEQ ID NO 20. |
| 130 | Ellin345] | | | bacterium Ellin345 | |
| 131, | endoglucanase. | 228944 | 5.00E−72 | Prevotella ruminicola | P. pabuli xyloglucanase XYG1022 DNA |
| 132 | | | | | amplifying PCR primer 189585. |
| 133, | endoglucanase - Clostridium cellulovorans. | 98588 | 1.00E−88 | Clostridium | Amino acid sequence of a CelE |
| 134 | | | | cellulovorans | cellulase polypeptide. |
| 135, | ENDOGLUCANASE F PRECURSOR (ENDO-1,4- | 1708081 | 0 | Clostridium | Clostridium josui cellulose degrading |
| 136 | BETA-GLUCANASE F) (CELLULASE F) (EGCCF). | | | cellulolyticum | cellulase D protein. |
| 137, | cellulose 1;4-beta-cellobiosidase [Streptomyces | 29828397 | 0 | Streptomyces | Bacterial polypeptide #10001. |
| 138 | avermitilis MA-4680] | | | avermitilis MA-4680 | |
| 139, | secreted cellulase [Streptomyces coelicolor A3(2)] | 21224848 | 0 | Streptomyces | Bacterial polypeptide #10001. |
| 140 | | | | coelicolor A3(2) | |
| 141, | endoglucanase. | 228944 | 7.00E−65 | Prevotella ruminicola | Cow cellulase DNA clones pBKRR 2 |
| 142 | | | | | and pBKRR 16 SEQ ID NO: 3. |
| 143, | endoglucanase. | 228944 | 6.00E−63 | Prevotella ruminicola | Cow cellulase DNA clones pBKRR 2 |
| 144 | | | | | and pBKRR 16 SEQ ID NO: 3. |
| 145, | cellulase [uncultured bacterium] | 56675038 | 1.00E−120 | uncultured bacterium | X campestris umce19A cellulase gene |
| 146 | | | | | SeqID1. |
| 147, | endoglucanase - Clostridium cellulovorans. | 98588 | 3.00E−88 | Clostridium | Sequence of modified xylanase cDNA |
| 148 | | | | cellulovorans | in clone pNX-Tac. |
| 149, | Endoglucanase family 5 [Clostridium acetobutylicum]. | 15894113 | 1.00E−81 | Clostridium | Clostridium josui cellulose degrading |
| 150 | | | | acetobutylicum | cellulase D protein. |
| 151, | endo-1;4-beta-D-glucanase [uncultured bacterium] | 78926855 | 1.00E−119 | uncultured bacterium | X campestris umce19A cellulase gene |
| 152 | | | | | SeqID1. |
| 153, | endoglucanase - Clostridium cellulovorans. | 98588 | 4.00E−88 | Clostridium | Amino acid sequence of a CelE |
| 154 | | | | cellulovorans | cellulase polypeptide. |
| 155, | endo-1;4-beta-D-glucanase [uncultured bacterium] | 78926855 | 1.00E−118 | uncultured bacterium | X campestris umce19A cellulase gene |
| 156 | | | | | SeqID1. |
| 157, | endoglucanase - Clostridium cellulovorans. | 98588 | 4.00E−88 | Clostridium | Amino acid sequence of a CelE |
| 158 | | | | cellulovorans | cellulase polypeptide. |
| 159, | cellulase [unidentified microorganism] | 82524122 | 1.00E−68 | unidentified | Cow cellulase DNA clones pBKRR 2 |
| 160 | | | | microorganism | and pBKRR 16 SEQ ID NO: 3. |
| 161, | ENDOGLUCANASE B PRECURSOR (ENDO-1,4- | 121816 | 6.00E−49 | Pseudomonas | Acremonium sp. wild-type cellulase. |
| 162 | BETA-GLUCANASE) (CELLULASE) (EGB). | | | fluorescens | |
| 163, | secreted cellulase [Streptomyces coelicolor A3(2)] | 21224850 | 0 | Streptomyces | Thermostable cellulase-E3 catalytic |
| 164 | | | | coelicolor A3(2) | domain. |
| 165, | cellulose 1;4-beta-cellobiosidase [Streptomyces | 29828395 | 0 | Streptomyces | Thermostable cellulase-E3 catalytic |
| 166 | avermitilis MA-4680] | | | avermitilis MA-4680 | domain. |
| 167, | endoglucanase - Clostridium cellulovorans. | 98588 | 1.00E−88 | Clostridium | Amino acid sequence of a CelE |
| 168 | | | | cellulovorans | cellulase polypeptide. |
| 169, | EXOGLUCANASE II PRECURSOR | 1708082 | 0 | Clostridium | Clostridium josui cellulose degrading |
| 170 | (EXOCELLOBIOHYDROLASE II) (1,4-BETA- | | | stercorarium | cellulase D protein. |
| | CELLOBIOHYDROLASE II) (AVICELASE II). | | | | |
| 171, | ENDOGLUCANASE F PRECURSOR (ENDO-1,4- | 1708081 | 0 | Clostridium | Clostridium josui cellulose degrading |
| 172 | BETA-GLUCANASE F) (CELLULASE F) (EGCCF). | | | cellulolyticum | cellulase D protein. |
| 173, | EXOGLUCANASE II PRECURSOR | 1708082 | 0 | Clostridium | Clostridium josui cellulose degrading |
| 174 | (EXOCELLOBIOHYDROLASE II) (1,4-BETA- | | | stercorarium | cellulase D protein. |
| | CELLOBIOHYDROLASE II) (AVICELASE II). | | | | |
| 175, | EXOGLUCANASE II PRECURSOR | 1708082 | 0 | Clostridium | Clostridium josui cellulose degrading |
| 176 | (EXOCELLOBIOHYDROLASE II) (1,4-BETA- | | | stercorarium | cellulase D protein. |
| | CELLOBIOHYDROLASE II) (AVICELASE II). | | | | |
| 177, | Cellulose-binding, family II, bacterial type: Fibronectin, | 88930607 | 0 | Acidothermus | A. cellulolyticus Gux1 protein FN_III |
| 178 | type III [Acidothermus cellulolyticus 11B] | | | cellulolyticus 11B | domain fragment. |
| | gi\|88913077\|gb\|EAR32512.1\|Cellulose-binding, family | | | | |
| | II, bacterial type: Fibronectin, type III [Acidothermus | | | | |
| | cellulolyticus 11B] | | | | |
| 179, | cellulose 1;4-beta-cellobiosidase [Streptomyces | 29828397 | 0 | Streptomyces | A. cellulolyticus Gux1 protein FN_III |
| 180 | avermitilis MA-4680] | | | avermitilis MA-4680 | domain fragment. |
| 181, | cellulose 1;4-beta-cellobiosidase [Streptomyces | 29828395 | 1.00E−147 | Streptomyces | Exo-cellobiohydrolase cbh1 catalytic |
| 182 | avermitilis MA-4680] | | | avermitilis MA-4680 | domain. |
| 183, | secreted cellulase [Streptomyces coelicolor A3(2)] | 21224850 | 0 | Streptomyces | Thermostable cellulase-E3 catalytic |
| 184 | | | | coelicolor A3(2) | domain. |
| 185, | cellulose 1;4-beta-cellobiosidase [Streptomyces | 29828395 | 0 | Streptomyces | Thermostable cellulase-E3 catalytic |
| 186 | avermitilis MA-4680] | | | avermitilis MA-4680 | domain. |
| 187, | EXOGLUCANASE II PRECURSOR | 1708082 | 0 | Clostridium | Clostridium josui cellulose degrading |
| 188 | (EXOCELLOBIOHYDROLASE II) (1,4-BETA- | | | stercorarium | cellulase D protein. |
| | CELLOBIOHYDROLASE II) (AVICELASE II). | | | | |
| 189, | cellulose 1;4-beta-cellobiosidase [Streptomyces | 29828397 | 0 | Streptomyces | A. cellulolyticus Gux1 protein FN_III |
| 190 | avermitilis MA-4680] | | | avermitilis MA-4680 | domain fragment. |
| 191, | Cellulase [Frankia sp. EAN1pec] | 68235421 | 2.00E−72 | Frankia sp. EAN1pec | Amino acid sequence of a gene down- |
| 192 | gi\|68196961\|gb\|EAN11335.1\|Cellulase [Frankia sp. | | | | regulated during carbon starvation. |
| | EAN1pec] | | | | |
| 193, | glycoside hydrolase, family 48 [Herpetosiphon | 113939770 | 0 | Herpetosiphon | A. cellulolyticus Gux1 protein FN_III |
| 194 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 195, | cellulose 1;4-beta-cellobiosidase [Streptomyces | 29828397 | 0 | Streptomyces | Bacterial polypeptide #10001. |
| 196 | avermitilis MA-4680] | | | avermitilis MA-4680 | |
| 197, | secreted endoglucanase [Streptomyces coelicolor | 21221288 | 2.00E−57 | Streptomyces | Amino acid sequence of a gene down- |
| 198 | A3(2)] | | | coelicolor A3(2) | regulated during carbon starvation. |
| 199, | secreted endoglucanase [Streptomyces coelicolor | 21221288 | 4.00E−57 | Streptomyces | Amino acid sequence of a gene down- |
| 200 | A3(2)] | | | coelicolor A3(2) | regulated during carbon starvation. |
| 201, | Cellulase [Acidothermus cellulolyticus 11B] | 88932594 | 1.00E−130 | Acidothermus | M. xanthus protein sequence, seq id |
| 202 | gi\|88911374\|gb\|EAR30819.1\|Cellulase [Acidothermus | | | cellulolyticus 11B | 9726. |
| | cellulolyticus 11B] | | | | |
| 203, | Cellulase [Acidothermus cellulolyticus 11B] | 88932594 | 1.00E−130 | Acidothermus | M. xanthus protein sequence, seq id |
| 204 | gi\|88911374\|gb\|EAR30819.1\|Cellulase [Acidothermus | | | cellulolyticus 11B | 9726. |
| | cellulolyticus 11B] | | | | |
| 205, | endo-1;4-beta-D-glucanase [uncultured bacterium] | 78926927 | 1.00E−130 | uncultured bacterium | X campestris umce19A cellulase gene |
| 206 | | | | | SeqID1. |
| 207, | cellulose 1;4-beta-cellobiosidase [Streptomyces | 29828397 | 0 | Streptomyces | A. cellulolyticus Gux1 protein FN_III |
| 208 | avermitilis MA-4680] | | | avermitilis MA-4680 | domain fragment. |
| 209, | Cellulase [Acidothermus cellulolyticus 11B] | 88932594 | 1.00E−119 | Acidothermus | A. gossypii/S. halstedii fusion construct |
| 210 | gi\|88911374\|gb\|EAR30819.1\|Cellulase [Acidothermus | | | cellulolyticus 11B | containing cellulase DNA. |
| | cellulolyticus 11B] | | | | |
| 211, | endo-1;4-beta-D-glucanase [uncultured bacterium] | 78926855 | 1.00E−134 | uncultured bacterium | X campestris umce19A cellulase gene |
| 212 | | | | | SeqID1. |
| 213, | endoglucanase - Clostridium cellulovorans. | 98588 | 3.00E−87 | Clostridium | Amino acid sequence of a CelE |
| 214 | | | | cellulovorans | cellulase polypeptide. |
| 215, | Cellulase [Mycobacterium vanbaalenii PYR-1] | 90204581 | 3.00E−76 | Mycobacterium | Amino acid sequence of a gene down- |
| 216 | gi\|90196633\|gb\|EAS23395.1\|Cellulase [Mycobacterium | | | vanbaalenii PYR-1 | regulated during carbon starvation. |
| | vanbaalenii PYR-1] | | | | |
| 217, | CelA [Mycobacterium avium subsp. paratuberculosis K- | 41406378 | 6.00E−63 | Mycobacterium avium | Amino acid sequence of a gene down- |
| 218 | 10] | | | subsp. | regulated during carbon starvation. |
| | | | | paratuberculosis K-10 | |
| 219, | CelA [Mycobacterium avium subsp. paratuberculosis K- | 41406378 | 3.00E−63 | Mycobacterium avium | Amino acid sequence of a gene down- |
| 220 | 10] | | | subsp. | regulated during carbon starvation. |
| | | | | paratuberculosis K-10 | |
| 221, | cellulose 1;4-beta-cellobiosidase [Streptomyces | 29828395 | 0 | Streptomyces | Thermostable cellulase-E3 catalytic |
| 222 | avermitilis MA-4680] | | | avermitilis MA-4680 | domain. |
| 223, | cellulose 1;4-beta-cellobiosidase [Streptomyces | 29828395 | 0 | Streptomyces | Thermostable cellulase-E3 catalytic |
| 224 | avermitilis MA-4680] | | | avermitilis MA-4680 | domain. |
| 225, | cellulose 1;4-beta-cellobiosidase [Streptomyces | 29828395 | 0 | Streptomyces | Thermostable cellulase-E3 catalytic |
| 226 | avermitilis MA-4680] | | | avermitilis MA-4680 | domain. |
| 227, | cellulose 1;4-beta-cellobiosidase [Streptomyces | 29828395 | 0 | Streptomyces | Thermostable cellulase-E3 catalytic |
| 228 | avermitilis MA-4680] | | | avermitilis MA-4680 | domain. |
| 229, | cellulose 1;4-beta-cellobiosidase [Streptomyces | 29828395 | 0 | Streptomyces | Thermostable cellulase-E3 catalytic |
| 230 | avermitilis MA-4680] | | | avermitilis MA-4680 | domain. |
| 231, | endoglucanase D. | 606791 | 0 | Fibrobacter | X campestris umce19A cellulase gene |
| 232 | | | | succinogenes | SeqID1. |
| 233, | endoglucanase. | 228944 | 1.00E−68 | Prevotella ruminicola | Cow cellulase DNA clones pBKRR 2 |
| 234 | | | | | and pBKRR 16 SEQ ID NO: 3. |
| 235, | CelA [Mycobacterium avium subsp. paratuberculosis K- | 41406378 | 2.00E−62 | Mycobacterium avium | Amino acid sequence of a gene down- |
| 236 | 10] | | | subsp. | regulated during carbon starvation. |
| | | | | paratuberculosis K-10 | |
| 237, | CelA [Mycobacterium avium subsp. paratuberculosis K- | 41406378 | 3.00E−62 | Mycobacterium avium | Amino acid sequence of a gene down- |
| 238 | 10] | | | subsp. | regulated during carbon starvation. |
| | | | | paratuberculosis K-10 | |
| 239, | CelA [Mycobacterium avium subsp. paratuberculosis K- | 41406378 | 2.00E−62 | Mycobacterium avium | Amino acid sequence of a gene down- |
| 240 | 10] | | | subsp. | regulated during carbon starvation. |
| | | | | paratuberculosis K-10 | |
| 241, | glycoside hydrolase, family 48 [Herpetosiphon | 113939770 | 0 | Herpetosiphon | A. cellulolyticus Gux1 protein FN_III |
| 242 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 243, | Cellulase [Acidothermus cellulolyticus 11B] | 88932594 | 1.00E−124 | Acidothermus | Saccharothrix australiensis endo-beta- |
| 244 | gi\|88911374\|gb\|EAR30819.1\|Cellulase [Acidothermus | | | cellulolyticus 11B | 1,4-glucanase gene. |
| | cellulolyticus 11B] | | | | |
| 245, | Cellulase [Saccharophagus degradans 2-40] | 90020283 | 1.00E−117 | Saccharophagus | Microbulbifer degradans cellulase |
| 246 | | | | degradans 2-40 | system protein - SEQ ID 8. |
| 247, | cellulase [uncultured bacterium] | 56675038 | 1.00E−116 | uncultured bacterium | X campestris umce19A cellulase gene |
| 248 | | | | | SeqID1. |
| 249, | endoglucanase - Clostridium cellulovorans. | 98588 | 5.00E−87 | Clostridium | Amino acid sequence of a CelE |
| 250 | | | | cellulovorans | cellulase polypeptide. |
| 251, | GnuB [uncultured bacterium] | 37222147 | 1.00E−46 | uncultured bacterium | Primer used to construct a hybrid |
| 252 | | | | | endoglucanase. |
| 253, | glycoside hydrolase, family 48 [Herpetosiphon | 113939770 | 0 | Herpetosiphon | A. cellulolyticus Gux1 protein FN_III |
| 254 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 255, | glycoside hydrolase, family 48 [Herpetosiphon | 113939770 | 0 | Herpetosiphon | A. cellulolyticus Gux1 protein FN_III |
| 256 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 257, | glycoside hydrolase, family 48 [Herpetosiphon | 113939770 | 0 | Herpetosiphon | A. cellulolyticus Gux1 protein FN_III |
| 258 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 259, | glycoside hydrolase, family 48 [Herpetosiphon | 113939770 | 0 | Herpetosiphon | A. cellulolyticus Gux1 protein FN_III |
| 260 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 261, | CMC-xylanase [Fibrobacter succinogenes S85]. | 2980984 | 1.00E−145 | Fibrobacter | Xylanase from an environmental |
| 262 | | | | succinogenes S85 | sample seq id 14. |
| 263, | glycoside hydrolase, family 48 [Herpetosiphon | 113939770 | 0 | Herpetosiphon | A. cellulolyticus Gux1 protein FN_III |
| 264 | aurantiacus ATCC 23779] | | | aurantiacus ATCC | domain fragment. |
| | gi\|113898624\|gb\|EAU17637.1\|glycoside hydrolase, | | | 23779 | |
| | family 48 [Herpetosiphon aurantiacus ATCC 23779] | | | | |
| 265, | hypothetical protein Cphamn1DRAFT_0678 | 67942301 | 9.00E−22 | Chlorobium | Prokaryotic essential gene #34740. |
| 266 | [Chlorobium phaeobacteroides BS1] | | | phaeobacteroides | |
| | gi\|67911488\|gb\|EAM61510.1\|hypothetical protein | | | BS1 | |
| | Cphamn1DRAFT_0678 [Chlorobium phaeobacteroides | | | | |
| | BS1] | | | | |
| 267, | exoglucanase 2 precursor [Aspergillus terreus NIH2624] | 115401052 | 0 | Aspergillus terreus | A. fumigatus AfGOX3. |
| 268 | | | | NIH2624 | |
| 269, | glycoside hydrolase; family 5 [Acidobacteria bacterium | 94968716 | 3.00E−40 | Acidobacteria | Glucose isomerase SEQ ID NO 20. |
| 270 | Ellin345] | | | bacterium Ellin345 | |
| 271, | endoglucanase 3. | 666885 | 3.00E−39 | Fibrobacter intestinalis | Glucose isomerase SEQ ID NO 20. |
| 272 | | | | | |
| 273, | beta-1,4-endoglucanase [Pratylenchus penetrans]. | 15777927 | 3.00E−59 | Pratylenchus | Bacterial polypeptide #10001. |
| 274 | | | | penetrans | |
| 275, | endoglucanase 3. | 666885 | 5.00E−39 | Fibrobacter intestinalis | Glucose isomerase SEQ ID NO 20. |
| 276 | | | | | |
| 277, | glycoside hydrolase; family 5 [Acidobacteria bacterium | 94968716 | 1.00E−39 | Acidobacteria | Glucose isomerase SEQ ID NO 20. |
| 278 | Ellin345] | | | bacterium Ellin345 | |
| 279, | endoglucanase 3. | 666885 | 6.00E−40 | Fibrobacter intestinalis | Glucose isomerase SEQ ID NO 20. |
| 280 | | | | | |
| 281, | GUNB_FUSOX Putative endoglucanase type B | 46115572 | 0 | Gibberella zeae PH-1 | Cellbionydrolase-2 (CBH2) mutant |
| 282 | precursor (Endo-1;4-beta-glucanase) (Cellulase) | | | | S316P. |
| | [Gibberella zeae PH-1] | | | | |
| 283, | beta-1,4-endoglucanase [Pratylenchus penetrans]. | 15777927 | 4.00E−59 | Pratylenchus | Bacterial polypeptide #10001. |
| 284 | | | | penetrans | |
| 285, | CHU large protein; endoglucanase; glycoside hydrolase | 110637516 | 2.00E−75 | Cytophaga | Bacterial polypeptide #10001. |
| 286 | family 5 protein [Cytophaga hutchinsonii ATCC 33406] | | | hutchinsonii ATCC | |
| | | | | 33406 | |
| 287, | Chitinase., Cellulase [Mycobacterium vanbaalenii PYR- | 90206181 | 0 | Mycobacterium | PCR primer, SP3R, used to amplify rice |
| 288 | 1] gi\|90194972\|gb\|EAS21741.1\|Chitinase., Cellulase | | | vanbaalenii PYR-1 | rbcS signal peptide. |
| | [Mycobacterium vanbaalenii PYR-1] | | | | |
| 289, | endoglucanase 3. | 666885 | 9.00E−41 | Fibrobacter intestinalis | Glucose isomerase SEQ ID NO 20. |
| 290 | | | | | |
| 291, | CELLODEXTRINASE. | 121818 | 9.00E−99 | Butyrivibrio | Microbulbifer degradans cellulase |
| 292 | | | | fibrisolvens | system protein - SEQ ID 8. |
| 293, | CELLODEXTRINASE. | 121818 | 1.00E−100 | Butyrivibrio | X campestris umce19A cellulase gene |
| 294 | | | | fibrisolvens | SeqID1. |
| 295, | endoglucanase D. | 606791 | 1.00E−132 | Fibrobacter | X campestris umce19A cellulase gene |
| 296 | | | | succinogenes | SeqID1. |
| 297, | cellulase [Xanthomonas campestris pv. campestris str. | 21231824 | 1.00E−133 | Xanthomonas | X campestris umce19A cellulase gene |
| 298 | ATCC 33913]. | | | campestris pv. | SeqID1. |
| | | | | campestris str. ATCC | |
| | | | | 33913 | |
| 299, | cellulase [Xanthomonas campestris pv. campestris str. | 21231824 | 1.00E−131 | Xanthomonas | X campestris umce19A cellulase gene |
| 300 | ATCC 33913]. | | | campestris pv. | SeqID1. |
| | | | | campestris str. ATCC | |
| | | | | 33913 | |
| 301, | endo-1;4-beta-D-glucanase [uncultured bacterium] | 78926927 | 1.00E−132 | uncultured bacterium | X campestris umce19A cellulase gene |
| 302 | | | | | SeqID1. |
| 303, | endoglucanase D. | 606791 | 1.00E−135 | Fibrobacter | X campestris umce19A cellulase gene |
| 304 | | | | succinogenes | SeqID1. |
| 305, | hypothetical protein Sde_3003 [Saccharophagus | 90022645 | 9.00E−62 | Saccharophagus | Microbulbifer degradans cellulase |
| 306 | degradans 2-40] | | | degradans 2-40 | system protein - SEQ ID 8. |
| 307, | hypothetical protein Sde_3003 [Saccharophagus | 90022645 | 1.00E−62 | Saccharophagus | Microbulbifer degradans cellulase |
| 308 | degradans 2-40] | | | degradans 2-40 | system protein - SEQ ID 8. |
| 309, | CELLODEXTRINASE. | 121818 | 2.00E−91 | Butyrivibrio | X campestris umce19A cellulase gene |
| 310 | | | | fibrisolvens | SeqID1. |
| 311, | hypothetical protein Sde_3003 [Saccharophagus | 90022645 | 4.00E−68 | Saccharophagus | Microbulbifer degradans cellulase |
| 312 | degradans 2-40] | | | degradans 2-40 | system protein - SEQ ID 8. |
| 313, | GnuB [uncultured bacterium] | 37222147 | 3.00E−35 | uncultured bacterium | Primer used to construct a hybrid |
| 314 | | | | | endoglucanase. |
| 315, | secreted cellulase [Streptomyces coelicolor A3(2)] | 21224850 | 0 | Streptomyces | Thermostable cellulase-E3 catalytic |
| 316 | | | | coelicolor A3(2) | domain. |
| 317, | cellobiohydrolase II-I [Volvariella volvacea] | 49333367 | 8.00E−87 | Volvariella volvacea | Trametes hirsuta cellulolytic enzyme- |
| 318 | | | | | related protein - SEQ ID 12. |
| 319, | cellobiohydrolase II-I [Volvariella volvacea] | 49333367 | 8.00E−87 | Volvariella volvacea | Trametes hirsuta cellulolytic enzyme- |
| 320 | | | | | related protein - SEQ ID 12. |
| 321, | cellobiohydrolase II-I [Volvariella volvacea] | 49333367 | 6.00E−87 | Volvariella volvacea | Trametes hirsuta cellulolytic enzyme- |
| 322 | | | | | related protein - SEQ ID 12. |
| 323, | endoglucanase A [Stigmatella aurantiaca DW4/3-1] | 115373264 | 2.00E−83 | Stigmatella aurantiaca | M. xanthus protein sequence, seq id |
| 324 | gi\|115369710\|gb\|EAU68645.1\|endoglucanase A | | | DW4/3-1 | 9726. |
| | [Stigmatella aurantiaca DW4/3-1] | | | | |
| 325, | endoglucanase A [Stigmatella aurantiaca DW4/3-1] | 115373264 | 2.00E−85 | Stigmatella aurantiaca | M. xanthus protein sequence, seq id |
| 326 | gi\|115369710\|gb\|EAU68645.1\|endoglucanase A | | | DW4/3-1 | 9726. |
| | [Stigmatella aurantiaca DW4/3-1] | | | | |
| 327, | endoglucanase A [Stigmatella aurantiaca DW4/3-1] | 115373264 | 2.00E−83 | Stigmatella aurantiaca | M. xanthus protein sequence, seq id |
| 328 | gi\|115369710\|gb\|EAU68645.1\|endoglucanase A | | | DW4/3-1 | 9726. |
| | [Stigmatella aurantiaca DW4/3-1] | | | | |
| 329, | endoglucanase A [Stigmatella aurantiaca DW4/3-1] | 115373264 | 9.00E−84 | Stigmatella aurantiaca | M. xanthus L protein sequence, seq id |
| 330 | gi\|115369710\|gb\|EAU68645.1\|endoglucanase A | | | DW4/3-1 | 9726. |
| | [Stigmatella aurantiaca DW4/3-1] | | | | |
| 331, | endoglucanase A [Stigmatella aurantiaca DW4/3-1] | 115373264 | 6.00E−85 | Stigmatella aurantiaca | M. xanthus protein sequence, seq id |
| 332 | gi\|115369710\|gb\|EAU68645.1\|endoglucanase A | | | DW4/3-1 | 9726. |
| | [Stigmatella aurantiaca DW4/3-1] | | | | |
| 333, | cellobiohydrolase II-I [Volvariella volvacea] | 49333367 | 1.00E−86 | Volvariella volvacea | Trametes hirsuta cellulolytic enzyme- |
| 334 | | | | | related protein - SEQ ID 12. |
| 335, | cellobiohydrolase II-I [Volvariella volvacea] | 49333367 | 8.00E−87 | Volvariella volvacea | Trametes hirsuta cellulolytic enzyme- |
| 336 | | | | | related protein - SEQ ID 12. |
| 337, | endo-1;4-beta-D-glucanase [uncultured bacterium] | 78926855 | 1.00E−134 | uncultured bacterium | X campestris umce19A cellulase gene |
| 338 | | | | | SeqID1. |
| 339, | endoglucanase-related protein; glycoside hydrolase | 110638631 | 1.00E−87 | Cytophaga | X campestris umce19A cellulase gene |
| 340 | family 9 protein [Cytophaga hutchinsonii ATCC 33406] | | | hutchinsonii ATCC | SeqID1. |
| | | | | 33406 | |
| 341, | beta-1,4-endoglucanase [Cellulomonas pachnodae]. | 5880498 | 1.00E−112 | Cellulomonas | Amino acid sequence of a gene down- |
| 342 | | | | pachnodae | regulated during carbon starvation. |
| 343, | beta-1,4-endoglucanase [Cellulomonas pachnodae]. | 5880498 | 1.00E−112 | Cellulomonas | Amino acid sequence of a gene down- |
| 344 | | | | pachnodae | regulated during carbon starvation. |
| 345, | glycoside hydrolase, family 6 [Herpetosiphon | 113938252 | 1.00E−146 | Herpetosiphon | Amino acid sequence of the GuxA |
| 346 | aurantiacus ATCC 23779] | | | aurantiacus ATCC | potential signal peptide. |
| | gi\|113900042\|gb\|EAU19035.1\|glycoside hydrolase, | | | 23779 | |
| | family 6 [Herpetosiphon aurantiacus ATCC 23779] | | | | |
| 347, | GnuB [uncultured bacterium] | 37222147 | 5.00E−56 | uncultured bacterium | Primer used to construct a hybrid |
| 348 | | | | | endoglucanase. |
| 349, | glycoside hydrolase, family 6 [Herpetosiphon | 113938252 | 1.00E−119 | Herpetosiphon | Microbulbifer degradans cellulase |
| 350 | aurantiacus ATCC 23779] | | | aurantiacus ATCC | system protein - SEQ ID 8. |
| | gi\|113900042\|gb\|EAU19035.1\|glycoside hydrolase, | | | 23779 | |
| | family 6 [Herpetosiphon aurantiacus ATCC 23779] | | | | |
| 351, | Cellobiohydrolase A (1 4-beta-cellobiosidase A)-like | 90021917 | 0 | Saccharophagus | Microbulbifer degradans cellulase |
| 352 | [Saccharophagus degradans 2-40] | | | degradans 2-40 | system protein - SEQ ID 8. |
| 353, | secreted endoglucanase [Streptomyces coelicolor | 21221288 | 7.00E−63 | Streptomyces | Saccharothrix australiensis endo-beta- |
| 354 | A3(2)] | | | coelicolor A3(2) | 1,4-glucanase gene. |
| 355, | cellobiohydrolase D [Aspergillus fumigatus Af293] | 70991503 | 0 | Aspergillus fumigatus | Cellobiohydrolase I activity protein SEQ |
| 356 | | | | Af293 | ID No 16. |
| 357, | EXOGLUCANASE II PRECURSOR | 121855 | 0 | Hypocrea jecorina | Cellbionydrolase-2 (CBH2) mutant |
| 358 | (EXOCELLOBIOHYDROLASE II) (CBHII) (1,4-BETA- | | | | S316P. |
| | CELLOBIOHYDROLASE). | | | | |
| 359, | cellobiohydrolase I [Penicillium occitanis] | 51243029 | 0 | Penicillium occitanis | Acremonium cellulolyticus xylanase |
| 360 | | | | | precursor. |
| 361, | Glycoside hydrolase, family 9: Bacterial type 3a | 67874739 | 0 | Clostridium | TokcelR primer used to isolate Tok7B.1 |
| 362 | cellulose-binding domain: Clostridium cellulosome | | | thermocellum ATCC | celE gene. |
| | enzyme, dockerin type I [Clostridium thermocellum | | | 27405 | |
| | ATCC 27405] gi\|121828\|sp\|P26224\|GUNF_CLOTM | | | | |
| | Endoglucanase F precursor (EGF) (Endo-1,4-beta- | | | | |
| | glucanase) (Cellul | | | | |
| 363, | Glycoside hydrolase, family 18: Clostridium cellulosome | 67873373 | 0 | Clostridium | Thermus aquaticus Taq polymerase |
| 364 | enzyme, dockerin type I [Clostridium thermocellum | | | thermocellum ATCC | homolog No. 3. |
| | ATCC 27405] gi\|67851769\|gb\|EAM47332.1\|Glycoside | | | 27405 | |
| | hydrolase, family 18: Clostridium cellulosome enzyme, | | | | |
| | dockerin type I [Clostridium thermocellum ATCC 2 | | | | |
| 365, | Glycoside hydrolase, family 8: Clostridium cellulosome | 67873374 | 0 | Clostridium | Clostridium josui cellulose degrading |
| 366 | enzyme, dockerin type I [Clostridium thermocellum | | | thermocellum ATCC | cellulase D protein. |
| | ATCC 27405] gi\|121803\|sp\|P04955\|GUNA_CLOTM | | | 27405 | |
| | Endoglucanase A precursor (EGA) (Endo-1,4-beta- | | | | |
| | glucanase) (Cellulase A) gi\|144753\|gb\|AAA83521.1\| | | | | |
| | endoglucanase | | | | |
| 367, | endo-1;4-beta-D-glucanase [uncultured bacterium] | 78926855 | 1.00E−119 | uncultured bacterium | X campestris umce19A cellulase gene |
| 368 | | | | | SeqID1. |
| 431, | endo-1;4-beta-xylanase precursor [uncultured | 46253618 | 2.00E−93 | uncultured bacterium | Xylanase from an environmental |
| 432 | bacterium] | | | | sample seq id 14. |
| 433, | ENDO-1,4-BETA-XYLANASE B PRECURSOR | 139881 | 0 | Pseudomonas | Xylanase from an environmental |
| 434 | (XYLANASE B) (1,4-BETA-D-XYLAN | | | fluorescens | sample seq id 14. |
| | XYLANOHYDROLASE B). | | | | |
| 435, | endo-1,3(4)-beta-glucanase [Clostridium thermocellum]. | 19171141 | 1.00E−153 | Clostridium | Bacillus circulans oligonucleotide. |
| 436 | | | | thermocellum | |
| 437, | cellulase [Bacillus sp. BP-23]. | 4490766 | 5.00E−98 | Bacillus sp. BP-23 | Bacillus sp. KSM-N440 alkaline |
| 438 | | | | | cellulase protein, SEQ ID 4. |
| 439, | Glycoside hydrolase, family 10: Clostridium cellulosome | 67873837 | 1.00E−130 | Clostridium | Xylanase from an environmental |
| 440 | enzyme, dockerin type I: Carbohydrate-binding, CenC- | | | thermocellum ATCC | sample seq id 14. |
| | like [Clostridium thermocellum ATCC 27405] | | | 27405 | |
| | gi\|67851540\|gb\|EAM47104.1\|Glycoside hydrolase, | | | | |
| | family 10: Clostridium cellulosome enzyme, dockerin | | | | |
| | type I: | | | | |
| 441, | xylanase XynA GH 10 [Paenibacillus sp. JDR-2] | 62990090 | 8.00E−97 | Paenibacillus sp. JDR-2 | Xylanase from an environmental |
| 442 | | | | | sample seq id 14. |
| 443, | Putative esterase: Glycoside hydrolase, family | 67916212 | 0 | Clostridium | Xylanase from an environmental |
| 444 | 10: Clostridium cellulosome enzyme, dockerin type | | | thermocellum ATCC | sample seq id 14. |
| | I: Carbohydrate-binding, CenC-like [Clostridium | | | 27405 | |
| | thermocellum ATCC 27405] | | | | |
| | gi\|67849815\|gb\|EAM45408.1\|Putative | | | | |
| | esterase: Glycoside hydrolase, family 10: Clostridium | | | | |
| 445, | glycoside hydrolase, family 9 [Herpetosiphon | 113939769 | 0 | Herpetosiphon | Vibrio harveyi endoglucanase DNA. |
| 446 | aurantiacus ATCC 23779] | | | aurantiacus ATCC | |
| | gi\|113898623\|gb\|EAU17636.1\|glycoside hydrolase, | | | 23779 | |
| | family 9 [Herpetosiphon aurantiacus ATCC 23779] | | | | |
| 447, | beta-glucanase [thermophilic anaerobe NA10]. | 2564015 | 0 | thermophilic anaerobe | TokcelR primer used to isolate Tok7B.1 |
| 448 | | | | NA10 | celE gene. |
| 449, | cellulose binding protein CelS2 [Streptomyces | 4680329 | 0 | Streptomyces | Pseudomonas aeruginosa quorum |
| 450 | viridosporus]. | | | viridosporus | sensing controlled protein, SEQ ID 399. |
| 451, | uncharacterized protein contain chitin-binding domain | 83644003 | 1.00E−151 | Hahella chejuensis | Enterobacter cloacae protein amino |
| 452 | type 3 [Hahella chejuensis KCTC 2396] | | | KCTC 2396 | acid sequence - SEQ ID 5666. |
| 453, | hypothetical protein Acid_6287 [Solibacter usitatus | 116625342 | 2.00E−19 | Solibacter usitatus | Xylanase from an environmental |
| 454 | Ellin6076] gi\|116228504\|gb\|ABJ87213.1\|hypothetical | | | Ellin6076 | sample seq id 14. |
| | protein Acid_6287 [Solibacter usitatus Ellin6076] | | | | |
| 455, | cellulose-binding; family II; bacterial type [Thermobifida | 72161048 | 1.00E−150 | Thermobifida fusca | Bacterial polypeptide #10001. |
| 456 | fusca YX] | | | YX | |
| 457, | cellulose-binding; family II; bacterial type: Fibronectin; | 72162066 | 0 | Thermobifida fusca | Pseudomonas aeruginosa quorum |
| 458 | type III [Thermobifida fusca YX] | | | YX | sensing controlled protein, SEQ ID 399. |
| 459, | chitin-binding protein [Streptomyces thermoviolaceus] | 38347733 | 4.00E−80 | Streptomyces | Enterobacter cloacae protein amino |
| 460 | | | | thermoviolaceus | acid sequence - SEQ ID 5666. |
| 461, | laminarinase [Thermotoga maritima]. | 15642799 | 0 | Thermotoga maritima | Oerskovia xanthineolytica beta-1,3- |
| 462 | | | | | glucanase. |
| 471, | secreted cellulose binding protein [Streptomyces | 21219699 | 0 | Streptomyces | Pseudomonas aeruginosa quorum |
| 472 | coelicolor A3(2)] | | | coelicolor A3(2) | sensing controlled protein, SEQ ID 399. |
| 489, | hypothetical protein FG03795.1 [Gibberella zeae PH-1] | 46115906 | 1.00E−163 | Gibberella zeae PH-1 | P. brasilianum cel5c endoglucanase |
| 490 | | | | | reverse PCR primer, SEQ ID NO: 15. |
| 491, | endoglucanase C [Aspergillus kawachii]. | 15054480 | 0 | Aspergillus kawachii | Endo beta-1,4-gluconase peptide 3. |
| 492 | | | | | |
| 493, | endoglucanase C [Aspergillus kawachii]. | 15054480 | 0 | Aspergillus kawachii | Endo beta-1,4-gluconase peptide 3. |
| 494 | | | | | |
| 495, | hypothetical protein SNOG_04886 [Phaeosphaeria | 1.61E+08 | 1.00E−160 | Phaeosphaeria | Cellulase cDNA clone 12. |
| 496 | nodorum SN15] | | | nodorum SN15 | |
| 497, | hypothetical protein FG03795.1 [Gibberella zeae PH-1] | 46115906 | 1.00E−157 | Gibberella zeae PH-1 | Bacterial polypeptide #23667. |
| 498 | | | | | |
| 499, | hypothetical protein FG03795.1 [Gibberella zeae PH-1] | 46115906 | 0 | Gibberella zeae PH-1 | P. brasilianum cel5c endoglucanase |
| 500 | | | | | reverse PCR primer, SEQ ID NO: 15. |
| 501, | hypothetical protein [Neurospora crassa OR74A] | 85111901 | 1.00E−158 | Neurospora crassa | Bacterial polypeptide #23667. |
| 502 | gi\|28925928\|gb\|EAA34923.1\|endoglucanase 3 | | | OR74A | |
| | precursor [Neurospora crassa OR74A] | | | | |
| | gi\|38636418\|emb\|CAE81955.1\|probable cellulase | | | | |
| | precursor [Neurospora crassa] | | | | |
| 503, | hypothetical protein FG01621.1 [Gibberella zeae PH-1] | 46109478 | 1.00E−119 | Gibberella zeae PH-1 | Endoglucanase protein. |
| 504 | | | | | |
| 505, | hypothetical protein CHGG_01188 [Chaetomium | 1.16E+08 | 1.00E−179 | Chaetomium | Endoglucanase SEQ ID NO: 6. |
| 506 | globosum CBS 148.51] gi\|88185485\|gb\|EAQ92953.1\| | | | globosum CBS | |
| | hypothetical protein CHGG_01188 [Chaetomium | | | 148.51 | |
| | globosum CBS 148.51] | | | | |
| 507, | hypothetical protein CHGG_02213 [Chaetomium | 1.16E+08 | 1.00E−124 | Chaetomium | Talaromyces emersonii beta- |
| 508 | globosum CBS 148.51] gi\|88182810\|gb\|EAQ90278.1\| | | | globosum CBS | glucanase CEC protein. |
| | hypothetical protein CHGG_02213 [Chaetomium | | | 148.51 | |
| | globosum CBS 148.51] | | | | |
| 509, | ENDOGLUCANASE 3 PRECURSOR (ENDO-1,4- | 13959390 | 0 | Humicola insolens | Endoglucanase SEQ ID NO: 6. |
| 510 | BETA-GLUCANASE 3) (CELLULASE 3). | | | | |
| 511, | hypothetical protein An01g11670 [Aspergillus niger] | 1.45E+08 | 0 | Aspergillus niger | P. brasilianum cel5c endoglucanase |
| 512 | gi\|134055695\|emb\|CAK44069.1\|unnamed protein | | | | reverse PCR primer, SEQ ID NO: 15. |
| | product [Aspergillus niger] | | | | |
| 513, | hypothetical protein FG03795.1 [Gibberella zeae PH-1] | 46115906 | 0 | Gibberella zeae PH-1 | P. brasilianum cel5c endoglucanase |
| 514 | | | | | reverse PCR primer, SEQ ID NO: 15. |
| 515, | glycoside hydrolase, family 5 [Clostridium thermocellum | 1.26E+08 | 0 | Clostridium | Orpinomyces cellulase CelB cDNA. |
| 516 | ATCC 27405] gi\|125713540\|gb\|ABN52032.1\|glycoside | | | thermocellum ATCC | |
| | hydrolase, family 5 [Clostridium thermocellum ATCC | | | 27405 | |
| | 27405] | | | | |
| 517, | hypothetical protein An01g11670 [Aspergillus niger] | 1.45E+08 | 0 | Aspergillus niger | P. brasilianum cel5c endoglucanase |
| 518 | gi\|134055695\|emb\|CAK44069.1\|unnamed protein | | | | reverse PCR primer, SEQ ID NO: 15. |
| | product [Aspergillus niger] | | | | |
| 519, | endoglucanase, putative [Aspergillus fumigatus Af293] | 70992389 | 1.00E−170 | Aspergillus fumigatus | P. brasilianum cel5c endoglucanase |
| 520 | gi\|66848676\|gb\|EAL89005.1\|endoglucanase, putative | | | Af293 | reverse PCR primer, SEQ ID NO: 15. |
| | [Aspergillus fumigatus Af293] | | | | |
| 521, | hypothetical protein An12g02220 [Aspergillus niger] | 1.45E+08 | 0 | Aspergillus niger | A. fumigatus AfGOX3. |
| 522 | gi\|134080021\|emb\|CAK41068.1\|unnamed protein | | | | |
| | product [Aspergillus niger] | | | | |
| 523, | cellulose 1,4-beta-cellobiosidase [Acremonium | 1.57E+08 | 0 | Acremonium | Cellobiohydrolase I activity protein |
| 524 | thermophilum] | | | thermophilum | SEQ ID No 16. |
| 525, | Beta-glucosidase [Maricaulis maris MCS10] | 1.15E+08 | 0 | Maricaulis maris | Microbulbifer degradans cellulase |
| 526 | gi\|114341732\|gb\|ABI67012.1\|exo-1,4-beta-glucosidase | | | MCS10 | system protein - SEQ ID 8. |
| | [Maricaulis maris MCS10] | | | | |
| 527, | Beta-N-acetylglucosaminidase/beta-glucosidase (3- | 75387204 | 1.00E−147 | Cellulomonas fimi | Bacterial beta-hexosaminidase gene |
| 528 | beta-N-acetyl-D-glucosaminidase/beta-D-glucosidase) | | | | SEQ ID NO: 8. |
| | (Nag3) gi\|33320077\|gb\|AAQ05801.1\|AF478460_1 N- | | | | |
| | acetyl-beta-glucosaminidase [Cellulomonas fimi] | | | | |
| 529, | BETA-GLUCOSIDASE A (GENTIOBIASE) | 114957 | 0 | Clostridium | Agrobacterium sp. bgls_agrsp strand- |
| 530 | (CELLOBIASE) (BETA-D-GLUCOSIDE | | | thermocellum | glucosidase. |
| | GLUCOHYDROLASE). | | | | |
| 531, | Beta-glucosidase [Sorangium cellulosum ‘So ce 56’] | 1.62E+08 | 1.00E−154 | Sorangium | Bacterial polypeptide #23667. |
| 532 | gi\|161163155\|emb\|CAN94460.1\|Beta-glucosidase | | | cellulosum ‘So ce 56 | |
| | [Sorangium cellulosum ‘So ce 56’] | | | | |
| 533, | hypothetical protein RUMOBE_00331 [Ruminococcus | 1.54E+08 | 1.00E−121 | Ruminococcus | Anti-biofilm polypeptide #100. |
| 534 | obeum ATCC 29174] gi\|149834128\|gb\|EDM89208.1\| | | | obeum ATCC 29174 | |
| | hypothetical protein RUMOBE_00331 [Ruminococcus | | | | |
| | obeum ATCC 29174] | | | | |
| 535, | beta-glucosidase [Pyrococcus horikoshii]. | 14590274 | 0 | Pyrococcus horikoshii | Anti-biofilm polypeptide #100. |
| 536 | | | | | |
| 537, | beta-glucosidase [Thermotoga maritima]. | 15642800 | 0 | Thermotoga maritima | Anti-biofilm polypeptide #100. |
| 538 | | | | | |
| 539, | Beta-glucosidase [Sorangium cellulosum ‘So ce 56’] | 1.62E+08 | 0 | Sorangium | Anti-biofilm polypeptide #100. |
| 540 | gi\|161166527\|emb\|CAN97832.1\|Beta-glucosidase | | | cellulosum ‘So ce 56 | |
| | [Sorangium cellulosum ‘So ce 56’] | | | | |
| 541, | glycoside hydrolase family 1 [Opitutaceae bacterium | 1.54E+08 | 1.00E−177 | Opitutaceae | Anti-biofilm polypeptide #100. |
| 542 | TAV2] gi\|151582326\|gb\|EDN45879.1\|glycoside | | | bacterium TAV2 | |
| | hydrolase family 1 [Opitutaceae bacterium TAV2] | | | | |
| 543, | glycoside hydrolase family 1 [Chloroflexus aurantiacus | 1.64E+08 | 1.00E−125 | Chloroflexus | Bacterial polypeptide #23667. |
| 544 | J-10-fl] gi\|163667244\|gb\|ABY33610.1\|glycoside | | | aurantiacus J-10-fl | |
| | hydrolase family 1 [Chloroflexus aurantiacus J-10-fl] | | | | |
| 545, | Beta-glucosidase [Salinispora arenicola CNS-205] | 1.59E+08 | 1.00E−129 | Salinispora arenicola | T. bispora NRRL 15568 beta- |
| 546 | gi\|157914892\|gb\|ABV96319.1\|Beta-glucosidase | | | CNS-205 | glucosidase. |
| | [Salinispora arenicola CNS-205] | | | | |
| 547, | Beta-glucosidase [Sorangium cellulosum ‘So ce 56’] | 1.62E+08 | 0 | Sorangium | Bacterial polypeptide #23667. |
| 548 | gi\|161163155\|emb\|CAN94460.1\|Beta-glucosidase | | | cellulosum ‘So ce 56 | |
| | [Sorangium cellulosum ‘So ce 56’] | | | | |
| 549, | beta-glucosidase [Vibrio shilonii AK1] | 1.49E+08 | 1.00E−163 | Vibrio shilonii AK1 | Anti-biofilm polypeptide #100. |
| 550 | gi\|148838481\|gb\|EDL55421.1\|beta-glucosidase [Vibrio | | | | |
| | shilonii AK1] | | | | |
| 551, | glycoside hydrolase, family 1 [Novosphingobium | 87198566 | 1.00E−122 | Novosphingobium | Bacterial polypeptide #23667. |
| 552 | aromaticivorans DSM 12444] | | | aromaticivorans DSM | |
| | gi\|87134247\|gb\|ABD24989.1\|glycoside hydrolase, | | | 12444 | |
| | family 1 [Novosphingobium aromaticivorans DSM | | | | |
| | 12444] | | | | |
| 553, | beta-glucosidase [Pyrococcus horikoshii]. | 14590274 | 2.00E−77 | Pyrococcus horikoshii | Pyrococcus horikoshii beta- |
| 554 | | | | | glycosidase enzyme - SEQ ID 2. |
| 555, | beta-glucosidase [Pyrococcus furiosus DSM 3638]. | 18976445 | 0 | Pyrococcus furiosus | Thermostable beta-galactosidase |
| 556 | | | | DSM 3638 | conserved sequence (Box 10). |
| 557, | hypothetical protein SNOG_12988 [Phaeosphaeria | 1.61E+08 | 0 | Phaeosphaeria | Trichoderma reesei bgl1 gene. |
| 558 | nodorum SN15] | | | nodorum SN15 | |
| 559, | glycoside hydrolase family 1 [Fervidobacterium | 1.54E+08 | 0 | Fervidobacterium | Anti-biofilm polypeptide #100. |
| 560 | nodosum Rt17-B1] gi\|154154169\|gb\|ABS61401.1\| | | | nodosum Rt17-B1 | |
| | glycoside hydrolase family 1 [Fervidobacterium | | | | |
| | nodosum Rt17-B1] | | | | |
| 561, | putative Beta-glucosidase A [Loktanella vestfoldensis | 84517375 | 1.00E−178 | Loktanella | T. bispora NRRL 15568 beta- |
| 562 | SKA53] gi\|84508739\|gb\|EAQ05203.1\|putative Beta- | | | vestfoldensis SKA53 | glucosidase. |
| | glucosidase A [Loktanella vestfoldensis SKA53] | | | | |
| 563, | Beta-glucosidase [Sorangium cellulosum ‘So ce 56’] | 1.62E+08 | 0 | Sorangium | Anti-biofilm polypeptide #100. |
| 564 | gi\|161166527\|emb\|CAN97832.1\|Beta-glucosidase | | | cellulosum ‘So ce 56 | |
| | [Sorangium cellulosum ‘So ce 56’] | | | | |
| 565, | Beta-glucosidase [Roseiflexus sp. RS-1] | 1.49E+08 | 1.00E−153 | Roseiflexus sp. RS-1 | Bacterial polypeptide #23667. |
| 566 | gi\|148569824\|gb\|ABQ91969.1\|beta-glucosidase. | | | | |
| | Glycosyl Hydrolase family 1. [Roseiflexus sp. RS-1] | | | | |
| 567, | RNA-binding protein [Cytophaga hutchinsonii ATCC | 1.11E+08 | 8.00E−40 | Cytophaga | Protein encoded by Prokaryotic |
| 568 | 33406] gi\|110281863\|gb\|ABG60049.1\|RNA-binding | | | hutchinsonii ATCC | essential gene #30232. |
| | protein [Cytophaga hutchinsonii ATCC 33406] | | | 33406 | |
| 569, | putative Beta-glucosidase A [Loktanella vestfoldensis | 84517375 | 0 | Loktanella | T. bispora NRRL 15568 beta- |
| 570 | SKA53] gi\|84508739\|gb\|EAQ05203.1\|putative Beta- | | | vestfoldensis SKA53 | glucosidase. |
| | glucosidase A [Loktanella vestfoldensis SKA53] | | | | |
| 571, | beta-glucosidase [Vibrio shilonii AK1] | 1.49E+08 | 1.00E−162 | Vibrio shilonii AK1 | Anti-biofilm polypetide #100. |
| 572 | gi\|148838481\|gb\|EDL55421.1\|beta-glucosidase [Vibrio | | | | |
| | shilonii AK1] | | | | |
| 573, | glycoside hydrolase, family 3-like [Acidobacteria | 94971178 | 1.00E−165 | Acidobacteria | Bacteroides fragilis strain 14062 |
| 574 | bacterium Ellin345] gi\|94553228\|gb\|ABF43152.1\| | | | bacterium Ellin345 | protein, SEQ: 5227. |
| | glycoside hydrolase, family 3-like [Acidobacteria | | | | |
| | bacterium Ellin345] | | | | |
| 575, | Beta-glucosidase [Thermoanaerobacter ethanolicus | 76795388 | 1.00E−147 | Thermoanaerobacter | Agrobacterium sp. bgls_agrsp strand- |
| 576 | ATCC 33223] gi\|76589196\|gb\|EAO65595.1\|Beta- | | | ethanolicus ATCC | glucosidase. |
| | glucosidase [Thermoanaerobacter ethanolicus ATCC | | | 33223 | |
| | 33223] | | | | |
| 577, | b-glucosidase, glycoside hydrolase family 3 protein | 1.49E+08 | 0 | Pedobacter sp. | Protein encoded by Prokaryotic |
| 578 | [Pedobacter sp. BAL39] gi\|149229614\|gb\|EDM35004.1\| | | | BAL39 | essential gene #30232. |
| | b-glucosidase, glycoside hydrolase family 3 protein | | | | |
| | [Pedobacter sp. BAL39] | | | | |
| 579, | glycoside hydrolase, family 1 [Salinispora tropica CNB- | 1.46E+08 | 0 | Salinispora tropica | T. bispora NRRL 15568 beta- |
| 580 | 440] gi\|145303444\|gb\|ABP54026.1\|beta-glucosidase. | | | CNB-440 | glucosidase. |
| | Glycosyl Hydrolase family 1. [Salinispora tropica CNB- | | | | |
| | 440] | | | | |
| 581, | glycoside hydrolase family 3 domain protein [Clostridium | 1.61E+08 | 0 | Clostridium | Enterococcus faecalis polypeptide #1. |
| 582 | phytofermentans ISDg] gi\|160427523\|gb\|ABX41086.1\| | | | phytofermentans | |
| | glycoside hydrolase family 3 domain protein [Clostridium | | | ISDg | |
| | phytofermentans ISDg] | | | | |
| 583, | glucan 1,4-beta-glucosidase precursor [Xanthomonas | 78047379 | 0 | Xanthomonas | Microbulbifer degradans cellulase |
| 584 | campestris pv. vesicatoria str. 85-10] | | | campestris pv. | system protein - SEQ ID 8. |
| | gi\|78035809\|emb\|CAJ23500.1\|glucan 1,4-beta- | | | vesicatoria str. 85-10 | |
| | glucosidase precursor [Xanthomonas campestris pv. | | | | |
| | vesicatoria str. 85-10] | | | | |
| 585, | b-glucosidase, glycoside hydrolase family 3 protein | 1.49E+08 | 0 | Pedobacter sp. | Bacteroides fragilis strain 14062 |
| 586 | [Pedobacter sp. BAL39] gi\|149229614\|gb\|EDM35004.1\| | | | BAL39 | protein, SEQ: 5227. |
| | b-glucosidase, glycoside hydrolase family 3 protein | | | | |
| | [Pedobacter sp. BAL39] | | | | |
| 587, | Beta-glucosidase [Caulobacter sp. K31] | 1.14E+08 | 0 | Caulobacter sp. K31 | Chimaeric thermostable beta- |
| 588 | gi\|113730277\|gb\|EAU11349.1\|Beta-glucosidase | | | | glucosidase. |
| | [Caulobacter sp. K31] | | | | |
| 589, | glycoside hydrolase, family 1 [Solibacter usitatus | 1.17E+08 | 1.00E−131 | Solibacter usitatus | Anti-biofilm polypeptide #100. |
| 590 | Ellin6076] gi\|116225047\|gb\|ABJ83756.1\|glycoside | | | Ellin6076 | |
| | hydrolase, family 1 [Solibacter usitatus Ellin6076] | | | | |
| 591, | Glycoside hydrolase, family 1 [Halothermothrix orenii H | 89211521 | 1.00E−117 | Halothermothrix orenii | Agrobacterium sp. bgls_agrsp strand- |
| 592 | 168] gi\|89158859\|gb\|EAR78546.1\|Glycoside hydrolase, | | | H 168 | glucosidase. |
| | family 1 [Halothermothrix orenii H 168] | | | | |
| 593, | Beta-glucosidase [Burkholderia sp. 383] | 78059828 | 0 | Burkholderia sp. 383 | Bacterial beta-hexosaminidase gene |
| 594 | gi\|77964378\|gb\|ABB05759.1\|Beta-glucosidase | | | | SEQ ID NO: 8. |
| | [Burkholderia sp. 383] | | | | |
| 595, | candidate b-glucosidase, Glycoside Hydrolase Family 3 | 1.64E+08 | 0 | Flavobacteriales | Bacteroides fragilis strain 14062 |
| 596 | protein [Flavobacteriales bacterium ALC-1] | | | bacterium ALC-1 | protein, SEQ: 5227. |
| | gi\|159877302\|gb\|EDP71359.1\|candidate b-glucosidase, | | | | |
| | Glycoside Hydrolase Family 3 protein [Flavobacteriales | | | | |
| | bacterium ALC-1] | | | | |
| 597, | EXOGLUCANASE II PRECURSOR | 121855 | 0 | Hypocrea jecorina | Hypocrea jecorina cellbionydrolase-2 |
| 598 | (EXOCELLOBIOHYDROLASE II) (CBHII) (1,4-BETA- | | | | (CBH2) SEQ ID NO 2. |
| | CELLOBIOHYDROLASE). | | | | |
| 599, | Exoglucanase 1 precursor (Exoglucanase I) | 50400675 | 0 | Trichoderma | PCR primer Mcbh1-N of the |
| 600 | (Exocellobiohydrolase I) (CBHI) (1,4-beta- | | | harzianum | specification. |
| | cellobiohydrolase) | | | | |
| | gi\|7107367\|gb\|AAF36391.1\|AF223252_1 | | | | |
| | cellobiohydrolase [Trichoderma harzianum] | | | | |
| 601, | hypothetical protein An12g02220 [Aspergillus niger] | 1.45E+08 | 0 | Aspergillus niger | A. fumigatus AfGOX3. |
| 602 | gi\|134080021\|emb\|CAK41068.1\|unnamed protein | | | | |
| | product [Aspergillus niger] | | | | |
| 603, | cellulose 1,4-beta-cellobiosidase [Acremonium | 1.57E+08 | 0 | Acremonium | Cellobiohydrolase I activity protein |
| 604 | thermophilum] | | | thermophilum | SEQ ID No 16. |
| 605, | EXOGLUCANASE I PRECURSOR | 729650 | 0 | Penicillium | Cellobiohydrolase I activity protein |
| 606 | (EXOCELLOBIOHYDROLASE I) (1,4-BETA- | | | janthinellum | SEQ ID No 16. |
| | CELLOBIOHYDROLASE). | | | | |
| 607, | hypothetical protein MGG_07809 [Magnaporthe grisea | 39973029 | 0 | Magnaporthe grisea | Cellobiohydrolase I activity protein |
| 608 | 70-15] gi\|145012585\|gb\|EDJ97239.1\|hypothetical | | | 70-15 | SEQ ID No 16. |
| | protein MGG_07809 [Magnaporthe grisea 70-15] | | | | |
| 609, | unnamed protein product [Aspergillus oryzae]. | 83770909 | 0 | Aspergillus oryzae | EP-897667 Seq ID 7. |
| 610 | | | | | |
| 611, | secreted hydrolase [Streptomyces coelicolor A3(2)] | 21224131 | 1.00E−116 | Streptomyces | Hypocrea jecorina AXE2 protein |
| 612 | gi\|2995294\|emb\|CAA18323.1\|putative secreted | | | coelicolor A3(2) | sequence SeqID15. |
| | hydrolase [Streptomyces coelicolor A3(2)] | | | | |
| 613, | endo-1,4-beta-glucanase b [Pyrococcus furiosus DSM | 18977226 | 1.00E−103 | Pyrococcus furiosus | Glucose isomerase SEQ ID NO 20. |
| 614 | 3638]. | | | DSM 3638 | |
| 615, | PUTATIVE EXOGLUCANASE TYPE C PRECURSOR | 1170141 | 0 | Fusarium oxysporum | Linking B region #8 derived from a |
| 616 | (EXOCELLOBIOHYDROLASE I) (1,4-BETA- | | | | (hemi)cellulose-degrading enzyme. |
| | CELLOBIOHYDROLASE) (BETA- | | | | |
| | GLUCANCELLOBIOHYDROLASE). | | | | |
| 617, | cellobiohydrolase [Irpex lacteus]. | 46395332 | 0 | Irpex lacteus | Cellobiohydrolase I activity protein |
| 618 | | | | | SEQ ID No 16. |
| 619, | cellobiohydrolase, putative [Aspergillus fumigatus Af293] | 70986018 | 0 | Aspergillus fumigatus | A. fumigatus AfGOX3. |
| 620 | gi\|66846140\|gb\|EAL86473.1\|cellobiohydrolase, putative | | | Af293 | |
| | [Aspergillus fumigatus Af293] | | | | |
| 621, | xylosidase/arabinosidase [Caulobacter crescentus]. | 16127284 | 0 | Caulobacter | Vibrio harveyi endoglucanase DNA. |
| 622 | | | | crescentus | |
| 623, | Beta-lactamase [Algoriphagus sp. PR1] | 1.27E+08 | 2.00E−97 | Algoriphagus sp. PR1 | Environmental isolate hydrolase, SEQ |
| 624 | gi\|126576725\|gb\|EAZ80973.1\|Beta-lactamase | | | | ID NO: 44. |
| | [Algoriphagus sp. PR1] | | | | |
| 625, | glycoside hydrolase, family 3 domain protein [Solibacter | 1.17E+08 | 0 | Solibacter usitatus | Vibrio harveyi endoglucanase DNA. |
| 626 | usitatus Ellin6076] gi\|116224959\|gb\|ABJ83668.1\| | | | Ellin6076 | |
| | glycoside hydrolase, family 3 domain protein [Solibacter | | | | |
| | usitatus Ellin6076] | | | | |
| 627, | hypothetical protein SNOG_01776 [Phaeosphaeria | 1.11E+08 | 1.00E−127 | Phaeosphaeria | Aspergillus fumigatus xylanase |
| 628 | nodorum SN15] | | | nodorum SN15 | mature protein #1. |
| 629, | endoxylanase [Alternaria alternata]. | 6179887 | 1.00E−140 | Alternaria alternata | Humicola insolens GH43 alpha-L- |
| 630 | | | | | arabinofuranosidase enzyme - SEQ |
| | | | | | ID 1. |
| 631, | hypothetical protein SNOG_08993 [Phaeosphaeria | 1.61E+08 | 0 | Phaeosphaeria | Aspergillus oryzae xylosidase. |
| 632 | nodorum SN15] | | | nodorum SN15 | |
| 633, | hypothetical protein SNOG_12988 [Phaeosphaeria | 1.61E+08 | 0 | Phaeosphaeria | Trichoderma reesei bgl1 gene. |
| 634 | nodorum SN15] | | | nodorum SN15 | |
| 635, | major extracellular beta-xylosidase [Cochliobolus | 3789946 | 0 | Cochliobolus | Microbulbifer degradans cellulase |
| 636 | carbonum]. | | | carbonum | system protein - SEQ ID 8. |
| 637, | hypothetical protein SNOG_00770 [Phaeosphaeria | 1.61E+08 | 0 | Phaeosphaeria | DNA encoding Aspergillus oryzae |
| 638 | nodorum SN15] | | | nodorum SN15 | endoglucanase. |
| 639, | Feruloyl esterase [Delftia acidovorans SPH-1] | 1.61E+08 | 7.00E−84 | Delftia acidovorans | Environmental isolate hydrolase, SEQ |
| 640 | gi\|160364556\|gb\|ABX36169.1\|Feruloyl esterase [Delftia | | | SPH-1 | ID NO: 44. |
| | acidovorans SPH-1] | | | | |
| 641, | hypothetical protein Mmcs_0784 [Mycobacterium sp. | 1.09E+08 | 2.00E−43 | Mycobacterium sp. | Environmental isolate hydrolase, SEQ |
| 642 | MCS] gi\|119866853\|ref\|YP_936805.1\|hypothetical | | | MCS | ID NO: 44. |
| | protein Mkms_0799 [Mycobacterium sp. KMS] | | | | |
| | gi\|108768182\|gb\|ABG06904.1\|hypothetical protein | | | | |
| | Mmcs_0784 [Mycobacterium sp. MCS] | | | | |
| | gi\|119692942\|gb\|ABL90015.1\|cons | | | | |
| 643, | Carboxylesterase, type B [Burkholderia phytofirmans | 1.18E+08 | 1.00E−112 | Burkholderia | Environmental isolate hydrolase, SEQ |
| 644 | PsJN] gi\|117992602\|gb\|EAV06893.1\|Carboxylesterase, | | | phytofirmans PsJN | ID NO: 44. |
| 645, | Beta-glucosidase [Sorangium cellulosum ‘So ce 56’] | 1.62E+08 | 1.00E−155 | Sorangium | Bacterial polypeptide #23667. |
| 646 | gi\|161163155\|emb\|CAN94460.1\|Beta-glucosidase | | | cellulosum ‘So ce 56 | |
| | [Sorangium cellulosum ‘So ce 56’] | | | | |
| 647, | alpha-glucuronidase [Xanthomonas campestris pv. | 78049889 | 0 | Xanthomonas | Microbulbifer degradans cellulase |
| 648 | vesicatoria str. 85-10] gi\|78038319\|emb\|CAJ26064.1\| | | | campestris pv. | system protein - SEQ ID 8. |
| | alpha-glucuronidase [Xanthomonas campestris pv. | | | vesicatoria str. 85-10 | |
| | vesicatoria str. 85-10] | | | | |
| 649, | hypothetical protein SNOG_11550 [Phaeosphaeria | 1.11E+08 | 1.00E−106 | Phaeosphaeria | Environmental isolate hydrolase, SEQ |
| 650 | nodorum SN15] | | | nodorum SN15 | ID NO: 44. |
| 651, | hypothetical protein SNOG_08802 [Phaeosphaeria | 1.61E+08 | 0 | Phaeosphaeria | Bacillus clausii alkaline protease |
| 652 | nodorum SN15] | | | nodorum SN15 | coding sequence - SEQ ID 58. |
| 653, | hypothetical protein BACOVA_04385 [Bacteroides | 1.61E+08 | 0 | Bacteroides ovatus | Microbulbifer degradans cellulase |
| 654 | ovatus ATCC 8483] gi\|156108260\|gb\|EDO10005.1\| | | | ATCC 8483 | system protein - SEQ ID 8. |
| | hypothetical protein BACOVA_04385 [Bacteroides | | | | |
| | ovatus ATCC 8483] | | | | |
| 655, | glycoside hydrolase family 43, candidate beta- | 1.5E+08 | 1.00E−148 | Bacteroides vulgatus | Microbulbifer degradans cellulase |
| 656 | xylosidase/alpha-L-arabinofuranosidase [Bacteroides | | | ATCC 8482 | system protein - SEQ ID 8. |
| | vulgatus ATCC 8482] gi\|149931072\|gb\|ABR37770.1\| | | | | |
| | glycoside hydrolase family 43, candidate beta- | | | | |
| | xylosidase/alpha-L-arabinofuranosidase [Bacteroides | | | | |
| | vulgatus AT | | | | |
| 657, | putative esterase [Solibacter usitatus Ellin6076] | 1.17E+08 | 1.00E−108 | Solibacter usitatus | Xylanase from an environmental |
| 658 | gi\|116225263\|gb\|ABJ83972.1\|putative esterase | | | Ellin6076 | sample seq id 14. |
| | [Solibacter usitatus Ellin6076] | | | | |
| 659, | hypothetical protein SNOG_04546 [Phaeosphaeria | 1.11E+08 | 1.00E−124 | Phaeosphaeria | S ambofaciens spiramycin |
| 660 | nodorum SN15] | | | nodorum SN15 | biosynthetic enzyme encoded by |
| | | | | | ORF10*. |
| 661, | alpha-L-arabinofuranosidase [Cochliobolus carbonum]. | 11991219 | 1.00E−149 | Cochliobolus | Xylanase from an environmental |
| 662 | | | | carbonum | sample seq id 14. |
| 663, | hypothetical protein CHGG_00304 [Chaetomium | 1.16E+08 | 1.00E−171 | Chaetomium | C. minitans novel xylanase Cxy1. |
| 664 | globosum CBS 148.51] gi\|88184601\|gb\|EAQ92069.1\| | | | globosum CBS | |
| | hypothetical protein CHGG_00304 [Chaetomium | | | 148.51 | |
| | globosum CBS 148.51] | | | | |
| 665, | cellulase [Cochliobolus carbonum]. | 13346198 | 1.00E−165 | Cochliobolus | Cel45A+Cellobiohydrolase I CBD |
| 666 | | | | carbonum | fusion construct PCR primer SEQ ID |
| | | | | | NO: 16. |
| 667, | hypothetical protein SNOG_15978 [Phaeosphaeria | 1.61E+08 | 0 | Phaeosphaeria | Aspergillus fumigatus Agl1 gene |
| 668 | nodorum SN15] | | | nodorum SN15 | reverse PCR primer, SEQ ID: 17 #1. |
| 669, | glycoside hydrolase, family 3 domain protein [Solibacter | 1.17E+08 | 0 | Solibacter usitatus | Bacteroides fragilis strain 14062 |
| 670 | usitatus Ellin6076] gi\|116224959\|gb\|ABJ83668.1\| | | | Ellin6076 | protein, SEQ: 5227. |
| | glycoside hydrolase, family 3 domain protein [Solibacter | | | | |
| | usitatus Ellin6076] | | | | |
| 671, | Xylan 1,4-beta-xylosidase [Sorangium cellulosum ‘So ce | 1.62E+08 | 0 | Sorangium | Bacillus clausii alkaline protease |
| 672 | 56’] gi\|161163742\|emb\|CAN95047.1\|Xylan 1,4-beta- | | | cellulosum ‘So ce 56 | coding sequence - SEQ ID 58. |
| | xylosidase [Sorangium cellulosum ‘So ce 56’] | | | | |
| 673, | Alpha-L-arabinofuranosidase [Geobacillus | 1.39E+08 | 0 | Geobacillus | Bacillus subtilis abfA gene product. |
| 674 | thermodenitrificans NG80-2] | | | thermodenitrificans | |
| | gi\|134266956\|gb\|ABO67151.1\|Alpha-L- | | | NG80-2 | |
| | arabinofuranosidase [Geobacillus thermodenitrificans | | | | |
| | NG80-2] | | | | |
| 675, | hypothetical protein COPEUT_01466 [Coprococcus | 1.64E+08 | 0 | Coprococcus | Bacterial polypeptide #23667. |
| 676 | eutactus ATCC 27759] gi\|158449501\|gb\|EDP26496.1\| | | | eutactus ATCC | |
| | hypothetical protein COPEUT_01466 [Coprococcus | | | 27759 | |
| | eutactus ATCC 27759] | | | | |
| 677, | Alpha-L-arabinofuranosidase [Caulobacter sp. K31] | 1.14E+08 | 1.00E−179 | Caulobacter sp. K31 | Bacterial polypeptide #23667. |
| 678 | gi\|113729409\|gb\|EAU10485.1\|Alpha-L- | | | | |
| | arabinofuranosidase [Caulobacter sp. K31] | | | | |
| 679, | intra-cellular xylanase [uncultured bacterium] | 31580723 | 1.00E−59 | uncultured bacterium | Xylanase from an environmental |
| 680 | | | | | sample seq id 14. |
| 681, | glycoside hydrolase, family 3 domain protein | 1.5E+08 | 0 | Clostridium | Monterey pine calnexin protein, SEQ |
| 682 | [Clostridium beijerinckii NCIMB 8052] | | | beijerinckii NCIMB | ID: 231. |
| | gi\|149906247\|gb\|ABR37080.1\|glycoside hydrolase, | | | 8052 | |
| | family 3 domain protein [Clostridium beijerinckii NCIMB | | | | |
| | 8052] | | | | |
| 683, | alpha-L-arabinofuranosidase A precursor [Bacteroides | 29345778 | 0 | Bacteroides | Streptomyces sp. arabinofuranosidase |
| 684 | thetaiotaomicron VPI-5482] | | | thetaiotaomicron VPI- | DNA SEQ ID NO: 2. |
| | gi\|29337671\|gb\|AAO75475.1\|alpha-L- | | | 5482 | |
| | arabinofuranosidase A precursor [Bacteroides | | | | |
| | thetaiotaomicron VPI-5482] | | | | |
| 685, | Alpha-L-arabinofuranosidase [Caulobacter sp. K31] | 1.14E+08 | 1.00E−179 | Caulobacter sp. K31 | Bacterial polypeptide #23667. |
| 686 | gi\|113729409\|gb\|EAU10485.1\|Alpha-L- | | | | |
| | arabinofuranosidase [Caulobacter sp. K31] | | | | |
| 687, | hypothetical protein CHGG_05597 [Chaetomium | 1.16E+08 | 1.00E−94 | Chaetomium | Xylanase from an environmental |
| 688 | globosum CBS 148.51] gi\|88181510\|gb\|EAQ88978.1\| | | | globosum CBS | sample seq id 14. |
| | hypothetical protein CHGG_05597 [Chaetomium | | | 148.51 | |
| | globosum CBS 148.51] | | | | |
| 689, | hypothetical protein SNOG_05090 [Phaeosphaeria | 1.11E+08 | 1.00E−169 | Phaeosphaeria | PCR primer for H. insolens Cel6B |
| 690 | nodorum SN15] | | | nodorum SN15 | fungal cellulase coding sequence. |
| 691, | cellulase., Cellulose 1,4-beta-cellobiosidase | 72162575 | 0 | Thermobifida fusca | Bacterial polypeptide #23667. |
| 692 | [Thermobifida fusca YX] | | | YX | |
| | gi\|2506384\|sp\|P26221\|GUN4_THEFU Endoglucanase | | | | |
| | E-4 precursor (Endo-1,4-beta-glucanase E-4) (Cellulase | | | | |
| | E-4) (Cellulase E4) gi\|1817723\|gb\|AAB42155.1\|beta- | | | | |
| | 1,4-endoglucanase precursor [Ther | | | | |
| 693, | beta-glucosidase [Vibrio shilonii AK1] | 1.49E+08 | 1.00E−163 | Vibrio shilonii AK1 | Anti-biofilm polypeptide #100. |
| 694 | gi\|148838481\|gb\|EDL55421.1\|beta-glucosidase [Vibrio | | | | |
| | shilonii AK1] | | | | |
| 695, | hypothetical protein BACOVA_00487 [Bacteroides | 1.61E+08 | 1.00E−147 | Bacteroides ovatus | Microbulbifer degradans cellulase |
| 696 | ovatus ATCC 8483] gi\|156112117\|gb\|EDO13862.1\| | | | ATCC 8483 | system protein - SEQ ID 8. |
| | hypothetical protein BACOVA_00487 [Bacteroides | | | | |
| | ovatus ATCC 8483] | | | | |
| 697, | hypothetical protein BACOVA_00487 [Bacteroides | 1.61E+08 | 1.00E−148 | Bacteroides ovatus | Microbulbifer degradans cellulase |
| 698 | ovatus ATCC 8483] gi\|156112117\|gb\|EDO13862.1\| | | | ATCC 8483 | system protein - SEQ ID 8. |
| | hypothetical protein BACOVA_00487 [Bacteroides | | | | |
| | ovatus ATCC 8483] | | | | |
| 699, | beta-xylosidase [Geobacillus stearothermophilus] | 1.14E+08 | 0 | Geobacillus | Anti-biofilm polypeptide #100. |
| 700 | | | | stearothermophilus | |
| 718, | Endo-1,4-beta-xylanase [Solibacter usitatus Ellin6076] | 1.17E+08 | 1.00E−102 | Solibacter usitatus | Xylanase from an environmental |
| 719 | gi\|116224961\|gb\|ABJ83670.1\|Endo-1,4-beta-xylanase | | | Ellin6076 | sample seq id 14. |
| | [Solibacter usitatus Ellin6076] | | | | |
| 720, | hypothetical protein SNOG_10385 [Phaeosphaeria | 1.61E+08 | 1.00E−141 | Phaeosphaeria | Bacterial polypeptide #23667. |
| 721 | nodorum SN15] | | | nodorum SN15 | |

| | Geneseq | | | Geneseq | | | | | | | |
|---|---|---|---|---|---|---|---|---|---|---|---|
| | Protein | Geneseq | | DNA | | | | | | | |
| SEQ ID | Accession | Protein | | Accession | Geneseq | Query DNA | Query Protein | Geneseq/NR | Gene-seq/NR | Geneseq/NR | Geneseq/NR |
| NO: | Code | Evalue | Geneseq DNA Description | Code | DNA Evalue | Length | Length | DNA Length | Protein Length | % ID Protein | % ID DNA |
| 1, 2 | AAW34989 | 4.00E−89 | Human GPCR protein SEQ ID NO: 68. | ADC87158 | 2.00E−25 | 3450 | 1149 | 0 | 1128 | 25 | |
| 3, 4 | ABR55182 | 1.00E−54 | VSP leader peptide. | ADU48436 | 3.00E−16 | 1356 | 451 | 0 | 456 | 56 | |
| 5, 6 | ABM95926 | 6.00E−52 | Ramoplanin biosynthetic ORF 20 protein. | AAL40781 | 0.016 | 1425 | 474 | 1374 | 457 | 63 | 68 |
| 7, 8 | AEJ60373 | 0 | pHSP-K38 plasmid 2.1 kb insertion encoded | AEA00493 | 4.00E−10 | 2205 | 734 | 0 | 824 | 62 | |
| | | | protein. | | | | | | | | |
| 9, 10 | AAG80266 | 1.00E−129 | Bacillus sp alkaline cellulase PCR primer SEQ | AAI69287 | 3.00E−08 | 2268 | 755 | 2250 | 749 | 87 | 87 |
| | | | ID 22. | | | | | | | | |
| 11, 12 | AAW34989 | 0 | Vibrio harveyi endoglucanase DNA. | AAT94197 | 0 | 3033 | 1010 | 0 | 791 | 44 | |
| 13, 14 | AAB70839 | 1.00E−129 | A. gossypii/S. halstedii fusion construct | AAF61508 | 1.00E−23 | 966 | 321 | 0 | 321 | 67 | |
| | | | containing cellulase DNA. | | | | | | | | |
| 15, 16 | AED12840 | 1.00E−160 | VSP leader peptide. | ADU48437 | 6.00E−42 | 1212 | 403 | 0 | 579 | 83 | |
| 17, 18 | ADN25704 | 0 | VSP leader peptide. | ADU48461 | 4.00E−42 | 2913 | 970 | 0 | 973 | 79 | |
| 19, 20 | AAW95602 | 7.00E−37 | Cancer/angiogenesis/fibrosis-related | ADN38999 | 0.057 | 1299 | 432 | 0 | 469 | 46 | |
| | | | polypeptide, SEQ ID NO: C395. | | | | | | | | |
| 21, 22 | AAR77395 | 0 | Full length Bacillus sp. alkaline cellulase. | AAQ94350 | 8.00E−43 | 2550 | 849 | 0 | 941 | 63 | |
| 23, 24 | ABR55182 | 1.00E−66 | Saccharothrix australiensis endo-beta-1,4- | AAX07410 | 1.00E−11 | 1095 | 364 | 0 | 536 | 51 | |
| | | | glucanase gene. | | | | | | | | |
| 25, 26 | ABR55182 | 2.00E−64 | Nanchangmycin biosynthesis protein NanA9. | ADV99887 | 0.19 | 1098 | 365 | 0 | 329 | 40 | |
| 27, 28 | AAY00865 | 5.00E−72 | Acidothermus cellulolyticus E1 cellulase (E1 | ADA41757 | 3.00E−17 | 600 | 199 | 0 | 529 | 68 | |
| | | | beta-1,4-endoglucanase) DNA. | | | | | | | | |
| 29, 30 | ABJ26902 | 0 | Cellobiohydrolase I activity protein SEQ ID No | ABT23540 | 2.00E−24 | 1605 | 534 | 0 | 529 | 76 | |
| | | | 16. | | | | | | | | |
| 31, 32 | AAY00865 | 0 | Cellobiohydrolase CBH protein fragment. | AAX22095 | 0 | 1611 | 536 | 1611 | 536 | 96 | 92 |
| 33, 34 | AAW57419 | 0 | Cellobiohydrolase I (CBH1) mutant S92T. | ADK81787 | 1.00E−119 | 1515 | 504 | 0 | 505 | 95 | |
| 35, 36 | AAY01076 | 1.00E−102 | Human OPG (osteoprotegerin) K108N protein | ABS54850 | 1.00E−113 | 1350 | 449 | 0 | 394 | 65 | |
| | | | mutant. | | | | | | | | |
| 37, 38 | ADR90316 | 0 | Clostridium josui cellulose degrading cellulase D | ADR90304 | 3.00E−17 | 2226 | 741 | 0 | 741 | 100 | |
| | | | protein. | | | | | | | | |
| 39, 40 | ADR90316 | 0 | VSP leader peptide. | ADU48461 | 4.00E−05 | 3087 | 1028 | 0 | 914 | 59 | |
| 41, 42 | AEF04603 | 3.00E−55 | Novel signal transduction pathway protein, Seq | AAS27844 | 1 | 1485 | 494 | 0 | 499 | 28 | |
| | | | ID 1065. | | | | | | | | |
| 43, 44 | AEF20904 | 1.00E−118 | Rice abiotic stress responsive polypeptide SEQ | ACL28429 | 0.083 | 1854 | 617 | 0 | 1118 | 39 | |
| | | | ID NO: 4152. | | | | | | | | |
| 45, 46 | AEB48738 | 4.00E−48 | SigA2 without bla gene amplifying PCR primer, | AEB45527 | 9.00E−06 | 3006 | 1001 | 3162 | 1053 | 48 | 58 |
| | | | SigA2NotD-P, SEQ ID NO: 52. | | | | | | | | |
| 47, 48 | AEF20904 | 1.00E−118 | Pseudomonas aeruginosa polypeptide #3. | ABD04307 | 0.079 | 1755 | 584 | 489 | 616 | | |
| 49, 50 | AEF04613 | 2.00E−62 | DNA encoding novel human diagnostic protein | AAS73981 | 3.4 | 1251 | 416 | 0 | 499 | 39 | |
| | | | #20574. | | | | | | | | |
| 51, 52 | AEF20904 | 0 | Xylanase from an environmental sample seq id | ADJ35073 | 1.00E−06 | 1740 | 579 | 1761 | 586 | 69 | 72 |
| | | | 14. | | | | | | | | |
| 53, 54 | AAB08774 | 6.00E−74 | Candida essential gene related knockout PCR | ABZ31950 | 9.00E−04 | 1227 | 408 | 0 | 517 | 38 | |
| | | | primer SEQ ID NO 1717. | | | | | | | | |
| 55, 56 | AAB08774 | 5.00E−68 | Sequence of modified xylanase cDNA in clone | AAQ55036 | 5.00E−05 | 1203 | 400 | 1113 | 370 | 41 | 48 |
| | | | pNX-Tac. | | | | | | | | |
| 57, 58 | AEF20904 | 1.00E−120 | Plant transcription factor #1. | ADI42569 | 0.079 | 1755 | 584 | 656 | 616 | | |
| 59, 60 | AED55949 | 2.00E−45 | Maize sugary1 (SU1) exon 8. | AAD42891 | 0.052 | 1179 | 392 | 11779 | 294 | | |
| 61, 62 | AEF20904 | 1.00E−118 | Bacterial polypeptide #10001. | ADS56142 | 0.31 | 1749 | 582 | 0 | 1118 | 41 | |
| 63, 64 | AEF20904 | 1.00E−120 | M. xanthus protein sequence, seq id 9726. | ACL64233 | 1.2 | 1755 | 584 | 4039 | 616 | | |
| 65, 66 | AAE09784 | 2.00E−62 | Serine protease inhibitor gene fragment | AAI67579 | 0.86 | 1245 | 414 | 0 | 499 | 35 | |
| | | | constructing oligo Ab4. | | | | | | | | |
| 67, 68 | AAE09784 | 5.00E−51 | Drosophila melanogaster polypeptide SEQ ID | ABL29670 | 0.18 | 1032 | 343 | 4861 | 395 | | |
| | | | NO 24465. | | | | | | | | |
| 69, 70 | ADR51307 | 7.00E−23 | Equine herpesvirus 4 genome gM deletion | ADP74202 | 0.72 | 1059 | 352 | 0 | 350 | 46 | |
| | | | mutant #1. | | | | | | | | |
| 71, 72 | AAO15063 | 8.00E−44 | Drosophila melanogaster polypeptide SEQ ID | ABL15730 | 0.063 | 1410 | 469 | 10855 | 245 | | |
| | | | NO 24465. | | | | | | | | |
| 73, 74 | AEF04613 | 2.00E−73 | Gene sequence #SEQ ID 1448. | ACC60703 | 0.85 | 1239 | 412 | 2000 | 483 | | |
| 75, 76 | AEF20904 | 1.00E−118 | Rice abiotic stress responsive polypeptide SEQ | ACL34117 | 0.31 | 1755 | 584 | 1047 | 616 | | |
| | | | ID NO: 4152. | | | | | | | | |
| 77, 78 | ABP99336 | 5.00E−28 | Bacterial polypeptide #10001. | ADS58419 | 0.05 | 1140 | 379 | 0 | 492 | 27 | |
| 79, 80 | AEF04613 | 2.00E−63 | Neisseria meningitidis BASB043 gene PCR | AAA49606 | 3.4 | 1251 | 416 | 0 | 499 | 38 | |
| | | | primer lip7-Fm/p. | | | | | | | | |
| 81, 82 | AEB48738 | 4.00E−47 | SigA2 without bla gene amplifying PCR primer, | AEB45527 | 0.002 | 2895 | 964 | 3162 | 1053 | 48 | 59 |
| | | | SigA2NotD-P, SEQ ID NO: 52. | | | | | | | | |
| 83, 84 | AEF04613 | 3.00E−66 | Chemically treated cell signalling DNA | ABL70624 | 0.22 | 1266 | 421 | 6045 | 483 | | |
| | | | sequence#234. | | | | | | | | |
| 85, 86 | AEF20904 | 1.00E−118 | Rice abiotic stress responsive polypeptide SEQ | ACL34117 | 0.31 | 1755 | 584 | 1047 | 616 | | |
| | | | ID NO: 4152. | | | | | | | | |
| 87, 88 | AEF04613 | 7.00E−73 | Human protein encoded by clone | ADB62035 | 0.87 | 1257 | 418 | 2408 | 483 | | |
| | | | ADRGL20047080. | | | | | | | | |
| 89, 90 | AAW56742 | 2.00E−85 | Human prostate expressed polynucleotide SEQ | ABQ88968 | 1.8 | 2484 | 827 | 0 | 814 | 98 | |
| | | | ID NO 803. | | | | | | | | |
| 91, 92 | ADR90316 | 0 | Clostridium josui cellulose degrading cellulase D | ADR90304 | 1.00E−07 | 2274 | 757 | 0 | 741 | 58 | |
| | | | protein. | | | | | | | | |
| 93, 94 | ADR90316 | 0 | Clostridium josui cellulose degrading cellulase D | ADR90304 | 5.00E−07 | 2736 | 911 | 0 | 914 | 65 | |
| | | | protein. | | | | | | | | |
| 95, 96 | ADR90316 | 1.00E−171 | Bacillus licheniformis genomic sequence tag | ABK75466 | 0.009 | 3003 | 1000 | 3276 | 1091 | 67 | 64 |
| | | | (GST) #933. | | | | | | | | |
| 97, 98 | ADR90316 | 1.00E−180 | VSP leader peptide. | ADU48461 | 2.00E−06 | 2091 | 696 | 0 | 854 | 54 | |
| 99, 100 | AED12836 | 0 | VSP leader peptide. | ADU48461 | 0.022 | 1935 | 644 | 0 | 642 | 68 | |
| 101, 102 | AED12836 | 0 | VSP leader peptide. | ADU48461 | 0.022 | 1935 | 644 | 0 | 642 | 68 | |
| 103, 104 | AEH81849 | 4.00E−56 | Pseudomonas aeruginosa polypeptide #3. | ABD11041 | 0.091 | 2010 | 669 | 3084 | 638 | | |
| 105, 106 | AAB08774 | 2.00E−68 | Sequence of modified xylanase cDNA in clone | AAQ55036 | 7.00E−04 | 1005 | 334 | 0 | 517 | 43 | |
| | | | pNX-Tac. | | | | | | | | |
| 107, 108 | AAW44272 | 2.00E−45 | Plant full length insert polynucleotide seqid | ADX53655 | 4.5 | 1647 | 548 | 1776 | 304 | | |
| | | | 4980. | | | | | | | | |
| 109, 110 | AED46544 | 3.00E−32 | Human chemically modified disease associated | ABN80170 | 1 | 1473 | 490 | 1914 | 637 | 17 | 46 |
| | | | gene SEQ ID NO 49. | | | | | | | | |
| 111, 112 | AAB08774 | 4.00E−81 | Rice isoprenoid biosynthesis-associated protein | ADI45632 | 3.6 | 1335 | 444 | 0 | 515 | 40 | |
| | | | #5. | | | | | | | | |
| 113, 114 | AAB08774 | 1.00E−62 | Rice BAC65990.1 protein. | ADV34235 | 0.012 | 1116 | 371 | 1113 | 370 | 39 | 52 |
| 115, 116 | ABP71656 | 3.00E−72 | TokcelR primer used to isolate Tok7B.1 celE | AAD26525 | 5.00E−17 | 939 | 312 | 0 | 1742 | 69 | |
| | | | gene. | | | | | | | | |
| 117, 118 | AEF04603 | 3.00E−68 | Snake venom protease peptide fragment. | ADG83825 | 0.79 | 1152 | 383 | 1407 | 528 | | |
| 119, 120 | AEF04603 | 1.00E−61 | Cryptosporidium hominis protein SEQ ID NO: 2. | AEH38555 | 0.19 | 1104 | 367 | 0 | 499 | 39 | |
| 121, 122 | AAW12381 | 2.00E−92 | Human breast cancer expressed polynucleotide | AAL24695 | 0.71 | 1047 | 348 | 912 | 411 | | |
| | | | 8440. | | | | | | | | |
| 123, 124 | AAW35004 | 1.00E−67 | Novel mar regulated protein (NIMR) #29. | AAS46239 | 4.1 | 1500 | 499 | 0 | 733 | 48 | |
| 125, 126 | AAW35002 | 4.00E−27 | Prokaryotic essential gene #34740. | ACA29992 | 0.73 | 1068 | 355 | 1074 | 357 | 48 | 57 |
| 127, 128 | AAW35002 | 4.00E−28 | Arabidopsis thaliana polynucleotide SEQ ID NO | ABQ65654 | 2.9 | 1068 | 355 | 1074 | 357 | 48 | 56 |
| | | | 197. | | | | | | | | |
| 129, 130 | AED46544 | 7.00E−34 | Cancer-associated protein SEQ ID NO: 19. | AEE04805 | 0.29 | 1641 | 546 | 0 | 365 | 18 | |
| 131, 132 | AAE09784 | 1.00E−62 | Prokaryotic essential gene #34740. | ACA45703 | 3.4 | 1236 | 411 | 0 | 499 | 37 | |
| 133, 134 | AAB08774 | 3.00E−81 | Cow cellulase DNA clones pBKRR 2 and | AEF04597 | 0.28 | 1587 | 528 | 0 | 515 | 36 | |
| | | | pBKRR 16 SEQ ID NO: 3. | | | | | | | | |
| 135, 136 | ADR90316 | 0 | VSP leader peptide. | ADU48458 | 4.00E−07 | 2184 | 727 | 0 | 722 | 58 | |
| 137, 138 | ADN25704 | 0 | A. cellulolyticus Gux1 protein FN_III domain | ABZ76162 | 5.00E−32 | 2916 | 971 | 0 | 973 | 86 | |
| | | | fragment. | | | | | | | | |
| 139, 140 | ADN25704 | 0 | VSP leader peptide. | ADU48461 | 2.00E−28 | 2916 | 971 | 0 | 973 | 81 | |
| 141, 142 | AEF04613 | 5.00E−59 | P. pabuli xyloglucanase XYG1022 DNA | AAD16817 | 0.2 | 1134 | 377 | 0 | 499 | 37 | |
| | | | amplifying PCR primer 189585. | | | | | | | | |
| 143, 144 | AEF04603 | 9.00E−58 | Murine cancer-associated genomic DNA #5. | ADZ13443 | 0.9 | 1308 | 435 | 0 | 499 | 32 | |
| 145, 146 | AEF20904 | 1.00E−119 | Human NURR1-related protein sequence, SEQ | ADB84032 | 4.8 | 1752 | 583 | 0 | 1118 | 42 | |
| | | | ID 79. | | | | | | | | |
| 147, 148 | AAR47496 | 5.00E−81 | Sequence of modified xylanase cDNA in clone | AAQ55036 | 0.019 | 1677 | 558 | 0 | 515 | 34 | |
| | | | pNX-Tac. | | | | | | | | |
| 149, 150 | ADR90317 | 9.00E−76 | Aspergillus fumigatus essential gene protein | ADR84393 | 0.052 | 1188 | 395 | 1113 | 370 | 40 | 51 |
| | | | #10. | | | | | | | | |
| 151, 152 | AEF20904 | 1.00E−119 | M. xanthus protein sequence, seq id 9726. | ACL64233 | 1.2 | 1755 | 584 | 0 | 616 | 42 | |
| 153, 154 | AAB08774 | 1.00E−80 | H. pylori GHPO 1099 gene. | AAX14099 | 0.089 | 1971 | 656 | 0 | 515 | 28 | |
| 155, 156 | AEF20904 | 1.00E−118 | Rice abiotic stress responsive polypeptide SEQ | ACL34117 | 0.32 | 1782 | 593 | 0 | 616 | 40 | |
| | | | ID NO: 4152. | | | | | | | | |
| 157, 158 | AAB08774 | 1.00E−80 | H. pylori GHPO 1099 gene. | AAX14099 | 0.089 | 1971 | 656 | 0 | 515 | 28 | |
| 159, 160 | AEF04613 | 4.00E−69 | Chemically treated cell signalling DNA | ABL70624 | 0.056 | 1266 | 421 | 6045 | 483 | | |
| | | | sequence#234. | | | | | | | | |
| 161, 162 | AAW53973 | 2.00E−49 | Arabidopsis thaliana protein, SEQ ID 1971. | ADA71052 | 0.004 | 1545 | 514 | 2934 | 234 | | |
| 163, 164 | AAR90715 | 1.00E−174 | VSP leader peptide. | ADU48437 | 3.00E−25 | 1476 | 491 | 0 | 579 | 79 | |
| 165, 166 | AAR90715 | 0 | Thermostable cellulase-E3 catalytic domain. | AAT15596 | 5.00E−37 | 1722 | 573 | 0 | 569 | 84 | |
| 167, 168 | AAB08774 | 1.00E−85 | Sequence of modified xylanase cDNA in clone | AAQ55036 | 6.1 | 2199 | 732 | 0 | 515 | 26 | |
| | | | pNX-Tac. | | | | | | | | |
| 169, 170 | ADR90316 | 0 | Clostridium josui cellulose degrading cellulase D | ADR90304 | 2.00E−06 | 3066 | 1021 | 0 | 914 | 61 | |
| | | | protein. | | | | | | | | |
| 171, 172 | ADR90316 | 0 | Clostridium josui cellulose degrading cellulase D | ADR90304 | 7.00E−43 | 2157 | 718 | 0 | 722 | 75 | |
| | | | protein. | | | | | | | | |
| 173, 174 | ADR90316 | 0 | Clostridium josui cellulose degrading cellulase D | ADR90304 | 2.00E−10 | 3009 | 1002 | 0 | 914 | 59 | |
| | | | protein. | | | | | | | | |
| 175, 176 | ADR90316 | 0 | Clostridium josui cellulose degrading cellulase D | ADR90304 | 0.002 | 2646 | 881 | 0 | 914 | 67 | |
| | | | protein. | | | | | | | | |
| 177, 178 | ABP71656 | 0 | A. cellulolyticus Gux1 protein FN_III domain | ABZ76162 | 2.00E−28 | 2589 | 862 | 0 | 1121 | 61 | |
| | | | fragment. | | | | | | | | |
| 179, 180 | ABP71656 | 0 | A. cellulolyticus Gux1 protein FN_III domain | ABZ76162 | 2.00E−11 | 4806 | 1601 | 0 | 973 | 32 | |
| | | | fragment. | | | | | | | | |
| 181, 182 | AED12840 | 1.00E−142 | VSP leader peptide. | ADU48437 | 4.00E−34 | 1455 | 484 | 0 | 569 | 55 | |
| 183, 184 | AAR90715 | 0 | Thermostable cellulase-E3 catalytic domain. | AAT15596 | 7.00E−33 | 1761 | 586 | 0 | 579 | 60 | |
| 185, 186 | AAR90715 | 0 | Thermostable cellulase-E3 catalytic domain. | AAT15596 | 1.00E−37 | 1749 | 582 | 0 | 569 | 65 | |
| 187, 188 | ADR90316 | 0 | Clostridium josui cellulose degrading cellulase D | ADR90304 | 3.00E−11 | 2676 | 891 | 0 | 914 | 59 | |
| | | | protein. | | | | | | | | |
| 189, 190 | ABP71656 | 0 | A. cellulolyticus Gux1 protein FN_III domain | ABZ76162 | 2.00E−11 | 4806 | 1601 | 0 | 973 | 32 | |
| | | | fragment. | | | | | | | | |
| 191, 192 | ABR55182 | 8.00E−67 | Non-reducing saccharide-forming enzyme | AAA10516 | 0.18 | 1035 | 344 | 0 | 382 | 45 | |
| | | | amino acid sequence. | | | | | | | | |
| 193, 194 | ABP71656 | 0 | VSP leader peptide. | ADU48461 | 5.00E−04 | 2700 | 899 | 0 | 854 | 56 | |
| 195, 196 | ADN25704 | 0 | VSP leader peptide. | ADU48461 | 2.00E−31 | 2916 | 971 | 0 | 973 | 84 | |
| 197, 198 | ABR55182 | 3.00E−54 | A. gossypii/S. halstedii fusion construct | AAF61508 | 6.00E−07 | 855 | 284 | 0 | 332 | 46 | |
| | | | containing cellulase DNA. | | | | | | | | |
| 199, 200 | ABR55182 | 5.00E−55 | A. gossypii/S. halstedii fusion construct | AAF61508 | 6.00E−07 | 855 | 284 | 0 | 332 | 46 | |
| | | | containing cellulase DNA. | | | | | | | | |
| 201, 202 | ABM95926 | 3.00E−25 | Mouse stress related vesicle protein, SERP1. | ADP42994 | 4 | 1461 | 486 | 0 | 469 | 49 | |
| 203, 204 | ABM95926 | 3.00E−25 | Mouse stress related vesicle protein, SERP1. | ADP42994 | 4 | 1461 | 486 | 0 | 469 | 49 | |
| 205, 206 | AEF20904 | 1.00E−129 | X campestris umce19A cellulase gene SeqID1. | AEF20903 | 3.00E−04 | 1746 | 581 | 0 | 586 | 43 | |
| 207, 208 | ABP71656 | 0 | VSP leader peptide. | ADU48461 | 1.00E−16 | 2028 | 675 | 0 | 973 | 61 | |
| 209, 210 | AAB70839 | 9.00E−37 | Prokaryotic essential gene #34740. | ACA27085 | 0.014 | 1287 | 428 | 0 | 469 | 54 | |
| 211, 212 | AEF20904 | 1.00E−134 | Microbulbifer degradans cellulase system | AEH81863 | 0.019 | 1674 | 557 | 0 | 616 | 45 | |
| | | | protein - SEQ ID 8. | | | | | | | | |
| 213, 214 | AAB08774 | 6.00E−81 | Human cancer-associated protein HP13-036.1. | ABD32968 | 1.1 | 1587 | 528 | 0 | 515 | 35 | |
| 215, 216 | ABR55182 | 2.00E−70 | Nanchangmycin biosynthesis protein NanA9. | ADV99887 | 5.00E−05 | 1053 | 350 | 0 | 341 | 46 | |
| 217, 218 | ABR55182 | 7.00E−58 | Nanchangmycin biosynthesis protein NanA9. | ADV99887 | 0.003 | 1104 | 367 | 0 | 329 | 37 | |
| 219, 220 | ABR55182 | 3.00E−58 | Nanchangmycin biosynthesis protein NanA9. | ADV99887 | 0.003 | 1104 | 367 | 0 | 329 | 37 | |
| 221, 222 | AAR90715 | 0 | Thermostable cellulase-E3 catalytic domain. | AAT15596 | 7.00E−33 | 1710 | 569 | 0 | 569 | 74 | |
| 223, 224 | AAR90715 | 0 | Thermostable cellulase-E3 catalytic domain. | AAT15596 | 7.00E−33 | 1710 | 569 | 0 | 569 | 74 | |
| 225, 226 | AAR90715 | 0 | Thermostable cellulase-E3 catalytic domain. | AAT15596 | 3.00E−29 | 1725 | 574 | 0 | 569 | 72 | |
| 227, 228 | AAR90715 | 0 | Thermostable cellulase-E3 catalytic domain. | AAT15596 | 7.00E−30 | 1743 | 580 | 0 | 569 | 72 | |
| 229, 230 | AAR90715 | 0 | Thermostable cellulase-E3 catalytic domain. | AAT15596 | 3.00E−29 | 1743 | 580 | 0 | 569 | 72 | |
| 231, 232 | AEF20904 | 1.00E−117 | Xylanase from an environmental sample seq id | ADJ34889 | 0.091 | 2010 | 669 | 2007 | 668 | 72 | 71 |
| | | | 14. | | | | | | | | |
| 233, 234 | AEF04603 | 3.00E−66 | Drosophila melanogaster polypeptide SEQ ID | ABL08153 | 0.86 | 1251 | 416 | 0 | 499 | 35 | |
| | | | NO 24465. | | | | | | | | |
| 235, 236 | ABR55182 | 3.00E−58 | Nanchangmycin biosynthesis protein NanA9. | ADV99887 | 0.003 | 1101 | 366 | 0 | 329 | 38 | |
| 237, 238 | ABR55182 | 3.00E−58 | Nanchangmycin biosynthesis protein NanA9. | ADV99887 | 0.003 | 1101 | 366 | 0 | 329 | 38 | |
| 239, 240 | ABR55182 | 2.00E−58 | Nanchangmycin biosynthesis protein NanA9. | ADV99887 | 0.003 | 1101 | 366 | 0 | 329 | 38 | |
| 241, 242 | ABP71656 | 0 | VSP leader peptide. | ADU48461 | 0.12 | 2550 | 849 | 0 | 854 | 96 | |
| 243, 244 | AAW95602 | 1.00E−29 | Nanchangmycin biosynthesis protein NanA9. | ADV99887 | 7.00E−05 | 1437 | 478 | 0 | 469 | 47 | |
| 245, 246 | AEH81862 | 1.00E−117 | Rice abiotic stress responsive polypeptide SEQ | ACL29091 | 0.079 | 1758 | 585 | 0 | 578 | 41 | |
| | | | ID NO: 4152. | | | | | | | | |
| 247, 248 | AEF20904 | 1.00E−117 | Bacterial polypeptide #10001. | ADS56142 | 0.001 | 1860 | 619 | 2640 | 616 | | |
| 249, 250 | AAB08774 | 2.00E−80 | Rice abiotic stress responsive polypeptide SEQ | ACL26500 | 1.2 | 1677 | 558 | 0 | 515 | 32 | |
| | | | ID NO: 4152. | | | | | | | | |
| 251, 252 | AAW48419 | 2.00E−48 | PCR primer used to amplify an ORF of | AAX91990 | 0.023 | 2016 | 671 | 1230025 | 527 | | |
| | | | Chlamydia pneumoniae. | | | | | | | | |
| 253, 254 | ABP71656 | 0 | A. cellulolyticus Gux1 protein FN_III domain | ABZ76162 | 4.00E−17 | 2529 | 842 | 0 | 854 | 61 | |
| | | | fragment. | | | | | | | | |
| 255, 256 | ABP71656 | 0 | A. cellulolyticus Gux1 protein FN_III domain | ABZ76162 | 2.00E−15 | 2547 | 848 | 0 | 854 | 60 | |
| | | | fragment. | | | | | | | | |
| 257, 258 | ABP71656 | 0 | A. cellulolyticus Gux1 protein FN_III domain | ABZ76162 | 9.00E−15 | 2541 | 846 | 0 | 854 | 60 | |
| | | | fragment. | | | | | | | | |
| 259, 260 | ABP71656 | 0 | A. cellulolyticus Gux1 protein FN_III domain | ABZ76162 | 3.00E−14 | 2535 | 844 | 0 | 854 | 61 | |
| | | | fragment. | | | | | | | | |
| 261, 262 | ADJ35112 | 6.00E−70 | Bacillus subtilis pelA protein sequence SeqID8. | ADO55906 | 8.00E−08 | 1608 | 535 | 1906 | 635 | 47 | 50 |
| 263, 264 | ABP71656 | 0 | A. cellulolyticus Gux1 protein FN_III domain | ABZ76162 | 2.00E−15 | 2523 | 840 | 0 | 854 | 61 | |
| | | | fragment. | | | | | | | | |
| 265, 266 | ABU20587 | 2.00E−06 | Bacteriophage 96 ORF RBS sequence | AAA68609 | 0.63 | 933 | 310 | 0 | 210 | 26 | |
| | | | 96ORF241. | | | | | | | | |
| 267, 268 | ABB80166 | 0 | A. fumigatus AfGOX3. | ABQ80324 | 1.00E−107 | 1422 | 473 | 0 | 468 | 78 | |
| 269, 270 | AED46544 | 3.00E−36 | Oligonucleotide for detecting cytosine | ABQ37581 | 0.18 | 1032 | 343 | 0 | 365 | 29 | |
| | | | methylation SEQ ID NO 20311. | | | | | | | | |
| 271, 272 | AED46544 | 3.00E−35 | Plant full length insert polynucleotide seqid | ADX28493 | 1.2 | 1704 | 567 | 1914 | 637 | 12 | 43 |
| | | | 4980. | | | | | | | | |
| 273, 274 | ADS21197 | 8.00E−57 | Mycobacterium tuberculosis strain H37Rv | AAI99682 | 0.089 | 1977 | 658 | 1368 | 455 | 24 | 33 |
| | | | genome SEQ ID NO 2. | | | | | | | | |
| 275, 276 | AED46544 | 1.00E−33 | Human protein sequence hCP39072. | ACN44892 | 1.2 | 1704 | 567 | 1914 | 637 | 14 | 44 |
| 277, 278 | AED46544 | 9.00E−32 | Plasmid pHM1519 origin of replication fragment | ADO05573 | 1.00E−08 | 921 | 306 | 0 | 365 | 29 | |
| | | | amplifying primer. | | | | | | | | |
| 279, 280 | AED46544 | 4.00E−35 | Human chemically modified disease associated | ABN80170 | 1.2 | 1692 | 563 | 1914 | 637 | 14 | 44 |
| | | | gene SEQ ID NO 49. | | | | | | | | |
| 281, 282 | AEJ12745 | 0 | Cellobiohydrolase II, SEQ ID 2. | ADP84825 | 1.00E−21 | 1407 | 468 | 0 | 458 | 71 | |
| 283, 284 | ADS21197 | 2.00E−55 | Arabidopsis thaliana protein, SEQ ID 1971. | ADA73281 | 1.3 | 1887 | 628 | 1368 | 455 | 24 | 33 |
| 285, 286 | ADS21197 | 2.00E−75 | Type II diabetes gene SEQ ID NO 7. | ADT77142 | 0.69 | 1020 | 339 | 0 | 1302 | 46 | |
| 287, 288 | AAU79549 | 1.00E−126 | Bacterial polypeptide #10001. | ADS63386 | 0.008 | 2823 | 940 | 0 | 2312 | 50 | |
| 289, 290 | AED46544 | 8.00E−36 | Prokaryotic essential gene #34740. | ACA52811 | 1.2 | 1704 | 567 | 1914 | 637 | 14 | 43 |
| 291, 292 | AEH81862 | 1.00E−85 | Maize carbon assimilation pathway enzyme | ADP59233 | 0.29 | 1638 | 545 | 0 | 547 | 37 | |
| | | | cDNA #19. | | | | | | | | |
| 293, 294 | AEF20904 | 5.00E−89 | Human cDNA clone (3′-primer) SEQ ID | AAH17050 | 0.073 | 1635 | 544 | 0 | 547 | 38 | |
| | | | NO: 5589. | | | | | | | | |
| 295, 296 | AEF20904 | 1.00E−127 | Microbulbifer degradans cellulase system | AEH81863 | 0.33 | 1857 | 618 | 2007 | 668 | 42 | 51 |
| | | | protein - SEQ ID 8. | | | | | | | | |
| 297, 298 | AEF20904 | 1.00E−131 | X campestris umce19A cellulase gene SeqID1. | AEF20903 | 3.00E−04 | 1722 | 573 | 1761 | 586 | 44 | 50 |
| 299, 300 | AEF20904 | 1.00E−130 | Microbulbifer degradans cellulase system | AEH81863 | 8.00E−05 | 1746 | 581 | 1761 | 586 | 44 | 50 |
| | | | protein - SEQ ID 8. | | | | | | | | |
| 301, 302 | AEF20904 | 1.00E−130 | X campestris umce19A cellulase gene SeqID1. | AEF20903 | 3.00E−04 | 1743 | 580 | 0 | 586 | 44 | |
| 303, 304 | AEF20904 | 1.00E−130 | Microbulbifer degradans cellulase system | AEH81863 | 0.31 | 1737 | 578 | 2007 | 668 | 45 | 54 |
| | | | protein - SEQ ID 8. | | | | | | | | |
| 305, 306 | AEH81835 | 3.00E−62 | Drosophila melanogaster polypeptide SEQ ID | ABL10402 | 0.29 | 1623 | 540 | 16962 | 1167 | | |
| | | | NO 24465. | | | | | | | | |
| 307, 308 | AEH81835 | 3.00E−63 | Soybean polymorphic locus, SEQ ID 6. | AEI27639 | 0.073 | 1641 | 546 | 1439 | 1167 | | |
| 309, 310 | AEF20904 | 9.00E−80 | Xylanase from an environmental sample seq id | ADJ35073 | 0.074 | 1647 | 548 | 0 | 547 | 35 | |
| | | | 14. | | | | | | | | |
| 311, 312 | AEH81835 | 1.00E−68 | Microbulbifer degradans cellulase system | AEH81836 | 0.018 | 1569 | 522 | 3504 | 1167 | | |
| | | | protein - SEQ ID 8. | | | | | | | | |
| 313, 314 | AAW48419 | 7.00E−37 | Drosophila melanogaster polypeptide SEQ ID | ABL12476 | 0.92 | 1332 | 443 | 4154 | 527 | | |
| | | | NO 24465. | | | | | | | | |
| 315, 316 | AAR90715 | 0 | Thermostable cellulase-E3 catalytic domain. | AAT15595 | 2.00E−30 | 1734 | 577 | 0 | 579 | 95 | |
| 317, 318 | ADC73058 | 3.00E−89 | Trametes hirsuta cellulolytic enzyme-related | ADC73057 | 6.00E−05 | 1281 | 426 | 1704 | 453 | | |
| | | | protein - SEQ ID 12. | | | | | | | | |
| 319, 320 | ADC73058 | 2.00E−89 | Trametes hirsuta cellulolytic enzyme-related | ADC73057 | 4.00E−06 | 1281 | 426 | 1704 | 453 | | |
| | | | protein - SEQ ID 12. | | | | | | | | |
| 321, 322 | ADC73058 | 2.00E−89 | Trametes hirsuta cellulolytic enzyme-related | ADC73057 | 6.00E−05 | 1281 | 426 | 1704 | 453 | | |
| | | | protein - SEQ ID 12. | | | | | | | | |
| 323, 324 | ABM95926 | 3.00E−82 | A. gossypii/S. halstedii fusion construct | AAF61508 | 5.00E−11 | 984 | 327 | 0 | 500 | 53 | |
| | | | containing cellulase DNA. | | | | | | | | |
| 325, 326 | ABM95926 | 2.00E−83 | A. gossypii/S. halstedii fusion construct | AAF61508 | 2.00E−13 | 984 | 327 | 0 | 500 | 53 | |
| | | | containing cellulase DNA. | | | | | | | | |
| 327, 328 | ABM95926 | 3.00E−82 | A. gossypii/S. halstedii fusion construct | AAF61508 | 5.00E−11 | 984 | 327 | 0 | 500 | 53 | |
| | | | containing cellulase DNA. | | | | | | | | |
| 329, 330 | ABM95926 | 1.00E−82 | A. gossypii/S. halstedii fusion construct | AAF61508 | 2.00E−13 | 984 | 327 | 0 | 500 | 53 | |
| | | | containing cellulase DNA. | | | | | | | | |
| 331, 332 | ABM95926 | 1.00E−82 | A. gossypii/S. halstedii fusion construct | AAF61508 | 5.00E−11 | 984 | 327 | 0 | 500 | 53 | |
| | | | containing cellulase DNA. | | | | | | | | |
| 333, 334 | ADC73058 | 6.00E−89 | Trametes hirsuta cellulolytic enzyme-related | ADC73057 | 6.00E−05 | 1281 | 426 | 1704 | 453 | | |
| | | | protein - SEQ ID 12. | | | | | | | | |
| 335, 336 | ADC73058 | 3.00E−89 | Trametes hirsuta cellulolytic enzyme-related | ADC73057 | 6.00E−05 | 1281 | 426 | 1704 | 453 | | |
| | | | protein - SEQ ID 12. | | | | | | | | |
| 337, 338 | AEF20904 | 1.00E−134 | Microbulbifer degradans cellulase system | AEH81863 | 0.021 | 1818 | 605 | 0 | 616 | 42 | |
| | | | protein - SEQ ID 8. | | | | | | | | |
| 339, 340 | AEF20904 | 4.00E−21 | Human cancer associated sequence HP1-10- | ADQ97275 | 1.2 | 1674 | 557 | 0 | 570 | 36 | |
| | | | 003, SEQ ID 12. | | | | | | | | |
| 341, 342 | ABR55182 | 2.00E−46 | M. xanthus protein sequence, seq id 9726. | ACL64337 | 0.003 | 1239 | 412 | 1422 | 473 | 53 | 65 |
| 343, 344 | ABR55182 | 3.00E−46 | M. xanthus protein sequence, seq id 9726. | ACL64337 | 0.003 | 1239 | 412 | 1422 | 473 | 53 | 66 |
| 345, 346 | ABP73029 | 1.00E−136 | Acremonium cellulolyticus cellulase encoding | AAT91640 | 0.017 | 1533 | 510 | 0 | 1128 | 54 | |
| | | | DNA. | | | | | | | | |
| 347, 348 | AAW48419 | 2.00E−57 | Human protein useful for treating neurological | ADR08112 | 3.3 | 1197 | 398 | 2923 | 527 | | |
| | | | disease Seq 1966. | | | | | | | | |
| 349, 350 | AEH81858 | 1.00E−105 | Vibrio harveyi endoglucanase DNA. | AAT94197 | 1.00E−04 | 2460 | 819 | 0 | 1128 | 37 | |
| 351, 352 | AEH81858 | 0 | CAPON-2 amino acid sequence. | ABA97202 | 0.096 | 2118 | 705 | 0 | 791 | 60 | |
| 353, 354 | AAW95602 | 5.00E−65 | Hyperthermophile Methanopyrus kandleri | ADM27081 | 0.96 | 1383 | 460 | 1694968 | 490 | | |
| | | | protein #28. | | | | | | | | |
| 355, 356 | ABJ26888 | 0 | Cellobiohydrolase I activity protein SEQ ID No | ABT23540 | 7.00E−45 | 1359 | 452 | 0 | 452 | 76 | |
| | | | 16. | | | | | | | | |
| 357, 358 | AEJ12745 | 0 | Glucose isomerase SEQ ID NO 20. | AED46539 | 3.00E−87 | 1419 | 472 | 0 | 471 | 89 | |
| 359, 360 | AAB81926 | 0 | Acremonium cellulolyticus xylanase precursor. | AAF85588 | 0 | 1590 | 529 | 0 | 529 | 95 | |
| 361, 362 | AAE16324 | 0 | VSP leader peptide. | ADU48458 | 4.00E−07 | 2220 | 739 | 0 | 739 | 100 | |
| 363, 364 | AEE20076 | 0 | Bacillus licheniformis genomic sequence tag | ABK73355 | 0.065 | 1455 | 484 | 0 | 484 | 100 | |
| | | | (GST) #933. | | | | | | | | |
| 365, 366 | ADR90315 | 1.00E−144 | VSP leader peptide. | ADU48455 | 3.00E−04 | 1401 | 466 | 0 | 477 | 99 | |
| 367, 368 | AEF20904 | 1.00E−119 | M. xanthus protein sequence, seq id 9726. | ACL64233 | 1.2 | 1755 | 584 | 0 | 616 | 42 | |
| 431, 432 | ADJ34940 | 0 | Xylanase from an environmental sample seq id | ADJ34939 | 0 | 1836 | 611 | 1836 | 611 | | |
| | | | 14. | | | | | | | | |
| 433, 434 | ADJ34826 | 0 | Xylanase from an environmental sample seq id | ADJ34825 | 0 | 1893 | 630 | 0 | 592 | 57 | |
| | | | 14. | | | | | | | | |
| 435, 436 | AAB99272 | 3.00E−54 | Human gene NM_022875, SEQ ID NO 12308. | ADE62144 | 2.1 | 2997 | 998 | 3966 | 1321 | 35 | 52 |
| 437, 438 | AED34890 | 1.00E−103 | Endoglucanase encoded by endo3 gene. | AAQ13001 | 1.00E−112 | 1353 | 450 | 2977 | 584 | | |
| 439, 440 | ADJ35128 | 0 | Xylanase from an environmental sample seq id | ADJ35127 | 0 | 2217 | 738 | 2217 | 738 | | |
| | | | 14. | | | | | | | | |
| 441, 442 | ADJ35146 | 0 | Xylanase from an environmental sample seq id | ADJ35145 | 0 | 5043 | 1680 | 5040 | 1680 | | |
| | | | 14. | | | | | | | | |
| 443, 444 | ADJ34914 | 0 | Xylanase from an environmental sample seq id | ADJ34913 | 0 | 2823 | 940 | 0 | 1077 | 99 | |
| | | | 14. | | | | | | | | |
| 445, 446 | AAW34987 | 0 | Vibrio harveyi endoglucanase DNA. | AAT94195 | 0 | 2628 | 875 | 0 | 884 | 45 | |
| 447, 448 | AAE16325 | 0 | TokcelR primer used to isolate Tok7B.1 celE | AAD26525 | 1.00E−104 | 2724 | 907 | 3003 | 1000 | 74 | 75 |
| | | | gene. | | | | | | | | |
| 449, 450 | ADS14829 | 2.00E−28 | Plant full length insert polynucleotide seqid | ADO84476 | 0.048 | 1089 | 362 | 1077 | 358 | 83 | 83 |
| | | | 4980. | | | | | | | | |
| 451, 452 | AEH62812 | 1.00E−131 | Plant full length insert polynucleotide seqid | ADX53508 | 2.00E−08 | 1671 | 556 | 0 | 492 | 51 | |
| | | | 4980. | | | | | | | | |
| 453, 454 | ADJ34940 | 1.00E−11 | DNA encoding a polyphenol oxidase F | AAA63731 | 0.26 | 1503 | 500 | 0 | 2636 | 26 | |
| | | | polypeptide. | | | | | | | | |
| 455, 456 | ADN25642 | 3.00E−11 | Plant polypeptide, SEQ ID 5546. | ADT19227 | 2 | 774 | 257 | 0 | 291 | 100 | |
| 457, 458 | ADS14829 | 2.00E−41 | M. xanthus protein sequence, seq id 9726. | ACL64540 | 7.00E−14 | 1311 | 436 | 0 | 438 | 100 | |
| 459, 460 | AEH62893 | 3.00E−39 | F. rubripes erythrocyte differentiation factor, | ADO05609 | 0.38 | 594 | 197 | 0 | 201 | 71 | |
| | | | Codanin-1. | | | | | | | | |
| 461, 462 | AAW29456 | 3.00E−65 | Maltogenic alpha-amylase signal peptide PCR | AAT29043 | 0.018 | 1572 | 523 | 1929 | 642 | 95 | 95 |
| | | | primer DK16. | | | | | | | | |
| 471, 472 | ADS14829 | 4.00E−27 | Human protein sequence hCP39072. | ACN44350 | 0.75 | 1095 | 364 | 0 | 364 | 100 | |
| 489, 490 | AKT18586 | 1.00E−144 | Bacterial polypeptide #23667. | ADS48454 | 6.00E−14 | 1146 | 381 | 0 | 382 | 69 | |
| 491, 492 | AAW46814 | 0 | Endo beta-1,4-gluconase peptide 3. | AAV16436 | 0 | 999 | 332 | 999 | 332 | 99 | 97 |
| 493, 494 | AAW46814 | 0 | Endo beta-1,4-gluconase peptide 3. | AAV16436 | 0 | 999 | 332 | 999 | 332 | 99 | 97 |
| 495, 496 | AAW15563 | 1.00E−112 | Talaromyces emersonii beta-glucanase CEC | AAD20928 | 2.00E−07 | 999 | 332 | 0 | 335 | | |
| | | | protein. | | | | | | | | |
| 497, 498 | ADN20544 | 1.00E−154 | Endoglucanase (60 kDa Family 5 cellulase) | AAT29035 | 6.00E−45 | 1200 | 399 | 0 | 382 | 65 | |
| | | | cDNA sequence. | | | | | | | | |
| 499, 500 | AKT18586 | 1.00E−145 | Bacterial polypeptide #23667. | ADS48454 | 6.00E−11 | 1149 | 382 | 0 | 382 | 84 | |
| 501, 502 | ADN20544 | 1.00E−155 | P. brasilianum cel5c endoglucanase reverse | AKT18585 | 9.00E−35 | 1200 | 399 | 0 | 390 | 66 | |
| | | | PCR primer, SEQ ID NO: 15. | | | | | | | | |
| 503, 504 | ADC58031 | 1.00E−114 | Talaromyces emersonii beta-glucanase CEC | AAD20928 | 1.00E−08 | 993 | 330 | 0 | 337 | 64 | |
| | | | protein. | | | | | | | | |
| 505, 506 | AEB00295 | 1.00E−178 | P. brasilianum cel5c endoglucanase reverse | AKT18585 | 6.00E−79 | 1230 | 409 | 0 | 384 | 72 | |
| | | | PCR primer, SEQ ID NO: 15. | | | | | | | | |
| 507, 508 | AAE12786 | 1.00E−124 | Talaromyces emersonii beta-glucanase CEC | AAD20928 | 4.00E−15 | 1023 | 340 | 0 | 1272 | 67 | |
| | | | protein. | | | | | | | | |
| 509, 510 | AEB00295 | 0 | P. brasilianum cel5c endoglucanase reverse | AKT18585 | 1.00E−120 | 1224 | 407 | 0 | 388 | 75 | |
| | | | PCR primer, SEQ ID NO: 15. | | | | | | | | |
| 511, 512 | AKT18592 | 1.00E−163 | Bacterial polypeptide #23667. | ADS60941 | 7.00E−17 | 1233 | 410 | 0 | 410 | 95 | |
| 513, 514 | AKT18586 | 1.00E−145 | Bacterial polypeptide #23667. | ADS60941 | 4.00E−12 | 1149 | 382 | 0 | 382 | 84 | |
| 515, 516 | AAW56742 | 1.00E−85 | Human prostate expressed polynucleotide SEQ | ABQ88968 | 1 | 1368 | 455 | 0 | 814 | 100 | |
| | | | ID NO 803. | | | | | | | | |
| 517, 518 | AKT18592 | 1.00E−163 | Bacterial polypeptide #23667. | ADS60941 | 7.00E−17 | 1233 | 410 | 0 | 410 | 95 | |
| 519, 520 | AKT18592 | 0 | P. brasilianum cel5c endoglucanase reverse | AKT18591 | 1.00E−109 | 1260 | 419 | 1488 | 421 | | |
| | | | PCR primer, SEQ ID NO: 15. | | | | | | | | |
| 521, 522 | ABB80166 | 0 | Glucose isomerase SEQ ID NO 20. | AED46552 | 6.00E−61 | 1413 | 470 | 0 | 459 | 86 | |
| 523, 524 | ABJ26885 | 0 | Cellobiohydrolase I activity protein SEQ ID No | ABT23507 | 7.00E−70 | 1569 | 522 | 0 | 523 | | |
| | | | 16. | | | | | | | | |
| 525, 526 | AEH81867 | 0 | H. salinarum nucleoside diphosphate kinase, | AEK17721 | 0.12 | 2481 | 826 | 0 | 856 | 58 | |
| | | | SEQ ID NO: 4. | | | | | | | | |
| 527, 528 | ADC51490 | 1.00E−180 | Cryptosporidium hominis protein SEQ ID NO: 2. | AEH40716 | 1.3 | 1725 | 574 | 1431 | 571 | | |
| 529, 530 | AAE23633 | 0 | Thermoanaerobacter cellulolyticus thermostable | AAV23285 | 7.00E−05 | 1347 | 448 | 0 | 448 | 100 | |
| | | | beta-glucosidase. | | | | | | | | |
| 531, 532 | ADS30418 | 1.00E−152 | Tib10 beta-gly, SEQ ID 10. | ADQ75574 | 7.00E−05 | 1362 | 453 | 0 | 463 | | |
| 533, 534 | ADR51303 | 1.00E−117 | Human Klotho cDNA, SEQ ID NO: 5. | AAH23959 | 0.066 | 1338 | 445 | 0 | 456 | | |
| 535, 536 | ADR51299 | 0 | Anti-biofilm polypeptide #100. | ADR51298 | 0 | 1263 | 420 | 1272 | 423 | 81 | 72 |
| 537, 538 | ADR51283 | 0 | Thermococcus 9N2-31B/G glycosidase gene | AAV36911 | 0 | 2166 | 721 | 2166 | 721 | 99 | 99 |
| | | | coding region. | | | | | | | | |
| 539, 540 | ADR51303 | 0 | Anti-biofilm polypeptide #100. | ADR51302 | 0 | 1389 | 462 | 0 | 459 | | |
| 541, 542 | ADR51303 | 1.00E−110 | Bacterial polypeptide #23667. | ADS56264 | 3.00E−04 | 1350 | 449 | 0 | 454 | | |
| 543, 544 | ADN26272 | 1.00E−125 | Bacterial polypeptide #23667. | ADS56139 | 6.00E−05 | 1188 | 395 | 0 | 411 | | |
| 545, 546 | ADN01220 | 1.00E−123 | T. bispora NRRL 15568 beta-glucosidase. | ADN01219 | 7.00E−05 | 1386 | 461 | 0 | 467 | | |
| 547, 548 | ADS30418 | 1.00E−180 | Bacterial polypeptide #23667. | ADS56264 | 8.00E−11 | 1377 | 458 | 0 | 463 | | |
| 549, 550 | ADR51303 | 1.00E−141 | Streptococcus sp. H021 Orf2, oxidoreductase. | AAD47222 | 1.1 | 1404 | 467 | 0 | 471 | 56 | |
| 551, 552 | ADS21519 | 1.00E−119 | Anti-biofilm polypeptide #100. | ADR51312 | 2.00E−08 | 1230 | 409 | 0 | 439 | 53 | |
| 553, 554 | ADZ83372 | 5.00E−78 | Anti-biofilm polypeptide #100. | ADR51312 | 0.25 | 1284 | 427 | 1314 | 423 | | |
| 555, 556 | AAR88093 | 0 | Thermostable beta-galactosidase conserved | AAT09293 | 0 | 1419 | 472 | 1419 | 472 | 100 | 100 |
| | | | sequence (Box 10). | | | | | | | | |
| 557, 558 | AAR25384 | 0 | PCR primer for cDNA encoding a beta- | AAA63953 | 3.00E−05 | 2160 | 719 | 0 | 696 | | |
| | | | glucosidase polypeptide. | | | | | | | | |
| 559, 560 | ADR51229 | 0 | Anti-biofilm polypeptide #100. | ADR51228 | 0 | 1431 | 476 | 0 | 467 | | |
| 561, 562 | ADN01220 | 1.00E−107 | Bacterial polypeptide #23667. | ADT43152 | 4.00E−06 | 1350 | 449 | 0 | 440 | 65 | |
| 563, 564 | ADR51303 | 0 | Anti-biofilm polypeptide #100. | ADR51302 | 1.00E−131 | 1389 | 462 | 0 | 459 | | |
| 565, 566 | ADS30418 | 1.00E−143 | Human myocardial infarction-associated gene | ADQ38981 | 2.00E−05 | 1347 | 448 | 0 | 448 | 57 | |
| | | | derived protein, SEQ ID 835. | | | | | | | | |
| 567, 568 | ABU24282 | 5.00E−34 | S. epidermidis genomic polynucleotide | AAH54621 | 0.08 | 1620 | 539 | 0 | 520 | 23 | |
| | | | sequence SEQ ID NO: 4137. | | | | | | | | |
| 569, 570 | ADN01220 | 1.00E−107 | Protein encoded by Prokaryotic essential gene | ACA25213 | 0.017 | 1350 | 449 | 0 | 440 | 67 | |
| | | | #30232. | | | | | | | | |
| 571, 572 | ADR51303 | 1.00E−140 | Bacterial polypeptide #23667. | ADS56139 | 0.27 | 1404 | 467 | 0 | 471 | 56 | |
| 573, 574 | AEX28563 | 1.00E−118 | Arabidopsis herbicide target gene 4036 cDNA. | AAA50081 | 1.9 | 2457 | 818 | 0 | 831 | 41 | |
| 575, 576 | AAE23633 | 1.00E−133 | Plant full length insert polynucleotide seqid | ADX11847 | 0.001 | 1362 | 453 | 0 | 446 | 56 | |
| | | | 4980. | | | | | | | | |
| 577, 578 | ABU48326 | 1.00E−111 | Arabidopsis thaliana polynucleotide SEQ ID NO | ABQ65793 | 0.44 | 2229 | 742 | 0 | 766 | 56 | |
| | | | 197. | | | | | | | | |
| 579, 580 | ADN01220 | 1.00E−156 | Bacterial polypeptide #23667. | ADS56264 | 6.00E−30 | 1434 | 477 | 0 | 478 | 82 | |
| 581, 582 | ADH88405 | 1.00E−178 | Listeria innocua DNA sequence #303. | ABQ70760 | 0.007 | 2268 | 755 | 0 | 743 | | |
| 583, 584 | AEH81871 | 0 | Vibrio harveyi endoglucanase DNA. | AAT94214 | 2.00E−06 | 2577 | 858 | 0 | 888 | 64 | |
| 585, 586 | AEX29253 | 1.00E−112 | Vibrio harveyi endoglucanase DNA. | AAT94214 | 0.007 | 2331 | 776 | 0 | 766 | 47 | |
| 587, 588 | AAR97199 | 0 | Chimaeric thermostable beta-glucosidase. | AAT32999 | 4.00E−26 | 2238 | 745 | 0 | 748 | 59 | |
| 589, 590 | ADR51313 | 0 | Anti-biofilm polypeptide #100. | ADR51312 | 0 | 1314 | 437 | 1314 | 437 | | |
| 591, 592 | AAE23633 | 1.00E−109 | P. chrysosporium CKG4 lignin peroxidase | ABK86730 | 3.00E−10 | 1455 | 484 | 0 | 451 | 42 | |
| | | | (ligninase)(LIP). | | | | | | | | |
| 593, 594 | ADC51488 | 8.00E−79 | DNA sequence of Myxococcus fulvus | AAA75307 | 8.00E−18 | 2007 | 668 | 0 | 671 | 66 | |
| | | | pyrrolnitrin gene region. | | | | | | | | |
| 595, 596 | AEX29253 | 1.00E−155 | Protein encoded by Prokaryotic essential gene | ACA45681 | 0.007 | 2244 | 747 | 0 | 763 | | |
| | | | #30232. | | | | | | | | |
| 597, 598 | AEJ12745 | 0 | Cellobiohydrolase CBH II protein. | AAN50359 | 0 | 1416 | 471 | 0 | 471 | 100 | |
| 599, 600 | AAW57419 | 0 | Cellobiohydrolase I (CBH1) mutant S92T. | ADK81787 | 1.00E−141 | 1518 | 505 | 0 | 505 | 100 | |
| 601, 602 | ABB80166 | 0 | Glucose isomerase SEQ ID NO 20. | AED46552 | 6.00E−61 | 1413 | 470 | 0 | 459 | 86 | |
| 603, 604 | ABJ26885 | 0 | Cellobiohydrolase I activity protein SEQ ID No | ABT23507 | 7.00E−70 | 1569 | 522 | 0 | 523 | | |
| | | | 16. | | | | | | | | |
| 605, 606 | ABJ26902 | 0 | Cellobiohydrolase I activity protein SEQ ID No | ABT23540 | 1.00E−90 | 1638 | 545 | 0 | 537 | 78 | |
| | | | 16. | | | | | | | | |
| 607, 608 | ABJ26901 | 0 | Cellobiohydrolase I activity protein SEQ ID No | ABT23510 | 2.00E−54 | 1338 | 445 | 0 | 448 | 71 | |
| | | | 16. | | | | | | | | |
| 609, 610 | AAW95029 | 0 | Cellobiohydrolase I activity protein SEQ ID No | ABT23506 | 4.00E−28 | 1365 | 454 | 0 | 455 | 93 | |
| | | | 16. | | | | | | | | |
| 611, 612 | ADW12302 | 8.00E−45 | Endoplasmic reticulum retaining peptide. | AAC84644 | 0.056 | 1158 | 385 | 0 | 400 | 53 | |
| 613, 614 | AED46513 | 1.00E−103 | Plasmid pNOV4031 amylase fusion amino acid | ACC44578 | 0.002 | 1995 | 664 | 960 | 319 | 27 | 28 |
| | | | sequence SEQ ID NO: 16. | | | | | | | | |
| 615, 616 | AAR15237 | 0 | Linking B region #8 derived from a | AAQ14838 | 0 | 1545 | 514 | 0 | 514 | 97 | |
| | | | (hemi)cellulose-degrading enzyme. | | | | | | | | |
| 617, 618 | ABJ26902 | 0 | Cellobiohydrolase I activity protein SEQ ID No | ABT23540 | 2.00E−20 | 1581 | 526 | 0 | 521 | 67 | |
| | | | 16. | | | | | | | | |
| 619, 620 | ABB80166 | 0 | A. fumigatus AfGOX3. | ABQ80324 | 3.00E−93 | 1395 | 464 | 0 | 454 | 77 | |
| 621, 622 | AAW35004 | 1.00E−157 | Protein encoded by Prokaryotic essential gene | ACA45681 | 0.008 | 2412 | 803 | 2421 | 806 | 67 | 71 |
| | | | #30232. | | | | | | | | |
| 623, 624 | AEH47476 | 0 | Environmental isolate hydrolase, SEQ ID | AEH47475 | 0 | 1293 | 430 | 1293 | 430 | | |
| | | | NO: 44. | | | | | | | | |
| 625, 626 | AAW35004 | 1.00E−173 | Protein encoded by Prokaryotic essential gene | ACA45681 | 0.008 | 2358 | 785 | 0 | 765 | 53 | |
| | | | #30232. | | | | | | | | |
| 627, 628 | AEC74753 | 5.00E−85 | Myceliophthora thermophila xylanase cDNA. | AAT74074 | 2.00E−13 | 1002 | 333 | 0 | 384 | 65 | |
| 629, 630 | AEL86665 | 4.00E−97 | Monterey pine calnexin protein, SEQ ID: 231. | AGI25306 | 8.00E−08 | 1524 | 507 | 1281 | 426 | 55 | 56 |
| 631, 632 | AAY52699 | 1.00E−154 | Aspergillus fumigatus essential gene protein | ADR84318 | 0.44 | 2232 | 743 | 0 | 755 | | |
| | | | #385. | | | | | | | | |
| 633, 634 | AAR25384 | 0 | PCR primer for cDNA encoding a beta- | AAA63953 | 3.00E−05 | 2160 | 719 | 0 | 696 | | |
| | | | glucosidase polypeptide. | | | | | | | | |
| 635, 636 | AEH81913 | 2.00E−70 | Angiotensin gene methylation analysing | AAD28365 | 2.9 | 981 | 326 | 987 | 328 | 92 | 93 |
| | | | oligonucleotide #2. | | | | | | | | |
| 637, 638 | ADZ51810 | 1.00E−179 | Plant cDNA #31. | ADJ40527 | 0.34 | 1734 | 577 | 0 | 559 | | |
| 639, 640 | AEH47790 | 0 | Environmental isolate hydrolase, SEQ ID | AEH47789 | 0 | 1581 | 526 | 1581 | 526 | | |
| | | | NO: 44. | | | | | | | | |
| 641, 642 | AEH47208 | 0 | Environmental isolate hydrolase, SEQ ID | AEH47207 | 0 | 2040 | 679 | 2040 | 679 | | |
| | | | NO: 44. | | | | | | | | |
| 643, 644 | AEH47654 | 0 | Environmental isolate hydrolase, SEQ ID | AEH47653 | 0 | 1623 | 540 | 1623 | 540 | | |
| | | | NO: 44. | | | | | | | | |
| 645, 646 | ADS30418 | 1.00E−146 | Mouse protein tyrosine phosphatase | AAT85389 | 4.1 | 1362 | 453 | 0 | 463 | | |
| | | | PTPepsilon. | | | | | | | | |
| 647, 648 | AEH81915 | 0 | Bacterial polypeptide #23667. | ADT46252 | 0.007 | 2163 | 720 | 0 | 778 | 62 | |
| 649, 650 | AEH47274 | 1.00E−149 | Environmental isolate hydrolase, SEQ ID | AEH47273 | 0 | 759 | 252 | 759 | 252 | | |
| | | | NO: 44. | | | | | | | | |
| 651, 652 | AEG60866 | 1.00E−127 | N-terminal peptide of the alpha-glucuronidase | AAV05187 | 0.13 | 2508 | 835 | 0 | 836 | | |
| | | | protein. | | | | | | | | |
| 653, 654 | AEH81915 | 0 | Microbulbifer degradans cellulase system | AEH81916 | 0.002 | 2046 | 681 | 0 | 711 | | |
| | | | protein - SEQ ID 8. | | | | | | | | |
| 655, 656 | AEH81913 | 1.00E−117 | B. amyloliquefaciens bacillomycin A protein Seq | ADW21121 | 0.012 | 966 | 321 | 0 | 323 | | |
| | | | 3. | | | | | | | | |
| 657, 658 | ADJ35150 | 0 | Xylanase from an environmental sample seq id | ADJ35149 | 0 | 3246 | 1081 | 3246 | 1081 | | |
| | | | 14. | | | | | | | | |
| 659, 660 | ADN97699 | 4.00E−50 | Bacterial polypeptide #23667. | ADS58668 | 0.013 | 1026 | 341 | 0 | 346 | 64 | |
| 661, 662 | ADJ34838 | 1.00E−112 | S ambofaciens spiramycin biosynthetic enzyme | ADN97710 | 2.00E−34 | 831 | 276 | 978 | 325 | 92 | 95 |
| | | | encoded by ORF10*. | | | | | | | | |
| 663, 664 | AAB29041 | 1.00E−180 | Partial Chrysoporium GPD1. | AAI72046 | 6.00E−88 | 1116 | 371 | 3028 | 384 | | |
| 665, 666 | AEM25422 | 1.00E−124 | Melanocarpus albomyces 20 K cellulase | AEL87188 | 2.00E−11 | 1182 | 393 | 0 | 423 | 67 | |
| | | | protein. | | | | | | | | |
| 667, 668 | AEF10657 | 0 | F. venenatum alpha-glucosidase DNA | AEF93568 | 1.00E−14 | 2541 | 846 | 0 | 884 | | |
| | | | amplifying primer, SEQ ID 7. | | | | | | | | |
| 669, 670 | AEX25100 | 1.00E−145 | Enterobacter cloacae protein amino acid | AEH55030 | 0.12 | 2307 | 768 | 0 | 765 | 68 | |
| | | | sequence - SEQ ID 5666. | | | | | | | | |
| 671, 672 | AEG60856 | 1.00E−147 | Enterobacter cloacae protein amino acid | AEH55475 | 8.00E−05 | 1572 | 523 | 0 | 523 | | |
| | | | sequence - SEQ ID 5666. | | | | | | | | |
| 673, 674 | AAW53957 | 0 | Streptomyces lividans alpha-L- | AEH35455 | 3.00E−10 | 1506 | 501 | 0 | 502 | 62 | |
| | | | arabinofuranosidase, abfA reporter gene. | 0 | 489 | | | | | | |
| 675, 676 | ADS28234 | 1.00E−138 | Bacterial polypeptide #23667. | ADT43018 | 3.00E−10 | 1482 | 493 | | | | |
| 677, 678 | ADS27294 | 1.00E−171 | Microbulbifer degradans cellulase system | AEH81970 | 2.00E−08 | 1566 | 521 | 0 | 521 | 57 | |
| | | | protein - SEQ ID 8. | | | | | | | | |
| 679, 680 | ADJ34876 | 1.00E−59 | S roseosporus daptomycin biosynthesis gene | ADJ72366 | 0.19 | 1020 | 339 | 0 | 336 | 36 | |
| | | | cluster protein #20. | | | | | | | | |
| 681, 682 | AGI25538 | 1.00E−108 | Plant full length insert polynucleotide seqid | ADX50876 | 7.00E−06 | 2094 | 697 | 0 | 709 | | |
| | | | 4980. | | | | | | | | |
| 683, 684 | AAB10913 | 1.00E−110 | Streptomyces sp. arabinofuranosidase DNA | AAA71999 | 4.00E−04 | 1983 | 660 | 0 | 660 | 60 | |
| | | | SEQ ID NO: 2. | | | | | | | | |
| 685, 686 | ADS28234 | 1.00E−172 | Xylanase from an environmental sample seq id | ADJ34919 | 2.00E−16 | 2637 | 878 | 0 | 521 | 33 | |
| | | | 14. | | | | | | | | |
| 687, 688 | ADJ34868 | 4.00E−58 | Chlorella sorokiniana EST SEQ ID NO 9395. | AJP88135 | 2.5 | 843 | 280 | 0 | 286 | 58 | |
| 689, 690 | AAY01076 | 1.00E−102 | Human OPG (osteoprotegerin) K108N protein | ABS54850 | 1.00E−113 | 1350 | 449 | 0 | 394 | 65 | |
| | | | mutant. | | | | | | | | |
| 691, 692 | ADN25476 | 0 | Bacterial polypeptide #23667. | ADS56142 | 0 | 2643 | 880 | 0 | 880 | 100 | |
| 693, 694 | ADR51303 | 1.00E−141 | Streptococcus sp. H021 Orf2, oxidoreductase. | AAD47222 | 1.1 | 1404 | 467 | 0 | 471 | 56 | |
| 695, 696 | AEH81913 | 1.00E−125 | Microbulbifer degradans cellulase system | AEH81914 | 8.00E−04 | 975 | 324 | 0 | 324 | | |
| | | | protein - SEQ ID 8. | | | | | | | | |
| 697, 698 | AEH81913 | 1.00E−127 | LRTM4 protein #SEQ ID 2. | ACC83217 | 0.18 | 972 | 323 | 0 | 324 | | |
| 699, 700 | ADR51269 | 0 | Anti-biofilm polypeptide #100. | ADR51268 | 0 | 2163 | 720 | 0 | 705 | 58 | |
| 718, 719 | ADJ34800 | 0 | Xylanase from an environmental sample seq id | ADJ34799 | 0 | 1110 | 369 | 1110 | 369 | | |
| | | | 14. | | | | | | | | |
| 720, 721 | ADS27945 | 0.39 | Human immune system associated gene SEQ | ABL32292 | 0.25 | 1299 | 432 | 0 | 444 | | |
| | | | ID. NO: 59. | | | | | | | | |

The initial source of selected exemplary polypeptides and nucleic acids of this invention are:

| SEQ ID NO: | Source |
|---|---|
| 473 | Glycine max glycinin GY1 signal sequence |
| 474 | ER retention sequence |
| 475 | sporamin vacuolar targeting sequence |
| 476 | transit peptide from ferredoxin-NADP+ reductase (FNR) |
| | of Cyanophora paradoxa |
| 477 | protein storage vacuole (PSV) sequence from |
| | b-conglycinin |
| 478 | gamma zein 27 kD signal sequence |
| 479 | vacuole sequence domain (VSD) from barley polyamine |
| | oxidase |
| 480 | dicot optimized SEQ ID NO: 359 |
| 481 | dicot optimized SEQ ID NO: 357 |
| 482 | dicot optimized SEQ ID NO: 167 |
| 483 | monocot optimized SEQ ID NO: 359 |
| 484 | monocot optimized SEQ ID NO: 357 |
| 485 | monocot optimized SEQ ID NO: 167 |
| 486 | monocot optimized SEQ ID NO: 33 |
| 487 | dicot optimized SEQ ID NO: 33 |
| 488 | Cestrum yellow leaf curl virus promoter plus leader |
| 701 | from SEQ ID NO: 360 (D2150-3WO) |
| 702 | from SEQ ID NO: 371 (D2150-3WO) |
| 703 | from SEQ ID NO: 606 (D2150-3WO) |
| 704 | Thermobifida fusca GH6 (Genbank YP_289135) |
| 705 | Saccharophagus degradans (Genbank YP_527744) |
| 706 | Xylella fastidiosa (Genbank NP_780034.1) |
| 1, 2 | Unknown |
| 101, 102 | Unknown |
| 103, 104 | Unknown |
| 105, 106 | Unknown |
| 107, 108 | Unknown |
| 109, 110 | Unknown |
| 11, 12 | Teredinibacter |
| 111, 112 | Unknown |
| 113, 114 | Unknown |
| 115, 116 | Unknown |
| 117, 118 | Unknown |
| 119, 120 | Unknown |
| 121, 122 | Unknown |
| 123, 124 | Unknown |
| 125, 126 | Unknown |
| 127, 128 | Unknown |
| 129, 130 | Unknown |
| 13, 14 | Unknown |
| 131, 132 | Unknown |
| 133, 134 | Unknown |
| 135, 136 | Unknown |
| 137, 138 | Unknown |
| 139, 140 | Unknown |
| 141, 142 | Unknown |
| 143, 144 | Unknown |
| 145, 146 | Unknown |
| 147, 148 | Unknown |
| 149, 150 | Unknown |
| 15, 16 | Bacteria |
| 151, 152 | Unknown |
| 153, 154 | Unknown |
| 155, 156 | Unknown |
| 157, 158 | Unknown |
| 159, 160 | Unknown |
| 161, 162 | Unknown |
| 163, 164 | Unknown |
| 165, 166 | Unknown |
| 167, 168 | Unknown |
| 169, 170 | Unknown |
| 17, 18 | Bacteria |
| 171, 172 | Unknown |
| 173, 174 | Unknown |
| 175, 176 | Unknown |
| 177, 178 | Unknown |
| 179, 180 | Unknown |
| 181, 182 | Unknown |
| 183, 184 | Unknown |
| 185, 186 | Unknown |
| 187, 188 | Unknown |
| 189, 190 | Unknown |
| 19, 20 | Unknown |
| 191, 192 | Unknown |
| 193, 194 | Unknown |
| 195, 196 | Unknown |
| 197, 198 | Unknown |
| 199, 200 | Unknown |
| 201, 202 | Unknown |
| 203, 204 | Unknown |
| 205, 206 | Unknown |
| 207, 208 | Unknown |
| 209, 210 | Unknown |
| 21, 22 | Unknown |
| 211, 212 | Unknown |
| 213, 214 | Unknown |
| 215, 216 | Unknown |
| 217, 218 | Unknown |
| 219, 220 | Unknown |
| 221, 222 | Unknown |
| 223, 224 | Unknown |
| 225, 226 | Unknown |
| 227, 228 | Unknown |
| 229, 230 | Unknown |
| 23, 24 | Unknown |
| 231, 232 | Unknown |
| 233, 234 | Unknown |
| 235, 236 | Unknown |
| 237, 238 | Unknown |
| 239, 240 | Unknown |
| 241, 242 | Unknown |
| 243, 244 | Unknown |
| 245, 246 | Unknown |
| 247, 248 | Unknown |
| 249, 250 | Unknown |
| 25, 26 | Unknown |
| 251, 252 | Unknown |
| 253, 254 | Unknown |
| 255, 256 | Unknown |
| 257, 258 | Unknown |
| 259, 260 | Unknown |
| 261, 262 | Unknown |
| 263, 264 | Unknown |
| 265, 266 | Unknown |
| 267, 268 | Fungus |
| 269, 270 | Unknown |
| 27, 28 | Agaricus bisporus ATCC 62489 |
| 271, 272 | Unknown |
| 273, 274 | Unknown |
| 275, 276 | Unknown |
| 277, 278 | Unknown |
| 279, 280 | Unknown |
| 281, 282 | Fungus |
| 283, 284 | Unknown |
| 285, 286 | Unknown |
| 287, 288 | Unknown |
| 289, 290 | Unknown |
| 29, 30 | Agaricus bisporus ATCC 62489 |
| 291, 292 | Unknown |
| 293, 294 | Unknown |
| 295, 296 | Unknown |
| 297, 298 | Unknown |
| 299, 300 | Unknown |
| 3, 4 | Unknown |
| 301, 302 | Unknown |
| 303, 304 | Unknown |
| 305, 306 | Unknown |
| 307, 308 | Unknown |
| 309, 310 | Unknown |
| 31, 32 | Unknown |
| 311, 312 | Unknown |
| 313, 314 | Unknown |
| 315, 316 | Unknown |
| 317, 318 | Unknown |
| 319, 320 | Unknown |
| 321, 322 | Unknown |
| 323, 324 | Unknown |
| 325, 326 | Unknown |
| 327, 328 | Unknown |
| 329, 330 | Unknown |
| 33, 34 | Fungus |
| 331, 332 | Unknown |
| 333, 334 | Unknown |
| 335, 336 | Unknown |
| 337, 338 | Unknown |
| 339, 340 | Unknown |
| 341, 342 | Unknown |
| 343, 344 | Unknown |
| 345, 346 | Unknown |
| 347, 348 | Unknown |
| 349, 350 | Unknown |
| 35, 36 | Cochliobolus heterostrophus ATCC 48331 |
| 351, 352 | Unknown |
| 353, 354 | Unknown |
| 355, 356 | Unknown |
| 357, 358 | Fungus |
| 359, 360 | Fungus |
| 361, 362 | Clostridium thermocellum ATCC 27405 |
| 363, 364 | Clostridium thermocellum ATCC 27405 |
| 365, 366 | Clostridium thermocellum ATCC 27405 |
| 367, 368 | Unknown |
| 369-371 | Fungus |
| 37, 38 | Clostridium thermocellum ATCC 27405 |
| 372-374 | Botrytis cinerea ATCC 204446 |
| 375-377 | Fusarium verticillioides GZ3639 |
| 378-380 | Fungus |
| 381-383 | Fungus |
| 384-386 | Fungus |
| 387-389 | Fungus |
| 39, 40 | Unknown |
| 390-392 | Fungus |
| 393-395 | Fungus |
| 396-398 | Fungus |
| 399-401 | Fungus |
| 402-404 | Fungus |
| 405-407 | Fungus |
| 408-410 | Fungus |
| 41, 42 | Unknown |
| 411-413 | Fungus |
| 414-416 | Fungus |
| 417-419 | Fungus |
| 420-422 | Agaricus bisporus ATCC 62489 |
| 423, 424 | Unknown |
| 425, 426 | Unknown |
| 427, 428 | Unknown |
| 429, 430 | Unknown |
| 43, 44 | Unknown |
| 431, 432 | Unknown |
| 433, 434 | Unknown |
| 435, 436 | Unknown |
| 437, 438 | Unknown |
| 439, 440 | Unknown |
| 441, 442 | Unknown |
| 443, 444 | Bacteria |
| 445, 446 | Unknown |
| 447, 448 | Unknown |
| 449, 450 | Unknown |
| 45, 46 | Unknown |
| 451, 452 | Unknown |
| 453, 454 | Unknown |
| 455, 456 | Thermobifida fusca |
| 457, 458 | Thermobifida fusca |
| 459, 460 | Bacteria |
| 461, 462 | Bacteria |
| 463, 464 | Unknown |
| 465, 466 | Unknown |
| 467, 468 | Unknown |
| 469, 470 | Unknown |
| 47, 48 | Unknown |
| 471, 472 | Streptomyces coelicolor |
| 489, 490 | Fungus |
| 49, 50 | Unknown |
| 491, 492 | Fungus |
| 493, 494, 707 | Fungus |
| 495, 496, 710 | Fungus |
| 497, 498, 711 | Fungus |
| 499, 500, 712 | Fungus |
| 5, 6 | Unknown |
| 501, 502, 713 | Fungus |
| 503, 504, 714 | Fungus |
| 505, 506, 715 | Fungus |
| 507, 508, 716 | Fungus |
| 509, 510, 717 | Fungus |
| 51, 52 | Unknown |
| 511, 512, 708 | Fungus |
| 513, 514, 709 | Fungus |
| 515, 516 | Clostridium thermocellum |
| 517, 518 | Fungus |
| 519, 520 | Fungus |
| 521, 522 | Fungus |
| 523, 524 | Fungus |
| 525, 526 | Unknown |
| 527, 528 | Unknown |
| 529, 530 | Clostridium thermocellum |
| 53, 54 | Unknown |
| 531, 532 | Unknown |
| 533, 534 | Unknown |
| 535, 536 | Thermococcus alcaliphilus |
| 537, 538 | Thermotoga maritima MSB8 |
| 539, 540 | Unknown |
| 541, 542 | Unknown |
| 543, 544 | Unknown |
| 545, 546 | Unknown |
| 547, 548 | Unknown |
| 549, 550 | Unknown |
| 55, 56 | Unknown |
| 551, 552 | Unknown |
| 553, 554 | Unknown |
| 555, 556 | Pyrococcus furiosus VC1 |
| 557, 558 | Cochliobolus heterostrophus ATCC 48331 |
| 559, 560 | Unknown |
| 561, 562 | Unknown |
| 563, 564 | Unknown |
| 565, 566 | Unknown |
| 567, 568 | Unknown |
| 569, 570 | |
| 57, 58 | Unknown |
| 571, 572 | Unknown |
| 573, 574 | Unknown |
| 575, 576 | Bacteria |
| 577, 578 | Unknown |
| 579, 580 | |
| 581, 582 | Unknown |
| 583, 584 | Unknown |
| 585, 586 | Unknown |
| 587, 588 | Unknown |
| 589, 590 | Unknown |
| 59, 60 | Unknown |
| 591, 592 | Unknown |
| 593, 594 | Unknown |
| 595, 596 | Unknown |
| 597, 598 | Trichoderma reesei ATCC 13631 |
| 599, 600 | Trichoderma reesei ATCC 13631 |
| 601, 602 | Fungus |
| 603, 604 | Fungus |
| 605, 606 | Fungus |
| 607, 608 | Fungus |
| 609, 610 | Fungus |
| 61, 62 | Unknown |
| 611, 612 | Unknown |
| 613, 614 | Unknown |
| 615, 616 | Fungus |
| 617, 618 | Fungus |
| 619, 620 | Fungus |
| 621, 622 | Unknown |
| 623, 624 | Unknown |
| 625, 626 | Unknown |
| 627, 628 | Cochliobolus heterostrophus ATCC 48331 |
| 629, 630 | Cochliobolus heterostrophus ATCC 48331 |
| 63, 64 | Unknown |
| 631, 632 | Cochliobolus heterostrophus ATCC 48331 |
| 633, 634 | Cochliobolus heterostrophus ATCC 48331 |
| 635, 636 | Cochliobolus heterostrophus ATCC 48331 |
| 637, 638 | Cochliobolus heterostrophus ATCC 48331 |
| 639, 640 | Unknown |
| 641, 642 | Unknown |
| 643, 644 | Unknown |
| 645, 646 | Unknown |
| 647, 648 | Unknown |
| 649, 650 | Cochliobolus heterostrophus ATCC 48331 |
| 65, 66 | Unknown |
| 651, 652 | Cochliobolus heterostrophus ATCC 48331 |
| 653, 654 | Unknown |
| 655, 656 | Unknown |
| 657, 658 | Unknown |
| 659, 660 | Cochliobolus heterostrophus ATCC 48331 |
| 661, 662 | Cochliobolus heterostrophus ATCC 48331 |
| 663, 664 | Cochliobolus heterostrophus ATCC 48331 |
| 665, 666 | Cochliobolus heterostrophus ATCC 48331 |
| 667, 668 | Cochliobolus heterostrophus ATCC 48331 |
| 669, 670 | Unknown |
| 67, 68 | Unknown |
| 671, 672 | Unknown |
| 673, 674 | Unknown |
| 675, 676 | Unknown |
| 677, 678 | Unknown |
| 679, 680 | Unknown |
| 681, 682 | Unknown |
| 683, 684 | Unknown |
| 685, 686 | Unknown |
| 687, 688 | Cochliobolus heterostrophus ATCC 48331 |
| 689, 690 | Cochliobolus heterostrophus ATCC 48331 |
| 69, 70 | Unknown |
| 691, 692 | Thermobifida fusca YX BAA-629 |
| 693, 694 | Unknown |
| 695, 696 | Unknown |
| 697, 698 | Unknown |
| 699, 700 | Unknown |
| 7, 8 | Unknown |
| 71, 72 | Unknown |
| 718, 719 | Unknown |
| 720, 721 | Cochliobolus heterostrophus ATCC 48331 |
| 73, 74 | Unknown |
| 75, 76 | Unknown |
| 77, 78 | Unknown |
| 79, 80 | Unknown |
| 81, 82 | Unknown |
| 83, 84 | Unknown |
| 85, 86 | Unknown |
| 87, 88 | Unknown |
| 89, 90 | Clostridium thermocellum ATCC 27405 |
| 9, 10 | Unknown |
| 91, 92 | Unknown |
| 93, 94 | Unknown |
| 95, 96 | Unknown |
| 97, 98 | Unknown |
| 99, 100 | Unknown |

The invention also includes methods for discovering, identifying or isolated new lignocellulosic enzymes, including cellulase, endoglucanase, cellobiohydrolase, beta-glucosidase, xylanase, mannanse, β-xylosidase and/or arabinofuranosidase polypeptide sequences using the nucleic acids of the invention. The invention also includes methods for inhibiting the expression of the lignocellulosic enzyme encoding genes and transcripts using the nucleic acids of the invention.

Also provided are methods for modifying the nucleic acids of the invention, including making variants of nucleic acids of the invention, by, e.g., synthetic ligation reassembly, optimized directed evolution system and/or saturation mutagenesis such as GENE SITE SATURATION MUTAGENESIS (or GSSM). The term “saturation mutagenesis”, GENE SITE SATURATION MUTAGENESIS or GSSM includes a method that uses degenerate oligonucleotide primers to introduce point mutations into a polynucleotide, as described in detail, below. The term “optimized directed evolution system” or “optimized directed evolution” includes a method for reassembling fragments of related nucleic acid sequences, e.g., related genes, and explained in detail, below. The term “synthetic ligation reassembly” or “SLR” includes a method of ligating oligonucleotide fragments in a non-stochastic fashion, and explained in detail, below. The term “variant” refers to polynucleotides or polypeptides of the invention modified at one or more base pairs, codons, introns, exons, or amino acid residues (respectively) yet still retain the biological activity of a lignocellulosic enzyme, e.g., a glycosyl hydrolase, cellulase, endoglucanase, cellobiohydrolase, beta-glucosidase, xylanase, mannanse, β-xylosidase and/or arabinofuranosidase of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM and any combination thereof.

The nucleic acids of the invention can be made, isolated and/or manipulated by, e.g., cloning and expression of cDNA libraries, amplification of message or genomic DNA by PCR, and the like. For example, exemplary sequences of the invention were initially derived from environmental sources. Thus, in one aspect, the invention provides the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme-encoding nucleic acids, and the polypeptides encoded by them, having a common novelty in that they are derived from a common source, e.g., an environmental, mixed culture, or a bacterial source.

In practicing the methods of the invention, homologous genes can be modified by manipulating a template nucleic acid, as described herein. The invention can be practiced in conjunction with any method or protocol or device known in the art, which are well described in the scientific and patent literature. A “coding sequence of” or a “nucleotide sequence encoding” a particular polypeptide or protein, is a nucleic acid sequence which is transcribed and translated into a polypeptide or protein when placed under the control of appropriate regulatory sequences. The term “gene” means the segment of DNA involved in producing a polypeptide chain; it includes regions preceding and following the coding region (leader and trailer) as well as, where applicable, intervening sequences (introns) between individual coding segments (exons). A promoter sequence is “operably linked to” a coding sequence when RNA polymerase which initiates transcription at the promoter will transcribe the coding sequence into mRNA. “Operably linked” as used herein refers to a functional relationship between two or more nucleic acid (e.g., DNA) segments. It can refer to the functional relationship of transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. In one aspect, promoter transcriptional regulatory sequences are operably linked to a transcribed sequence (e.g., a sequence of the invention) and are physically contiguous to the transcribed sequence, i.e., they are cis-acting. However, some transcriptional regulatory sequences, such as enhancers, need not be physically contiguous or located in close proximity to the coding sequences whose transcription they enhance. Promoters used to “drive” transcription of nucleic acids of the invention include, e.g., a viral, bacterial, mammalian or plant promoter; or, a plant promoter; or, a potato, rice, corn, wheat, tobacco or barley promoter; or, a constitutive promoter or a CaMV35S promoter; or, an inducible promoter; or, a tissue-specific promoter or an environmentally regulated or a developmentally regulated promoter; or, a seed-specific, a leaf-specific, a root-specific, a stem-specific or an abscission-induced promoter; or, a seed preferred promoter, a maize gamma zein promoter or a maize ADP-gpp promoter.

One aspect of the invention is an isolated, synthetic or recombinant nucleic acid comprising one of the sequences of the invention, or a fragment comprising at least 10, 15, 20, 25, 30, 35, 40, 50, 75, 100, 150, 200, 300, 400, or 500 or more consecutive bases of a nucleic acid of the invention. The isolated, synthetic or recombinant nucleic acids may comprise DNA, including cDNA, genomic DNA and synthetic DNA. The DNA may be double-stranded or single-stranded and if single stranded may be the coding strand or non-coding (anti-sense) strand. Alternatively, the isolated, synthetic or recombinant nucleic acids comprise RNA.

The isolated, synthetic or recombinant nucleic acids of the invention may be used to prepare one of the polypeptides of the invention, or fragments comprising at least 5, 10, 15, 20, 25, 30, 35, 40, 50, 75, 100, or 150 or more consecutive amino acids of one of the polypeptides of the invention. Accordingly, another aspect of the invention is an isolated, synthetic or recombinant nucleic acid which encodes one of the polypeptides of the invention, or fragments comprising at least 5, 10, 15, 20, 25, 30, 35, 40, 50, 75, 100, or 150 or more consecutive amino acids of one of the polypeptides of the invention. The to coding sequences of these nucleic acids may be identical to one of the coding sequences of one of the nucleic acids of the invention or may be different coding sequences which encode one of the of the invention having at least 5, 10, 15, 20, 25, 30, 35, 40, 50, 75, 100, or 150 or more consecutive amino acids of one of the polypeptides of the invention, as a result of the redundancy or degeneracy of the genetic code. The genetic code is well known to those of skill in the art and can be obtained, e.g., on page 214 of B. Lewin, Genes VI, Oxford University Press, 1997.

The nucleic acids encoding polypeptides of the invention include but are not limited to: the coding sequence of a nucleic acid of the invention and additional coding sequences, such as leader sequences or proprotein sequences and non-coding sequences, such as introns or non-coding sequences 5′ and/or 3′ of the coding sequence. Thus, as used herein, the term “polynucleotide encoding a polypeptide” encompasses a polynucleotide which includes the coding sequence for the polypeptide as well as a polynucleotide which includes additional coding and/or non-coding sequence.

In one aspect, the nucleic acid sequences of the invention are mutagenized using conventional techniques, such as site directed mutagenesis, or other techniques familiar to those skilled in the art, to introduce silent changes into the polynucleotides o of the invention. As used herein, “silent changes” include, for example, changes which do not alter the amino acid sequence encoded by the polynucleotide. Such changes may be desirable in order to increase the level of the polypeptide produced by host cells containing a vector encoding the polypeptide by introducing codons or codon pairs which occur frequently in the host organism.

The invention also relates to polynucleotides which have nucleotide changes which result in amino acid substitutions, additions, deletions, fusions and truncations in the polypeptides of the invention. Such nucleotide changes may be introduced using techniques such as site directed mutagenesis, random chemical mutagenesis, exonuclease III deletion and other recombinant DNA techniques. Alternatively, such nucleotide changes may be naturally occurring allelic variants which are isolated by identifying nucleic acids which specifically hybridize to probes comprising at least 10, 15, 20, 25, 30, 35, 40, 50, 75, 100, 150, 200, 300, 400, or 500 consecutive bases of one of the sequences of the invention (or the sequences complementary thereto) under conditions of high, moderate, or low stringency as provided herein.

General Techniques

The nucleic acids used to practice this invention, whether RNA, siRNA, miRNA, antisense nucleic acid, cDNA, genomic DNA, vectors, viruses or hybrids thereof, may be isolated from a variety of sources, genetically engineered, amplified, and/or expressed/ generated recombinantly. Recombinant polypeptides (e.g., the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes) generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

This invention encompasses “nucleic acid” or “nucleic acid sequence” as oligonucleotides, nucleotides, polynucleotides, fragments of any of these, to DNA, cDNA, gDNA, RNA (message), RNAi, etc. of genomic or synthetic origin or derivation, any of which may be single-stranded or double-stranded and may represent a sense or antisense (complementary) strand, to peptide nucleic acid (PNA), or to any DNA-like or RNA-like material, natural or synthetic in origin. This invention encompasses “nucleic acids” or “nucleic acid sequences” including any sense or antisense sequences, peptide nucleic acids (PNA), any DNA-like or RNA-like material, natural or synthetic in origin, including, e.g., iRNA, ribonucleoproteins (e.g., e.g., double stranded iRNAs, e.g., iRNPs). This invention encompasses nucleic acids, i.e., oligonucleotides, containing known analogues of natural nucleotides. This invention encompasses nucleic-acid-like structures with synthetic backbones, which is one possible embodiment of the synthetic nucleic acids of the invention; see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. “Oligonucleotide” includes either a single stranded polydeoxynucleotide or two complementary polydeoxynucleotide strands which may be chemically synthesized. This invention encompasses synthetic nucleic acids and/or oligonucleotides that have no 5′ phosphate; thus will not ligate to another oligonucleotide without adding a phosphate with an ATP in the presence of a kinase; a synthetic oligonucleotide can ligate to a fragment that has not been dephosphorylated.

Alternative structures of synthetic nucleic acids and/or oligonucleotides, and methods for making them, are well known in the art and all are incorporated for making and using this invention.

The invention provides “recombinant” polynucleotides (and proteins), and in one is aspect the recombinant nucleic acids are adjacent to a “backbone” nucleic acid, which it is not adjacent in its natural environment. In one aspect, to be “enriched” the nucleic acids will represent about 1%, 5%, 10%, 15%, 20%, 25% or more of the number of nucleic acid inserts in a population of nucleic acid backbone molecules. In one aspect, backbone molecules comprise nucleic acids such as expression vectors, self-replicating nucleic acids, viruses, integrating nucleic acids and other vectors or nucleic acids used to maintain or manipulate a nucleic acid insert of interest. In one aspect, the enriched nucleic acids represent about 1%, 5%, 10%, 15%, 20%, 25% or more of the number of nucleic acid inserts in the population of recombinant backbone molecules. In one aspect, the enriched nucleic acids represent about 50%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% or more of the number of nucleic acid inserts in the population of recombinant backbone molecules. In a one aspect, the enriched nucleic acids represent about 90%, 91%, 92%, 93%, 94%, 95%, 96%, 97%, 98%, 99% or more of the number of nucleic acid inserts in the population of recombinant backbone molecules.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lad, lacZ, T3, T7, gpt, lambda PR, PL and trp. Exemplary is eukaryotic promoters include CMV immediate early, HSV thymidine kinase, early and late SV40, LTRs from retrovirus, and mouse metallothionein I.

As used herein, the term “promoter” includes all sequences capable of driving transcription of a coding sequence in a cell, e.g., a plant or animal cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Tissue-specific” promoters are transcriptional control elements that are only active in particular cells or tissues or organs, e.g., in plants or animals. Tissue-specific regulation may be achieved by certain intrinsic factors which ensure that genes encoding proteins specific to a given tissue are expressed. Such factors are known to exist in mammals and plants so as to allow for specific tissues to develop.

Promoters suitable for expressing a polypeptide in bacteria include the E. coli lac or trp promoters, the lad promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used. Promoters suitable for expressing the polypeptide or fragment thereof in bacteria include the E. coli lac or trp promoters, the lacI promoter, the lacZ promoter, the T3 promoter, the T7 promoter, the gpt promoter, the lambda P_{R} promoter, the lambda P_{L} promoter, promoters from operons encoding glycolytic enzymes such as 3-phosphoglycerate kinase (PGK) and the acid phosphatase promoter. Fungal promoters include the α-factor promoter. Eukaryotic promoters include the CMV immediate early promoter, the HSV thymidine kinase promoter, heat shock promoters, the early and late SV40 promoter, LTRs from retroviruses and the mouse metallothionein-I promoter. Other promoters known to control expression of genes in prokaryotic or eukaryotic cells or their viruses may also be used.

Tissue-Specific Plant Promoters

The invention provides expression cassettes that can be expressed in a plant part (e.g., seed, leaf, root or seed) or tissue-specific manner, e.g., that can express a lignocellulosic enzyme of the invention, e.g., a glycosyl hydrolase, cellulase, endoglucanase, cellobiohydrolase, beta-glucosidase, xylanase, mannanse, β-xylosidase and/or arabinofuranosidase enzyme of the invention in a part-specific or tissue-specific manner. The invention also provides plants or seeds that express a lignocellulosic enzyme of the invention in a stage-specific and/or tissue-specific manner. The tissue-specificity can be seed specific, stem specific, leaf specific, root specific, fruit specific and the like. The nucleic acids of the invention can be operably linked to any promoter, e.g., as in an expression cassette (such as a vector, plasmid, and the like) that provides very high expression in a plant, plant part (e.g., a root, stem, seed or fruit) or plant seed, including promoters that are active in any part of the plant (but also expressing at a high level in at least one part, if not all, part of the plant), or alternatively, the promoter can express a nucleic acid of the invention at a high level in less than all of the plant, e.g., in a tissue-specific manner. In one aspect, the promoter is constitutive and results in a constitutive high level of expression; alternatively, the promoter can be inducible, i.e., it can be induced to produce a high level of expression of a nucleic acid of the invention, e.g., by application of a chemical, infection of an agent that makes an inducing chemical or protein, by a normal or induced maturation or growth process where the plant endogenously turns certain genes and promoters on and off.

In one aspect, a constitutive promoter such as the CaMV 35S promoter can be used for expression in specific parts of the plant or seed or throughout the plant. For example, for overexpression, a plant promoter fragment can be employed which will direct expression of a nucleic acid in some or all tissues of a plant, e.g., a regenerated plant. Such promoters are referred to herein as “constitutive” promoters and are active under most environmental conditions and states of development or cell differentiation.

Examples of constitutive promoters include the cauliflower mosaic virus (CaMV) 35S transcription initiation region (the Cauliflower Mosaic Virus promoter; see, e.g., U.S. Pat. No. 5,110,732); the 1′- or 2′-promoter derived from T-DNA of Agrobacterium tumefaciens; and other transcription initiation regions from various plant genes known to those of skill.

Promoters, enhancers and/or other transcriptional or translations regulatory motifs that can be used to practice this invention include those from any plant, animal or microorganism gene known in the art, e.g., including ACT11 from Arabidopsis (Huang (1996) Plant Mol. Biol. 33:125-139); Cat3 from Arabidopsis (GenBank No. U43147, Zhong (1996) Mol. Gen. Genet. 251:196-203); the gene encoding stearoyl-acyl carrier protein desaturase from Brassica napus (Genbank No. X74782, Solocombe (1994) Plant Physiol. 104:1167-1176); GPc1 from maize (GenBank No. X15596; Martinez (1989) J. Mol. Biol 209:551-565); the Gpc2 from maize (GenBank No. U45855, Manjunath (1997) Plant Mol. Biol. 33:97-112); plant promoters described in U.S. Pat. Nos. 4,962,028; 5,633,440.

The invention uses tissue-specific, inducible or constitutive promoters and/or enhancers derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139). In one aspect, the invention uses the cestrum yellow leaf curling virus promoter as described, e.g., in U.S. Pat. No. 7,166,770; 10^{th} IAPTC&B Congress “Plant Biotechnology 2002 and beyond.” Kononova, et al., p. 237-238. Jun. 24, 2002. In one aspect, the invention uses the corn (maize) endosperm specific promoter as described, e.g., in U.S. Pat. No. 7,157,623. In one aspect, the invention uses promoters that regulate the expression of zinc finger proteins, as described, e.g., in U.S. Pat. No. 7,151,201. In one aspect, to the invention uses the corn (maize) promoters as described, e.g., in U.S. Pat. No. 7,138,278. In one aspect, the invention uses “arcelin” promoters (including, e.g., the Arcelin-3, Arcelin-4 and Arcelin-5 promoters) capable of transcribing a heterologous nucleic acid sequence at high levels in plants, as described, e.g., in U.S. Pat. No. 6,927,321. In one aspect, the invention uses plant embryo-specific promoters, as described, e.g., in U.S. Pat. Nos. 6,781,035; 6,235,975. In one aspect, the invention uses promoters for potato tuber specific expression, as described, e.g., in U.S. Pat. No. 5,436,393. In one aspect, the invention uses promoters for leaf-specific expression, as described, e.g., in U.S. Pat. No. 6,229,067. In one aspect, the invention uses promoters for mesophyll-specific expression, as described, e.g., in U.S. Pat. No. 6,610,840.

Seed-preferred regulatory sequences (e.g., seed-specific promoters) are described e.g., in U.S. Pat. Nos. 7,081,566; 7,081,565; 7,078,588; 6,566,585; 6,642,437; 6,410,828; 6,066,781; 5,889,189; 5,850,016.

In one aspect, the plant promoter directs expression of the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme-expressing nucleic acid in a specific tissue, organ or cell type (i.e. tissue-specific promoters) or may be otherwise under more precise environmental or developmental control or under the control of an inducible promoter. Examples of environmental conditions that may affect transcription include anaerobic conditions, elevated temperature, the presence of light, or sprayed with chemicals/hormones. For example, the invention incorporates the drought-inducible promoter of maize (Busk (1997) supra); the cold, drought, and high salt inducible promoter from potato (Kirch (1997) Plant Mol. Biol. 33:897 909).

Any tissue-specific regulated coding sequence, genes and/or transcriptional regulatory sequence (including promoters and enhancers) from any plant can be used to practice this invention; including, e.g., tissue-specific promoters and enhancers and coding sequence; or, promoters and enhancers or genes, including the coding sequences or genes encoding the seed storage proteins, such as napin, cruciferin, beta-conglycinin, and phaseolin, zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development (such as Bce4, see, for example, EP 255378 and Kridl (1991) Seed Science Research 1:209), and promoters and enhancers associated with these genes and protein coding sequences.

Exemplary tissue-specific promoters and enhancers which can be used to practice this invention include tissue-specific promoters and enhancers from the following plant genes: lectin (see, e.g., Vodkin (1983) Prog. Clin. Biol. Res. 138:87; Lindstrom (1990) Der. Genet. 11:160), corn alcohol dehydrogenase 1 (see, e.g., Kyozuka (1994) Plant Cell 6(6):799-810; Dennis (1985) Nucleic Acids Res. 13(22):7945-57); corn light harvesting complex (see, e.g., Simpson, (1986) Science, 233:34; Bansal (1992) Proc. Natl. Acad. Sci. USA 89:3654), corn heat shock protein (see, e.g., Odell et al., (1985) Nature, 313:810; pea small subunit RuBP carboxylase (see, e.g., Poulsen et al., (1986) Mol. Gen. Genet., 205:193-200; Cashmore et al., (1983) Gen. Eng. of Plants, Plenum Press, New York, 29-38), Ti plasmid mannopine synthase (see, e.g., Langridge et al., (1989) Proc. Natl. Acad. Sci. USA, 86:3219-3223), Ti plasmid nopaline synthase (Langridge et al., (1989) Proc. Natl. Acad. Sci. USA, 86:3219-3223), petunia chalcone isomerase (see, e.g., vanTunen (1988) EMBO J. 7:1257), bean glycine rich protein 1 (see, e.g., Keller (1989) Genes Dev. 3:1639), truncated CaMV 35s (see, e.g., Odell (1985) Nature 313:810), potato patatin (see, e.g., Wenzler (1989) Plant Mol. Biol. 13:347; root cell (see, e.g., Yamamoto (1990) Nucleic Acids Res. 18:7449), maize zein (see, e.g., Reina (1990) Nucleic Acids Res. 18:6425; Kriz (1987) Mol. Gen. Genet. 207:90; Wandelt (1989) Nucleic Acids Res., 17:2354; Langridge (1983) Cell, 34:1015; Reina (1990) Nucleic Acids Res., 18:7449), ADP-gpp promoter (see, e.g., U.S. Pat. No. 7,102,057); globulin-1 (see, e.g., Belanger (1991) Genetics 129:863), α-tubulin, cab (see, e.g., Sullivan (1989) Mol. Gen. Genet., 215:431), PEPCase (see e.g., Hudspeth & Grula, (1989) Plant Molec. Biol., 12:579-589); R gene complex-associated promoters (see, e.g., Chandler (1989) Plant Cell 1:1175); chalcone synthase promoters (see, e.g., Franken (1991) EMBO J., 10:2605); and/or the soybean heat-shock gene promoter, see, e.g., Lyznik (1995) Plant J. 8(2):177-86.

In one aspect the invention uses seed-specific transcriptional regulatory elements for seed-specific expression, e.g., including use of the pea vicilin promoter (see, e.g., Czako (1992) Mol. Gen. Genet., 235:33; see also U.S. Pat. No. 5,625,136. Other useful promoters for expression in mature leaves are those that are switched on at the onset of senescence, such as the SAG promoter from Arabidopsis (see, e.g., Gan (1995) Science 270:1986.

In one aspect the invention uses fruit-specific promoters expressed at or during anthesis through fruit development, at least until the beginning of ripening, as described, e.g., in U.S. Pat. No. 4,943,674. In one aspect the invention uses cDNA clones that are preferentially expressed in cotton fiber, as described, e.g., in John (1992) Proc. Natl. Acad. Sci. USA 89:5769. In one aspect the invention uses cDNA clones from tomato displaying differential expression during fruit development, as described, e.g., in Mansson et al., Gen. Genet., 200:356 (1985), Slater et al., Plant Mol. Biol., 5:137 (1985)). In one aspect the invention uses the promoter for polygalacturonase gene, which is active in fruit ripening; the polygalacturonase gene is described, e.g., in U.S. Pat. Nos. 4,535,060; 4,769,061; 4,801,590; 5,107,065.

Other examples of tissue-specific promoters that are used to practice this invention include those that direct expression in leaf cells following damage to the leaf (for example, from chewing insects), in tubers (for example, patatin gene promoter), and in fiber cells (an example of a developmentally-regulated fiber cell protein is E6, see, e.g., John (1992) Proc. Natl. Acad. Sci. USA 89:5769. The E6 gene is most active in fiber, although low levels of transcripts are found in leaf, ovule and flower.

In one aspect, tissue-specific promoters promote transcription only within a certain time frame of developmental stage within that tissue; see, e.g., Blazquez (1998) Plant Cell 10:791-800, characterizing the Arabidopsis LEAFY gene promoter; see also Cardon (1997) Plant J 12:367-77, describing the transcription factor SPL3, which recognizes a conserved sequence motif in the promoter region of the A. thaliana floral meristem identity gene AP1; and Mandel (1995) Plant Molecular Biology, Vol. 29, pp 995-1004, describing the meristem promoter eIF4.

Tissue specific promoters which are active throughout the life cycle of a particular tissue can be used. In one aspect, the nucleic acids of the invention are operably linked to a promoter active primarily only in cotton fiber cells. In one aspect, the nucleic acids of the invention are operably linked to a promoter active primarily during the stages of cotton fiber cell elongation, e.g., as described by Rinehart (1996) supra. The nucleic acids can be operably linked to the Fb12A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants.

Root-specific promoters may also be used to express the nucleic acids of the invention. Examples of root-specific promoters include the promoter from the alcohol dehydrogenase gene (DeLisle (1990) Int. Rev. Cytol. 123:39-60). Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285-1295, describing a leaf-specific promoter in maize); the ORF13 promoter from Agrobacterium rhizogenes (which exhibits high activity in roots, see, e.g., Hansen (1997) supra); a maize pollen specific promoter (see, e.g., Guerrero (1990) Mol. Gen. Genet. 224:161 168); a tomato promoter active during fruit ripening, senescence and abscission of leaves and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

In one aspect, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

The nucleic acids of the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- (e.g., hormone- or pesticide-) induced promoters, i.e., promoter responsive to a chemical which can be applied to the transgenic plant in the field, expression of a polypeptide of the invention can be induced at a particular stage of development or maturation of the plant or plant part (e.g., fruit or seed). Thus, the invention also provides transgenic plants comprising an inducible protein coding sequence (e.g., a gene) encoding a polypeptide of the invention; which alternative can comprise a host range in a broad or a limited range, e.g., limited to target plant species, such as corn, rice, barley, soybean, tomato, wheat, potato or other crops. In one aspect, the inducible protein coding sequence (e.g., a gene) is inducible at any stage of development or maturation of the crop, including plant parts (e.g., fruits or seeds).

One of skill will recognize that a tissue-specific plant promoter may drive expression of operably linked sequences in tissues other than the target tissue. Thus, in one aspect, a tissue-specific promoter is one that drives expression preferentially in the target tissue or cell type, but may also lead to some expression in other tissues as well.

The nucleic acids of the invention can also be operably linked to plant promoters which are inducible upon exposure to chemicals reagents. These reagents include, e.g., herbicides, synthetic auxins, or antibiotics which can be applied, e.g., sprayed, onto transgenic plants. In one aspect, inducible expression of the lignocellulosic enzyme, e.g., glycosyl hydrolase, cellulase, endoglucanase, cellobiohydrolase, beta-glucosidase, xylanase, mannanse, β-xylosidase and/or arabinofuranosidase, e.g., inducible expression of the enzyme-encoding nucleic acids of the invention, allows selection of plants with the optimal amount or timing of expression of lignocellulosic enzyme expression and/or activity. The development of plant parts can thus controlled. In this way the invention provides the means to facilitate the harvesting of plants and plant parts. For example, in various embodiments, the maize In2-2 promoter, activated by benzenesulfonamide herbicide safeners, is used (De Veylder (1997) Plant Cell Physiol. 38:568-577); application of different herbicide safeners induces distinct gene expression patterns, including expression in the root, hydathodes, and the shoot apical meristem. Coding sequences of the invention are also under the control of a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324).

In some aspects, proper polypeptide expression may require polyadenylation region at the 3′-end of the coding region. The polyadenylation region can be derived from the natural gene, from a variety of other plant (or animal or other) genes, or from genes in the Agrobacterial T-DNA.

Expression Vectors and Cloning Vehicles

The invention provides expression cassettes, expression vectors and cloning vehicles comprising nucleic acids of the invention, e.g., sequences encoding the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the invention, or antibodies of the invention.

The term “expression cassette” as used herein refers to a nucleotide sequence which is capable of affecting expression of a structural gene (i.e., a protein coding sequence, such as a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme of the invention) in a host compatible with such sequences.

Expression cassettes of the invention can comprise at least a promoter operably linked with the polypeptide coding sequence (e.g., an enzyme or antibody of the invention); and, optionally, with other sequences, e.g., transcription termination signals, signal sequence or CBH coding sequences, and the like.

Additional factors necessary or helpful in effecting expression may also be used, e.g., enhancers, alpha-factors. Thus, expression cassettes of this invention can also include (comprise, or, be contained within) plasmids, expression vectors, recombinant viruses, any form of recombinant “naked DNA” vector, artificial chromosomes, and the like.

In one aspect, a vector of the invention comprises a polypeptide coding sequence (e.g., coding sequence for an enzyme or antibody of the invention) and a nucleic acid which can infect, transfect, transiently or permanently transduce a cell. A vector of the invention can be a naked nucleic acid, or a nucleic acid complexed with protein or lipid. A vector of the invention can comprise viral or bacterial nucleic acids and/or proteins, and/or membranes (e.g., a cell membrane, a viral lipid envelope, etc.). A vector of the invention can comprise replicons (e.g., RNA replicons, bacteriophages) to which fragments of DNA may be attached and become replicated. Vectors of the invention thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids.

A recombinant microorganism or cell culture of the invention can comprise—can host—an “expression vector”, which can comprise one or both of extra-chromosomal circular and/or linear DNA and/or DNA that has been incorporated into the host chromosome(s). In one aspect, a vector is maintained by a host cell (e.g., a plant cell), and alternatively the vector is either stably replicated by the cells during mitosis as an autonomous structure, or is incorporated within the host's genome.

Expression vectors and cloning vehicles of the invention can comprise viral particles, baculovirus, phage, plasmids, phagemids, cosmids, fosmids, artificial chromosomes (e.g., yeast or bacterial artificial chromosomes), viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

Exemplary vectors include: bacterial: pQE™ vectors (Qiagen), pBLUESCRIPT™ plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention. Plasmids used to practice this invention can be commercially available, publicly available on an unrestricted basis, or can be constructed from available plasmids in accord with published procedures. Equivalent plasmids to those described herein are known in the art and will be apparent to the ordinarily skilled artisan.

The expression vector can comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

In one aspect, vectors for expressing the polypeptide or fragment thereof in eukaryotic cells contain enhancers to increase expression levels. Enhancers are cis-acting elements of DNA that can be from about 10 to about 300 by in length. They can act on a promoter to increase its transcription. Exemplary enhancers include the SV40 enhancer on the late side of the replication origin by 100 to 270, the cytomegalovirus early promoter enhancer, the polyoma enhancer on the late side of the replication origin, and the adenovirus enhancers.

A nucleic acid sequence can be inserted into a vector by a variety of procedures. In general, the sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBLUESCRIPT II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

The nucleic acids of the invention can be expressed in expression cassettes, vectors or viruses and transiently or stably expressed in plant cells and seeds. One exemplary transient expression system uses episomal expression systems, e.g., cauliflower mosaic virus (CaMV) viral RNA generated in the nucleus by transcription of an episomal mini-chromosome containing supercoiled DNA, see, e.g., Covey (1990) Proc. Natl. Acad. Sci. USA 87:1633-1637. Alternatively, coding sequences, i.e., all or sub-fragments of sequences of the invention can be inserted into a plant host cell genome becoming an integral part of the host chromosomal DNA. Sense or antisense transcripts can be expressed in this manner. A vector comprising the sequences (e.g., promoters or coding regions) from nucleic acids of the invention can comprise a marker gene that confers a selectable phenotype on a plant cell or a seed. For example, the marker may encode biocide resistance, e.g., antibiotic resistance, such as resistance to kanamycin, G418, bleomycin, hygromycin, or herbicide resistance, such as resistance to chlorosulfuron or Basta.

Expression vectors capable of expressing nucleic acids and proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

In one aspect, the expression vector can have two replication systems to allow it to be maintained in two organisms, for example in mammalian or insect cells for expression and in a prokaryotic host for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

The DNA sequence in the expression vector is operatively linked to an appropriate expression control sequence(s) (promoter) to direct RNA synthesis.

Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus and mouse metallothionein-I. Selection of the appropriate vector and promoter is well within the level of ordinary skill in the art. The expression vector also contains a ribosome binding site for translation initiation and a transcription terminator. The vector may also include appropriate sequences for amplifying expression. Promoter regions can be selected from any desired gene using chloramphenicol transferase (CAT) vectors or other vectors with selectable markers. In addition, the expression vectors in one aspect contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Mammalian expression vectors may also comprise an origin of replication, any necessary ribosome binding sites, a polyadenylation site, splice donor and acceptor sites, transcriptional termination sequences and 5′ flanking nontranscribed sequences. In some aspects, DNA sequences derived from the SV40 splice and polyadenylation sites may be used to provide the required nontranscribed genetic elements.

Vectors for expressing the polypeptide or fragment thereof in eukaryotic cells may also contain enhancers to increase expression levels. Enhancers are cis-acting elements of DNA, usually from about 10 to about 300 by in length that act on a promoter to increase its transcription. Examples include the SV40 enhancer on the late side of the replication origin by 100 to 270, the cytomegalovirus early promoter enhancer, the polyoma enhancer on the late side of the replication origin and the adenovirus enhancers.

In addition, the expression vectors can contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli and the S. cerevisiae TRP1 gene.

In some aspects, the nucleic acid encoding one of the polypeptides of the invention, or fragments comprising at least about 5, 10, 15, 20, 25, 30, 35, 40, 50, 75, 100, or 150 or more consecutive amino acids thereof is assembled in appropriate phase with a leader sequence capable of directing secretion of the translated polypeptide or fragment thereof. In one aspect, the nucleic acid can encode a fusion polypeptide in which one of the polypeptides of the invention, or fragments comprising at least 5, 10, 15, 20, 25, 30, 35, 40, 50, 75, 100, or 150 or more consecutive amino acids thereof is fused to heterologous peptides or polypeptides, such as N-terminal identification peptides which impart desired characteristics, such as increased stability or simplified purification.

The appropriate DNA sequence may be inserted into the vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley 503 Sons, Inc. 1997 and Sambrook et al., Molecular Cloning: A Laboratory Manual 2nd Ed., Cold Spring Harbor Laboratory Press (1989. Such procedures and others are deemed to be within the scope of those skilled in the art.

The vector may be, for example, in the form of a plasmid, a viral particle, or a phage. Other vectors include chromosomal, nonchromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus and pseudorabies. A variety of cloning and expression vectors for use with prokaryotic and eukaryotic hosts are described by Sambrook, et al., Molecular Cloning: A Laboratory Manual, 2nd Ed., Cold Spring Harbor, N.Y., (1989).

Host Cells and Transformed Cells

The invention also provides a transformed cell comprising a nucleic acid sequence of the invention, e.g., a sequence encoding a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme of the invention, or a vector of the invention.

The invention provides “transgenic plants” including plants or plant cells, and plant cell cultures (see, e.g., U.S. Pat. Nos. 7,045,354; 6,127,145; 5,693,506; 5,407,816) derived from those cells, including protoplasts, into which a heterologous nucleic acid sequence has been inserted, e.g., the nucleic acids and various recombinant constructs (e.g., expression cassettes) of the invention.

The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include any species of Escherichia, Salmonella, Streptomyces, Pseudomonas, Staphylococcus or Bacillus, including, e.g., Escherichia coli, Lactococcus lactis, Bacillus subtilis, Bacillus cereus, Salmonella typhimurium, Pseudomonas fluorescens. Exemplary yeast cells include any species of Pichia, Saccharomyces, Schizosaccharomyces, Kluvveromvces, Hansenula, Aspergillus or Schwanniomyces, including Pichia pastoris, Saccharomyces cerevisiae, Schizosaccharomyces pombe, Kluvveromvces lactis, Hansenula polymorpha, or filamentous fungi, e.g. Trichoderma, Aspergillus sp., including Aspergillus niger, Aspergillus phoenicis, Aspergillus carbonarius. Exemplary insect cells include any species of Spodoptera or Drosophila, including Drosophila S2 and Spodoptera Sf9. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477; U.S. Pat. No. 5,750,870.

In alternative embodiments, the polypeptides (e.g., enzymes) of this invention are used in industrial processes in a variety of forms, including cell-based systems and/or as partially or substantially purified forms, or in mixtures or other formulations, for, e.g., biofuel processing and production. In one aspect, commercial (e.g., “upscaled”) enzyme production systems are used, and this invention can use any polypeptide production system known the art, including any cell-based expression system, which include numerous strains, including any eukaryotic or prokaryotic system, including any insect, microbial, yeast, bacterial and/or fungal expression system; these alternative expression systems are well known and discussed in the literature and all are io contemplated for commercial use for producing and using the enzymes of the invention. For example, Bacillus species can be used for industrial production (see, e.g., Canadian Journal of Microbiology, 2004 Jan., 50(1):1-17). Alternatively, Streptomyces species, such as S. lividans, S. coelicolor, S. limosus, S. rimosus, S. roseosporus, and S. lividans can be used for industrial and sustainable production hosts (see, e.g., Appl Environ Microbiol. 2006 August; 72(8): 5283-5288). Aspergillus strains such as Aspergillus phoenicis, A. niger and A. carbonarius can be used to practice this invention, e.g., to produce an enzyme, such as a beta-glucosidase, of this invention (see, e.g., World Journal of Microbiology and Biotechnology, 2001, 17(5):455-461). Any Fusarium sp. can be used in an expression system to practice this invention, including e.g., Fusarium graminearum; see e.g., Royer et al. Bio/Technology 13:1479-1483 (1995). Any Aspergillus sp. can be used in an expression system to practice this invention, including e.g., A. nidulans; A. fumigatus; A. niger or A. oryzae; the genome for A. niger CBS513.88, a parent of commercially used enzyme production strains, was recently sequenced (see, e.g., Nat Biotechnol. 2007 Feb;25(2):221-31). Similarly, the genomic sequencing of Aspergillus oryzae was recently completed (Nature. 2005 Dec. 22;438(7071):1157-61). For alternative fungal expression systems that can be used to practice this invention, e.g., to express enzymes for use in industrial applications, such as biofuel production, see e.g., Advances in Fungal Biotechnology for Industry, Agriculture, and Medicine. Edited by Jan S. Tkacz & Lene Lange. 2004. Kluwer Academic & Plenum Publishers, New York; and e.g., Handbook of Industrial Mycology. Edited by Zhiqiang An. 24 Sep. 2004. Mycology Series No. 22. Marcel Dekker, New York; and e.g., Talbot (2007) “Fungal genomics goes industrial”, Nature Biotechnology 25(5):542; and in U.S. Pat. Nos. 4,885,249; 5,866,406; and international patent publication WO/2003/012071.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets can be used.

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Host cells containing the polynucleotides of interest, e.g., nucleic acids of the invention, can be cultured in conventional nutrient media modified as appropriate for activating promoters, selecting transformants or amplifying genes. The culture conditions, such as temperature, pH and the like, are those previously used with the host cell selected for expression and will be apparent to the ordinarily skilled artisan. The clones which are identified as having the specified enzyme activity may then be sequenced to identify the polynucleotide sequence encoding an enzyme having the enhanced activity.

The invention provides a method for overexpressing a recombinant the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme in a cell comprising expressing a vector comprising a nucleic acid of the invention, e.g., a nucleic acid comprising a nucleic acid sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to an exemplary sequence of the invention over a region of at least about 100 residues, wherein the sequence identities are determined by analysis with a sequence comparison algorithm or by visual inspection, or, a nucleic acid that hybridizes under stringent conditions to a nucleic acid sequence of the invention. The overexpression can be effected by any means, e.g., use of a high activity promoter, a dicistronic vector or by gene amplification of the vector.

The nucleic acids of the invention can be expressed, or overexpressed, in any in vitro or in vivo expression system. Any cell culture systems can be employed to express, or over-express, recombinant protein, including plant, bacterial, insect, yeast, fungal or mammalian cultures. Exemplary plant cell culture systems include those from rice, corn, tobacco (e.g., tobacco BY-2 cells) or any protoplast cell culture system, see, e.g., U.S. Pat. Nos. 7,045,354; 6,127,145; 5,693,506; 5,407,816.

Over-expression can be effected by appropriate choice of promoters, enhancers, vectors (e.g., use of replicon vectors, dicistronic vectors (see, e.g., Gurtu (1996) Biochem. Biophys. Res. Commun. 229:295-8), media, culture systems and the like. In one aspect, gene amplification using selection markers, e.g., glutamine synthetase (see, e.g., Sanders (1987) Dev. Biol. Stand. 66:55-63), in cell systems are used to overexpress the polypeptides of the invention. The host cell may be any of the host cells familiar to those skilled in the art, including prokaryotic cells, eukaryotic cells, mammalian cells, insect cells, or plant cells. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts (described by Gluzman, Cell, 23:175, 1981) and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Alternatively, the polypeptides of the invention, or fragments comprising at least 5, 10, 15, 20, 25, 30, 35, 40, 50, 75, 100, or 150 or more consecutive amino acids thereof can be synthetically produced by conventional peptide synthesizers, e.g., as discussed below. In other aspects, fragments or portions of the polypeptides may be employed for producing the corresponding full-length polypeptide by peptide synthesis; therefore, the fragments may be employed as intermediates for producing the full-length polypeptides.

Cell-free translation systems can also be employed to produce one of the polypeptides of the invention, or fragments comprising at least 5, 10, 15, 20, 25, 30, 35, 40, 50, 75, 100, or 150 or more consecutive amino acids thereof using mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

Amplification of Nucleic Acids

In practicing the invention, nucleic acids of the invention and nucleic acids encoding the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the invention, or modified nucleic acids of the invention, can be reproduced by amplification, e.g., PCR. Amplification can also be used to clone or modify the nucleic acids of the invention. Thus, the invention provides amplification primer sequence pairs for amplifying nucleic acids of the invention. One of skill in the art can design amplification primer sequence pairs for any part of or the full length of these sequences.

In one aspect, the invention provides a nucleic acid amplified by an amplification primer pair of the invention, e.g., a primer pair as set forth by about the first (the 5′) 12, 13, 14, 15, 16, 17, 18, 19, 20, 21, 22, 23, 24, or 25 or more residues of a nucleic acid of the invention, and about the first (the 5′) 15, 16, 17, 18, 19, 20, 21, 22, 23, 24, or 25 or more residues of the complementary strand. The invention provides amplification primer sequence pairs for amplifying a nucleic acid encoding a polypeptide having a lignocellulosic activity, e.g., a glycosyl hydrolase, cellulase, endoglucanase, beta-glucosidase, xylanase, mannanse, β-xylosidase and/or arabinofuranosidase enzyme activity, wherein the primer pair is capable of amplifying a nucleic acid comprising a sequence of the invention, or fragments or subsequences thereof. One or each member of the amplification primer sequence pair can comprise an oligonucleotide comprising at least about 10 to 50 or more consecutive bases of the sequence, or about 12, 13, 14, 15, 16, 17, 18, 19, 20, 21, 22, 23, 24, or 25 or more consecutive bases of the sequence. The invention provides amplification primer pairs, wherein the primer pair comprises a first member having a sequence as set forth by about the first (the 5′) 12, 13, 14, 15, 16, 17, 18, 19, 20, 21, 22, 23, 24, or 25 or more residues of a nucleic acid of the invention, and a second member having a sequence as set forth by about the first (the 5′) 12, 13, 14, 15, 16, 17, 18, 19, 20, 21, 22, 23, 24, or 25 or more residues of the complementary strand of the first member.

The invention provides the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes generated by amplification, e.g., polymerase chain reaction (PCR), using an amplification primer pair of the invention. The invention provides methods of making a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme by amplification, e.g., PCR, using an amplification primer pair of the invention. In one aspect, the amplification primer pair amplifies a nucleic acid from a library, e.g., a gene library, such as an environmental library.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989)

Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

### Determining Sequence Identity in Nucleic Acids and Polypeptides

The invention provides nucleic acids comprising sequences having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity (homology) to an exemplary nucleic acid of the invention (see also Tables 1 to 3, and the Sequence Listing) over a region of at least about 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550 or more, residues. The invention provides polypeptides comprising sequences having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary polypeptide of the invention (see also Tables 1 to 3, and the Sequence Listing). The extent of sequence identity (homology) may be determined using any computer program and associated parameters, including those described herein, e.g., BLASTP or BLASTN, BLAST 2.2.2. or FASTA version 3.0t78, with the default parameters.

Nucleic acid sequences of the invention can comprise at least 10, 15, 20, 25, 30, 35, 40, 50, 75, 100, 150, 200, 300, 400, or 500 or more consecutive nucleotides of an exemplary sequence of the invention and sequences substantially identical thereto. Homologous sequences and fragments of nucleic acid sequences of the invention can refer to a sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity (homology) to these sequences. Homology (sequence identity) may be determined using any of the computer programs and parameters described herein, including BLASTP or BLASTN, BLAST 2.2.2., FASTA version 3.0t78, which in alternative aspects, can use default parameters. Homologous sequences also include RNA sequences in which uridines replace the thymines in the nucleic acid sequences of the invention. The homologous sequences may be obtained using any of the procedures described herein or may result from the correction of a sequencing error. It will be appreciated that the nucleic acid sequences of the invention can be represented in the traditional single character format (See the inside back cover of Stryer, Lubert. Biochemistry, 3rd Ed., W. H Freeman & Co., New York.) or in any other format which records the identity of the nucleotides in a sequence.

In various aspects, sequence comparison (sequence identity determination) programs identified herein are used in this aspect of the invention, i.e., to determine if a nucleic acid or polypeptide sequence is within the scope of the invention. However, protein and/or nucleic acid sequence identities (homologies) may be evaluated using any sequence comparison algorithm or program known in the art. Such algorithms and programs include, but are by no means limited to, TBLASTN, BLASTP, FASTA, TFASTA and CLUSTALW (see, e.g., Pearson and Lipman, Proc. Natl. Acad. Sci. USA 85(8):2444-2448, 1988; Altschul et al., J. Mol. Biol. 215(3):403-410, 1990; Thompson Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

In one aspect, homology or sequence identity is measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology or sequence identity to various deletions, substitutions and other modifications. In one aspect, the terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. In one aspect, for sequence comparison, one sequence acts as a reference sequence, to which test sequences are compared. When using a sequence comparison algorithm, test and reference sequences are entered into a computer, subsequence coordinates are designated, if necessary and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology or sequence identity alignment algorithm of Needleman & Wunsch, J. Mol. Biol 48:443, 1970, by the search for similarity method of person & Lipman, Proc. Nat'l. Acad. Sci. USA 85:2444, 1988, by computerized implementations of these algorithms (GAP, BESTFIT, FASTA and TFASTA in the Wisconsin Genetics Software Package, Genetics Computer Group, 575 Science Dr., Madison, Wis.), or by manual alignment and visual inspection. Other algorithms for determining homology or sequence identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). At least twenty-one other genomes have already been sequenced, including, for example, M. genitalium (Fraser et al., 1995), M. jannaschii (Bult et al., 1996), H. influenzae (Fleischmann et al., 1995), E. coli (Blattner et al., 1997) and yeast (S. cerevisiae) (Mewes et al., 1997) and D. melanogaster (Adams et al., 2000). Significant progress has also been made in sequencing the genomes of model organism, such as mouse, C. elegans and Arabadopsis sp. Several databases containing genomic information annotated with some functional information are maintained by different organizations and may be accessible via the internet.

In one aspect, BLAST and BLAST 2.0 algorithms are used, which are described in, e.g., Altschul et al., Nuc. Acids Res. 25:3389-3402, 1977 and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of is either sequence is reached. The BLAST algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

The BLAST algorithm can also be used to perform a statistical analysis of the similarity between two sequences (see, e.g., Karlin & Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more in one aspect less than about 0.01 and most in one aspect less than about 0.001.

In one aspect, protein and nucleic acid sequence homologies (or sequence identities) are evaluated using the Basic Local Alignment Search Tool (“BLAST”) In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is in one aspect obtained from a protein or nucleic acid sequence database. High-scoring segment pairs are in one aspect identified (i.e., aligned) by means of a scoring matrix, many of which are known in the art. In one aspect, the scoring matrix used is the BLOSUM62 matrix (Gonnet (1992) Science 256:1443-1445; Henikoff and Henikoff (1993) Proteins 17:49-61). Less in one aspect, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). BLAST programs are accessible through the U.S. National Library of Medicine.

The parameters used with the above algorithms may be adapted depending on the sequence length and degree of homology studied. In some aspects, the parameters may be the default parameters used by the algorithms in the absence of instructions from the user.

### Computer Systems and Computer Program Products

The invention provides computers, computer systems, computer readable mediums, computer programs products and the like recorded or stored thereon the nucleic acid and polypeptide sequences of the invention. Additionally, in practicing the methods of the invention, e.g., to determine and identify sequence identities (to determine whether a nucleic acid is within the scope of the invention), structural homologies, motifs and the like in silico, a nucleic acid or polypeptide sequence of the invention can be stored, recorded, and manipulated on any medium which can be read and accessed by a computer.

As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention. As used herein, the terms “computer,” “computer program” and “processor” are used in their broadest general contexts and incorporate all such devices, as described in detail, below. A “coding sequence of or a “sequence encodes” a particular polypeptide or protein, is a nucleic acid sequence which is transcribed and translated into a polypeptide or protein when placed under the control of appropriate regulatory sequences.

The polypeptides of the invention include exemplary sequences of the invention and sequences substantially identical thereto, and subsequences (fragments) of any of the preceding sequences. In one aspect, substantially identical, or homologous, polypeptide sequences refer to a polypeptide sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity (homology) to an exemplary sequence of the invention (see also Tables 1 to 3).

Homology (sequence identity) may be determined using any of the computer programs and parameters described herein. A nucleic acid or polypeptide sequence of the invention can be stored, recorded and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid sequences of the invention, one or more of the polypeptide sequences of the invention. Another aspect of the invention is a computer readable medium having recorded thereon at least 2, 5, 10, 15, or 20 or more nucleic acid or polypeptide sequences of the invention.

Another aspect of the invention is a computer readable medium having recorded thereon one or more of the nucleic acid sequences of the invention. Another aspect of the invention is a computer readable medium having recorded thereon one or more of the polypeptide sequences of the invention. Another aspect of the invention is a computer readable medium having recorded thereon at least 2, 5, 10, 15, or 20 or more of the nucleic acid or polypeptide sequences as set forth above.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

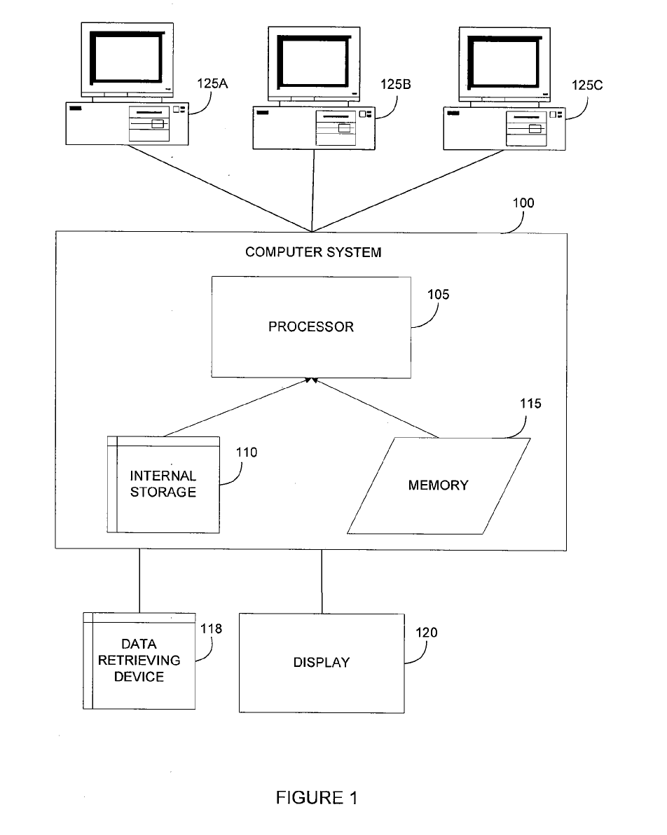

Aspects of the invention include systems (e.g., internet based systems), e.g., computer systems which store and manipulate the sequence information described herein. One example of a computer system 100 is illustrated in block diagram form in FIG. 1. As used herein, “a computer system” refers to the hardware components, software components and data storage components used to analyze a nucleotide sequence of a nucleic acid sequence of the invention, or a polypeptide sequence of the invention. In one aspect, the computer system 100 includes a processor for processing, accessing and manipulating the sequence data. The processor 105 can be any well-known type of central processing unit, such as, for example, the Pentium III from Intel Corporation, or similar processor from Sun, Motorola, Compaq, AMD or International Business Machines.

In one aspect, the computer system 100 is a general purpose system that comprises the processor 105 and one or more internal data storage components 110 for storing data and one or more data retrieving devices for retrieving the data stored on the data storage components. A skilled artisan can readily appreciate that any one of the currently available computer systems are suitable.

In one particular aspect, the computer system 100 includes a processor 105 connected to a bus which is connected to a main memory 115 (in one aspect implemented as RAM) and one or more internal data storage devices 110, such as a hard drive and/or other computer readable media having data recorded thereon. In some aspects, the computer system 100 further includes one or more data retrieving device 118 for reading the data stored on the internal data storage devices 110.

The data retrieving device 118 may represent, for example, a floppy disk drive, a compact disk drive, a magnetic tape drive, or a modem capable of connection to a remote data storage system (e.g., via the internet) etc. In some aspects, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of a nucleic acid sequence of the invention, or a polypeptide sequence of the invention, (such as search tools, compare tools and modeling tools etc.) may reside in main memory 115 during execution.

In some aspects, the computer system 100 may further comprise a sequence comparison algorithm for comparing a nucleic acid sequence of the invention, or a polypeptide sequence of the invention, stored on a computer readable medium to a reference nucleotide or polypeptide sequence(s) stored on a computer readable medium. A “sequence comparison algorithm” refers to one or more programs which are implemented (locally or remotely) on the computer system 100 to compare a nucleotide sequence with other nucleotide sequences and/or compounds stored within a data storage means. For example, the sequence comparison algorithm may compare the nucleotide sequences of a nucleic acid sequence of the invention, or a polypeptide sequence of the invention, stored on a computer readable medium to reference sequences stored on a computer readable medium to identify homologies or structural motifs.

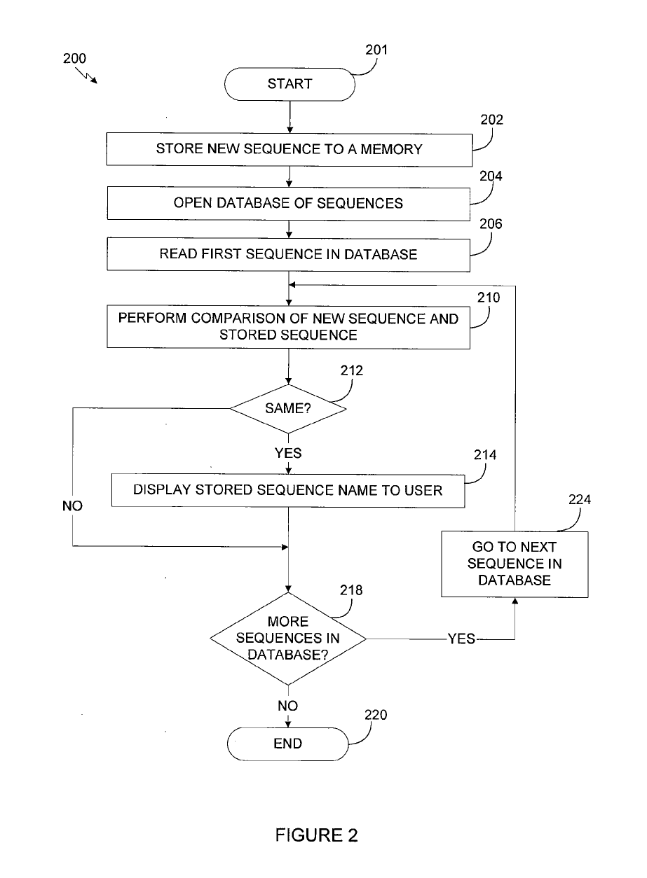

FIG. 2 is a flow diagram illustrating one aspect of a process 200 for comparing a new nucleotide or protein sequence with a database of sequences in order to determine the homology levels between the new sequence and the sequences in the database. The database of sequences can be a private database stored within the computer system 100, or a public database such as GENBANK that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the invention is a computer system comprising a processor, a data storage device having stored thereon a nucleic acid sequence of the invention, or a polypeptide sequence of the invention, a data storage device having retrievably stored thereon reference nucleotide sequences or polypeptide sequences to be compared to a nucleic acid sequence of the invention, or a polypeptide sequence of the invention and a sequence comparer for conducting the comparison. The sequence comparer may indicate a homology level between the sequences compared or identify structural motifs in the above described nucleic acid code a nucleic acid sequence of the invention, or a polypeptide sequence of the invention, or it may identify structural motifs in sequences which are compared to these nucleic acid codes and polypeptide codes. In some aspects, the data storage device may have stored thereon the sequences of at least 2, 5, 10, 15, 20, 25, 30 or 40 or more of the nucleic acid sequences of the invention, or the polypeptide sequences of the invention.

Another aspect of the invention is a method for determining the level of homology between a nucleic acid sequence of the invention, or a polypeptide sequence of the invention and a reference nucleotide sequence. The method including reading the nucleic acid code or the polypeptide code and the reference nucleotide or polypeptide sequence through the use of a computer program which determines homology levels and determining homology between the nucleic acid code or polypeptide code and the reference nucleotide or polypeptide sequence with the computer program. The computer program may be any of a number of computer programs for determining homology levels, including those specifically enumerated herein, (e.g., BLAST2N with the default parameters or with any modified parameters). The method may be implemented using the computer systems described above. The method may also be performed by reading at least 2, 5, 10, 15, 20, 25, 30 or 40 or more of the above described nucleic acid sequences of the invention, or the polypeptide sequences of the invention through use of the computer program and determining homology between the nucleic acid codes or polypeptide codes and reference nucleotide sequences or polypeptide sequences.

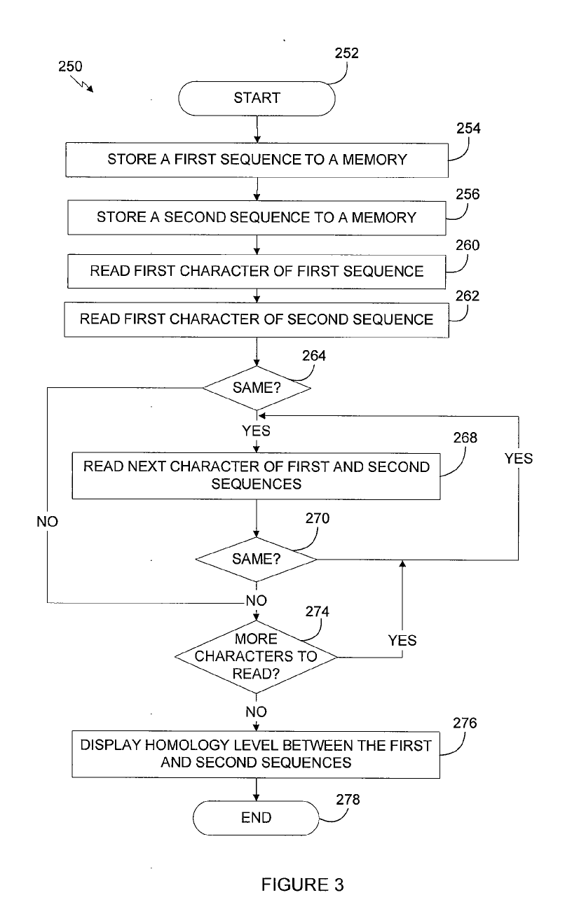

FIG. 3 is a flow diagram illustrating one aspect of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it is in one aspect in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there are not any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of a nucleic acid sequence as set forth in the invention, to one or more reference nucleotide sequences in order to determine whether the nucleic acid code of the invention, differs from a reference nucleic acid sequence at one or more positions. Optionally such a program records the length and identity of inserted, deleted or substituted nucleotides with respect to the sequence of either the reference polynucleotide or a nucleic acid sequence of the invention. In one aspect, the computer program may be a program which determines whether a nucleic acid sequence of the invention, contains a single nucleotide polymorphism (SNP) with respect to a reference nucleotide sequence.

Accordingly, another aspect of the invention is a method for determining whether a nucleic acid sequence of the invention, differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some aspects, the computer program is a program which identifies single nucleotide polymorphisms. The method may be implemented by the computer systems described above and the method illustrated in FIG. 3. The method may also be performed by reading at least 2, 5, 10, 15, 20, 25, 30, or 40 or more of the nucleic acid sequences of the invention and the reference nucleotide sequences through the use of the computer program and identifying differences between the nucleic acid codes and the reference nucleotide sequences with the computer program.

In other aspects the computer based system may further comprise an identifier for identifying features within a nucleic acid sequence of the invention or a polypeptide sequence of the invention. An “identifier” refers to one or more programs which identifies certain features within a nucleic acid sequence of the invention, or a polypeptide sequence of the invention. In one aspect, the identifier may comprise a program which identifies an open reading frame in a nucleic acid sequence of the to invention.

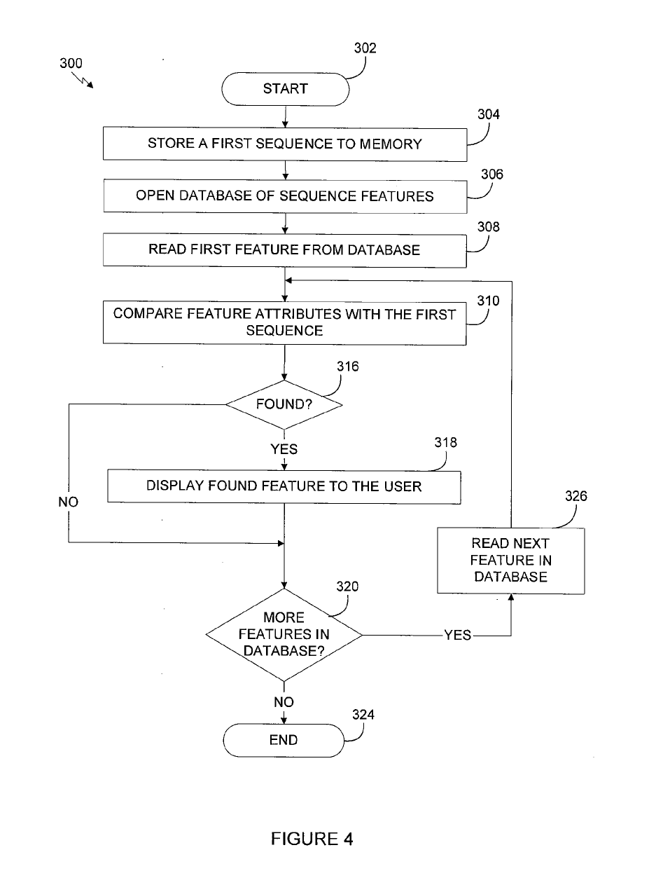

FIG. 4 is a flow diagram illustrating one aspect of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group. Alternatively, the features may be structural polypeptide motifs such as alpha helices, beta sheets, or functional polypeptide motifs such as enzymatic active sites, helix-turn-helix motifs or other motifs known to those skilled in the art.

Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user.

The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence. It should be noted, that if the feature attribute is not found in the first sequence at the decision state 316, the process 300 moves directly to the decision state 320 in order to determine if any more features exist in the database.

Accordingly, another aspect of the invention is a method of identifying a feature within a nucleic acid sequence of the invention, or a polypeptide sequence of the invention, comprising reading the nucleic acid code(s) or polypeptide code(s) through the use of a computer program which identifies features therein and identifying features within the nucleic acid code(s) with the computer program. In one aspect, computer program comprises a computer program which identifies open reading frames. The method may be performed by reading a single sequence or at least 2, 5, 10, 15, 20, 25, 30, or 40 or more of the nucleic acid sequences of the invention, or the polypeptide sequences of the invention, through the use of the computer program and identifying features within the nucleic acid codes or polypeptide codes with the computer program.

A nucleic acid sequence of the invention, or a polypeptide sequence of the invention, may be stored and manipulated in a variety of data processor programs in a variety of formats. For example, a nucleic acid sequence of the invention, or a polypeptide sequence of the invention, may be stored as text in a word processing file, such as Microsoft WORD™ or WORDPERFECT™ or as an ASCII file in a variety of database programs familiar to those of skill in the art, such as DB2™, SYBASE™, or ORACLE™. In addition, many computer programs and databases may be used as sequence comparison algorithms, identifiers, or sources of reference nucleotide sequences or polypeptide sequences to be compared to a nucleic acid sequence of the invention, or a polypeptide sequence of the invention. The following list is intended not to limit the invention but to provide guidance to programs and databases which are useful with the nucleic acid sequences of the invention, or the polypeptide sequences of the invention.

The programs and databases which may be used include, but are not limited to: MACPATTERN™ (EMBL), DISCOVERYBASE™ (Molecular Applications Group), GENEMINE™ (Molecular Applications Group), LOOK™ (Molecular Applications Group), MACLOOK™ (Molecular Applications Group), BLAST and BLAST2 (NCBI), BLASTN and BLASTX (Altschul et al, J. Mol. Biol. 215: 403, 1990), FASTA (Pearson and Lipman, Proc. Natl. Acad. Sci. USA, 85: 2444, 1988), FASTDB (Brutlag et al. Comp. App. Biosci. 6:237-245, 1990), CATALYST™ (Molecular Simulations Inc.), Catalyst/SHAPE™ (Molecular Simulations Inc.), Cerius².DBAccess™ (Molecular Simulations Inc.), HYPOGEN™ (Molecular Simulations Inc.), INSIGHT II™, (Molecular Simulations Inc.), DISCOVER™ (Molecular Simulations Inc.), CHARMm™ (Molecular Simulations Inc.), FELIX™ (Molecular Simulations Inc.), DELPHI™, (Molecular Simulations Inc.), QuanteMM™, (Molecular Simulations Inc.), Homology (Molecular Simulations Inc.), MODELER™ (Molecular Simulations Inc.), ISIS™ (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwents's World Drug Index database, the BioByteMasterFile database, the Genbank database and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices and beta sheets, signal sequences encoding signal peptides which direct the secretion of the encoded proteins, sequences implicated in transcription regulation such as homeoboxes, acidic stretches, enzymatic active sites, substrate binding sites and enzymatic cleavage sites.

### Hybridization of Nucleic Acids

The invention provides isolated, synthetic or recombinant nucleic acids that hybridize under stringent conditions to an exemplary sequence of the invention (e.g., SEQ ID NO:1, SEQ ID NO:3, etc. to SEQ ID NO:471, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, all the odd numbered SEQ ID NOs: between SEQ ID NO:489 and SEQ ID NO:700, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, and/or SEQ ID NO:720; see also Tables 1 to 3, and the Sequence Listing). The stringent conditions can be highly stringent conditions, medium stringent conditions and/or low stringent conditions, including the high and reduced stringency conditions described herein. In one aspect, it is the stringency of the wash conditions that set forth the conditions which determine whether a nucleic acid is within the scope of the invention, as discussed below.

“Hybridization” refers to the process by which a nucleic acid strand joins with a complementary strand through base pairing. Hybridization reactions can be sensitive and selective so that a particular sequence of interest can be identified even in samples in which it is present at low concentrations. Suitably stringent conditions can be defined by, for example, the concentrations of salt or formamide in the prehybridization and hybridization solutions, or by the hybridization temperature and are well known in the art. In alternative aspects, stringency can be increased by reducing the concentration of salt, increasing the concentration of formamide, or raising the hybridization temperature. In alternative aspects, nucleic acids of the invention are defined by their ability to hybridize under various stringency conditions (e.g., high, medium, and low), as set forth herein.

In one aspect, hybridization under high stringency conditions comprise about 50% formamide at about 37° C. to 42° C. In one aspect, hybridization conditions comprise reduced stringency conditions in about 35% to 25% formamide at about 30° C. to 35° C. In one aspect, hybridization conditions comprise high stringency conditions, e.g., at 42° C. in 50% formamide, 5×SSPE, 0.3% SDS and 200 ug/ml sheared and denatured salmon sperm DNA. In one aspect, hybridization conditions comprise these reduced stringency conditions, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800, 850, 900, 950, 1000, or more, residues in length. Nucleic acids shorter than full length are also included. These nucleic acids can be useful as, e.g., hybridization probes, labeling probes, PCR oligonucleotide probes, siRNA or miRNA (single or double stranded), antisense or sequences encoding antibody binding peptides (epitopes), motifs, active sites and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C.

Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 ug/ml sheared and denatured salmon sperm DNA). In one aspect, nucleic acids of the invention are defined by their ability to hybridize under reduced stringency conditions comprising 35% or 40% formamide at a reduced temperature of 35° C. or 42° C.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content) and nucleic acid type (e.g., RNA v. DNA) of the hybridizing regions of the nucleic acids can be considered in selecting hybridization conditions. An additional consideration is whether one of the nucleic acids is immobilized, for example, on a filter.

Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10× Denhardt's and 0.5 mg/ml polyriboadenylic acid. Approximately 2×10⁷ cpm (specific activity 4-9×10⁸ cpm/ug) of ³²P end-labeled oligonucleotide probe are then added to the solution. After 12-16 hours of incubation, the membrane is washed for 30 minutes at room temperature in 1× SET (150 mM NaCl, 20 mM Tris hydrochloride, pH 7.8, 1 mM Na₂EDTA) containing 0.5% SDS, followed by a 30 minute wash in fresh 1× SET at Tₘ−10° C. for the oligonucleotide probe. The membrane is then exposed to auto-radiographic film for detection of hybridization signals. All of the foregoing hybridizations would be considered to be under conditions of high stringency.

Following hybridization, a filter can be washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content) and the nucleic acid type (e.g., RNA v. DNA). Examples of progressively higher stringency condition washes are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.15M NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes. Some other examples are given below.

In one aspect, hybridization conditions comprise a wash step comprising a wash for 30 minutes at room temperature in a solution comprising 1×150 mM NaCl, 20 mM Tris hydrochloride, pH 7.8, 1 mM Na₂EDTA, 0.5% SDS, followed by a 30 minute wash in fresh solution.

Nucleic acids which have hybridized to the probe are identified by autoradiography or other conventional techniques.

The above procedures may be modified to identify nucleic acids having decreasing levels of sequence identity (homology) to the probe sequence. For example, to obtain nucleic acids of decreasing sequence identity (homology) to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

However, the selection of a hybridization format may not be critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

These methods may be used to isolate or identify nucleic acids of the invention. For example, the preceding methods may be used to isolate or identify nucleic acids having a sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity (homology) to a nucleic acid sequence selected from the group consisting of one of the sequences of the invention, or fragments comprising at least about 10, 15, 20, 25, 30, 35, 40, 50, 75, 100, 150, 200, 300, 400, or 500 consecutive bases thereof and the sequences complementary thereto. Sequence identity (homology) may be measured using the alignment algorithm. For example, the homologous polynucleotides may have a coding sequence which is a naturally occurring allelic variant of one of the coding sequences described herein. Such allelic variants may have a substitution, deletion or addition of one or more nucleotides when compared to the nucleic acids of the invention. Additionally, the above procedures may be used to isolate nucleic acids which encode polypeptides having at least about 99%, 95%, at least 90%, at least 85%, at least 80%, at least 75%, at least 70%, at least 65%, at least 60%, at least 55%, or at least 50% sequence identity (homology) to a polypeptide of the invention, or fragments comprising at least 5, 10, 15, 20, 25, 30, 35, 40, 50, 75, 100, or 150 consecutive amino acids thereof as determined using a sequence alignment algorithm (e.g., such as the FASTA version 3.0t78 algorithm with the default parameters).

### Oligonucleotides Probes and Methods for Using Them

The invention also provides nucleic acid probes that can be used, e.g., for identifying, amplifying, or isolating nucleic acids encoding a polypeptide having a lignocellulosic activity, e.g., a glycosyl hydrolase, cellulase, endoglucanase, beta-glucosidase, xylanase, mannanse, β-xylosidase and/or arabinofuranosidase enzyme activity or fragments thereof or for identifying the lignocellulosic enzyme genes. In one aspect, the probe comprises at least about 10 or more consecutive bases of a nucleic acid of the invention. Alternatively, a probe of the invention can be at least about 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 150 or about 10 to 50, about 20 to 60 about 30 to 70, consecutive bases of a sequence of a nucleic acid of the invention. The probes identify a nucleic acid by binding and/or hybridization. The probes can be used in arrays of the invention, see discussion below, including, e.g., capillary arrays. The probes of the invention can also be used to isolate other nucleic acids or polypeptides.

The isolated, synthetic or recombinant nucleic acids of the invention, the sequences complementary thereto, or a fragment comprising at least about 10, 15, 20, 25, 30, 35, 40, 50, 75, 100, 150, 200, 300, 400, or 500 consecutive bases of one of the sequences of the invention, or the sequences complementary thereto may also be used as probes to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions which permit the probe to specifically hybridize to any complementary sequences from which are present therein.

Where necessary, conditions which permit the probe to specifically hybridize to complementary sequences may be determined by placing the probe in contact with complementary sequences from samples known to contain the complementary sequence as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids.

If the sample contains the organism from which the nucleic acid was isolated, specific hybridization of the probe is then detected. Hybridization may be detected by labeling the probe with a detectable agent such as a radioactive isotope, a fluorescent dye or an enzyme capable of catalyzing the formation of a detectable product.

Many methods for using the labeled probes to detect the presence of complementary nucleic acids in a sample are familiar to those skilled in the art. These include Southern Blots, Northern Blots, colony hybridization procedures and dot blots. Protocols for each of these procedures are provided in Ausubel et al. Current Protocols in

Molecular Biology, John Wiley 503 Sons, Inc. (1997) and Sambrook et al., Molecular Cloning: A Laboratory Manual 2nd Ed., Cold Spring Harbor Laboratory Press (1989.

Alternatively, more than one probe (at least one of which is capable of specifically hybridizing to any complementary sequences which are present in the nucleic acid sample), may be used in an amplification reaction to determine whether the sample contains an organism containing a nucleic acid sequence of the invention (e.g., an organism from which the nucleic acid was isolated). In one aspect, the probes comprise oligonucleotides. In one aspect, the amplification reaction may comprise a PCR reaction. PCR protocols are described in Ausubel and Sambrook, supra. Alternatively, the amplification may comprise a ligase chain reaction, 3SR, or strand displacement reaction. (See Barany, F., “The Ligase Chain Reaction in a PCR World”, PCR Methods and Applications 1:5-16, 1991; E. Fahy et al., “Self-sustained Sequence Replication (3SR): An Isothermal Transcription-based Amplification System Alternative to PCR”, PCR Methods and Applications 1:25-33, 1991; and Walker G. T. et al., “Strand Displacement Amplification-an Isothermal in vitro DNA Amplification Technique”, Nucleic Acid Research 20:1691-1696, 1992). In such procedures, the nucleic acids in the sample are contacted with the probes, the amplification reaction is performed and any resulting amplification product is detected. The amplification product may be detected by performing gel electrophoresis on the reaction products and staining the gel with an intercalator such as ethidium bromide. Alternatively, one or more of the probes may be labeled with a radioactive isotope and the presence of a radioactive amplification product may be detected by autoradiography after gel electrophoresis.

Probes derived from sequences near the ends of the sequences of the invention, may also be used in chromosome walking procedures to identify clones containing genomic sequences located adjacent to the sequences of the invention. Such methods allow the isolation of genes which encode additional proteins from the host organism.

In one aspect, the isolated, synthetic or recombinant nucleic acids of the invention, the sequences complementary thereto, or a fragment comprising at least 10, 15, 20, 25, 30, 35, 40, 50, 75, 100, 150, 200, 300, 400, or 500 or more consecutive bases of one of the sequences of the invention, or the sequences complementary thereto are used as probes to identify and isolate related nucleic acids. In some aspects, the related nucleic acids may be cDNAs or genomic DNAs from organisms other than the one from which the nucleic acid was isolated. For example, the other organisms may be related organisms. In such procedures, a nucleic acid sample is contacted with the probe under conditions which permit the probe to specifically hybridize to related sequences. Hybridization of the probe to nucleic acids from the related organism is then detected using any of the methods described above.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tₘ, is the temperature (under defined ionic strength and pH) at which 50% of the target sequence hybridizes to a perfectly complementary probe. Very stringent conditions are selected to be equal to or about 5° C. lower than the Tₘ for a particular probe. The melting temperature of the probe may be calculated using the following formulas:

For probes between 14 and 70 nucleotides in length the melting temperature (Tₘ) is calculated using the formula: Tₘ=81.5+16.6(log [Na+])+0.41(fraction G+C)−(600/N) where N is the length of the probe.

If the hybridization is carried out in a solution containing formamide, the melting temperature may be calculated using the equation: Tₘ=81.5+16.6(log [Na+])+0.41(fraction G+C)−(0.63% formamide)−(600/N) where N is the length of the probe.

Prehybridization may be carried out in 6×SSC, 5× Denhardt's reagent, 0.5% SDS, 100 μg/ml denatured fragmented salmon sperm DNA or 6×SSC, 5× Denhardt's reagent, 0.5% SDS, 100 μg/ml denatured fragmented salmon sperm DNA, 50% formamide. The formulas for SSC and Denhardt's solutions are listed in Sambrook et al., supra.

In one aspect, hybridization is conducted by adding the detectable probe to the prehybridization solutions listed above. Where the probe comprises double stranded DNA, it is denatured before addition to the hybridization solution. In one aspect, the filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tₘ. For shorter probes, such as oligonucleotide probes, the hybridization may be conducted at 5-10° C. below the Tₘ. In one aspect, for hybridizations in 6×SSC, the hybridization is conducted at approximately 68° C. Usually, for hybridizations in 50% formamide containing solutions, the hybridization is conducted at approximately 42° C.

### Inhibiting Expression of Cellulase Enzymes

The invention provides nucleic acids complementary to (e.g., antisense sequences to) the nucleic acids of the invention, e.g., cellulase enzyme-encoding nucleic is acids, e.g., nucleic acids comprising antisense, siRNA, miRNA, ribozymes. Nucleic acids of the invention comprising antisense sequences can be capable of inhibiting the transport, splicing or transcription of cellulase enzyme-encoding genes. The inhibition can be effected through the targeting of genomic DNA or messenger RNA. The transcription or function of targeted nucleic acid can be inhibited, for example, by hybridization and/or cleavage. One exemplary set of inhibitors provided by the present invention includes oligonucleotides which are able to either bind the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme gene or message, in either case preventing or inhibiting the production or function of a lignocellulosic enzyme. The association can be through sequence specific hybridization. Another useful class of inhibitors includes oligonucleotides which cause inactivation or cleavage of the lignocellulosic enzyme message. The oligonucleotide can have enzyme activity which causes such cleavage, such as ribozymes. The oligonucleotide can be chemically modified or conjugated to an enzyme or composition capable of cleaving the complementary nucleic acid. A pool of many different such oligonucleotides can be screened for those with the desired activity. Thus, the invention provides various compositions for the inhibition of the lignocellulosic enzyme expression on a nucleic acid and/or protein level, e.g., antisense, siRNA, miRNA and ribozymes comprising the lignocellulosic enzyme sequences of the invention and the anti-cellulase, e.g., anti-endoglucanase, anti-cellobiohydrolase and/or anti-beta-glucosidase antibodies of the invention.

Inhibition of the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme expression can have a variety of industrial applications. For example, inhibition of the lignocellulosic enzyme expression can slow or prevent spoilage. In one aspect, use of compositions of the invention that inhibit the expression and/or activity of the lignocellulosic enzymes, e.g., antibodies, antisense oligonucleotides, ribozymes, siRNA and miRNA are used to slow or prevent spoilage. Thus, in one aspect, the invention provides methods and compositions comprising application onto a plant or plant product (e.g., a cereal, a grain, a fruit, seed, root, leaf, etc.) antibodies, antisense oligonucleotides, ribozymes, siRNA and miRNA of the invention to slow or prevent spoilage. These compositions also can be expressed by the plant (e.g., a transgenic plant) or another organism (e.g., a bacterium or other microorganism transformed with a lignocellulosic enzyme coding sequence, e.g., a gene, of the invention).

The compositions of the invention for the inhibition of the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme expression (e.g., antisense, iRNA, ribozymes, antibodies) can be used as pharmaceutical compositions, e.g., as anti-pathogen agents or in other therapies, e.g., as anti-microbials for, e.g., Salmonella.

Antisense Oligonucleotides

The invention provides antisense oligonucleotides capable of binding the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme message which, in one aspect, can inhibit the lignocellulosic enzyme activity by targeting mRNA. Strategies for designing antisense oligonucleotides are well described in the scientific and patent literature, and the skilled artisan can design such the lignocellulosic enzyme oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphorodithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme sequences of the invention (see, e.g., Gold (1995) J. of Biol. Chem. 270:13581-13584).

Inhibitory Ribozymes

The invention provides ribozymes capable of binding the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme message. These ribozymes can inhibit the lignocellulosic enzyme activity by, e.g., targeting mRNA. Strategies for designing ribozymes and selecting the lignocellulosic enzyme-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In one aspect, a ribozyme is a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The ribozyme of the invention, e.g., an enzymatic ribozyme RNA molecule, can be formed in a hammerhead motif, a hairpin motif, as a hepatitis delta virus motif, a group I intron motif and/or an RNaseP-like RNA in association with an RNA guide sequence. Examples of hammerhead motifs are described by, e.g., Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting. Those skilled in the art will recognize that a ribozyme of the invention, e.g., an enzymatic RNA molecule of this invention, can have a specific substrate binding site complementary to one or more of the target gene RNA regions. A ribozyme of the invention can have a nucleotide sequence within or surrounding that substrate binding site which imparts an RNA cleaving activity to the molecule.

RNA Interference (RNAi)

In one aspect, the invention provides an RNA inhibitory molecule, a so-called “RNAi” molecule, comprising a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme sequence of the invention. The RNAi molecule can comprise a double-stranded RNA (dsRNA) molecule, e.g., siRNA and/or miRNA. The RNAi molecule, e.g., siRNA and/or miRNA, can inhibit expression of a lignocellulosic enzyme gene. In one aspect, the RNAi molecule, e.g., siRNA and/or miRNA, is about 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's molecules, e.g., siRNA and/or miRNA, of the invention. The process may be practiced in vitro, ex vivo or in vivo. In one aspect, the RNAi molecules of the invention can be used to generate a loss-of-function mutation in a cell, an organ or an animal. Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

### Modification of Nucleic Acids—Making Variant Enzymes of the Invention

The invention provides methods of generating variants of the nucleic acids of the invention, e.g., those encoding a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme. These methods can be repeated or used in various combinations to generate the lignocellulosic enzymes having an altered or different activity or an altered or different stability from that of a lignocellulosic enzyme encoded by the template nucleic acid. These methods also can be repeated or used in various combinations, e.g., to generate variations in gene/ message expression, message translation or message stability. In another aspect, the genetic composition of a cell is altered by, e.g., modification of a homologous gene ex vivo, followed by its reinsertion into the cell.

A nucleic acid of the invention can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GENE SITE SATURATION MUTAGENESIS (or GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, Chromosomal Saturation Mutagenesis (CSM) and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller (1982) “Oligonucleotide-directed mutagenesis using M13-derived vectors: an efficient and general procedure for the production of point mutations in any DNA fragment” Nucleic Acids Res. 10:6487-6500; Zoller & Smith (1983)

“Oligonucleotide-directed mutagenesis of DNA fragments cloned into M13 vectors” Methods in Enzymol. 100:468-500; and Zoller (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor (1985) “The use of phosphorothioate-modified DNA in restriction enzyme reactions to prepare nicked DNA” Nucl. Acids Res. 13: 8749-8764; Taylor (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer (1988) “Improved enzymatic in vitro reactions in the gapped duplex DNA approach to oligonucleotide-directed construction of mutations” Nucl. Acids Res. 16: 7207; and Fritz (1988) “Oligonucleotide-directed construction of mutations: a gapped duplex DNA procedure without enzymatic reactions in vitro” Nucl. Acids Res. 16: 6987-6999).

Additional protocols that can be used to practice the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the α-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Protocols that can be used to practice the invention are described, e.g., in U.S. Pat. Nos. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Protocols that can be used to practice the invention (providing details regarding various diversity generating methods) are described, e.g., in U.S. patent application Ser. No. 09/407,800, “SHUFFLING OF CODON ALTERED GENES” by Patten et al. filed Sep. 28, 1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

Non-stochastic, or “directed evolution,” methods include, e.g., saturation mutagenesis, such as GENE SITE SATURATION MUTAGENESIS (or GSSM), synthetic ligation reassembly (SLR), or a combination thereof are used to modify the nucleic acids of the invention to generate the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes with new or altered properties (e.g., activity under highly acidic or alkaline conditions, high or low temperatures, and the like). Polypeptides encoded by the modified nucleic acids can be screened for an activity before testing for glucan hydrolysis or other activity. Any testing modality or protocol can be used, e.g., using a capillary array platform. See, e.g., U.S. Pat. Nos. 6,361,974; 6,280,926; 5,939,250.

GENE SITE SATURATION MUTAGENESIS or GSSM

The invention also provides methods for making enzyme using GENE SITE SATURATION MUTAGENESIS or GSSM, as described herein, and also in U.S. Pat. Nos. 6,171,820 and 6,579,258. The GENE SITE SATURATION MUTAGENESIS (or GSSM) approach is used for achieving all possible amino acid changes at each amino acid site along the polypeptide. The oligos used are comprised of a homologous sequence, a triplet sequence composed of degenerate N,N, G/T, and another homologous sequence. Thus, the degeneracy of each oligo is derived from the degeneracy of the N,N, G/T cassette contained therein. The resultant polymerization products from the use of such oligos include all possible amino acid changes at each amino acid site along the polypeptide, because the N,N, G/T sequence is able to code for all 20 amino acids. As shown, a separate degenerate oligo is used for mutagenizing each codon in a polynucleotide encoding a polypeptide.

In one aspect, codon primers containing a degenerate N,N,G/T sequence are used to introduce point mutations into a polynucleotide, e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme or an antibody of the invention, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide (e.g., the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes) molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased glucan hydrolysis activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In yet another aspect, site-saturation mutagenesis can be used together with shuffling, chimerization, recombination and other mutagenizing processes, along with screening. This invention provides for the use of any mutagenizing process(es), including saturation mutagenesis, in an iterative manner. In one exemplification, the iterative use of any mutagenizing process(es) is used in combination with screening.

The invention also provides for the use of proprietary codon primers (containing a degenerate N,N,N sequence) to introduce point mutations into a polynucleotide, so as to generate a set of progeny polypeptides in which a full range of single amino acid substitutions is represented at each amino acid position (GENE SITE SATURATION MUTAGENESIS™ (or GSSM)). The oligos used are comprised contiguously of a first homologous sequence, a degenerate N,N,N sequence and in one aspect but not necessarily a second homologous sequence. The downstream progeny translational products from the use of such oligos include all possible amino acid changes at each amino acid site along the polypeptide, because the degeneracy of the N,N,N sequence includes codons for all 20 amino acids.

In one aspect, one such degenerate oligo (comprised of one degenerate N,N,N cassette) is used for subjecting each original codon in a parental polynucleotide template to a full range of codon substitutions. In another aspect, at least two degenerate N,N,N cassettes are used—either in the same oligo or not, for subjecting at least two original codons in a parental polynucleotide template to a full range of codon substitutions. Thus, more than one N,N,N sequence can be contained in one oligo to introduce amino acid mutations at more than one site. This plurality of N,N,N sequences can be directly contiguous, or separated by one or more additional nucleotide sequence(s). In another aspect, oligos serviceable for introducing additions and deletions can be used either alone or in combination with the codons containing an N,N,N sequence, to introduce any combination or permutation of amino acid additions, deletions and/or substitutions.

In one aspect, it is possible to simultaneously mutagenize two or more io contiguous amino acid positions using an oligo that contains contiguous N,N,N triplets, i.e. a degenerate (N,N,N)ₙ sequence. In another aspect, the present invention provides for the use of degenerate cassettes having less degeneracy than the N,N,N sequence. For example, it may be desirable in some instances to use (e.g. in an oligo) a degenerate triplet sequence comprised of only one N, where the N can be in the first second or third position of the triplet. Any other bases including any combinations and permutations thereof can be used in the remaining two positions of the triplet. Alternatively, it may be desirable in some instances to use (e.g., in an oligo) a degenerate N,N,N triplet sequence, N,N,G/T, or an N,N, G/C triplet sequence.

In one aspect, use of a degenerate triplet (such as N,N,G/T or an N,N, G/C triplet sequence) is advantageous for several reasons. In one aspect, this invention provides a means to systematically and fairly easily generate the substitution of the full range of possible amino acids (for a total of 20 amino acids) into each and every amino acid position in a polypeptide. Thus, for a 100 amino acid polypeptide, the invention provides a way to systematically and fairly easily generate 2000 distinct species (i.e., 20 possible amino acids per position times 100 amino acid positions). It is appreciated that there is provided, through the use of an oligo containing a degenerate N,N,G/T or an N,N, G/C triplet sequence, 32 individual sequences that code for 20 possible amino acids. Thus, in a reaction vessel in which a parental polynucleotide sequence is subjected to saturation mutagenesis using one such oligo, there are generated 32 distinct progeny polynucleotides encoding 20 distinct polypeptides. In contrast, the use of a non-degenerate oligo in site-directed mutagenesis leads to only one progeny polypeptide product per reaction vessel.

This invention also provides for the use of nondegenerate oligos, which can optionally be used in combination with degenerate primers disclosed. It is appreciated that in some situations, it is advantageous to use nondegenerate oligos to generate specific point mutations in a working polynucleotide. This provides a means to generate specific silent point mutations, point mutations leading to corresponding amino acid changes and point mutations that cause the generation of stop codons and the corresponding expression of polypeptide fragments.

Thus, in one aspect of this invention, each saturation mutagenesis reaction vessel contains polynucleotides encoding at least 20 progeny polypeptide molecules such that all 20 amino acids are represented at the one specific amino acid position corresponding to the codon position mutagenized in the parental polynucleotide. The 32-fold degenerate progeny polypeptides generated from each saturation mutagenesis reaction vessel can be subjected to clonal amplification (e.g., cloned into a suitable E. coli host using an expression vector) and subjected to expression screening. When an individual progeny polypeptide is identified by screening to display a favorable change in property (when compared to the parental polypeptide), it can be sequenced to identify the correspondingly favorable amino acid substitution contained therein.

In one aspect, upon mutagenizing each and every amino acid position in a parental polypeptide using saturation mutagenesis as disclosed herein, a favorable amino acid changes is identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid and each of two favorable changes) and 3 positions. Thus, there are 3×3×3 or 27 total possibilities, including 7 that were previously examined—6 single point mutations (i.e., 2 at each of three positions) and no change at any position.

The invention provides for the use of saturation mutagenesis in combination with additional mutagenization processes, such as process where two or more related polynucleotides are introduced into a suitable host cell such that a hybrid polynucleotide is generated by recombination and reductive reassortment.

In addition to performing mutagenesis along the entire sequence of a gene, the instant invention provides that mutagenesis can be use to replace each of any number of bases in a polynucleotide sequence, wherein the number of bases to be mutagenized is in one aspect every integer from 15 to 100,000. Thus, instead of mutagenizing every position along a molecule, one can subject every or a discrete number of bases (in one aspect a subset totaling from 15 to 100,000) to mutagenesis. In one aspect, a separate nucleotide is used for mutagenizing each position or group of positions along a polynucleotide sequence. A group of 3 positions to be mutagenized may be a codon. The mutations can be introduced using a mutagenic primer, containing a heterologous cassette, also referred to as a mutagenic cassette. Exemplary cassettes can have from 1 to 500 bases. Each nucleotide position in such heterologous cassettes be N, A, C, G, T, A/C, A/G, A/T, C/G, C/T, G/T, C/G/T, A/G/T, A/C/T, A/C/G, or E, where E is any base that is not A, C, G, or T (E can be referred to as a designer oligo).

In one aspect, saturation mutagenesis is comprised of mutagenizing a complete io set of mutagenic cassettes (wherein each cassette is in one aspect about 1-500 bases in length) in defined polynucleotide sequence to be mutagenized (wherein the sequence to be mutagenized is in one aspect from about 15 to 100,000 bases in length). Thus, a group of mutations (ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons and groupings of particular nucleotide cassettes.

In one aspect, defined sequences to be mutagenized include a whole gene, pathway, cDNA, an entire open reading frame (ORF) and entire promoter, enhancer, repressor/transactivator, origin of replication, intron, operator, or any polynucleotide functional group. Generally, a “defined sequences” for this purpose may be any polynucleotide that a 15 base-polynucleotide sequence and polynucleotide sequences of lengths between 15 bases and 15,000 bases (this invention specifically names every integer in between). Considerations in choosing groupings of codons include types of amino acids encoded by a degenerate mutagenic cassette.

In one aspect, a grouping of mutations that can be introduced into a mutagenic cassette, this invention specifically provides for degenerate codon substitutions (using degenerate oligos) that code for 2, 3, 4, 5, 6, 7, 8, 9, 10, 11, 12, 13, 14, 15, 16, 17, 18, 19 and 20 amino acids at each position and a library of polypeptides encoded thereby.

Synthetic Ligation Reassembly (SLR)

The invention provides a non-stochastic gene modification system termed “synthetic ligation reassembly,” or simply “SLR,” a “directed evolution process,” to generate polypeptides, e.g., the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes or antibodies of the invention, with new or altered properties.

SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. Pat. Nos. 6,773,900; 6,740,506; 6,713,282; 6,635,449; 6,605,449; 6,537,776. In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are in one aspect shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more in one aspect a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated in one aspect comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

Synthetic Gene Reassembly

In one aspect, the present invention provides a non-stochastic method termed synthetic gene reassembly, that is somewhat related to stochastic shuffling, save that the nucleic acid building blocks are not shuffled or concatenated or chimerized randomly, but rather are assembled non-stochastically. See, e.g., U.S. Pat. No. 6,537,776.

The synthetic gene reassembly method does not depend on the presence of a high level of homology between polynucleotides to be shuffled. The invention can be used to non-stochastically generate libraries (or sets) of progeny molecules comprised of over 10¹⁰⁰ different chimeras. Conceivably, synthetic gene reassembly can even be used to generate libraries comprised of over 10¹⁰⁰⁰ different progeny chimeras.

Thus, in one aspect, the invention provides a non-stochastic method of producing a set of finalized chimeric nucleic acid molecules having an overall assembly order that is chosen by design, which method is comprised of the steps of generating by design a plurality of specific nucleic acid building blocks having serviceable mutually compatible ligatable ends and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, in one aspect, the overall assembly order in which the nucleic acid building blocks can be coupled is specified by the design of the ligatable ends and, if more than one assembly step is to be used, then the overall assembly order in which the nucleic acid building blocks can be coupled is also specified by the sequential order of the assembly step(s). In a one aspect of the invention, the annealed building pieces are treated with an enzyme, such as a ligase (e.g., T4 DNA ligase) to achieve covalent bonding of the building pieces.

In a another aspect, the design of nucleic acid building blocks is obtained upon analysis of the sequences of a set of progenitor nucleic acid templates that serve as a basis for producing a progeny set of finalized chimeric nucleic acid molecules. These progenitor nucleic acid templates thus serve as a source of sequence information that aids in the design of the nucleic acid building blocks that are to be mutagenized, i.e. chimerized or shuffled.

In one exemplification, the invention provides for the chimerization of a family of related genes and their encoded family of related products. In a particular exemplification, the encoded products are enzymes. The lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the present invention can be mutagenized in accordance with the methods described herein.

Thus according to one aspect of the invention, the sequences of a plurality of progenitor nucleic acid templates (e.g., polynucleotides of the invention) are aligned in order to select one or more demarcation points, which demarcation points can be located at an area of homology. The demarcation points can be used to delineate the boundaries of nucleic acid building blocks to be generated. Thus, the demarcation points identified and selected in the progenitor molecules serve as potential chimerization points in the assembly of the progeny molecules.

In one aspect, a serviceable demarcation point is an area of homology (comprised of at least one homologous nucleotide base) shared by at least two progenitor templates, but the demarcation point can be an area of homology that is shared by at least half of the progenitor templates, at least two thirds of the progenitor templates, at least three fourths of the progenitor templates and in one aspect at almost all of the progenitor templates. Even more in one aspect still a serviceable demarcation point is an area of homology that is shared by all of the progenitor templates.

In a one aspect, the gene reassembly process is performed exhaustively in order to generate an exhaustive library. In other words, all possible ordered combinations of the nucleic acid building blocks are represented in the set of finalized chimeric nucleic acid molecules. At the same time, the assembly order (i.e. the order of assembly of each building block in the 5′ to 3 sequence of each finalized chimeric nucleic acid) in each combination is by design (or non-stochastic). Because of the non-stochastic nature of the method, the possibility of unwanted side products is greatly reduced.

In another aspect, the method provides that the gene reassembly process is performed systematically, for example to generate a systematically compartmentalized library, with compartments that can be screened systematically, e.g., one by one. In other words the invention provides that, through the selective and judicious use of specific nucleic acid building blocks, coupled with the selective and judicious use of sequentially stepped assembly reactions, an experimental design can be achieved where specific sets of progeny products are made in each of several reaction vessels. This allows a systematic examination and screening procedure to be performed. Thus, it allows a potentially very large number of progeny molecules to be examined systematically in smaller groups.

Because of its ability to perform chimerizations in a manner that is highly flexible yet exhaustive and systematic as well, particularly when there is a low level of homology among the progenitor molecules, the instant invention provides for the generation of a library (or set) comprised of a large number of progeny molecules. Because of the non-stochastic nature of the instant gene reassembly invention, the progeny molecules generated in one aspect comprise a library of finalized chimeric nucleic acid molecules having an overall assembly order that is chosen by design. In a particularly aspect, such a generated library is comprised of greater than 10³ to greater than 10¹⁰⁰⁰ different progeny molecular species.

In one aspect, a set of finalized chimeric nucleic acid molecules, produced as described is comprised of a polynucleotide encoding a polypeptide. According to one aspect, this polynucleotide is a gene, which may be a man-made gene. According to another aspect, this polynucleotide is a gene pathway, which may be a man-made gene pathway. The invention provides that one or more man-made genes generated by the is invention may be incorporated into a man-made gene pathway, such as pathway operable in a eukaryotic organism (including a plant).

In another exemplification, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g., by mutagenesis) or in an in vivo process (e.g., by utilizing the gene splicing ability of a host organism). It is appreciated that in many instances the introduction of these nucleotides may also be desirable for many other reasons in addition to the potential benefit of creating a serviceable demarcation point.

Thus, according to another aspect, the invention provides that a nucleic acid building block can be used to introduce an intron. Thus, the invention provides that functional introns may be introduced into a man-made gene of the invention. The invention also provides that functional introns may be introduced into a man-made gene pathway of the invention. Accordingly, the invention provides for the generation of a chimeric polynucleotide that is a man-made gene containing one (or more) artificially introduced intron(s).

The invention also provides for the generation of a chimeric polynucleotide that is a man-made gene pathway containing one (or more) artificially introduced intron(s). In one aspect, the artificially introduced intron(s) are functional in one or more host cells for gene splicing much in the way that naturally-occurring introns serve functionally in gene splicing. The invention provides a process of producing man-made intron-containing polynucleotides to be introduced into host organisms for recombination and/or splicing.

A man-made gene produced using the invention can also serve as a substrate for recombination with another nucleic acid. Likewise, a man-made gene pathway produced using the invention can also serve as a substrate for recombination with another nucleic acid. In one aspect, the recombination is facilitated by, or occurs at, areas of homology between the man-made, intron-containing gene and a nucleic acid, which serves as a recombination partner. In one aspect, the recombination partner may also be a nucleic acid generated by the invention, including a man-made gene or a man-made gene pathway. Recombination may be facilitated by or may occur at areas of homology that exist at the one (or more) artificially introduced intron(s) in the man-made gene.

In one aspect, the synthetic gene reassembly method of the invention utilizes a plurality of nucleic acid building blocks, each of which in one aspect has two ligatable ends. The two ligatable ends on each nucleic acid building block may be two blunt ends (i.e. each having an overhang of zero nucleotides), or in one aspect one blunt end and one overhang, or more in one aspect still two overhangs. In one aspect, a useful overhang for this purpose may be a 3′ overhang or a 5′ overhang. Thus, a nucleic acid building block may have a 3′ overhang or alternatively a 5′ overhang or alternatively two 3′ overhangs or alternatively two 5′ overhangs. The overall order in which the nucleic acid building blocks are assembled to form a finalized chimeric nucleic acid molecule is determined by purposeful experimental design and is not random.

In one aspect, a nucleic acid building block is generated by chemical synthesis of two single-stranded nucleic acids (also referred to as single-stranded oligos) and contacting them so as to allow them to anneal to form a double-stranded nucleic acid building block. A double-stranded nucleic acid building block can be of variable size. The sizes of these building blocks can be small or large. Exemplary sizes for building block range from 1 base pair (not including any overhangs) to 100,000 base pairs (not including any overhangs). Other exemplary size ranges are also provided, which have lower limits of from 1 by to 10,000 by (including every integer value in between) and upper limits of from 2 by to 100,000 by (including every integer value in between).

Many methods exist by which a double-stranded nucleic acid building block can be generated that is serviceable for the invention; and these are known in the art and can be readily performed by the skilled artisan. According to one aspect, a double-stranded nucleic acid building block is generated by first generating two single stranded nucleic acids and allowing them to anneal to form a double-stranded nucleic acid building block.

The two strands of a double-stranded nucleic acid building block may be complementary at every nucleotide apart from any that form an overhang; thus containing no mismatches, apart from any overhang(s). According to another aspect, the two strands of a double-stranded nucleic acid building block are complementary at fewer than every nucleotide apart from any that form an overhang. Thus, according to this aspect, a double-stranded nucleic acid building block can be used to introduce codon degeneracy. In one aspect the codon degeneracy is introduced using the site-saturation mutagenesis described herein, using one or more N,N,G/T cassettes or alternatively using one or more N,N,N cassettes.

The in vivo recombination method of the invention can be performed blindly on a pool of unknown hybrids or alleles of a specific polynucleotide or sequence. However, it is not necessary to know the actual DNA or RNA sequence of the specific polynucleotide. The approach of using recombination within a mixed population of genes can be useful for the generation of any useful proteins, for example, a cellulase of the invention or a variant thereof. This approach may be used to generate proteins having altered specificity or activity. The approach may also be useful for the generation of hybrid nucleic acid sequences, for example, promoter regions, introns, exons, enhancer sequences, 31 untranslated regions or 51 untranslated regions of genes. Thus this approach may be used to generate genes having increased rates of expression. This approach may also be useful in the study of repetitive DNA sequences. Finally, this approach may be useful to make ribozymes or aptamers of the invention.

In one aspect the invention described herein is directed to the use of repeated cycles of reductive reassortment, recombination and selection which allow for the directed molecular evolution of highly complex linear sequences, such as DNA, RNA or proteins thorough recombination.

Optimized Directed Evolution System

The invention provides a non-stochastic gene modification system termed “optimized directed evolution system” to generate polypeptides, e.g., the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes or antibodies of the invention, with new or altered properties. In one aspect, optimized directed evolution is directed to the use of repeated cycles of reductive reassortment, recombination and selection that allow for the directed molecular evolution of nucleic acids through recombination.

Optimized directed evolution allows generation of a large population of evolved chimeric sequences, wherein the generated population is significantly enriched for sequences that have a predetermined number of crossover events. A crossover event is a point in a chimeric sequence where a shift in sequence occurs from one parental variant o to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide in one aspect includes a unique region of overlap so that mixing the oligonucleotides together results in a new variant that has each oligonucleotide fragment assembled in the correct order. Alternatively protocols for practicing these methods of the invention can be found in U.S. Pat. Nos. 6,773,900; 6,740,506; 6,713,282; 6,635,449; 6,605,449; 6,537,776; 6,361,974.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide in one aspect includes a unique region of overlap so that mixing the oligonucleotides together results in a new variant that has each oligonucleotide fragment assembled in the correct order. See also U.S. Pat. Nos. 6,773,900; 6,740,506; 6,713,282; 6,635,449; 6,605,449; 6,537,776; 6,361,974.

Determining Crossover Events

Aspects of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein is in one aspect performed in MATLAB™ (The Mathworks, Natick, Mass.) a programming language and development environment for technical computing.

Iterative Processes

Any process of the invention can be iteratively repeated, e.g., a nucleic acid encoding an altered or new cellulase phenotype, e.g., endoglucanase, cellobiohydrolase, beta-glucosidase, xylanase, mannanse, β-xylosidase and/or arabinofuranosidase enzyme of the invention, can be identified, re-isolated, again modified, re-tested for activity. This process can be iteratively repeated until a desired phenotype is engineered. For example, an entire biochemical anabolic or catabolic pathway can be engineered into a cell, including, e.g., the lignocellulosic enzyme activity.

Similarly, if it is determined that a particular oligonucleotide has no affect at all on the desired trait (e.g., a new the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

In Vivo Shuffling

In various aspects, in vivo shuffling of molecules is used in methods of the invention to provide variants of polypeptides of the invention, e.g., antibodies of the invention or cellulases of the invention, e.g., endoglucanase, cellobiohydrolase, beta-glucosidase, xylanase, mannanse, β-xylosidase and/or arabinofuranosidase enzymes, and the like. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In another aspect, the invention includes a method for producing a hybrid polynucleotide from at least a first polynucleotide and a second polynucleotide. The invention can be used to produce a hybrid polynucleotide by introducing at least a first polynucleotide and a second polynucleotide (e.g., one, or both, being an exemplary the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme-encoding sequence of the invention) which share at least one region of partial sequence homology into a suitable host cell. The regions of partial sequence homology promote processes which result in sequence reorganization producing a hybrid polynucleotide. The term “hybrid polynucleotide”, as used herein, is any nucleotide sequence which results from the method of the present invention and contains sequence from at least two original polynucleotide sequences. Such hybrid polynucleotides can result from intermolecular recombination events which promote sequence integration between DNA molecules. In addition, such hybrid polynucleotides can result from intramolecular reductive reassortment processes which utilize repeated sequences to alter a nucleotide sequence within a DNA molecule.

In one aspect, vivo reassortment focuses on “inter-molecular” processes collectively referred to as “recombination”; which in bacteria, is generally viewed as a “RecA-dependent” phenomenon. The invention can rely on recombination processes of a host cell to recombine and re-assort sequences, or the cells' ability to mediate reductive processes to decrease the complexity of quasi-repeated sequences in the cell by deletion. This process of “reductive reassortment” occurs by an “intra-molecular”, RecA-independent process.

In another aspect of the invention, novel polynucleotides can be generated by the process of reductive reassortment. The method involves the generation of constructs containing consecutive sequences (original encoding sequences), their insertion into an appropriate vector and their subsequent introduction into an appropriate host cell. The reassortment of the individual molecular identities occurs by combinatorial processes between the consecutive sequences in the construct possessing regions of homology, or between quasi-repeated units. The reassortment process recombines and/or reduces the complexity and extent of the repeated sequences and results in the production of novel molecular species. Various treatments may be applied to enhance the rate of reassortment. These could include treatment with ultra-violet light, or DNA damaging chemicals and/or the use of host cell lines displaying enhanced levels of “genetic instability”. Thus the reassortment process may involve homologous recombination or the natural property of quasi-repeated sequences to direct their own evolution.

Repeated or “quasi-repeated” sequences play a role in genetic instability. In one aspect, “quasi-repeats” are repeats that are not restricted to their original unit structure. Quasi-repeated units can be presented as an array of sequences in a construct; consecutive units of similar sequences. Once ligated, the junctions between the consecutive sequences become essentially invisible and the quasi-repetitive nature of the resulting construct is now continuous at the molecular level. The deletion process the cell performs to reduce the complexity of the resulting construct operates between the quasi-repeated sequences. The quasi-repeated units provide a practically limitless repertoire of templates upon which slippage events can occur. In one aspect, the constructs containing the quasi-repeats thus effectively provide sufficient molecular elasticity that deletion (and potentially insertion) events can occur virtually anywhere within the quasi-repetitive units.

When the quasi-repeated sequences are all ligated in the same orientation, for instance head to tail or vice versa, the cell cannot distinguish individual units. Consequently, the reductive process can occur throughout the sequences. In contrast, when for example, the units are presented head to head, rather than head to tail, the inversion delineates the endpoints of the adjacent unit so that deletion formation will favor the loss of discrete units. Thus, it is preferable with the present method that the sequences are in the same orientation. Random orientation of quasi-repeated sequences will result in the loss of reassortment efficiency, while consistent orientation of the sequences will offer the highest efficiency. However, while having fewer of the contiguous sequences in the same orientation decreases the efficiency, it may still provide sufficient elasticity for the effective recovery of novel molecules. Constructs can be made with the quasi-repeated sequences in the same orientation to allow higher efficiency.

Sequences can be assembled in a head to tail orientation using any of a variety of methods, including the following:
a) Primers that include a poly-A head and poly-T tail which when made single-stranded would provide orientation can be utilized. This is accomplished by having the first few bases of the primers made from RNA and hence easily removed RNaseH.
b) Primers that include unique restriction cleavage sites can be utilized. Multiple sites, a battery of unique sequences and repeated synthesis and ligation steps would be required.
c) The inner few bases of the primer could be thiolated and an exonuclease used to produce properly tailed molecules.

In one aspect, the recovery of the re-assorted sequences relies on the identification of cloning vectors with a reduced repetitive index (RI). The re-assorted encoding sequences can then be recovered by amplification. The products are re-cloned and expressed. The recovery of cloning vectors with reduced RI can be affected by:
1) The use of vectors only stably maintained when the construct is reduced in complexity.
2) The physical recovery of shortened vectors by physical procedures. In this case, the cloning vector would be recovered using standard plasmid isolation procedures and size fractionated on either an agarose gel, or column with a low molecular weight cut off utilizing standard procedures.
3) The recovery of vectors containing interrupted genes which can be selected when insert size decreases.
4) The use of direct selection techniques with an expression vector and the appropriate selection.

Encoding sequences (for example, genes) from related organisms may demonstrate a high degree of homology and encode quite diverse protein products. These types of sequences are particularly useful in the present invention as quasi-repeats. However, while the examples illustrated below demonstrate the reassortment of nearly identical original encoding sequences (quasi-repeats), this process is not limited to such nearly identical repeats.

The following example demonstrates an exemplary method of the invention. Encoding nucleic acid sequences (quasi-repeats) derived from three (3) unique species are described. Each sequence encodes a protein with a distinct set of properties. Each of the sequences differs by a single or a few base pairs at a unique position in the sequence. The quasi-repeated sequences are separately or collectively amplified and ligated into random assemblies such that all possible permutations and combinations are available in the population of ligated molecules. The number of quasi-repeat units can be controlled by the assembly conditions. The average number of quasi-repeated units in a construct is defined as the repetitive index (RI).

Once formed, the constructs may, or may not be size fractionated on an agarose gel according to published protocols, inserted into a cloning vector and transfected into an appropriate host cell. The cells are then propagated and “reductive reassortment” is effected. The rate of the reductive reassortment process may be stimulated by the introduction of DNA damage if desired. Whether the reduction in RI is mediated by deletion formation between repeated sequences by an “intra-molecular” mechanism, or mediated by recombination-like events through “inter-molecular” mechanisms is immaterial. The end result is a reassortment of the molecules into all possible combinations.

Optionally, the method comprises the additional step of screening the library members of the shuffled pool to identify individual shuffled library members having the ability to bind or otherwise interact, or catalyze a particular reaction (e.g., such as catalytic domain of an enzyme) with a predetermined macromolecule, such as for example a proteinaceous receptor, an oligosaccharide, virion, or other predetermined compound or structure.

The polypeptides that are identified from such libraries can be used for therapeutic, diagnostic, research and related purposes (e.g., catalysts, solutes for increasing osmolarity of an aqueous solution and the like) and/or can be subjected to one or more additional cycles of shuffling and/or selection.

In another aspect, it is envisioned that prior to or during recombination or reassortment, polynucleotides generated by the method of the invention can be subjected to agents or processes which promote the introduction of mutations into the original polynucleotides. The introduction of such mutations would increase the diversity of resulting hybrid polynucleotides and polypeptides encoded therefrom. The agents or processes which promote mutagenesis can include, but are not limited to: (+)-CC-1065, or a synthetic analog such as (+)-CC-1065-(N3-Adenine (See Sun and Hurley, (1992); an N-acetylated or deacetylated 4′-fluro-4-aminobiphenyl adduct capable of inhibiting DNA synthesis (See, for example, van de Poll et al. (1992)); or a N-acetylated or deacetylated 4-aminobiphenyl adduct capable of inhibiting DNA synthesis (See also, van de Poll et al. (1992), pp. 751-758); trivalent chromium, a trivalent chromium salt, a polycyclic aromatic hydrocarbon (PAH) DNA adduct capable of inhibiting DNA replication, such as 7-bromomethyl-benz[a]anthracene (“BMA”), tris(2,3-dibromopropyl)phosphate (“Tris-BP”), 1,2-dibromo-3-chloropropane (“DBCP”), 2-bromoacrolein (2BA), benzo[a]pyrene-7,8-dihydrodiol-9-10-epoxide (“BPDE”), a platinum(II) halogen salt, N-hydroxy-2-amino-3-methylimidazo[4,5-f]-quinoline (“N-hydroxy-IQ”) and N-hydroxy-2-amino-1-methyl-6-phenylimidazo[4,5-f]-pyridine (“N-hydroxy-PhIP”). Exemplary means for slowing or halting PCR amplification consist of UV light (+)-CC-1065 and (+)-CC-1065-(N3-Adenine). Particularly encompassed means are DNA adducts or polynucleotides comprising the DNA adducts from the polynucleotides or polynucleotides pool, which can be released or removed by a process including heating the solution comprising the polynucleotides prior to further processing.

In another aspect the invention is directed to a method of producing recombinant proteins having biological activity by treating a sample comprising double-stranded template polynucleotides encoding a wild-type protein under conditions according to the invention which provide for the production of hybrid or re-assorted polynucleotides.

Producing Sequence Variants

The invention also provides additional methods for making sequence variants of the nucleic acid (e.g., the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme) sequences of the invention. The invention also provides additional methods for isolating the lignocellulosic enzymes using the nucleic acids and polypeptides of the invention. In one aspect, the invention provides for variants of a lignocellulosic enzyme coding sequence (e.g., a gene, cDNA or message) of the invention, which can be altered by any means, including, e.g., random or stochastic methods, or, non-stochastic, or “directed evolution,” methods, as described above.

The isolated variants may be naturally occurring. Variant can also be created in vitro. Variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In one aspect of error prone PCR, the PCR is performed under conditions where the copying fidelity of the DNA polymerase is low, such that a high rate of point mutations is obtained along the entire length of the PCR product. Error prone PCR is described, e.g., in Leung (1989) Technique 1:11-15) and Caldwell (1992) PCR Methods Applic. 2:28-33. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl2, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The Is mutagenized nucleic acids are cloned into an appropriate vector and the activities of the polypeptides encoded by the mutagenized nucleic acids are evaluated.

In one aspect, variants are created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. In one aspect, clones containing the mutagenized DNA are recovered, expressed, and the activities of the polypeptide encoded therein assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

In one aspect, sexual PCR mutagenesis is an exemplary method of generating variants of the invention. In one aspect of sexual PCR mutagenesis forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/μl in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

In one aspect, variants are created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described in PCT Publication No. WO 91/16427, published Oct. 31, 1991, entitled “Methods for Phenotype Creation from Multiple Gene Populations”.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence. Recursive ensemble mutagenesis may also be used to generate variants.

Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in U.S. Pat. No. 5,965,408, filed Jul. 9, 1996, entitled, “Method of DNA Reassembly by Interrupting Synthesis” and U.S. Pat. No. 5,939,250, filed May 22, 1996, entitled, “Production of Enzymes Having Desired Activities by Mutagenesis.

The variants of the polypeptides of the invention may be variants in which one or more of the amino acid residues of the polypeptides of the sequences of the invention are substituted with a conserved or non-conserved amino acid residue (in one aspect a conserved amino acid residue); and such substituted amino acid residue may or may not be one encoded by the genetic code (e.g., the substitution may use a synthetic residue).

In one aspect, conservative substitutions are those that substitute a given amino acid in a polypeptide by another amino acid of like characteristics. In one aspect, conservative substitutions of the invention comprise the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue.

Other variants are those in which one or more of the amino acid residues of a polypeptide of the invention includes a substituent group. In one aspect, other variants are those in which the polypeptide is associated with another compound, such as a compound to increase the half-life of the polypeptide (for example, polyethylene glycol). Additional variants are those in which additional amino acids are fused to the polypeptide, such as a leader sequence, a secretory sequence, a proprotein sequence or a sequence which facilitates purification, enrichment, or stabilization of the polypeptide.

In some aspects, the fragments, derivatives and analogs retain the same biological function or activity as the polypeptides of the invention. In other aspects, the fragment, derivative, or analog includes a proprotein, such that the fragment, derivative, or analog can be activated by cleavage of the proprotein portion to produce an active polypeptide.

Optimizing Codons to Achieve High Levels of Protein Expression in Host Cells

The invention provides methods for modifying the lignocellulosic enzyme, e.g., glycosyl hydrolase, cellulase, endoglucanase, cellobiohydrolase, beta-glucosidase, xylanase, mannanse, β-xylosidase and/or arabinofuranosidase, enzyme-encoding nucleic acids to modify (e.g., optimize) codon usage. In one aspect, the invention provides methods for modifying codons in a nucleic acid encoding a lignocellulosic enzyme to increase or decrease its expression in a host cell. The invention also provides nucleic acids encoding a lignocellulosic enzyme modified to increase its expression in a host cell, the lignocellulosic enzyme so modified, and methods of making the modified the lignocellulosic enzymes. The method comprises identifying a “non-preferred” or a “less preferred” codon in the lignocellulosic enzyme-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Host cells for expressing the nucleic acids, expression cassettes and vectors of the invention include bacteria, yeast, fungi, plant cells, insect cells and mammalian cells (see discussion, above). Thus, the invention provides methods for optimizing codon usage in all of these cells, codon-altered nucleic acids and polypeptides made by the codon-altered nucleic acids. Exemplary host cells include bacteria, such as any species of Escherichia, Lactococcus, Salmonella, Streptomyces, Pseudomonas, Staphylococcus or Bacillus, including, e.g., Escherichia coli, Lactococcus lactis, Lactobacillus gasseri, Lactococcus cremoris, Bacillus subtilis, Bacillus cereus, Salmonella typhimurium, Pseudomonas fluorescens. Exemplary host cells also include eukaryotic organisms, e.g., various fungi such as yeasts, e.g. any species of Pichia, Saccharomyces, Schizosaccharomyces, Kluyveromyces, Hansenula, Aspergillus or Schwanniomyces, including Pichia pastoris, Saccharomyces cerevisiae, Schizosaccharomyces pombe, Kluvveromyces lactis, Hansenula polymorpha, or filamentous fungi, e.g. Trichoderma, Aspergillus sp., including Aspergillus niger, and mammalian cells and cell lines and insect cells and cell lines. Thus, the invention also includes nucleic acids and polypeptides optimized for expression in these organisms and species.

For example, the codons of a nucleic acid encoding a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme isolated from a bacterial cell are modified such that the nucleic acid is optimally expressed in a bacterial cell different from the bacteria from which the lignocellulosic enzyme was derived, a yeast, a fungi, a plant cell, an insect cell or a mammalian cell. Methods for optimizing codons are well known in the art, see, e.g., U.S. Pat. No. 5,795,737; Baca (2000) Int. J. Parasitol. 30:113-118; Hale (1998) Protein Expr. Purif. 12:185-188; Narum (2001) Infect. Immun. 69:7250-7253. See also Narum (2001) Infect. Immun. 69:7250-7253, describing optimizing codons in mouse systems; Outchkourov (2002) Protein Expr. Purif. 24:18-24, describing optimizing codons in yeast; Feng (2000) Biochemistry 39:15399-15409, describing optimizing codons in E. coli; Humphreys (2000) Protein Expr. Purif. 20:252-264, describing optimizing codon usage that affects secretion in E. coli.

### Transgenic Non-Human Animals

The invention provides transgenic non-human animals comprising a nucleic acid, a polypeptide (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme), an expression cassette or vector or a transfected or transformed cell of the invention. The invention also provides methods of making and using these transgenic non-human animals.

The transgenic non-human animals can be, e.g., dogs, goats, rabbits, sheep, pigs (including all swine, hogs and related animals), cows, rats and mice, comprising the nucleic acids of the invention. These animals can be used, e.g., as in vivo models to study the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme activity, or, as models to screen for agents that change the lignocellulosic enzyme activity in vivo. The coding sequences for the polypeptides to be expressed in the transgenic non-human animals can be designed to be constitutive, or, under the control of tissue-specific, developmental-specific or inducible transcriptional regulatory factors.

Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice. U.S. Pat. No. 6,187,992, describes making and using a transgenic mouse.

“Knockout animals” can also be used to practice the methods of the invention. For example, in one aspect, the transgenic or modified animals of the invention comprise a “knockout animal,” e.g., a “knockout mouse,” engineered not to express an endogenous gene, which is replaced with a gene expressing a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme of the invention, or, a fusion protein comprising a lignocellulosic enzyme of the invention.

### Transgenic Plants and Seeds

The invention provides transgenic plants and seeds (and plant parts derived therefrom, including, e.g., fruit, roots, etc.) comprising a nucleic acid, a polypeptide (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme), an expression cassette, vector, and/or a transfected or transformed cell of the invention.

The invention provides transformed, transduced, infected and transgenic plants comprising a nucleic acid of the invention, and uses these plants to practice the invention, e.g., to generate a biofuel and/or an alcohol or sugar from the plant or plant part, including whole plants, plant waste, plant by-products, plant parts (e.g., leaves, stems, flowers, roots, etc.), plant protoplasts, seeds and plant cells and progeny and cell cultures of same. In one aspect, the classes of plants used to practice this invention, including the cells and plants and methods of the invention, is as broad as the class of higher plants amenable to transformation techniques, including angiosperms (monocotyledonous (monocot) and dicotyledonous (dicot) plants), as well as gymnosperms; including plants of a variety of ploidy levels, including polyploid, diploid, haploid and hemizygous states.

The invention also provides plant products, e.g., oils, seeds, roots, leaves, extracts, fruit, pulp, pollen and the like, and/or straw or hay and the like, comprising a nucleic acid and/or a polypeptide of the invention. The transgenic plant can be dicotyledonous (a dicot) or monocotyledonous (a monocot). The invention also provides methods of making and using these transgenic plants and seeds. The transgenic plant or plant cell expressing a polypeptide of the present invention may be constructed in accordance with any method known in the art. See, for example, U.S. Pat. Nos. 6,309,872; 5,508,468, 7,151,204 and 7,157,623 (corn, or Zea mays); U.S. Pat. No. 7,141,723 (Cruciferae and Brassica plants); U.S. Pat. Nos. 6,576,820 and 6,365,807 (transgenic rice).

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme production is regulated by endogenous transcriptional or translational control elements. The invention also provides “knockout plants” where insertion of gene sequence by, e.g., homologous recombination, has disrupted the expression of the endogenous gene. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants, below.

The nucleic acids of the invention can be used to confer desired traits on essentially any plant, e.g., on starch-producing plants, such as potato, tomato, soybean, beets, corn, wheat, rice, barley, and the like, either by transient or stable expression in the plant, e.g., as a stable transgenic plant. Nucleic acids of the invention can be used to manipulate metabolic pathways of a plant in order to optimize or alter host's expression of the lignocellulosic enzyme. The can change the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme activity in a plant. Alternatively, a lignocellulosic enzyme of the invention can be used in production of a transgenic plant to produce a compound not naturally produced by that plant. This can lower production costs or create a novel product.

In one aspect, the first step in production of a transgenic plant involves making an expression construct for expression in a plant cell. These techniques are well known in the art. They can include selecting and cloning a promoter, a coding sequence for facilitating efficient binding of ribosomes to mRNA and selecting the appropriate gene terminator sequences. One exemplary constitutive promoter is CaMV35S, from the Is cauliflower mosaic virus, which generally results in a high degree of expression in plants. Other promoters are more specific and respond to cues in the plant's internal or external environment. An exemplary light-inducible promoter is the promoter from the cab gene, encoding the major chlorophyll a/b binding protein.

In one aspect, the nucleic acid is modified to achieve greater expression in a plant cell. For example, a sequence of the invention is likely to have a higher percentage of A-T nucleotide pairs compared to that seen in a plant, some of which prefer G-C nucleotide pairs. Therefore, A-T nucleotides in the coding sequence can be substituted with G-C nucleotides without significantly changing the amino acid sequence to enhance production of the gene product in plant cells.

Selectable marker gene can be added to the gene construct in order to identify plant cells or tissues that have successfully integrated the transgene. This may be necessary because achieving incorporation and expression of genes in plant cells is a rare event, occurring in just a few percent of the targeted tissues or cells. Selectable marker genes encode proteins that provide resistance to agents that are normally toxic to plants, such as antibiotics or herbicides. Only plant cells that have integrated the selectable marker gene will survive when grown on a medium containing the appropriate antibiotic or herbicide. As for other inserted genes, marker genes also require promoter and termination sequences for proper function.

In one aspect, making transgenic plants or seeds comprises incorporating sequences of the invention and, optionally, marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5, 681,730, describing particle-mediated transformation of gymnosperms.

In one aspect, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

Nucleic acids, e.g., expression constructs, can also be introduced in to plant cells using recombinant viruses. Plant cells can be transformed using viral vectors, such as, e.g., tobacco mosaic virus derived vectors (Rouwendal (1997) Plant Mol. Biol. 33:989-999), see Porta (1996) “Use of viral replicons for the expression of genes in plants,” Mol. Biotechnol. 5:209-221.

Alternatively, nucleic acids, e.g., an expression construct, can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, e.g., Horsch (1984) Science 233:496-498; Fraley (1983) Proc. Natl. Acad. Sci. USA 80:4803 (1983); Gene Transfer to Plants, Potrykus, ed. (Springer-Verlag, Berlin 1995). The DNA in an A. tumefaciens cell is contained in the bacterial chromosome as well as in another structure known as a Ti (tumor-inducing) plasmid. The Ti plasmid contains a stretch of DNA termed T-DNA (approximately 20 kb long) that is transferred to the plant cell in the infection process and a series of vir (virulence) genes that direct the infection process. A. tumefaciens can only infect a plant through wounds: when a plant root or stem is wounded it gives off certain chemical signals, in response to which, the vir genes of A. tumefaciens become activated and direct a series of events necessary for the transfer of the T-DNA from the Ti plasmid to the plant's chromosome. The T-DNA then enters the plant cell through the wound. One speculation is that the T-DNA waits until the plant DNA is being replicated or transcribed, then inserts itself into the exposed plant DNA. In order to use A. tumefaciens as a transgene vector, the tumor-inducing section of T-DNA have to be removed, while retaining the T-DNA border regions and the vir genes. The transgene is then inserted between the T-DNA border regions, where it is transferred to the plant cell and becomes integrated into the plant's chromosomes.

The invention provides for the transformation of monocotyledonous plants using the nucleic acids of the invention, including important cereals, see Hiei (1997) Plant Mol. Biol. 35:205-218. See also, e.g., Horsch, Science (1984) 233:496; Fraley (1983) Proc. Natl. Acad. Sci USA 80:4803; Thykjaer (1997) supra; Park (1996) Plant Mol. Biol. 32:1135-1148, discussing T-DNA integration into genomic DNA. See also D'Halluin, U.S. Pat. No. 5,712,135, describing a process for the stable integration of a DNA comprising a gene that is functional in a cell of a cereal, or other monocotyledonous plant.

In one aspect, the third step involves selection and regeneration of whole plants capable of transmitting the incorporated target gene to the next generation. Such regeneration techniques may use manipulation of certain phytohormones in a tissue culture growth medium. In one aspect, the method uses a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985; see also U.S. Pat. No. 7,045,354. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

In one aspect, after the expression cassette is stably incorporated in transgenic plants, it can be introduced into other plants by sexual crossing. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme) of the invention. The desired effects can be passed to future plant generations by standard propagation means.

In one aspect, the nucleic acids and polypeptides of the invention are expressed in or inserted in any plant or seed. Transgenic plants of the invention can be dicotyledonous or monocotyledonous. Examples of monocot transgenic plants of the invention are grasses, such as meadow grass (blue grass, Poa), forage grass such as festuca, lolium, temperate grass, such as Agrostis, and cereals, e.g., wheat, oats, rye, barley, rice, sorghum, and maize (corn). In one aspect, transgenic monocot plants and seeds comprising monocot seed-specific promoters are used to produce enzymes of the invention; methods of producing transgenic monocot seeds from the transgenic plants are described, e.g., in U.S. Pat. No. 7,157,629; production of proteins in plant seeds and seed-preferred regulatory sequences (e.g., seed-specific promoters) are also described, e.g., in U.S. Pat. Nos. 7,081,566; 7,081,565; 7,078,588; 6,566,585; 6,642,437; 6,410,828; 6,066,781; 5,889,189; 5,850,016.

Examples of dicot transgenic plants of the invention are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, the transgenic plants and seeds of the invention include a broad range of plants, including, but not limited to, species from the genera Anacardium, Arachis, Asparagus, Atropa, Avena, Brassica, Citrus, Citrullus, Capsicum, Carthamus, Cocos, Coffea, Cruciferae, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and/or Zea; additionally, the invention provides transformed, infected or transduced cells and cell cultures (including protoplasts) derived from any of these genera, and these cells—which comprise a nucleic acid, expression cassette (e.g., vector) and/or polypeptide of the invention, can be stably or transiently transformed, infected or transduced.

In alternative embodiments, the nucleic acids of the invention are expressed in (e.g., as transgenic) plants which contain fiber cells, including, e.g., cotton, silk cotton tree (Kapok, Ceiba pentandra), desert willow, creosote bush, winterfat, balsa, ramie, kenaf, hemp, roselle, jute, sisal abaca and flax. In alternative embodiments, the transgenic plants of the invention can be members of the genus Gossypium, including members of any Gossypium species, such as G. arboreum;. G. herbaceum, G. barbadense, and G. hirsutum.

Transgenic plants (and cells and cell cultures derived therefrom) of the invention can include Cruciferae and Brassica plants, Compositae plants such as sunflower and leguminous plants such as pea. Transgenic plants of the invention also include transgenic trees and parts therefrom, e.g., including any wood, leaf, bark, root, pulp or paper product; see, e.g., U.S. Pat. No. 7,141,422, describing transgenic Populus species. The invention also provides for transgenic plants (and cells and cell cultures derived therefrom) to be used for producing large amounts of the polypeptides (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme or antibody) of the invention. For example, see Palmgren (1997) Trends Genet. 13:348; Chong (1997) Transgenic Res. 6:289-296 (producing human milk protein beta-casein in transgenic potato plants using an auxin-inducible, bidirectional mannopine synthase (mas1′,2′) promoter with Agrobacterium tumefaciens-mediated leaf disc transformation methods).

Using known procedures, one of skill can screen for plants of the invention by detecting the increase or decrease of transgene mRNA or protein in transgenic plants. Means for detecting and quantitation of mRNAs or proteins are well known in the art.

### Polypeptides and Peptides

In one aspect, the invention provides isolated, synthetic or recombinant polypeptides having a sequence identity, or homology, e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, to an exemplary sequence of the invention (defined above), e.g., proteins having the sequence of SEQ ID NO:2, SEQ ID NO:4, etc. to SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, see also Table 1 to 3, and the Sequence Listing, and enzymatically active fragments (subsequences) thereof (having lignocellulosic enzyme activity) and/or immunologically active subsequences thereof (such as epitopes or immunogens, e.g., that can elicit—or generate—an antibody that can specifically bind to an exemplary polypeptide of this invention).

The percent sequence identity can be over the full length of the polypeptide, or, the identity can be over a region of at least about 15, 20, 25, 30, 35, 40, 45, 50, 60, 70, 80, 90, 100, 125, 150, 175, 200, 250, 300, 350, 400, 450, 500, 550, 600, 650, 700 or more residues. Polypeptides of the invention can also be shorter than the full length of exemplary polypeptides. In alternative aspects, the invention provides polypeptides (peptides, fragments) ranging in size between about 5 and the full length of a polypeptide, e.g., an enzyme, such as a polypeptide having a lignocellulolytic (lignocellulosic) activity, e.g., a ligninolytic and cellulolytic activity, including, e.g., a glycosyl hydrolase, cellulase, endoglucanase, cellobiohydrolase, beta-glucosidase, xylanase, mannanse, β-xylosidase and/or arabinofuranosidase enzyme; exemplary sizes being of about 5, 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, or more residues, e.g., contiguous residues of an exemplary the lignocellulosic enzyme of the invention. Peptides of the invention (e.g., a subsequence of an exemplary polypeptide of the invention) can be useful as, e.g., labeling probes, antigens (immunogens), toleragens, motifs, the lignocellulosic enzyme active sites (e.g., “catalytic domains”), signal sequences and/or prepro domains.

In alternative aspects, the invention provides polypeptides having lignocellulolytic (lignocellulosic) activity, e.g., a ligninolytic and cellulolytic activity; and in one embodiment enzymes of the invention, including polypeptides with glycosyl hydrolase, endoglucanase, cellobiohydrolase, beta-glucosidase (β-glucosidase), xylanase, mannanse, β-xylosidase and/or arabinofuranosidase, are members of a genus of polypeptides sharing specific structural elements, e.g., amino acid residues, that correlate with lignocellulolytic (lignocellulosic) activity. These shared structural elements can be used for the routine generation of the lignocellulosic enzymes, e.g., for the routine generation of glycosyl hydrolase, cellulase, endoglucanase, cellobiohydrolase, beta-glucosidase, xylanase, mannanse, β-xylosidase and/or arabinofuranosidase variants. These shared structural elements of the lignocellulosic enzymes of the invention can be used as guidance for the routine generation of the lignocellulosic enzyme variants within the scope of the genus of polypeptides of the invention.

Lignocellulolytic or lignocellulosic enzymes of the invention, e.g., glycosyl hydrolase, cellulase, endoglucanase, cellobiohydrolase, beta-glucosidase, xylanase, mannanse, β-xylosidase and/or arabinofuranosidase enzymes of the invention, encompass, but are not limited to, any polypeptide or enzymes capable of catalyzing the complete or partial breakdown and/or hydrolysis of cellulose (e.g., exemplary polypeptides of the invention, see also Tables 2 and 3, and Examples, below), or any modification or hydrolysis of a cellulose, a hemicellulose or a lignocellulotic material, e.g., a biomass material comprising cellulose, hemicellulose and lignin.

Polypeptides having glucose oxidase activity are also used to practice this invention, e.g., in mixtures (“ensembles” or “cocktails”) of enzymes of this invention, e.g., in practicing methods of this invention, or compositions of the invention, e.g., in supplements, nutritional aids, pellets, feeds, foods of this invention; in one aspect, this glucose oxidase can have activity classified as EC 1.1.3.4, can bind to beta-D-glucose (an isomer of the six carbon sugar, glucose) and/or can aid in breaking the sugar down into its metabolites; and one embodiment can be in a multimeric form, e.g., as a dimeric protein, which can catalyze the oxidation of beta-D-glucose into D-glucono-1,5-lactone, which can then hydrolyze to gluconic acid. Alternative embodiments of all, and any, polypeptide of this invention includes multimeric forms, e.g., dimeric forms, as homodimers and/or heterodimers. Tables 2 and 3 summarize exemplary enzymatic activities of exemplary polypeptides of the invention, for example, as indicated by these charts, in alternative aspects these exemplary polypeptides have, but are not limited to, the listed various activities.

In alternative embodiments, polypeptides of the invention having glycoside hydrolase activity (can also be called glycosidase activity) catalyze the hydrolysis of the glycosidic linkage to generate two smaller sugars, and thus are useful for hydrolyzing—or degrading—a biomass, such as cellulose and hemicellulose. Polypeptides of the invention having glycoside hydrolase activity also can be useful in anti-bacterial defense strategies, including targeting lysozymes, in antimicrobial pathogenesis mechanisms, for example, to target or counteract a viral neuraminidase (which is a glycoside hydrolase). Polypeptides of the invention having glycoside hydrolase activity also can be useful in the equivalent of a normal cellular function, such as in the trimming of mannosidases involved in N-linked glycoprotein biosynthesis. A glycoside hydrolase of the invention can be classified into EC 3.2.1 as an enzyme catalyzing the hydrolysis of O- or S-glycosides. A glycoside hydrolase of the invention can also be classified as either a retaining or an inverting enzyme; or either as an exo or an endo acting enzyme; thus, in some embodiment a glycoside hydrolase of the invention can act at the a non-reducing end or in the middle of its substrate, e.g., an oligo/polysaccharide chain.

In alternative embodiments, polypeptides of the invention having cellulase activity can be classified as having endoglucanase, endo-1,4-beta-glucanase, carboxymethyl cellulase, endo-1,4-beta-D-glucanase, beta-1,4-glucanase, and/or beta-1,4-endoglucan hydrolase activity. In alternative embodiments, cellulase activity of polypeptides of the invention comprise an endo-cellulase activity that breaks internal bonds to disrupt the crystalline structure of cellulose and expose individual cellulose polysaccharide chains; or, exo-cellulase activity that cleaves 2 to 4 units from the ends of exposed chains produced by endocellulase, resulting in the tetrasaccharides or disaccharide, such as cellobiose. In alternative embodiments, cellulase activity of polypeptides of the invention comprise exo-cellulase or cellobiohydrolase activity, including activity comprising working processively from the reducing end, and/or working processively from the non-reducing end, of a cellulose. In alternative embodiments, cellulase activity of polypeptides of the invention comprise a cellobiase or beta-glucosidase activity that hydrolyses the endo-cellulase product into individual monosaccharides. In alternative embodiments, cellulase activity of polypeptides of the invention comprise an oxidative cellulase activity that depolymerizes cellulose by radical reactions, e.g., as a cellobiose dehydrogenase. In alternative embodiments, cellulase activity of polypeptides of the invention comprise a cellulose phosphorylase activity that depolymerizes cellulose using phosphates instead of water. In one aspect, an enzyme of the invention can hydrolyze cellulose to beta-glucose. to In alternative embodiments, polypeptides of the invention can have a xylanase activity, including activity comprising hydrolyzing (degrading) a linear polysaccharide beta-1,4-xylan into a xylose; and in one aspect, thus breaking down a hemicellulose, which is a major component of the cell wall of plants.

Assays for Determining or Characterizing the Activity of an Enzyme

Assays for determining or characterizing the activity of an enzyme, such as determining cellulase, xylanase, cellobiohydrolase, β-glucosidase, β-xylosidase and/or arabinofuranosidase or related activity, e.g., to determine if a polypeptide is within the scope of the invention, are well known in the art, for example, see Thomas M. Wood, K. Mahalingeshwara Bhat, “Methods for Measuring Cellulase Activities”, Methods in Enzymology, 160, 87-111 (1988); U.S. Pat. Nos: 5,747,320; 5,795,766; 5,973,228; 6,022,725; 6,087,131; 6,127,160; 6,184,018; 6,423,524; 6,566,113; 6,921,655.

In some aspects, a polypeptide of the invention can have an alternative enzymatic activity. For example, the polypeptide can have endoglucanase/cellulase activity; xylanase activity; protease activity; etc.; in other words, enzymes of the invention can be multi-functional in that they have relaxed substrate specificities. In fact, studies shown herein demonstrate that two exemplary glucose oxidases of this invention enzymes are multi-functional in that they have relaxed substrate specificities, see discussion above.

“Amino acid” or “amino acid sequence” as used herein refer to an oligopeptide, peptide, polypeptide, or protein sequence, or to a fragment, portion, or subunit of any of these and to naturally occurring or synthetic molecules. “Amino acid” or “amino acid sequence” include an oligopeptide, peptide, polypeptide, or protein sequence, or to a fragment, portion, or subunit of any of these, and to naturally occurring or synthetic molecules. The term “polypeptide” as used herein, refers to amino acids joined to each other by peptide bonds or modified peptide bonds, i.e., peptide isosteres and may contain modified amino acids other than the 20 gene-encoded amino acids. The polypeptides may be modified by either natural processes, such as post-translational processing, or by chemical modification techniques which are well known in the art. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, glucan hydrolase processing, phosphorylation, prenylation, racemization, selenoylation, sulfation and transfer-RNA mediated addition of amino acids to protein such as arginylation. (See Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983)). The peptides and polypeptides of the invention also include all “mimetic” and “peptidomimetic” forms, as described in further detail, below.

As used herein, the term “isolated” means that the material (e.g., a protein or nucleic acid of the invention) is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment. As used herein, the term “purified” does not require absolute purity; rather, it is intended as a relative definition. Individual nucleic acids obtained from a library have been conventionally purified to electrophoretic homogeneity. The sequences obtained from these clones could not be obtained directly either from the library or from total human DNA. The purified nucleic acids of the invention have been purified from the remainder of the genomic DNA in the organism by at least 10⁴-10⁶ fold. In one aspect, the term “purified” includes nucleic acids which have been purified from the remainder of the genomic DNA or from other sequences in a library or other environment by at least one order of magnitude, e.g., in one aspect, two or three orders, or, four or five orders of magnitude.

“Recombinant” polypeptides or proteins refer to polypeptides or proteins produced by recombinant DNA techniques; i.e., produced from cells transformed by an exogenous DNA construct encoding the desired polypeptide or protein. “Synthetic” polypeptides or protein are those prepared by chemical synthesis. Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate.

The phrase “substantially identical” in the context of two nucleic acids or polypeptides, refers to two or more sequences that have, e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more nucleotide or amino acid residue (sequence) identity, when compared and aligned for maximum correspondence, as measured using one of the known sequence comparison algorithms or by visual inspection. In alternative aspects, the substantial identity exists over a region of at least about 100 or more residues and most commonly the sequences are substantially identical over at least about 150 to 200 or more residues. In some aspects, the sequences are substantially identical over the entire length of the coding regions.

Additionally a “substantially identical” amino acid sequence is a sequence that differs from a reference sequence by one or more conservative or non-conservative amino acid substitutions, deletions, or insertions. In one aspect, the substitution occurs at a site that is not the active site of the molecule, or, alternatively the substitution occurs at a site that is the active site of the molecule, provided that the polypeptide essentially retains its functional (enzymatic) properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a lignocellulosic enzyme, e.g., a glycosyl hydrolase, cellulase, endoglucanase, cellobiohydrolase, beta-glucosidase, xylanase, mannanse, β-xylosidase and/or arabinofuranosidase polypeptide, resulting in modification of the structure of the polypeptide, without significantly altering its biological activity. For example, amino- or carboxyl-terminal amino acids that are not required for the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme biological activity can be removed. Modified polypeptide sequences of the invention can be assayed for the lignocellulosic enzyme biological activity by any number of methods, including contacting the modified polypeptide sequence with a substrate and determining whether the modified polypeptide decreases the amount of specific substrate in the assay or increases the bioproducts of the enzymatic reaction of a functional the lignocellulosic enzyme polypeptide with the substrate.

“Fragments” as used herein are a portion of a naturally occurring protein which can exist in at least two different conformations. Fragments can have the same or substantially the same amino acid sequence as the naturally occurring protein. Fragments which have different three dimensional structures as the naturally occurring protein are also included. An example of this, is a “pro-form” molecule, such as a low activity proprotein that can be modified by cleavage to produce a mature enzyme with significantly higher activity.

In one aspect, the invention provides crystal (three-dimensional) structures of proteins and peptides, e.g., cellulases, of the invention; which can be made and analyzed using the routine protocols well known in the art, e.g., as described in MacKenzie (1998) Crystal structure of the family 7 endoglucanase I (Cel7B) from Humicola insolens at 2.2 A resolution and identification of the catalytic nucleophile by trapping of the covalent glycosyl-enzyme intermediate, Biochem. J. 335:409-416; Sakon (1997) Structure and mechanism of endo/exocellulase E4 from Thermomonospora fusca, Nat. Struct. Biol 4:810-818; Varrot (1999) Crystal structure of the catalytic core domain of the family 6 cellobiohydrolase H, Cel6A, from Humicola insolens, at 1.92 A resolution, Biochem. J. 337:297-304; illustrating and identifying specific structural elements as guidance for the routine generation of cellulase variants of the invention, and as guidance for identifying enzyme species within the scope of the invention.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides of the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants or members of a genus of polypeptides of the invention (e.g., having about 50% or more sequence identity to an exemplary sequence of the invention), routine experimentation will determine whether a mimetic is within the scope of the invention, i.e., that its structure and/or function is not substantially altered. Thus, in one aspect, a mimetic composition is within the scope of the invention if it has a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzymes activity.

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide of the invention can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L- naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

In one aspect, a residue, e.g., an amino acid, of a polypeptide of the invention can also be replaced by an amino acid (or peptidomimetic residue) of the opposite chirality. In one aspect, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

The invention also provides methods for modifying the polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. In one aspect, modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in is commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A™ automated peptide synthesizer. Such equipment provides ready access to the peptides of the invention, either by direct synthesis or by synthesis of a series of fragments that can be coupled using other known techniques.

The polypeptides of the invention include the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes in an active or inactive form. For example, the polypeptides of the invention include proproteins before “maturation” or processing of prepro sequences, e.g., by a proprotein-processing enzyme, such as a proprotein convertase to generate an “active” mature protein. The polypeptides of the invention include the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes inactive for other reasons, e.g., before “activation” by a post-translational processing event, e.g., an endo- or exo-peptidase or proteinase action, a phosphorylation event, an amidation, a glycosylation or a sulfation, a dimerization event, and the like. The polypeptides of the invention include all active forms, including active subsequences, e.g., catalytic domains or active sites, of the enzyme.

The invention includes immobilized the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes, anti-cellulase, e.g., anti-endoglucanase, anti-cellobiohydrolase and/or anti-beta-glucosidase antibodies and fragments thereof. The invention provides methods for inhibiting the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme activity, e.g., using dominant negative mutants or anti-cellulase, e.g., anti-endoglucanase, anti-cellobiohydrolase and/or anti-beta-glucosidase antibodies of the invention. The invention includes heterocomplexes, e.g., fusion proteins, heterodimers, etc., comprising the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the invention.

Polypeptides of the invention can have a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme activity under various conditions, e.g., extremes in pH and/or temperature, oxidizing agents, and the like. The invention provides methods leading to alternative the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme preparations with different catalytic efficiencies and stabilities, e.g., towards temperature, oxidizing agents and changing wash conditions. In one aspect, the lignocellulosic enzyme variants can be produced using techniques of site-directed mutagenesis and/or random mutagenesis. In one aspect, directed evolution can be used to produce a great variety of the lignocellulosic enzyme variants with alternative specificities and stability.

The proteins of the invention are also useful as research reagents to identify the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme modulators, e.g., activators or inhibitors of the lignocellulosic enzyme activity. Briefly, test samples (compounds, broths, extracts, and the like) are added to the lignocellulosic enzyme assays to determine their ability to inhibit substrate cleavage. Inhibitors identified in this way can be used in industry and research to reduce or prevent undesired proteolysis. As with the lignocellulosic enzyme inhibitors can be combined to increase the spectrum of activity.

The enzymes of the invention are also useful as research reagents to digest proteins or in protein sequencing. For example, the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes may be used to break polypeptides into smaller fragments for sequencing using, e.g. an automated sequencer.

The invention also provides methods of discovering new the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes using the nucleic acids, polypeptides and antibodies of the invention. In one aspect, phagemid libraries are screened for expression-based discovery of the lignocellulosic enzyme. In another aspect, lambda phage libraries are screened for expression-based discovery of the lignocellulosic enzymes. Screening of the phage or phagemid libraries can allow the detection of toxic clones; improved access to substrate; reduced need for engineering a host, by-passing the potential for any bias resulting from mass excision of the library; and, faster growth at low clone densities. Screening of phage or phagemid libraries can be in liquid phase or in solid phase. In one aspect, the invention provides screening in liquid phase. This gives a greater flexibility in assay conditions; additional substrate flexibility; higher sensitivity for weak clones; and ease of automation over solid phase screening.

The invention provides screening methods using the proteins and nucleic acids of the invention and robotic automation to enable the execution of many thousands of biocatalytic reactions and screening assays in a short period of time, e.g., per day, as well as ensuring a high level of accuracy and reproducibility (see discussion of arrays, below). As a result, a library of derivative compounds can be produced in a matter of weeks. For further teachings on modification of molecules, including small molecules, see PCT/US94/09174; U.S. Pat. No. 6,245,547.

In one aspect, polypeptides or fragments of the invention are obtained through biochemical enrichment or purification procedures. The sequence of potentially homologous polypeptides or fragments may be determined by the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme assays (see, e.g., Examples 1, 2 and 3, below), gel electrophoresis and/or microsequencing. The sequence of the prospective polypeptide or fragment of the invention can be compared to an exemplary polypeptide of the invention, or a fragment, e.g., comprising at least about 5, 10, 15, 20, 25, 30, 35, 40, 50, 75, 100, or 150 or more consecutive amino acids thereof using any of the programs described above.

Another aspect of the invention is an assay for identifying fragments or variants of the invention, which retain the enzymatic function of the polypeptides of the invention. For example the fragments or variants of said polypeptides, may be used to catalyze biochemical reactions, which indicate that the fragment or variant retains the enzymatic activity of a polypeptide of the invention. An exemplary assay for determining if fragments of variants retain the enzymatic activity of the polypeptides of the invention includes the steps of: contacting the polypeptide fragment or variant with a substrate molecule under conditions which allow the polypeptide fragment or variant to function and detecting either a decrease in the level of substrate or an increase in the level of the specific reaction product of the reaction between the polypeptide and substrate.

The present invention exploits the unique catalytic properties of enzymes. Whereas the use of biocatalysts (i.e., purified or crude enzymes, non-living or living cells) in chemical transformations normally requires the identification of a particular biocatalyst that reacts with a specific starting compound, the present invention uses selected biocatalysts and reaction conditions that are specific for functional groups that are present in many starting compounds, such as small molecules. Each biocatalyst is specific for one functional group, or several related functional groups and can react with many starting compounds containing this functional group.

In one aspect, the biocatalytic reactions produce a population of derivatives from a single starting compound. These derivatives can be subjected to another round of biocatalytic reactions to produce a second population of derivative compounds. Thousands of variations of the original small molecule or compound can be produced with each iteration of biocatalytic derivatization.

Enzymes react at specific sites of a starting compound without affecting the rest of the molecule, a process which is very difficult to achieve using traditional chemical methods. This high degree of biocatalytic specificity provides the means to identify a single active compound within the library. The library is characterized by the series of biocatalytic reactions used to produce it, a so-called “biosynthetic history”. Screening the library for biological activities and tracing the biosynthetic history identifies the specific reaction sequence producing the active compound. The reaction sequence is repeated and the structure of the synthesized compound determined. This mode of identification, unlike other synthesis and screening approaches, does not require immobilization technologies and compounds can be synthesized and tested free in solution using virtually any type of screening assay. It is important to note, that the high degree of specificity of enzyme reactions on functional groups allows for the “tracking” of specific enzymatic reactions that make up the biocatalytically produced library.

In one aspect, procedural steps are performed using robotic automation enabling the execution of many thousands of biocatalytic reactions and/or screening assays per day as well as ensuring a high level of accuracy and reproducibility. Robotic automation can also be used to screen for cellulase activity to determine if a polypeptide is within the scope of the invention. As a result, in one aspect, a library of derivative compounds can be produced in a matter of weeks which would take years to produce using “traditional” chemical or enzymatic screening methods.

In a particular aspect, the invention provides a method for modifying small molecules, comprising contacting a polypeptide encoded by a polynucleotide described herein, and/or enzymatically active subsequences (fragments) thereof, with a small molecule to produce a modified small molecule. A library of modified small molecules is tested to determine if a modified small molecule is present within the library, which exhibits a desired activity. A specific biocatalytic reaction which produces the modified small molecule of desired activity is identified by systematically eliminating each of the biocatalytic reactions used to produce a portion of the library and then testing the small molecules produced in the portion of the library for the presence or absence of the modified small molecule with the desired activity. The specific biocatalytic reactions which produce the modified small molecule of desired activity is optionally repeated. The biocatalytic reactions are conducted with a group of biocatalysts that react with distinct structural moieties found within the structure of a small molecule, each biocatalyst is specific for one structural moiety or a group of related structural moieties; and each biocatalyst reacts with many different small molecules which contain the distinct structural moiety.

Lignocellulosic Enzyme Signal Sequences Carbohydrate Binding Domains, and Prepro and Catalytic Domains

The invention provides lignocellulosic enzymes, e.g., glycosyl hydrolase, cellulase, endoglucanase, cellobiohydrolase, beta-glucosidase, xylanase, mannanse, β-xylosidase and/or arabinofuranosidase enzymes with or without homologous or heterologous signal sequence(s) (e.g., signal peptides (SPs)), prepro domains, carbohydrate binding domains and/or catalytic domains (CDs). The SPs, prepro domains and/or CDs of the invention can be isolated, synthetic or recombinant peptides or can be part of a fusion protein, e.g., as heterologous domain(s) in a chimeric protein. These enzymes can be multidomain constructions, for example, an enzyme of the invention can have one or more or multiple domains (e.g., SP, prepro domain, carbohydrate binding domains and/or catalytic domains) added to its sequence or spliced into its sequence (e.g., as a fusion (chimeric) protein) to replace its endogenous equivalent domain (e.g., endogenous SP, prepro domain, carbohydrate binding domains and/or catalytic domains). The invention provides isolated, synthetic or recombinant nucleic acids encoding these multidomain, or substituted domain enzymes, and the individual catalytic domains (CDs), carbohydrate binding domains, prepro domains and signal sequences (SPs, e.g., a peptide having a sequence comprising/consisting of amino terminal residues of a polypeptide of the invention) derived from a polypeptide of the invention.

The invention provides isolated, synthetic or recombinant signal sequences (e.g., signal peptides) consisting of or comprising the sequence of (a sequence as set forth in) residues 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, 1 to 40, 1 to 41, 1 to 42, 1 to 43, 1 to 44, 1 to 45, 1 to 46, or 1 to 47, or more, of a polypeptide of the invention, e.g., exemplary polypeptides of the invention, see also Tables 3 and 4, and the Sequence Listing.

In one aspect, the invention provides signal sequences comprising the first 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino terminal residues of a polypeptide of the invention.

For example, Tables 3 and 4, above, set forth exemplary signal (leader) sequences of the invention, e.g., as in the polypeptide having the sequence of SEQ ID NO:2, encoded, e.g., by SEQ ID NO:1, which has a signal sequence comprising (or consisting of) the amino terminal 33 residues of SEQ ID NO:2, or

MSRNIRKSSFIFSLLTIIVLIASMFLQTQTAQA

Additional exemplary signal sequences are similarly set forth in Tables 3 and 4, above; these are exemplary signal sequences, and the invention is not limited to these exemplary sequences, for example, another signal sequence for SEQ ID NO:2 may be

MSRNIRKSSFIFSLLTIIVLIASMFLQTQTAQ,
or
MSRNIRKSSFIFSLLTIIVLIASMFLQTQTA, etc.

Tables 1 to 4, and the sequence listing, also set forth other information regarding the exemplary sequences of the invention, as discussed in detail, above.

The invention includes polypeptides, including polypeptides of the invention, with or without a signal sequence (i.e., signal peptides (SPs), e.g., as described above and/or set forth in Tables 1 to 4), prepro domains, carbohydrate binding domains and/or catalytic domains (CDs). The invention includes polypeptides with heterologous signal sequences, prepro domains, carbohydrate binding domains and/or catalytic domains. For example, polypeptides of the invention include enzymes where their endogenous signal (leader) sequence, prepro domains, carbohydrate binding domains and/or catalytic domain is replaced with a heterologous functionally equivalent domain sequence for another similar enzyme or from a completely different enzyme source. The SP domain, prepro domain, carbohydrate binding domain and/or catalytic domain sequence (e.g., including a sequence of the invention used as a heterologous domain) can be located internally, or on the amino terminal or the carboxy terminal end of the protein.

In one aspect, a heterologous signal sequence used to practice this invention targets an encoded protein (e.g., an enzyme of the invention) to a vacuole, the endoplasmic reticulum, a chloroplast or a starch granule. In one aspect, a signal sequence of this invention targets an encoded protein (e.g., an enzyme of the invention) to a vacuole, the endoplasmic reticulum, a chloroplast or a starch granule.

The invention also includes isolated, synthetic or recombinant signal sequences, carbohydrate binding domains, prepro sequences and/or catalytic domains (e.g., “active sites”) comprising subsequences of enzymes of invention. The polypeptide comprising a signal sequence of the invention can be a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme of the invention or another lignocellulosic enzyme (not of this invention) or another enzyme or other polypeptide.

In one aspect, the invention provides a nucleic acid sequence(s) encoding a signal sequence, carbohydrate binding domain, prepro sequence and/or catalytic domain from a lignocellulosic enzyme of the invention operably linked to a nucleic acid sequence of a different the lignocellulosic enzyme, or, optionally, another enzyme; also, a signal sequence (SPs) carbohydrate binding domain, prepro sequence and/or catalytic domain from a non-lignocellulosic enzyme can be used.

The invention also provides isolated, synthetic or recombinant polypeptides comprising a signal sequence, carbohydrate binding domain (or module, “CBM”), prepro sequence and/or catalytic domain (active site) of the invention and one or more heterologous sequences. In one aspect, the heterologous sequences are sequences not naturally associated with an enzyme, or with the domains to which they are joined (e.g., as a multidomain fusion protein), or are endogenous domains but sequence modified and/or intramolecularly rearranged (re-positioned). The sequence to which a signal sequence, carbohydrate binding domain (CBM), prepro sequence and/or catalytic domain are not naturally associated can be internal to a heterologous sequence (e.g., enzyme), or on an amino terminal end, carboxy terminal end, and/or on both ends of the heterologous sequence (e.g., enzyme). For example, in one aspect, a heterologous or modified or re-positioned CBM, signal sequence and/or active site (e.g., an “at least one CBM”) is positioned approximate to a chimeric polypeptide of the invention's catalytic domain, CBM and/or signal sequence, e.g., wherein the at least one catalytic domain, CBM and/or signal sequence is positioned: e.g., approximate to the C-terminus of the polypeptide's catalytic domain, or, approximate to the N-terminus of the polypeptide's catalytic domain; in alternative embodiments, the term “approximate” means positioned one, 2, 3, 4, 5, 6, 7, 8, 9, 10, 11, 12, 13, 14, 15, 16, 17, 18, 19 or 20 or more residues from the catalytic domain, CBM, active site or C-terminus or N-terminus.

In one aspect, the invention provides an isolated, synthetic or recombinant polypeptide comprising (or consisting of) a polypeptide comprising a signal sequence (SP), CBM, prepro domain and/or catalytic domain (CD) of the invention with the proviso that it is not associated with any sequence to which it is naturally associated (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme sequence).

Plant Signal Sequences

Endogenous or heterologous signal sequence(s) used to practice this invention can include any plant signal sequence (signal peptide, SP) (note: any SP can be used to practice this invention, and the term SP includes an moiety that can direct or target a polypeptide, and includes SNs of viral, bacterial, mammalian or synthetic origin). Coding sequence for any signal sequence, including plant signal sequences, may be operably linked to a polynucleotide encoding the chimeric polypeptide, e.g., enzyme. For example, a polypeptide of the invention can comprise the maize γ-zein N-terminal signal sequence for targeting to the endoplasmic reticulum and secretion into the apoplast (the free diffusional space outside the plasma membrane); see, e.g., Torrent (1997) Plant Mol Biol. 34(1):139-149. As with all polypeptides of the invention, including these chimeric proteins, the invention provides nucleic acids encoding them.

Another exemplary signal sequence that can be used to practice this invention is the amino acid sequence motif SEKDEL for retaining polypeptides in the endoplasmic reticulum; see, e.g., Munro (1987) Cell 48(5):899-907. For example, in one aspect, the invention provides an enzyme of the invention comprising the N-terminal sequence from maize γ-zein operably linked to the motif SEKDEL, and nucleic acids encoding this chimeric sequence.

The invention also provides polypeptides of the invention operably linked to a waxy amyloplast targeting peptide; thus, the polypeptide will be targeted to an amyloplast or to a starch granule because of this fusion to the waxy amyloplast targeting peptide; see, e.g., Klosgen (1986), Klosgen (2001) Biochim Biophys Acta. 1541(1-2):22-33; Qbadou (2003) J. Cell Sci. 116 (Pt 5):837-846.

In another aspect, a polynucleotide encoding a hyperthermophilic processing enzyme is operably linked to a chloroplast (amyloplast) transit peptide (CTP) and a CBH in the form of a starch binding domain, e.g., from the waxy gene; see, e.g., Klosgen (1991) Mol. Gen. Genet. 225(2):297-304; Gutensohn (2006) Plant Biol. (Stuttg). 8(1):18-30; Ji (2004) Plant Biotechnol. J. 2(3):251-260. Starch binding domains are well known in the art, and any starch binding domain can be used to practice this invention, e.g., as a heterologous domain linked to or as part of (e.g., as a chimeric recombinant protein) an enzyme of this invention; see e.g., Firouzabadi Planta (2006) October 13^{th} Epub; Ji (2004) Plant Biotechnol. J. 2(3):251-260. In another aspect, an enzyme of the invention is designed to target starch granules by operably linking it to a starch binding domain, e.g., the waxy starch binding domain; this linking—as with other heterologous domains joined to an enzyme of the invention—can be as a chimeric recombinant protein or chemically joined, e.g., with a linker, or electrostatically. In one aspect, the invention provides a fusion polypeptide (a chimeric recombinant protein) comprising an N-terminal amyloplast targeting sequence, e.g., from waxy, operably linked to an α-amylase fusion polypeptide comprising a starch binding domain, e.g., the waxy starch binding domain.

Carbohydrate Binding Module(s) (CBMs)

As discussed above, in one aspect, a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme of the invention is a recombinant or a chimeric, e.g., multidomain, enzyme that comprises at least one (e.g., can include multiple) carbohydrate binding module(s) (CBMs), which can be a heterologous or endogenous carbohydrate binding modules (including modified or rearranged CBMs), wherein the carbohydrate binding module(s) (CBM) can be any known module (or “domain”), e.g., including a glycosyl hydrolase binding domain, and/or, a cellulose binding module, a lignin binding module, a xylose binding module, a mannanse binding module, a xyloglucan-specific module (see, e.g., Gunnarsson (2006) Glycobiology 16:1171-1180), a arabinofuranosidase binding module, etc.; which in alternative embodiments can be from another lignocellulosic enzyme of the invention, or not of the invention; e.g., the domain is “heterologous” to the enzyme; including modules described in, e.g., U.S. Pat. App. Pub. No. 20060257984; 20060147581; U.S. Pat. No. 7,129,069. Thus, the chimeric, e.g., multidomain, enzyme of the invention can have an endogenous carbohydrate binding module rearranged or multiplied within its own sequence, or can have “switched” or replacement carbohydrate binding modules for its own endogenous modules, or can have one or more additional carbohydrate binding modules spliced into its sequences (internal or carboxy- and/or amino-terminal).

Thus, the polypeptides of the invention can comprise any of the carbohydrate binding modules that have been assigned into three major types: A, B and C; or, the chimeric polypeptide of the invention can comprise a heterologous or modified or internally rearranged CBM comprising a CBM_{—}1, CBM_{—}2, CBM_{—}2a, CBM_{—}2b, CBM_{—}3, CBM_{—}3a, CBM_{—}3b, CBM_{—}3c, CBM_{—}4, CBM_{—}5, CBM_{—}5_{—}12, CBM_{—}6, CBM_{—}7, CBM_{—}8, CBM_{—}9, CBM_{—}10, CBM_{—}11, CBM_{—}12, CBM_{—}13, CBM_{—}14, CBM_{—}15, CBM_{—}16 or any of the CBMs from a CMB family of CBM_{—}1 to CBM_{—}48, or any combination thereof.

The chimeric, or hybrid (e.g., recombinant) enzymes of the invention can comprise one or several of any other these types as heterologous or rearranged endogenous modules: including one or any module member of the CBM_{—}1 to CBM_{—}48 families, and/or Type A modules, with a flat binding surface, bind to insoluble crystalline glucans; Type B modules, displaying a binding cleft, have affinity for free single carbohydrate chains; Type C modules, which possess a solvent-exposed binding slot, have the ability to bind mono- and disaccharides (see, e.g., Protein Engineering Design and Selection (2004) 17(3):213-221; Coutinho (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Tomme (1989) FEBS Lett. 243, 239-243; Gilkes (1988) J. Biol. Chem. 263, 10401-10407; Tomme (1995) in Enzymatic Degradation of Insoluble Polysaccharides (Saddler, J. N. & Penner, M., eds.), Cellulose-binding domains: classification and properties. pp. 142-163, American Chemical Society, Washington; Henrissat (1997) Structural and sequence-based classification of glycoside hydrolases. Curr. Op. Struct. Biol. 7:637-644; Coutinho (2003) An evolving hierarchical family classification for glycosyltransferases. J. Mol. Biol. 328:307-317; Boraston (2004) Carbohydrate-binding modules: fine-tuning polysaccharide recognition. Biochem. J. 382:769-781; thus, CBMs are well characterized in the art.

In one aspect, SPs, carbohydrate binding domains, catalytic domains and/or prepro sequences of the invention are identified using routine screening protocols, or sequence homology analysis, of lignocellulosic enzymes of the invention, or other polypeptide. For example, the effect of adding or deleting or modifying a subsequence of a polypeptide of the invention on its behavior in a protein targeting pathway, the ability to bind substrates, such as carbohydrates, e.g., cellulases or lignins, to hydrolyze, etc. will identify a novel domain of the invention (pathways by which proteins are sorted and transported to their proper cellular location are often referred to as protein targeting pathways). The signal sequences of the invention can vary in length from about 10 to 65, or more, amino acid residues. Various methods of recognition of signal sequences (SPs), carbohydrate binding domains, catalytic domains and/or prepro are known to those of skill in the art. For example, in one aspect, novel lignocellulosic enzyme signal peptides are identified by a method referred to as SignalP. SignalP uses a combined neural network which recognizes both signal peptides and their cleavage sites; e.g., as described in Nielsen (1997) “Identification of prokaryotic and eukaryotic signal peptides and prediction of their cleavage sites.” Protein Engineering 10:1-6. Methods for identifying “prepro” domain sequences and signal sequences are well known in the art, see, e.g., Van de Ven (1993) Crit. Rev. Oncog. 4(2):115-136. For example, to identify a prepro sequence, the protein is purified from the extracellular space and the N-terminal protein sequence is determined and compared to the unprocessed form. In another embodiment, the heterologous SPs comprise a yeast signal sequence. A lignocellulosic enzyme of the invention can comprise a heterologous SP and/or prepro in a vector, e.g., a pPIC series vector (Invitrogen, Carlsbad, Calif.). Example 7, below, describes exemplary routine protocols for identifying carbohydrate binding module sequences.

Hybrid (Chimeric) the Lignocellulosic Enzymes and Peptide Libraries

In one aspect, the invention provides hybrid lignocellulosic enzymes, e.g., glycosyl hydrolase, cellulase, endoglucanase, cellobiohydrolase, beta-glucosidase, xylanase, mannanse, β-xylosidase and/or arabinofuranosidase enzymes as fusion proteins, which in one aspect also comprise peptide libraries, and in one embodiment these peptide libraries comprise or consist of sequences of the invention (subsequences of enzyme of the invention). The peptide libraries of the invention can be used to isolate peptide modulators (e.g., activators or inhibitors) of targets, such as the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme substrates, receptors, co-factors, modulators and the like. The peptide libraries of the invention can be used to identify formal binding partners of targets, such as ligands, e.g., cytokines, hormones, co-factors, modulators and the like. In one aspect, the invention provides chimeric proteins comprising a signal sequence (SP), prepro domain and/or catalytic domain (CD) of the invention or a combination thereof and a heterologous sequence (see above).

In one aspect, the fusion proteins of the invention (e.g., the peptide moieties) are conformationally stabilized (relative to linear peptides) to allow a higher binding affinity for targets. The invention provides fusions of lignocellulosic enzymes of the invention and other peptides, including known and random peptides. They can be fused in such a manner that the structure of the lignocellulosic enzyme is not significantly perturbed and the peptide is metabolically or structurally conformationally stabilized. This allows the creation of a peptide library that is easily monitored both for its presence within cells and its quantity.

Amino acid sequence variants of the invention can be characterized by a predetermined nature of a desired variation, e.g., a feature that sets them apart from a naturally occurring form, e.g., an allelic or interspecies variation of a lignocellulosic enzyme sequence of the invention. In one aspect, the variants of the invention exhibit the same qualitative biological activity as the naturally occurring analogue. Alternatively, the variants can be selected for having modified characteristics. In one aspect, while the site or region for introducing an amino acid sequence variation is predetermined, the mutation per se need not be predetermined. For example, in order to optimize the performance of a mutation at a given site, random mutagenesis may be conducted at the target codon or region and the expressed the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme variants screened for the optimal combination of desired activity. Techniques for making substitution mutations at predetermined sites in DNA having a known sequence are well known, as discussed herein for example, M13 primer mutagenesis and PCR mutagenesis. Screening of the mutants can be done using, e.g., assays of glucan hydrolysis. In alternative aspects, amino acid substitutions can be single residues; insertions can be on the order of from about 1 to 20 amino acids, although considerably larger insertions can be done. Deletions can range from about 1 to about 20, 30, 40, 50, 60, 70 residues or more. To obtain a final derivative with the optimal properties, substitutions, deletions, insertions or any combination thereof may be used. Generally, these changes are done on a few amino acids to minimize the alteration of the molecule. However, larger changes may be tolerated in certain circumstances.

The invention provides the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes where the structure of the polypeptide backbone, the secondary or the tertiary structure, e.g., an alpha-helical or beta-sheet structure, has been modified. In one aspect, the charge or hydrophobicity has been modified. In one aspect, the bulk of a side chain has been modified. Substantial changes in function or immunological identity are made by selecting substitutions that are less conservative. For example, substitutions can be made which more significantly affect: the structure of the polypeptide backbone in the area of the alteration, for example a alpha-helical or a beta-sheet structure; a charge or a hydrophobic site of the molecule, which can be at an active site; or a side chain. The invention provides substitutions in polypeptide of the invention where (a) a hydrophilic residues, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. The variants can exhibit the same qualitative biological activity (i.e., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme activity) although variants can be selected to modify the characteristics of the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes as needed.

In one aspect, the lignocellulosic enzymes of the invention comprise epitopes or purification tags, signal sequences (SPs) or other fusion sequences, etc. In one aspect, the lignocellulosic enzyme of the invention can be fused to a random peptide to form a fusion polypeptide. By “fused” or “operably linked” herein is meant that the random peptide and the lignocellulosic enzyme are linked together, in such a manner as to minimize the disruption to the stability of the lignocellulosic enzyme structure, e.g., it retains the lignocellulosic enzyme activity. The fusion polypeptide (or fusion polynucleotide encoding the fusion polypeptide) can comprise further components as well, including multiple peptides at multiple loops.

In one aspect, the peptides and nucleic acids encoding them are randomized, either fully randomized or they are biased in their randomization, e.g. in nucleotide/residue frequency generally or per position. “Randomized” means that each nucleic acid and peptide consists of essentially random nucleotides and amino acids, respectively. In one aspect, the nucleic acids which give rise to the peptides can be chemically synthesized, and thus may incorporate any nucleotide at any position. Thus, when the nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized nucleic acids. The library can provide a sufficiently structurally diverse population of randomized expression products to affect a probabilistically sufficient range of cellular responses to provide one or more cells exhibiting a desired response. Thus, the invention provides an interaction library large enough so that at least one of its members will have a structure that gives it affinity for some molecule, protein, or other factor.

The invention provides a methods and sequences for generating chimeric polypeptides which may encode biologically active hybrid polypeptides (e.g., hybrid the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes). In one aspect, the original polynucleotides (e.g., an exemplary nucleic acid of the invention) encode biologically active polypeptides. In one aspect, a method of the invention produces new hybrid polypeptides by utilizing cellular processes which integrate the sequence of the original polynucleotides such that the resulting hybrid polynucleotide encodes a polypeptide demonstrating activities derived, but different, from the original biologically active polypeptides (e.g., enzyme or antibody of the invention). For example, the original polynucleotides may encode a particular enzyme (e.g., a lignocellulosic enzyme) from or found in different microorganisms. An enzyme encoded by a first polynucleotide from one organism or variant may, for example, function effectively under a particular environmental condition, e.g. high salinity. An enzyme encoded by a second polynucleotide from a different organism or variant may function effectively under a different environmental condition, such as extremely high temperatures. A hybrid polynucleotide containing sequences from the first and second original polynucleotides may encode an enzyme which exhibits characteristics of both enzymes encoded by the original polynucleotides. Thus, the enzyme encoded by the hybrid polynucleotide of the invention may function effectively under environmental conditions shared by each of the enzymes encoded by the first and second polynucleotides, e.g., high salinity and extreme temperatures.

In one aspect, a hybrid polypeptide generated by a method of the invention may exhibit specialized enzyme activity not displayed in the original enzymes. For example, following recombination and/or reductive reassortment of polynucleotides encoding the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes, the resulting hybrid polypeptide encoded by a hybrid polynucleotide can be screened for specialized non-lignocellulosic enzyme activity, e.g., screened for peptidase, phosphorylase, amidase, phosphorylase, etc., activities, obtained from each of the original enzymes. In one aspect, the hybrid polypeptide is screened to ascertain those chemical functionalities which distinguish the hybrid polypeptide from the original parent polypeptides, such as the temperature, pH or salt concentration at which the hybrid polypeptide functions.

In one aspect, the invention relates to a method for producing a biologically active hybrid polypeptide and screening such a polypeptide for enhanced activity by:
1) introducing at least a first polynucleotide in operable linkage and a second polynucleotide in operable linkage, the at least first polynucleotide and second polynucleotide sharing at least one region of partial sequence homology, into a suitable host cell;
2) growing the host cell under conditions which promote sequence reorganization resulting in a hybrid polynucleotide in operable linkage;
3) expressing a hybrid polypeptide encoded by the hybrid polynucleotide;
4) screening the hybrid polypeptide under conditions which promote identification of enhanced biological activity; and
5) isolating the a polynucleotide encoding the hybrid polypeptide.

### Isolating and Discovering Lignocellulosic Enzymes

The invention provides methods for isolating and discovering lignocellulosic enzymes and the nucleic acids that encode them. Polynucleotides or enzymes may be isolated from individual organisms (“isolates”), collections of organisms that have been grown in defined media (“enrichment cultures”), or, uncultivated organisms (“environmental samples”). The organisms can be isolated by, e.g., in vivo biopanning (see discussion, below). The use of a culture-independent approach to derive polynucleotides encoding novel bioactivities from environmental samples is most preferable since it allows one to access untapped resources of biodiversity. Polynucleotides or enzymes also can be isolated from any one of numerous organisms, e.g. bacteria. In addition to whole cells, polynucleotides or enzymes also can be isolated from crude enzyme preparations derived from cultures of these organisms, e.g., bacteria.

In one aspect, “environmental libraries” are generated from environmental samples and represent the collective genomes of naturally occurring organisms archived in cloning vectors that can be propagated in suitable prokaryotic hosts. In this aspect, because the cloned DNA is initially extracted directly from environmental samples, the libraries are not limited to the small fraction of prokaryotes that can be grown in pure culture. In one aspect, a normalization of the environmental DNA present in these samples allows more equal representation of the DNA from all of the species present in the original sample; this can dramatically increase the efficiency of finding interesting genes from minor constituents of the sample which may be under-represented by several orders of magnitude compared to the dominant species.

In one aspect, gene libraries generated from one or more uncultivated microorganisms are screened for an activity of interest. Potential pathways encoding bioactive molecules of interest are first captured in prokaryotic cells in the form of gene expression libraries. In one aspect, polynucleotides encoding activities of interest are to isolated from such libraries and introduced into a host cell. The host cell is grown under conditions which promote recombination and/or reductive reassortment creating potentially active biomolecules with novel or enhanced activities.

In vivo biopanning may be performed utilizing a FACS-based and non-optical (e.g., magnetic) based machines. In one aspect, complex gene libraries are constructed with vectors which contain elements which stabilize transcribed RNA. For example, the inclusion of sequences which result in secondary structures such as hairpins which are designed to flank the transcribed regions of the RNA would serve to enhance their stability, thus increasing their half life within the cell. The probe molecules used in the biopanning process consist of oligonucleotides labeled with reporter molecules that only fluoresce upon binding of the probe to a target molecule. These probes are introduced into the recombinant cells from the library using one of several transformation methods. The probe molecules bind to the transcribed target mRNA resulting in DNA/RNA heteroduplex molecules. Binding of the probe to a target will yield a fluorescent signal which is detected and sorted by the FACS machine during the screening process.

In one aspect, subcloning is performed to further isolate sequences of interest. In subcloning, a portion of DNA is amplified, digested, generally by restriction enzymes, to cut out the desired sequence, the desired sequence is ligated into a recipient vector and is amplified. At each step in subcloning, the portion is examined for the activity of interest, in order to ensure that DNA that encodes the structural protein has not been excluded. The insert may be purified at any step of the subcloning, for example, by gel electrophoresis prior to ligation into a vector or where cells containing the recipient vector and cells not containing the recipient vector are placed on selective media containing, for example, an antibiotic, which will kill the cells not containing the recipient vector. Specific methods of subcloning cDNA inserts into vectors are well-known in the art (Sambrook et al., Molecular Cloning: A Laboratory Manual, 2nd Ed., Cold Spring Harbor Laboratory Press (1989)). In another aspect, the enzymes of the invention are subclones. Such subclones may differ from the parent clone by, for example, length, a mutation, a tag or a label.

The microorganisms from which the polynucleotide may be discovered, isolated or prepared include prokaryotic microorganisms, such as Eubacteria and Archaebacteria and lower eukaryotic microorganisms such as fungi, some algae and protozoa. Polynucleotides may be discovered, isolated or prepared from samples, e.g. environmental samples, in which case the nucleic acid may be recovered without culturing of an organism or recovered from one or more cultured organisms. In one aspect, such microorganisms may be extremophiles, such as hyperthermophiles, psychrophiles, psychrotrophs, halophiles, barophiles and acidophiles. Polynucleotides encoding enzymes isolated from extremophilic microorganisms can be used. Enzymes of this invention can function at temperatures above 100° C., e.g., as those found in is terrestrial hot springs and deep sea thermal vents, or at temperatures below 0° C., e.g., as those found in arctic waters, in a saturated salt environment, e.g., as those found in the Dead Sea, at pH values around 0, e.g., as those found in coal deposits and geothermal sulfur-rich springs, or at pH values greater than 11, e.g., as those found in sewage sludge. In one aspect, enzymes of the invention have high activity throughout a wide range of temperatures and pHs.

Polynucleotides selected and isolated as hereinabove described are introduced into a suitable host cell. A suitable host cell is any cell which is capable of promoting recombination and/or reductive reassortment. The selected polynucleotides are in one aspect already in a vector which includes appropriate control sequences. The host cell can be a higher eukaryotic cell, such as a mammalian cell, or a lower eukaryotic cell, such as a yeast cell, or in one aspect, the host cell can be a prokaryotic cell, such as a bacterial cell. Introduction of the construct into the host cell can be effected by calcium phosphate transfection, DEAE-Dextran mediated transfection, or electroporation.

Exemplary hosts include bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; and plant cells; see discussion, above. The selection of an appropriate host is deemed to be within the scope of those skilled in the art from the teachings herein.

Various mammalian cell culture systems can be employed to express recombinant protein; examples of mammalian expression systems include the COS-7 lines of monkey kidney fibroblasts, described in “SV40-transformed simian cells support the replication of early SV40 mutants” (Gluzman, 1981) and other cell lines capable of expressing a compatible vector, for example, the C127, 3T3, CHO, HeLa and BHK cell lines. Mammalian expression vectors can comprise an origin of replication, a suitable promoter and enhancer and also any necessary ribosome binding sites, polyadenylation site, splice donor and acceptor sites, transcriptional termination sequences and 5′ flanking nontranscribed sequences. DNA sequences derived from the SV40 splice and polyadenylation sites may be used to provide the required nontranscribed genetic elements.

In another aspect, nucleic acids, polypeptides and methods of the invention are used in biochemical pathways, or to generate novel polynucleotides encoding biochemical pathways from one or more operons or gene clusters or portions thereof. For example, bacteria and many eukaryotes have a coordinated mechanism for regulating genes whose products are involved in related processes. The genes are clustered, in structures referred to as “gene clusters,” on a single chromosome and are transcribed together under the control of a single regulatory sequence, including a single promoter which initiates transcription of the entire cluster. Thus, a gene cluster is a group of adjacent genes that are either identical or related, usually as to their function (an example of a biochemical pathway encoded by gene clusters are polyketides).

In one aspect, gene cluster DNA is isolated from different organisms and ligated into vectors, e.g., vectors containing expression regulatory sequences which can control and regulate the production of a detectable protein or protein-related array activity from the ligated gene clusters. Use of vectors which have an exceptionally large capacity for exogenous DNA introduction can be appropriate for use with such gene clusters and are described by way of example herein to include the f-factor (or fertility factor) of E. coli. This f-factor of E. coli is a plasmid which affects high-frequency transfer of itself during conjugation and is ideal to achieve and stably propagate large DNA fragments, such as gene clusters from mixed microbial samples. One aspect is to use cloning vectors, referred to as “fosmids” or bacterial artificial chromosome (BAC) vectors. These are derived from E. coli f-factor which is able to stably integrate large segments of genomic DNA. When integrated with DNA from a mixed uncultured environmental sample, this makes it possible to achieve large genomic fragments in the form of a stable “environmental DNA library.” Another type of vector for use in the present invention is a cosmid vector. Cosmid vectors were originally designed to clone and propagate large segments of genomic DNA. Cloning into cosmid vectors is described in detail in Sambrook et al., Molecular Cloning: A Laboratory Manual, 2nd Ed., Cold Spring Harbor Laboratory Press (1989). Once ligated into an appropriate vector, two or more vectors containing different polyketide synthase gene clusters can be introduced into a suitable host cell. Regions of partial sequence homology shared by the gene clusters will promote processes which result in sequence reorganization resulting in a hybrid gene cluster. The novel hybrid gene cluster can then be screened for enhanced activities not found in the original gene clusters.

Methods for screening for various enzyme activities are known to those of skill in the art and are discussed throughout the present specification, see, e.g., Examples 1, 2 and 3, below. Such methods may be employed when isolating the polypeptides and polynucleotides of the invention.

In one aspect, the invention provides methods for discovering and isolating cellulases, e.g., endoglucanase, cellobiohydrolase, beta-glucosidase, xylanase, mannanse, β-xylosidase and/or arabinofuranosidase, or compounds to modify the activity of these enzymes, using a whole cell approach (see discussion, below). clones encoding the lignocellulosic enzyme, e.g., glycosyl hydrolase, cellulase, endoglucanase, cellobiohydrolase, beta-glucosidase, xylanase, mannanse, β-xylosidase and/or arabinofuranosidase from genomic DNA library can be screened.

### Screening Methodologies and “On-line” Monitoring Devices

In practicing the methods of the invention, a variety of apparatus and methodologies can be used to in conjunction with the polypeptides and nucleic acids of the invention, e.g., to screen polypeptides for the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme activity, to screen compounds as potential modulators, e.g., activators or inhibitors, of a lignocellulosic enzyme activity, for antibodies that bind to a polypeptide of the invention, for nucleic acids that hybridize to a nucleic acid of the invention, to screen for cells expressing a polypeptide of the invention and the like. In addition to the array formats described in detail below for screening samples, alternative formats can also be used to practice the methods of the invention. Such formats include, for example, mass spectrometers, chromatographs, e.g., high-throughput HPLC and other forms of liquid chromatography, and smaller formats, such as 1536-well plates, 384-well plates and so on. High throughput screening apparatus can be adapted and used to practice the methods of the invention, see, e.g., U.S. Patent Application Nos. 20020001809; 20050272044.

Capillary Arrays

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. Capillary arrays, such as the GIGAMATRIX™, Verenium Corporation, San Diego, Calif.; and arrays described in, e.g., U.S. Patent Application No. 20020080350 A1; WO 0231203 A; WO 0244336 A, provide an alternative apparatus for holding and screening samples. In one aspect, the capillary array includes a plurality of capillaries formed into an array of adjacent capillaries, wherein each capillary comprises at least one wall defining a lumen for retaining a sample. The lumen may be cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. Additionally, the capillary array can include interstitial material disposed between adjacent capillaries in the array, thereby forming a solid planar device containing a plurality of through-holes.

A capillary array can be formed of any number of individual capillaries, for example, a range from 100 to 4,000,000 capillaries. Further, a capillary array having about 100,000 or more individual capillaries can be formed into the standard size and shape of a Microtiter® plate for fitment into standard laboratory equipment. The lumens are filled manually or automatically using either capillary action or microinjection using a thin needle. Samples of interest may subsequently be removed from individual capillaries for further analysis or characterization. For example, a thin, needle-like probe is positioned in fluid communication with a selected capillary to either add or withdraw material from the lumen.

In a single-pot screening assay, the assay components are mixed yielding a solution of interest, prior to insertion into the capillary array. The lumen is filled by capillary action when at least a portion of the array is immersed into a solution of interest. Chemical or biological reactions and/or activity in each capillary are monitored for detectable events. A detectable event is often referred to as a “hit”, which can usually be distinguished from “non-hit” producing capillaries by optical detection. Thus, capillary arrays allow for massively parallel detection of “hits”.

In a multi-pot screening assay, a polypeptide or nucleic acid, e.g., a ligand, can be introduced into a first component, which is introduced into at least a portion of a capillary of a capillary array. An air bubble can then be introduced into the capillary behind the first component. A second component can then be introduced into the capillary, wherein the second component is separated from the first component by the air bubble. The first and second components can then be mixed by applying hydrostatic pressure to both sides of the capillary array to collapse the bubble. The capillary array is to then monitored for a detectable event resulting from reaction or non-reaction of the two components.

In a binding screening assay, a sample of interest can be introduced as a first liquid labeled with a detectable particle into a capillary of a capillary array, wherein the lumen of the capillary is coated with a binding material for binding the detectable particle to the lumen. The first liquid may then be removed from the capillary tube, wherein the bound detectable particle is maintained within the capillary, and a second liquid may be introduced into the capillary tube. The capillary is then monitored for a detectable event resulting from reaction or non-reaction of the particle with the second liquid.

Arrays, or “Biochips”

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme gene. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins. The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface, as discussed in further detail, below.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

### Antibodies and Antibody-Based Screening Methods

The invention provides isolated, synthetic or recombinant antibodies that specifically bind to a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme of the invention. These antibodies can be used to isolate, identify or quantify the lignocellulosic enzyme of the invention or related polypeptides. These antibodies can be used to isolate other polypeptides within the scope the invention or other related the lignocellulosic enzymes. The antibodies can be designed to bind to an active site of a lignocellulosic enzyme. Thus, the invention provides methods of inhibiting the lignocellulosic enzyme using the antibodies of the invention (see discussion above regarding applications for anti-cellulase, e.g., anti-endoglucanase, anti-cellobiohydrolase and/or anti-beta-glucosidase enzyme compositions of the invention).

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The invention provides fragments of the enzymes of the invention (e.g., peptides) including immunogenic fragments (e.g., subsequences) of a polypeptide of the invention. The invention provides compositions comprising a polypeptide or peptide of the invention and adjuvants or carriers and the like.

The antibodies can be used in immunoprecipitation, staining, immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

The polypeptides of the invention or fragments comprising at least 5, 10, 15, 20, 25, 30, 35, 40, 50, 75, 100, or 150 consecutive amino acids thereof, may also be used to generate antibodies which bind specifically to the polypeptides or fragments. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention, or fragments comprising at least 5, 10, 15, 20, 25, 30, 35, 40, 50, 75, 100, or 150 consecutive amino acids thereof.

In immunoaffinity procedures, the antibody is attached to a solid support, such as a bead or other column matrix. The protein preparation is placed in contact with the antibody under conditions in which the antibody specifically binds to one of the polypeptides of the invention, or fragment thereof. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays and Western Blots.

Polyclonal antibodies generated against the polypeptides of the invention, or fragments comprising at least 5, 10, 15, 20, 25, 30, 35, 40, 50, 75, 100, or 150 consecutive amino acids thereof can be obtained by direct injection of the polypeptides into an animal or by administering the polypeptides to an animal, for example, a nonhuman. The antibody so obtained can bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique (Kohler and Milstein, Nature, 256:495-497, 1975), the trioma technique, the human B-cell hybridoma technique (Kozbor et al., Immunology Today 4:72, 1983) and the EBV-hybridoma technique (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to the polypeptides of the invention, or fragments comprising at least 5, 10, 15, 20, 25, 30, 35, 40, 50, 75, 100, or 150 consecutive amino acids thereof. Alternatively, transgenic mice may be used to express humanized antibodies to these polypeptides or fragments thereof.

Antibodies generated against the polypeptides of the invention, or fragments comprising at least 5, 10, 15, 20, 25, 30, 35, 40, 50, 75, 100, or 150 consecutive amino acids thereof may be used in screening for similar polypeptides from other organisms and samples. In such techniques, polypeptides from the organism are contacted with the antibody and those polypeptides which specifically bind the antibody are detected. Any of the procedures described above may be used to detect antibody binding. One such screening assay is described in “Methods for Measuring Cellulase Activities”, Methods in Enzymology, Vol 160, pp. 87-116.

### Kits

The invention provides kits comprising the compositions, e.g., nucleic acids, expression cassettes, vectors, cells, transgenic seeds or plants or plant parts, polypeptides (e.g., a cellulase enzyme) and/or antibodies of the invention. The kits also can contain instructional material teaching the methodologies and industrial, medical and dietary uses of the invention, as described herein.

### Whole Cell Engineering and Measuring Metabolic Parameters

The methods of the invention provide whole cell evolution, or whole cell engineering, of a cell to develop a new cell strain having a new phenotype, e.g., a new or modified the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme activity, by modifying the genetic composition of the cell. See U.S. patent application no. 20040033975.

The genetic composition can be modified by addition to the cell of a nucleic acid of the invention, e.g., a coding sequence for an enzyme of the invention. See, e.g., WO0229032; WO0196551.

To detect the new phenotype, at least one metabolic parameter of a modified cell is monitored in the cell in a “real time” or “on-line” time frame. In one aspect, a plurality of cells, such as a cell culture, is monitored in “real time” or “on-line.” In one aspect, a plurality of metabolic parameters is monitored in “real time” or “on-line.” Metabolic parameters can be monitored using the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the invention.

Metabolic flux analysis (MFA) is based on a known biochemistry framework. A linearly independent metabolic matrix is constructed based on the law of mass conservation and on the pseudo-steady state hypothesis (PSSH) on the intracellular metabolites. In practicing the methods of the invention, metabolic networks are established, including the:
identity of all pathway substrates, products and intermediary metabolites
identity of all the chemical reactions interconverting the pathway metabolites, the stoichiometry of the pathway reactions,
identity of all the enzymes catalyzing the reactions, the enzyme reaction kinetics,
the regulatory interactions between pathway components, e.g. allosteric interactions, enzyme-enzyme interactions etc,
intracellular compartmentalization of enzymes or any other supramolecular organization of the enzymes, and,
the presence of any concentration gradients of metabolites, enzymes or effector molecules or diffusion barriers to their movement.

Once the metabolic network for a given strain is built, mathematic presentation by matrix notion can be introduced to estimate the intracellular metabolic fluxes if the on-line metabolome data is available. Metabolic phenotype relies on the changes of the whole metabolic network within a cell. Metabolic phenotype relies on the change of pathway utilization with respect to environmental conditions, genetic regulation, developmental state and the genotype, etc. In one aspect of the methods of the invention, after the on-line MFA calculation, the dynamic behavior of the cells, their phenotype and other properties are analyzed by investigating the pathway utilization. For example, if the glucose supply is increased and the oxygen decreased during the yeast fermentation, the utilization of respiratory pathways will be reduced and/or stopped, and the utilization of the fermentative pathways will dominate. Control of physiological state of cell cultures will become possible after the pathway analysis. The methods of the invention can help determine how to manipulate the fermentation by determining how to change the substrate supply, temperature, use of inducers, etc. to control the physiological state of cells to move along desirable direction. In practicing the methods of the invention, the MFA results can also be compared with transcriptome and proteome data to design experiments and protocols for metabolic engineering or gene shuffling, etc.

In practicing the methods of the invention, any modified or new phenotype can be conferred and detected, including new or improved characteristics in the cell. Any aspect of metabolism or growth can be monitored.

Monitoring Expression of an mRNA Transcript

In one aspect of the invention, the engineered phenotype comprises increasing or decreasing the expression of an mRNA transcript (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme message) or generating new (e.g., the lignocellulosic enzyme transcripts in a cell. This increased or decreased expression can be traced by testing for the presence of a lignocellulosic enzyme of the invention or by the lignocellulosic enzyme activity assays. mRNA transcripts, or messages, also can be detected and quantified by any method known in the art, including, e.g., Northern blots, quantitative amplification reactions, hybridization to arrays, and the like. Quantitative amplification reactions include, e.g., quantitative PCR, including, e.g., quantitative reverse transcription polymerase chain reaction, or RT-PCR; quantitative real time RT-PCR, or “real-time kinetic RT-PCR” (see, e.g., Kreuzer (2001) Br. J. Haematol. 114:313-318; Xia (2001) Transplantation 72:907-914).

In one aspect of the invention, the engineered phenotype is generated by knocking out expression of a homologous gene. The gene's coding sequence or one or more transcriptional control elements can be knocked out, e.g., promoters or enhancers. Thus, the expression of a transcript can be completely ablated or only decreased.

In one aspect of the invention, the engineered phenotype comprises increasing the expression of a homologous gene. This can be effected by knocking out of a negative control element, including a transcriptional regulatory element acting in cis- or trans- , or, mutagenizing a positive control element. One or more, or, all the transcripts of a cell can be measured by hybridization of a sample comprising transcripts of the cell, or, nucleic acids representative of or complementary to transcripts of a cell, by hybridization to immobilized nucleic acids on an array.

Monitoring Expression of a Polypeptides, Peptides and Amino Acids

In one aspect of the invention, the engineered phenotype comprises increasing or decreasing the expression of a polypeptide (e.g., a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme) or generating new polypeptides in a cell. This increased or decreased expression can be traced by determining the amount of the lignocellulosic enzyme present or by the lignocellulosic enzyme activity assays. Polypeptides, peptides and amino acids also can be detected and quantified by any method known in the art, including, e.g., nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Furthermore, as discussed below in detail, one or more, or, all the polypeptides of a cell can be measured using a protein array.

### Industrial, Energy, Pharmaceutical and Other Applications

Polypeptides of the invention (e.g., having the lignocellulosic enzyme, e.g., glycosyl hydrolase, cellulase, endoglucanase, cellobiohydrolase, beta-glucosidase, xylanase, mannanse, β-xylosidase and/or arabinofuranosidase) can catalyze the breakdown of cellulose. The enzymes of the invention can be highly selective catalysts. The invention provides industrial processes using enzymes of the invention, e.g., in the pharmaceutical or nutrient (diet) supplement industry, the energy industry (e.g., to make “clean” biofuels), in the food and feed industries, e.g., in methods for making food and feed products and food and feed additives. In one aspect, the invention provides processes using enzymes of the invention in the medical industry, e.g., to make pharmaceuticals or dietary aids or supplements, or food supplements and additives. In addition, the invention provides methods for using the enzymes of the invention in biofuel production, including, e.g., a bioalcohol such as bioethanol, biomethanol, biobutanol or biopropanol, thus comprising a “clean” fuel production.

The enzymes of the invention can catalyze reactions with exquisite stereo-, regio- and chemo-selectivities. The lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the invention can be engineered to function in various solvents, operate at extreme pHs (for example, high pHs and low pHs) extreme temperatures (for example, high temperatures and low temperatures), extreme salinity levels (for example, high salinity and low salinity) and catalyze reactions with compounds that are structurally unrelated to their natural, physiological substrates.

Biomass Conversion and Production of Clean Bio Fuels

The invention provides enzymes (including mixtures, or “cocktails” of enzymes) and methods for the conversion of a biomass or any lignocellulosic material (e.g., any composition comprising cellulose, hemicellulose and lignin), to fermentable sugars, and/or monomeric sugars—and eventually to fuels (e.g., bioethanol, methanol, propanol, butanol) and the like), feeds, foods and chemicals or any other useful product. Thus, the compositions and methods of the invention provide effective and sustainable alternatives or adjuncts to use of petroleum-based products, e.g., as a mixture of a biofuel (e.g., an alcohol such as bioethanol, propanol, butanol, methanol and the like) and gasoline.

The invention provides organisms expressing enzymes and antibodies of the invention, e.g., as cell, cell culture, or transgenic plant or plant part (e.g., a seed or fruit) “production factories” for the synthesis of polypeptides of the invention (e.g., as means for the upscale, high yield manufacturing of polypeptides of the invention), or for participation of the enzyme or antibody of the invention in chemical cycles involving a natural biomass conversion, processing or other manipulation.

In one aspect, enzymes and methods for the conversion are used in enzyme ensembles (“mixtures” or “cocktails”) for the efficient hydrolysis (e.g., depolymerization) of lignocellulosic, cellulosic and/or hemicellulosic polymers to metabolizeable carbon moieties, including sugars and alcohols. Exemplary enzyme cocktails are described herein; however, the invention encompasses compositions comprising mixtures of enzymes comprising at least one (any combination of) enzyme(s) of the invention; and in alternative embodiments, a mixture (“ensembles” or “cocktails”) of the invention can also comprise any other enzyme, e.g., a glucose oxidase, a phosphorylase, and amidase, etc., and the like. As discussed above, the invention provides methods for discovering and implementing the most effective of enzymes to enable these important new “biomass conversion”, “biomass processing” and alternative energy, or biofuel production, industrial processes.

In one aspect, polypeptides of the invention having lignocellulosic activity, e.g., glucosyl hydrolase, cellulase, endoglucanase, cellobiohydrolase and/or β-glucosidase (beta-glucosidase) activity, are used in processes for converting lignocellulosic biomass to monomeric sugars, which are eventually converted to a bioalcohol, e.g., ethanol, methanol, etc. Thus, the invention provides processes for making biofuels comprising, e.g., a bioalcohol such as bioethanol, biomethanol, biobutanol or biopropanol, from compositions comprising lignocellulosic biomass. The lignocellulose biomass material is can be obtained from agricultural crops, as a byproduct of food or feed production, or as lignocellulosic waste products, such as plant residues (e.g., sugarcane bagasse or corn fiber, such as corn seed fiber) and waste paper. Examples of suitable plant residues for treatment with polypeptides of the invention include sugarcane (e.g., bagasse, cane tops), grains, seeds, stems, leaves, hulls, husks, corn or corn cobs, corn stover, corn fiber, hay or straw (e.g., a rice straw or a wheat straw, or any the dry stalk of any cereal plant), grasses (e.g., Indian grass, such as Sorghastrum nutans; or, switch grass, e.g., Panicum species, such as Panicum virgatum), sugar beet pulp, citrus pulp, citrus peels, and the like, as well as wood, wood thinnings, wood waste, wood chips, wood pulp, pulp waste, wood waste, wood shavings, sawdust, construction and/or demolition wastes and debris (e.g. wood, wood shavings and sawdust). Examples of paper or wood waste suitable for treatment with polypeptides of the invention include discarded or used photocopy paper, computer printer paper, notebook paper, notepad paper, typewriter paper, and the like, as well as newspapers, magazines, cardboard, and paper-based packaging materials and recycled paper materials. In addition, urban wastes, e.g. the paper fraction of municipal solid waste, municipal wood waste, and municipal green waste, along with other materials containing sugar, starch, and/or cellulose can be used

The enzymes of the invention used to treat or process the lignocellulose biomass material (e.g., from agricultural crops, food or feed production byproduct, lignocellulosic waste products, plant residues, sugarcane bagasse, corn or corn fiber, waste wood or paper, etc.), in addition to being directly added to the material, alternatively can be made by a microorganism (e.g., a virus, plant, yeast, etc.) living on or within the biomass material, or by the biomass material itself, e.g., as a transgenic plant or seed and the like. In one aspect, microorganisms that produce the enzyme (e.g., by spraying, infecting, etc.) are added to the biomass material to be processed—this can be the sole source of the enzyme, or can supplement enzyme that is added in another form (e.g., as either a purified enzyme, or in crude lysate of a culture, such as a bacterial, yeast or insect cell culture, or any other formulation), or to supplement the presence of the enzyme as a heterologous recombinant protein in a transgenic plant. Alternatively, io the plant can be engineered to express the enzyme recombinantly by transient infection, transformation or transduction with naked DNA, plasmid, virus and the like. Alternatively, the enzymes are produced in plants or plant seeds, like corn, and then the enzyme can be isolated from the plant or the plant can be used directly in the process. In alternative embodiments, the enzymes of the invention can be added to the treatment process in batches, by fed-batch processes, added continually and/or be recycled during the process.

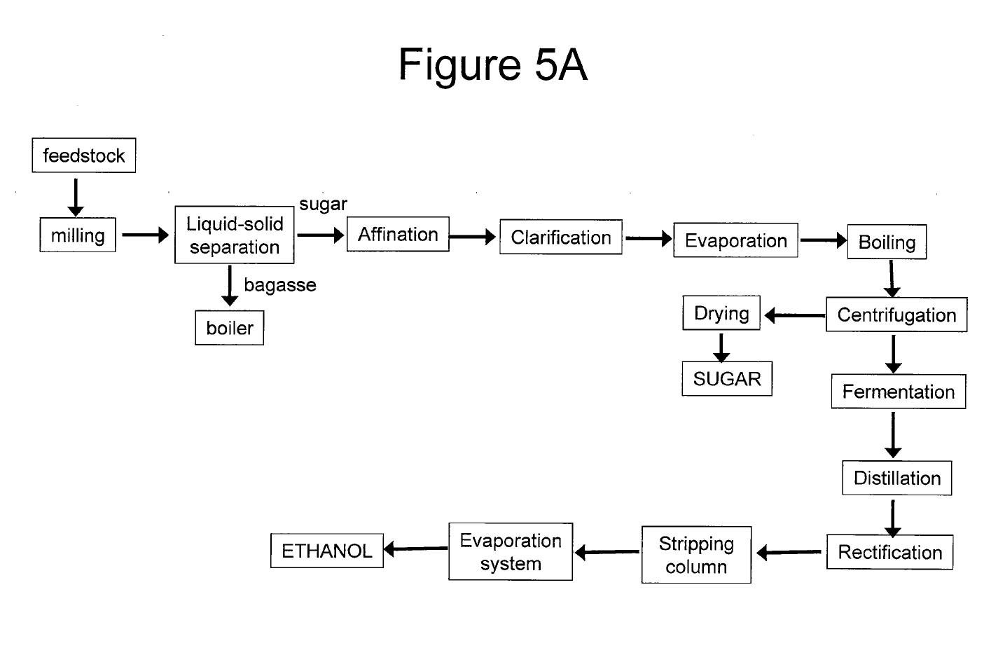

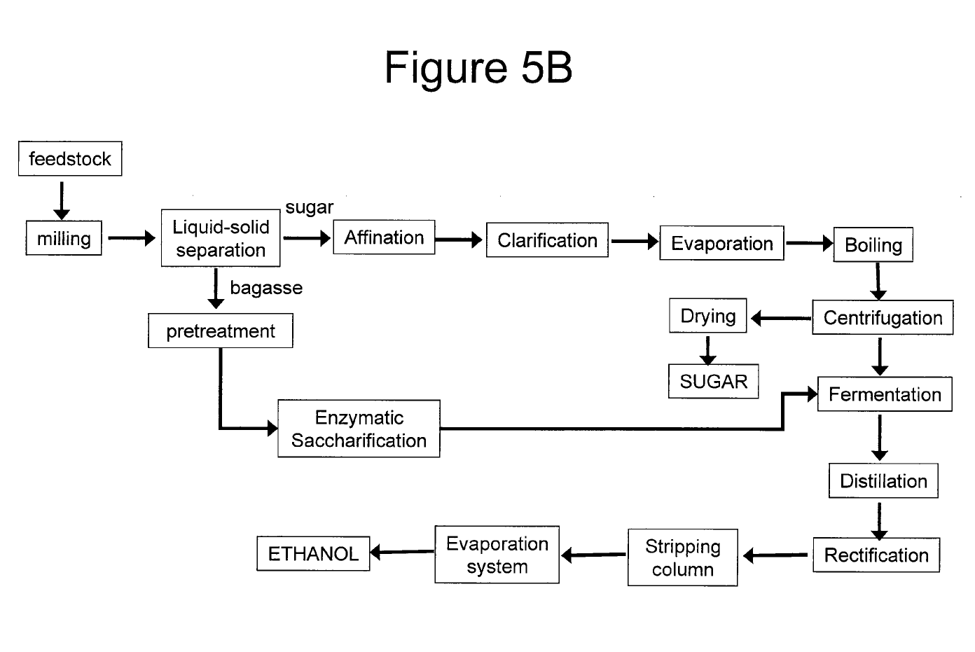

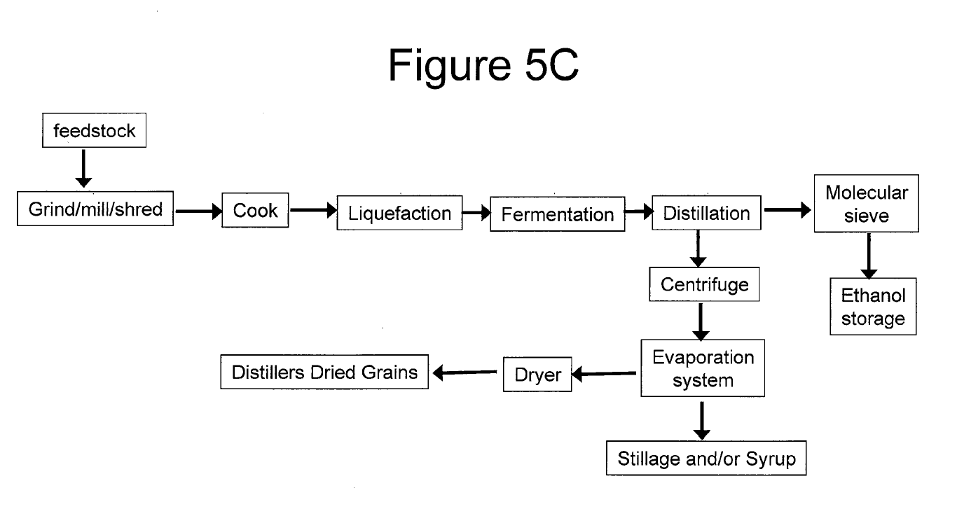

Enzymes and methods of the invention can be used in conjunction with any sugar production process, e.g., in a typical cane sugar production plant, where sugarcane processing is focused on the production of cane sugar (sucrose) from sugarcane; e.g., as illustrated in FIGS. 5A and 5B (both exemplary feedstock to sugar to bioalcohol, e.g., ethanol, methanol, etc., processes of the invention) and 5C (an exemplary dry milling process of the invention). One or more polypeptides (e.g., enzymes) of the invention can be added in one, any, some, or all of the steps illustrated in FIGS. 5A, 5B and/or 5C. Other products of these exemplary processes of the invention can include; ethanol, bagasse, and molasses. In one aspect, bagasse, the residual fibrous component of the sugarcane is used as a fuel source for the boilers in the generation of process steam. In alternative aspects, molasses is produced in two forms: inedible form (edible for animals; blackstrap) or as (human) edible syrup. Blackstrap molasses is used primarily as an animal feed additive, but it is also used to produce ethanol. Edible molasses syrups can be blended with maple syrup, invert sugars, or corn syrup.

In one exemplary process, the cane is received at the mill and prepared for the extraction of the juice. The milling process can occur in two steps: breaking the hard structure of the cane and grinding the cane. Imbibition is the process in which water is applied to the crushed cane to enhance the extraction of the juice. The leftover material after the crushing step is called bagasse, which is burnt in the boilers to produce steam and electricity. The extracted juice is strained to remove large particles and then clarified. In raw sugar production, the clarification is done almost exclusively with heat and lime, and small quantities of soluble phosphate also may be added. The lime is added to neutralize the organic acids, and the temperature is raised to approximately 95° C. A heavy precipitate is formed, which is separated from the juice in the clarifier. Clarified juice is transferred to the evaporators without further treatment. Evaporation is performed in two stages: initially in an evaporator to concentrate the juice and then in vacuum pans to crystallize the sugar. The evaporator station typically produces syrup with about 65% solids and 35% water. Following evaporation, the syrup is clarified by adding lime, phosphoric acid and a polymer flocculent, aerated, and filtered in the clarifier. From the clarifier, the syrup goes to the vacuum pans for crystallization. In the pans, the syrup is evaporated and the crystallization process is initiated. When the volume of the mixture of liquor and crystals, known as massecuite reaches capacity the contents are discharged to the crystallizer. From the crystallizer, the massecuite A is transferred to high speed centrifugal machine, in which the liquor (A molasses) is separated from the crystals. A molasses is returned to a vacuum pan and reboiled to yield B massecuite that yields a second batch of crystals and B molasses after centrifugation. B molasses is much lower purity than A molasses and it undergoes reboiling to form a lower grade massecuite C, which goes to a crystallizer and then to a centrifugal. The final molasses from the third stage (blackstrap molasses) is a heavy, viscous material used primarily to produce ethanol and as an additive in cattle feed. The cane sugar from the combined A and B massecuite is cooled and transported to sugar refinery.

In one aspect, the enzymes and methods of the invention can be used in conjunction with more “traditional” means of making a bioalcohol, e.g., ethanol, methanol, etc., from biomass, e.g., as methods comprising hydrolyzing lignocellulosic materials by subjecting dried lignocellulosic material in a reactor to a catalyst comprised of a dilute solution of a strong acid and a metal salt; this can lower the activation energy, or the temperature, of cellulose hydrolysis to obtain higher sugar yields; see, e.g., U.S. Pat. Nos. 6,660,506; 6,423,145.

Another exemplary method that incorporated use of enzymes of the invention comprises hydrolyzing lignocellulosic material containing hemicellulose, cellulose and lignin by subjecting the material to a first stage hydrolysis step in an aqueous medium at a temperature and a pressure chosen to effect primarily depolymerization of hemicellulose without major depolymerization of cellulose to glucose. This step results in a slurry in which the liquid aqueous phase contains dissolved monosaccharides resulting from depolymerization of hemicellulose and a solid phase containing cellulose and lignin. A second stage hydrolysis step can comprise conditions such that at least a major portion of the cellulose is depolymerized, such step resulting in a liquid aqueous phase containing dissolved/ soluble depolymerization products of cellulose. See, e.g., U.S. Pat. No. 5,536,325. Enzymes of the invention can be added at any stage of this exemplary process.

Another exemplary method that incorporated use of enzymes of the invention comprises processing a lignocellulose-containing biomass material by one or more stages of dilute acid hydrolysis with about 0.4% to 2% strong acid; and treating an unreacted solid lignocellulosic component of the acid hydrolyzed biomass material by alkaline delignification to produce precursors for biodegradable thermoplastics and derivatives. See, e.g., U.S. Pat. No. 6,409,841. Enzymes of the invention can be added at any stage of this exemplary process.

Another exemplary method that incorporated use of enzymes of the invention comprises prehydrolyzing lignocellulosic material in a prehydrolysis reactor; adding an acidic liquid to the solid lignocellulosic material to make a mixture; heating the mixture to reaction temperature; maintaining reaction temperature for time sufficient to fractionate the lignocellulosic material into a solubilized portion containing at least about 20% of the lignin from the lignocellulosic material and a solid fraction containing cellulose; removing a solubilized portion from the solid fraction while at or near reaction temperature wherein the cellulose in the solid fraction is rendered more amenable to enzymatic digestion; and recovering a solubilized portion. See, e.g., U.S. Pat. No. 5,705,369. Enzymes of the invention can be added at any stage of this exemplary process.

The invention provides methods for making motor fuel compositions (e.g., for spark ignition motors) based on liquid hydrocarbons blended with a fuel grade alcohol made by using an enzyme or a method of the invention. In one aspect, the fuels made by use of an enzyme of the invention comprise, e.g., coal gas liquid- or natural gas liquid-ethanol blends. In one aspect, a co-solvent is biomass-derived 2-methyltetrahydrofuran (MTHF). See, e.g., U.S. Pat. No. 6,712,866.

Methods of the invention for the enzymatic degradation of lignocellulose, e.g., for production of sugars and/or ethanol from lignocellulosic material, can also comprise use of ultrasonic treatment of the biomass material; see, e.g., U.S. Pat. No. 6,333,181.

Another exemplary process for making a biofuel comprising a bioalcohol, e.g., ethanol, methanol, etc., using enzymes of the invention comprises pretreating a starting material comprising a lignocellulosic feedstock comprising at least hemicellulose and cellulose. In one aspect, the starting material comprises potatoes, soybean (rapeseed), barley, rye, corn, oats, wheat, beets or sugar cane or a component or waste or food or feed production byproduct. The starting material (“feedstock”) is reacted at conditions which disrupt the plant's fiber structure to effect at least a partial hydrolysis of the hemicellulose and cellulose. Disruptive conditions can comprise, e.g., subjecting the starting material to an average temperature of 180° C. to 270° C. at pH 0.5 to 2.5 for a period of about 5 seconds to 60 minutes; or, temperature of 220° C. to 270° C., at pH 0.5 to 2.5 for a period of 5 seconds to 120 seconds, or equivalent. This generates a feedstock with increased accessibility to being digested by an enzyme, e.g., a cellulase enzyme of the invention. U.S. Pat. No. 6,090,595.

Exemplary conditions for cellulase hydrolysis of lignocellulosic material include reactions at temperatures between about 30° C. and 48° C., and/or a pH between about 4.0 and 6.0. Other exemplary conditions include a temperature between about 30° C. and 60° C. and a pH between about 4.0 and 8.0.

Biofuels and Biologically Produced Alcohols

The invention provides biofuels and synthetic fuels, including liquids and gases (e.g., syngas) and biologically produced alcohols, and methods for making them, using the compositions (e.g., enzyme and nucleic acids, and transgenic plants, animal, seeds and microorganisms) and methods of the invention. The invention provides biofuels and biologically produced alcohols comprising enzymes, nucleic acids, transgenic plants, animals (e.g., microorganisms, such as bacteria or yeast) and/or seeds of the invention. In one aspect, these biofuels and biologically produced alcohols are produced from a biomass.

The invention provides biologically produced alcohols, such as ethanol, methanol, propanol and butanol produced by methods of the invention, which include the action of microbes and enzymes of the invention through fermentation (hydrolysis) to result in an alcohol fuel.

Biofuels as a Liquid or a Gas Gasoline

The invention provides biofuels and synthetic fuels in the form of a gas, or gasoline, e.g., a syngas. In one aspect, methods of the invention comprising use of enzymes of the invention for chemical cycles for natural biomass conversion, e.g., for the hydrolysis of a biomass to make a biofuel, e.g., a bioethanol, biopropanol, bio-butanol or a biomethanol, or a synthetic fuel, in the form of a liquid or as a gas, such as a “syngas”.

For example, invention provides methods for making biofuel gases and synthetic gas fuels (“syngas”) comprising a bioethanol, biopropanol, bio-butanol and/or a biomethanol made using a polypeptide of the invention, or made using a method of the invention; and in one aspect this biofuel gas of the invention is mixed with a natural gas (can also be produced from biomass), e.g., a hydrogen or a hydrocarbon-based gas fuel.

In one aspect, the invention provides methods for processing biomass to a synthetic fuel, e.g., a syngas, such as a syngas produced from a biomass by gasification. In one aspect, the invention provides methods for making an ethanol, propanol, butanol and/or methanol gas from a sugar cane, e.g., a bagasse. In one aspect, this fuel, or gas, is used as motor fuel, e.g., an automotive, truck, airplane, boat, small engine, etc. fuel. In one aspect, the invention provides methods for making an ethanol, propanol, butanol and/or methanol from a plant, e.g., corn, or a plant product, e.g., hay or straw (e.g., a rice straw or a wheat straw, or any the dry stalk of any cereal plant), or an agricultural waste product. Cellulosic ethanol, propanol, butanol and/or methanol can be manufactured from a plant, e.g., corn, or plant product, e.g., hay or straw, or an agricultural waste product (e.g., as processed by Iogen Corporation of Ontario, Canada).

In one aspect, the ethanol, propanol, butanol and/or methanol made using a method of composition of the invention can be used as a fuel (e.g., a gasoline) additive (e.g., an oxygenator) or in a direct use as a fuel. For example, a ethanol, propanol, butanol and/or methanol, including a fuel, made by a method of the invention can be mixed with ethyl tertiary butyl ether (ETBE), or an ETBE mixture such as ETBE containing 47% ethanol as a biofuel, or with MTBE (methyl tertiary-butyl ether). In another aspect, a ethanol, propanol, butanol and/or methanol, including a fuel, made by a method of the invention can be mixed with:

| IUPAC name | common name |
|---|---|
| but-1-ene | α-butylene |
| cis-but-2-ene | cis-β-butylene |
| trans-but-2-ene | trans-β-butylene |
| 2-methylpropene | Isobutylene |

A butanol and/or ethanol made by a method of the invention (e.g., using an enzyme of the invention) can be further processed using “A.B.E.” (Acetone, Butanol, Ethanol) fermentation; in one aspect, butanol being the only liquid product. In one aspect, this butanol and/or ethanol is burned “straight” in existing gasoline engines (without modification to the engine or car), produces more energy and is less corrosive and less water soluble than ethanol, and can be distributed via existing infrastructures.

The invention also provides mixed alcohols wherein one, several or all of the alcohols are made by processes comprising at least one method of the invention (e.g., using an enzyme of the invention), e.g., comprising a mixture of ethanol, propanol, butanol, pentanol, hexanol, and heptanol, such as ECALENE™ (Power Energy Fuels, Inc., Lakewood, Colo.), e.g.:

Exemplary Fuel of the Invention

| Component | Weight % |
|---|---|
| Methanol | 0% |
| Ethanol | 75% |
| Propanol | 9% |
| Butanol | 7% |
| Pentanol | 5% |
| Hexanol & Higher | 4% |

In one aspect, one, several or all of these alcohols are made by a process of the invention using an enzyme of the invention, and the process can further comprise a biomass-to-liquid technology, e.g., a gasification process to produce syngas followed by catalytic synthesis, or by a bioconversion of biomass to a mixed alcohol fuel.

The invention also provides processes comprising use of an enzyme of the invention incorporating (or, incorporated into) “gas to liquid”, or GTL; or “coal to liquid”, or CTL; or “biomass to liquid” or BTL; or “oilsands to liquid”, or OTL, processes; and in one aspect these processes of the invention are used to make synthetic fuels. In one aspect, one of these processes of the invention comprises making a biofuel (e.g., a synfuel) out of a biomass using, e.g., the so-called “Fischer Tropsch” process (a catalyzed chemical reaction in which carbon monoxide and hydrogen are converted into liquid hydrocarbons of various forms; typical catalysts used are based on iron and cobalt; the principal purpose of this process is to produce a synthetic petroleum substitute for use as synthetic lubrication oil or as synthetic fuel). In one aspect, this synthetic biofuel of the invention can contain oxygen and can be used as additive in high quality diesel and petrol.

Enzymatic Processes for Sugarcane Bagasse

The invention provides polypeptides that can enzymatically process (hydrolyze) sugarcane (Saccharum), sugarcane parts (e.g., cane tops) and/or sugarcane bagasse, i.e., for sugarcane degradation, or for biomass processing, and polynucleotides encoding these enzymes, and making and using these polynucleotides and polypeptides. The invention provides polypeptides and methods for processing lignocellulosic residues, including sugarcane bagasse, or any waste product of the sugar milling or related industries, into a lignocellulosic hydrolysis product, which itself can be a biofuel or which can be further processed to become a biofuel, including liquid or gas fuels. Because the invention provides enzymes and methods for sugar cane processing, it also provides methods for making (methods for the production of) edible sugar, garapa, rapadura (papelón), falernum, molasses, rum, cachaça, in addition to alcohols (for any purpose) and/or biofuels, e.g., bioethanol. Thus, the invention also provides edible sugar, garapa, rapadura (papelón), falernum, molasses, rum, cachaça, alcohols, biofuels, e.g., bioethanol and the like, and their intermediate, comprising a polypeptide of the invention.

In some aspects, are several advantages to using sugarcane, e.g., bagasse, as a substrate for bioconversion:
1. It has high carbohydrate content (cellulose, 40-50%, and hemicellulose, 20-30%);
2. It is collected at the site of processing;

3. It is a cheap substrate, and there is a constant, although seasonal supply generated within the sugarcane industry.

The invention provides polypeptides and methods for hydrolyzing cellulose and hemicellulose polysaccharides in sugarcane, e.g., bagasse, which are associated with lignin, which can act as a barrier shielding the polysaccharides from attack by microorganisms and their associated enzyme systems. Because of the structural characteristics of lignocellulose, such as its lignin barrier and cellulose crystallinity, in one aspect a pretreatment process is used to enhance the access of enzyme(s) of this invention to the polysaccharide components in a biomass (a bagasse) to increase the conversion yields into the building block monosaccharides, such as hexose and pentose sugars. In one exemplary system of this invention using enzyme(s) of this invention, sugars produced are efficiently fermented to ethanol, and burning unhydrolyzed carbohydrate plus lignin provides enough steam to fuel the sugar mills.

In alternative aspects, the processes of the invention use various pretreatments, which can be grouped into three categories: physical, chemical, and multiple (physical+chemical). Any chemicals can be used as a pretreatment agent, e.g., acids, alkalis, gases, cellulose solvents, alcohols, oxidizing agents and reducing agents. Among these chemicals, alkali is the most popular pretreatment agent because it is relatively inexpensive and results in less cellulose degradation. The common alkalis sodium hydroxide and lime also can be used as pretreatment agents. Although sodium hydroxide increases biomass digestibility significantly, it is difficult to recycle, is relatively expensive, and is dangerous to handle. In contrast, lime has many advantages: it is safe and very inexpensive, and can be recovered by carbonating wash water with carbon dioxide.

In one aspect, the invention provides a multi-enzyme system (including at least one enzyme of this invention) that can hydrolyze polysaccharides in a sugarcane, e.g., bagasse, component of sugarcane processed in sugar mills. In one aspect, the sugarcane, e.g., bagasse, is processed by an enzyme of the invention made by an organism (e.g., transgenic animal, plants, transformed microorganism) and/or byproduct (e.g., harvested plant, fruit, seed) expressing an enzyme of the invention. In one aspect, the enzyme is a recombinant enzyme made by the plant or biomass which is to be processed to a fuel, e.g., the invention provides a transgenic sugarcane bagasse comprising an enzyme of the invention. In one aspect, these compositions and products used in methods of the invention comprising chemical cycles for natural biomass conversion, e.g., for the hydrolysis of a biomass to make a biofuel, e.g., bioethanol, biopropanol, bio-butanol, biomethanol, a synthetic fuel in the form of a liquid or a gas, such as a “syngas”.

In one aspect, the invention provides a biofuel, e.g., a biogas, produced by the process of anaerobic digestion of organic material by anaerobes, wherein the process comprises use of an enzyme of the invention or a method of the invention. This biofuel, e.g., a biogas, can be produced either from biodegradable waste materials or by the use of energy crops fed into anaerobic digesters to supplement gas yields. The solid output, digestate, can also be used as a biofuel.

In one aspect, the invention provides a biofuel, e.g., a biogas, comprising a methane, wherein the process comprises use of an enzyme of the invention or a method of the invention. This biofuel, e.g., a biogas, can be recovered in industrial anaerobic digesters and mechanical biological treatment systems. Landfill gas can be further processed using an enzyme of this invention or a process of this invention; before processing landfill gas can be a less clean form of biogas produced in landfills through naturally occurring anaerobic digestion. Paradoxically if landfill gas is allowed to escape into the atmosphere it is a potent greenhouse gas.

The invention provides methods for making biologically produced oils and gases from various wastes, wherein the process comprises use of an enzyme of the invention or a method of the invention. In one aspect, these methods comprise thermal depolymerization of waste to extract methane and other oils similar to petroleum; or, e.g., a bioreactor system that utilizes nontoxic photosynthetic algae to take in smokestacks flue gases and produce biofuels such as biodiesel, biogas and a dry fuel comparable to coal, e.g., as designed by GreenFuel Technologies Corporation, of Cambridge, Mass.

The invention provides methods for making biologically produced oils, including crude oils, and gases that can be used in diesel engines, wherein the process comprises use of an enzyme of the invention or a method of the invention. In one aspect, these methods can refine petroleum, e.g., crude oils, into kerosene, petroleum, diesel and other fractions.

The invention provides methods (using an enzyme of the invention or a method of the invention) for making biologically produced oils from:
Straight vegetable oil (SVO).
Waste vegetable oil (WVO)—waste cooking oils and greases produced in quantity mostly by commercial kitchens.
Biodiesel obtained from transesterification of animal fats and vegetable oil, directly usable in petroleum diesel engines.
Biologically derived crude oil, together with biogas and carbon solids via the thermal depolymerization of complex organic materials including non oil based materials; for example, waste products such as old tires, offal, wood and plastic.
Pyrolysis oil; which may be produced out of biomass, wood waste etc. using heat only in the flash pyrolysis process (the oil may have to be treated before using in conventional fuel systems or internal combustion engines).
Wood, charcoal, and dried dung.

Animal Feeds and Food or Feed Additives

In addition to providing dietary aids or supplements, or food supplements and additives for human use, the invention also provides compositions and methods for treating animal feeds and foods and food or feed additives using a polypeptide of the invention, e.g., a protein having a lignocellulosic activity, e.g., a glycosyl hydrolase, cellulase, endoglucanase, cellobiohydrolase, beta-glucosidase, xylanase, mannanse, β-xylosidase and/or arabinofuranosidase enzymes of the invention, and/or the antibodies of the invention. The invention provides animal feeds, foods, and additives comprising the lignocellulosic enzymes of the invention and/or antibodies of the invention. The animal can be any farm animal or any animal.

The animal feed additive of the invention may be a granulated enzyme product that may readily be mixed with feed components. Alternatively, feed additives of the invention can form a component of a pre-mix. The granulated enzyme product of the invention may be coated or uncoated. The particle size of the enzyme granulates can be compatible with that of feed and pre-mix components. This provides a safe and convenient mean of incorporating enzymes into feeds. Alternatively, the animal feed additive of the invention may be a stabilized liquid composition. This may be an aqueous or oil-based slurry. See, e.g., U.S. Pat. No. 6,245,546.

The lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the present invention, in the modification of animal feed or a food, can process the food or feed either in vitro (by modifying components of the feed or food) or in vivo. Polypeptides of the invention can be added to animal feed or food compositions.

In one aspect, an enzyme of the invention is added in combination with another enzyme, e.g., beta-galactosidases, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, cellobiohydrolases, and/or glucose oxidases.

These enzyme digestion products are more digestible by the animal. Thus, the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes of the invention can contribute to the available energy of the feed or food, or to the digestibility of the food or feed by breaking down cellulose.

In another aspect, the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme of the invention can be supplied by expressing the enzymes directly in transgenic feed crops (as, e.g., transgenic plants, seeds and the like), to such as grains, cereals, corn, soy bean, rape seed, lupin and the like. As discussed above, the invention provides transgenic plants, plant parts and plant cells comprising a nucleic acid sequence encoding a polypeptide of the invention. In one aspect, the nucleic acid is expressed such that the lignocellulosic enzyme of the invention is produced in recoverable quantities. The lignocellulosic enzyme can be recovered from any plant or plant part. Alternatively, the plant or plant part containing the recombinant polypeptide can be used as such for improving the quality of a food or feed, e.g., improving nutritional value, palatability, etc.

In one aspect, the enzyme delivery matrix of the invention is in the form of discrete plural particles, pellets or granules. By “granules” is meant particles that are compressed or compacted, such as by a pelletizing, extrusion, or similar compacting to remove water from the matrix. Such compression or compacting of the particles also promotes intraparticle cohesion of the particles. For example, the granules can be prepared by pelletizing the grain-based substrate in a pellet mill. The pellets prepared thereby are ground or crumbled to a granule size suitable for use as an adjuvant in animal feed. Since the matrix is itself approved for use in animal feed, it can be used as a diluent for delivery of enzymes in animal feed.

In one aspect, the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzyme contained in the invention enzyme delivery matrix and methods is a thermostable the lignocellulosic enzyme, as described herein, so as to resist inactivation of the lignocellulosic enzyme during manufacture where elevated temperatures and/or steam may be employed to prepare the palletized enzyme delivery matrix. During digestion of feed containing the invention enzyme delivery matrix, aqueous digestive fluids will cause release of the active enzyme. Other types of thermostable enzymes and nutritional supplements that are thermostable can also be incorporated in the delivery matrix for release under any type of aqueous conditions.

In one aspect, a coating is applied to the enzyme matrix particles for many different purposes, such as to add a flavor or nutrition supplement to animal feed, to delay release of animal feed supplements and enzymes in gastric conditions, and the like. In one aspect, the coating is applied to achieve a functional goal, for example, whenever it is desirable to slow release of the enzyme from the matrix particles or to control the conditions under which the enzyme will be released. The composition of the coating material can be such that it is selectively broken down by an agent to which it is to susceptible (such as heat, acid or base, enzymes or other chemicals). Alternatively, two or more coatings susceptible to different such breakdown agents may be consecutively applied to the matrix particles.

The invention is also directed towards a process for preparing an enzyme-releasing matrix. In accordance with the invention, the process comprises providing discrete plural particles of a grain-based substrate in a particle size suitable for use as an enzyme-releasing matrix, wherein the particles comprise a lignocellulosic enzyme, e.g., a glycosyl hydrolase, cellulase, endoglucanase, cellobiohydrolase, beta-glucosidase, xylanase, mannanse, β-xylosidase and/or arabinofuranosidase enzyme encoded by an amino acid sequence of the invention. In one aspect, the process includes compacting or compressing the particles of enzyme-releasing matrix into granules, which most in one aspect is accomplished by pelletizing. The mold inhibitor and cohesiveness agent, when used, can be added at any suitable time, and in one aspect are mixed with the grain-based substrate in the desired proportions prior to pelletizing of the grain-based substrate. Moisture content in the pellet mill feed in one aspect is in the ranges set forth above with respect to the moisture content in the finished product, and in one aspect is about 14-15%. In one aspect, moisture is added to the feedstock in the form of an aqueous preparation of the enzyme to bring the feedstock to this moisture content. The temperature in the pellet mill in one aspect is brought to about 82° C. with steam. The pellet mill may be operated under any conditions that impart sufficient work to the feedstock to provide pellets. The pelleting process itself is a cost-effective process for removing water from the enzyme-containing composition.

The compositions and methods of the invention can be practiced in conjunction with administration of prebiotics, which are high molecular weight sugars, e.g., fructo-oligosaccharides (FOS); galacto-oligosaccharides (GOS), GRAS (Generally Recognized As Safe) material. These prebiotics can be metabolized by some probiotic lactic acid bacteria (LAB). They are non-digestible by the majority of intestinal microbes.

Treating Foods and Food Processing

The invention provides foods and feeds comprising enzymes of the invention, and methods for using enzymes of the invention in processing foods and feeds. Cellulases, e.g., endoglucanase, cellobiohydrolase, beta-glucosidase, xylanase, mannanse, β-xylosidase and/or arabinofuranosidase enzymes of the invention have numerous applications in food processing industry. The invention provides methods for hydrolyzing cellulose-comprising compositions, including, e.g., a plant cell, a bacterial cell, a yeast cell, an insect cell, or an animal cell, or any plant or plant part, or any food or feed, a waste product and the like.

For example, the invention provides feeds or foods comprising a lignocellulosic enzyme of the invention, e.g., in a feed, a liquid, e.g., a beverage (such as a fruit juice or a beer), a bread or a dough or a bread product, or a drink (e.g., a beer) or a beverage precursor (e.g., a wort).

The food treatment processes of the invention can also include the use of any combination of other enzymes such as tryptophanases or tyrosine decarboxylases, laccases, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, cellobiohydrolases, and/or glucose oxidases.

In one aspect, the invention provides enzymes and processes for hydrolyzing liquid (liquefied) and granular starch. Such starch can be derived from any source, e.g., beet, cane sugar, potato, corn, wheat, milo, sorghum, rye or bulgher. The invention applies to any plant starch source, e.g., a grain starch source, which is useful in liquefaction (for example, to make biofuels comprising, e.g., a bioalcohol such as bioethanol, biomethanol, biobutanol or biopropanol), including any other grain or vegetable source known to produce starch suitable for liquefaction. The methods of the invention comprise liquefying starch (e.g., making biofuels comprising, e.g., a bioalcohol such as bioethanol, biomethanol, biobutanol or biopropanol) from any natural material, such as rice, germinated rice, corn, barley, milo, wheat, legumes, potato, beet, cane sugar and sweet potato. The liquefying process can substantially hydrolyze the starch to produce a syrup. The temperature range of the liquefaction can be any liquefaction temperature which is known to be effective in liquefying starch. For example, the temperature of the starch can be between about 80° C. to about 115° C., between about 100° C. to about 110° C., and from about 105° C. to about 108° C. The bioalcohols made using the enzymes and processes of the invention can be used as fuels or in fuels (e.g., auto fuels), e.g., as discussed below, in addition to their use in (or for making) foods and feeds, including alcoholic beverages.

Waste Treatment

The invention provides enzymes for use in waste treatment. Cellulases, e.g., endoglucanase, cellobiohydrolase, beta-glucosidase, xylanase, mannanse, β-xylosidase and/or arabinofuranosidase enzymes of the invention can be used in a variety of waste treatment or related industrial applications, e.g., in waste treatment related to biomass conversion to generate fuels. For example, in one aspect, the invention provides a solid and/or liquid waste digestion process using the lignocellulosic enzyme of the invention. The methods can comprise reducing the mass and volume of substantially untreated solid waste. Solid waste can be treated with an enzymatic digestive process in the presence of an enzymatic solution (including the lignocellulosic enzymes of the invention) at a controlled temperature. This results in a reaction without appreciable bacterial fermentation from added microorganisms. The solid waste is converted into a liquefied waste and any residual solid waste. The resulting liquefied waste can be separated from said any residual solidified waste. See e.g., U.S. Pat. No. 5,709,796.

In one aspect, the compositions and methods of the invention are used for odor removal, odor prevention or odor reduction, e.g., in animal waste lagoons, e.g., on swine farms, in other agricultural, food or feed processing, in clothing and/or textile processing, cleaning or recycling, or other industrial processes.

The enzymes and methods for the conversion of biomass (e.g., lignocellulosic materials) to fuels (e.g., biofuels comprising, e.g., a bioalcohol such as bioethanol, biomethanol, biobutanol or biopropanol) can incorporate the treatment/recycling of municipal solid waste material, including waste obtained directly from a municipality or municipal solid waste that was previously land-filled and subsequently recovered, or sewage sludge, e.g., in the form of sewage sludge cake which contains substantial amounts of cellulosic material. Since sewage sludge cakes will normally not contain substantial amounts of recyclable materials (aluminum, glass, plastics, etc.), they can be directly treated with concentrated sulfuric acid (to reduce the heavy metal content of the cellulosic component of the waste) and processed in the ethanol production system. See, e.g., U.S. Pat. Nos. 6,267,309; 5,975,439.

Another exemplary method using enzymes of the invention for recovering organic and inorganic matter from waste material comprises sterilizing a solid organic matter and softening it by subjecting it to heat and pressure. This exemplary process may be carried out by first agitating waste material and then subjecting it to heat and pressure, which sterilizes it and softens the organic matter contained therein. In one aspect, after heating under pressure, the pressure may be suddenly released from a perforated chamber to forces the softened organic matter outwardly through perforations of the container, thus separating the organic matter from the solid inorganic matter. The softened sterilized, organic matter is then fermented in fermentation chamber, e.g., using enzymes of the invention, e.g., to form a mash. The mash may be subjected to further processing by centrifuge, distillation column and/or anaerobic digester to recover fuels such as ethanol and methane, and animal feed supplements. See, e.g., U.S. Pat. No. 6,251,643.

Enzymes of the invention can also be used in processes, e.g., pretreatments, to reduce the odor of an industrial waste, or a waste generated from an animal production facility, and the like. For example, enzymes of the invention can be used to treat an animal waste in a waste holding facility to enhance efficient degradation of large amounts of organic matter with reduced odor. The process can also include inoculation with sulfide-utilizing bacteria and organic digesting bacteria and lytic enzymes (in addition to an enzyme of the invention). See, e.g., U.S. Pat. No. 5,958,758.

Enzymes of the invention can also be used in mobile systems, e.g., batch type reactors, for bioremediation of aqueous, hazardous wastes, e.g., as described in U.S. Pat. No. 5,833,857. Batch type reactors can be large vessels having circulatory capability wherein bacteria (e.g., expressing an enzyme of the invention) are maintained in an efficient state by nutrients being feed into the reactor. Such systems can be used where effluent can be delivered to the reactor or the reactor is built into a waste water treatment system. Enzymes of the invention can also be used in treatment systems for use at small or temporary remote locations, e.g., portable, high volume, highly efficient, versatile waste water treatment systems.

The waste treatment processes of the invention can include the use of any combination of other enzymes such as other the lignocellulosic enzyme, e.g., glycosyl hydrolase, cellulase, endoglucanase, cellobiohydrolase, beta-glucosidase, xylanase, mannanse, β-xylosidase and/or arabinofuranosidase enzymes, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, phytases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, other cellobiohydrolases, and/or glucose oxidases.

Detergent Compositions

The invention provides detergent compositions comprising one or more polypeptides of the invention (e.g., enzymes having cellulase, endoglucanase, cellobiohydrolase, beta-glucosidase, xylanase, mannanse, β-xylosidase and/or arabinofuranosidase activity) and methods of making and using these compositions. The invention incorporates all methods of making and using detergent compositions, see, e.g., U.S. Pat. Nos. 6,413,928; 6,399,561; 6,365,561; 6,380,147. The detergent compositions can be a one and two part aqueous composition, a non-aqueous liquid composition, a cast solid, a granular form, a particulate form, a compressed tablet, a gel and/or a paste and a slurry form. The invention also provides methods capable of a rapid removal of gross food soils, films of food residue and other minor food compositions using these detergent compositions. Enzymes of the invention can facilitate the removal of starchy stains by means of catalytic hydrolysis of the starch polysaccharide. Enzymes of the invention can be used in dishwashing detergents in textile laundering detergents.

The actual active enzyme content depends upon the method of manufacture of a detergent composition and is not critical, assuming the detergent solution has the desired enzymatic activity. In one aspect, the amount of glucosidase present in the final solution ranges from about 0.001 mg to 0.5 mg per gram of the detergent composition. The particular enzyme chosen for use in the process and products of this invention depends upon the conditions of final utility, including the physical product form, use pH, use temperature, and soil types to be degraded or altered. The enzyme can be chosen to provide optimum activity and stability for any given set of utility conditions. In one aspect, the polypeptides of the present invention are active in the pH ranges of from about 4 to about 12 and in the temperature range of from about 20° C. to about 95° C. The detergents of the invention can comprise cationic, semi-polar nonionic or zwitterionic surfactants; or, mixtures thereof.

Enzymes of the present invention (e.g., enzymes having cellulase, endoglucanase, cellobiohydrolase, beta-glucosidase, xylanase, mannanse, β-xylosidase and/or arabinofuranosidase activity) can be formulated into powdered and liquid detergents having pH between 4.0 and 12.0 at levels of about 0.01 to about 5% (preferably 0.1% to 0.5%) by weight. These detergent compositions can also include other enzymes such as known proteases, cellulases, lipases or endoglycosidases, and/or glucose oxidases, as well as builders and stabilizers. The addition of enzymes of the invention to conventional cleaning compositions does not create any special use limitation. In other words, any temperature and pH suitable for the detergent is also suitable for the present compositions as long as the pH is within the above range, and the temperature is below the described enzyme's denaturing temperature. In addition, the polypeptides of the invention can be used in a cleaning composition without detergents, again either alone or in combination with builders and stabilizers.

The present invention provides cleaning compositions including detergent compositions for cleaning hard surfaces, detergent compositions for cleaning fabrics, dishwashing compositions, oral cleaning compositions, denture cleaning compositions, and contact lens cleaning solutions.

In one aspect, the invention provides a method for washing an object comprising contacting the object with a polypeptide of the invention under conditions sufficient for washing. A polypeptide of the invention may be included as a detergent additive. The detergent composition of the invention may, for example, be formulated as a hand or machine laundry detergent composition comprising a polypeptide of the invention. A laundry additive suitable for pre-treatment of stained fabrics can comprise a polypeptide of the invention. A fabric softener composition can comprise a polypeptide of the invention. Alternatively, a polypeptide of the invention can be formulated as a detergent composition for use in general household hard surface cleaning operations. In alternative aspects, detergent additives and detergent compositions of the invention may comprise one or more other enzymes such as a protease, a lipase, a cutinase, another glucosidase, a carbohydrase, another cellulase, a pectinase, a mannanase, an arabinase, a galactanase, a xylanase, an oxidase, e.g., a lactase, and/or a peroxidase, and/or glucose oxidase. The properties of the enzyme(s) of the invention are chosen to be compatible with the selected detergent (i.e. pH-optimum, compatibility with other enzymatic and non-enzymatic ingredients, etc.) and the enzyme(s) is present in effective amounts. In one aspect, enzymes of the invention are used to remove malodorous materials from fabrics. Various detergent compositions and methods for making them that can be used in practicing the invention are described in, e.g., U.S. Pat. Nos. 6,333,301; 6,329,333; 6,326,341; 6,297,038; 6,309,871; 6,204,232; 6,197,070; 5,856,164.

The detergents and related processes of the invention can also include the use of any combination of other enzymes such as tryptophanases or tyrosine decarboxylases, laccases, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, other cellobiohydrolases, and/or glucose oxidases.

Treating Fabrics and Textiles

The invention provides methods of treating fabrics and textiles using one or more polypeptides of the invention, e.g., enzymes having cellulase, endoglucanase, cellobiohydrolase, beta-glucosidase, xylanase, mannanse, β-xylosidase and/or arabinofuranosidase activity. The polypeptides of the invention can be used in any fabric-treating method, which are well known in the art, see, e.g., U.S. Pat. No. 6,077,316. For example, in one aspect, the feel and appearance of a fabric is improved by a method comprising contacting the fabric with an enzyme of the invention in a solution. In one aspect, the fabric is treated with the solution under pressure.

In one aspect, the enzymes of the invention are applied during or after the weaving of textiles, or during the desizing stage, or one or more additional fabric processing steps. During the weaving of textiles, the threads are exposed to considerable mechanical strain. Prior to weaving on mechanical looms, warp yarns are often coated with sizing starch or starch derivatives in order to increase their tensile strength and to prevent breaking. The enzymes of the invention can be applied to remove these sizing starch or starch derivatives. After the textiles have been woven, a fabric can proceed to a desizing stage. This can be followed by one or more additional fabric processing steps. Desizing is the act of removing size from textiles. After weaving, the size coating must be removed before further processing the fabric in order to ensure a homogeneous and wash-proof result. The invention provides a method of desizing comprising enzymatic hydrolysis of the size by the action of an enzyme of the invention.

The enzymes of the invention (e.g., enzymes having cellulase, endoglucanase, cellobiohydrolase, beta-glucosidase, xylanase, mannanse, β-xylosidase and/or arabinofuranosidase activity) can be used to desize fabrics, including cotton-containing fabrics, as detergent additives, e.g., in aqueous compositions. The invention provides methods for producing a stonewashed look on indigo-dyed denim fabric and garments. For the manufacture of clothes, the fabric can be cut and sewn into clothes or garments, which is afterwards finished. In particular, for the manufacture of denim jeans, different enzymatic finishing methods have been developed. The finishing of denim garment normally is initiated with an enzymatic desizing step, during which garments are subjected to the action of amylolytic enzymes in order to provide softness to the fabric and make the cotton more accessible to the subsequent enzymatic finishing steps. The invention provides methods of finishing denim garments (e.g., a “bio-stoning process”), enzymatic desizing and providing softness to fabrics using the Enzymes of the invention. The invention provides methods for quickly softening denim garments in a desizing and/or finishing process.

The invention also provides disinfectants comprising enzymes of the invention (e.g., enzymes having cellulase, endoglucanase, cellobiohydrolase, beta-glucosidase, xylanase, mannanse, β-xylosidase and/or arabinofuranosidase activity).

The fabric or textile treatment processes of the invention can also include the use of any combination of other enzymes such as tryptophanases or tyrosine decarboxylases, laccases, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, other cellobiohydrolases, and/or glucose oxidases.

Paper or Pulp Treatment

The enzymes of the invention (e.g., enzymes having cellulase, endoglucanase, cellobiohydrolase, beta-glucosidase, xylanase, mannanse, β-xylosidase and/or arabinofuranosidase activity) can be in paper or pulp treatment or paper deinking. For example, in one aspect, the invention provides a paper treatment process using enzymes of the invention. In one aspect, the enzymes of the invention can be used to modify starch in the paper thereby converting it into a liquefied form. In another aspect, paper components of recycled photocopied paper during chemical and enzymatic deinking processes. In one aspect, Enzymes of the invention can be used in combination with other enzymes, including other cellulases (including other endoglucanases, cellobiohydrolases and/or beta-glucosidases). The wood, wood waste, paper, paper product or pulp can be treated by the following three processes: 1) disintegration in the presence of an enzyme of the invention, 2) disintegration with a deinking chemical and an enzyme of the invention, and/or 3) disintegration after soaking with an enzyme of the invention. The recycled paper treated with an enzyme of the invention can have a higher brightness due to removal of toner particles as compared to the paper treated with just cellulase. While the invention is not limited by any particular mechanism, the effect of an enzyme of the invention may be due to its behavior as surface-active agents in pulp suspension.

The invention provides methods of treating paper and paper pulp using one or more polypeptides of the invention. The polypeptides of the invention can be used in any paper- or pulp-treating method, which are well known in the art, see, e.g., U.S. Pat. Nos. 6,241,849; 6,066,233; 5,582,681. For example, in one aspect, the invention provides a method for deinking and decolorizing a printed paper containing a dye, comprising pulping a printed paper to obtain a pulp slurry, and dislodging an ink from the pulp slurry in the presence of an enzyme of the invention (other enzymes can also be added). In another aspect, the invention provides a method for enhancing the freeness of pulp, e.g., pulp made from secondary fiber, by adding an enzymatic mixture comprising an enzyme of the invention (can also include other enzymes, e.g., pectinase enzymes) to the pulp and treating under conditions to cause a reaction to produce an enzymatically treated pulp. The freeness of the enzymatically treated pulp is increased from the initial freeness of the secondary fiber pulp without a loss in brightness.

The paper, wood, wood waste, or pulp treatment or recycling processes of the invention can also include the use of any combination of other enzymes such as tryptophanases or tyrosine decarboxylases, laccases, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, other cellobiohydrolases, and/or glucose oxidase.

Repulping: Treatment of Lignocellulosic Materials

The invention also provides a method for the treatment of lignocellulosic fibers, wherein the fibers are treated with a polypeptide of the invention (e.g., enzymes having cellulase, endoglucanase, cellobiohydrolase, beta-glucosidase, xylanase, mannanse, β-xylosidase and/or arabinofuranosidase activity), in an amount which is efficient for improving the fiber properties. The enzymes of the invention may also be used in the production or recycling of lignocellulosic materials such as pulp, paper and cardboard, from starch reinforced waste paper and cardboard, especially where repulping or recycling occurs at pH above 7 and where the enzymes of the invention can facilitate the disintegration of the waste material through degradation of the reinforcing starch. The enzymes of the invention can be useful in a process for producing a papermaking pulp from starch-coated printed paper. The process may be performed as described in, e.g., WO 95/14807. An exemplary process comprises disintegrating the paper to produce a pulp, treating with a starch-degrading enzyme before, during or after the disintegrating, and separating ink particles from the pulp after disintegrating and enzyme treatment. See also U.S. Pat. No. 6,309,871 and other US patents cited herein. Thus, the invention includes a method for enzymatic deinking of recycled paper pulp, wherein the polypeptide is applied in an amount which is efficient for effective de-inking of the fiber surface.

Brewing and Fermenting

The invention provides methods of brewing (e.g., fermenting) beer comprising an enzyme of the invention, e.g., enzymes having cellulase, endoglucanase, cellobiohydrolase, beta-glucosidase, xylanase, mannanse, β-xylosidase and/or arabinofuranosidase activity. In one exemplary process, starch-containing raw materials are disintegrated and processed to form a malt. An enzyme of the invention is used at any point in the fermentation process. For example, enzymes of the invention can be used in the processing of barley malt. The major raw material of beer brewing is barley malt. This can be a three stage process. First, the barley grain can be steeped to increase water content, e.g., to around about 40%. Second, the grain can be germinated by incubation at 15-25° C. for 3 to 6 days when enzyme synthesis is stimulated under the control of gibberellins. During this time enzyme levels rise significantly. In one aspect, enzymes of the invention are added at this (or any other) stage of the process. The action of the enzyme results in an increase in fermentable reducing sugars. This can be expressed as the diastatic power, DP, which can rise from around 80 to 190 in 5 days at 12° C.

Enzymes of the invention can be used in any beer producing process, as described, e.g., in U.S. Pat. Nos. 5,762,991; 5,536,650; 5,405,624; 5,021,246; 4,788,066.

Increasing the Flow of Production Fluids from a Subterranean Formation

The invention also includes a method using an enzyme of the invention (e.g., enzymes having cellulase, endoglucanase, cellobiohydrolase, beta-glucosidase, xylanase, mannanse, β-xylosidase and/or arabinofuranosidase activity), wherein the method increases the flow of production fluids from a subterranean formation by removing viscous, starch-containing, damaging fluids formed during production operations; these fluids can be found within the subterranean formation which surrounds a completed well bore. Thus, this method of the invention results in production fluids being able to flow from the well bore. This method of the invention also addresses the problem of damaging fluids reducing the flow of production fluids from a formation below expected flow rates. In one aspect, the invention provides for formulating an enzyme treatment (using an enzyme of the invention) by blending together an aqueous fluid and a polypeptide of the invention; pumping the enzyme treatment to a desired location within the well bore; allowing the enzyme treatment to degrade the viscous, starch-containing, damaging fluid, whereby the fluid can be removed from the subterranean formation to the well surface; and wherein the enzyme treatment is effective to attack the alpha glucosidic linkages in the starch-containing fluid.

The subterranean formation enzyme treatment processes of the invention can also include the use of any combination of other enzymes such as tryptophanases or tyrosine decarboxylases, laccases, catalases, laccases, other cellulases, endoglycosidases, endo-beta-1,4-laccases, amyloglucosidases, other glucosidases, glucose isomerases, glycosyltransferases, lipases, phospholipases, lipooxygenases, beta-laccases, endo-beta-1,3(4)-laccases, cutinases, peroxidases, amylases, glucoamylases, pectinases, reductases, oxidases, decarboxylases, phenoloxidases, ligninases, pullulanases, arabinanases, hemicellulases, mannanases, xylolaccases, xylanases, pectin acetyl esterases, rhamnogalacturonan acetyl esterases, proteases, peptidases, proteinases, polygalacturonases, rhamnogalacturonases, galactanases, pectin lyases, transglutaminases, pectin methylesterases, other cellobiohydrolases, and/or glucose oxidase.

Pharmaceutical Compositions and Dietary Supplements

The invention also provides pharmaceutical compositions and dietary supplements (e.g., dietary aids) comprising a cellulase of the invention (e.g., enzymes having endoglucanase, cellobiohydrolase, beta-glucosidase, xylanase, mannanse, β-xylosidase and/or arabinofuranosidase activity). The cellulase activity comprises endoglucanase, cellobiohydrolase, beta-glucosidase, xylanase, mannanse, β-xylosidase and/or arabinofuranosidase activity. In one aspect, the pharmaceutical compositions and dietary supplements (e.g., dietary aids) are formulated for oral ingestion, e.g., to improve the digestibility of foods and feeds having a high cellulose or lignocellulosic component.

Periodontal treatment compounds can comprise an enzyme of the invention, e.g., as described in U.S. Pat. No. 6,776,979. Compositions and methods for the treatment or prophylaxis of acidic gut syndrome can comprise an enzyme of the invention, e.g., as described in U.S. Pat. No. 6,468,964.

In another aspect, wound dressings, implants and the like comprise antimicrobial (e.g., antibiotic-acting) enzymes, including an enzyme of the invention (including, e.g., exemplary sequences of the invention). Enzymes of the invention can also be used in alginate dressings, antimicrobial barrier dressings, burn dressings, compression bandages, diagnostic tools, gel dressings, hydro-selective dressings, hydrocellular (foam) dressings, hydrocolloid dressings, I.V dressings, incise drapes, low adherent dressings, odor absorbing dressings, paste bandages, post operative dressings, scar management, skin care, transparent film dressings and/or wound closure. Enzymes of the invention can be used in wound cleansing, wound bed preparation, to treat pressure ulcers, leg ulcers, burns, diabetic foot ulcers, scars, IV fixation, surgical wounds and minor wounds. Enzymes of the invention can be used to in sterile enzymatic debriding compositions, e.g., ointments. In various aspects, the cellulase is formulated as a tablet, gel, pill, implant, liquid, spray, powder, food, feed pellet or as an encapsulated formulation.

Biodefense Applications

In other aspects, enzymes and antibodies of this invention, including enzymes having lignocellulosic activity, including polypeptides having cellulase, endoglucanase, cellobiohydrolase, beta-glucosidase, xylanase, mannanse, β-xylosidase and/or arabinofuranosidase activity, can be used in biodefense; e.g., for the destruction of spores or microorganisms, e.g., bacteria, fungi, yeast, etc., comprising a lignocellulosic material or any biologic polymer susceptible to hydrolysis by a polypeptide of this invention. Use of enzymes and antibodies of this invention, including enzymes having lignocellulosic activity, including polypeptides having cellulase, endoglucanase, etc. activity, in biodefense applications offers a significant benefit, in that they can be very rapidly manufactured and/or developed against any currently unknown or biological warfare agents of the future. In addition, enzymes having lignocellulosic activity, including polypeptides having cellulase, etc. activity, can be used for decontamination of affected environments or materials, including clothing, or individuals. Thus, in aspect, the invention provides a biodefense or bio-detoxifying agent(s), or disinfecting agent, comprising a polypeptide having lignocellulosic activity, including polypeptides having cellulase, etc. activity, wherein the polypeptide comprises a sequence of the invention (including, e.g., exemplary sequences of the invention), or a polypeptide encoded by a nucleic acid of the invention (including, e.g., exemplary sequences of the invention), and methods of making and using them. In one aspect, the polypeptide has activity comprising endoglucanase, cellobiohydrolase, beta-glucosidase, xylanase, mannanse, β-xylosidase and/or arabinofuranosidase activity.

### REFERENCE LIST

1. Sambrook, J. and Russell, D. W. 2001. Molecular Cloning: A Laboratory Manual. Third Edition. Cold Spring Harbor Laboratory Press, New York.

2. Benhar, I. Biotechnological applications of phage and cell display. Biotechnology Advances 19, 1-13. 2001.

3. Carbohydrate-Active Enzymes CAZy server on the interne; citation is Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12.

4. Felix, C. R. and L. G. Ljungdahl. 1993. The cellulosome: the exocellular organelle of Clostridium. Annu. Rev. Microbiol 47:791-819.:791-819.

5. Gray (2001) Rapid evolution of reversible denaturation and elevated melting temperature in a microbial haloalkane dehalogenase. Advanced Synthesis and Catalysis 343:607-617.

6. Guttman (1996) High-resolution capillary gel electrophoresis of reducing oligosaccharides labeled with 1-aminopyrene-3,6,8-trisulfonate. Anal. Biochem 233:234-242.

7. Harjunpaa (1996) Cello-oligosaccharide hydrolysis by cellobiohydrolase II from Trichoderma reesei. Association and rate constants derived from an analysis of progress curves. Eur. J Biochem 240:584-591.

8. Himmel (1999) Cellulase for commodity products from cellulosic biomass. Curr. Opin. Biotechnol 10:358-364.

9. Kerr, R. A. 1998. GEOLOGY:The Next Oil Crisis Looms Large—and Perhaps Close. Science 281:1128.

10. Kerr, R. A. 2000. OIL OUTLOOK:USGS Optimistic on World Oil Prospects. Science 289:237.

11. King (1997) Expression cloning in the test tube. Science 277:973-974.

12. Kuritz, T. 1999. An easy colorimetric assay for screening and qualitative assessment of deiodination and dehalogenation by bacterial cultures. Lett. Appl Microbiol 28:445-447.

13. Lundberg (1993) The use of selection in recovery of transgenic targets for mutation analysis. Mutat. Res. 301:99-105.

14. MacKenzie (1998) Crystal structure of the family 7 endoglucanase I (Cel7B) from Humicola insolens at 2.2 A resolution and identification of the catalytic nucleophile by trapping of the covalent glycosyl-enzyme intermediate. Biochem J 335:409-416.

15. Richardson (2002) A novel, high performance enzyme for starch liquefaction. Discovery and optimization of a low pH, thermostable alpha-amylase. J Biol Chem 277:26501-26507.

16. Sakon (1997) Structure and mechanism of endo/exocellulase E4 from Thermomonospora fusca. Nat. Struct. Biol 4:810-818.

17. Short (1988) Lambda ZAP: a bacteriophage lambda expression vector with in vivo excision properties. Nucleic Acids Res. 16:7583-7600.

18. Snustad (1988) Maize glutamine synthetase cDNAs: isolation by direct genetic selection in Escherichia coli. Genetics 120:1111-1123.

19. Varrot (1999) Crystal structure of the catalytic core domain of the family 6 cellobiohydrolase II, Cel6A, from Humicola insolens, at 1.92 A resolution. Biochem J 337:297-304.

20. Yano (1998) Directed evolution of an aspartate aminotransferase with new substrate specificities. Proc. Natl. Acad. Sci U.S.A 95:5511-5515.

21. Zverlov (2002) A newly described cellulosomal cellobiohydrolase, CelO, from Clostridium thermocellum: investigation of the exo-mode of hydrolysis, and binding capacity to crystalline cellulose. Microbiology 148:247-255.

The following examples are offered to illustrate, but not to limit the claimed invention.

### Examples

### Example 1

### Exemplary Screening Protocol Using GIGAMATRIX™ Screening

The invention provides methods for screening for enzymes having lignocellulosic activity. These described methods can also be used to determine if an enzyme has the requisite activity and is with the scope of the claimed invention. In one aspect, the methods of the invention use Verenium Corporation's proprietary GIGAMATRIX™ platform; see, e.g., PCT Patent Publication No. WO 01/38583; U.S. patent application no. 20050046833; 20020080350; U.S. Pat. No. 6,918,738; Design U.S. Patent No. D480,814. For example, in one aspect, GIGAMATRIX™ is used in methods to determine if a polypeptide has a lignocellulosic activity and is within the scope of the invention, or, to identify and isolate a polypeptide having lignocellulosic activity, e.g., a polypeptide having a glycosyl hydrolase, cellulase, endoglucanase, cellobiohydrolase and/or β-glucosidase (beta-glucosidase) activity.

A GIGAMATRIX™ platform can include an ultra-high throughput screen based on a 100,000 well microplate with the dimensions of a conventional 96 well plate. While in this example, the GIGAMATRIX™ screen implemented use of two (2) substrates—Methyl-umbelliferyl cellobioside (MUC) and methylumbelliferyl lactoside (MUL), any substrate specific for or determinative of any lignocellulosic activity can be used, including substrates for cellulase, endoglucanase, cellobiohydrolase and/or β-glucosidase.

Phagemid versions of different clones can be screened because the substrate diffuses into cells and fluorescence was thought to be more easily detectable. A host strain lacking, beta-galactosidase can be used in order to decrease activity on the lactoside substrate. The lactoside substrate can result in fewer hits and can be deemed more specific than the cellobiose substrate. In addition, the lactoside substrate can result in fewer beta-glucosidase hits. A secondary screening can consist of plating the clones on agar plates and then colony picking into 384 well plates containing media and MUL. Active clones against MUL are differentiated from a background of inactive clones. Individual clones can then be grown overnight and fluorescence measured. The most active hits can then be picked for sequencing.

### Characterization Enzyme and Substrate Activity

The hits discovered in the GIGAMATRIX™ screen can first be screened against cellohexaose to determine action pattern on a cellulose oligomer. Clones can be grown overnight in TB media containing antibiotic, cells can then be lysed and lysates clarified by centrifugation. Subclones can be grown to an OD600=0.5 induced with an appropriate inducer and then grown an additional 3 h before lysing the cells and clarifying the lysate. Genomic clones will generally have less activity than a subclone, but are a more facile way of assessing activity in a large range of clones. Initial studies can be performed using thin layer chromatography (TLC) for endpoint reactions usually run for 24 h. Enzymes can also be tested on phosphoric acid swollen cellulose (PASC), which is crystalline cellulose that is made more amorphous through swelling by acid treatment.

Cellulases which are active against PASC, can also release cellobiose as well as cellotriose and/or glucose. The clones from the GIGAMATRIX™ discovery effort can is be also tested against PASC and on cellulosic substrates such as cellohexaose (e.g., Seikagaku, Japan). Thin layer chromatography (TLC) experiments can be use to show that clones are able to hydrolyze the cellohexaose. Of these clones, some are able to generate glucose as the final product. Several enzymes can produce cellobiose and/or larger fragments, but when the exact nature of the product pattern can not be discerned from the TLC experiments, a capillary electrophoresis (CE) method can also be used.

### Example 2

### Sequence Based Discovery

The invention provides methods for identifying and isolating biomass—(e.g., bagasse, corn fiber)—degrading enzymes, including polypeptides having a lignocellulolytic activity, e.g., a glycosyl hydrolase, a cellulase, an endoglucanase, a cellobiohydrolase, a beta-glucosidase, a xylanase, a mannanse, a xylosidase (e.g., a β-xylosidase) and/or an arabinofuranosidase activity, using nucleic acid sequences of the invention, e.g., as hybridization probes and/or as amplification (e.g., PCR) primers.

The invention provides amplification primer pairs for amplifying (e.g., by PCR) nucleic acids (including transcripts or genes) encoding a polypeptide having a lignocellulosic activity, e.g., a glycosyl hydrolase, cellulase, endoglucanase, glucosidase (beta-glucosidase), xylanase, xylosidase (e.g., β-xylosidase) and/or arabinofuranosidase activity, or can hydrolyze (degrade) soluble cellooligsaccharides and arabinoxylan oligomers into monomer xylose, arabinose and glucose, wherein the primer pair is capable of amplifying a nucleic acid comprising a sequence of the invention, or fragments or subsequences thereof. One or each member of the amplification primer sequence pair can comprise an oligonucleotide comprising at least about 10 to 50, or more, consecutive bases of the sequence, or about 10, 11, 12, 13, 14, 15, 16, 17, 18, 19, 20, 21, 22, 23, 24, 25, 26, 27, 28, 29, 30, 31, 32, 33, 34, 35, 36 or more consecutive bases of the sequence. The invention provides amplification primer pairs, wherein the primer pair comprises a first member having a sequence as set forth by about the first (the 5′) 12, 13, 14, 15, 16, 17, 18, 19, 20, 21, 22, 23, 24, 25, 26, 27, 28, 29, 30, 31, 32, 33, 34, 35, 36 or more residues of a nucleic acid of the invention, and a second member having a sequence as set forth by about the first (the 5′) 12, 13, 14, 15, 16, 17, 18, 19, 20, 21, 22, 23, 24, 25, 26, 27, 28, 29, 30, 31, 32, 33, 34, 35, 36 or more residues of the complementary strand of the first member.

### Example 3

### Genetic Engineering of and Screening for Lignocellulosic Enzymes

This example describes an exemplary protocol for the genetic engineering of an enzyme of the invention. The engineered, or “optimized”, enzyme of the invention can be used in the conversion of biomass (e.g., bagasse, corn fiber) to monosaccharides, fuels and/or chemicals or other useful products; e.g., for making effective and sustainable alternatives to petroleum-based products. The engineered, or “optimized”, enzyme of the invention can be expressed in organisms (e.g., microorganisms, such as bacteria) for its participation in chemical cycles involving natural biomass conversion. In one aspect, this engineered, or “optimized”, enzyme of the invention is used in “enzyme ensembles” for the efficient depolymerization of cellulosic and hemicellulosic polymers to metabolizable carbon moieties. As discussed above, the invention provides methods for discovering and implementing the most effective of enzymes to enable these important new “biomass conversion” and alternative energy industrial processes.

Using metagenomic discovery and a non-stochastic method of directed evolution (called “DIRECTEVOLUTION®, as described, e.g., in U.S. Pat. No. 6,939,689, which includes GENE SITE SATURATION MUTAGENESIS (or GSSM) (as discussed above, see also U.S. Pat. Nos. 6,171,820 and 6,579,258) and Tunable GeneReassembly (TGR) (see, e.g., U.S. Pat. No. 6,537,776) technologies. These technologies can be used for the discovery and optimization of an enzyme component for lignocellulosic biomass material (e.g., cellulose) reduction (e.g., hydrolysis) to monosaccharides (e.g., glucose), cellobiohydrolase and other carbohydrates.

In one embodiment, an enzyme discovery screen can be implemented using Verenium Corporation's GIGAMATRIX™ high throughput expression screening platform (discussed above) to identify enzymes; for example, to identify cellobiohydrolases using methylumbelliferyl cellobioside as a substrate. Hits can be characterized for activity against AVICEL® Microcrystalline Cellulose (MCC) (FMC Corporation, Philadelphia, Pa.).

In one aspect, an enzyme can be chosen as a candidate for optimization using GENE SITE SATURATION MUTAGENESIS (or GSSM) technology. In one embodiment, before performing GSSM evolution, the signal sequence, if present, can be removed and a starting methionine added. As discussed above, GSSM technology can rapidly mutate all amino acids in the protein to the 19 other amino acids in a sequential fashion. Mutants can be screened using a fiber-based assay and potential upmutants representing single amino acid changes can be identified. These upmutants can be combined into a new library representing combinations of the upmutants. This library can be screened resulting in identification of several candidate enzymes for commercialization.

Blending of Upmutants

Using gene reassembly (Tunable GeneReassembly (TGR)) technology, GSSM upmutants (enzyme-encoding sequence variants) can be “blended” (mixed together to achieve an optimal result) in order to construct an enzyme with a desired activity or trait; and then screening (e.g., GSSM) can be used to identify candidate(s) with the best desired activity or trait (e.g., thermotolerance). Activity assays can be the same as for the GSSM screening except reactions can be further diluted to account for increased activity of upmutants over the wildtype enzyme.

### Example 4

### Enzyme Mixtures, or “Cocktails” for Processing/Converting Biomass

The invention provides novel combinations, or mixtures, or “cocktails”, of enzymes for processing lignocellulosic-comprising biomass, e.g., bagasse or corn fiber, to useable products, for example, to lignin, or monosaccharides such as glucose, which can then be processed into ethanol. This example describes enzyme mixtures, or “cocktails”, of the invention to digest biomass, e.g. bagasse, into fermentable sugars, and their development. In one aspect, the enzyme mixtures, or “cocktails” comprise at least one exemplary enzyme of the invention. A mixture (“ensemble” or “cocktail”) of the invention can also comprise any other enzyme, e.g., a glucose oxidase, a phosphorylase, and amidase, etc., and the like.

In one embodiment, the enzyme mixtures, or “cocktails”, of the invention are used to hydrolyze lignocellulosic material, e.g., cellulose or any β1,4-linked glucose moieties and/or hemicellulose or any branched polymer comprising a β-1,4-linked xylose backbone with branches of arabinose, galactose, mannose, glucuronic acid, and/or linkages to lignin, e.g., via ferulic acid ester groups. Thus, in various aspects, the methods and compositions of the invention address the complexity and problems of digestion of hemicellulose to monomer sugars due to the variability of sugars and linkages.

### Exemplary Combinatorial Enzyme Screening Protocol

1. Prepare Enzyme Panel Plates
   1.1. Resuspend lyophilized protein powder in 50% glycerol to a concentration of 25 mg protein/mL
   1.2. Array 200 μL of each enzyme on a 96-well plate
   1.3. Store at −20° C.
2. Prepare Enzyme Cocktail Plates
   2.1. Prepare 11.11× solution of constant enzymes in diH₂O (0.1 mg/mL total enzyme concentration)
   2.2. Dispense 27 μL into wells of two 96-well plates (High Dose and Low Dose)
   2.3. Transfer 3 μL from Enzyme Panel plate to the High Dose plate. Mix well
   2.4. Transfer 3 μL from High Dose plate to the Low Dose plate. Mix well
3. Prepare Solution of Buffered Substrate
   3.1. Want pH controlled buffer, sodium azide, and xylanase at 1.11× concentration (55.56 mM, 5.56 mM, and 0.32 mg/mL, respectively)
   3.2. Want substrate at 1× concentration of 0.1% cellulose (approximately 2% pretreated ground bagasse, depending on cellulose content of substrate batch)
4. Prepare Stop Plates
   4.1. Make a solution of 150 mM sodium carbonate buffer, pH10
   4.2. Dispense 60 pl. per well into a 384-well plate
5. Prepare Digest Plates
   5.1. Dispense 180 μL of buffered substrate per well into two 96-well plates (High and Low Dose plates)
   5.2. Transfer 20 μL from High Dose cocktail plate to High Dose digest plate, pipette to mix
      5.2.1. Allow substrate to settle briefly and transfer 20 μL from Digest Plate to the Stop Plate for T=0 timepoint
   5.3. Repeat steps 5.2 for Low Dose cocktail plate
   5.4. Seal Digest Plates and incubate at 37° C. for 4 hours
   5.5. Spin Digest Plates at 3000 rpm for 1 minute to bring supernatant to the plate bottom
   5.6. Transfer 20 μL from Digest Plate to Stop Plate for T=4 hr timepoint
   5.7. Add glucose and cellobiose standards to Stop Plate
6. β-glucosidase digest
   6.1. Prepare a solution of β-glucosidase (approximately 35 mg/mL) in 125 mM sodium phosphate buffer, pH 7.0 and dispense 35 μL per well into a 384-well plate
   6.2. Using the APRICOT™ system (Process Analysis and Automation Ltd, Hampshire, UK), transfer 4 μL from the Stop Plate to the β-glucosidase plate; Incubate at room temperature for 3 hrs
7. Glucose Oxidase (GO) Assay
   7.1. Prepare 2× GO assay solution
      7.1.1. 100 mM pH7.4 sodium phosphate buffer, 2 U/mL glucose oxidase, 0.2 U/mL Horseradish peroxidase, 0.1 mM Amplex Red. Mix well
   7.2. Immediately add 40 μL per well to the β-glucosidase plate; Incubate at room temperature for 25-30 minutes
   7.3. Read 530 nm/595 nm excitation/emission on spectrophotometer.

Assays for the individual screening of enzyme activity, including an exemplary large scale enzyme digestibility assay, are described below in Example 5.

The following Table 4 summarizes several exemplary enzyme “cocktails” or mixtures of the invention, and their characterization:

TABLE 4

| | | | % conversion after 4 hours | | | | |
|---|---|---|---|---|---|---|---|
| | | | 96-well plate | small scale | large scale | | |
| enzyme 1 | enzyme 2 | enzyme 3 | (normalized¹) | rxns | rxns | optimal ratio | Comments |
| *SEQ ID NO: 34 | *SEQ ID NO: 360 | #SEQ ID NO: 214 | 22.0% | 20.55% | 11.2% | 33:33:33 | |
| SEQ ID NO: 360 | #SEQ ID NO: 90 | *SEQ ID NO: 358 | 23.2% | 32.21% | 6.5% | 25:25:50 | |
| *SEQ ID NO: 360 | #SEQ ID NO: 90 | #SEQ ID NO: 428 | 19.9% | | 0.8% | 60:10:30 | |
| *SEQ ID NO: 360 | #SEQ ID NO: 90 | *SEQ ID NO: 401 | 19.7% | 34.53% | 6.9% | 42:40:18 | |
| *SEQ ID NO: 360 | #SEQ ID NO: 426 | #SEQ ID NO: 366 | 16.0% | 20.10% | 9.9% | 25:25:50 | |
| *SEQ ID NO: 360 | #SEQ ID NO: 426 | #SEQ ID NO: 134 | 15.8% | 17.53% | | 37:14:49 | |
| *SEQ ID NO: 360 | #SEQ ID NO: 426 | #SEQ ID NO: 214 | 19.0% | 21.26% | 3.6% | 53:11:36 | |
| *SEQ ID NO: 360 | #SEQ ID NO: 426 | #SEQ ID NO: 2 | 18.1% | 18.07% | | 50:25:25 | |
| *SEQ ID NO: 360 | *SEQ ID NO: 34 | #SEQ ID NO: 366 | 18.6% | 18.13% | | 42:40:18 | |
| *SEQ ID NO: 360 | #SEQ ID NO: 2 | *SEQ ID NO: 377 | 17.1% | | | | |
| *SEQ ID NO: 360 | #SEQ ID NO: 2 | *SEQ ID NO: 358 | 16.1% | | | | |
| #SEQ ID NO: 426 | #SEQ ID NO: 176 | #SEQ ID NO: 428 | 8.2% | 21.14% | 1.9% | 33:33:33 | all bacterial |
| #SEQ ID NO: 426 | #SEQ ID NO: 176 | #SEQ ID NO: 430 | 8.2% | 14.52% | | 25:50:25 | all bacterial |
| *SEQ ID NO: 34 | *SEQ ID NO: 371 | #SEQ ID NO: 168 | 19.1% | 23.31% | 8.5% | 60:10:30 | |
| *SEQ ID NO: 34 | *SEQ ID NO: 360 | #SEQ ID NO: 168 | 17.9% | | 10.3% | | |
| *SEQ ID NO: 34 | *SEQ ID NO: 282 | #SEQ ID NO: 90 | 18.6% | | 7.4% | | |
| *SEQ ID NO: 360 | *SEQ ID NO: 282 | #SEQ ID NO: 168 | 18.0% | | 10.6% | | |
| *SEQ ID NO: 360 | *SEQ ID NO: 36 | #SEQ ID NO: 90 | 17.8% | | | | |
| *SEQ ID NO: 34 | *SEQ ID NO: 282 | #SEQ ID NO: 168 | 17.3% | | 10.1% | | |
| *SEQ ID NO: 360 | *SEQ ID NO: 282 | #SEQ ID NO: 74 | 17.0% | | | | |
| *SEQ ID NO: 360 | *SEQ ID NO: 282 | #SEQ ID NO: 90 | 16.8% | | | | |
| *SEQ ID NO: 360 | *SEQ ID NO: 36 | #SEQ ID NO: 168 | 16.8% | | | | |
| **SEQ ID NO: 182 | *SEQ ID NO: 36 | ##SEQ ID NO: 40 | 23.3% | | | | |
| **SEQ ID NO: 182 | *SEQ ID NO: 36 | ##SEQ ID NO: 38 | 23.7% | | | | |
| **SEQ ID NO: 140 | *SEQ ID NO: 36 | ##SEQ ID NO: 38 | 23.6% | | | | |
| *SEQ ID NO: 34 | *SEQ ID NO: 358 | #SEQ ID NO: 168 | 19.6% | 24.98% | 14.5% | 67:7:26 | DVSA #1 |

¹Percent conversion normalized to the average DVSA#1 conversion value
Enzymes Expressed in:
*Apergillus niger
#E. coli
**Streptomyces diversa
##Pichia pastoris

Additional enzyme “mixtures” or “cocktails” of the invention comprise the following several combinations of the exemplary enzymes SEQ ID NO:34; SEQ ID NO:360; SEQ ID NO:358; and SEQ ID NO:371. The following chart summarizes the results of the various exemplary mixtures' enzymatic activity under conditions comprising a 37° C. digestion on a 0.1% AVICEL® substrate, where the total enzyme dose was held constant at 20 mg/g cellulose:

| | 0 | 1.25 | 2.5 | 4 |
|---|---|---|---|---|
| BD | 0% | 4% | 5% | 6% |
| CD | 0% | 4% | 5% | 6% |
| DF | 0% | 4% | 5% | 6% |
| BB | 0% | 1% | 2% | 2% |
| CC | 0% | 1% | 2% | 2% |
| DD | 0% | 1% | 2% | 2% |
| FF | 0% | 2% | 2% | 3% |
| neg | 0% | 0% | 0% | 0% |

enzyme ID
B SEQ ID NO: 34
C SEQ ID NO: 360
D SEQ ID NO: 358
F SEQ ID NO: 371

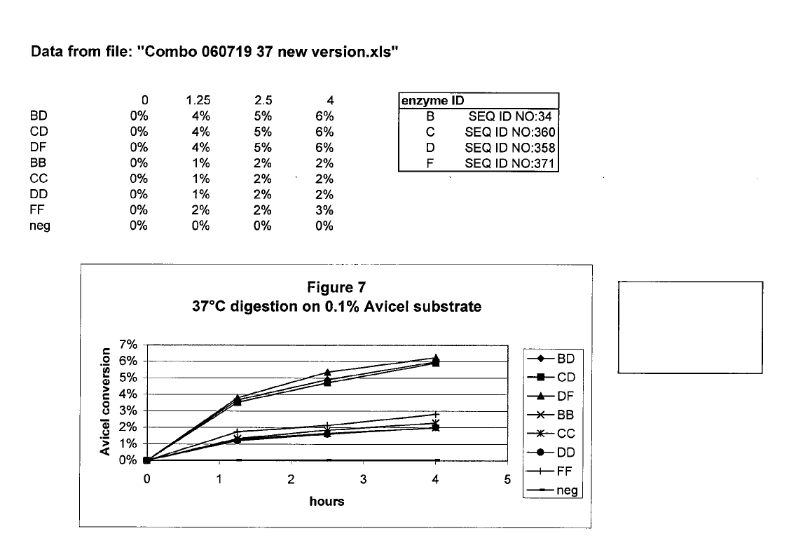

Data summarizing the results of the various exemplary mixtures' enzymatic activity under conditions comprising 37° C. digest on 0.1% AVICEL® substrate is illustrated in FIG. 7.

Additional enzyme “mixtures” or “cocktails” of the invention comprise the following several combinations of the exemplary enzymes SEQ ID NO:358; SEQ ID NO:360; SEQ ID NO:168; the following charts summarize the results of the various exemplary mixtures' enzymatic activity under conditions comprising a 37° C. digestion on a 0.1% AVICEL® substrate, where the total enzyme dose was held constant at 20 mg/g cellulose:

with 22419

| | conversion | stdev | conversion | stdev |
|---|---|---|---|---|
| AB | 0.4% | 0.1% | 2.7% | 0.5% |
| C + vector ctrl | | | 0.8% | 0.1% |
| AA | 0.3% | 0.0% | 1.4% | 0.0% |
| BB | 0.2% | 0.1% | 2.4% | 0.1% |

(See above)

| | | |
|---|---|---|
| A | 0.3% | 0.0% |
| B | 0.2% | 0.1% |
| C | 0.8% | 0.1% |
| A + B | 0.4% | 0.1% |
| B + C | 2.4% | 0.1% |
| A + C | 1.4% | 0.0% |
| A + B + C | 2.7% | 0.5% |

enzyme ID
A SEQ ID NO: 358 (encoded, e.g., by SEQ ID NO: 357)
B SEQ ID NO: 360 (encoded, e.g., by SEQ ID NO: 359)
C SEQ ID NO: 168 (encoded, e.g., by SEQ ID NO: 367)

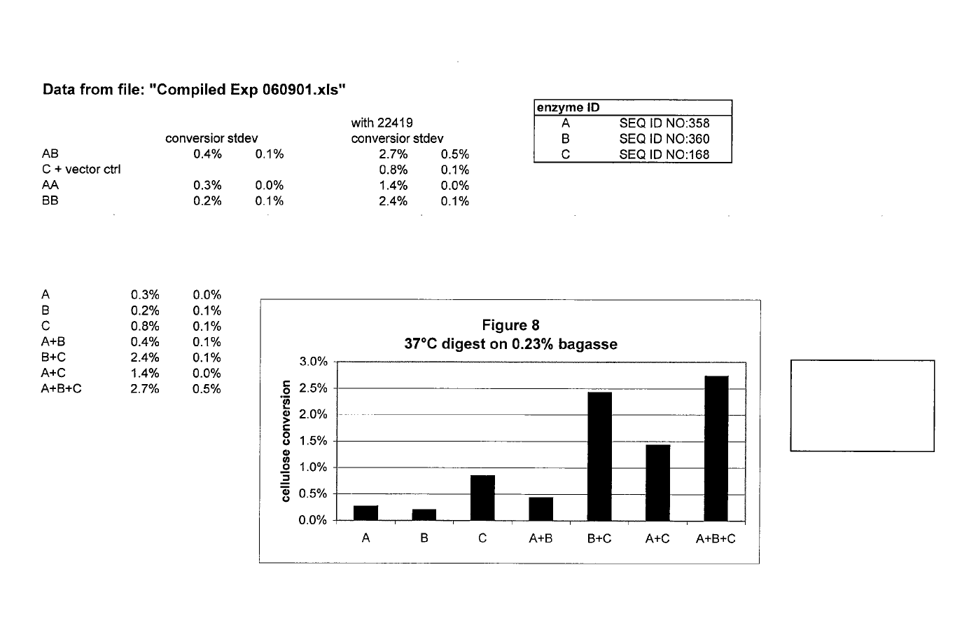

Data summarizing the results of the various exemplary mixtures' enzymatic activity under conditions comprising 37° C. digest on 0.23% bagasse is illustrated in FIG. 8.

Enzyme B and Enzyme C together have a synergistic effect: individually, Enzyme B gives 0.2% conversion and Enzyme C gives 0.8% conversion, so it would be expected that using them together would give 1% conversion, however, instead the Enzyme B and C combination gives a 2.4% conversion—clearly a synergistic effect. Other compositions of the invention comprising mixtures, or “cocktails” of the invention also can give a synergistic effect with regard to hydrolysis of a biomass, e.g., bagasse conversion, as described in this Example.

### Example 5

### Characterization of the Activity of Enzymes of the Invention

This example describes alternative exemplary screening protocols for the characterization and identification of enzymes of the invention. For example, this example describes how exemplary enzymes of the invention can be identified and used as lignocellulolytic enzymes for the hydrolysis of a biomass, e.g., plant biomass, such as bagasse or corn fiber (e.g., corn seed fiber). In one aspect, exemplary enzymes of the invention are used alone or in combination as glycosyl hydrolases, endoglucanases, cellobiohydrolases and/or β-glucosidases for, e.g., the treatment, e.g. saccharification, of cellulose or cellulose-comprising compositions, such as plant biomass, e.g., sugarcane bagasse, corn fiber or other plant waste material (such as a hay or straw, e.g., a rice straw or a wheat straw, or any the dry stalk of any cereal plant) or processing or agricultural byproduct.

Glucose Oxidase Assay for Quantifying Glucose

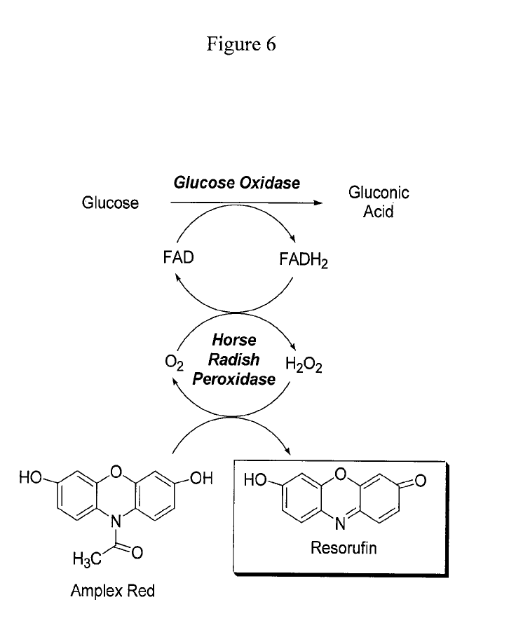

This exemplary protocol describes a glucose oxidase assay for quantifying glucose: a fluorescent enzyme-coupled assay to indirectly measure glucose concentration in complex mixtures (e.g. feedstocks, fiber samples, bagasse, corn fiber or other plant waste material or processing or agricultural byproduct, etc.). Glucose produced during enzymatic hydrolysis of carbohydrates is oxidized with glucose oxidase: this oxidation is coupled to a peroxidase and a fluorescent dye, and the resulting fluorescence is quantified by comparing against a glucose standard curve. A schematic of the assay, as illustrated in FIG. 6, shows that FAD is bound to the glucose oxidase, and is not an added component.

The following materials are needed for this exemplary assay:
Powdered Glucose Oxidase (e.g., A. niger Sigma Cat#G 7141);
Horseradish Peroxidase (liquid: e.g., Sigma Cat#P 6140);
AMPLEX RED™ (10-Acetyl 3,7 Dihydroxyphenoxazine; e.g., Molecular Probes Cat#A 12222);
Glucose;
Sodium Phosphate Buffer, pH 7.5, 50 mM;
Dimethyl Sulfoxide (DMSO);
Black microtiter plates;
Fluorescent plate reader (e.g., Tecan, SpectraMax, etc.).

The following stock solutions in the pH 7.5 sodium phosphate buffer are needed, unless otherwise specified. All of these stock solutions are stable for several weeks when stored at the recommended temperatures.
Glucose Oxidase: make a 100 U/ml solution, to be kept at 4° C.;
Horse Radish (HR) Peroxidase: make a 40 U/ml solution, to be kept at 4° C.;
AMPLEX RED™ (10-acetyl-3,7-dihydroxyphenoxazine): dissolve 5 mg into 3.880 ml of DMSO to make a 5 mM solution (molecular weight (MW) of AMPLEX RED™ is 257.25). Keep this solution in a dark vial at −20° C.;
Glucose: to prevent microbial growth and consumption of your glucose stock solution, prepare in 10 mM Na-Azide and store frozen.

The working stock of reagent should be made just prior to analysis. Assuming each reaction uses 45 μl of reagent, calculate the volume of reagent you will make from the number of samples you have, e.g. 100 samples is 4.5 ml working stock reagent. Make slightly more reagent than you will need, to avoid sucking air on the last row of samples. The AAO working stock reagent is your sodium phosphate buffer containing 1% (v/v) of each of stock enzyme solutions (1% of 100 U/ml Glucose Oxidase and 1% of 20 U/ml HR Peroxidase) and 1% of fluorescent reagent (5 mM 10-acetyl-3,7-dihydroxyphenoxazine, or AMPLEX RED™).

Pipette 5 ul from your enzymatic reaction plate into a black microtiter plate (e.g., either a 96- or 384- well plate), and add 45 ul of working stock reagent. Be sure to include a standard curve of glucose in a plate so that you can compare plates incubated for slightly different times. Incubate in the dark for approximately 15-20 minutes and take an endpoint reading on a fluorescent plate reader. Recommended excitation and emission spectra for resorufin (the product of Amplex Red) are 545 nm ex/590 nm em. Be careful not to let your assay pH fall below 6.5, as fluorescence will decrease around the pKa of resorufin (approximate 6.0).

Exemplary Characterization Assay: PASC Assay for CBH Activity Screening

This exemplary assay can be used to determine if an enzyme has cellobiohydrolase activity and is within the scope of the claimed invention. In one embodiment, the objective of the assay is to determine if cellobiohydrolase subclones are active using PASC (Phosphoric Acid Swollen Cellulose) as substrate.

Exemplary Assay Conditions:
100 ul reaction volume;
50 mM pH5 buffer (sodium acetate) or 50 mM pH7 buffer (sodium phosphate);
0.75% PASC (neutral pH);
20 ul enzyme prep soluble fraction (1:5 dilution in reaction);
(+) control and (−) control included;
Reaction in 96 well PCR plate with foil seal;
Overnight reaction at 37° C. using thermocycler (or heat block).

Exemplary Analysis:
Glucose Oxidase analysis (fluorescence measured at 545/590 using SPECTRAMAX™ reader)

Materials:
96 well PCR plate (used for the reaction);
Black 384 well plate with black bottom (used for glucose oxidase detection);
Foil seal;
1.5% PASC stock solution;
CBH subclones samples (lysed and spun down);
MEGAZYME™ (Wicklow, Ireland) CBHI sample (positive control);
Vector with no insert sample (negative control);
500 mM pH5 sodium acetate stock solution;
500 mM pH7 sodium phosphate stock solution;
Thermocycler or heat block (at 37° C. for reaction);
SPECTRAMAX™ fluorescence reader (Molecular Devices Corporation, Sunnyvale, Calif.) for glucose oxidase detection.
Glucose Oxidase kit:
   1) 800 mM pH7.4 phosphate buffer stock
   2) Purified β-glucosidase (for example, the exemplary SEQ ID NO:424 enzyme of the invention, encoded, e.g., by SEQ ID NO:423)
   3) 2500/250 U/ml Glucose Oxidase/Horse Radish (HR) Peroxidase cocktail
   4) 50 mM Amplex red stock (light sensitive)

Exemplary Protocol for PASC Reaction:
1) Add 20 ul (−) control (vector no insert) to well A1 and C1 in 96 well PCR plate. A1 will be the negative control for reactions at pH5. C1 will be the negative control for reactions at pH7.
2) Dilute 1:20 CBHI stock (Megazyme) (20 ul stock+380 ul water)
3) Add 20 ul diluted CBHI to wells A2 and C2. A2 will be the (+) control for reactions at pH5. C2 will be the (+) control for reactions at pH7.
4) Add 20 ul CBH subclone samples to rows A and C (if more than 10 samples, continue adding to row B and D). RowA will be reactions at pH 5. Row C will be reactions at pH7.
5) Add 20 ul autoclaved water to wells in row A and C containing samples.
6) Add 10 ul 500 mM pH5 buffer stock to row A (50 mM in reaction)
7) Add 10 ul 500 mM pH7 buffer stock to Row C (50 mM in reaction)
8) Using scissors, clip the tips of 200 ul Rainin pipet tips. Pipet tips must be modified to draw up PASC stock solution.
9) Add 50 ul 1.5% PASC stock to all wells containing samples. Mix well using pipet.
10) Seal PCR plate well with foil seal and place plate in thermocycler (or heat block).
11) Let PCR plate incubate at 37° C. overnight.

Exemplary Protocol for Glucose Oxidase Analysis:
1) Remove PCR plate from 37° C. incubator and spin down plate for 5 minutes at 4,000 RPM (using Eppendorf centrifuge).
2) Using a multi-channel pipet, transfer 25 ul reaction supernatant to a black 384 well detection plate. Make sure not to transfer any of the pellet to detection plate (supernatant only).
3) Make 2× glucose oxidase cocktail (volume depends on number of samples):

| Component | [Stock] | | 2x Cocktail | |
|---|---|---|---|---|
| Sterile dI water | n/a | | qs | |
| Sodium phosphate pH7.4 | 800 | mM | 100 | mM |
| B-Glc (SEQ ID NO: 424) | variable | | 0.01 | U/ml |
| GO/HRP mix | 2500/250 | U/ml | 10/1 | U/ml |
| Amplex red | 50 | mM | 0.1 | mM |

Glucose Oxidase (GO) is from Sigma (#G7141-50KU). Dissolve all 50,000 units in 5 ml 50 mM phosphate pH7.4 buffer.
Horse Radish Peroxidase (HRP) is from Sigma (#P2088-5KU). Dissolve all 5,000 units in 5 ml 50 mM phosphate pH7.4 buffer.
GO and HRP are then combined in equal volumes (2,500/250 GO/HRP).
Amplex Red is from Molecular Probe (#A22177). Dissolve 10mg vial in 0.777 ml DMSO to obtain 50 mM stock. Store at −20C protected from light.
4) Add 25 ul of 2× glucose oxidase cocktail to wells in 384 well detection plate containing reaction supernatant. Mix well and avoid formation of bubbles.
5) Let detection plate incubate at room temperature for 30 minutes. Protect plate from light during incubation.
6) Read plate on a SPECTRAMAX™ fluorescence plate reader at 545/590 nm.
7) Save data as a text file and store data. Open up data in EXCEL™ sheet and analyze results.
8) Data analysis:
   CBH subclones deemed “active” must have much higher 545/590 values than those of the negative control. For example, a sample with a value of 1200 would not be considered active if the negative control is 1000. Conversely, a sample with a value of 2500 would be considered active if the negative control is 1000.
   Samples performing with similar or higher values than the positive control to are deemed “highly active.”
   All active CBH subclones will be further characterized on more relevant substrates (AVICEL® Microcrystalline Cellulose (MCC) (FMC Corporation, Philadelphia, Pa.), or a biomass target, e.g., a bagasse, corn fiber, etc.).
   Interpretation of glucose oxidase data is relatively subjective, thus it is very important to have a reliable positive and negative control each time an experiment is performed.

Exemplary CBH Characterization Assay

This exemplary assay can be used to determine if an enzyme has cellulase or cellobiohydrolase (CBH) activity and is within the scope of the claimed invention. This exemplary activity-based screen is for identifying and screening for cellobiohydrolases and other cellulases. Lambda libraries are screened in 384-well plates for activity on microcrystalline cellulose (AVICEL®) and cellulase activity is detected in an enzyme-coupled reaction that includes a β-glucosidase and Invitrogen's glucose oxidase glucose detection assay.

Primary Screen

Preparation
1. Prepare and titer a sufficient amount of E. coli host in MgSO₄ at OD1.
2. Titer the amplified lambda library.
3. Label plates with bar-codes for the robot.

4. Schedule the robot run.
5. Make sure there are sufficient amounts of plates, top agar, reagents, autoclaved reagent bottles, etc.

Calculations
1. How much screening culture will you need?
   Example: if 145 plates will be screened at 25 μL per well, with a safety cushion of 10 extra plates-worth, you will need about 1.5 liters of screening culture.
2. How much E. coli host prep will you need? The culture should be at an initial OD₆₀₀ of about 0.03.
   Example: for 1.5 liters of culture, you will need 45 mL of OD 1 host prep.
3. How much library will you need?
   Example: for an initial seed density of 2 clones per well, you will need a starting concentration of 0.08 phage per μL (i.e., 2 phage in 25 μL). For 1.5 liters of culture, you will need 1.2×10⁵ phage clones. If the titer of the library is 1.5×10⁶ per μL for example, you will need to add 8 μL of a 1/100 dilution for this screen. Use SM buffer for making dilutions of lambda libraries.
4. How much NZY and AVICEL® will you need? The AVICEL® concentration in the screening culture should be around 5% in 1× NZY medium.
   Example: for 1.5 liters of culture, you will need 577 mL of 13% dispersed AVICEL® stock. You will also need 750 mL of 2× NZY to result in a final concentration of 1× NZY. You will also need to qs the culture to 1.5 liters with sterile water (128 mL in the case of this example).

Day 1
1. Combine the calculated amounts of E. coli host and lambda library in a suitable sterile container. Mix gently and allow phage adsorption to occur at room temperature for 15 minutes.
2. Meanwhile, combine the calculated volumes of 2× NZY medium, 13% dispersed AVICEL® stock and sterile water in a suitable sterile container. The AVICEL® will flocculate in the presence of NZY, giving it a “curdled milk” appearance. Mix the suspension on high with a stir bar as thoroughly as possible to avoid large clumps.
3. Combine the cell/phage suspension with the NZY/AVICEL® screening medium and gently mix. This is the screening culture.
4. Concurrent Titer: in a sterile 2059 tube, combine 250 μL of OD1 E. coli prep with 500 μL of screening culture, add 7 mL of molten top agar, plate on a 150 mm NZY plate, and incubate O/N at 37° C. For a seed density of 2 per well, this should result in about 40 plaques.
   a. Next Day: Count plaques on plate and update report on clones screened using: (2×(# plaques))*(9.6 ml×(# of plates screened))
5. Using a sterile TITERTEK™ (Huntsville, Ala.) head, load the remainder of the screening culture into bar-coded 384-well plates at 25 μL per well. Perform this in a laminar flow hood if possible.
6. Control Plates: prepare at least two 384-well plates with assay controls per library screened.
   a. Prepare culture medium with at the following final concentrations: 5% dispersed AVICEL®; 1× NZY; E. coli host at OD₆₀₀ 0.03
   b. To one batch, add positive control phage “D7” to culture medium at a concentration of around 0.2-0.4 phage per μL; to another batch, add negative control phage “N38” (OGL7) at the same concentration
   c. Dispense into black 384-well plates at 25 μL per well, positive control in columns 1-12, negative control in columns 13-24.
7. Incubate 384-well plates at 37° C. overnight in a humidified incubator. Take any necessary precautions to minimize evaporation/condensation problems from well-to-well and from plate-to-plate.

Day 2
1. Prepare a robotic script for the number of plates being screened:
   a. 30 minute room temperature incubation between cocktail addition and plate read.
   b. Use 560/610 filter set for the first read of each plate.
   c. Use UV/blue filter set for the second (reference) read of each plate.
2. Prepare 2× assay cocktail (cap tightly and shield from light); you will generally need about the same volume as the amount needed for screening culture on Day 1. Wait to add DMSO to Amplex Red, and Amplex Red to cocktail mixture until placing on the robot. This will decrease oxidation of Amplex Red. Add the following components in order:

| | | | | | Example | |
|---|---|---|---|---|---|---|
| Component | 2x Cocktail | | [Stock] | | (1.5 L) | |
| Sterile dI water | n/a | | n/a | | qs | |
| Na Phosphate Buffer, | 100 | mM | 800 | mM | 188 | mL |
| pH 7.4 | | | | | | |
| SEQ ID NO: 424 | 0.01 | U/mL | Variable | | Calc | |
| GO/HRP mix | 10/1 | U/mL | 2500/250 | U/mL | 6 | mL |
| Amplex Red | 0.1 | mM | 50 | mM | 3 | mL |

3. Bring incubated plates, assay cocktail and sterile TITERTEK™ head to Robot in Engineering.
4. Stack plates with barcode facing out on carousel in incubator 1 starting with column one and filling down the column. Note: place control plates in the first positions and after the final plate of each library.
5. Attach TITERTEK™ head to TITERTEK™ 1
6. Place assay bottle in tilted position using clamp device, cover in foil, place argon or nitrogen gas nozzle into bottle.
7. Prime the tubing.
8. Log on to computer and start the run.
   a. Check Filter emissions/excitations for 560/610 nm and UV/blue filters

Day 3
1. For each plate, normalize the red readings by dividing them by the blue readings to reduce fluid-based artifacts.
2. Generate a hit list for the cherrypicker.

Exemplary Secondary Screen—Automated Method

Day 1
1. Cherry pick designated hits into 200 ul/well SM buffer in a CP_Master 96 well costar plate.
2. Save 1° screening plates at 4° C. until breakout is complete.

Day 2
1. Plate 0.5 μL of each 1° hit from CP_Master plate with E. coli host onto 90 mm NZY plates.
2. Plate “D7” positive and “N38” negative controls (aim for 50-100 plaques per plate) on separate 90 mm NZY plates.
3. Incubate plates overnight in a dry 37° C. incubator.

Day 3
1. Fill 384 well Sec_Master plates with 25 μL/well of 5% dispersed AVICEL® in 1× NZY+OD 0.03 E. coli host.
2. Bring plates to Colony picker and “colony pick” 16 plaques per primary hit plate to a column of a 384 well plate (Columns 1-20)
3. Pick “D7” positive control into column 21 and “N38” negative control into column 22.
4. Incubate in humidified incubator set to 37° C. overnight.

Day 4
1. Prepare 2× assay cocktail as described for the primary screen.
2. Run assay on Robot as described above.
3. Cherry Pick 2° hits into 96 well Sec_PCR plate.
4. Store phage stocks at 4° C.

Exemplary Secondary Screen—Manual Method

Day 1
1. If primary screening plates have not been cherry-picked, take 1 μL of each hit from the primary screening plate and dilute it in 500μL SM buffer. Plate 1 μL of this dilution with E. coli host onto NZY agar plates as described.
2. If using cherry-picked hits, plate 0.5 μL of each hit from the CP_Master plate as described above.
3. Plate “D7” positive and “N38” negative controls (aim for 50-100 plaques per plate) on separate 90 mm NZY plates.
4. Incubate plates overnight in a dry 37° C. incubator.

Day 2
1. Prepare 384-well recipient plates for picking. Dispense 25 μL/well of the following suspension: 5% dispersed AVICEL® in 1× NZY with E. coli host at OD₆₀₀ 0.03.
2. Use sterile toothpicks or sterile pipet tips to gently touch the surface of each isolated plaque and transfer to a well of the 384-well recipient plate. Pick 16 isolates (one column-worth) per 1° hit.
3. Incubate the plate(s) (“breakout plate”) in humidified incubator set to 37° C. overnight.

Day 3
1. Prepare 2× assay cocktail as described for the primary screen.
2. Add 2× assay cocktail to “breakout plate” at 25 μL/well, incubate at room temperature (shielded from light) for 30 minutes, then read fluorescence at both 535/595 nm and 360/465 nm.
3. Divide the red readings by the blue and identify any 2° hits.
4. If a 2° hit is identified, pull the contents of the well corresponding to a positive isolate and add to 1 mL SM buffer+100 μL CHCl₃. Vortex and store phage stock at 4° C.

### Reagents and Supplies:

### 2× NZY Concentrate, 13% Dispersed AVICEL®

Measure 130 grams AVICEL® microcrystalline cellulose (FMC Biopolymer (Philadelphia, Pa.): type PH 105, grade NF/EP) into a clean, high-speed blender and add 18 MΩ dI water to the 1 L measuring line (about 916 mL). Close the lid and blend at highest speed for 20 minutes. Transfer the suspension to an autoclave-safe container and autoclave using the 30 minute “liquid” cycle. Store at room temperature.

### Na Phosphate Buffer pH 7.4 (800 mM Stock)

Phosphate buffer stock is made at 800 mM to avoid precipitation that sometimes occurs with 1 M stocks. For 1 Liter of 800 mM buffer, combine 90.7 grams Na₂HPO₄ (MW 141.96) with 22.2 grams NaH₂PO₄.H₂O (MW 137.99) and dissolve in about 950 mL dI water. Adjust the pH to 7.4 if necessary with either NaOH or phosphoric acid, then either sterile filter or autoclave.

### SEQ ID NO:424 Enzyme—Control; GO/HRP Mix

Glucose oxidase: Sigma #G7141-50KU. Dissolve all 50,000 units in 5 mL 50 mM phosphate, pH 7.4 buffer.

Horseradish peroxidase: Sigma #P2088-5KU. Dissolve all 5,000 units in 5 mL 50 mM phosphate, pH 7.4 buffer.

Combine Glucose oxidase and HRP solutions. Use a sterile syringe to add an equal volume (10 mL) of sterile glycerol. Mix well. This gives 20 mL of solution with final concentrations: 2,500 U/mL GO; 250 U/mL HRP.

### Amplex Red

Molecular Probes #A22177 (10×10-mg vials, MW =257.25). Dissolve each 10 mg vial in 0.777 mL DMSO to produce a 50 mM stock. Pool as needed. Store unused stocks at −20° C. protected from light.

### Black 384-Well Plates

Costar 384-well plates 3709 Fisher 07-200-652

### Preparation of E. Coli Host Cultures

### Components Needed

O/N culture or streak plate of the E. coli host. Use LB medium+20 μg/mL tetracycline antibiotic for liquid cultures and streak plates.
sterile 250 mL shake flask
LB supplemented with 20 μg/mL tetracycline
sterile centrifuge tubes
sterile 10 mM MgSO4 solution

### Protocol

1. In a sterile 250-mL flask, inoculate 100 mL LB medium+20 μg/mL tetracycline with 1 mL of overnight E. coli host culture.
2. Grow the culture in a 37° C./240 rpm shaker to OD₆₀₀ 0.8-1.0 (typically 2-4 hours).
3. Centrifuge the culture at 1,100× g for 10 minutes (e.g., 2351 rpm in Eppendorf 5810R).
4. Gently resuspend the pellet in 10 mM MgSO₄ to OD₆₀₀=1.0.
5. Store prepared host cells on ice or at 4° C. Host preps should be good for about a week.

### General Protocol for Plating Lambda Phage

### Components Needed

OD1 host prep
sterile Falcon 2054 tubes (or 2059 tubes if using 150 mm plates)
molten NZY top agar, equilibrated at 50° C.
NZY plates, warmed to room temperature

The amount of OD1 host prep and molten top agar required depends on the size of the NZY plates used. General guidelines follow:

| Size of NZY plate | Volume OD1 host prep | Volume of molten top agar |
|---|---|---|
| 90 mm | 100 μL | 2.5 mL |
| 150 mm | 250 μL | 7 mL |

### General Protocol

1. Aliquot the recommended amount of OD1 host prep (see table above) into a sterile 2054 or 2059 tube.
2. Carefully pipet the required volume of lambda phage stock into the aliquoted host cells and mix by gentle vortexing.
3. Let the phage adsorb to host by incubating at room temperature for 15 minutes (no shaking required).
4. Plate the phage by adding the appropriate volume of 50° C. molten top agar (see table above) to the tube and quickly pour over NZY plates. Carefully tilt the plate from side to side to ensure a smooth, even distribution before the top agar hardens.
5. Invert plates and incubate overnight in a dry 37° C. incubator. Note that some NZY plates are very moist. To prevent moisture problems during incubation, these plates need to be vented before placing them in the incubator.

### Titering Lambda Libraries

1. Make 10⁻⁵, 10⁻⁶, and 10⁻⁷ dilutions of the library in SM buffer. Note that more conservative dilutions will be necessary for older libraries that have titers less than 10⁵ per μL (<10⁸ per mL).
2. Pipet 100 μL of OD1 host prep to each of three Falcon 2054 tubes.
3. Carefully add 100 μL of each dilution to a separate 2054 tube containing host cells.
4. Adsorb, plate and incubate as described in steps 3-5 of General Protocol For Plating Lambda Phage above.
5. Count plaques and calculate titer of the library stock (in pfu per μL). If possible, use the dilution that results in between 50-200 plaques. For example, if the plate containing 100 μL of 10⁻⁶ dilution gave 157 plaques, the library titer is about 1.6×10⁶ pfu/μL.

The following table summarizes data from these exemplary protocols; to aid in reading the table, for the column labeled “Exemplary Enzyme” (of the invention), for example, the first row reads “7, 8”, which is read as the enzyme having the amino acid sequence of SEQ ID NO:8, encoded, e.g., by the nucleic acid sequence of SEQ ID NO:7; etc. “Avicel” is AVICEL® as described above. GH family means “glycosyl hydrolase” family. “True” and “False” represent “enzymatically active” or “not active”, respectively, under the indicated conditions. Reaction time is in minutes.

| | | | | | Substrate | | | | | | | | |
|---|---|---|---|---|---|---|---|---|---|---|---|---|---|
| Exemplary | GH | Expression | % | | concen- | Enzyme | Reaction | pH 5, | pH 7, | pH 9, | pH 5, | pH 7, | pH 9, |
| enzyme | family | Host | purity | Substrate | tration | loading | Time | 37° C. | 37° C. | 37° C. | 55° C. | 55° C. | 55° C. |
| 7, 8 | 5 | E. coli | | Avicel | 0.004 | 0.1 mg/ml | 48 | TRUE | TRUE | TRUE | TRUE | TRUE | TRUE |
| 427, 428 | 9 | E. coli | ? | Avicel | 0.004 | 0.1 mg/ml | 48 | TRUE | TRUE | TRUE | TRUE | TRUE | FALSE |
| 427, 428 | 9 | E. coli | ? | Avicel | 0.004 | 0.1 mg/ml | 48 | TRUE | TRUE | TRUE | TRUE | TRUE | FALSE |
| 9, 10 | 5 | E. coli | | Avicel | 0.004 | 0.1 mg/ml | 48 | TRUE | TRUE | TRUE | TRUE | TRUE | TRUE |
| 361, 362 | 9 | E. coli | 12.1% | Avicel | 0.005 | 0.1 mg/ml | 44.5 | TRUE | TRUE | TRUE | TRUE | TRUE | TRUE |
| 5, 6 | 6 | E. coli | 3.8% | Avicel | 0.005 | 0.1 mg/ml | 44.5 | TRUE | TRUE | TRUE | TRUE | TRUE | TRUE |
| 13, 14 | 6 | E. coli | 3.1% | Avicel | 0.005 | 0.1 mg/ml | 44.5 | TRUE | TRUE | TRUE | TRUE | FALSE | FALSE |
| 11, 12 | 6 | E. coli | 4.9% | Avicel | 0.005 | 0.1 mg/ml | 44.5 | TRUE | TRUE | TRUE | TRUE | TRUE | FALSE |
| 25, 26 | 6 | P. pastoris | 36.1% | Avicel | 0.004 | 0.1 mg/ml | 46 | TRUE | TRUE | TRUE | TRUE | TRUE | TRUE |
| 37, 38 | 48 | P. pastoris | 2.6% | Avicel | 0.004 | 0.1 mg/ml | 46 | TRUE | TRUE | TRUE | TRUE | TRUE | TRUE |
| 1, 2 | 6 | E. coli | 9.4% | Avicel | 0.005 | 0.1 mg/ml | 44.5 | TRUE | TRUE | TRUE | TRUE | TRUE | TRUE |
| 3, 4 | 6 | P. pastoris | 3.3% | Avicel | 0.004 | 0.1 mg/ml | 48 | TRUE | TRUE | TRUE | TRUE | TRUE | TRUE |
| 23, 24 | 6 | P. pastoris | 8.2% | Avicel | 0.005 | 0.1 mg/ml | 44.5 | FALSE | FALSE | FALSE | TRUE | TRUE | TRUE |
| 353, 354 | 6 | P. pastoris | 15.7% | Avicel | 0.005 | 0.1 mg/ml | 44.5 | FALSE | FALSE | TRUE | TRUE | TRUE | TRUE |
| 39, 40 | 48 | P. pastoris | 5.5% | Avicel | 0.004 | 0.1 mg/ml | 46 | TRUE | TRUE | TRUE | TRUE | TRUE | TRUE |
| 47, 48 | 9 | E. coli | | Avicel | 0.005 | 0.1 mg/ml | 44.5 | TRUE | TRUE | TRUE | FALSE | FALSE | FALSE |
| 49, 50 | 5 | E. coli | 4.8% | Avicel | 0.005 | 0.1 mg/ml | 44.5 | TRUE | TRUE | TRUE | FALSE | FALSE | FALSE |
| 51, 52 | 9 | E. coli | 3.9% | Avicel | 0.005 | 0.1 mg/ml | 44.5 | FALSE | TRUE | FALSE | FALSE | FALSE | FALSE |
| 43, 44 | 9 | E. coli | 6.4% | Avicel | 0.005 | 0.1 mg/ml | 44.5 | FALSE | TRUE | TRUE | FALSE | FALSE | FALSE |
| 57, 58 | 9 | E. coli | 8.3% | Avicel | 0.005 | 0.1 mg/ml | 44.5 | TRUE | TRUE | TRUE | FALSE | FALSE | FALSE |
| 55, 56 | 5 | E. coli | 12.4% | Avicel | 0.005 | 0.1 mg/ml | 44.5 | TRUE | TRUE | TRUE | FALSE | FALSE | FALSE |
| 59, 60 | 45 | E. coli | 5.8% | Avicel | 0.005 | 0.1 mg/ml | 44.5 | TRUE | TRUE | TRUE | FALSE | FALSE | FALSE |
| 61, 62 | 9 | E. coli | 9.8% | Avicel | 0.005 | 0.1 mg/ml | 44.5 | FALSE | TRUE | TRUE | FALSE | FALSE | FALSE |
| 53, 54 | 5 | E. coli | 11.4% | Avicel | 0.005 | 0.1 mg/ml | 44.5 | FALSE | TRUE | TRUE | FALSE | FALSE | FALSE |
| 65, 66 | 5 | E. coli | 10.7% | Avicel | 0.005 | 0.1 mg/ml | 44.5 | TRUE | TRUE | TRUE | FALSE | FALSE | FALSE |
| 41, 42 | 5 | E. coli | 3.0% | Avicel | 0.004 | 0.1 mg/ml | 47 | TRUE | TRUE | TRUE | TRUE | TRUE | FALSE |
| 365, 366 | 8 | E. coli | 8.6% | Avicel | 0.005 | 0.1 mg/ml | 44.5 | TRUE | TRUE | TRUE | TRUE | TRUE | TRUE |
| 67, 68 | 5 | E. coli | 6.4% | Avicel | 0.004 | 0.1 mg/ml | 47 | TRUE | TRUE | TRUE | FALSE | FALSE | FALSE |
| 17, 18 | 16 | E. coli | ? | Avicel | 0.004 | 0.1 mg/ml | 47 | TRUE | TRUE | TRUE | FALSE | FALSE | FALSE |
| 77, 78 | 5 | E. coli | 4.4% | Avicel | 0.005 | 0.1 mg/ml | 44.5 | TRUE | TRUE | TRUE | FALSE | FALSE | FALSE |
| 73, 74 | 5 | E. coli | 7.4% | Avicel | 0.005 | 0.1 mg/ml | 44.5 | TRUE | TRUE | TRUE | FALSE | FALSE | FALSE |
| 363, 364 | 18 | E. coli | 11.5% | Avicel | 0.005 | 0.1 mg/ml | 44.5 | TRUE | TRUE | TRUE | TRUE | TRUE | TRUE |
| 45, 46 | ARF | E. coli | ? | Avicel | 0.004 | 0.1 mg/ml | 47 | TRUE | TRUE | TRUE | TRUE | TRUE | TRUE |
| 63, 64 | 9 | E. coli | 8.0% | Avicel | 0.004 | 0.1 mg/ml | 47 | TRUE | FALSE | FALSE | FALSE | FALSE | FALSE |
| 75, 76 | 9 | E. coli | 10.5% | Avicel | 0.004 | 0.1 mg/ml | 47 | FALSE | TRUE | TRUE | FALSE | TRUE | TRUE |
| 87, 88 | 5 | E. coli | 10.7% | Avicel | 0.005 | 0.1 mg/ml | 44.5 | TRUE | TRUE | TRUE | FALSE | FALSE | FALSE |
| 83, 84 | 5 | E. coli | 10.4% | Avicel | 0.005 | 0.1 mg/ml | 44.5 | FALSE | TRUE | TRUE | FALSE | FALSE | FALSE |
| 81, 82 | ARF | E. coli | ? | Avicel | 0.005 | 0.1 mg/ml | 44.5 | TRUE | TRUE | TRUE | FALSE | FALSE | FALSE |
| 89, 90 | 5 | E. coli | 5.0% | Avicel | 0.005 | 0.1 mg/ml | 44.5 | TRUE | TRUE | TRUE | TRUE | TRUE | TRUE |
| 85, 86 | 9 | E. coli | 8.7% | Avicel | 0.005 | 0.1 mg/ml | 44.5 | TRUE | TRUE | TRUE | TRUE | TRUE | TRUE |
| 79, 80 | 5 | E. coli | 14.9% | Avicel | 0.005 | 0.1 mg/ml | 44.5 | TRUE | TRUE | TRUE | FALSE | FALSE | FALSE |
| 35, 36 | 6 | A. niger | >60% | Avicel | 0.004 | 0.1 mg/ml | 46.5 | TRUE | TRUE | TRUE | TRUE | TRUE | FALSE |
| 71, 72 | 45 | E. coli | 4.2% | Avicel | 0.005 | 0.1 mg/ml | 44.5 | FALSE | FALSE | FALSE | FALSE | FALSE | FALSE |
| 91, 92 | 48 | E. coli | 3.2% | Avicel | 0.004 | 0.1 mg/ml | 46 | TRUE | FALSE | FALSE | TRUE | FALSE | FALSE |
| 93, 94 | 48 | E. coli | 3.8% | Avicel | 0.005 | 0.1 mg/ml | 44.5 | FALSE | TRUE | FALSE | FALSE | FALSE | FALSE |
| 95, 96 | 48 | E. coli | 6.2% | Avicel | 0.005 | 0.1 mg/ml | 44.5 | FALSE | FALSE | FALSE | TRUE | FALSE | FALSE |
| 99, 100 | 48 | E. coli | 4.4% | Avicel | 0.005 | 0.1 mg/ml | 44.5 | FALSE | FALSE | FALSE | FALSE | FALSE | FALSE |
| 131, 132 | 5 | E. coli | 10.7% | Avicel | 0.004 | 0.1 mg/ml | 48 | FALSE | TRUE | TRUE | FALSE | TRUE | TRUE |
| 133, 134 | 5 | E. coli | 4.1% | Avicel | 0.004 | 0.1 mg/ml | 48 | TRUE | TRUE | TRUE | FALSE | FALSE | FALSE |
| 109, 110 | 5 | E. coli | 2.7% | Avicel | 0.004 | 0.1 mg/ml | 46 | TRUE | TRUE | TRUE | FALSE | FALSE | FALSE |
| 117, 118 | 5 | E. coli | 5.5% | Avicel | 0.004 | 0.1 mg/ml | 48 | TRUE | TRUE | TRUE | TRUE | TRUE | TRUE |
| 355, 356 | 7 | A. niger | 70.0% | Avicel | 0.005 | 0.1 mg/ml | 44.5 | TRUE | TRUE | FALSE | TRUE | TRUE | FALSE |
| 33, 34 | 7 | A. niger | 70.0% | Avicel | 0.005 | 0.1 mg/ml | 44.5 | TRUE | TRUE | FALSE | TRUE | TRUE | FALSE |
| 359, 360 | 7 | A. niger | 80.0% | Avicel | 0.005 | 0.1 mg/ml | 44.5 | TRUE | TRUE | FALSE | TRUE | TRUE | FALSE |
| 121, 122 | 5 | E. coli | ? | Avicel | 0.004 | 0.1 mg/ml | 47 | FALSE | TRUE | TRUE | TRUE | TRUE | TRUE |
| 139, 140 | 48 | E. coli | 4.4% | Avicel | 0.004 | 0.1 mg/ml | 46.5 | TRUE | TRUE | TRUE | TRUE | TRUE | TRUE |
| 143, 144 | 5 | E. coli | ? | Avicel | 0.004 | 0.1 mg/ml | 47 | TRUE | TRUE | TRUE | TRUE | TRUE | TRUE |
| 145, 146 | 9 | E. coli | 26.1% | Avicel | 0.004 | 0.1 mg/ml | 47 | FALSE | TRUE | TRUE | FALSE | TRUE | TRUE |
| 147, 148 | 5 | E. coli | 17.3% | Avicel | 0.004 | 0.1 mg/ml | 47 | TRUE | TRUE | TRUE | FALSE | FALSE | FALSE |
| 151, 152 | 9 | E. coli | 21.7% | Avicel | 0.004 | 0.1 mg/ml | 47 | FALSE | TRUE | FALSE | FALSE | FALSE | FALSE |
| 153, 154 | 5 | E. coli | v. low | Avicel | 0.004 | 0.1 mg/ml | 47 | TRUE | TRUE | TRUE | FALSE | FALSE | FALSE |
| 157, 158 | 5 | E. coli | 3.9% | Avicel | 0.004 | 0.1 mg/ml | 47 | TRUE | TRUE | TRUE | FALSE | FALSE | FALSE |
| 159, 160 | 5 | E. coli | 10.9% | Avicel | 0.004 | 0.1 mg/ml | 47 | TRUE | TRUE | TRUE | FALSE | FALSE | TRUE |
| 167, 168 | 5 | E. coli | 8.4% | Avicel | 0.004 | 0.1 mg/ml | 47 | TRUE | TRUE | TRUE | FALSE | FALSE | FALSE |
| 141, 142 | 5 | E. coli | 3% | Avicel | 0.004 | 0.1 mg/ml | 46.5 | FALSE | FALSE | FALSE | FALSE | FALSE | FALSE |
| 161, 162 | 45 | E. coli | 3.1% | Avicel | 0.004 | 0.1 mg/ml | 47 | FALSE | TRUE | TRUE | FALSE | FALSE | FALSE |
| 105, 106 | 5 | E. coli | ? | Avicel | 0.004 | 0.1 mg/ml | 47 | FALSE | FALSE | TRUE | FALSE | FALSE | FALSE |
| 129, 130 | 5 | E. coli | 15.3% | Avicel | 0.004 | 0.1 mg/ml | 47 | FALSE | TRUE | TRUE | FALSE | TRUE | TRUE |
| 107, 108 | 45 | E. coli | 6.0% | Avicel | 0.004 | 0.1 mg/ml | 47 | TRUE | TRUE | TRUE | TRUE | TRUE | TRUE |
| 103, 104 | 5 | E. coli | ? | Avicel | 0.004 | 0.1 mg/ml | 46.5 | TRUE | TRUE | FALSE | FALSE | FALSE | FALSE |
| 111, 112 | 5 | E. coli | 7.7% | Avicel | 0.004 | 0.1 mg/ml | 47 | TRUE | TRUE | TRUE | TRUE | FALSE | FALSE |
| 169, 170 | 48 | E. coli | ? | Avicel | 0.004 | 0.1 mg/ml | 48 | TRUE | TRUE | TRUE | TRUE | TRUE | TRUE |
| 171, 172 | 48 | E. coli | 7.6% | Avicel | 0.004 | 0.1 mg/ml | 48 | TRUE | TRUE | FALSE | FALSE | FALSE | FALSE |
| 175, 176 | 48 | E. coli | 11.9% | Avicel | 0.004 | 0.1 mg/ml | 48 | TRUE | TRUE | TRUE | TRUE | TRUE | TRUE |
| 177, 178 | 48 | E. coli | ? | Avicel | 0.004 | 0.1 mg/ml | 48 | FALSE | TRUE | TRUE | FALSE | TRUE | TRUE |
| 357, 358 | 6 | A. niger | >85% | Avicel | 0.004 | 0.1 mg/ml | 46.5 | TRUE | TRUE | TRUE | TRUE | TRUE | TRUE |
| 155, 156 | 9 | E. coli | 14.5% | Avicel | 0.004 | 0.1 mg/ml | 46 | FALSE | TRUE | TRUE | FALSE | FALSE | FALSE |
| 173, 174 | 48 | E. coli | 13.3% | Avicel | 0.004 | 0.1 mg/ml | 46 | TRUE | TRUE | TRUE | TRUE | TRUE | FALSE |
| 149, 150 | 5 | E. coli | 4.7% | Avicel | 0.004 | 0.1 mg/ml | 45.5 | FALSE | FALSE | FALSE | FALSE | FALSE | FALSE |
| 183, 184 | 6 | E. coli | ? | Avicel | 0.004 | 0.1 mg/ml | 46 | FALSE | FALSE | FALSE | FALSE | FALSE | FALSE |
| 185, 186 | 6 | E. coli | <3% | Avicel | 0.004 | 0.1 mg/ml | | FALSE | FALSE | FALSE | FALSE | FALSE | FALSE |
| 187, 188 | 48 | E. coli | 13.6% | Avicel | 0.004 | 0.1 mg/ml | 46 | TRUE | TRUE | TRUE | TRUE | TRUE | FALSE |
| 191, 192 | 6 | E. coli | 4.3% | Avicel | 0.004 | 0.1 mg/ml | 46 | FALSE | TRUE | TRUE | FALSE | TRUE | FALSE |
| 113, 114 | 5 | E. coli | 5.5% | Avicel | 0.004 | 0.1 mg/ml | 46 | FALSE | FALSE | FALSE | FALSE | FALSE | FALSE |
| 113, 114 | 5 | E. coli | <3% | Avicel | 0.004 | 0.1 mg/ml | 45.5 | FALSE | FALSE | FALSE | FALSE | FALSE | FALSE |
| 113, 114 | 5 | E. coli | | Avicel | 0.004 | 0.1 mg/ml | 46 | FALSE | FALSE | FALSE | FALSE | FALSE | FALSE |
| 119, 120 | 5 | E. coli | <3% | Avicel | 0.004 | 0.1 mg/ml | 45.5 | FALSE | TRUE | TRUE | FALSE | FALSE | FALSE |
| 31, 32 | 7 | A. niger | | Avicel | 0.004 | 0.1 mg/ml | 46 | TRUE | TRUE | FALSE | TRUE | TRUE | FALSE |
| 369-371 | 7 | A. niger | >90% | Avicel | 0.004 | 0.1 mg/ml | 45.5 | TRUE | TRUE | TRUE | TRUE | TRUE | TRUE |
| 369-371 | 7 | A. niger | | Avicel | 0.004 | 0.1 mg/ml | 46 | TRUE | TRUE | TRUE | TRUE | TRUE | TRUE |
| 189, 190 | 48 | E. coli | <3% | Avicel | 0.004 | 0.1 mg/ml | | FALSE | FALSE | FALSE | FALSE | FALSE | FALSE |
| 179, 180 | 48 | E. coli | <3% | Avicel | 0.004 | 0.1 mg/ml | | FALSE | FALSE | FALSE | FALSE | FALSE | FALSE |
| 163, 164 | 6 | E. coli | <3% | Avicel | 0.004 | 0.1 mg/ml | | FALSE | FALSE | FALSE | FALSE | FALSE | FALSE |
| 181, 182 | 6 | E. coli | <3% | Avicel | 0.004 | 0.1 mg/ml | | FALSE | FALSE | FALSE | FALSE | FALSE | FALSE |
| 165, 166 | 6 | E. coli | 5.0% | Avicel | 0.004 | 0.1 mg/ml | | FALSE | FALSE | FALSE | FALSE | FALSE | FALSE |
| 367, 368 | 9 | E. coli | 11.1% | Avicel | 0.004 | 0.1 mg/ml | 46 | FALSE | TRUE | TRUE | FALSE | TRUE | FALSE |
| 201, 202 | 6 | E. coli | <3% | Avicel | 0.004 | 0.1 mg/ml | 45.5 | FALSE | TRUE | FALSE | FALSE | FALSE | FALSE |
| 135, 136 | 48 | E. coli | <3% | Avicel | 0.004 | 0.1 mg/ml | 45.5 | TRUE | TRUE | TRUE | FALSE | FALSE | FALSE |
| 135, 136 | 48 | E. coli | | Avicel | 0.004 | 0.1 mg/ml | 48 | TRUE | TRUE | TRUE | TRUE | TRUE | TRUE |
| 207, 208 | 48 | E. coli | 2.2% | Avicel | 0.004 | 0.1 mg/ml | 45.5 | FALSE | FALSE | FALSE | FALSE | FALSE | FALSE |
| 209, 210 | 6 | E. coli | <3% | Avicel | 0.004 | 0.1 mg/ml | 45.5 | FALSE | TRUE | TRUE | FALSE | FALSE | FALSE |
| 211, 212 | 9 | E. coli | 10.1% | Avicel | 0.004 | 0.1 mg/ml | 45.5 | FALSE | TRUE | FALSE | FALSE | FALSE | FALSE |
| 211, 212 | 9 | E. coli | | Avicel | 0.004 | 0.1 mg/ml | 46 | FALSE | TRUE | FALSE | FALSE | FALSE | FALSE |
| 125, 126 | 5 | E. coli | <3% | Avicel | 0.004 | 0.1 mg/ml | 45.5 | FALSE | FALSE | FALSE | FALSE | FALSE | FALSE |
| 125, 126 | 5 | E. coli | | Avicel | 0.004 | 0.1 mg/ml | 48 | FALSE | FALSE | FALSE | FALSE | FALSE | FALSE |
| 97, 98 | 48 | P. pastoris | 5.3% | Avicel | 0.004 | 0.1 mg/ml | 47.75 | TRUE | TRUE | TRUE | TRUE | TRUE | TRUE |
| 101, 102 | 48 | P. pastoris | 4.6% | Avicel | 0.004 | 0.1 mg/ml | 47.75 | TRUE | TRUE | TRUE | TRUE | TRUE | TRUE |
| 193, 194 | 48 | E. coli | 6.2% | Avicel | 0.004 | 0.1 mg/ml | 45.5 | TRUE | TRUE | TRUE | FALSE | FALSE | FALSE |
| 193, 194 | 48 | E. coli | | Avicel | 0.004 | 0.1 mg/ml | 46 | TRUE | TRUE | TRUE | TRUE | TRUE | TRUE |
| 241, 242 | 48 | E. coli | <3% | Avicel | 0.004 | 0.1 mg/ml | 47.75 | TRUE | TRUE | TRUE | FALSE | FALSE | FALSE |
| 213, 214 | 5 | E. coli | 5.3% | Avicel | 0.004 | 0.1 mg/ml | 47.75 | TRUE | TRUE | TRUE | FALSE | FALSE | FALSE |
| 231, 232 | 9 | E. coli | 6.4% | Avicel | 0.004 | 0.1 mg/ml | 47.75 | FALSE | TRUE | TRUE | FALSE | FALSE | FALSE |
| 247, 248 | 9 | E. coli | 5.5% | Avicel | 0.004 | 0.1 mg/ml | 47.75 | TRUE | TRUE | TRUE | FALSE | FALSE | FALSE |
| 245, 246 | 9 | E. coli | 6.4% | Avicel | 0.004 | 0.1 mg/ml | 47.75 | FALSE | TRUE | TRUE | FALSE | FALSE | FALSE |
| 249, 250 | 5 | E. coli | 5.0% | Avicel | 0.004 | 0.1 mg/ml | 47.75 | TRUE | TRUE | TRUE | FALSE | FALSE | FALSE |
| 235, 236 | 6 | E. coli | | Avicel | 0.004 | 0.1 mg/ml | 48 | TRUE | TRUE | TRUE | TRUE | TRUE | TRUE |
| 263, 264 | 48 | E. coli | | Avicel | 0.004 | 0.1 mg/ml | 48 | TRUE | TRUE | TRUE | TRUE | TRUE | TRUE |
| 281, 282 | 6 | A. niger | 100 | Avicel | 0.004 | 0.1 mg/ml | 46 | TRUE | TRUE | TRUE | TRUE | TRUE | FALSE |
| 261, 262 | 5 | E. coli | <3% | Avicel | 0.004 | 0.1 mg/ml | 46 | TRUE | TRUE | TRUE | TRUE | TRUE | TRUE |
| 261, 262 | 5 | E. coli | 5.8% | Avicel | 0.004 | 0.1 mg/ml | 46 | TRUE | TRUE | TRUE | TRUE | TRUE | TRUE |
| 233, 234 | 5 | E. coli | | Avicel | 0.004 | 0.1 mg/ml | 48 | FALSE | TRUE | TRUE | TRUE | TRUE | TRUE |
| 219, 220 | 6 | E. coli | | Avicel | 0.004 | 0.1 mg/ml | 48 | FALSE | TRUE | TRUE | TRUE | TRUE | TRUE |
| 239, 240 | 6 | E. coli | | Avicel | 0.004 | 0.1 mg/ml | 48 | TRUE | TRUE | TRUE | TRUE | TRUE | TRUE |
| 217, 218 | 6 | E. coli | | Avicel | 0.004 | 0.1 mg/ml | 48 | TRUE | TRUE | TRUE | TRUE | TRUE | TRUE |
| 251, 252 | 45 | E. coli | <3% | Avicel | 0.004 | 0.1 mg/ml | 46 | TRUE | TRUE | TRUE | TRUE | TRUE | FALSE |
| 181, 182 | 6 | S. diversa | 4.3% | Avicel | 0.004 | 0.1 mg/ml | 44.5 | TRUE | TRUE | TRUE | TRUE | TRUE | TRUE |
| 181, 182 | 6 | S. diversa | 2.4% | Avicel | 0.004 | 0.1 mg/ml | 44.5 | TRUE | TRUE | TRUE | TRUE | TRUE | TRUE |
| 225, 226 | 6 | S. diversa | 4.4% | Avicel | 0.004 | 0.1 mg/ml | 44.5 | TRUE | TRUE | TRUE | TRUE | TRUE | TRUE |
| 229, 230 | 6 | S. diversa | 6.3% | Avicel | 0.004 | 0.1 mg/ml | 44.5 | TRUE | TRUE | TRUE | TRUE | TRUE | TRUE |
| 221, 222 | 6 | S. diversa | 3.3% | Avicel | 0.004 | 0.1 mg/ml | 44.5 | TRUE | TRUE | TRUE | TRUE | TRUE | TRUE |
| 223, 224 | 6 | S. diversa | 3.5% | Avicel | 0.004 | 0.1 mg/ml | 44.5 | TRUE | TRUE | TRUE | TRUE | TRUE | TRUE |
| 185, 186 | 6 | S. diversa | 7.9% | Avicel | 0.004 | 0.1 mg/ml | 44.5 | TRUE | TRUE | TRUE | TRUE | TRUE | TRUE |
| 139, 140 | 6 | S. diversa | 3.5% | Avicel | 0.004 | 0.1 mg/ml | 44.5 | TRUE | TRUE | TRUE | TRUE | TRUE | TRUE |
| 165, 166 | 6 | S. diversa | 5.5% | Avicel | 0.004 | 0.1 mg/ml | 44.5 | TRUE | TRUE | TRUE | TRUE | TRUE | TRUE |

| | GH | Expression | % | | Substrate | Enzyme | Reaction | Preferred | Preferred | |
|---|---|---|---|---|---|---|---|---|---|---|
| Exemplary enzyme | family | Host | purity | Substrate | concentration | loading | Time | pH | temperat. | Max conversion |
| 7, 8 | 5 | E. coli | | Avicel | 0.004 | 0.1 mg/ml | 48 | 7 | 55 | 0.62% |
| 427, 428 | 9 | E. coli | ? | Avicel | 0.004 | 0.1 mg/ml | 48 | 5 | 55 | 2.76% |
| 427, 428 | 9 | E. coli | ? | Avicel | 0.004 | 0.1 mg/ml | 48 | 5 | 55 | 4.82% |
| 9, 10 | 5 | E. coli | | Avicel | 0.004 | 0.1 mg/ml | 48 | 7 | 55 | 1.01% |
| 361, 362 | 9 | E. coli | 12.1% | Avicel | 0.005 | 0.1 mg/ml | 44.5 | 5 | 55 | 1.35% |
| 5, 6 | 6 | E. coli | 3.8% | Avicel | 0.005 | 0.1 mg/ml | 44.5 | 5 | 37 | 0.85% |
| 13, 14 | 6 | E. coli | 3.1% | Avicel | 0.005 | 0.1 mg/ml | 44.5 | 5 | 37 | 0.11% |
| 11, 12 | 6 | E. coli | 4.9% | Avicel | 0.005 | 0.1 mg/ml | 44.5 | 5 | 37 | 0.26% |
| 25, 26 | 6 | P. pastoris | 36.1% | Avicel | 0.004 | 0.1 mg/ml | 46 | 9 | 37 | 0.38% |
| 37, 38 | 48 | P. pastoris | 2.6% | Avicel | 0.004 | 0.1 mg/ml | 46 | 7 | 55 | 1.14% |
| 1, 2 | 6 | E. coli | 9.4% | Avicel | 0.005 | 0.1 mg/ml | 44.5 | 5 | 55 | 0.35% |
| 3, 4 | 6 | P. pastoris | 3.3% | Avicel | 0.004 | 0.1 mg/ml | 48 | 5 | 37 | 0.57% |
| 23, 24 | 6 | P. pastoris | 8.2% | Avicel | 0.005 | 0.1 mg/ml | 44.5 | 5 | 55 | 0.19% |
| 353, 354 | 6 | P. pastoris | 15.7% | Avicel | 0.005 | 0.1 mg/ml | 44.5 | 5 | 55 | 0.33% |
| 39, 40 | 48 | P. pastoris | 5.5% | Avicel | 0.004 | 0.1 mg/ml | 46 | 5 | 55 | 1.26% |
| 47, 48 | 9 | E. coli | | Avicel | 0.005 | 0.1 mg/ml | 44.5 | 7 | 37 | 0.19% |
| 49, 50 | 5 | E. coli | 4.8% | Avicel | 0.005 | 0.1 mg/ml | 44.5 | 7 | 37 | 0.34% |
| 51, 52 | 9 | E. coli | 3.9% | Avicel | 0.005 | 0.1 mg/ml | 44.5 | 7 | 37 | 0.30% |
| 43, 44 | 9 | E. coli | 6.4% | Avicel | 0.005 | 0.1 mg/ml | 44.5 | 7 | 37 | 0.18% |
| 57, 58 | 9 | E. coli | 8.3% | Avicel | 0.005 | 0.1 mg/ml | 44.5 | 7 | 37 | 0.25% |
| 55, 56 | 5 | E. coli | 12.4% | Avicel | 0.005 | 0.1 mg/ml | 44.5 | 5 | 37 | 0.40% |
| 59, 60 | 45 | E. coli | 5.8% | Avicel | 0.005 | 0.1 mg/ml | 44.5 | 9 | 37 | 0.08% |
| 61, 62 | 9 | E. coli | 9.8% | Avicel | 0.005 | 0.1 mg/ml | 44.5 | 7 | 37 | 0.20% |
| 53, 54 | 5 | E. coli | 11.4% | Avicel | 0.005 | 0.1 mg/ml | 44.5 | 7 | 37 | 0.47% |
| 65, 66 | 5 | E. coli | 10.7% | Avicel | 0.005 | 0.1 mg/ml | 44.5 | 7 | 37 | 0.52% |
| 41, 42 | 5 | E. coli | 3.0% | Avicel | 0.004 | 0.1 mg/ml | 47 | 5 | 37 | 0.19% |
| 365, 366 | 8 | E. coli | 8.6% | Avicel | 0.005 | 0.1 mg/ml | 44.5 | 7 | 55 | 0.72% |
| 67, 68 | 5 | E. coli | 6.4% | Avicel | 0.004 | 0.1 mg/ml | 47 | 5 | 37 | 0.18% |
| 17, 18 | 16 | E. coli | ? | Avicel | 0.004 | 0.1 mg/ml | 47 | 5 | 37 | 0.17% |
| 77, 78 | 5 | E. coli | 4.4% | Avicel | 0.005 | 0.1 mg/ml | 44.5 | 5 | 37 | 0.46% |
| 73, 74 | 5 | E. coli | 7.4% | Avicel | 0.005 | 0.1 mg/ml | 44.5 | 5 | 37 | 0.37% |
| 363, 364 | 18 | E. coli | 11.5% | Avicel | 0.005 | 0.1 mg/ml | 44.5 | 7 | 55 | 0.40% |
| 45, 46 | ARF | E. coli | ? | Avicel | 0.004 | 0.1 mg/ml | 47 | 5 | 37 | 0.09% |
| 63, 64 | 9 | E. coli | 8.0% | Avicel | 0.004 | 0.1 mg/ml | 47 | 5 | 37 | 0.12% |
| 75, 76 | 9 | E. coli | 10.5% | Avicel | 0.004 | 0.1 mg/ml | 47 | 7 | 37 | 0.06% |
| 87, 88 | 5 | E. coli | 10.7% | Avicel | 0.005 | 0.1 mg/ml | 44.5 | 5 | 37 | 0.22% |
| 83, 84 | 5 | E. coli | 10.4% | Avicel | 0.005 | 0.1 mg/ml | 44.5 | 9 | 37 | 0.25% |
| 81, 82 | ARF | E. coli | ? | Avicel | 0.005 | 0.1 mg/ml | 44.5 | 7 | 37 | 0.17% |
| 89, 90 | 5 | E. coli | 5.0% | Avicel | 0.005 | 0.1 mg/ml | 44.5 | 5 | 37 | 0.57% |
| 85, 86 | 9 | E. coli | 8.7% | Avicel | 0.005 | 0.1 mg/ml | 44.5 | 5 | 55 | 0.23% |
| 79, 80 | 5 | E. coli | 14.9% | Avicel | 0.005 | 0.1 mg/ml | 44.5 | 7 | 37 | 0.42% |
| 35, 36 | 6 | A. niger | >60% | Avicel | 0.004 | 0.1 mg/ml | 46.5 | 5 | 37 | 0.43% |
| 71, 72 | 45 | E. coli | 4.2% | Avicel | 0.005 | 0.1 mg/ml | 44.5 | | | 0.06% |
| 91, 92 | 48 | E. coli | 3.2% | Avicel | 0.004 | 0.1 mg/ml | 46 | 5 | 55 | 0.09% |
| 93, 94 | 48 | E. coli | 3.8% | Avicel | 0.005 | 0.1 mg/ml | 44.5 | 7 | 37 | 0.05% |
| 95, 96 | 48 | E. coli | 6.2% | Avicel | 0.005 | 0.1 mg/ml | 44.5 | 5 | 55 | 0.12% |
| 99, 100 | 48 | E. coli | 4.4% | Avicel | 0.005 | 0.1 mg/ml | 44.5 | | | 0.05% |
| 131, 132 | 5 | E. coli | 10.7% | Avicel | 0.004 | 0.1 mg/ml | 48 | 7 | 37 | 0.48% |
| 133, 134 | 5 | E. coli | 4.1% | Avicel | 0.004 | 0.1 mg/ml | 48 | 9 | 37 | 0.35% |
| 109, 110 | 5 | E. coli | 2.7% | Avicel | 0.004 | 0.1 mg/ml | 46 | 7 | 37 | 0.09% |
| 117, 118 | 5 | E. coli | 5.5% | Avicel | 0.004 | 0.1 mg/ml | 48 | 7 | 37 | 0.21% |
| 355, 356 | 7 | A. niger | 70.0% | Avicel | 0.005 | 0.1 mg/ml | 44.5 | 5 | 37 | 1.94% |
| 33, 34 | 7 | A. niger | 70.0% | Avicel | 0.005 | 0.1 mg/ml | 44.5 | 5 | 55 | 4.56% |
| 359, 360 | 7 | A. niger | 80.0% | Avicel | 0.005 | 0.1 mg/ml | 44.5 | 5 | 55 | 5.12% |
| 121, 122 | 5 | E. coli | ? | Avicel | 0.004 | 0.1 mg/ml | 47 | 9 | 55 | 0.06% |
| 139, 140 | 48 | E. coli | 4.4% | Avicel | 0.004 | 0.1 mg/ml | 46.5 | 5 | 37 | 0.28% |
| 143, 144 | 5 | E. coli | ? | Avicel | 0.004 | 0.1 mg/ml | 47 | 9 | 37 | 0.10% |
| 145, 146 | 9 | E. coli | 26.1% | Avicel | 0.004 | 0.1 mg/ml | 47 | 7 | 37 | 0.09% |
| 147, 148 | 5 | E. coli | 17.3% | Avicel | 0.004 | 0.1 mg/ml | 47 | 5 | 37 | 0.30% |
| 151, 152 | 9 | E. coli | 21.7% | Avicel | 0.004 | 0.1 mg/ml | 47 | 7 | 37 | 0.15% |
| 153, 154 | 5 | E. coli | v. low | Avicel | 0.004 | 0.1 mg/ml | 47 | 5 | 37 | 0.26% |
| 157, 158 | 5 | E. coli | 3.9% | Avicel | 0.004 | 0.1 mg/ml | 47 | 5 | 37 | 0.25% |
| 159, 160 | 5 | E. coli | 10.9% | Avicel | 0.004 | 0.1 mg/ml | 47 | 9 | 37 | 0.36% |
| 167, 168 | 5 | E. coli | 8.4% | Avicel | 0.004 | 0.1 mg/ml | 47 | 5 | 37 | 0.26% |
| 141, 142 | 5 | E. coli | 3% | Avicel | 0.004 | 0.1 mg/ml | 46.5 | | | 0.01% |
| 161, 162 | 45 | E. coli | 3.1% | Avicel | 0.004 | 0.1 mg/ml | 47 | 9 | 37 | 0.07% |
| 105, 106 | 5 | E. coli | ? | Avicel | 0.004 | 0.1 mg/ml | 47 | 9 | 37 | 0.07% |
| 129, 130 | 5 | E. coli | 15.3% | Avicel | 0.004 | 0.1 mg/ml | 47 | 9 | 37 | 0.14% |
| 107, 108 | 45 | E. coli | 6.0% | Avicel | 0.004 | 0.1 mg/ml | 47 | 5 | 37 | 0.12% |
| 103, 104 | 5 | E. coli | ? | Avicel | 0.004 | 0.1 mg/ml | 46.5 | 7 | 37 | 0.07% |
| 111, 112 | 5 | E. coli | 7.7% | Avicel | 0.004 | 0.1 mg/ml | 47 | 9 | 37 | 0.22% |
| 169, 170 | 48 | E. coli | ? | Avicel | 0.004 | 0.1 mg/ml | 48 | 5 | 37 | 0.35% |
| 171, 172 | 48 | E. coli | 7.6% | Avicel | 0.004 | 0.1 mg/ml | 48 | 7 | 37 | 0.32% |
| 175, 176 | 48 | E. coli | 11.9% | Avicel | 0.004 | 0.1 mg/ml | 48 | 5 | 37 | 0.79% |
| 177, 178 | 48 | E. coli | ? | Avicel | 0.004 | 0.1 mg/ml | 48 | 7 | 37 | 0.13% |
| 357, 358 | 6 | A. niger | >85% | Avicel | 0.004 | 0.1 mg/ml | 46.5 | 5 | 55 | 1.98% |
| 155, 156 | 9 | E. coli | 14.5% | Avicel | 0.004 | 0.1 mg/ml | 46 | 7 | 37 | 0.33% |
| 173, 174 | 48 | E. coli | 13.3% | Avicel | 0.004 | 0.1 mg/ml | 46 | 5 | 37 | 0.77% |
| 149, 150 | 5 | E. coli | 4.7% | Avicel | 0.004 | 0.1 mg/ml | 45.5 | | | 0.02% |
| 183, 184 | 6 | E. coli | ? | Avicel | 0.004 | 0.1 mg/ml | 46 | | | 0.01% |
| 185, 186 | 6 | E. coli | <3% | Avicel | 0.004 | 0.1 mg/ml | | | | 0.01% |
| 187, 188 | 48 | E. coli | 13.6% | Avicel | 0.004 | 0.1 mg/ml | 46 | 7 | 37 | 0.48% |
| 191, 192 | 6 | E. coli | 4.3% | Avicel | 0.004 | 0.1 mg/ml | 46 | 9 | 37 | 0.26% |
| 113, 114 | 5 | E. coli | 5.5% | Avicel | 0.004 | 0.1 mg/ml | 46 | | | 0.01% |
| 113, 114 | 5 | E. coli | <3% | Avicel | 0.004 | 0.1 mg/ml | 45.5 | | | 0.02% |
| 113, 114 | 5 | E. coli | | Avicel | 0.004 | 0.1 mg/ml | 46 | | | 0.02% |
| 119, 120 | 5 | E. coli | <3% | Avicel | 0.004 | 0.1 mg/ml | 45.5 | 7 | 37 | 0.04% |
| 31, 32 | 7 | A. niger | | Avicel | 0.004 | 0.1 mg/ml | 46 | 5 | 55 | 2.43% |
| 369-371 | 7 | A. niger | >90% | Avicel | 0.004 | 0.1 mg/ml | 45.5 | 5 | 55 | 1.66% |
| 369-371 | 7 | A. niger | | Avicel | 0.004 | 0.1 mg/ml | 46 | 5 | 37 | 2.27% |
| 189, 190 | 48 | E. coli | <3% | Avicel | 0.004 | 0.1 mg/ml | | | | 0.01% |
| 179, 180 | 48 | E. coli | <3% | Avicel | 0.004 | 0.1 mg/ml | | | | 0.01% |
| 163, 164 | 6 | E. coli | <3% | Avicel | 0.004 | 0.1 mg/ml | | | | 0.01% |
| 181, 182 | 6 | E. coli | <3% | Avicel | 0.004 | 0.1 mg/ml | | | | 0.01% |
| 165, 166 | 6 | E. coli | 5.0% | Avicel | 0.004 | 0.1 mg/ml | | | | 0.02% |
| 367, 368 | 9 | E. coli | 11.1% | Avicel | 0.004 | 0.1 mg/ml | 46 | 7 | 37 | 0.33% |
| 201, 202 | 6 | E. coli | <3% | Avicel | 0.004 | 0.1 mg/ml | 45.5 | 7 | 37 | 0.03% |
| 135, 136 | 48 | E. coli | <3% | Avicel | 0.004 | 0.1 mg/ml | 45.5 | 5 | 37 | 0.16% |
| 135, 136 | 48 | E. coli | | Avicel | 0.004 | 0.1 mg/ml | 48 | 5 | 37 | 0.23% |
| 207, 208 | 48 | E. coli | 2.2% | Avicel | 0.004 | 0.1 mg/ml | 45.5 | | | 0.03% |
| 209, 210 | 6 | E. coli | <3% | Avicel | 0.004 | 0.1 mg/ml | 45.5 | 7 | 37 | 0.11% |
| 211, 212 | 9 | E. coli | 10.1% | Avicel | 0.004 | 0.1 mg/ml | 45.5 | 7 | 37 | 0.11% |
| 211, 212 | 9 | E. coli | | Avicel | 0.004 | 0.1 mg/ml | 46 | 7 | 37 | 0.08% |
| 125, 126 | 5 | E. coli | <3% | Avicel | 0.004 | 0.1 mg/ml | 45.5 | | | 0.03% |
| 125, 126 | 5 | E. coli | | Avicel | 0.004 | 0.1 mg/ml | 48 | | | 0.02% |
| 97, 98 | 48 | P. pastoris | 5.3% | Avicel | 0.004 | 0.1 mg/ml | 47.75 | 5 | 55 | 0.89% |
| 101, 102 | 48 | P. pastoris | 4.6% | Avicel | 0.004 | 0.1 mg/ml | 47.75 | 5 | 55 | 0.96% |
| 193, 194 | 48 | E. coli | 6.2% | Avicel | 0.004 | 0.1 mg/ml | 45.5 | 7 | 37 | 0.15% |
| 193, 194 | 48 | E. coli | | Avicel | 0.004 | 0.1 mg/ml | 46 | 7 | 37 | 0.13% |
| 241, 242 | 48 | E. coli | <3% | Avicel | 0.004 | 0.1 mg/ml | 47.75 | 5 | 37 | 0.17% |
| 213, 214 | 5 | E. coli | 5.3% | Avicel | 0.004 | 0.1 mg/ml | 47.75 | 5 | 37 | 0.46% |
| 231, 232 | 9 | E. coli | 6.4% | Avicel | 0.004 | 0.1 mg/ml | 47.75 | 7 | 37 | 0.29% |
| 247, 248 | 9 | E. coli | 5.5% | Avicel | 0.004 | 0.1 mg/ml | 47.75 | 7 | 37 | 0.22% |
| 245, 246 | 9 | E. coli | 6.4% | Avicel | 0.004 | 0.1 mg/ml | 47.75 | 7 | 37 | 0.24% |
| 249, 250 | 5 | E. coli | 5.0% | Avicel | 0.004 | 0.1 mg/ml | 47.75 | 5 | 37 | 0.47% |
| 235, 236 | 6 | E. coli | | Avicel | 0.004 | 0.1 mg/ml | 48 | 7 | 37 | 0.31% |
| 263, 264 | 48 | E. coli | | Avicel | 0.004 | 0.1 mg/ml | 48 | 7 | 37 | 0.06% |
| 281, 282 | 6 | A. niger | 100 | Avicel | 0.004 | 0.1 mg/ml | 46 | 5 | 37 | 1.99% |
| 261, 262 | 5 | E. coli | <3% | Avicel | 0.004 | 0.1 mg/ml | 46 | 5 | 37 | 1.13% |
| 261, 262 | 5 | E. coli | 5.8% | Avicel | 0.004 | 0.1 mg/ml | 46 | 5 | 37 | 1.44% |
| 233, 234 | 5 | E. coli | | Avicel | 0.004 | 0.1 mg/ml | 48 | 7 | 37 | 0.18% |
| 219, 220 | 6 | E. coli | | Avicel | 0.004 | 0.1 mg/ml | 48 | 7 | 37 | 0.37% |
| 239, 240 | 6 | E. coli | | Avicel | 0.004 | 0.1 mg/ml | 48 | 5 | 55 | 0.52% |
| 217, 218 | 6 | E. coli | | Avicel | 0.004 | 0.1 mg/ml | 48 | 7 | 37 | 0.37% |
| 251, 252 | 45 | E. coli | <3% | Avicel | 0.004 | 0.1 mg/ml | 46 | 5 | 37 | 0.40% |
| 181, 182 | 6 | S. diversa | 4.3% | Avicel | 0.004 | 0.1 mg/ml | 44.5 | 7 | 37 | 0.63% |
| 181, 182 | 6 | S. diversa | 2.4% | Avicel | 0.004 | 0.1 mg/ml | 44.5 | 7 | 37 | 0.45% |
| 225, 226 | 6 | S. diversa | 4.4% | Avicel | 0.004 | 0.1 mg/ml | 44.5 | 5 | 37 | 1.21% |
| 229, 230 | 6 | S. diversa | 6.3% | Avicel | 0.004 | 0.1 mg/ml | 44.5 | 5 | 37 | 2.08% |
| 221, 222 | 6 | S. diversa | 3.3% | Avicel | 0.004 | 0.1 mg/ml | 44.5 | 5 | 55 | 1.08% |
| 223, 224 | 6 | S. diversa | 3.5% | Avicel | 0.004 | 0.1 mg/ml | 44.5 | 5 | 55 | 1.74% |
| 185, 186 | 6 | S. diversa | 7.9% | Avicel | 0.004 | 0.1 mg/ml | 44.5 | 5 | 37 | 1.34% |
| 139, 140 | 6 | S. diversa | 3.5% | Avicel | 0.004 | 0.1 mg/ml | 44.5 | 7 | 37 | 0.20% |
| 165, 166 | 6 | S. diversa | 5.5% | Avicel | 0.004 | 0.1 mg/ml | 44.5 | 5 | 55 | 0.84% |

### Exemplary Assays for the Initial Screening of Cellulases

### Exemplary Protocol for Single-Enzyme Digests (37° C. and 55° C.)

1. Preparation. For every 10 test samples, you'll usually need: two 96-well plates (for digests@37° C. and 55° C.), two clear 384-well plates (for corresponding timepoints) and space available on a 96-deep-well plate for enzyme dilutions.
2. Layout. Generally, 10 enzyme samples per plate plus positive and negative controls. Each sample gets 1 column. A typical screening layout is shown (certain details of this protocol are given for this layout).
   row B: 0.1 mg/mL enzyme@pH 5
   row C: 1.0 mg/mL enzyme@pH 5
   row D: 0.1 mg/mL enzyme@pH 7
   row E: 1.0 mg/mL enzyme@pH 7
   row F: 0.1 mg/mL enzyme@pH 9
   row G: 1.0 mg/mL enzyme@pH 9
3. Make 1.2× buffered substrate solutions. The following is for digestion at final concentrations of 0.4% AVICEL®, 50 mM buffer and 5 mM sodium azide. For every 12 samples to be tested (10 test plus 2 controls), you'll need approximately 10 mL each of the following buffered solutions. Sodium azide is added to inhibit growth of any microbial contaminants during digest reactions.
   a. 0.48% dispersed AVICEL®; 60 mM sodium acetate, pH 5.0; 6 mM sodium azide
   b. 0.48% dispersed AVICEL®; 60 mM sodium phosphate, pH 7.0; 6 mM sodium azide
   c. 0.48% dispersed AVICEL®; 60 mM sodium phosphate, pH 9.0; 6 mM sodium azide
4. Deposit 175 μL/well of 1.2× buffered substrate solutions into two 96-well plates. These will be the digest plates. Use a multichannel pipet. AVICEL® sinks rapidly, so each time you are pipetting out of the trough, pipet up and down to form an even suspension before transferring fluid to the 96-well plate. Array as shown in the layout above: pH 5 in rows B and C, pH 7 in rows D and E, pH 9 in rows F and G.
5. Prepare two 384-well timepoint plates with 35 μL/well stop solution. Use a TITERTEK™. Fill all wells of each plate with 35 μL stop solution.
6. Make 6× enzyme solutions. In a 96-well plate, make 0.6 mg/mL and 6 mg/mL dilutions of each enzyme stock. Make about 250 μL each of the two dilutions per sample. Dilute enzyme stocks with water and array them in two rows (upper row 0.6 mg/mL, lower row 6 mg/mL) of the plate in the order desired on the digest plates. Also include the positive and negative controls among the 12 samples.
7. Add enzyme to substrate and immediately take a t 0 timepoint. Using a multichannel pipet, remove 35 μL of 6× enzyme samples from the upper row of the dilution plate and transfer to rows B, D, and F of both digest plates (see layout above). Carefully pipet up and down to thoroughly mix, then immediately transfer 35 μL of each digest solution to the stop solution in the upper left quadruplet of the 384-well timepoint plate. Pipet up and down to mix with the stop solution. Note that if you're careful about minimizing pipetting error, for each row on the digest plate, you can use the same 12 pipet tips for both enzyme transfer and timepoint transfer. Store the 384-well timepoint plate with robo-lid at 4° C. until the next time point.
8. Incubate. Place one digest plate at 37° C. and the other at 55° C., using robo-lids and zip-lock bags humidified with a wet paper towel.
9. Take another time-point 3-5 hours later. First centrifuge the digest plates to pull down moisture condensed on the lids (3200×g for <1 minute). Using a multichannel set to 35 μL, pipet up and down to evenly suspend AVICEL®, then transfer 35 μL to the upper right quadruplet of the timepoint plate and mix with stop solution. Take care to minimize pipetting error. Note the length of digest for this timepoint.
10. Take a third timepoint at approximately 24 hours. On day 2, take a third timepoint as described above. Place in the lower left quadruplet and mix with stop solution. Note the length of digest for this timepoint.
11. Take a final timepoint at approximately 48 hours. On day 3, remove plates from incubators. Take a final timepoint as described above and mix with the stop solution in the lower right quadruplet of the timepoint plates. Note the length of digest and write it on the lids of the 96-well digest plates. Use the timepoint plate for the BCA and glucose oxidase assays. Apply a foil seal on the digest plates and store at −20° C. for later use in CE/HPLC analysis.

### Exemplary Protocol for the Glucose Oxidase β-Glucosidase) Assay

This exemplary protocol dilutes the digestion reactions 39-fold (including the 2-fold dilution of each time point into stop solution) and therefore is appropriate when you expect the concentration of glucose equivalents in the digestion products to range between around 25 to 400 μM. Higher glucose concentrations will also be detected but will fall beyond the linear range of the standards.

The following should be done for each 384-well stopped reaction plate (e.g., timepoint plate).
1. Make sure the APRICOT™ is set up and has the 384-well manifold attached and mounted with fresh tips.
2. Centrifuge stopped reaction plate at 2,000×g for a minute.
3. Add standard curves: The cellobiose (CB) standard is used to control for the β-glucosidase activity when performing the GO assay. When the β-gluc step is correctly carried out, the CB standard, as expressed in glucose equivalents, should give the same slope as the glucose standard.
   a. It's convenient to make the dilutions in a 96-deep-well plate so that they will be arrayed as desired for transfer to the assay plates.
   b. Standards can be diluted in water.
   c. Make a 200 μM CB solution and a 400 μM glucose solution. Perform 4 successive 2-fold dilutions of each. With the inclusion of a 0 μM solution, you'll have a 6-point standard curve for both glucose and CB.
   d. An exemplary protocol:
      i. In one well of a 96-deep-well plate, make 1 mL of a 200 μM CB solution. In another well, make 1 mL of a 400 μM glucose solution.
      ii. Add 0.5 mL water to each of 5 wells adjacent to each standard in order to make successive dilutions.
      iii. Make 4 successive 2⁻¹ dilutions for each standard (0.5 mL+0.5 mL water). Remember to leave the final well without the standard as the “0 μM” point.
   e. Transfer 35 μL/well of each dilution to the 35 μL stop solution to unused areas of the timepoint plate like rows A, B, O and P. Load each standard in quadruplicate.
4. Using a TITERTEK™ MULTIDROP™, load 35 μL/well of freshly made β-glucosidase Solution into a black 384-well plate.
5. Using the APRICOT™, transfer 4 μL/well from the timepoint plate to the β-glucosidase plate and mix; (an exemplary Apricot program for these transfer steps is called DYCAICO7™). Once mixed, the final pH should be around 7.5. Store the timepoint plate at −20° C. with foil seal in case either assay needs repeating.
6. Centrifuge plate 2000×g for a few seconds to eliminate air bubbles.
7. Incubate the β-glucosidase plates at room temperature for 2-3 hours.
8. Use a TITERTEK MULTIDROP™ to add 40 μL/well of freshly-made 2× GO Assay Solution to the black β-glucosidase-treated plate. (Make this solution immediately before you plan to use it.)
9. Incubate the assay plate at room temperature, protected from light, for 30 minutes. (If necessary, centrifuge plate 2000×g for a few seconds to eliminate air bubbles.)
10. Read fluorescence at 530/595 nm for resorufin detection.
11. Save data as a text file for analysis using Excel template.

### Exemplary Protocol for the BCA Assay

1. Preheat BEAVER™ (Tansun Limited, UK) heater: bottom=70° C., top=75° C.
2. Make sure the APRICOT™ is set up and has the 384-well manifold attached. Run the program several times to get the washer fully primed.
3. Standards: Add standards to the timepoint plate as described for the GO assay. Note that in the past, I used 0.1 mg/mL protein solution (like BSA) for diluting the standards in order to mimic the protein background present in the test samples in the BCA assay. This is only relevant when using high levels of relatively impure protein in the digests.
4. Centrifuge timepoint plate at 3200×g for 5 minutes at 4° C. to pellet AVICEL® present in the samples.
5. For each timepoint plate, use two new clear 384-well plates for BCA assay (duplicate plates). So you'll need 4 plates total to cover both 37° and 55° timepoints.
6. Using a TITERTEK MULTIDROP™, add 15 μL/well of freshly combined A+B solution to each assay plate.
7. Using the APRICOT™, transfer 15 μL enzyme digest supernatant from the timepoint plate to the assay plate in duplicate. Avoid transferring AVICEL® (suspended AVICEL® will cause background). Make sure there is a mixing step on the APRICOT™. Since there is space for only two plates on the BEAVER™ heater, you should stagger the 37° and 55° timepoint assays by about 40 minutes.
8. Immediately apply foam lids to assay plates and place in the BEAVER™ heater.
9. Incubate for exactly 35 minutes.
10. Once incubation is complete, immediately place plates on ice, replace foam lids with original lids, and quickly head toward the centrifuge.
11. Centrifuge 3200×g for 5 minutes at 4° C. This centrifugation at 4° C. will rapidly cool the plates.
12. Read absorbance at 562 nm.
13. Save data as a text file for analysis using EXCEL™ template.

### Solutions:

### 13% Dispersed AVICEL®

Measure 130 grams AVICEL® microcrystalline cellulose (FMC Biopolymer: type PH 105, grade NF/EP) into a clean, high-speed blender and add 18 MΩ dI water to the 1 L measuring line (about 916 mL). Close the lid and blend at highest speed for 20 minutes. Transfer the suspension to an autoclave-safe container and autoclave using the 30 minute “liquid” cycle. Store at room temperature.

### Stop Solution

(400 mM carbonate buffer, pH 10)

Follow directions below for BCA Solution A but do not add the BCA component. Then dilute 2-fold to produce 1 liter of 400 mM carbonate solution.

### BCA Solution A

(5 mM BCA in 800 mM carbonate pH 10)

| | | | final |
|---|---|---|---|
| | FW | amt | concentration |
| sodium carbonate, | 124.00 | 64 mg/mL | 516 mM |
| monohydrate* | | | |
| sodium bicarbonate | 84.01 | 24 mg/mL | 286 mM |
| bicinchoninic acid disodium | 388.28 | 1.95 mg/mL | 5 mM |
| salt hydrate (Sigma D8284) | | | |

1. In a 500 mL beaker with stir bar, combine 32 grams sodium carbonate monohydrate* with 12 grams sodium bicarbonate.
2. Add 18 MΩ water to about 450 mL and place on a magnetic stirrer until completely dissolved (about 15-30 minutes).
3. Add 975 mg BCA reagent and continue stirring until completely dissolved.
4. Adjust volume to 500 mL with 18 MΩ water.
5. Sterile filter.
6. Store at 4 ° C. Make fresh every 2-3 weeks.
   Alternatively, use 27.35 grams of anhydrous sodium carbonate (FW 106) in step 1 above.

### BCA Solution B

| | FW | amt | final concentration |
|---|---|---|---|
| cupric sulfate pentahydrate | 249.69 | 1.24 mg/mL | 5 mM |
| (Sigma C2857) | | | |
| L-serine | 105.09 | 1.26 mg/mL | 12 mM |

1. In a 500 mL beaker with stir bar, combine 620 mg cupric sulfate pentahydrate with 630 mg L-serine.
2. Add 18 MΩ water to about 450 mL and place on a magnetic stirrer until completely dissolved (about 15-30 minutes).
3. Adjust volume to 500 mL with 18 MΩ water.
4. Sterile filter.
5. Store at 4 ° C. Make fresh every 2-3 weeks.

### ≈-Glucosidase Solution

| Component | [Stock] | [final] |
|---|---|---|
| Sterile dI water | n/a | qs |
| Na Phosphate Buffer, pH 7.0* | 500 mM | 125 mM |
| SEQ ID NO: 424** β-glucosidase | variable | 0.04 U/mL |

*Once the high-pH solution from the timepoint plate is diluted 10-fold into β-glucosidase solution, the final pH should be around 7.5, which is appropriate for SEQ ID NO: 424.
**A His-tagged version of this enzyme can also be used.

### 2× GO Assay Solution

| Component | [Stock] | | 2x Cocktail | |
|---|---|---|---|---|
| Sterile dI water | n/a | | qs | |
| Na Phosphate Buffer, pH 7.4 | 500 | mM | 100 | mM |
| GO/HRP mix | 2500/250 | U/mL | 2/0.2 | U/mL |
| Amplex Red | 50 | mM | 0.1 | mM |

Add components in the order listed, then use immediately in the assay.

### GO/HRP Mix

Glucose oxidase: Sigma #G7141-50KU. Dissolve all 50,000 units in 5 mL 50 mM phosphate, pH 7.4 buffer.
Horseradish peroxidase: Sigma #P2088-5KU. Dissolve all 5,000 units in 5 mL 50 mM phosphate, pH 7.4 buffer.

Combine Glucose oxidase and HRP solutions. Use a sterile syringe to add an equal volume (10 mL) of sterile glycerol. Mix well. This gives 20 mL of solution with final concentrations: 2,500 U/mL GO; 250 U/mL HRP. Aliquot into 1-mL tubes and store at −20° C.

### 50 mM Amplex Red

Molecular Probes #A22177 (10×10-mg vials; MW=257.25). Dissolve each 10 mg vial in 0.777 mL DMSO to produce a 50 mM stock. Store at −20° C. protected from light.

Optional 10 mM stock: Invitrogen #A12222; MW=257.25. Dissolve all 5 mg in 1.94 mL DMSO. Aliquot 250 μL per 0.5-mL tube and freeze at −20° C. protected from light.

### Sodium Azide

Sigma #S2002 (FW 65.01); Be careful; it's toxic and carcinogenic. Make concentrated stocks in water (e.g., 1 M). For use as an antimicrobial additive, typical concentrations are 0.02-0.05% (w/v), which is between 3 and 8 mM.

### Exemplary Enzyme Digestibility Assay-Large Scale

Large scale enzyme digestibility assays can also be used to identify an enzyme of the invention, and to characterize an enzyme of the invention. An exemplary large scale enzyme digestibility assay is:

The exemplary large scale enzyme digestibility assay was carried out in a 10 mL glass crimp-top vial. The moisture content and the sugar composition were determined before the assay. 250 dw mg±10 mg of shredded bagasse was weighed into the glass vial and certain amount of 100 mM NaOAc buffer was added depending on the final enzyme concentration. The NaOAc buffer had pH of 5.0, and it contained 10 mM of NaN₃ as a growth inhibitor. The bagasse mixture was capped without sealing and preheated in a 37° C. incubator for about 20 min before adding the proper amount of enzyme solution. The enzyme loading in the large scale reaction is 25 mg of total protein/g of cellulose. The total reaction volume is 5 mL. Once the enzyme solution was added, the vials were sealed and clamped to the rotary. About 200 uL of sample was drawn at 2, 4, 6 and 24 hr. The sample was centrifuged at 13,200 rpm for 5 min. The supernatant was diluted 4 folds into a 384-well plate. The sugar composition in the reaction product was analyzed with RI-HPLC against known standards and cellulose conversion was calculated based on the theoretical cellulose value in the bagasse.

### Example 6

### Identification and Characterization of Enzymes of the Invention

This example describes exemplary strategies for identifying and characterizing enzymes of the invention, which include enzymes to be used in the mixtures (“cocktails”) of enzymes of the invention designed to efficiently process (“hydrolyze”) the complex structures of various biomass, e.g., sugarcane plant fibers (also called “bagasse”), which require multiple enzyme activities to completely degrade (“hydrolyze”) of the bagasse.

In alternative aspects, different combinations of enzymes are tested to determine the optimum combination necessary to hydrolyze a bagasse substrate (or any other target biomass substrate) to a desired level. Categorization of enzymes can be based on their previously determined activity on model substrates, and not necessarily their sequence identity (sequence similarity to/ homology to) known enzymes. For example, an enzyme that releases cellobiose from cellulose will be considered a cellobiohydrolase even if by sequence it is most similar to an endoglucanase.

### Enzyme Discovery:

Prokaryotic Enzymes:

Many of known enzymes have been discovered from prokaryotic libraries by functional screening on unlabeled or labeled substrates, e.g., unlabeled or labeled AVICEL™. Functional screening can also comprise the use of any assay or protocol, e.g., xylan traps, and the like. Gene libraries from environmental-derived samples, including soil, air or water samples, e.g., from agricultural fields, such as sugarcane fields, and including any microorganism found in any environmental-derived sample, either directly or indirectly: e.g., plant (e.g., sugarcane, corn) microorganisms; insect-associated (e.g., termite gut) microorganisms; animal-associated (e.g., ruminant gut) microorganisms; and the like, are used to express polypeptides, which in turn are screened for a lignocellulosic activity, e.g., a glycosyl hydrolase, cellulase, endoglucanase, cellobiohydrolase and/or β-glucosidase (beta-glucosidase) activity, which includes for example bagasse-degrading or corn fiber- (e.g., corn seed fiber)-degrading activity.

Fungal Enzymes:

Functional screening will include fungal libraries in addition to bacterial libraries; enzymes having a lignocellulosic activity—including a biomass-degrading, e.g., a bagasse-degrading or corn seed fiber-degrading, activity will be identified from fungal sources (many of the best performing biomass-degrading enzymes have been identified from fungi, so this may represent a good source for bagasse specific fibrolytic enzymes).

Fungi that efficiently degrade bagasse presumably have repertoire of enzymes that have evolved to work well together. It is possible that multiple bagasse-degrading enzymes from the same organism synergize in a bagasse degrading application when compared to enzymes isolated from different organisms. For these reasons, this exemplary enzyme discovery strategy of this invention focuses on fungal genes.

In one embodiment, the discovery of fungal bagasse degrading enzymes utilizes Verenium Corporation's High Throughput Culturing technology to isolate and array unique microbes from environmental samples. A combination of approaches can be evaluated to identify the enzymes secreted by fungi as they are actively degrading and growing on a biomass substrate, e.g., a bagasse or corn fiber (e.g., corn seed fiber) substrate. In one embodiment, isolating most or all of the fibrolytic enzymes from a highly active fungus provides an effective combination of enzymes that synergize well together when heterologously expressed in plants or microbes.

Substrate Pretreatment:

Initially, simple bench scale pretreatments of the biomass substrate, e.g., bagasse or corn fiber substrate, is performed and evaluated.

Exemplary Enzyme Evaluations in Application Assays

In one embodiment, an application assay will be used to determine the activity of enzymes and enzyme combinations on untreated and pretreated biomass substrate, e.g., bagasse or corn fiber (e.g., corn seed fiber) substrate. This can involve utilizing analytical techniques to quantitate and identify the sugars released from the biomass substrate. The methods established to determine the exact sugar composition of the fiber substrate also can be applied on the biomass (e.g., bagasse or corn fiber) sample. This information can be used to determine the extent of degradation of the substrate after enzyme treatment.

The best combination of enzymes will be systematically determined. This strategy can include fixing the identity of one or more enzymes or pretreating the substrate. Benchmark enzymes (commercial preparations and/or subclones of existing fibrolytic enzymes) can be used to develop and validate this assay.

For enzymes like CBH and beta-glucosidases, which are severely inhibited by cellobiose and glucose respectively, product inhibition may be measured.

A. Enzyme Discovery
a. Exemplary functional screening strategies for prokaryotic enzymes
   Using functional assays, prokaryotic gene libraries from relevant sample site(s) (e.g., corn or sugarcane fields) are screened for target enzyme activities (e.g., glycosyl hydrolases, etc.).
   Enzyme screens with different fluorophore (e.g. 4-Methyl-umbelliferyl and Resorufin-linked substrates) can be multiplexed to screen for multiple enzyme activities at the same time.
b. Exemplary screening strategies for fungal strains.
   Screen novel fungi from High Throughput Culturing, and top fungal strains identified during the corn seed fiber project for strains that degrade target biomass (e.g., corn fiber or bagasse) in vivo effectively.
   The identity and diversity of the identified strains can be determined by conducting an 18S analysis.
   Generate full-length cDNA expression libraries from cultures actively producing biomass-degrading (e.g., bagasse-degrading) enzymes.
   Screening efforts can be applied to clone most or all of the genes present in the culture media from these strains—particular if they are actively degrading the target biomass (e.g., corn fiber or bagasse). Exemplary approaches that can be used to clone all these genes may involve a combination of the following methods:
      1. Screen cDNA libraries by (a) a sequence based approach, and/or, (b) screened gDNA libraries by substrate-binding domain (SBD), put genomic clones directly in Aspergillus, intron splicing done by the host in process of creating cDNA versions to confirm spliced clones;
      2. Proteomic analysis to identify proteins secreted into the culture media when these strains are growing on the target biomass (e.g., corn fiber or bagasse) substrate.

B. Exemplary Enzyme Characterizations
a. Bioinformatic characterization of newly discovered genes
   Domain structure, enzyme class and family
   Signal sequences, rare codons, etc
b. Subclone newly discovered genes and optimize protein expression for characterization and application testing.
c. Enzyme characterization of all subclones with a standard protocol
   specific activity for selected, or all, enzyme preparations can be determined on a model substrate; this information can be used for calculating enzyme loading in applications assays.

C. Exemplary Enzyme Evaluations in Application Assays
a. Alternative Assay Development strategies:
   Distribution of target biomass (e.g., corn fiber or bagasse) substrate;
   Reaction format conditions;
   Medium throughput assay to determine total sugar released (e.g., a reducing sugar assay);
   Detailed analysis (HPLC/ELSD, LC/MS, etc) can be performed on samples with high levels of sugar release; methods to quantitate and identify the sugars released and what remains undigested can be applied. In one embodiment: quantitate the sugar composition of the fiber substrate in order to evaluate enzyme performance as a % sugar released;
   Exemplary strategy for combinatorial evaluation of enzymes: may include enzymatic or chemical pretreatment for early studies on specific enzyme classes.
b. Validation of assay with benchmark enzymes: this will give some target performance criteria and information on units of enzyme to add in application assay.
c. Evaluation of all enzymes in application assay: identify enzymes and enzyme combinations that result in the most sugar release from the target biomass (e.g., corn fiber or bagasse) substrate.

D. Evaluation of Enzymes in Plant Cells—Transgenic Plants

In one embodiment, promising candidates in each hydrolytic class are subjected to plant expression, including both in host plant cells, plant tissues and/or transgenic plants. These plant-expressed enzymes (e.g., hydrolases) will be tested in target biomass (e.g., corn fiber or bagasse) applications assays.

E. Evolution—Optimization of Enzyme Activity

In one embodiment, and depending on enzyme performance, some or all of the enzymes are “optimized”, i.e., are sequence-modified to improve parameters such as enzyme productivity on a given substrate, temperate optimum, pH optimum, stability, specific activity, product inhibition, etc. Optimization may involve evolution using Verenium Corporation's proprietary GENE SITE SATURATION MUTAGENESIS (or GSSM) and/or GENEREASSEMBLY™ technologies.

### Example 7

### Multidomain Enzymes of This Invention

This example describes inter alia the designing and making of multi-domain polypeptides (e.g., enzymes) (and the nucleic acids that encode them) of this invention.

The invention provides lignocellulosic enzymes, e.g., a glycosyl hydrolase, cellulase, endoglucanase, cellobiohydrolase, beta-glucosidase, xylanase, mannanse, β-xylosidase and/or arabinofuranosidase enzymes, that are multidomain enzymes comprising at least one (e.g., can include multiple) carbohydrate binding module(s), which can be a heterologous or homologous carbohydrate binding module (CBM), and can be any known CBM module, e.g., a cellulose binding module, a lignin binding module, a xylose binding module, a mannanse binding module, a xyloglucan-specific module, an arabinofuranosidase binding module, etc., from another lignocellulosic enzyme. This example described exemplary protocols for the routine identification of carbohydrate binding modules, e.g., cellulose binding modules.

### CBM Binding Assay Materials:

The substrates for an exemplary CBM binding assay (for cellulose and xylan) are AVICEL® microcrystalline cellulose (MCC) and Oat-Spelt Xylan (Sigma, X-0627). Cellulose binding modules are either purified CBM-GST fusion proteins or the lysates containing CBMs. BSA was chosen as control along the assays.

### CBM Binding Assay Protocol:

CBM binding assays were done in 1.5 ml Eppendorf tubes with total 200 ul of reaction volume in which contained 1 mg of substrates (Avicel MCC or Oat_Spet-Xylan) and 0.1mg of CBM in 50 mM TisHCl buffer (pH 8.0). After the reactions were done at room temperature for 1 h, the unbound CBMs remaining in the supernatants were separated from the bound CBMs remaining in the pellets by centrifugation at 10,000 rpm for 5 min. Next, washed the pellets for three times with same buffer, added SDS loading buffer and boiled for 10min to release the bound CBMs from the pellets. Finally, the bound and unbound CBMs were detected by SDS PAGE.

### CBM Definition:

As noted above, any carbohydrate binding modules (CBM), e.g., cellulose binding module, can be incorporated into an enzyme of this invention, and many such modules are well known in the art, for example, in a Carbohydrate Active Enzymes website a carbohydrate-binding module is defined as contiguous amino acid sequence within a carbohydrate-active enzyme with a discreet fold having carbohydrate-binding activity. A few exceptions are CBMs in cellulosomal scaffolding proteins and rare instances of independent putative CBMs. The requirement of carbohydrate binding modules, e.g., cellulose binding modules, existing as modules within larger enzymes sets this class of carbohydrate-binding protein apart from other non-catalytic sugar binding proteins such as lectins and sugar transport proteins.

CBMs were previously classified as cellulose-binding domains based on the initial discovery of several modules that bound cellulose. However, additional modules in carbohydrate-active enzymes are continually being found that bind carbohydrates other than cellulose yet otherwise meet the CBM criteria, hence the need to reclassify these polypeptides using more inclusive terminology. Previous classification of cellulose-binding domains were based on amino acid similarity. Groupings of CBDs were called “Types” and numbered with roman numerals (e.g. Type I or Type II CBDs).

In keeping with the glycoside hydrolase classification, these groupings are now called families and numbered with Arabic numerals, as noted below. In alternative embodiment, enzymes of the invention include one or multiple members of any or all of these carbohydrate binding modules, e.g., cellulose binding modules, as domains linked to, e.g., as sequences spliced into or added onto, any enzyme or peptide of the invention:

CBM_{—}1:

Modules of approx. 40 residues found almost exclusively in fungi. The cellulose-binding function has been demonstrated in many cases, and appears to be mediated by three aromatic residues separated by about 10.4 angstrom and which form a flat surface. The only non-fungal occurrence of CBM1 is in an algal non-hydrolytic polysaccharide-binding protein which is composed of four repeated CBM1 modules. Binding to chitin has been demonstrated in one case.

CBM_{—}2 (CBM_{—}2a, CBM_{—}2b)

Modules of approx. 100 residues and which are found in a large number of bacterial enzymes. The cellulose-binding function has been demonstrated in many cases. Several of these modules have been shown to also bind chitin or xylan.

CBM_{—}3 (CBM_{—}3a, CBM_{—}3b, CBM_{—}3c)

150 residues found in bacterial enzymes. The cellulose-binding function has been demonstrated in many cases. In one instance binding to chitin has been reported.

CBM_{—}5_{—}12:

approx. 40-60 residues. The majority of these modules is found among chitinases where the function is chitin-binding. Distantly related to the CBM5 family.

CBM_{—}10:

Modules of approx. 50 residues. The cellulose-binding function has been demonstrated in one case.

Results of these carbohydrate binding module (CBM) assay determinations are shown in Table 5 and Table 6, below. These results, as summarized in Tables 5 and 6, demonstrate the presence of functional CBMs in some of the exemplary polypeptides of the invention. The tables also indicate activity associated with these CBMs of the invention.

The invention provides chimeric polypeptides, including enzymes, comprising polypeptide sequences of the invention (e.g., the enzymes of the invention, including enzymatically active subsequences (fragments) of polypeptides of the invention), including any combination of CBMs, including the exemplary CBMs noted below. For example, a chimeric polypeptide of the invention having lignocellulosic activity, e.g., a glycosyl transferase, a cellulase, a cellulolytic activity, an endoglucanase, a cellobiohydrolase, a beta-glucosidase, a xylanase, a mannanse, a β-xylosidase or an arabinofuranosidase activity, can comprise one, two or several heterologous, or endogenously rearranged, CBMs; and these heterologous or endogenously rearranged CBMs can be positioned internal to the sequence, and/or amino terminal or carboxy terminal to an amino acid sequence; if the chimeric polypeptide of the invention is a recombinant protein, then the chimeric polypeptide can be made by constructing a recombinant chimeric coding sequence encoding the flanking and/or internal heterologous or endogenously rearranged CBMs coding sequences; and these chimeric nucleic acid coding sequences are also sequences of the invention.

TABLE 5
CBM in trans Subclones

| | | CBM | CBM | | |
|---|---|---|---|---|---|
| | | Binding | Binding to | | |
| CBM | Parental clone (which includes | to Avicel | Oat-Spelt | Family/Other Domains Included in | Comments for |
| family | indicated CBM) | MCC | Xylan | clone | ORF Subclone |
| 2a | SEQ ID NO: 468 (encoded by, | .+ | .+? | Glycosyl Hydrolase family 8 + 2CBM2 | signal removed |
| | e.g., SEQ ID NO: 467) | | | | |
| 2a | SEQ ID NO: 468 (encoded by, | .+ | .− | Glycosyl Hydrolase family 8 + 2CBM2 | signal removed |
| | e.g., SEQ ID NO: 467) | | | | |
| 2a | SEQ ID NO: 470 (encoded by, | .+ | .− | CBM2 + Cellulase | signal removed |
| | e.g., SEQ ID NO: 469) | | | | |
| 2a | SEQ ID NO: 6 (encoded by, | .+ | .− | Glycosyl Hydrolase family 6 + CBM2 | none/change start |
| | e.g., SEQ ID NO: 5) | | | | |
| 2a | SEQ ID NO: 464 (encoded by, | .++ | .+? | CBM2 + CBM10 + Cellulase | signal removed |
| | e.g., SEQ ID NO: 463) | | | | |
| 3b | SEQ ID NO: 438 (encoded by, | .+ | .− | CBM3-Fn3-CBM5 | None |
| | e.g., SEQ ID NO: 437) | | | | |
| 3b | SEQ ID NO: 94 (encoded by, | .+ | .−? | F. 48-DUF-CBM3 | leader removed |
| | e.g., SEQ ID NO: 93) | | | | |
| 3b | SEQ ID NO: 176 (encoded by, | .+ | .− | F.48-CBM3 | None |
| | e.g., SEQ ID NO: 175) | | | | |
| 10 | SEQ ID NO: 12 (encoded by, | .+ | .− | Cellulase + CBM5, 10 + F6 | remove leader |
| | e.g., SEQ ID NO: 11) | | | | |
| 10 | SEQ ID NO: 464 (encoded by, | .+ | .− | CBM2 + CBM10 + Cellulase | signal removed |
| | e.g., SEQ ID NO: 463) | | | | |
| 17_28 | SEQ ID NO: 8 (encoded by, | .+ | .+ | F5 + 2CBM_17_28 | None |
| | e.g., SEQ ID NO: 7) | | | | |
| 17_28 | SEQ ID NO: 10 (encoded by, | .+ | .+ | F5 + CBM17 + 3SLH | Signal Removed |
| | e.g., SEQ ID NO: 9) | | | | |
| 17_28 | SEQ ID NO: 430 (encoded by, | .+ | .+ | F5 + CBM17 + 3SLH | None |
| | e.g., SEQ ID NO: 429) | | | | |
| 3b or | SEQ ID NO: 448 (encoded by, | .++ | .+ | Cellulase + CBM3 | |
| 3c | e.g., SEQ ID NO: 447) | | | | |
| 3b or | SEQ ID NO: 466 (encoded by, | .++ | .+ | Cellulase + CBM3 | |
| 3c | e.g., SEQ ID NO: 465) | | | | |
| 3b or | SEQ ID NO: 2 (encoded by, | .+ | .++ | Cellulase + Glycosyl Hydrolase family | |
| 3c | e.g., SEQ ID NO: 1) | | | 6 + CBM3 | |
| 3b or | SEQ ID NO: 428 (encoded by, | .+ | .+ | Big_2 + Glycosyl Hydrolase family | |
| 3c | e.g., SEQ ID NO: 427) | | | 9 + CBM3 | |
| 3b or | SEQ ID NO: 446 (encoded by, | .− | .+ | Glycosyl Hydrolase family 9 + CBM3 | |
| 3c | e.g., SEQ ID NO: 445) | | | | |
| 3b or | SEQ ID NO: 440 (encoded by, | .− | .−? | Glycosyl Hydrolase family 10 + CBM3 | |
| 3c | e.g., SEQ ID NO: 439) | | | | |
| 3b or | SEQ ID NO: 448 (encoded by, | .− | .−? | Cellulase + CBM3 | |
| 3c | e.g., SEQ ID NO: 447) | | | | |
| 4_9 | SEQ ID NO: 462 (encoded by, | .− | .+? | Glycosyl Hydrolase family | |
| | e.g., SEQ ID NO: 461) | | | 16 + CBM4_9 | |
| 4_9 | SEQ ID NO: 436 (encoded by, | .− | .+ | Glycosyl Hydrolase family | |
| | e.g., SEQ ID NO: 435) | | | 16 + 2CBM4_9 | |
| 4_9 | SEQ ID NO: 436 (encoded by, | .− | .+ | Glycosyl Hydrolase family | |
| | e.g., SEQ ID NO: 435) | | | 16 + 2CBM4_9 | |
| 4_9 | SEQ ID NO: 442 (encoded by, | .−? | .+? | DUF1083 + Glycosyl Hydrolase family | |
| | e.g., SEQ ID NO: 441) | | | 10 + He_PIG + CBM4_9 | |
| 4_9 | SEQ ID NO: 444 (encoded by, | .− | .+ | Dockerin_1 + Esterase | |
| | e.g., SEQ ID NO: 443) | | | GH10 + CBM4_9 | |
| 4_9 | SEQ ID NO: 432 (encoded by, | .− | .+ | Glycosyl Hydrolase family | |
| | e.g., SEQ ID NO: 431) | | | 11 + CBM4_9 | |
| 4_9 | SEQ ID NO: 434 (encoded by, | .− | .−? | Glycosyl Hydrolase family | |
| | e.g., SEQ ID NO: 433) | | | 10 + CBM4_9 | |
| 5_12 | SEQ ID NO: 438 (encoded by, | .− | .−? | 2Fn3, CBM_5_12 | |
| | e.g., SEQ ID NO: 437) | | | | |
| 5_12 | SEQ ID NO: 452 (encoded by, | .− | .−? | Glycosyl Hydrolase family | |
| | e.g., SEQ ID NO: 451) | | | 19 + PKD + CBM5_12 | |
| 6 | SEQ ID NO: 454 (encoded by, | .− | .−? | Glycosyl Hydrolase family 45 + CBM6 | |
| | e.g., SEQ ID NO: 453) | | | | |

TABLE 6
Non-Glycosyl Hydrolase (GH)-associated CBMs

| Amino Acid SEQ ID NO: | | | CBM Binding to | CBM Binding to Oat- |
|---|---|---|---|---|
| (encoded by SEQ ID NO:) | Domains Included | Comments | Avicel MCC | Spelt Xylan |
| SEQ ID NO: 450 (encoded by, | CBM_33 + CBM_2 | signal removed | .+ | .− |
| e.g., SEQ ID NO: 449) | | | | |
| SEQ ID NO: 472 (encoded by, | CBM_33 + CBM_2 | none | .+ | .− |
| e.g., SEQ ID NO: 471) | | | | |
| SEQ ID NO: 438 (encoded by, | CBM_5_12 + 2Fn3 + | none | .+ | .− |
| e.g., SEQ ID NO: 437) | CBM_3 | | | |
| SEQ ID NO: 458 (encoded by, | CBM_33 + Fn3 + | signal removed | .+ | .−? |
| e.g., SEQ ID NO: 457) | CBM_2a | | | |
| SEQ ID NO: 456 (encoded by, | CBM_2a | none | .+ | .−? |
| e.g., SEQ ID NO: 455) | | | | |
| SEQ ID NO: 266 (encoded by, | Fn3 homolog | leader removed | .−? | .−? |
| e.g., SEQ ID NO: 265) | | | | |
| SEQ ID NO: 460 (encoded by, | CBM_33 (CBP21 | native | .−? | .−? |
| e.g., SEQ ID NO: 459) | homolog) | | | |

### Example 8

### Monocot and Dicot Optimized Genes of this Invention

This example describes, inter alia, the design and making of nucleic acid sequences of the invention designed, or “optimized”, for optimal expression in a dicot and/or a monocot cell and/or plant.

Dicot and monocot plant synthetic genes were designed using the backtranslation program in Vector NTI 9.0™. Four protein sequences were back-translated into monocot optimized and dicot optimized coding sequences using the preferred codons for monocots or dicots. Additional sequence was added to the 5′ and 3′ end of each cellulase gene coding sequence for cloning and differential targeting to subcellular compartments. These sequences included a BamHI cloning site, Kozak sequence, and N-terminal signal sequence at the 5′ end. Vacuolar or ER targeting sequences, and a SacI cloning site was added at the 3′ end. Silent mutations were introduced to remove any restriction sites which interfered with cloning strategies. Synthetic genes were synthesized by GENEART™ (Germany).

The dicot optimized gene encoding the exemplary SEQ ID NO:360 (a CBH1 protein) is SEQ ID NO:480; the dicot optimized gene encoding the exemplary SEQ ID NO:358 (a CBH2 protein) is SEQ ID NO:481; the dicot optimized gene encoding the exemplary SEQ ID NO:168 (an endoglucanase) is SEQ ID NO:482; and, the dicot optimized gene encoding the exemplary SEQ ID NO:34 (a CBH1 protein) is SEQ ID NO:487. The monocot optimized gene encoding the exemplary SEQ ID NO:360 is SEQ ID NO:483; the monocot optimized gene encoding the exemplary SEQ ID NO:358 is SEQ ID NO:484; the monocot optimized gene encoding the exemplary SEQ ID NO:168 is SEQ ID NO:485; and the monocot optimized gene encoding the exemplary SEQ ID NO:34 is SEQ ID NO:486.

### Example 9

### Construction of Plant Expression Vectors

The invention provides various plant expression systems, including vectors, recombinant viruses, artificial chromosomes and the like, comprising nucleic acids of this invention, including nucleic acids encoding enzymes of this invention, and including sequences complementary to the enzyme-encoding sequences; and this example describes making some of these embodiments.

Expression vectors capable of directing the expression of cellulases in transgenic plants were designed for both monocot and dicot optimized cellulases. Tobacco expression vectors used the constitutive promoter Cestrum yellow leaf curl virus (CYLCV) promoter plus leader sequence (SEQ ID NO:488) to drive expression of the dicot optimized cellulase genes. Tobacco expressed cellulases were targeted to the endoplasmic reticulum (ER) via fusion to the Glycine max glycinin GY1 signal sequence (SEQ ID NO:473) and the ER retention sequence (SEQ ID NO:474). Tobacco expressed cellulases were targeted to the vacuole via fusion of the cellulase gene with the sporamin vacuolar targeting sequence (SEQ ID NO:475) at the C-terminus (Plant Phys 1997: 114, 863-870) and the GY1 signal sequence at the N-terminus. Plastid targeting of the cellulase was via the transit peptide (SEQ ID NO:476) from ferredoxin-NADP+ reductase (FNR) of Cyanophora paradoxa fused to the N-terminus (FEBS Letters 1996: 381, 153-155).

The Glycine max glycinin GY1 promoter and signal sequence (GenBank Accession X15121) was used to drive soybean seed specific expression of cellulases. Targeting of the cellulase in soybean involved either the C-terminal addition of ER retention sequence (SEQ ID NO:474) or protein storage vacuole (PSV) sequence, (SEQ ID NO:477), from β-conglycinin (Plant Phys 2004:134, 625-639).

The maize PepC promoter (The Plant Journal 1994: 6(3), 311-319) was used to drive maize leaf specific expression of each monocot optimized cellulase. The cellulase gene was fused to the gamma zein 27 kD signal sequence (SEQ ID NO:478) at the N-terminus to target through the ER and fused to the vacuole sequence domain (VSD) from barley polyamine oxidase (SEQ ID NO:479) to direct the cellulase into the leaf vacuole (Plant Phys 2004: 134, 625-639). Alternatively the ER retention sequence (SEQ ID NO:474) was used in place of the VSD to retain the cellulase in the ER. Plastid targeted constructs contained the FNR transit peptide described above. Each of the maize optimized cellulases was cloned behind the rice glutelin promoter for expression in the endosperm of the maize seed. As described above, additional sequences were added for targeting of the protein to the ER or the endosperm. Vector component information is shown in Table 7. All expression cassettes were subcloned into a binary vector for transformation into tobacco, soybean, and maize using recombinant DNA techniques that are known in the art.

### Table 7. Plant Expression Vectors Used for Transgenic Tobacco, Maize, and Soybean Event Production.

The invention provides various plant expression systems, including vectors, recombinant viruses, artificial chromosomes and the like, comprising nucleic acids of this invention, including nucleic acids encoding enzymes of this invention, and including sequences complementary to the enzyme-encoding sequences, for use in various specific plants, including tobacco, maize (corn) and/or soybean; and this example describes making some of these embodiments.

| | | | Subcellular | Construct |
|---|---|---|---|---|
| Crop | Enzyme (Enzyme Class) | Promoter | Targeting | number |
| tobacco | SEQ ID NO: 360 (encoded by SEQ ID | Constitutive | Vacuolar | 15935 |
| | NO: 480) (CBH1) | (CYLCV) | | |
| tobacco | SEQ ID NO: 360 (encoded by SEQ ID | Constitutive | ER | 15936 |
| | NO: 480) (CBH1) | (CYLCV) | | |
| tobacco | SEQ ID NO: 360 (encoded by SEQ ID | Constitutive | Plastid | 17024 |
| | NO: 480) (CBH1) | (CYLCV) | | |
| tobacco | SEQ ID NO: 358 (encoded by SEQ ID | Constitutive | ER | 17022 |
| | NO: 481) (CBH2) | (CYLCV) | | |
| tobacco | SEQ ID NO: 358 (encoded by SEQ ID | Constitutive | Vacuolar | 17023 |
| | NO: 481) (CBH2) | (CYLCV) | | |
| tobacco | SEQ ID NO: 358 (encoded by SEQ ID | Constitutive | Plastid | 17034 |
| | NO: 481) (CBH2) | (CYLCV) | | |
| tobacco | SEQ ID NO: 168 (encoded by SEQ ID | Constitutive | Vacuolar | 17025 |
| | NO: 482) (Endoglucanase) | (CYLCV) | | |
| tobacco | SEQ ID NO: 168 (encoded by SEQ ID | Constitutive | ER | 17029 |
| | NO: 482) (Endoglucanase) | (CYLCV) | | |
| tobacco | SEQ ID NO: 168 (encoded by SEQ ID | Constitutive | Plastid | 17043 |
| | NO: 482) (Endoglucanase) | (CYLCV) | | |
| maize | SEQ ID NO: 360 (encoded by SEQ ID | Leaf (PepC) | Vacuolar | 15942 |
| | NO: 483) (CBH1) | | | |
| maize | SEQ ID NO: 360 (encoded by SEQ ID | Leaf (PepC) | ER | 15944 |
| | NO: 483) (CBH1) | | | |
| maize | SEQ ID NO: 360 (encoded by SEQ ID | Leaf (PepC) | Plastid | 17026 |
| | NO: 483) (CBH1) | | | |
| maize | SEQ ID NO: 358 (encoded by SEQ ID | Leaf (PepC) | ER | 17013 |
| | NO: 484) (CBH2) | | | |
| maize | SEQ ID NO: 358 (encoded by SEQ ID | Leaf (PepC) | Vacuolar | 17014 |
| | NO: 484) (CBH2) | | | |
| maize | SEQ ID NO: 358 (encoded by SEQ ID | Leaf (PepC) | Plastid | 17042 |
| | NO: 484) (CBH2) | | | |
| maize | SEQ ID NO: 168 (encoded by SEQ ID | Leaf (PepC) | ER | 17084 |
| | NO: 485) (Endoglucanase) | | | |
| maize | SEQ ID NO: 168 (encoded by SEQ ID | Leaf (PepC) | Plastid | 17085 |
| | NO: 485) (Endoglucanase) | | | |
| maize | SEQ ID NO: 168 (encoded by SEQ ID | Leaf (PepC) | Vacuolar | 17086 |
| | NO: 485) (Endoglucanase) | | | |
| maize | SEQ ID NO: 360 (encoded by SEQ ID | Seed (rice glutelin) | ER | 15943 |
| | NO: 483) (CBH1) | | | |
| maize | SEQ ID NO: 34 (encoded by SEQ ID | Seed (rice glutelin) | ER | 17021 |
| | NO: 486) (CBH1) | | | |
| maize | SEQ ID NO: 358 (encoded by SEQ ID | Seed (rice glutelin) | ER | 17012 |
| | NO: 484) (CBH2) | | | |
| maize | SEQ ID NO: 168 (encoded by SEQ ID | Seed (rice glutelin) | ER | 17027 |
| | NO: 485) (Endoglucanase) | | | |
| soybean | SEQ ID NO: 360 (encoded by SEQ ID | Seed (rice glutelin) | PSV | 15928 |
| | NO: 480) (CBH1) | | | |
| soybean | SEQ ID NO: 360 (encoded by SEQ ID | Seed (rice glutelin) | ER | 15929 |
| | NO: 480) (CBH1) | | | |
| soybean | SEQ ID NO: 34 (encoded by SEQ ID | Seed (rice glutelin) | PSV | 15973 |
| | NO: 487) (CBH1) | | | |
| soybean | SEQ ID NO: 34 (encoded by SEQ ID | Seed (rice glutelin) | ER | 15983 |
| | NO: 487) (CBH1) | | | |
| soybean | SEQ ID NO: 358 (encoded by SEQ ID | Seed (rice glutelin) | PSV | 15975 |
| | NO: 481) (CBH2) | | | |
| soybean | SEQ ID NO: 358 (encoded by SEQ ID | Seed (rice glutelin) | ER | 15982 |
| | NO: 481) (CBH2) | | | |
| soybean | SEQ ID NO: 168 (encoded by SEQ ID | Seed (rice glutelin) | PSV | 17050 |
| | NO: 482) (Endoglucanase) | | | |
| soybean | SEQ ID NO: 168 (encoded by SEQ ID | Seed (rice glutelin) | ER | 15984 |
| | NO: 482) (Endoglucanase) | | | |

### Example 10

### Characterizing Enzymes of the Invention

In one embodiment, the invention provides polypeptides, e.g., enzymes, having beta-glycosidase activity, which can be used alone or in combinations, e.g., as “cocktails” or mixtures, in any variety of industrial applications, e.g., for biomass conversion, e.g., for biofuel production. This example characterizes selected properties of some exemplary polypeptides (e.g., enzymes) of this invention.

Substrate Specificity and Enzyme Activity Characterization

| | | | | | | Max if | |
|---|---|---|---|---|---|---|---|
| | | | | | | tot. | |
| | | | | | Max if tot. | conv. = | |
| SEQ ID | | | | | conv. = 200 | 300 | Max if tot. conv. = 500 |
| NO: | Activity | Family | Host strain | Vector | X2 | X3 | X5 |
| 551, 552 | β-glucosidase | GH1, 5 | XL1Blue-MR | pSE420-C′His | 94.2 | 94.7 | 112.8 |
| 625, 626 | Xylosidase | GH3, 3′ | XL1Blue-MR | pSE420-C′His | 117.3 | 152.6 | 364.0 |
| 547, 548 | B-glucosidase | GH1 | XL1Blue-MR | pSE420-C′His | 106.8 | 89.9 | 86.4 |
| 569, 570 | B-glucosidase | GH1 | GAL631 | pSE420-C′His | 100.4 | 84.9 | 94.0 |
| 681, 682 | arabinofuranosidase | GH3 | XL1Blue-MR | pSE420-C′His | 190.8 | 268.7 | 441.1 |
| 581, 582 | β-glucosidase | GH3 | GAL631 | pSE420-C′His | 91.1 | 85.9 | 97.1 |
| 669, 670 | Xylosidase | GH3, 3′ | XL1Blue-MR | pSE420-C′His | 92.3 | 82.3 | 163.1 |
| 563, 564 | B-glucosidase | GH1 | XL1Blue-MR | pSE420-C′His | 98.2 | 94.7 | 121.9 |
| 539, 540 | B-glucosidase | GH1 | XL1Blue-MR | pSE420-C′His | 79.2 | 91.2 | 111.3 |
| 561, 562 | B-glucosidase | GH1 | XL1Blue-MR | pSE420-C′His | 90.0 | 85.1 | 105.6 |
| 565, 566 | B-glucosidase | GH1 | XL1Blue-MR | pSE420-C′His | 85.6 | 87.9 | 101.4 |
| 525, 526 | β-glucosidase | GH3 | XL1Blue-MR | pSE420-C′His | 110.1 | 84.3 | 89.8 |
| 531, 532 | B-glucosidase | GH1 | XL1Blue-MR | pSE420-C′His | 123.2 | 117.1 | 78.1 |
| 645, 646 | β-glucosidase/Xylosidase | GH1 | XL1Blue-MR | pSE420-C′His | 93.2 | 95.4 | 122.3 |
| 423, 424 | β-glucosidase | GH1 | GAL631 | pSE420-C′His | 90.6 | 100.8 | 101.4 |
| 549, 550 | β-glucosidase | GH1 | XL1Blue-MR | pSE420-C′His | 143.0 | 89.6 | 138.0 |
| 529, 530 | B-glucosidase | GH1 | XL1Blue-MR | pSE420-C′His | 99.9 | 81.5 | 79.5 |
| 571, 572 | β-glucosidase | GH1 | GAL631 | pSE420-C′His | 99.4 | 92.5 | 114.0 |
| 573, 574 | β-glucosidase | GH3 | GAL631 | pSE420-C′His | 105.5 | 90.8 | 107.4 |
| 541, 542 | B-glucosidase | GH1 | XL1Blue-MR | pSE420-C′His | 106.1 | 88.3 | 83.6 |
| 543, 544 | B-glucosidase | GH1 | XL1Blue-MR | pSE420-C′His | 103.9 | 81.2 | 80.7 |
| 553, 554 | B-glucosidase | GH1 | XL1Blue-MR | pSE420-C′His | 100.4 | 92.2 | 128.7 |
| 559, 560 | β-glucosidase | GH1 | XL1Blue-MR | pSE420-C′His | 97.1 | 80.4 | 86.0 |
| 595, 596 | β-glucosidase | GH3 | XL1Blue-MR | pSE420-C′His | 146.8 | 217.9 | 303.4 |
| 533, 534 | B-glucosidase | GH1 | GAL631 | pSE420-C′His | 133.7 | 27.6 | 126.1 |
| 575, 576 | B-glucosidase | GH1 | GAL631 | pSE420-C′His | 98.2 | 86.3 | 152.9 |
| 535, 536 | B-glucosidase | GH1 | M15pREP5 | pQET | 46.5 | 64.8 | 56.2 |
| 587, 588 | B-glucosidase | GH3 | GAL631 | pSE420-C′His | 104.0 | 98.0 | 112.0 |
| 583, 584 | B-glucosidase | GH3 | GAL632 | pSE420-C′His | 138.2 | 113.5 | 117.3 |
| 621, 622 | Xylosidase | GH3, 3′ | XL1Blue-MR | pSE420-C′His | 93.8 | 153.5 | 320.2 |
| 631, 632 | Oligomerase/Xylosidase | GH3 | P. pastorisx33 | pPICZAlpha | 90.1 | 88.6 | 243.5 |
| 589, 590 | β-glucosidase | GH1 | XL1Blue-MR | pSE420-C′His | 89.1 | 92.6 | 134.8 |
| 591, 592 | β-glucosidase | GH1 | GAL631 | pSE420-C′His | 113.0 | 104.7 | 142.4/109.3 |
| 527, 528 | B-glucosidase | GH3 | GAL631 | pSE420-C′His | 111.0 | 116.2 | 152.7 |
| 537, 538 | β-glucosidase | GH3 | M15pREP4 | pQET | 80.3 | 83.8 | 76.2 |
| 555, 556 | β-glucosidase | GH1 | M15pREP4 | pQET | 90.3 | 78.8 | 65.2 |
| 699, 700 | Xylosidase | GH52 | XL1Blue-MR | pSE420-C′His | 175.2 | 214.0 | 415.4 |

| SEQ ID | | Ara-Xyl | | | | | |
|---|---|---|---|---|---|---|---|
| NO: | Activity | hydrolysis | C2 | C5 | pNP-BD-Gluc | pNP-BD-Xyl | pNP-a-Ara |
| 551, 552 | β-glucosidase | no | 118.0 | 98.4 | 28.3 | 38.7 | 37.2 |
| 625, 626 | Xylosidase | no | 136.7 | 148.3 | 878.1 | 460.4 | 814.7 |
| 547, 548 | B-glucosidase | no | 109.2 | 233.0 | 153.9 | in anal | 33.8 |
| 569, 570 | B-glucosidase | no | 86.5 | 102.0 | 0.0 | 14.8 | 38.4 |
| 681, 682 | arabinofuranosidase | yes | 93.6 | 114.1 | 0.9 | 2.6 | 10.9 |
| 581, 582 | β-glucosidase | no | 87.1 | 115.1 | 3.6 | 16.7 | 38.5 |
| 669, 670 | Xylosidase | no | 105.4 | 97.2 | −0.6 | 34.2 | 22.4 |
| 563, 564 | B-glucosidase | no | 115.7 | 110.1 | 425.3 | 41.2 | 35.7 |
| 539, 540 | B-glucosidase | no | 108.6 | 116.8 | 642.0 | 62.0 | 35.8 |
| 561, 562 | B-glucosidase | yes | 138.9 | in anal | in anal | in anal | in anal |
| 565, 566 | B-glucosidase | no | 114 | 141.1 | 129.6 | 13.9 | 35.6 |
| 525, 526 | β-glucosidase | no | 82.1 | 174.4 | 31.9 | 14.0 | 37.3 |
| 531, 532 | B-glucosidase | no | 89.0 | 227.7 | 311.6 | 19.5 | 37.7 |
| 645, 646 | β-glucosidase/Xylosidase | no | 131.4 | 105.1 | −0.6 | 38.5 | 36.1 |
| 423, 424 | β-glucosidase | no | 174.7 | 248.8 | 665.7 | 17.4 | 39.7 |
| 549, 550 | β-glucosidase | no | 139.8 | 136.3 | 311.2 | 42.0 | 37.1 |
| 529, 530 | B-glucosidase | no | 79.7 | 97.0 | 53.7 | 4.0 | 34.2 |
| 571, 572 | β-glucosidase | no | 145.5 | 134.0 | 253.9 | 23.2 | 42.7 |
| 573, 574 | β-glucosidase | no | 96.5 | 88.7 | 0.0 | 12.8 | 37.6 |
| 541, 542 | B-glucosidase | no | 111.0 | 219.2 | 97.7 | 4.3 | 34.0 |
| 543, 544 | B-glucosidase | no | 88.2 | 93.5 | 43.1 | 5.3 | 34.3 |
| 553, 554 | B-glucosidase | no | 133.5 | 100.9 | 234.4 | 39.0 | 36.7 |
| 559, 560 | β-glucosidase | no | 122.3 | 233.5 | 554.7 | 29.6 | 39.0 |
| 595, 596 | β-glucosidase | no | 97.5 | 90.9 | 0.0 | 13.4 | 38.0 |
| 533, 534 | B-glucosidase | no | 122.6 | 101.3 | 0.0 | 13.1 | 37.9 |
| 575, 576 | B-glucosidase | yes | 149.2 | 257.9 | 1006.0 | 257.1 | 39.4 |
| 535, 536 | B-glucosidase | no | 88.8 | 70.5 | 5.8 | 11.8 | 42.9 |
| 587, 588 | B-glucosidase | no | 127.1 | 268.6 | 977.9 | 104.7 | 60.4 |
| 583, 584 | B-glucosidase | no | 141.2 | 170.0 | 11.8 | 13.6 | 38.9 |
| 621, 622 | Xylosidase | no | 135.9 | 100.3 | 2.3 | 38.2 | 23.6 |
| 631, 632 | Oligomerase/Xylosidase | no | 68.6 | 101.5 | 13.4 | 312.7 | 50.7 |
| 589, 590 | β-glucosidase | no | 100.0 | 103.3 | 14.8 | 36.4 | 22.4 |
| 591, 592 | β-glucosidase | yes? | 138.9* | 302.9 | 1010.8 | 51.7 | 38.5 |
| 527, 528 | B-glucosidase | yes? | 150.6 | 223.7 | 158.9 | 16.0 | 39.0 |
| 537, 538 | β-glucosidase | no | 90.7 | 87.6 | 99.9 | 11.8 | 2.6 |
| 555, 556 | β-glucosidase | no | 124.8 | 116.6 | 803.0 | 89.5 | 40.0 |
| 699, 700 | Xylosidase | yes | 138.2 | 106.2 | 0.1 | 167.9 | 38.5 |

| SEQ ID | | | | | | | |
|---|---|---|---|---|---|---|---|
| NO: | Activity | Family | Host strain | Vector | temp opt | pH opt | S.A. |
| 531, 532 | B-glucosidase | GH1 | XL1Blue-MR | pSE420-C′His | 37 | 6 | 8.02 |
| 645, 646 | β- | GH1 | XL1Blue-MR | pSE420-C′His | 37 | 5 | 14.94 |
| | glucosidase/Xylosidase | | | | | | |
| 541, 542 | B-glucosidase | GH1 | XL1Blue-MR | pSE420-C′His | 37 | 6 | 0.43 |
| 595, 596 | β-glucosidase | GH3 | XL1Blue-MR | pSE420-C′His | 37 | 5 | 0.489 |
| 591, 592 | β-glucosidase | GH1 | GAL631 | pSE420-C′His | 37 | 6 | 2.699 |
| 431, 432 | Glycosidase | GH1, 5 | XL1Blue-MR | pSE420-C′His | 60 | 6 | 0.7 |
| 547, 548 | B-glucosidase | GH1 | XL1Blue-MR | pSE420-C′His | 60 | 6 | 3.5 |
| 539, 540 | B-glucosidase | GH1 | XL1Blue-MR | pSE420-C′His | 60 | 7 | 10.86 |
| 545, 546 | B-glucosidase | GH1 | XL1Blue-MR | pSE420-C′His | 60 | 5 | 3.83 |
| 565, 566 | B-glucosidase | GH1 | XL1Blue-MR | pSE420-C′His | 60 | 6 | 0.89 |
| 525, 526 | β-glucosidase | GH3 | XL1Blue-MR | pSE420-C′His | 60 | 5 | 0.5 |
| 549, 550 | B-glucosidase | GH1 | XL1Blue-MR | pSE420-C′His | 60 | 7 | 4.06 |
| 529, 530 | B-glucosidase | GH1 | XL1Blue-MR | pSE420-C′His | 60 | 6 | 2.75 |
| 543, 544 | B-glucosidase | GH1 | XL1Blue-MR | pSE420-C′His | 60 | 6 | 0.264 |
| 575, 576 | B-glucosidase | GH1 | GAL631 | pSE420-C′His | 60 | 6 | 164 |
| 589, 590 | β-glucosidase | GH1 | XL1Blue-MR | pSE420-C′His | 60 | 5 | 0.31 |
| 537, 538 | β-glucosidase | GH3 | M15pREP4 | pQET | 60 | 5 | 2.25 |
| 563, 564 | B-glucosidase | GH1 | XL1Blue-MR | pSE420-C′His | 80 | 6 | 13.5 |
| 553, 554 | B-glucosidase | GH1 | XL1Blue-MR | pSE420-C′His | 80 | 6 | 3 |
| 559, 560 | β-glucosidase | GH1 | XL1Blue-MR | pSE420-C′His | 80 | 6 | 4.26 |
| 569, 570 | B-glucosidase | GH1 | GAL631 | pSE420-C′His | | | |
| 581, 582 | β-glucosidase | GH3 | GAL631 | pSE420-C′His | | 5 | |
| 423, 424 | β-glucosidase | GH1 | GAL631 | pSE420-C′His | | 6 | |
| 571, 572 | β-glucosidase | GH1 | GAL631 | pSE420-C′His | | 7 | |
| 577, 578 | β-glucosidase | GH3 | GAL631 | pSE420-C′His | | 5 | |
| 573, 574 | β-glucosidase | GH3 | GAL631 | pSE420-C′His | | | |
| 533, 534 | B-glucosidase | GH1 | GAL631 | pSE420-C′His | | | |
| 535, 536 | B-glucosidase | GH1 | M15pREP5 | pQET | | | |
| 587, 588 | B-glucosidase | GH3 | GAL631 | pSE420-C′His | | | |
| 557, 558 | B-glucosidase | GH3 | P. pastorisx33 | pPICZAlpha | | | |
| 527, 528 | B-glucosidase | GH3 | GAL631 | pSE420-C′His | | 7 | |

| | | | Activity | | | |
|---|---|---|---|---|---|---|
| | | Specific Activity, | on | Activity on | | |
| SEQ ID | | 37 C. (pNP-B- | C2? | Cellopentose? | | |
| NO: | Activity | Glucopyranoside) | (37 C.) | (37 C.) | Active at 55 C.? | Active pH Range (55 C.) |
| 531, 532 | B-glucosidase | 311.6 | Y | Y | Y | 7, 9 |
| 645, 646 | β- | | | | | |
| | glucosidase/Xylosidase | | | | | |
| 541, 542 | B-glucosidase | 97.7 | Y | Y | Y | 5, 7, 9 |
| 595, 596 | β-glucosidase | 0.0 | | | | |
| 591, 592 | β-glucosidase | 1010.8 | Y | Y | Y | 5, 7, 9 |
| 431, 432 | Glycosidase | | | | | |
| 547, 548 | B-glucosidase | 153.9 | Y | Y | Y | 5, 7, 9 |
| 539, 540 | B-glucosidase | | | | | |
| 545, 546 | B-glucosidase | | | | | |
| 565, 566 | B-glucosidase | 129.6 | Y | Y | Y | 5, 7, 9 |
| 525, 526 | β-glucosidase | 31.9 | Y | Y | | |
| 549, 550 | B-glucosidase | | | | | |
| 529, 530 | B-glucosidase | 53.7 | | | | |
| 543, 544 | B-glucosidase | 43.1 | | | | |
| 575, 576 | B-glucosidase | 1006.0 | Y | Y | Y | 5, 7, 9 |
| 589, 590 | β-glucosidase | | | | | |
| 537, 538 | β-glucosidase | 99.9 | | | | |
| 563, 564 | B-glucosidase | 172.4 | | | | |
| 553, 554 | B-glucosidase | | | | | |
| 559, 560 | β-glucosidase | 554.7 | Y | Y | Y | 5, 7, 9 |
| 569, 570 | B-glucosidase | 0.0 | | | | |
| 581, 582 | β-glucosidase | 3.6 | | | | |
| 423, 424 | β-glucosidase | 665.7 | Y | Y | N | |
| 571, 572 | β-glucosidase | 253.9 | Y | Y | | |
| 577, 578 | β-glucosidase | | | | | |
| 573, 574 | β-glucosidase | 0.0 | | | | |
| 533, 534 | B-glucosidase | 0.0 | | | | |
| 535, 536 | B-glucosidase | 5.8 | | | | |
| 587, 588 | B-glucosidase | 977.9 | | | Y | |
| 557, 558 | B-glucosidase | | | | Y | 5, 7, 9 |
| 527, 528 | B-glucosidase | 158.9 | Y | Y | N | |

| | | Product Inhibition (1 = Inhibition at Low Glucose Doses, 2 = | | |
|---|---|---|---|---|
| | pH Optima | Inhibition at Higher Glucose Doses, 3 = Minimal Inhibition at | | |
| SEQ ID NO: | 55 C. | High Glucose Doses) | active at ≧ 50 C. | active at ≧ 60 C. |
| 531, 532 | 7 | | | |
| 645, 646 | | | | |
| 541, 542 | 7 | | Y | Y |
| 595, 596 | | | | |
| 591, 592 | 5, 7 | 2 | Y | Y |
| 431, 432 | | | | |
| 547, 548 | 5 | | Y | Y |
| 539, 540 | | | Y | Y |
| 545, 546 | | | Y | Y |
| 565, 566 | 5 | | Y | Y |
| 525, 526 | | | Y | Y |
| 549, 550 | | | Y | |
| 529, 530 | | | Y | |
| 543, 544 | | | | |
| 575, 576 | 5, 7 | 3 | | |
| 589, 590 | | | | |
| 537, 538 | | | | |
| 563, 564 | | | Y | Y |
| 553, 554 | | | Y | Y |
| 559, 560 | 7 | 2 | Y | Y |
| 569, 570 | | | | |
| 581, 582 | | | | |
| 423, 424 | | | | |
| 571, 572 | | | | |
| 577, 578 | | | Y | Y |
| 573, 574 | | | | |
| 533, 534 | | | | |
| 535, 536 | | | | |
| 587, 588 | | | Y | Y |
| 557, 558 | | 1 | Y | Y |
| 527, 528 | | | | |

| | | | | | | | | % residual | | |
|---|---|---|---|---|---|---|---|---|---|---|
| | | | | | | | % residual | activity | % residual | |
| | | | | | Activity at | Activity at | activity | after | activity after | Activity |
| SEQ ID | | | Host | | 37-90 C. at | 37-90 C. at | after 0-4 h | 0-4 h at | 0-4 h at | at 50 C. |
| NO: | Activity | Family | strain | Vector | pH 5** | pH 7** | at 60 C.*** | 70 C.*** | 80 C.*** | pH5**** |
| 531, 532 | B-glucosidase | GH1 | XL1Blue- | pSE420- | 37-40 (10% | 37-40 (10% | 0 after 1 h | 0 after 1 h | 0 after 1 h | |
| | | | MR | C′His | at 50 C.) | at 50 C.) | | | | |
| 549, 550 | B-glucosidase | GH1 | XL1Blue- | pSE420- | 37-40 (20% | 37-50 | NT | NT | NT | 227 mM |
| | | | MR | C′His | at 50 C.) | | | | | glucose |
| 561, 562 | B-glucosidase | GH1 | XL1Blue- | pSE420- | 37-40 (60% | 37-40 (60% | 0 | 0 | 0 | |
| | | | MR | C′His | at 50 C.) | at 50 C.) | | | | |
| 525, 526 | β-glucosidase | GH3 | XL1Blue- | pSE420- | 37-50 | 37-50 | NT | 0 after 1 h | 0 after 1 h | 457 mM |
| | | | MR | C′His | | | | | | glucose |
| 545, 546 | B-glucosidase | GH1 | XL1Blue- | pSE420- | 37-50 (20% | 37-50 (20% | 0 after 1 h | 0 after 1 h | 0 after 1 h | 520 mM |
| | | | MR | C′His | at 60 C.) | at 60 C.) | | | | glucose |
| 577, 578 | β-glucosidase | GH3 | GAL631 | pSE420- | 37-50 (50% | 37-60 | 0 | 0 | 0 | 440 mM |
| | | | | C′His | at 60 C.) | (weak) | | | | glucose |
| 553, 554 | B-glucosidase | GH1 | XL1Blue- | pSE420- | 37-60 | 37-60 | 50% after 1 h, | 0 after 1 h | 0 after 1 h | 433 mM |
| | | | MR | C′His | | | 25% after 2 h, | | | glucose |
| | | | | | | | 0% after | | | |
| | | | | | | | 3 h (all | | | |
| | | | | | | | C); 7% after | | | |
| | | | | | | | 0.5 h at GP | | | |
| 529, 530 | B-glucosidase | GH1 | XL1Blue- | pSE420- | 37-60 | 37-60 | 90% after 4 h | 0 | 0 | 345 mM |
| | | | MR | C′His | | | (C); 80% after | | | glucose |
| | | | | | | | 4 h (GP) | | | |
| 557, 558 | B-glucosidase | GH3 | P. | pPICZAlpha | 37-60 | 37-50 | 10% after 0.5 h | 0 | 0 | 338 mM |
| | | | pastorisx33 | | (<20% at | | at 60 C., 0 after | | | glucose |
| | | | | | 70 C.) | | 1 h (both C. | | | |
| | | | | | | | and GP) | | | |
| 541, 542 | B-glucosidase | GH1 | XL1Blue- | pSE420- | 37-60 (20% | 37-60 (10% | 10% after 1 h, | 0 | 0 | 333 mM |
| | | | MR | C′His | at 70 C.) | at 70 C.) | 5% after 3 h | | | glucose |
| | | | | | | | (C); 0 (GP) | | | |
| 563, 564 | B-glucosidase | GH1 | XL1Blue- | pSE420- | 37-60 (50% | 37-60 (50% | 100% after 4 h | 0 | 0 | |
| | | | MR | C′His | at 70 C.) | at 70 C.) | (C); 70% after | | | |
| | | | | | | | 4 h (GP) | | | |
| 591, 592 | β-glucosidase | GH1 | GAL631 | pSE420- | 37-60 (50% | 37-50 (40% | 0 after 1 h (C, | 0 after 1 h | 0 after 1 h | 174 mM |
| | | | | C′His | at 70 C.) | at 60 C.) | GP) | (C, GP) | (C, GP) | glucose |
| 543, 544 | B-glucosidase | GH1 | XL1Blue- | pSE420- | 37-60 | 37-60 | NT | NT | NT | |
| | | | MR | C′His | (weak at all | (weak at all | | | | |
| | | | | | Ts) | Ts) | | | | |
| 595, 596 | β-glucosidase | GH3 | XL1Blue- | pSE420- | 37-60 | 37-50 | NT | NT | NT | |
| | | | MR | C′His | (weak) | | | | | |
| 547, 548 | B-glucosidase | GH1 | XL1Blue- | pSE420- | 37-70 (25% | 37-60 (60% | 100% (90% | 0 | 0 | 423 mM |
| | | | MR | C′His | at 80 C.) | at 70 C.) | after 4 h) | | | glucose |
| 559, 560 | β-glucosidase | GH1 | XL1Blue- | pSE420- | 37-80 | 37-90 | 90% after 4 h | 90% after | 140% after | 164 mM |
| | | | MR | C′His | | | (C); 70% after | 4 h (C); | 4 h on C, | glucose |
| | | | | | | | 4 h (GP) | 60% after | confirm!20% | |
| | | | | | | | | 4 h (GP) | after 4 h | |
| | | | | | | | | | (GP) | |
| 537, 538 | β-glucosidase | GH3 | M15pREP4 | pQET | 37-80 | NA | NT | NT | NT | |
| | | | | | (weak at all | | | | | |
| | | | | | Ts) | | | | | |
| 587, 588 | B-glucosidase | GH3 | GAL631 | pSE420- | 37-90 | 37-40 | 25% after | 0 | 0 | 260 mM |
| | | | | C′His | | | 0.5 h, 0 after | | | glucose |
| | | | | | | | 3 h(C); 0 after | | | |
| | | | | | | | 1 h (GP) | | | |

| SEQ ID | | Product inhibition | % purity of | | |
|---|---|---|---|---|---|
| NO: | Activity | (mM glucose) | prep | Activity at 37 C. pH5**** | Activity at 50 C. pH5**** |
| 531, 532 | B-glucos idase | NT | | | |
| 549, 550 | B-glucosidase | NT | 8 | 294 mM glucose; 10 U/ml | 227 mM glucose |
| 561, 562 | B-glucosidase | NT | | | |
| 525, 526 | β-glucosidase | 25 | 5 | 423 mM glucose; 4 U/ml | 457 mM glucose |
| 545, 546 | B-glucosidase | 25 | <3 | 450 mM glucose; 5 U/ml | 520 mM glucose |
| 577, 578 | β-glucosidase | NT | 8 | 232 mM glucose; 2 U/ml | 440 mM glucose |
| 553, 554 | B-glucosidase | 100 (starts at 50) | 11 | 362 mM glucose; 2 U/ml | 433 mM glucose |
| 529, 530 | B-glucosidase | 200 (starts at 50) | 8 | 277 mM glucose; 6 U/ml | 345 mM glucose |
| 557, 558 | B-glucosidase | 50 (starts at 25) | 35 | 488 mM glucose; 10 U/ml | 338 mM glucose |
| 541, 542 | B-glucosidase | 200 (starts at 25) | <3 | 412 mM glucose; 2 U/ml | 333 mM glucose |
| 563, 564 | B-glucosidase | >200, starts at 25 | | | |
| 591, 592 | β-glucosidase | 200 (starts at 25) | 7 | 158 mM glucose; 24 U/ml | 174 mM glucose |
| 543, 544 | B-glucosidase | NT | | | |
| 595, 596 | β-glucosidase | NT | | | |
| 547, 548 | B-glucosidase | 400 (starts at 100) | <5 | 461 mM glucose; 3 U/ml | 423 mM glucose |
| 559, 560 | β-glucosidase | 100 (starts at 25); | <3 | 242 mM glucose; 8 U/ml | 164 mM glucose |
| | | confirm with new | | | |
| | | batch | | | |
| 537, 538 | β-glucosidase | NT | | | |
| 587, 588 | B-glucosidase | 100 (starts at 25) | 8 | 273 mM glucose; 13 U/ml | 260 mM glucose |

*All enzymes tested at pH 5 and pH 7, 10 mM cellobiose, 30 min at 37° C., 40° C., 50° C., 60° C., 70° C., 80° C. and 90° C.
**10 mM celobiose substrate
***C = cellobiose; GP = 4MU-GP substrate
****Endpoint, 30 min reaction, 10 mM cellobiose pH 5; 0.5 mg SPEED prep, 0.4 mg A. niger beta-glucosidase (b-gluc); mM glucose released; U/ml on fluorescent substarte (4MU-GP)
Cellobiose digest 30 min at pH5/60 C.; 4MU-GP digest 20 min at pH5/RT

### Example 11

### Protein Analysis of Transgenic Plants

The invention provides transgenic plants (and cells and seeds and plant parts derived from those transgenic plants) comprising expression systems of this invention comprising vectors, recombinant viruses, artificial chromosomes, etc. of this invention, and/or comprising nucleic acids of this invention, including nucleic acids encoding enzymes of this invention, and including sequences complementary to the enzyme-encoding sequences; and this example describes making some of these embodiments.

Protein extracts were obtained from approximately 100 mg of leaf tissue or flour generated from maize and soybean seed from non-transgenic and transgenic plants. Leaf material was placed into 96 deep well blocks containing small steel balls and pre-cooled on dry ice. Samples were ground to a fine powder using a GENO/GRINDER™ (SPEC/CERTIPREP™, Metuchen, N.J.). Flour samples were prepared by pooling approximately 10-20 seed and grinding to a fine powder using a KLECO™ Grinder (Gracia Machine Company, Visalia, Calif.). Samples were extracted in 250-500 μl of either Western Extraction Buffer (WEB=12.5 mM sodium borate, pH10; 2% BME; and 1% SDS) or assay buffer at room temperature for approximately 30 minutes followed by centrifugation for 5 minutes at 13,000 rpm.

SDS-polyacrylamide gel electrophoresis (SDS-PAGE) was performed by transferring 100 μl of WEB samples to an Eppendorf tube and add 25 μl 4× BioRad LDS or modified BIORAD™ (Hercules, Calif.) loading buffer (4× BioRad LDS:BME at a ratio of 2:1). Heat samples for 10 minutes at 70° C. then immediately place on ice for 5 minutes. Spin samples briefly, and transfer back on to ice. Sample extracts (5-10 μl) were run on BioRad 4-12% Bis/Tris protein gel (18 well) using MOPS buffer.

Immunoblot analysis was performed by transferring SDS-PAGE gels onto a nitrocellulose membrane using chilled NUPAGE™ transfer buffer (Invitrogen) for 30 minutes at 100 volts. Total protein transferred to the blot was visualized using Ponceau stain (Sigma). Following Ponceau staining, the membrane was incubated in blocking buffer for 30 minutes in TBST wash buffer (30 mM Tris-HCL, pH 7.5, 100 mM NaCl, and 0.05% Tween 20) with 3% dry milk, then washed three times for 5 minutes in TBST. Polyclonal goat or rabbit primary antibody was added at 1 ug/ml in TBST wash buffer with 3% milk, and the blot incubated 2 hours to overnight. Following overnight incubation, the blot was washed three times for 5 minutes each in TBST wash buffer. Secondary antibody (Rabbit-AP or Goat-AP) was diluted 1:8000 (in TBST) and added to blot for 30 minutes. Following incubation in the secondary antibody, the blot was again washed three times for 5 minutes each. Visualization of immuno reactive bands was carried out by adding Moss BCIP/NBT—alkaline phosphatase substrate. Blots were rinsed thoroughly in water following incubation in the BCIP/NBT substrate and allowed to air dry.

Western blots analysis of sample extracts used for activity analysis showed a correlation between accumulation of an immuno-reactive protein and enzyme activity (described in Example 12). CBH1 with ER targeting sequence (construct 15936) was detected as a band that migrates close to the predicted size of the full length enzyme (56.6 kD). A second, smaller band of about 51 kD was also detected in the western blot. CBH1 targeted to the leaf vacuole (construct 15935) accumulated predominantly as a 51 kD protein. Western blot data for transgenic plants generated with constructs 15935 and 15936 are summarized in table 8, below.

Western blot analysis was used to screen transgenic maize plants generated with construct 15942 and construct 15944. The maize leaf expressed SEQ ID NO:360 CBH1 with ER targeting sequence (construct 15944) was detected as a band that migrates close to the predicted size of the full length enzyme (57 kD). A second, broad band centered around 51 kD was also detected. Vacuolar targeted SEQ ID NO:360 CBH1 (construct 15942) shows a broad band at approximately 51 kD with a minor band at 57 kD. Western blot data is summarized in table 9, below, for construct 15942 and table 10, below for construct 15944.

### Example 12

### Enzyme Extraction and Activity Analysis of Transgenic Events

In one embodiment, isolated or recombinant enzymes or other polypeptides of the invention are harvested from transgenic plants, cells, seeds and/or plant parts of this invention; and this example describes an exemplary embodiment.

Approximately 100 mg of fresh leaf tissue or seed flour of a transgenic plant was extracted in 5 to 10 ml of one of the following buffers: (A) 100 mM Na acetate, 0.02% Tween, 0.02% Na azide pH 4.75, 1% PVP and COMPLETE™ protease inhibitor cocktail tablets (Roche); (B) 100 mM Na acetate, 1 mg/ml BSA, 0.02% Tween, and 0.02% Na azide pH 4.75; or (C) 100 mM Sodium Acetate pH 5.3, 100 mM NaCl, 1 mg/ml Gelatin, 1 mM EDTA, 0.02% TWEEN-20™, 0.02% NaN₃. Alternative buffers for extracting protein from leaf or from seed are well known in the art. Samples were placed on benchtop rotators for 30-60 minutes then centrifuged at 3000 rpm for 10 minutes. For fresh leaf samples, the amount of total protein extracted was measured by Pierce BCA protocol as outlined in product literature. Cellulase activity assays were carried out using one of the following substrates: pNP-lactoside, methylumbelliferyl-lactoside (MUL), carboxymethyl-cellulose, oat-β glucan, phosphoric acid treated cellulose (PASC), Avicel, or other commercially available substrates used for measuring cellulase activity following previously published protocols (see, e.g., Methods in Enzymology, Vol 160). Enzyme activity data generated for transgenic plants is outlined in tables 8 through 10, below.

TABLE 8
Summary of cellobiohydrolase I (CBHI) activity in transgenic
tobacco events expressing dicot optimized SEQ ID NO: 360
targeted to the vacuole (construct 15935) and ER
(construct 15936) of tobacco leaves. Samples were extracted
in buffer A and CBH1 activity was assayed on
methylumbelliferyl-lactoside as the substrate.

| Construct | Plant ID | nmol/min/mg | | AVICEL ™ |
|---|---|---|---|---|
| number | number | protein | Western blot | binding assay |
| 15935 | Nt22-1A | 0.466 | + | ND |
| 15935 | Nt22-6B | 0.519 | + | ND |
| 15935 | Nt22-7A | 0.685 | + | ND |
| 15935 | Nt22-10A | 0.587 | + | ND |
| 15935 | Nt22-11A | 0.500 | + | ND |
| 15935 | Nt22-15A | 0.363 | + | ND |
| 15935 | Nt22-16A | 1.337 | + | + |
| 15935 | Nt22-17A | 0.650 | + | ND |
| 15935 | Nt22-18A | 1.079 | + | ND |
| 15935 | Nt22-19A | 0.009 | − | − |
| 15935 | Nt22-23B | 1.811 | + | + |
| 15935 | Nt22-24B | 1.151 | + | ND |
| 15935 | Nt22-30B | 1.338 | + | ND |
| 15936 | Nt23-2B | 0.170 | + | ND |
| 15936 | Nt23-5A | 0.118 | + | ND |
| 15936 | Nt23-9A | 0.670 | + | ND |
| 15936 | Nt23-11A | 0.666 | + | + |
| 15936 | Nt23-12A | 0.410 | + | ND |
| 15936 | Nt23-16A | 0.354 | + | ND |
| 15936 | Nt23-17B | 0.597 | + | ND |
| 15936 | Nt23-22A | 0.484 | + | ND |
| 15936 | Nt23-23B | 0.907 | + | + |
| 15936 | Nt23-24B | 0.162 | + | ND |
| 15936 | Nt23-26B | 0.203 | + | ND |
| 15936 | Nt23-29B | 0.626 | + | ND |
| 15936 | Nt23-30B | 0.082 | − | − |
| 15936 | Nt23-32B | 0.190 | + | ND |
| Non- | Non- | 0.007 | − | ND |
| transgenic | transgenic | | | |
| control | control | | | |
| Non- | Non- | −0.010 | − | ND |
| transgenic | transgenic | | | |
| control | control | | | |

ND = not determined

TABLE 9
Summary of cellobiohydrolase I (CBHI) activity in transgenic maize
events (construct 15942) expressing monocot optimized SEQ
ID NO: 360 targeted to the vacuole of maize leaves. Samples
were extracted in buffer A and CBH1 activity was assayed on
methylumbelliferyl-lactoside as the substrate.

| | Avg nmol/min/mg | Standard | |
|---|---|---|---|
| Plant ID Number | Protein | Deviation | Western Blot |
| 001A | 1.51 | 0.46 | + |
| 002A | 0.63 | 0.05 | + |
| 003A | 0.53 | 0.18 | + |
| 004A | 1.01 | 0.34 | + |
| 005A | 0.04 | 0.01 | − |
| 006A | 0.03 | 0.01 | − |
| 007A | 2.34 | 0.48 | + |
| 008A | 0.48 | 0.05 | + |
| 009A | 0.65 | 0.05 | + |
| 011A | 0.11 | 0.05 | − |
| 012A | 1.47 | 0.12 | + |
| 013A | 1.88 | 0.62 | + |
| 014A | 0.68 | 0.14 | + |
| 015A | 3.45 | 0.17 | + |
| 016A | 3.17 | 0.42 | + |
| 018A | 2.32 | 0.52 | + |
| 019A | 4.33 | 2.02 | + |
| 021A | 0.88 | 0.01 | + |
| 022A | 2.69 | 0.15 | + |
| 023A | 0.03 | 0.00 | − |
| 024A | 4.84 | 0.36 | + |
| 025A | 1.77 | 0.22 | + |
| 026A | 0.57 | 0.04 | + |
| 027A | 1.87 | 0.77 | + |
| 028A | 8.43 | 1.09 | + |
| 029A | 1.88 | 0.70 | + |
| 030A | 1.08 | 0.04 | + |
| Nontransgenic | 0.07 | 0.00 | − |
| control | | | |

TABLE 10
Summary of cellobiohydrolase I (CBHI) activity in transgenic maize
events (construct 15944) expressing the monocot optimized,
exemplary SEQ ID NO: 360 of the invention targeted to the ER of
maize leaves. Samples were extracted in buffer A and CBH1 activity
was assayed on methylumbelliferyl-lactoside as the substrate.

| | Avg nmol/min/mg | Standard | |
|---|---|---|---|
| Plant ID Number | protein | Deviation | Western Blot |
| 001A | 1.71 | 0.09 | + |
| 002A | 0.01 | 0.00 | − |
| 003A | 1.10 | 0.13 | + |
| 004A | 0.03 | 0.00 | − |
| 005A | 0.63 | 0.04 | + |
| 006A | ND | ND | − |
| 007A | ND | ND | − |
| 008A | ND | ND | − |
| 009A | 1.20 | 0.04 | + |
| 010A | ND | ND | − |
| 011A | ND | ND | + |
| 012A | 1.34 | 0.09 | + |
| 013A | 5.85 | 0.43 | + |
| 014A | 1.20 | 0.07 | + |
| 015A | 1.95 | 0.19 | + |
| 016A | ND | ND | − |
| 017A | 2.50 | 0.07 | + |
| 018A | ND | ND | + |
| 019A | ND | ND | − |
| 020A | 0.91 | 0.07 | + |
| 021A | 2.34 | 0.05 | + |
| 022A | ND | ND | − |
| 023A | ND | ND | + |
| 024A | ND | ND | − |
| 025A | ND | ND | + |
| 026A | ND | ND | + |
| 027A | 1.51 | 0.09 | + |
| 028A | ND | ND | + |
| 029A | ND | ND | + |
| 030A | ND | ND | + |
| 031A | 2.36 | 0.07 | + |
| 032A | ND | ND | − |
| 033A | 1.59 | 0.11 | + |
| 034A | ND | ND | + |
| 035A | 1.14 | 0.11 | + |
| 036A | 1.06 | 0.09 | + |
| 037A | 1.27 | 0.21 | + |
| 038A | 0.55 | 0.01 | + |
| 039A | 1.51 | 0.02 | + |
| 040A | 1.36 | 0.15 | + |
| 041A | 0.53 | 0.01 | + |
| 042A | 0.02 | 0.00 | − |
| 043A | 1.15 | 0.05 | + |
| 044A | 0.81 | 0.03 | + |
| 045A | ND | ND | − |
| 046A | 0.52 | 0.03 | + |

ND = not determined

### Example 13

### Crystalline Cellulose Binding and Hydrolysis Assays

In one embodiment, isolated, synthetic or recombinant enzymes or other polypeptides of the invention bind to and/or catalyze the hydrolysis of cellulose, e.g., crystalline cellulose, and this example describes an exemplary embodiment and assay—which can be used to determine if a polypeptide has enzymatic, e.g., hydrolase, such as cellulase, activity.

AVICEL™ Microcrystalline Cellulose (MCC) Binding Assay: Approximately 100 mg of leaf tissue was extracted in 5 mL of assay buffer (A), as described above. Following extraction, approximately 250 ul of sample was incubated with 25 mg AVICEL™ MCC for 0 and 60 minutes. Zero time point samples were added to Eppendorf tubes placed on ice prior to addition of extracts and immediately processed. Samples were incubated for 60 minutes on a benchtop vortex at room temperature. After incubation, samples were centrifuged for 5 minutes at 13000 rpm in an Eppendorf centrifuge. Supernatants were carefully removed and the AVICEL™ MCC washed 3× with ice cold water. Following the final wash, 80 ul of western extraction buffer (WEB) and 25 ul of BioRad 4× loading buffer was added the sample. Samples were vortexed then placed at 70 degrees for 10 minutes. The AVICEL™ MCC was pelleted at 13000 rpm and the supernatants removed and analyzed by western blot as described above.

Transgenic plants derived from construct 15935 (Nt22-16A, Nt22-19A) and construct 15936 (Nt23-11A, Nt23-23B, Nt23-30B) were analyzed through the Avicel MCC Binding assay described in the above paragraph. Plants Nt22-16A, Nt23-11A and Nt23-23B were positive by western blot analysis while plants Nt22-19A and Nt23-30B were negative in the AVICEL™ MCC Binding assay. This data is summarized in Table 8, above.

AVICEL™ MCC Hydrolysis assay. Transgenic leaf samples were lyophilized then ground to a find powder using a Kleco grinder. Approximately 150 mg of ground leaf material was weighed out and extracted in 4 ml of buffer A at RT for 30 minutes. Samples were centrifuged and supernatants removed. One ml of each leaf extract, fungal expressed SEQ ID NO:360 or Trichoderma reesei CBH1 (Megazyme International) enzyme, or fungal enzymes added to non-expressing transgenic extract was added to 50 mg of Avicel MCC and samples placed on a vortex at 37 degrees. Protein concentrations were measured using BCA reagent (Pierce). Duplicate 100 μl samples were removed at 0, 24, 48, and 72 hours. Sugar analysis was carried out by HPLC analysis. Data generated for maize transgenic plants transformed with construct 15944 (the exemplary SEQ ID NO:360 CBH1 targeted to the ER) is shown in table 11, below.

Protein extracts from the transgenic plants were equivalent for total protein content; however, the data does not represent the relative level of expression of the exemplary SEQ ID NO:360 in transgenic plants. The data in table 11 demonstrates that plant expressed cellulases are active in the AVICEL™ MCC assay which demonstrates binding of the cellulase to a substrate and subsequent cellulase activity.

TABLE 11
Liberation of cellobiose from AVICEL ™ MCC

| | mg/mL cellobiose | Standard |
|---|---|---|
| Transgenic number | produced at 72 hours | Deviation |
| 013A | 2.644 | 0.264 |
| 017A | 1.549 | 0.366 |
| 036A | 1.631 | 0.710 |
| 042A (negative control) | −0.086 | 0.001 |
| 042A + SEQ ID NO: 360 fungal | 0.317 | 0.087 |
| enzyme (0.09 mg/ml) | | |
| 042A + MegaTr. (0.25 mg/ml) | 3.226 | 0.083 |
| SEQ ID NO: 360 fungal enzyme | 1.565 | 0.734 |
| (0.09 mg/ml) | | |
| Megazyme TrCBH1 fungal | 3.719 | 0.831 |
| enzyme (0.05 mg/ml) | | |
| Buffer only | 0 | 0 |

Mega Tr. = commercially available CBH1, Megazyme TrCBH1

### Example 14

### β-Glucosidase Activity Assays

In one embodiment, isolated, synthetic or recombinant enzymes or other polypeptides of the invention have a β-glucosidase activity. This example describes an exemplary assay designed to measure activity of β-glucosidase enzymes on pNP-β-D-glucopyranoside substrate. This exemplary assay can be used to determine if a polypeptide has β-glucosidase, and is within the scope of this invention.

Enzyme activity is described in U/mL. If the protein concentration (mg/mL) is available, this value could be used to calculate enzyme specific activity (U/mg).

Unit Definition

One unit of activity is defined as the quantity of enzyme required to liberate one μmole of p-Nitrophenol per minute under the defined assay conditions (e.g. pH and temperature).

### Protocol (the Exemplary Polypeptide SEQ ID NO:560 is Used as an Example):

1. Use clear 96-well plate. Position all samples for fast, simultaneous addition of reaction components and to provide the shortest interval for a kinetic read. Run all samples in duplicate. Include standard curve (in duplicate) on each plate.
2. Standard curve preparation. Dilute 10 mM p-NP solution in 50 mM sodium phosphate buffer pH 7.0. Make at least 500 μL of each dilution: 0, 0.0625, 0.125, 0.25, 0.5, and 1.0 mM.
3. Sample preparation. For each enzyme sample to be tested, first assay the undiluted sample. If a dilution of the sample is required, make serial dilutions in 50 mM sodium phosphate buffer pH 7.0. Prepare at least 100 μL of each dilution.
4. Substrate preparation. To avoid substrate limitation in the enzymatic reaction, amount of pNP-β-D-glucopyranoside should be empirically determined for each enzyme which will be assayed. For example, for SEQ ID NO:560, substrate should be used at 20 mM final concentration. Final reaction volume will be 200 μL (see step 9). For each enzyme sample and each dilution to be tested prepare 190 μL solution consisted of 150 μL 50 mM sodium phosphate buffer pH 7.0 and 40 μl 100 mM pNP-β-D-glucopyranoside stock substrate (20 mM final concentration). For example, if 4 different dilutions of 2 enzyme sample will be tested in duplicate, prepare 3.8 mL of solution consisted of 3.0 mL 50 mM sodium phosphate buffer pH 7.0 and 0.8 mL 100 mM pNP-β-D-glucopyranoside solution. Preincubate solution for 5 min at 37° C. in a water bath and place into a pipetting basin immediately before dispensing it into a 96-well plate (see Step 6).
5. Load the standard curve. Preincubate standard curve dilution samples in a water bath at 37° C. for 5 min. Load, in duplicate, at 200 μL/well onto a clear 96-well plate. Put plate on SPECTRAMAX™ tray, lid off.
6. Load substrate. Quickly transfer the solution prepared in Step 4 into a pipetting basin and load substrate prepared in Step 4 in duplicate at 190 μL/well onto a clear 96-well plate. Put plate on SPECTRAMAX™ tray, lid off.
7. Equilibrate at 37° C. before kinetic read: Incubate 96-well plate containing the standard curve and substrate for 1 min at 37 C before adding enzyme samples.
8. Set up the SPECTRAMAX™. Set for kinetic read at 37 C. Choose the following SPECTRAMAX™ settings: (1) Absorbance 405 nm (A₄₀₅); (2) Timing: Take readings for total of 10 minutes with minimal allowed interval between the reads (this will depend on the number of strips which will be read on each plate); (3) Automixing & Blanking: “Automixing before the first read” should be “on”; “Automixing between reads” and “blanking before a pre-read” should be “off”; (4) “Autocalibrate once”: this function should be “on”; (5) Strips: select to read only strips where samples were loaded; (6) “Auto Read”: this function should be “off”. Make sure the standard curve samples are included in the kinetic read so that they are read under the same conditions.
9. Add enzyme and start kinetic read. Quickly add 10 μL of each enzyme dilution (in duplicate) to the wells containing 190 μl of solution dispensed in Step 6. Use a multichannel pipet for fast, simultaneous addition and mixing of samples. Immediately start kinetic read; save data. Make sure Vmax is calculated by the program in mU per minute.

### Calculations:

### Standard Curve

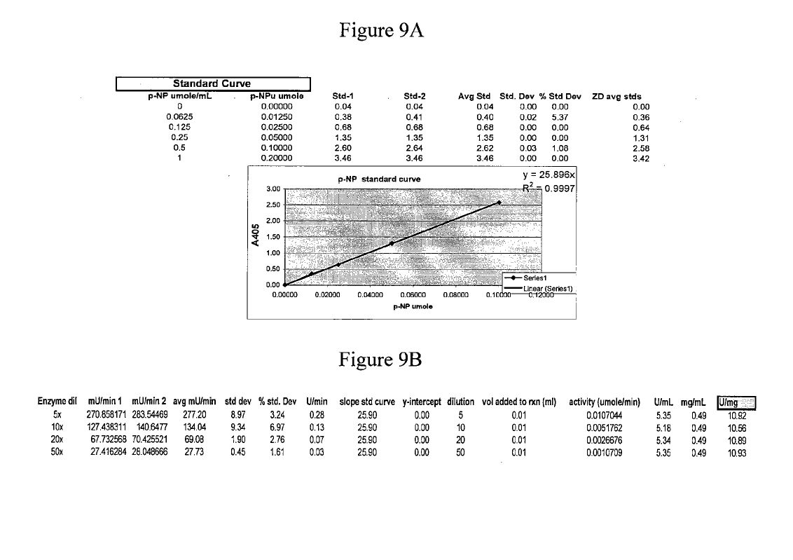

1. Use the first kinetic read to gather data for standard curve (the standard curve is just an endpoint measurement). First convert the mM values to μmole; for example, 200 μL, of 1.0 mM p-NP=0.2 μmole. For each point on the standard, calculate average and standard deviation for the duplicate samples and subtract the background from the average RFU values. Minimum of 4 data points are required to generate a reliable standard curve.
2. Generate a scatter plot using the background-subtracted average values, with μmol p-NP on the x-axis and A₄₀₅ on the y-axis.
3. Use linear regression to generate the line function relating A₄₀₅ to μmol p-NP present. Since background was subtracted, force y-intercept to 0. If the R-value is below 0.998, try omitting the data point representing the highest concentration on the standard curve. Nevertheless, minimum of 4 data points must be included in a standard curve. In MS EXCEL™, format the line function in scientific notation with 2 decimal points. Example of a standard curve is shown in FIG. 9A.

### Enzyme Activity

1. For each measurement of β-glucosidase activity, use the dilution that best fits the sensitivity range of the standard curve. Within this dilution, use only the data points that fall within the linear region of the kinetic to generate the Vmax (Vmax is automatically calculated in the SPECTRAMAX™ software (in mU/min)). Calculate the average and standard deviation for each pair of duplicates. Standard deviation should be no more than 5%. Then divide the average mU/min by 1000 to get U/min.

2. Use the line function to translate Vmax U/min value to units of β-gluc activity in μmol/min (U/min divided by slope of the standard curve), then divide that value by the volume of enzyme added to reaction in each well (0.01 mL), and finally multiply by the enzyme dilution factor to translate μmol/min value to U/mL value.

3. To obtain specific activity in U/mg of protein divide U/ml value by the protein concentration (mg/mL).

4. FIG. 9B shows how calculations can be set up in EXCEL™, given a standard curve slope of 25.90 (SEQ ID NO:560 and 20 mM pNP-β-D-glucopyranoside used in the assay).

### Reagents

p-NP Standard

p-nitrophenol (4-Nitrophenol) (Sigma N7660, 10 mM solution). Store at 4° C. protected from light. Make dilutions for standard curve in the buffer which will be used in the assay (e.g. 50 mM sodium phosphate pH 7.0).

pNP-β-D-Glucopyranoside Substrate (pNP-G)

pNP-β-D-glucopyranoside (FW 301.3 g/mol, Sigma N-7006). To make 10 ml of the 100 mM stock solution, weigh 0.3013 g of powder to a 15 mL conical centrifuge tube and dissolve the powder in 5.0 mL DMSO. Adjust the final volume to 10 mL with DIH₂O. Vortex the tube for several minutes to ensure solution in thoroughly mixed. Solution should be clear. If it is cloudy, heat the solution in a hot water bath and vortex gently until all material is solubilized. Aliquot and store at −20° C. protected from light.

### Protocol (the Exemplary Polypeptide SEQ ID NO:564 is Used as an Example):

This exemplary assay is for determination of β-glucosidase activity using pNP-β-D-glucopyranoside substrate at assay conditions of pH 5.0 and 37° C. Enzyme activity is described in U/mL. If the protein concentration (mg/mL) is available, this value could be used to calculate enzyme specific activity (U/mg).

1. Use clear 96-well plate and plan to position all samples for fast, simultaneous addition of reaction components and to provide the shortest interval for a kinetic read. Run all samples in duplicate. Include standard curve in duplicate on the plate as well.

2. Standard Curve Preparation. Dilute 10 mM p-NP solution in 50 mM sodium phosphate buffer pH 5.0. Make at least 500 μL of each of the following dilutions: 5, 2.5, 1.25, 0.625, 0.3125, 0.15625, and 0.078125 mM. Note: 1.0 mM=1.0 μmole/mL.

3. Sample Preparation. For each enzyme sample to be tested, first assay the undiluted sample. If a dilution of the sample is required, make serial dilutions in 50 mM sodium phosphate buffer pH 5.0. For example, purified SEQ ID NO:564 should be diluted 100 fold. Prepare at least 100 uL of each enzyme dilution; 50 μl of sample will be used per reaction.

4. Substrate preparation. Stock concentration of 100 mM is available for use. To avoid substrate limitation in the enzymatic reaction, amount of pNP-β-D-glucopyranoside should be empirically determined for each enzyme which will be assayed. For example, enzyme SEQ ID NO:564 should be assayed with 20 mM substrate. The final reaction volume for each sample to be assayed will be 1 mL.

For example, the following is required for a sample:
(i) 200 μl of 100 mM substrate.
(ii) 750 μl of 50 mM sodium phosphate buffer pH 5.0
(iii) 50 μl of diluted enzyme

5. Quencher Plate Preparation. Since the assay is performed at pH 5.0, a quenching step is required to appropriately read absorbance. Set up a clear 96-well plate with 200 μL of 400 mM Na₂CO₃ pH 10.0 in each well. Add 50 μL of each standard dilution in duplicate in the first two columns of each assay plate.

6. Preincubation. In a 1.5 mL Eppendorf tube, add 750 μl of buffer and 50 μl of enzyme and incubate at 37° C. for 5 minutes. Separately, incubate substrate at 37° C. also.

7. Starting the reaction. Use a timer and take aliquots of the sample at the following time points: 0, 2, 4, 8, 18, 28, 38, and 48 minutes. To initiate the reaction, add 200 μl of substrate in the reaction tube containing buffer and enzyme. Mix thoroughly and immediately take 50 μl aliquots and add to quencher plate in duplicate for the first timepoint, t=0 mins. Do the same at each time point. When a time point is taken, immediately replace the reaction tube at 37° C. Observe the color change in the quencher plate for all time points.

8. Set up the SpectraMax. Set for Endpoint read at 37° C. Choose the following SpectraMax settings: (1) Absorbance 405 nm (A₄₀₅); (2) Strips: select to read only strips where samples were loaded. Load plate onto the machine and hit Read. Save data once read is complete.

### Calculations

### Standard Curve

1. Use the endpoint read to gather data for calculating a standard curve. First convert p-NP dilutions (mM) to p-NP in μmole by multiplying with 0.05 (volume added into quencher plate); for example, 0.05 mL of 1.0 mM p-NP=0.05 μmole. For each standard dilution, calculate average and standard deviation for the duplicate samples and subtract the background from the average absorbance values. A minimum of 4 data points are required to generate a reliable standard curve.

2. Generate a scatter plot using the background-subtracted average values, with μmol p-NP on the x-axis and A₄₀₅ on the y-axis.

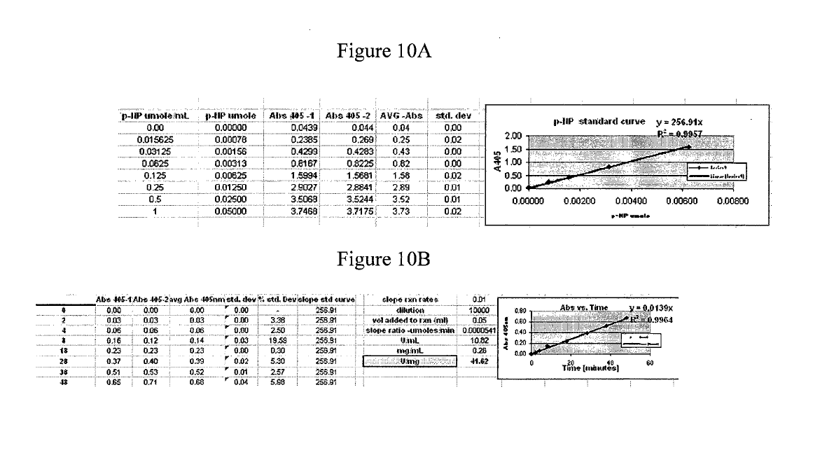

3. Use the linear regression trend line to generate the straight-line function relating A₄₀₅ to μmol p-NP present. Since background was subtracted, force y-intercept to 0. If the R-value is below 0.998, try omitting the data point representing the highest concentration on the standard curve. Nevertheless, minimum of 4 data points must be included in a standard curve. In MS Excel, format the line function in scientific notation with 2 decimal points. Example of a standard curve is shown in FIG. 10A.

### Calculating Enzyme Specific Activity

For the measurement of β-glucosidase activity, use the dilution that best fits the sensitivity range of the standard curve. Use the endpoint read to gather data for calculating specific activity for each enzyme.

1. For each enzyme dilution, at each timepoint, calculate average and standard deviation for the duplicate samples and subtract the background (absorbance at time point 0 min) from the average absorbance values. Standard deviation should be no more than 5%. An absorbance above 1.00 should not be used in calculations, as it does not lie in the reliable range of absorbance data. Again, a minimum of 4 data points are required to generate a reliable reaction rate curve.

2. Generate a scatter plot using the background-subtracted average values, with time in minutes on the x-axis and A₄₀₅ on the y-axis.

3. Use the linear regression trend line to generate the straight-line function relating A₄₀₅ over a 48 minute time course. Since background was subtracted, force y-intercept to 0. If the R-value is below 0.998, try omitting the data point that may be an outlier.

4. Then create a table, as shown below:
a. Slope [Δ Abs/min]−reaction rate
b. Final dilution of enzyme−initial enzyme dilution*(100)
c. Volume of enzyme added to reaction−0.05 mL
d. Slope ratio [μmole/min]−(slope of reaction rate/slope of standard curve)
e. Enzyme activity [U/mL]−(slope ratio/vol. enzyme in reaction)*dilution factor
f. Protein Concentration [mg/mL]−values from A₂₈₀ scan
g. Specific Activity [U/mg]−(enzyme activity/protein concentration)

5. FIG. 10B shows how calculations can be set up in EXCEL™, given a standard curve slope of 256.91 (SEQ ID NO:564—100× and 20 mM pNP-β-D-glucopyranoside used in the assay).
The standard curve and the endpoint absorbances should be measured on the SpectraMax together in the same read so that the sensitivity between the standard and sample is matched.
Determine target range for p-NP to get linear absorbance. Always make fresh dilutions of the p-NP standard.
Use the same buffer pH 5.0 for the standard curve and the sample measurements. p-NP is highly sensitive to changes in temperature and pH.

### Reagents

p-NP Standard

p-nitrophenol (4-Nitrophenol) (Sigma N7660, 10 mM solution). Store at 4° C. protected from light. Make dilutions for standard curve in the buffer which will be used in the assay (e.g. 50 mM sodium phosphate pH 5.0).

pNP-β-D-Glucopyranoside Substrate (pNP-G)

pNP-β-D-glucopyranoside (FW 301.3 g/mol, Sigma N-7006). To make 10 ml of the 100 mM stock solution, weigh 0.3013 g of powder to a 15 mL conical centrifuge tube and dissolve the powder in 5.0 mL DMSO. Adjust the final volume to 10 mL with distilled water (DIH₂O). Vortex the tube for several minutes to ensure solution in thoroughly mixed. Solution should be clear. If it is cloudy, heat the solution in a hot water bath and vortex gently until all material is solubilized. Aliquot and store at −20° C. protected from light.

### β-Glucosidase Evaluation

Sixty seven beta-glucosidases were partially characterized; 36 of these enzymes of this invention were further evaluated based on the following criteria: (1) activity on cellobiose and on fluorescent substrate 4-methylumbelliferyl beta-D-glucopyranoside (4-MU-GP) at pH5 and 7 and T=37-90 C; (2) expression in heterologous hosts; (3) residual activity at T=60-80 C, pH5; and (4) level of product inhibition. Eight (8) beta-glucosidases of this invention were identified based on the multiple selection criteria: including the exemplary polypeptides of this invention SEQ ID NO:556, SEQ ID NO:566, SEQ ID NO:530, SEQ ID NO:548, SEQ ID NO:564, SEQ ID NO:560, SEQ ID NO:586, and SEQ ID NO:558.
His-tag versions of these beta-glucosidases of this invention were generated and produced in E. coli by batch fermentation. Protein purification was completed for five (5) exemplary enzymes of this invention. Absorbance based activity assay using pNP-beta-D-glucopyranoside substrate (pNP-G) was developed and used to determine specific activity of exemplary enzymes of this invention. Megazyme I A. niger beta-glucosidase was used as benchmark in all experiments. Several enzymes had specific activities significantly higher than the commercial benchmark under chosen assay conditions.
To complete selection of beta-glucosidases which will be most suitable for use in multi-enzyme cocktails, product inhibition and residual activity at T=60-80 C can be determined for beta-glucosidases of this invention with high specific activities.

Characterization of Beta-Glucosidases

Introduction: The main objectives of this work were the following: (1) Identify β-glucosidase enzymes of this invention for use in a four enzyme cocktail which will be designed to meet MS1 criteria; (2) Identify several enzymes which could be used in a combinatorial assay.

Sixty seven beta-glucosidases were partially characterized. 36 of these enzymes were further evaluated based on the following criteria: (1) activity on cellobiose and on fluorescent substrate 4-methylumbelliferyl beta-D-glucopyranoside (4-MU-GP) at pH5 and 7 and T=37-90 C; (2) expression in heterologous hosts; (3) residual activity at T=60-80 C, pH5; and (4) level of product inhibition. The following 81 beta-glucosidases of this invention were identified based on multiple selection criteria: the exemplary polypeptides of the invention SEQ ID NO:556, SEQ ID NO:566, SEQ ID NO:530, SEQ ID NO:548, SEQ ID NO:564, SEQ ID NO:560, SEQ ID NO:586, and SEQ ID NO:558.

To enhance protein purification, His-tag versions of all exemplary beta-glucosidases of this invention (SEQ ID NO:556, SEQ ID NO:566, SEQ ID NO:530, SEQ ID NO:548, SEQ ID NO:564, SEQ ID NO:560, SEQ ID NO:586, and SEQ ID NO:558) were generated. Expression was evaluated in shake-flasks and optimal induction conditions were transferred to fermentation protocols. The exemplary enzymes were produced by batch fermentation (10 L scale), material was recovered and proteins were purified by the FPLC method. Protein concentration was determined by several methods, including Bradford assay, gel-densitometry and absorbance at A₂₈₀. Absorbance based activity assay using pNP-beta-D-glucopyranoside substrate (pNP-G) was developed. This assay was used to determine specific activity of beta-glucosidases of this invention. Commercial A. niger beta-glucosidase was used as benchmark in all experiments. To complete selection of beta-glucosidases which best fit the objectives outlined above, product inhibition and residual activity at T=60-80 C will be determined using purified protein preparations of enzymes of this invention with high specific activities.

Methods/Results:

(1) Protein Expression, Production, and Purification.

His-tag versions of all enzymes of this invention were generated by site-directed mutagenesis which removed a stop codon between the ORF and C′-terminal 6×-His tag in pSE420 vector in original subclones.

All enzymes except SEQ ID NO:558 were expressed in E. coli host (strains GAL631 and Rosetta Gami) and produced by 10 L batch fermentation. Several of these proteins were expressed in both soluble and insoluble fractions and were recovered from both supernatants (S) and cell pellets (P) after extensive troubleshooting. Designations “S” and “P” will be used next to relevant in the following sections where protein purification and activity assays will be presented. Expression of all enzymes of this invention was also evaluated in Pichia pastoris ×33 host (vectors pPICZα and pGAPα). All subclones produced soluble proteins, but expression and activities were too low to permit use of Pichia-produced material in enzyme characterization. SEQ ID NO:558 was originally generated in Pichia pastoris ×33 and showed good expression (35% purity), but also high level of product inhibition. His-tag version of SEQ ID NO:558 showed little expression in Pichia and was not further characterized.

FPLC protein purification was completed for 5 of 7 enzymes of this invention which were produced in E. coli (SEQ ID NO:548, SEQ ID NO:564, SEQ ID NO:560, SEQ ID NO:530 and SEQ ID NO:566). His-tagged enzyme constructs were used and 1 gram of lyophilized powder of each was resuspended in 10.0 mL of Buffer A. (20 mM Tris-HCl, 500 mM NaCl, pH8.0) and dialyzed in the same buffer overnight. Following dialysis, the 10 mL of each sample was loaded onto a HISTRAP™ 5 mL column and eluted using the HISTRAP™ 5 mL protocol. The HISTRAP™ method calls for washing the unbound protein with 5 column volumes of Buffer A and eluting the bound proteins over 20 column volumes using a linear gradient of Buffer B (20 mM Tris, 500 mM NaCl, 1 M imidazole, pH8.0). A flow rate of 2 mL/min was maintained during the procedure and 1 mL elution fractions were collected in 96-deep well plates. Based on the FPLC data, optimal fractions, those lying under the chromatogram peaks, were selected to test for enzyme activity and expression/purity. Activity was tested using the 4-MU-GP fluorescent assay. Selected fractions were also run on PAGE gels to test for purity

Summary of fermentation recovery and protein purification efforts is shown in Table 1. All of these exemplary enzymes of this invention were generated in enough quantities (“total lyophilized powder” and “expected mg of purified protein in 1 g of powder and in total lot”) to allow in-depth biochemical characterization of relevant enzymes.

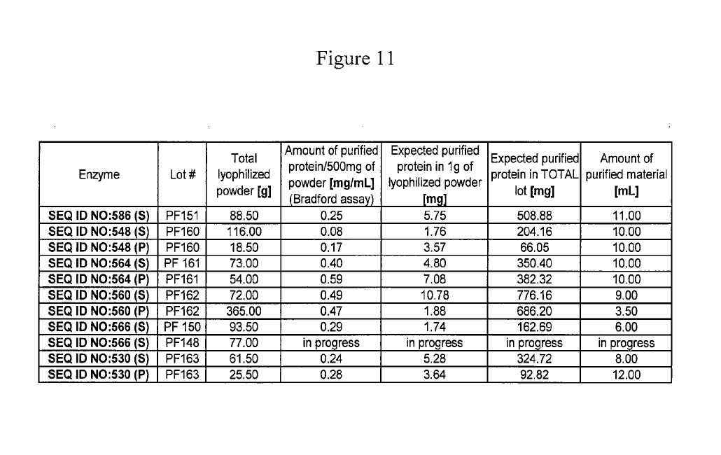

Table 1, illustrated as FIG. 11, shows data from the production and purification summary for beta-glucosidase enzymes of this invention.

(2) Determination of Protein Concentration.

Three methods were used to determine concentration of purified proteins, Bradford assay, gel densitometry and absorbance at A₂₈₀ nm. All FPLC-purified proteins were dialyzed in 50 mM NaPi pH 7.0 before activity characterization. Results are summarized in the table of FIG. 11.

The Bradford colorimetric assay measures protein absorbance at 595 nm. Purified protein samples were added to assay dye reagent, color change was observed, and absorbance at 595 nm was read. BSA protein standard was used at 0.05, 0.1, 0.2, 0.3, 0.4, and 0.5 mg/mL to determine the unknown concentrations. Protein concentrations of enzymes are extrapolated from a ‘line-of-best fit’ linear curve generated with BSA standards.

To evaluate protein purity and to determine concentration by gel densitometry, 5 μL of enzyme was loaded per lane on 4-12% Tris-Glycine PAGE gel (Invitrogen) and electrophoresis was performed in 2× MES buffer at 200 V for 50 min. Amount of protein loaded for each sample is noted below. BSA protein standard (Pierce) was loaded on all gels in 0.25, 0.50, 1.00, 1.50, and 2.00 μg total quantities per lane. Gels were scanned using ALPHAIMAGER® software and protein concentrations were determined based on the BSA standard curves.

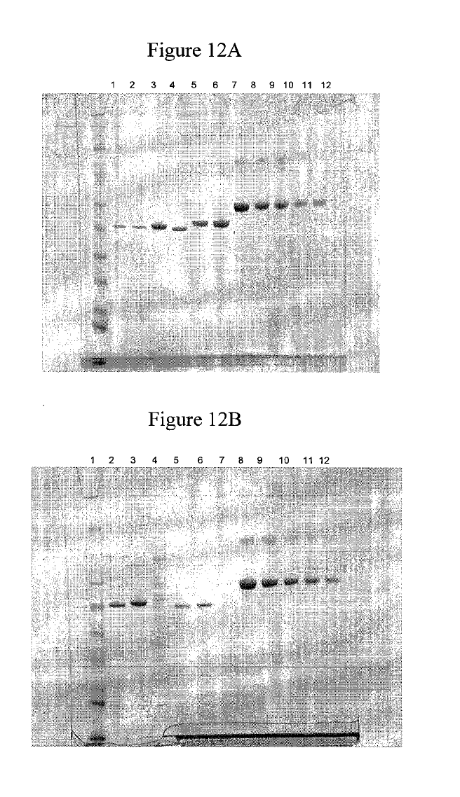

FIG. 12A: PAGE electrophoresis of SEQ ID NO:548, SEQ ID NO:564, and SEQ ID NO:560 purified from supernatant and pellet cell fractions by the FPLC method:

Lane 1—SeeBlue® marker Plus Two ladder Invitrogen)

Lane 2—SEQ ID NO:548 (P)—0.07 μg

Lane 3—SEQ ID NO:548 (S)—0.12 μg

Lane 4—SEQ ID NO:564 (P)—0.23 μg

Lane 5—SEQ ID NO:564 (S)—0.41 μg

Lane 6—SEQ ID NO:560 (P)—0.54 μg

Lane 7—SEQ ID NO:560 (S)—0.37 μg

Lane 8—BSA standard—2.0 μg

Lane 9—BSA standard—1.5 μg

Lane 10—BSA standard—1.0 μg

Lane 11—BSA standard—0.50 μg

Lane 12—BSA standard—0.25 μg

FIG. 12B: PAGE electrophoresis of SEQ ID NO:530 and SEQ ID NO:566 purified from supernatant and pellet cell fractions by the FPLC method:

Lane 1—SeeBlue marker Plus Two ladder (Invitrogen)

Lane 2—SEQ ID NO:530 (P)—0.18 μg

Lane 3—SEQ ID NO:530 (S)—0.12 μg

Lane 4—N/A

Lane 5—SEQ ID NO:566 (S)—0.07 μg

Lane 6—N/A

Lane 7—BLANK

Lane 8—BSA standard—2.00 μg

Lane 9—BSA standard—1.50 μg

Lane 10—BSA standard—1.00 μg

Lane 11—BSA standard—0.50 μg

Lane 12—BSA standard—0.25 μg

When absorbance at A₂₈₀ nm was used to determine protein concentration, 1 mL of material shown in Table 1, illustrated as FIG. 11, was placed in a quartz cuvette and scanned at A₂₀₀-A₃₂₀ nm at 5 nm range. The A₂₈₀ values and molar extinction coefficients (εₘ), which were determined based on individual amino acid sequences using Vector NTi software, were used to calculate protein concentration (C=mg/mL; C=(A₂₈₀×MW/(εₘ)); MW=protein molecular weight). Ratio A₂₆₀/A₂₈₀ was calculated and used to estimate protein purity (ratio approximately 0.5 to 0.7 is expected for highly pure proteins). *Samples shown in Table 1 (FIG. 11) were concentrated.

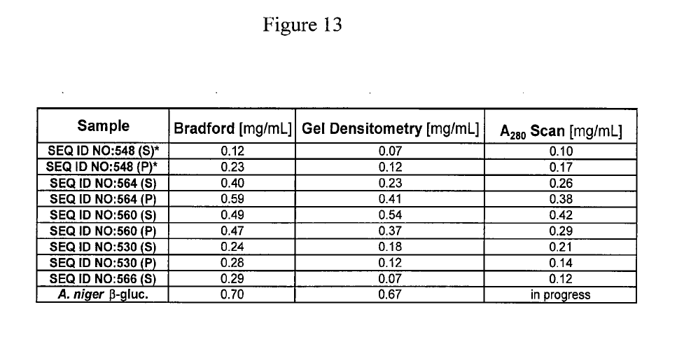

Table 2, illustrated as FIG. 13, shows protein concentrations of purified Beta-glucosidases determined by the three different methods:

Values (mg/mL) obtained with the three independent methods described above were in agreement for most of the samples, especially when numbers determined by gel densitometry and absorbance at A₂₈₀ were compared. Since these three methods use different parameters to estimate protein concentration, and given that Bradford assay tends to over-estimate it, mg/mL values determined by absorbance at A₂₈₀ will be used for calculation of protein specific activity.

(3) Determination of Beta-Glucosidase Specific Activity.

Absorbance based activity assay using pNP-beta-D-glucopyranoside substrate (pNP-G) was developed and used to determine specific activity of enzymes of this invention. One unit of activity is defined as the quantity of enzyme required to liberate one μmole of p-Nitrophenol per minute under defined assay conditions. Enzyme activity is described in U/mL. When protein concentration (mg/mL) is known, enzyme specific activity (U/mg) could be determined using these values.

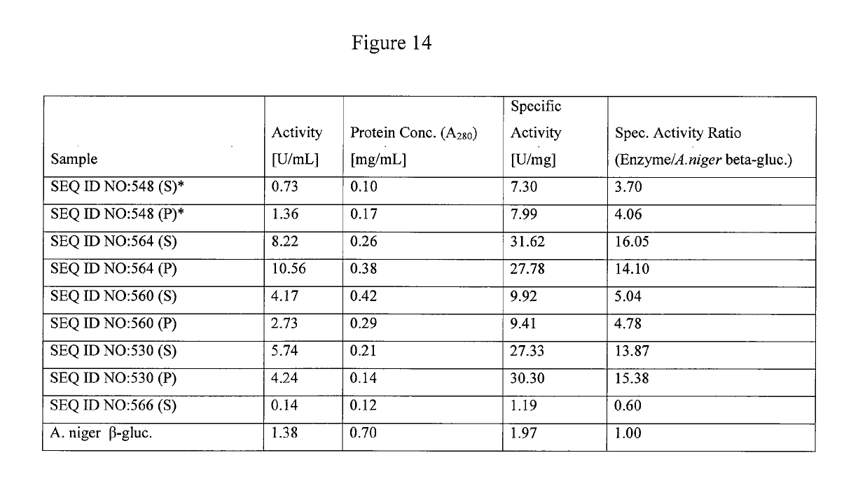

For thorough activity testing, purified protein preparations were diluted in serial fashion and incubated at pH 7 for 10 min with 20 mM pNP-G at T=37° C. Commercial A. niger beta-glucosidase was used as benchmark. Standard curves were generated using 10 mM p-Nitrophenol (p-NP) diluted in 50 mM Na-phosphate buffer pH 7 to 0.0625, 0.125, 0.25, 0.5 and 1 μmole/mL. Enzyme loading was adjusted to obtain absorbance which will remain within linear range of the p-NP standard curve in the detection assay. Enzyme activity was determined and expressed in U/mL. Protein concentration determined by absorbance at A₂₈₀ was used to calculate specific activity. Results summary is shown in Table 3, illustrated as FIG. 14.

Under chosen assay conditions, four of the five enzymes characterized to date showed specific activities higher than a commercial benchmark (Table 3/FIG. 14). SEQ ID NO:548 and SEQ ID NO:560 exhibited specific activities about 4-5-fold higher compared to the benchmark. SEQ ID NO:564 and SEQ ID NO:530 outperformed the benchmark approximately 15-fold. No significant difference was observed when enzymes were purified from soluble or insoluble cell fractions. The pNP-G assay was run at pH 7 for practical reasons, given that beta-glucosidases of the invention show activity over broad pH spectrum. However, since A. niger beta-glucosidases shows optimal activity at lower pH, specific activity comparison will be repeated at pH 5. *Sample shown in Table 1 was concentrated.

Table 3, or FIG. 14, shows the specific activities of purified beta-glucosidases of this invention.

### Summary

Five beta-glucosidase enzymes of this invention were produced by batch fermentation and proteins were purified by the FPLC method. Protein concentration was determined by several methods, including Bradford colorimetric assay, gel-densitometry and absorbance at A₂₈₀. Specific activity was determined for the five beta-glucosidases of this invention and compared to a commercial prep of A. niger beta-glucosidase, which was purchased from Megazyme and used as a benchmark.

Four of five enzymes characterized up to date outperformed the commercial benchmark under chosen assay conditions (Table 3). Two of these enzymes (SEQ ID NO:564 and SEQ ID NO:530) exhibited specific activities about 15-fold higher than the benchmark. SEQ ID NO:548 and SEQ ID NO:560 exhibited specific activities about 4-5-fold higher when compared to the benchmark. Non-His tag versions of SEQ ID NO:564, SEQ ID NO:548, and SEQ ID NO:530 (SEQ ID NO:564, SEQ ID NO:548, and SEQ ID NO:530) showed product inhibition in the presence of 500, 400 and 200 mM glucose, respectively, when semi-purified proteins were evaluated on cellobiose substrate.

Accurate determination of protein concentration proved to be quite challenging. However, values (mg/mL) obtained with the three independent methods used in this study were in agreement for most of the samples, especially when numbers determined by gel densitometry and absorbance at A₂₈₀ were compared (Table 2). Since the three methods use different parameters to estimate protein concentration, and given that Bradford assay tends to over-estimate it, mg/mL values determined by absorbance at A₂₈₀ were used for calculation of protein specific activity.

Further Characterization of Beta-Glucosidases of this Invention

The main objectives of the studies described in this example were: (1) Identify exemplary β-glucosidase enzymes of this invention for use in multi-enzyme cocktails which will be designed to effectively degrade cellulose component of pretreated sugar cane bagasse; (2) Identify several enzymes of this invention to use in high-throughput combinatorial assays.

Sixty seven (67) beta-glucosidases were partially characterized, and 36 of these enzymes were further evaluated based on the following criteria: (1) activity on cellobiose and on fluorescent substrate 4-methylumbelliferyl beta-D-glucopyranoside (4-MU-GP) at pH5 and 7 and T=37-90 C; (2) expression in heterologous hosts; (3) residual activity at T=60-80 C, pH5; and (4) level of product inhibition by glucose. The following 8 enzymes of this invention were identified based on multiple selection criteria: the exemplary enzymes of this invention SEQ ID NO:556, SEQ ID NO:566, SEQ ID NO:530, SEQ ID NO:548, SEQ ID NO:564, SEQ ID NO:560, SEQ ID NO:586, and SEQ ID NO:558.

To enhance protein purification, His-tag versions of the 8 exemplary enzymes of this invention were generated and produced in E. coli by batch fermentation (10 L scale). Protein purification was successfully completed for 5 of 8 exemplary enzymes of this invention (SEQ ID NO:548, SEQ ID NO:564, SEQ ID NO:560, SEQ ID NO:566, and SEQ ID NO:530) and specific activity at pH7 was determined using absorbance based activity assay and pNP-beta-D-glucopyranoside substrate (pNP-G). Commercial A. niger beta-glucosidase was used as benchmark. Four enzymes were identified which showed specific activities at pH 7 significantly higher than the commercial benchmark (the exemplary enzymes of this invention SEQ ID NO:548, SEQ ID NO:564, SEQ ID NO:560 and SEQ ID NO:530).

Additional biochemistry characterization was performed with 5 exemplary enzymes of this invention, beta-glucosidases, to select final candidates which are suitable for use in multi-enzyme cocktails and in high-throughput combinatorial assays. Specific activity at pH 5, product inhibition in the presence of glucose (0 to 2M), residual activity at 60° C., and pH profile (specific activity at pH 4 to 8) were determined as described herein.

Methods/Results:

(1) Determination of Beta-Glucosidase Specific Activity at pH 5.

Absorbance based activity assay using pNP-beta-D-glucopyranoside substrate (pNP-G) was developed and used to determine specific activity of exemplary enzymes of this invention. One unit of activity is defined as the quantity of enzyme required to liberate one mole of p-Nitrophenol per minute under defined assay conditions. Enzyme activity is described in U/mL. When protein concentration (mg/mL) is known, enzyme specific activity (U/mg) could be determined using these values.

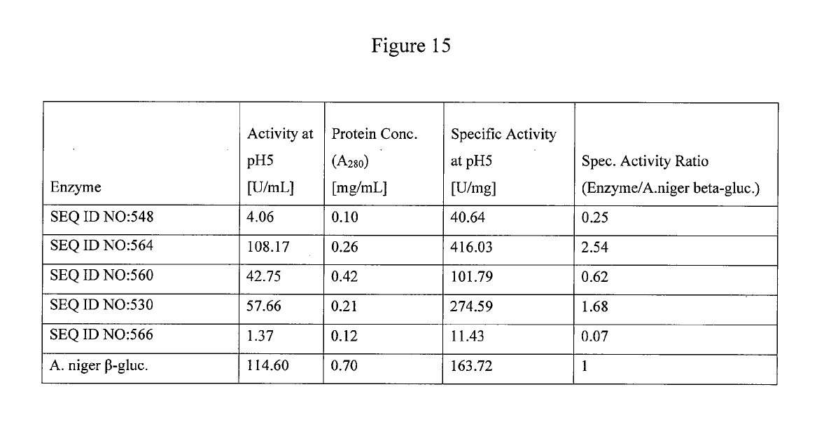

For thorough activity testing, purified protein preparations were diluted in serial fashion in 50 mM Na-phosphate buffer pH 5 and incubated with 20 mM pNP-G (1 mL total reaction volume) at T=37° C. for 48 min. 50 μl aliquots were taken at time points 0, 2, 4, 8, 18, 28, 38 and 48 min and added to a quencher 96-well plate containing 200 μl of 400 mM Na₂CO₃ pH 10 in each well. Commercial A. niger beta-glucosidase was used as benchmark. Standard curves were generated using 10 mM p-Nitrophenol (p-NP) diluted in 50 mM Na-phosphate buffer pH 5 to 0.015625, 0.03125, 0.0625, 0.125, 0.25, 0.5 and 1.0 μmole/mL. Absorbance was measured at 405 nm (end-point read). Enzyme loading was adjusted to obtain absorbance which will remain within linear range of the p-NP standard curve in the detection assay. Enzyme activity was determined and expressed in U/mL. Protein concentration determined by absorbance at A₂₈₀ was used to calculate specific activity. Results are summarized in Table 1, illustrated as FIG. 15.

Two of five selected enzymes of the invention (SEQ ID NO:564 and SEQ ID NO:530) exhibited higher specific activities at pH 5 compared to A. niger beta-glucosidase. The other 3 exemplary enzymes of this invention had lower specific activities at pH 5 compared to benchmark (Table 1/FIG. 15).

Table 1, illustrated as FIG. 15, shows the specific activity of exemplary beta-glucosidases of this invention.

(2) Product Inhibition in the Presence of Glucose.

The objective of this work was to evaluate beta-glucosidases of this invention for levels of product inhibition and enzyme activity in the presence of various concentrations of glucose. Glucose was dosed at 0 to 300 mM (“low dose”) and 0 to 2 M (“high dose”) and specific activity on p-NPG substrate was determined as described in the previous section (48 min reactions at 37° C. and pH 5). Glucose concentrations were chosen based on process assumptions (hydrolysis at 20% solids) and previous results obtained in product inhibition experiments performed with semi-purified enzymes. For each enzyme, specific activity in the absence of glucose was designated as 100%.

All 5 exemplary enzymes of this invention retained activity at pH 5 in the presence of all glucose concentrations evaluated in this experiment and significantly outperformed A. niger benchmark. In a commercial process which would assume hydrolysis of bagasse (approximately 50% cellulose content) at 20% solids and 50% enzymatic conversion, approx. 280 mM glucose (50 g/L) would be generated from 1 kg of starting material (equivalent of a “low-dose” evaluated in this experiment). All 5 exemplary enzymes of this invention tested in this study retained ≧50% activity in the presence of 300 mM glucose, compared to <25% retained by the commercial benchmark. Similar trend was observed in the presence of higher glucose concentrations. For example, accumulation of ≧1M glucose would be expected in a process that would run at ≧50% solids which would be very difficult to achieve in practice. However, even under such harsh conditions, all 5 exemplary enzymes of this invention retained ≧20% activity while commercial benchmark retained <5% activity.

Increase in specific activity of SEQ ID NO:560 and SEQ ID NO:566 in the presence of glucose was repeatedly observed in multiple experiments. This phenomenon is not understood, and it will not be further investigated at this time.

(3) Residual Beta-Glucosidase Activity at 60° C.

Four (4) exemplary beta-glucosidases of this invention (SEQ ID NO:548, SEQ ID NO:564, SEQ ID NO:560 and SEQ ID NO:530) and commercial A. niger beta-glucosidase were incubated for 0-4 hours at 60° C. at pH 5 (1 mL reaction volume, 300 rpm mixing). Aliquots were removed at every hour and assayed for residual enzyme activity on p-NPG substrate as described in Section (1) of this Report. For each enzyme, specific activity at Time=0 h was designated as 100%.

3 of 4 tested enzymes (SEQ ID NO:548, SEQ ID NO:564 and SEQ ID NO:530) showed significant loss of activity after exposure to 60° C. for extended periods of time (≧2 h). These enzymes lost ≧50% activity after 4 h at 60° C., while commercial benchmark retained about 70% activity. This finding is contradictory to previous results generated with crude protein preparations (lyophilized bacterial lysates) assayed on cellobiose and on fluorescent substrate. When assayed as crude enzymes (protein of interest present ≦10% total protein), all 4 enzymes retained ≧90% activity after 4 h at 60 C. It is possible that residual host proteins could provide some stabilization effect at elevated temperatures. However, this finding further emphasizes the need to generate purified protein preparations in order to perform meaningful enzyme characterization.

(4) Determination of pH Profile of Exemplary Beta-Glucosidases.

Specific activity of five (5) exemplary enzymes of this invention, the beta-glucosidases SEQ ID NO:548, SEQ ID NO:564, SEQ ID NO:560, SEQ ID NO:566, and SEQ ID NO:530, and commercial A. niger beta-glucosidase was determined on p-NPG substrate at pH 4, 5, 6, 7, and 8. Assays performed at pH 4 and 5 were run as described above, except that 50 mM Na-phosphate buffer was adjusted to pH 4 and 5. Assays performed at pH 6, 7, and 8 were run as described above. Briefly, purified protein preparations were diluted in serial fashion and incubated at pH 6, 7, and 8 for 10 min with 20 mM pNP-G at T=37° C. Commercial A. niger beta-glucosidase was used as benchmark. Standard curves were generated using 10 mM p-Nitrophenol (p-NP) diluted in 50 mM Na-phosphate buffer pH 6, 7, and 8 to 0.0625, 0.125, 0.25, 0.5 and 1 μmole/mL. Enzyme loading was adjusted to obtain absorbance which will remain within linear range of the p-NP standard curve in the detection assay. Enzyme activity was determined and expressed in U/mL. Protein concentration determined by absorbance at A₂₈₀ was used to calculate specific activity (U/mg). Specific activities determined over a pH 4-8 range are summarized in Table 2, below.

Based on the specific activities retained over a pH 4-8 range, all of the tested exemplary beta-glucosidases of this invention outperformed the benchmark. SEQ ID NO:564 and SEQ ID NO:530 show strong pH 5 optimum (Table 2, below) and a narrow pH profile (14-18% specific activity retained at pH6 and 11% specific activity retained at pH7). Benchmark enzyme shows strong pH 4-5 optimum and retains only 5% specific activity at pH 6 and 1% specific activity at pH7. SEQ ID NO:548, SEQ ID NO:560 and SEQ ID NO:566 show pH 4-5 profile more similar to the benchmark than to SEQ ID NO:564 and SEQ ID NO:530.

TABLE 2
Specific activity of beta-glucosidases and benchmark at pH 4-8:

| | pH | | | | |
|---|---|---|---|---|---|
| Enzyme | 4 | 5 | 6 | 7 | 8 |
| SEQ ID NO: 548 | 45.60 | 40.64 | 8.88 | 6.70 | 4.87 |
| SEQ ID NO: 564 | 162.14 | 416.03 | 57.97 | 44.52 | 35.41 |
| SEQ ID NO: 560 | 88.74 | 101.79 | 13.83 | 10.98 | 7.93 |
| SEQ ID NO: 530 | 159.47 | 274.59 | 50.29 | 30.11 | 12.69 |
| SEQ ID NO: 566 | 8.04 | 11.43 | 1.93 | 0.99 | 0.45 |
| A. niger β-gluc. | 177.69 | 163.72 | 9.45 | 1.78 | 0.66 |

Summary:

Selection of beta-glucosidases of this invention which in some embodiments and applications may the most suitable for use in multi-enzyme cocktails was completed. Five exemplary enzymes of this invention were selected were further characterized to determine their specific activity at pH 5, product inhibition in the presence of glucose (0-2M), residual activity at 60 C, and pH profile (specific activity at pH 4-8).

Selected enzymes of this invention strongly outperformed commercial benchmark (Megazyme A. niger beta-glucosidase) by all parameters considered, with the exception of residual activity after extended exposure to high temperatures (4 h at 60° C.). These enzymes show high specific activities at pH 5 and little product inhibition by glucose. Selected exemplary enzymes of this invention retain activity over a broad pH range and could be used in a variety of applications. Characterization summary of several beta-glucosidases is shown in Table 3, below:

TABLE 3
Characterization summary

| | SA at | SA at | % Activity | pH | % Res. |
|---|---|---|---|---|---|
| | pH5 | pH7 | (0.3 M | optima/ | activity (4 h |
| Enzyme | (U/mg) | (U/mg) | gluc.) | range | at 60° C.) |
| SEQ ID NO: 564 | 416 | 45 | 76 | 5/4-8 | 17 |
| SEQ ID NO: 530 | 275 | 30 | 50 | 5/4-8 | 25 |
| SEQ ID NO: 560 | 102 | 11 | >100 | 4-5/4-8 | 90 |
| A. niger | 164 | 2 | 18 | 4-5/4-6 | 70 |

SA, specific activity; Numbers shown in Table 1 and 2 rounded to 0 decimals.

Summary of Selected Beta-Glucosidase Studies

| Enzyme | | | | |
|---|---|---|---|---|
| (Nucleotide SEQ ID | | | His-tag | |
| NO:, amino acid | Selected for | Primary | version | Secondary |
| SEQ ID NO:) | characterization | hits | created | hits |
| 537, 538 | X | | | |
| 555, 556 | X | X | X | |
| 535, 536 | X | | | |
| 539, 540 | X | | | |
| 545, 546 | X | | | |
| 565, 566 | X | X | X | X |
| 549, 550 | X | | | X |
| 529, 530 | X | X | X | |
| 541, 542 | X | | | |
| 543, 544 | X | | | |
| 553, 554 | X | | | |
| 547, 548 | X | X | X | X |
| 563, 564 | X | X | X | X |
| 525, 526 | X | | | |
| 531, 532 | X | | | |
| 551, 552 | X | | | |
| 561, 562 | X | | | |
| 589, 590 | X | | | |
| 559, 560 | X | X | X | X |
| 591, 592 | X | | | |
| 527, 528 | X | | | |
| 533, 534 | X | | | |
| 567, 568 | X | | | |
| 593, 594 | X | | | |
| 595, 596 | X | | | |
| 571, 572 | X | | | |
| 583, 584 | X | X | | |
| 585, 586 | X | X | X | |
| 587, 588 | X | | | |
| 569, 570 | X | | | |
| 581, 582 | X | | | |
| 577, 578 | X | | | |
| 573, 574 | X | | | |
| 575, 576 | X | | | |
| 557, 558 | X | | X | |
| 579, 580 | X | | | |

### Example 15

### Characterization of Beta-Glucosidases of this Invention

The main objective of this work was to characterize exemplary β-glucosidases of this invention to identify the most suitable enzyme(s) which could be included in a 4-enzyme cocktail. Enzyme activity and residual stability at higher temperatures, good specific activity, and low or no product inhibition, were chosen as main selection criteria for ranking enzyme candidates. Sixty seven enzymes β-glucosidase collection were evaluated at different pH and temperatures using surrogate fluorescent substrate (pNP-B-Glucopyranoside). Fourteen enzymes of the invention were identified as best performers and selected for more detailed evaluation on cellobiose substrate. Ten of these enzymes have been evaluated to date. Semi-pure protein preparations from subclone fermentations were used in the analysis which involved (1) determination of enzyme activity under different pH and temperature conditions, (2) semi-quantitative analysis of specific-activity, and (3) determination of residual activity at temperatures up to 80° C. Analysis was carried out with several enzyme doses and substrate concentrations, and at pH and temperature conditions described in the following sections. Time points were taken over 0.5 h-4 h reaction times and the amount of released glucose was measured with the AMPLEX RED® glucose oxidase assay. PAGE (polyacrylamide gel electrophoresis) was carried out to estimate amount of proteins used in reactions.

Methods/Results:

(1) Activity of Beta-Glucosidases at pH 5 and pH 7 and at Temperature Range Between 37° C. and 90° C.

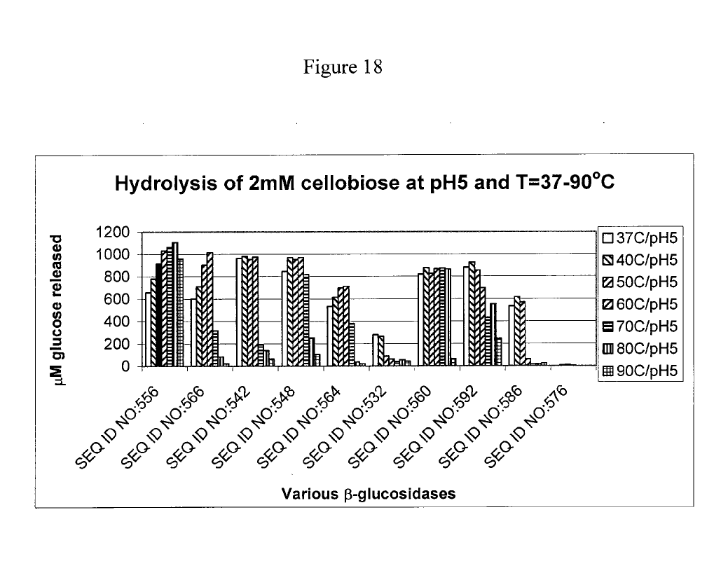

Semi-pure lyophilized protein preparations from subclone fermentations were reconstituted in 50% glycerol to obtain 25 mg/ml total protein concentration. Protein preps were subsequently diluted 40-fold (empirically determined to avoid enzyme inhibition by glycerol) and used in the reactions which were run for 1 hour at pH 5 and pH 7 and at 37, 40, 50, 60, 70, 80, and 90° C. 2 mM cellobiose substrate was used. Reaction volume was 50 μl (substrate and enzyme present at 1:1 ratio). The following 10 enzymes were evaluated in this experiment: SEQ ID NO:556, SEQ ID NO:566, SEQ ID NO:542, SEQ ID NO:548, SEQ ID NO:564, SEQ ID NO:532, SEQ ID NO:560, SEQ ID NO:592, SEQ ID NO:586, and SEQ ID NO:576. Results are shown in FIG. 18.

FIG. 18 illustrates data showing the hydrolysis of 2 mM cellobiose at different temperatures at pH 5 using exemplary enzymes of this invention.

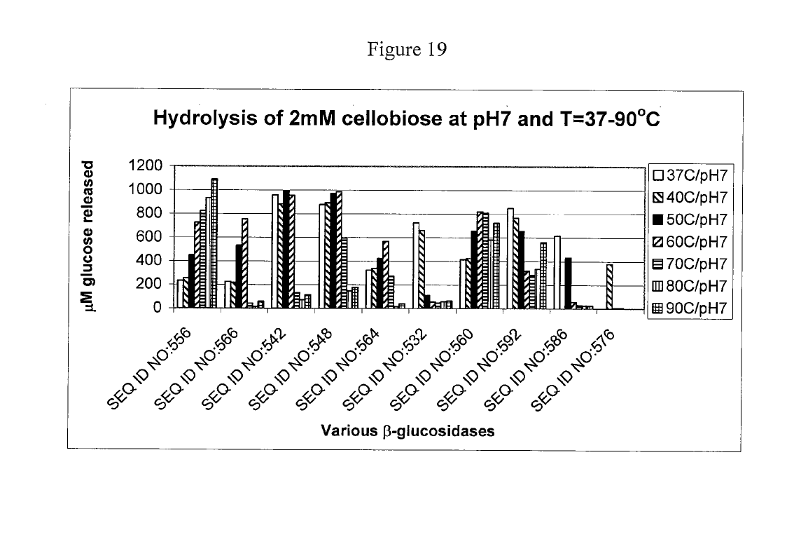

FIG. 19 illustrates data showing the hydrolysis of 2 mM cellobiose at different temperatures at pH 7 using exemplary enzymes of this invention.

The following was concluded based on the results obtained in this experiment:

(1) Most of the tested exemplary enzymes of this invention performed better at pH 5 than at pH 7;

(2) Several exemplary enzymes of this invention showed strong activity at pH 5 over the range of temperatures (SEQ ID NO:556, SEQ ID NO:566, SEQ ID NO:542, SEQ ID NO:548, and SEQ ID NO:560) and were selected for more detailed analysis;

(3) SEQ ID NO:556 remained very active at all temperatures and SEQ ID NO:560 was active up to 90° C.;

(4) Temperature 60° C. and pH5 were selected as reaction conditions for future experiments to mimic reaction conditions these enzymes will encounter in a 4-enzyme cocktail.

(2) Semi-Quantitative Determination of Specific Activity of Selected Beta-Glucosidases of this Invention.

Accurate determination of specific activity was not practical since semi-pure protein preparations were used in experiments described here (purified proteins were not available for this initial screen). To determine specific activity in a semi-quantitative manner, multiple dilutions were prepared for each of the selected enzymes and cellobiose digestion was performed for 16 min at pH 5 and 60° C. using 10 mM substrate (amount determined empirically to avoid substrate limitation). Reaction volume was 50 μl (substrate and enzyme present at 1:1 ratio). The objective was to achieve comparable rate kinetics for all enzymes at selected reaction conditions. PAGE was run in parallel to estimate amount of beta-glucosidases in each protein preparation. Enzyme with the least amount of protein in the prep (most diluted) and having the rate comparable to others was considered to have the best specific activity under test conditions.

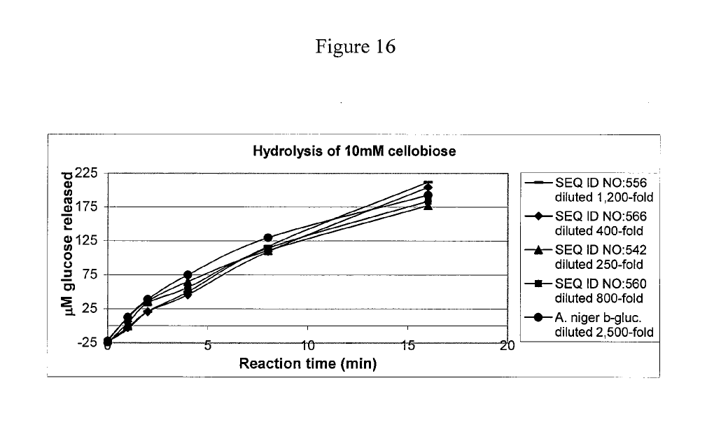

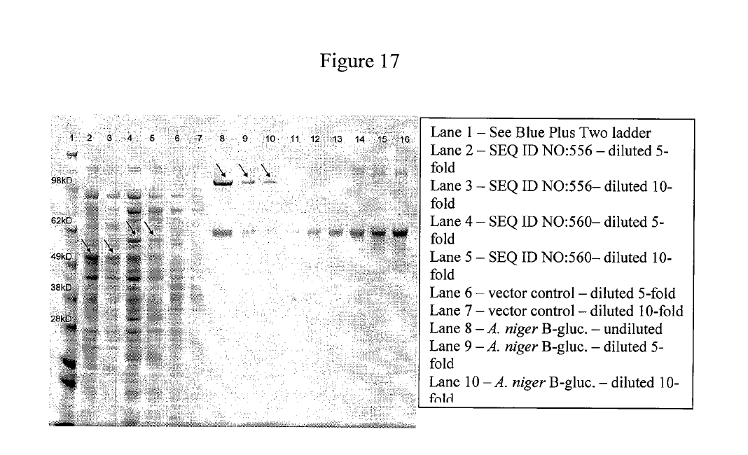

Megazyme beta-glucosidase (pure protein prep) was also evaluated in these experiments although direct comparison with exemplary beta-glucosidases of the invention could not be made given that enzymes of the invention used in this study were not purified. Results of this experiment are shown in FIG. 16 (cellobiose digestion) and FIG. 17 (PAGE electrophoresis).

FIG. 16: illustrates data of the initial rate kinetics with enzyme dilutions selected empirically for each tested beta-glucosidase enzyme of this invention.

FIG. 17: illustrates a PAGE electrophoresis with the exemplary SEQ ID NO:556, SEQ ID NO:560 of this invention, and A. niger beta-glucosidase. Equivalent of 1 μl of each sample was loaded in each lane. Arrows indicate protein bands corresponding to beta-glucosidase enzymes in each sample.

The following can be concluded based on the results obtained in this experiment:

(1) SEQ ID NO:556 and SEQ ID NO:560 performed the best of all tested beta-glucosidase enzymes of this invention (had initial rates at highest enzyme dilutions comparable to rates obtained with other enzymes used at lower dilutions);

(2) Based on a semi-quantitative analysis shown here, SEQ ID NO:556 and SEQ ID NO:560 had very comparable specific activities. According to PAGE electrophoresis data shown for these two enzymes, similar amount of each of the enzymes was present in protein preps used in the reaction. When gel shown on FIG. 17 was subjected to densitometry scan it was determined that approx. 1 mg/ml SEQ ID NO:556 enzyme and 0.7 mg/ml SEQ ID NO:560 enzyme were present in the corresponding protein preps diluted 10-fold for gel loading (1.5-fold more beta-glucosidase protein was present in SEQ ID NO:556 prep than in SEQ ID NO:560 prep); Since SEQ ID NO:556 was diluted 1.5-fold higher than SEQ ID NO:560 in cellobiose digestion reaction to achieve comparable reaction rates (1,200-fold vs. 800-fold, 25 μl volume used for each enzyme in 50 μl reaction volume), specific activities for these two enzymes appear very similar. BSA standard at concentrations listed in FIG. 16 was used for calibration in densitometry scan.

(3) Amount of beta-glucosidase present in Megazyme pure protein prep was estimated to approx. 1 mg/ml; Although this enzyme was diluted significantly higher to achieve comparable cellobiose digestion rates (2,500-fold vs. 1,200- or 800-fold for SEQ ID NO:556 and SEQ ID NO:560, respectively), its relative specific activity could not be compared to SEQ ID NO:556 and SEQ ID NO:560 since protein preps of very different purity were used in cellobiose digestions;

(4) Similar semi-quantitative determination of specific activity could not be done for SEQ ID NO:566, SEQ ID NO:542, and SEQ ID NO:548 because relevant protein bands could not be identified on PAGE gel due to low purity of protein preparations available for these enzymes. This is in correlation with significantly higher amount of material (lower protein prep dilutions) required for these enzymes to achieve rates comparable to SEQ ID NO:556 and SEQ ID NO:560.

(3) Residual Activity of Selected Beta-Glucosidases of the Invention in 4 Hour Cellobiose Digestion at High Temperatures.

Semi-pure protein preparations from subclone fermentations were diluted to achieve comparable reaction rates as discussed in the previous section: SEQ ID NO:556—1,200-fold, SEQ ID NO:566—400-fold, SEQ ID NO:542—250-fold, SEQ ID NO:548—300-fold, SEQ ID NO:560—800-fold. Megazyme A. niger beta-glucosidase was diluted 2,500-fold. 10 mM cellobiose was used as substrate and digestion reactions were run at 60° C., 70° C. and 80° C. for 4 h with time points taken at each hour. Reaction volume was 50 μl (substrate and enzyme present at 1:1 ratio).

The following was concluded based on the results obtained in this experiment:

(1) SEQ ID NO:556 and SEQ ID NO:560 remained active at all temperatures over 4 h reaction time;

(2) At the end of 4 h reaction, there was no significant difference in activity of these two enzymes at 60° C., 70° C. or 80° C. (comparable amounts of glucose were released at all temperatures);

(3) Based on the amount of glucose released over time, it appears that >60% of each enzyme remained active at 4 h. This finding indirectly suggests that the two enzymes are stable at higher temperatures and as such, could be suitable candidates to include in a 4-enzyme cocktail;

(4) SEQ ID NO:566 and SEQ ID NO:542 showed significant loss of activity at 70° C. and 80° C.; SEQ ID NO:548 retained about 50% activity at 70° C. and was inactive at 80° C.; (5) Loss in activity >75% was observed for Megazyme beta-glucosidase at 70° C. and the enzyme was inactive at 80° C.

Summary:

Ten exemplary beta-glucosidase of the invention were evaluated in several experiments discussed here and when practical, compared to commercial prep of beta-glucosidase from A. niger purchased from Megazyme. Exemplary enzymes of the invention SEQ ID NO:556 and SEQ ID NO:560 exhibited high reactivity at temperatures up to 90° C. and retained more than 60% activity at pH 5 in 4 hour reaction at 80° C. These two enzymes of the invention outperformed Megazyme commercial beta-glucosidase at 70° C. and 80° C. under test conditions and are considered as top candidates for a 4-enzyme cocktail at this time. Specific activity of beta-glucosidase enzymes of the invention could not be determined accurately due to low purity of protein preparations used in these experiments. Product inhibition is presently being studied with all enzymes evaluated in experiments discussed here.

Similar characterization can be performed with the other enzymes of the invention, e.g., the remaining four beta-glucosidases identified as top-performers based on data obtained on surrogate substrate (pNP-B-Glucopyranoside). Other enzymes of the invention can be subjected to characterization on cellobiose at different pH and temperatures in order to identify the best enzymes available for 4-enzyme cocktail. Top performing enzymes can be subjected to protein purification in order to determine specific activity more accurately. The best performing enzymes, which will be selected based on all criteria discussed here, can be incorporated in 4-enzyme cocktails and evaluated on pretreated bagasse in combinatorial screens.

### Example 16

### Characterization of Beta-Glucosidases of this Invention

The main objectives of these studies were: (1) Identify β-glucosidase enzymes of this invention for use in a four enzyme cocktail; and, (2) Identify several enzymes of this invention which could be used in a combinatorial assay.

Sixty seven beta-glucosidases were partially characterized. Based on the specific activity, pH and temperature profiles, 36 of these enzymes were selected for further evaluation in a four-step selection process which included the following:

(1) determination of activity on cellobiose and on fluorescent substrate 4-methylumbelliferyl beta-D-glucopyranoside (4-MU-GP) at pH5 and 7 and T=37-90 C;

(2) expression evaluation;

(3) determination of residual activity at T=60-80 C, pH5;

(4) determination of product inhibition. Commercial A. niger beta-glucosidase was used as benchmark in all experiments although direct comparisons would be difficult due to a significant difference in the quality of protein preparations which were available (lyophilized bacterial lysates vs. >90% pure protein in commercial prep). When cellobiose was used as substrate, enzyme activity was determined by glucose oxidase assay.

Methods/Results:

(1) Activity of Beta-Glucosidases at pH 5 and pH 7 and at Temperatures 37-90° C.

Semi-pure lyophilized protein preparations from subclone fermentations were reconstituted in 50% glycerol to obtain 25 mg/ml total protein concentration. Protein preps were subsequently diluted to 0.5 mg/mL and used in the reactions which were run for 1 hour at pH 5 and pH 7 and at 37, 40, 50, 60, 70, 80, and 90° C. in the presence of 10 mM cellobiose substrate (reaction volume was 50 μl; substrate and enzyme present at 1:1 ratio).

Most of the 36 enzymes of the invention which were tested exhibited pH 5 optimum. Seventeen enzymes were active at ≧50° C., including the exemplary enzymes of this invention SEQ ID NO:556, SEQ ID NO:540, SEQ ID NO:546, SEQ ID NO:566, SEQ ID NO:550, SEQ ID NO:530, SEQ ID NO:542, SEQ ID NO:554, SEQ ID NO:548, SEQ ID NO:564, SEQ ID NO:526, SEQ ID NO:560, SEQ ID NO:592, SEQ ID NO:586, SEQ ID NO:588, SEQ ID NO:578 and SEQ ID NO:558 (Table 1). Fourteen of these enzymes were active at ≧60° C.: SEQ ID NO:556, SEQ ID NO:540, SEQ ID NO:546, SEQ ID NO:566, SEQ ID NO:542, SEQ ID NO:554, SEQ ID NO:548, SEQ ID NO:564, SEQ ID NO:526, SEQ ID NO:560, SEQ ID NO:592, SEQ ID NO:588, SEQ ID NO:578 and SEQ ID NO:558. Thirteen of 36 enzymes had low or no apparent activity.

(2) Expression of Beta-Glucosidases and thorough Activity Characterization.

Equivalent of 0.5 mg/mL total protein concentration of semi-pure lyophilized protein preparations was loaded on the SDS-PAGE to determine protein expression and purity. Invitrogen's 4-12% NU-PAGE™ gels were used and electrophoresis was performed in 2× MES buffer at 200V for 50 min. Gels were processed and protein concentration was estimated by densitometry. For thorough activity testing, 0.5 mg/mL of semi-pure lyophilized protein preparations (bacterial cell lysates) were incubated at pH5 for 30 min with 10 mM cellobiose (T=37° C. and 50° C.) and with 1 mM 4-MU-GP (T=37° C.). Commercial A. niger beta-glucosidase (loaded at 0.4 cg/mL) was used as benchmark. Since this preparation contains >90% pure protein, enzyme loading was adjusted to obtain fluorescence which will remain within linear range of the glucose standard curve in the detection assay. Enzyme activity was determined and expressed as μM released glucose (cellobiose substrate) or as U/mL (4-MU-GP substrate).

Ten of seventeen enzymes of the invention which were active at ≧50° C. were also expressed at >3% purity. These enzymes showed range of activities on cellobiose and on 4-MU-GP. Expression and activity results are summarized in Table 1:

TABLE 1
Expression and activity of 17 selected beta-glucosidases.

| | | beta-glucosidase activity | | |
|---|---|---|---|---|
| | | 37° C. | | 50° C. |
| Enzyme | % purity | μM glucose | U/ml | μM glucose |
| SEQ ID NO: 556 | <3 | 358 | 13 | 323 |
| SEQ ID NO: 540 | <3 | 310 | 44 | 441 |
| SEQ ID NO: 546 | <3 | 450 | 5 | 520 |
| SEQ ID NO: 566 | 4 | 453 | 3 | 332 |
| SEQ ID NO: 550 | 8 | 294 | 1 | 227 |
| SEQ ID NO: 530 | 8 | 277 | 6 | 345 |
| SEQ ID NO: 542 | <3 | 412 | 2 | 333 |
| SEQ ID NO: 554 | 11 | 362 | 2 | 433 |
| SEQ ID NO: 548 | <5 | 461 | 3 | 423 |
| SEQ ID NO: 564 | <5 | 604 | 54 | 515 |
| SEQ ID NO: 526 | 5 | 423 | 4 | 457 |
| SEQ ID NO: 560 | <3 | 242 | 8 | 164 |
| SEQ ID NO: 592 | 7 | 158 | 24 | 174 |
| SEQ ID NO: 586 | 11 | 295 | 9 | 504 |
| SEQ ID NO: 588 | 8 | 273 | 13 | 260 |
| SEQ ID NO: 578 | 8 | 232 | 2 | 440 |
| SEQ ID NO: 558 | 35 | 488 | 10 | 338 |
| Megazyme b-gluc. | >90 | 407 | 24 | 471 |

Based on results summarized in Table 1, the following 8 enzymes were selected for expression optimization and large scale protein production: SEQ ID NO:556, SEQ ID NO:566, SEQ ID NO:530, SEQ ID NO:548, SEQ ID NO:564, SEQ ID NO:560, SEQ ID NO:586, and SEQ ID NO:558.

(3) Residual Activity After 0-4 h at pH 5 and T=60-80° C.

The semi-pure lyophilized preparations (bacterial cell lysates) of 17 enzymes listed in Table 1 (0.25 mg/mL total protein) and commercial A. niger beta-glucosidase (1.0 ug/mL of pure protein, benchmark) were incubated for 0-4 h at 60, 70 and 80° C. at pH 5 (1 mL reaction volume, 200 rpm mixing). Aliquots were removed at every hour and assayed for residual enzyme activity on cellobiose (10 mM, 1 h reaction time, pH5, 60° C.) and on 4-MU-GP (1 mM, 20 min reaction time, pH5, RT).

Results are summarized in Table 2 and FIG. 1. Seven of 17 tested enzymes retained residual activity at ≧60 C (SEQ ID NO:556, SEQ ID NO:560, SEQ ID NO:566, SEQ ID NO:530, SEQ ID NO:548, SEQ ID NO:564 and SEQ ID NO:554). Six of these enzymes remained 90% active after 4 h at 60° C. and SEQ ID NO:554 remained 25% active after 2 h at 60° C. SEQ ID NO:556 remained 90% active after 4 h incubation at 80° C. and outperformed commercial benchmark (A. niger beta-glucosidase). The benchmark remained active at 60° C., but after one hour at 70° C., it retained less than 20% activity and after one hour at 80° C., it had no activity.

TABLE 2
Residual beta-glucosidase activity after 4 h at 60-80° C.

| Temperature | approximately 90% residual activity after 4 h |
|---|---|
| 60° C. | SEQ ID NO: 566, SEQ ID NO: 530, SEQ ID NO: 548, |
| | SEQ ID NO: 564, SEQ ID NO: 560, SEQ ID NO: 556 |
| 70° C. | SEQ ID NO: 560, SEQ ID NO: 556 |
| 80° C. | SEQ ID NO: 556 |

(4) Product Inhibition in the Presence of 0-500 mM Glucose.

Digestion reactions containing 5 mM cellobiose and 0.5 mg/mL total protein of semi-pure lyophilized enzyme preparations and A. niger beta-glucosidase (0.025 mg/mL, benchmark) were dosed with 0, 25, 50, 100, 200, 300, 400 and 500 mM glucose (50□L total reaction volume) and incubated for 1 hat 60° C. at pH 5. Reactions were terminated by adding equal volume of 50 mM Na₂CO₃ buffer pH 10. Amount of cellobiose substrate left after 1 h digestion and amount of glucose present in each sample were analyzed by HPLC.

Results are summarized in Table 3, below. Three of the 13 enzymes of this invention which were tested, SEQ ID NO:566, SEQ ID NO:548 and SEQ ID NO:564, outperformed commercial benchmark and remained active in the presence of ≧300 mM glucose. SEQ ID NO:566 and SEQ ID NO:564 showed least product inhibition and remained active in the presence of ≧400 nM glucose. In a commercial process which would assume hydrolysis of Brazilian bagasse (approximately 50% cellulose content) at 10% solids and 50% enzymatic conversion, approx. 139 mM glucose (25 g/L) would be generated from 1 kg of starting material. Eight enzymes of the invention listed in Table 3 (SEQ ID NO:556, SEQ ID NO:540, SEQ ID NO:566, SEQ ID NO:530, SEQ ID NO:542, SEQ ID NO:548, SEQ ID NO:564, and SEQ ID NO:592) and Megazyme A. niger beta-glucosidase are expected to remain active under those conditions. However, in a process which would require ≧20% solids, commercial benchmark would be inhibited but SEQ ID NO:566, SEQ ID NO:548 and SEQ ID NO:564 should remain active.

TABLE 3
Table 3. Product inhibition in the presence of glucose.

| Enzyme | Product inhibition (mM glucose) |
|---|---|
| SEQ ID NO: 556 | 200 (starts at 25) |
| SEQ ID NO: 540 | 300 (starts at 50) |
| SEQ ID NO: 546 | 25 |
| SEQ ID NO: 566 | >400 (starts at 25) |
| SEQ ID NO: 550 | NT |
| SEQ ID NO: 530 | 200 (starts at 50) |
| SEQ ID NO: 542 | 200 (starts at 25) |
| SEQ ID NO: 554 | 100 (starts at 50) |
| SEQ ID NO: 548 | 400 (starts at 100) |
| SEQ ID NO: 564 | 500 (starts at 25 |
| SEQ ID NO: 526 | 25 |
| SEQ ID NO: 560 | — |
| SEQ ID NO: 592 | 200 (starts at 25) |
| SEQ ID NO: 586 | — |
| SEQ ID NO: 588 | 100 (starts at 25) |
| SEQ ID NO: 578 | NT |
| SEQ ID NO: 558 | 50 (starts at 25) |
| Megazyme beta-gluc. | 300 (starts at 50) |

NT, not tested.

Summary:

Several beta-glucosidases of this invention were identified using the exemplary four-step selection process described in this study. These enzymes of this invention can be used in combinatorial screening and in enzyme cocktails, e.g. 4-enzyme cocktails. Exemplary enzymes of this invention candidates identified in these studies based on individual selection criteria are listed in Table 4, below:

TABLE 4
Best performing enzymes based on individual selection criteria.

| Selection criteria | Best performers |
|---|---|
| Activity at 37-90° C./ | SEQ ID NO: 556, SEQ ID NO: 566, SEQ |
| pH5 and expression | ID NO: 530, SEQ ID NO: 554, SEQ ID NO: 526, |
| | SEQ ID NO: 560, SEQ ID NO: 586, SEQ ID |
| | NO: 588, SEQ ID NO: 558 |
| Residual activity at | SEQ ID NO: 556, SEQ ID NO: 566, SEQ |
| 60-80° C./pH5 | ID NO: 530, SEQ ID NO: 554, SEQ ID NO: 548, |
| | SEQ ID NO: 564, SEQ ID NO: 560, |
| Level of product | SEQ ID NO: 566, SEQ ID NO: 548, SEQ |
| inhibition | ID NO: 564 |

### Example 17

### Enzyme-Coupled Cellulase Discovery Screen of this Invention

The example describes an exemplary enzyme-coupled cellulase discovery screen of this invention, which in this particular example uses the exemplary enzyme of the invention SEQ ID NO:580, a β-glucosidase. The activity per milligram of protein will vary from prep to prep, depending on success of purification. To better standardize the discovery screen, the activity of a particular batch of this enzyme is now described in U/mL. This protocol describes how to do this.

Unit Definition

For this example, the unit definition designates that one unit of SEQ ID NO:580 enzyme will liberate 1.0 μmol of 4-methylumbelliferone from MU-glucopyranoside substrate per minute at pH 7.5 at room temperature (approximately 22° C.).

### Protocol

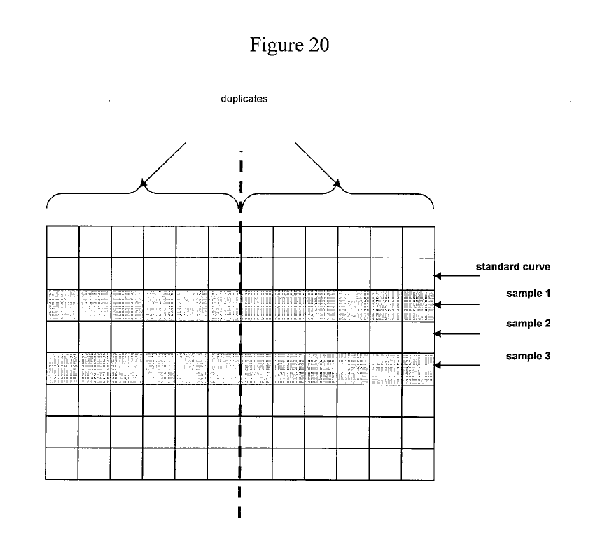

1. Use a black 96-well plate. Plan to position all samples for fast, simultaneous addition of samples and to provide the shortest interval for a kinetic read. FIG. 20 illustrates an example arrangement for three sample preps, where the reading range is B1-E12.

2. Make dilutions for a standard curve. Dilute 4-MU in 50 mM sodium phosphate buffer pH 7.5. Make 300 μL of each dilution: 0, 1, 2, 4, 8, and 16 μM.

3. Make dilutions of enzyme samples. For each enzyme sample to be tested, make successive 2-fold dilutions, typically starting at 1/500 (i.e., 1/500, 1/1000, 1/2000, 1/4000, 1/8000, and 1/16000). Make 150 μL of each dilution using 50 mM sodium phosphate pH 7.5 buffer as diluent.

4. Prepare 2× substrate. Prepare a 2 mM solution of MU-β-glucopyranoside substrate by diluting the stock substrate in 50 mM sodium phosphate buffer pH 7.5. Make about 1 mL of this solution for each enzyme sample to be tested. Place the solution into a pipetting basin.

5. Load the standard curve. Load, in duplicate at 100 μL/well onto black 96-well plate.

6. Load enzymes. Load enzymes in duplicate at 50 μL per well of each enzyme dilution.

7. Set up the SPECTRAMAX™. Set for kinetic read at room temperature with Ex/Em at 360/465 nm. Take readings at 30-second intervals for a total of 5 minutes. Set PMT sensitivity to medium. Make sure the standard curve samples are included in the kinetic read so that they are read under the same conditions.

8. Add substrate and start kinetic read. Put plate on SPECTRAMAX™ tray, lid off. Quickly add 50 μL of 2× substrate solution to the wells containing enzyme sample. Use a multichannel pipet for fast, simultaneous addition and mixing of samples. Immediately start kinetic read; save data. Make sure Vmax is calculated by the program in mRFU per minute.

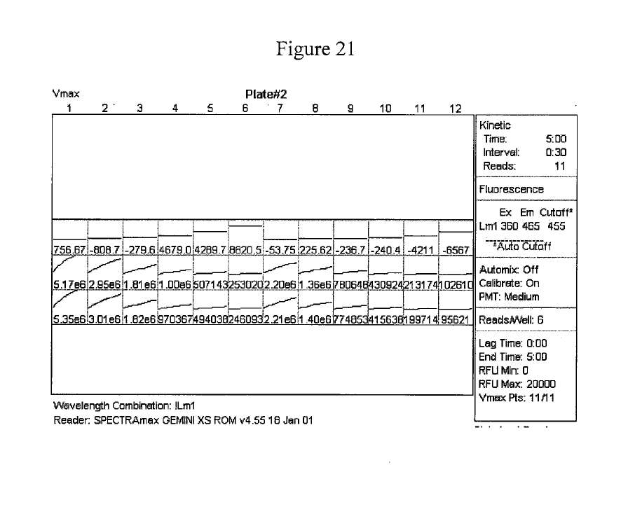

See FIG. 21, a table summarizing SPECTRAMAX™ data for this cellulase enzyme activity study (liberating 4-methylumbelliferone from MU-glucopyranoside) as “plate 2”.

### Calculations

### Standard Curve

1. Use the first kinetic read to gather data for standard curve (since the standard curve is just an endpoint measurement). First convert the μM values to μmol; for example, 100 μL of 25 μM=0.0025 μmol. For each point on the standard, calculate average and standard deviation for the duplicate samples, then subtract the background from the average RFU values.

2. Generate a scatter plot using the background-subtracted average values, with μmol 4-MU on the x-axis and RFU on the y-axis.

3. Use linear regression to generate the line function relating RFU to μmol 4-MU present. (Since background was subtracted, force y-intercept to 0.) Note: if the R-value is below 0.998, try omitting the data point representing the highest concentration on the standard curve. In MS EXCEL™, format the line function in scientific notation with two (2) decimal points.

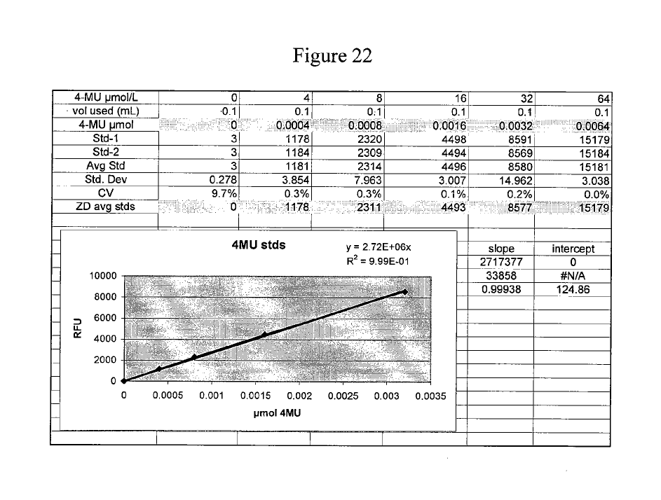

See FIG. 22, a table summarizing kinetic activity data for this cellulase enzyme activity study (liberating 4-methylumbelliferone from MU-glucopyranoside),

### Enzyme Activity

1. For each measurement of β-gluc activity, use the dilution that best fits the sensitivity range of the standard curve. Within this dilution, use only the data points that fall within the linear region of the kinetic to generate the Vmax. (Vmax is automatically calculated in the SPECTRAMAX™ software (in mRFU/min).) Calculate the average and standard deviation for each pair of duplicates. Then divide the average mRFU/min by 1000 to get RFU/min.

2. Use the line function to translate this value to units of β-gluc activity in μmol/min (RFU/min divided by slope), then multiply by the dilution factor to translate to Vmax in mol/min.

3. Take the calculated units and multiply it by the dilution factor used, then divide by the volume added to the well (0.05 mL) to get activity in U/mL.

4. The following shows how all calculations can be set up in EXCEL™, using SEQ ID NO:580 as an example, given a standard curve slope of 2,717,377:

PREP

| | | | | | | |
|---|---|---|---|---|---|---|
| #pts based on | 11 | 11 | 11 | 11 | 11 | 11 |
| mRFU/min 1 | 2202734 | 1359733 | 780648 | 430924 | 213174 | 102610 |
| mRFU/min 2 | 2211504 | 1399352 | 774853 | 415638 | 199714 | 95621 |
| avg mRFU/min | 2207119 | 1379542 | 777750 | 423281 | 206444 | 99115 |
| std dev | 6201 | 28015 | 4097 | 10809 | 9518 | 4942 |
| CV | 0.28% | 2.03% | 0.53% | 2.55% | 4.61% | 4.99% |
| RFU/min | 2207 | 1380 | 778 | 423 | 206 | 99 |
| dilution | 500 | 1000 | 2000 | 4000 | 8000 | 16000 |
| activity (μmole/min) | 0.406112122 | 0.507674328 | 0.572427335 | 0.623072647 | 0.60777468 | 0.583594115 |
| vol added (ml) | 0.05 | 0.05 | 0.05 | 0.05 | 0.05 | 0.05 |
| U/mL | 8.1 | 10.2 | 11.4 | 12.5 | 12.2 | 11.7 |

The standard curve and the kinetics should be measured on the SpectraMax together in the same read so that the sensitivity between standard and sample is matched.
If 4-MU standards are at all suspect, make fresh dilutions from a good stock.
Use the same buffer pH for the standard curve and the sample measurements. The fluorescence of 4-MU is highly dependent on pH.

### Reagents

### 4-MU

4-Methylumbelliferone (Sigma M1381, FW 176.2): Make a 50 mM stock solution in DMSO and store at −20° C. protected from light.

### MU-β-Glucopyranoside

4-Methylumbelliferyl β-D-glucopyranoside (Sigma M3633, FW 338.3): Make a stock solution at 50 mM in DMSO. Store aliquots at −20° C. protected from light.

### Example 18

### Construction of CBM Chimeric Enzymes

In one embodiment, the invention provides chimeric (e.g., multidomain recombinant) proteins comprising a first domain comprising a signal sequence and/or a carbohydrate binding domain (CBM) of the invention and at least a second domain. The protein can be a fusion protein. The second domain can comprise an enzyme. The protein can be a non-enzyme, e.g., the chimeric protein can comprise a signal sequence and/or a CBM of the invention and a structural protein. This example describes an exemplary protocol for making CBM-comprising polypeptides of this invention.

CBM Swapping Library Construction

A GENEREASSEMBLY™ variant library (1080 variants) was constructed using GENEREASSEMBLY™ technology (Verenium Corporation, San Diego, Calif.), using 6 CBHI catalytic domains (see Table 1, below), 30 CBMs derived from fungal and bacterial GHs (see Table 2, below), and 6 natural linkers extracted from GH genes (see Table 3, below).

The library of DNA fragments representing the CBH variants was cloned into an expression vector for Aspergillus niger containing:
a) a marker gene giving antibiotic resistance to the transformed A. niger host,
b) two regions of DNA with homology to the A. niger genome, to direct stable integration of the expression cassette into the genome by homologous recombination, one of which also serves as a transcriptional terminator,
c) a promoter to drive expression of the CBH, and d) an E. coli replicon containing a marker gene conferring antibiotic resistance to an E. coli host.

The vector used for screening the CBH variants (pDC-A1) was a reconstruction of the vector pGBFin-5 (described, e.g., in U.S. Pat. No. 7,220,542), that was remade to reduce the total size of the vector. The 2.1 kb 3′ Gla region of pGBFin-5 was reduced to 0.54 kb, the gpd promoter remained the same, but the 2.24 kb amdS sequence was replaced by the 1.02 kb hygB gene encoding hygromycin phosphotransferase. The 2.0 kb glucoamylase promoter gla, was reduced to a 1.15 kb fragment representing the 3′ end of the original sequence. The 2.3 kb 3′ Gla region of pGBFin-5 was also reduced to a 1.1 kb fragment representing the 5′ end of the original sequence. The E. coli replicon for pDC-A1 was taken from pUC18. Overall, the original 12.5 kb pGBFin-5 expression vector (without an insert of a gene intended to be expressed) was reduced to a 7.2 kb vector (no insert).

After ligation of pools of CBH variant ORF DNA to the vector, the ligation mixture was used to transform chemically competent E. coli Stbl2. Individual E. coli transformants were picked into 96-well plates and grown in liquid culture in 200 μl LB plus ampicillin (100 μg/ml) per well overnight at 30° C. The cells were then used to generate template for sequencing reactions by colony PCR. The sequence data from the library of clones was analyzed to identify unique variants of CBH. The E. coli transformants containing the selected variants were then rearrayed in 96-well format and used to prepare linear DNA of the entire expression cassette (the contents of pDC-A1 with the exception of the E. coli replicon) by PCR, using primers hybridizing to the ends of the 3′ and 3″ Gla regions. Approximately 1 μg of PCR product from each clone was then used to transform A. niger CBS153.88 protoplasts in a PEG-mediated transformation in one well of a 96-well plate (i.e. one clone per well). Transformants were selected on regeneration agar (200 μl per well of PDA plus sucrose at 340 g/l and hygromycin at 200 μg/ml) in the same 96-well format. After 7 days incubation at 30° C., transformants were replicated to 96-well plates containing PDA plus hygromycin (200 μg/ml) using a pintool. Following incubation at 30° C. for a further 7 days, spores from each well were used to inoculate 200 μl liquid media per well of a 96-well plate. The plates were incubated at 30° C. for 7 days, and the supernatant from each well, containing the secreted CBH variant, was recovered.

The media used to grow the Aspergillus transformed with expression constructs containing the variants had the following composition: NaNO₃, 3.0 g/l; KCl, 0.26 g/l; KH₂PO₄, 0.76 g/l; 4M KOH, 0.56 ml/l; D-Glucose, 5.0 g/l; Casamino Acids, 0.5 g/l; Trace Element Solution 0.5 ml/l; Vitamin Solution 5 ml/l; Penicillin-Streptomycin Solution (10,000 U/ml and 10,000 μg/ml respectively) 5.0 ml/l; Maltose, 66.0 g/l; Soytone, 26.4 g/l; (NH₄)₂SO₄, 6.6 g/l; NaH₂PO₄.H₂O, 0.44 g/l; MgSO₄.7H₂O, 0.44 g/l; Arginine, 0.44 g/l; Tween-80, 0.035 ml/l; Pleuronic Acid Antifoam, 0.0088 ml/l; MES, 18.0 g/l. The Trace Element Solution had the following composition in 100 ml: ZnSO₄.7H₂O, 2.2 g; H₃BO₃, 1.1 g; FeSO₄.7H₂O, 0.5 g; CoCl₂.6H₂O, 0.17 g; CuSO₄.5H₂O, 0.16; MnCl₂.4H₂O, 0.5 g/l; NaMoO₄.2H₂O, 0.15 g/l; EDTA, 5 g/l. The Vitamin Solution had the following composition in 500 ml: Riboflavin, 100 mg; Thiamine.HCl, 100 mg; Nicotinamide, 100 mg; Pyridoxine.HCl, 50 mg; Panthotenic Acid, 10 mg; Biotin 0.2 mg.

Primary Assay Screen Protocol

Ground (60-mesh) bagasse substrate, called pBG10C, is diluted to a final of 0.2% cellulose in 50 mM acetate buffer pH 5. 200 μL/well of this are added into a 96-well “substrate” plate. In a 96 well “cocktail” plate 10× enzyme cocktails are made containing the exemplary enzymes of the invention SEQ ID NO:90 (EG), SEQ ID NO:358 (CBHII), and CBHI CBM supernatant grown in A. niger. The final doses are 4 mg EG/g cellulose, 2 mg CBHII/g cellulose, and variable CBHI. To initiate the reaction 22 μL of enzyme cocktail is added to 200 μL of buffered substrate. We perform this digest at 37° C. Timepoints are taken from 0 to 48 hours. Timepoints are taken by transferring the reaction into a 384 well “stop” plate (200 mM Na carbonate buffer pH10). Next an overnight β-glucosidase digest is run on the “stop” plates to breakdown cellobiose into glucose.

A Glucose Oxidase (GO) assay is then run to measure amount of total glucose generated. CBM mutants were considered active if they showed a GO signal 2× the average of the negative control (vector only). This gave 159 active clones.

TABLE 1
Six Catalytic Domains (CD) Selected For
GENEREASSEMBLY ™ Library

| | CBHI Parental sequence | | | |
|---|---|---|---|---|
| CD | (which includes | GH | Size | |
| Identifier | Catalytic Domain) | Family | (kD) | CBM Location |
| 1 | SEQ ID NO: 360 | 7 | 51.3 | C-terminus |
| 2 | SEQ ID NO: 34 | 7 | 52.6 | C-terminus |
| 3 | SEQ ID NO: 371 | 7 | 54.3 | C-terminus |
| 4 | SEQ ID NO: 606 | 7 | 56.8 | C-terminus |
| 5 | SEQ ID NO: 608 | 7 | 48.3 | Whole sequence |
| 6 | SEQ ID NO: 610 | 7 | 47.9 | Whole sequence |

TABLE 2
Thirty CBMs Selected For GENEREASSEMBLY ™ Library

| CBM | CBM | Parental sequence (which | GH | |
|---|---|---|---|---|
| Identifier | Family | includes CBM) | Family | CBM Location |
| 1 | 3a | Genbank Accession No. | | |
| | | ZP_01575466 | | |
| | | (C. celluolyticum | | |
| | | CipC) | | |
| 2 | 3a | Genbank Accession No. | | |
| | | L08665 | | |
| | | (C. thermocellum | | |
| | | CipA) | | |
| 3 | 3a | Genbank Accession No. | | |
| | | P38058 (C. cellulovorans | | |
| | | CbpA) | | |
| 4 | 3a | Genbank Accession No. | | |
| | | AB004845 (C. josui | | |
| | | CipA) | | |
| 5 | 2a | SEQ ID NO: 468 | 8 | First CBM |
| | | | | (AA 466-570) |
| 6 | 2a | SEQ ID NO: 468 | 8 | Second CBM |
| | | | | (AA 646-746) |
| 7 | 2a | SEQ ID NO: 464 | 5 | N-terminus |
| 8 | 2a | SEQ ID NO: 6 | 6 | C-terminus |
| 9 | 2a | SEQ ID NO: 450 | 5 | N-terminus |
| 10 | 2a | SEQ ID NO: 140 | 48 | N-terminus |
| 11 | 2a | SEQ ID NO: 470 | 5 | C-terminus |
| 12 | 2a | SEQ ID NO: 614 | 12 | |
| 13 | 2a | SEQ ID NO: 4 | 6 | N-terminus |
| 14 | 2a | SEQ ID NO: 612 | | N-terminus |
| 15 | 17_28 | SEQ ID NO: 8 | 5 | internal |
| 16 | 17_28 | SEQ ID NO: 22 | 5 | internal |
| 17 | 17_28 | SEQ ID NO: 10 | 5 | internal |
| 18 | 17_28 | SEQ ID NO: 430 | 5 | internal |
| 19 | 1 | SEQ ID NO: 360 | 7 | C-terminus |
| 20 | 1 | SEQ ID NO: 34 | 7 | C-terminus |
| 21 | 1 | SEQ ID NO: 371 | 7 | C-terminus |
| 22 | 1 | SEQ ID NO: 606 | 7 | C-terminus |
| 23 | 1 | SEQ ID NO: 616 | 7 | C-terminus |
| 24 | 1 | SEQ ID NO: 282 | 6 | N-terminus |
| 25 | 1 | SEQ ID NO: 358 | 6 | N-terminus |
| 26 | 1 | SEQ ID NO: 618 | 7 | C-terminus |
| 27 | 1 | SEQ ID NO: 32 | 7 | C-terminus |
| 28 | 1 | SEQ ID NO: 604 | 7 | C-terminus |
| 29 | 1 | SEQ ID NO: 620 | 6 | N-terminus |
| 30 | 1 | SEQ ID NO: 30 | 7 | C-terminus |

TABLE 3
Six linkers selected for GENEREASSEMBLY ™ library

| | Linker | | |
|---|---|---|---|
| | from | | |
| Linker | CBM | Parental sequence | |
| Identifier | Family | containing linker | Linker AA Sequence |
| 1 | CBM1 | SEQ ID NO: 360 | SGGSSTGGSSTTTASGTTTTKASSTSTSSTS |
| | | | TGTGV (SEQ ID NO: xxxx) |
| 2 | CBM1 | SEQ ID NO: 371 | SGTGGNNPDPEEPEEPEEPV GT (SEQ ID |
| | | | NO: xxxx) |
| 3 | CBM1 | SEQ ID NO: 606 | NSGSTGGGNGSGSTTTTKGSTTTTKAPTTT |
| | | | TTTSKATTTTAASGGNGGG (SEQ ID |
| | | | NO: xxxx) |
| 4 | CBM2a | Thermobifida fusca | TNPNPNPNPTPTPTPTPTPPPGSS (SEQ ID |
| | | GH6 (Genbank | NO: xxxx) |
| | | YP_289135) | |
| 5 | CBM2a | Saccharophagus | SGSSSSSSSSSSSSSSSSSSSSSTSSSSSSSSST |
| | | degradans | SSSSSSSGSSGT (SEQ ID NO: xxxx) |
| | | (Genbank | |
| | | YP_527744) | |
| 6 | CBM2a | Xylella fastidiosa | GGGASGGSGGGAGASSGSGAGGGSSGGA |
| | | (Genbank | GTGSGSGA (SEQ ID NO: xxxx) |
| | | NP_780034.1) | |

TABLE 4
159 active unique CBHI/CBM hybrids

| | Linker | |
|---|---|---|
| | from CBM | CBM |
| Hybrid | Family | Family |
| CD6 + LINK2 + CBM15 | 1 | 17 |
| CD6 + LINK3 + CBM27 | 1 | 1 |
| CD6 + LINK3 + CBM23 | 1 | 1 |
| CD6 + LINK3 + CBM25 | 1 | 1 |
| CD6 + LINK3 + CBM28 | 1 | 1 |
| CD6 + LINK3 + CBM10 | 1 | 2a |
| CD6 + LINK1 + CBM7 | 1 | 2a |
| CD6 + LINK1 + CBM23 | 1 | 1 |
| CD6 + LINK1 + CBM17 | 1 | 17 |
| CD6 + LINK1 + CBM28 | 1 | 1 |
| CD6 + LINK1 + CBM10 | 1 | 2a |
| CD6 + LINK4 + CBM6 | 2a | 2a |
| CD6 + LINK4 + CBM2 | 2a | 3a |
| CD6 + LINK4 + CBM10 | 2a | 2a |
| CD6 + LINK6 + CBM7 | 2a | 2a |
| CD6 + LINK6 + CBM25 | 2a | 1 |
| CD6 + LINK6 + CBM22 | 2a | 1 |
| CD6 + LINK6 + CBM26 | 2a | 1 |
| CD6 + LINK6 + CBM9 | 2a | 2a |
| CD6 + LINK5 + CBM9 | 2a | 2a |
| CD3 + LINK2 + CBM21 | 1 | 1 |
| CD3 + LINK3 + CBM11 | 1 | 2a |
| CD3 + LINK3 + CBM6 | 1 | 2a |
| CD3 + LINK3 + CBM7 | 1 | 2a |
| CD3 + LINK3 + CBM23 | 1 | 1 |
| CD3 + LINK3 + CBM20 | 1 | 1 |
| CD3 + LINK3 + CBM28 | 1 | 1 |
| CD3 + LINK3 + CBM10 | 1 | 2a |
| CD3 + LINK1 + CBM19 | 1 | 1 |
| CD3 + LINK6 + CBM2 | 2a | 3a |
| CD3 + LINK6 + CBM17 | 2a | 17 |
| CD3 + LINK6 + CBM28 | 2a | 1 |
| CD3 + LINK5 + CBM30 | 2a | 1 |
| CD3 + LINK5 + CBM12 | 2a | 2a |
| CD4 + LINK2 + CBM8 | 1 | 2a |
| CD4 + LINK3 + CBM6 | 1 | 2a |
| CD4 + LINK3 + CBM10 | 1 | 2a |
| CD4 + LINK1 + CBM6 | 1 | 2a |
| CD4 + LINK1 + CBM8 | 1 | 2a |
| CD4 + LINK1 + CBM7 | 1 | 2a |
| CD4 + LINK1 + CBM23 | 1 | 1 |
| CD4 + LINK1 + CBM21 | 1 | 1 |
| CD4 + LINK1 + CBM29 | 1 | 1 |
| CD4 + LINK1 + CBM28 | 1 | 1 |
| CD4 + LINK1 + CBM10 | 1 | 2a |
| CD4 + LINK4 + CBM21 | 2a | 1 |
| CD4 + LINK6 + CBM7 | 2a | 2a |
| CD4 + LINK6 + CBM27 | 2a | 1 |
| CD4 + LINK6 + CBM21 | 2a | 1 |
| CD4 + LINK6 + CBM15 | 2a | 17 |
| CD4 + LINK6 + CBM25 | 2a | 1 |
| CD4 + LINK6 + CBM26 | 2a | 1 |
| CD4 + LINK6 + CBM28 | 2a | 1 |
| CD4 + LINK6 + CBM10 | 2a | 2a |
| CD4 + LINK5 + CBM8 | 2a | 2a |
| CD4 + LINK5 + CBM7 | 2a | 2a |
| CD4 + LINK5 + CBM27 | 2a | 1 |
| CD4 + LINK5 + CBM21 | 2a | 1 |
| CD4 + LINK5 + CBM24 | 2a | 1 |
| CD2 + LINK3 + CBM6 | 1 | 2a |
| CD2 + LINK3 + CBM8 | 1 | 2a |
| CD2 + LINK3 + CBM7 | 1 | 2a |
| CD2 + LINK3 + CBM30 | 1 | 1 |
| CD2 + LINK3 + CBM23 | 1 | 1 |
| CD2 + LINK3 + CBM21 | 1 | 1 |
| CD2 + LINK3 + CBM15 | 1 | 17 |
| CD2 + LINK3 + CBM25 | 1 | 1 |
| CD2 + LINK3 + CBM29 | 1 | 1 |
| CD2 + LINK3 + CBM20 | 1 | 1 |
| CD2 + LINK3 + CBM28 | 1 | 1 |
| CD2 + LINK3 + CBM10 | 1 | 2a |
| CD2 + LINK3 + CBM12 | 1 | 2a |
| CD2 + LINK1 + CBM6 | 1 | 2a |
| CD2 + LINK1 + CBM7 | 1 | 2a |
| CD2 + LINK1 + CBM23 | 1 | 1 |
| CD2 + LINK1 + CBM21 | 1 | 1 |
| CD2 + LINK1 + CBM22 | 1 | 1 |
| CD2 + LINK1 + CBM10 | 1 | 2a |
| CD2 + LINK1 + CBM9 | 1 | 2a |
| CD2 + LINK1 + CBM12 | 1 | 2a |
| CD2 + LINK4 + CBM6 | 2a | 2a |
| CD2 + LINK4 + CBM10 | 2a | 2a |
| CD2 + LINK6 + CBM6 | 2a | 2a |
| CD2 + LINK6 + CBM7 | 2a | 2a |
| CD2 + LINK6 + CBM27 | 2a | 1 |
| CD2 + LINK6 + CBM30 | 2a | 1 |
| CD2 + LINK6 + CBM21 | 2a | 1 |
| CD2 + LINK6 + CBM29 | 2a | 1 |
| CD2 + LINK6 + CBM20 | 2a | 1 |
| CD2 + LINK6 + CBM10 | 2a | 2a |
| CD2 + LINK5 + CBM20 | 2a | 1 |
| CD2 + LINK5 + CBM19 | 2a | 1 |
| CD2 + LINK5 + CBM26 | 2a | 1 |
| CD2 + LINK5 + CBM28 | 2a | 1 |
| CD2 + LINK5 + CBM10 | 2a | 2a |
| CD2 + LINK5 + CBM12 | 2a | 2a |
| CD1 + LINK3 + CBM8 | 1 | 2a |
| CD1 + LINK3 + CBM30 | 1 | 1 |
| CD1 + LINK3 + CBM23 | 1 | 1 |
| CD1 + LINK3 + CBM21 | 1 | 1 |
| CD1 + LINK3 + CBM22 | 1 | 1 |
| CD1 + LINK3 + CBM20 | 1 | 1 |
| CD1 + LINK3 + CBM19 | 1 | 1 |
| CD1 + LINK3 + CBM26 | 1 | 1 |
| CD1 + LINK3 + CBM28 | 1 | 1 |
| CD1 + LINK3 + CBM9 | 1 | 2a |
| CD1 + LINK3 + CBM12 | 1 | 2a |
| CD1 + LINK1 + CBM6 | 1 | 2a |
| CD1 + LINK1 + CBM8 | 1 | 2a |
| CD1 + LINK1 + CBM7 | 1 | 2a |
| CD1 + LINK1 + CBM30 | 1 | 1 |
| CD1 + LINK1 + CBM23 | 1 | 1 |
| CD1 + LINK1 + CBM21 | 1 | 1 |
| CD1 + LINK1 + CBM24 | 1 | 1 |
| CD1 + LINK1 + CBM29 | 1 | 1 |
| CD1 + LINK1 + CBM19 | 1 | 1 |
| CD1 + LINK1 + CBM26 | 1 | 1 |
| CD1 + LINK1 + CBM28 | 1 | 1 |
| CD1 + LINK1 + CBM10 | 1 | 3a |
| CD1 + LINK1 + CBM12 | 1 | 2a |
| CD1 + LINK4 + CBM21 | 2a | 1 |
| CD1 + LINK6 + CBM7 | 2a | 2a |
| CD1 + LINK6 + CBM27 | 2a | 1 |
| CD1 + LINK6 + CBM30 | 2a | 1 |
| CD1 + LINK6 + CBM23 | 2a | 1 |
| CD1 + LINK6 + CBM21 | 2a | 1 |
| CD1 + LINK6 + CBM25 | 2a | 1 |
| CD1 + LINK6 + CBM19 | 2a | 1 |
| CD1 + LINK6 + CBM10 | 2a | 2a |
| CD1 + LINK6 + CBM9 | 2a | 2a |
| CD1 + LINK6 + CBM12 | 2a | 2a |
| CD1 + LINK5 + CBM11 | 2a | 2a |
| CD1 + LINK5 + CBM27 | 2a | 1 |
| CD1 + LINK5 + CBM21 | 2a | 1 |
| CD1 + LINK5 + CBM28 | 2a | 1 |
| CD1 + LINK5 + CBM10 | 2a | 2a |
| CD1 + LINK5 + CBM9 | 2a | 2a |
| CD1 + LINK5 + CBM12 | 2a | 2a |
| CD5 + LINK3 + CBM3 | 1 | 3a |
| CD5 + LINK3 + CBM19 | 1 | 1 |
| CD5 + LINK3 + CBM28 | 1 | 1 |
| CD5 + LINK3 + CBM10 | 1 | 2a |
| CD5 + LINK1 + CBM6 | 1 | 2a |
| CD5 + LINK1 + CBM30 | 1 | 1 |
| CD5 + LINK1 + CBM21 | 1 | 1 |
| CD5 + LINK1 + CBM25 | 1 | 1 |
| CD5 + LINK4 + CBM6 | 2a | 2a |
| CD5 + LINK6 + CBM5 | 2a | 2a |
| CD5 + LINK6 + CBM1 | 2a | 3a |
| CD5 + LINK6 + CBM27 | 2a | 1 |
| CD5 + LINK6 + CBM23 | 2a | 1 |
| CD5 + LINK6 + CBM22 | 2a | 1 |
| CD5 + LINK6 + CBM29 | 2a | 1 |
| CD5 + LINK6 + CBM19 | 2a | 1 |
| CD5 + LINK6 + CBM26 | 2a | 1 |
| CD5 + LINK5 + CBM16 | 2a | 17 |
| CD5 + LINK5 + 294EG3 | 2a | 17 |
| CD5 + LINK5 + CBM24 | 2a | 1 |
| CD5 + LINK5 + CBM22 | 2a | 1 |

### Example 19

### Saccharification of a Biomass

In one embodiment, the invention provides polypeptides having a lignocellulosic activity, including enzymes that convert soluble oligomers to fermentable monomeric sugars, for the saccharification of a biomass. In one aspect, an activity of a polypeptide of the invention comprises enzymatic hydrolysis of (to degrade) soluble cellooligsaccharides and arabinoxylan oligomers into monomer xylose, arabinose and glucose. In one aspect, exemplary enzymes of the invention are used in processes for the saccharification of cellulose or cellulose-comprising compositions, such as plant biomass, e.g., sugarcane bagasse, corn fiber or other plant waste material (such as a hay or straw, e.g., a rice straw or a wheat straw, or any the dry stalk of any cereal plant) or processing or agricultural byproduct. This example describes an exemplary saccharification process of the invention.

Saccharification Reaction Runs:

Method

250 dw mg of steam exploded bagasse was weighed into 10 mL glass crimp-top vials (5% solids). A volume of MES buffered minimal media (pH5.6) was added to each vial, depending on the enzyme loading, for a final substrate content of 5% solids. Enzymes cocktails were added to the bagasse mixture at 10 mg enzyme per gram solids, with an equal amount of each enzyme component (1:1:1). The total reaction volume is 5 mL (therefore 2.5 mg total enzyme/rxn). A 200 uL time 0 sample was removed from the reaction and frozen at −20° C. The vials were sealed and clamped and placed in a shaking 37° C. incubator. 200 uL samples were removed at 24, 48, and 90 hours. Samples were thawed, spun at 13,200 rpm for 5 minutes, and the supernatant was diluted 10 fold. Sugar composition of these reaction products were analyzed by HPLC against known standards. Conversion was calculated based on the theoretical cellulose value in the bagasse substrate.

| | all cocktails at a 1:1:1 ratio | | | | | |
|---|---|---|---|---|---|---|
| | enzyme | | | | | |
| | components | | | % conversion | | |
| Cocktail # | CBH1 | CBH2 | EG | 24 | 48 | 90 |
| G8 | SEQ ID NO: 360 | SEQ ID NO: 358 | SEQ ID NO: 90 | 29.0 | 43.2 | 52.1 |
| G8F-1 | SEQ ID NO: 360 | SEQ ID NO: 358 | SEQ ID NO: 502 | 30.4 | 41.3 | 49.0 |
| G8F-2 | SEQ ID NO: 360 | SEQ ID NO: 358 | SEQ ID NO: 500 | 31.6 | 42.9 | 50.7 |
| 4 | SEQ ID NO: 360 | SEQ ID NO: 282 | SEQ ID NO: 90 | 22.3 | 33.9 | 34.8 |
| 5 | SEQ ID NO: 360 | SEQ ID NO: 282 | SEQ ID NO: 502 | 27.5 | 40.2 | 42.7 |
| 6 | SEQ ID NO: 360 | SEQ ID NO: 282 | SEQ ID NO: 500 | 23.3 | 35.2 | 34.8 |
| 7 | SEQ ID NO: 360 | SEQ ID NO: 598 | SEQ ID NO: 90 | 27.9 | 40.5 | 50.1 |
| 8 | SEQ ID NO: 360 | SEQ ID NO: 598 | SEQ ID NO: 502 | 28.4 | 38.7 | 47.3 |
| 9 | SEQ ID | SEQ ID | SEQ ID | 29.7 | 39.6 | 46.7 |
| | NO: 360 | NO: 598 | NO: 500 | | | |

96-Well Plate Assay—Percent Bagasse Conversion:

Method

Steam exploded bagasse was resuspended in MES buffered minimal media (pH 5.6) at 0.4% cellulose. 200 ul of buffered substrate was added to each well in a 96-well plate. Enzyme cocktails were prepared at 0.432 mg enzyme/mL in water, with equal amounts of each enzyme component. 22.22 ul of a cocktail were added to the substrate and mixed by pipette for a final loading of 12 mg enzyme/g cellulose. The digest plates were centrifuged at 4000 rpm for 1 minute and 15 ul of the supernatant was transferred to 45 ul 200 mM NaCarbonate pH10 in a 384-well plate (“Stop Plate”). The digest plates were sealed and incubated at 37° C. Additional timepoints were taken at 22 and 70 hours.

The beta-glucosidase and GO assay steps are substantially described in Example 5. Conversion was calculated based on the theoretical cellulose value in the bagasse substrate.

| | Timepoints | | |
|---|---|---|---|
| enzymes | 0 | 22 | 70 |
| SEQ ID NO: 360 + SEQ ID NO: 358 + | 0 | 29.2% | 44.0% |
| SEQ ID NO: 500 | | | |
| SEQ ID NO: 360 + SEQ ID NO: 602 + | 0 | 35.6% | 49.1% |
| SEQ ID NO: 500 | | | |
| SEQ ID NO: 604 + SEQ ID NO: 358 + | 0 | 35.8% | 48.1% |
| SEQ ID NO: 500 | | | |
| SEQ ID NO: 604 + SEQ ID NO: 602 + | 0 | 38.6% | 50.9% |
| SEQ ID NO: 500 | | | |
| SEQ ID NO: 360 + SEQ ID NO: 358 | 0 | 15.5% | 30.0% |
| SEQ ID NO: 358 + SEQ ID NO: 602 | 0 | 20.2% | 34.1% |
| SEQ ID NO: 360 | 0 | 6.2% | 14.7% |
| SEQ ID NO: 604 | 0 | 11.2% | 21.1% |
| SEQ ID NO: 360 + SEQ ID NO: 500 | 0 | 25.9% | 34.1% |
| SEQ ID NO: 360 + SEQ ID NO: 602 | 0 | 16.8% | 28.8% |
| SEQ ID NO: 604 + SEQ ID NO: 500 | 0 | 32.7% | 46.6% |
| SEQ ID NO: 604 + SEQ ID NO: 602 | 0 | 21.0% | 32.2% |
| SEQ ID NO: 602 + SEQ ID NO: 500 | 0 | 13.0% | 24.7% |
| SEQ ID NO: 604 + SEQ ID NO: 358 | 0 | 3.9% | 8.1% |
| SEQ ID NO: 358 | 0 | 1.8% | 4.9% |
| SEQ ID NO: 602 | 0 | 4.2% | 8.0% |
| SEQ ID NO: 358 + SEQ ID NO: 500 | 0 | 15.2% | 19.3% |
| SEQ ID NO: 360 + SEQ ID NO: 604 | 0 | 9.0% | 19.9% |
| SEQ ID NO: 500 | 0 | 11.0% | 16.8% |

Ratio Optimization (D.O.E.):

Method

Steam exploded bagasse was resuspended in MES buffered minimal media (pH 5.6) at 1% solids. 160 ul of buffered substrate was added to each well in a 96-well plate, along with 2 metal BBs. Enzyme cocktails were prepared with volumes of each component dependent on desired enzyme ratio. 40 ul of a cocktail were added to the substrate and mixed by pipette for a final loading of 25 mg enzyme/g cellulose. The digest plates were centrifuged at 4000 rpm for 1 minute and 20 ul of the supernatant was transferred to 60 ul of 150 mM NaCarbonate pH10 in a 384-well plate (“Stop Plate”). The digest plates were sealed and incubated at 35° C. while shaking at 250 rpm. Additional timepoints were taken at 7, 26 and 50 hours. The B-glucosidase and GO assay steps are substantially as described in Example 5. Conversion was calculated based on the theoretical cellulose value in the bagasse substrate.

| | |
|---|---|
| SEQ ID NO: 360 + | |
| SEQ ID NO: 358 + | |
| SEQ ID NO: 90 | timepoints (hrs) |

| | enzyme ratio | 7 | 26 | 50 |
|---|---|---|---|---|
| 1 | 1:0:0 | 2.95% | 7.38% | 19.59% |
| 2 | 0:1:0 | 2.21% | 4.75% | 8.80% |
| 3 | 0:0:1 | 1.36% | 2.23% | 5.10% |
| 4 | 1:1:0 | 5.06% | 14.46% | 26.07% |
| 5 | 1:0:1 | 8.90% | 18.40% | 27.23% |
| 6 | 0:1:1 | −1.28% | 15.88% | 17.12% |
| 7 | 1:1:1 | 12.18% | 24.52% | 40.26% |
| 8 | 4:1:1 | 9.44% | 23.68% | 37.53% |
| 9 | 1:4:1 | 9.90% | 24.16% | 39.22% |
| 10 | 1:1:4 | 12.73% | 26.11% | 38.93% |

| | |
|---|---|
| SEQ ID NO: 360 + | |
| SEQ ID NO: 358 + | |
| SEQ ID NO: 500 | timepoints (hrs) |

| | enzyme ratio | 7 | 26 | 50 |
|---|---|---|---|---|
| 1 | 1:0:0 | 2.88% | 7.96% | 17.65% |
| 2 | 0:1:0 | 2.66% | 5.25% | 8.17% |
| 3 | 0:0:1 | 5.98% | 9.37% | 14.51% |
| 4 | 1:1:0 | 5.71% | 17.87% | 28.39% |
| 5 | 1:0:1 | 8.48% | 19.94% | 31.52% |
| 6 | 0:1:1 | −1.18% | 13.27% | 18.77% |
| 7 | 1:1:1 | 11.72% | 26.88% | 41.74% |
| 8 | 4:1:1 | 10.29% | 27.25% | 41.59% |
| 9 | 1:4:1 | 10.70% | 24.98% | 40.60% |
| 10 | 1:1:4 | 9.68% | 24.13% | 28.71% |

| | |
|---|---|
| SEQ ID NO: 360 + | |
| SEQ ID NO: 358 + | |
| SEQ ID NO: 502 | timepoints (hrs) |

| | enzyme ratio | 7 | 26 | 50 |
|---|---|---|---|---|
| 1 | 1:0:0 | 2.18% | 8.32% | 18.92% |
| 2 | 0:1:0 | 2.34% | 4.14% | 9.02% |
| 3 | 0:0:1 | 8.71% | 12.33% | 16.73% |
| 4 | 1:1:0 | 4.99% | 15.35% | 25.84% |
| 5 | 1:0:1 | 10.21% | 17.60% | 34.20% |
| 6 | 0:1:1 | −0.63% | 13.57% | 16.97% |
| 7 | 1:1:1 | 13.33% | 27.64% | 44.73% |
| 8 | 4:1:1 | 9.47% | 25.30% | 38.45% |
| 9 | 1:4:1 | 10.24% | 25.13% | 41.67% |
| 10 | 1:1:4 | 11.05% | 25.51% | 38.88% |

| | |
|---|---|
| SEQ ID NO: 360 + | |
| SEQ ID NO: 598 + | |
| SEQ ID NO: 90 | timepoints (hrs) |

| | enzyme ratio | 7 | 26 | 50 |
|---|---|---|---|---|
| 1 | 1:0:0 | 2.46% | 7.92% | 17.59% |
| 2 | 0:1:0 | 3.06% | 7.14% | 11.30% |
| 3 | 0:0:1 | 0.55% | 2.17% | 3.06% |
| 4 | 1:1:0 | 5.89% | 14.77% | 24.96% |
| 5 | 1:0:1 | 7.51% | 18.03% | 25.56% |
| 6 | 0:1:1 | −3.49% | 9.84% | 14.54% |
| 7 | 1:1:1 | 8.80% | 21.11% | 36.29% |
| 8 | 4:1:1 | 8.11% | 20.95% | 32.51% |
| 9 | 1:4:1 | 7.49% | 16.79% | 27.12% |
| 10 | 1:1:4 | 7.33% | 19.77% | 27.84% |

| | |
|---|---|
| SEQ ID NO: 360 + | |
| SEQ ID NO: 598 + | |
| SEQ ID NO: 500 | timepoints (hrs) |

| | enzyme ratio | 7 | 26 | 50 |
|---|---|---|---|---|
| 1 | 1:0:0 | 2.48% | 9.18% | 21.12% |
| 2 | 0:1:0 | 3.13% | 7.50% | 11.13% |
| 3 | 0:0:1 | 5.71% | 10.97% | 14.29% |
| 4 | 1:1:0 | 5.60% | 17.49% | 26.41% |
| 5 | 1:0:1 | 8.91% | 19.23% | 32.27% |
| 6 | 0:1:1 | −3.05% | 11.73% | 16.19% |
| 7 | 1:1:1 | 9.61% | 22.22% | 40.87% |
| 8 | 4:1:1 | 8.93% | 22.71% | 36.09% |
| 9 | 1:4:1 | 8.89% | 19.41% | 28.79% |
| 10 | 1:1:4 | 7.32% | 20.54% | 29.78% |

| | |
|---|---|
| SEQ ID NO: 360 + | |
| SEQ ID NO: 598 + | |
| SEQ ID NO: 502 | timepoints (hrs) |

| | enzyme ratio | 7 | 26 | 50 |
|---|---|---|---|---|
| 1 | 1:0:0 | 2.40% | 8.40% | 18.15% |
| 2 | 0:1:0 | 2.26% | 6.25% | 12.05% |
| 3 | 0:0:1 | 7.69% | 12.34% | 15.05% |
| 4 | 1:1:0 | 5.49% | 15.29% | 22.97% |
| 5 | 1:0:1 | 10.06% | 20.99% | 30.39% |
| 6 | 0:1:1 | −2.78% | 14.58% | 18.21% |
| 7 | 1:1:1 | 10.66% | 24.50% | 45.32% |
| 8 | 4:1:1 | 8.34% | 24.54% | 43.78% |
| 9 | 1:4:1 | 8.40% | 21.06% | 35.09% |
| 10 | 1:1:4 | 7.61% | 22.16% | 35.57% |

| | |
|---|---|
| SEQ ID NO: 600 + | |
| SEQ ID NO: 358 + | |
| SEQ ID NO: 90 | timepoints (hrs) |

| | enzyme ratio | 7 | 26 | 50 |
|---|---|---|---|---|
| 1 | 1:0:0 | 3.09% | 6.83% | 14.55% |
| 2 | 0:1:0 | 2.91% | 4.67% | 7.80% |
| 3 | 0:0:1 | 0.17% | 1.81% | 4.19% |
| 4 | 1:1:0 | 4.53% | 13.34% | 25.02% |
| 5 | 1:0:1 | 10.50% | 19.20% | 27.23% |
| 6 | 0:1:1 | 1.41% | 16.29% | 20.61% |
| 7 | 1:1:1 | 12.15% | 25.47% | 37.18% |
| 8 | 4:1:1 | 7.51% | 20.45% | 31.59% |
| 9 | 1:4:1 | 8.18% | 19.47% | 33.19% |
| 10 | 1:1:4 | 12.71% | 24.87% | 34.87% |

| | |
|---|---|
| SEQ ID NO: 600 + | |
| SEQ ID NO: 358 + | |
| SEQ ID NO: 500 | timepoints (hrs) |

| | enzyme ratio | 7 | 26 | 50 |
|---|---|---|---|---|
| 1 | 1:0:0 | 3.98% | 9.33% | 18.83% |
| 2 | 0:1:0 | 2.67% | 5.09% | 8.34% |
| 3 | 0:0:1 | 5.24% | 10.93% | 15.68% |
| 4 | 1:1:0 | 4.41% | 15.19% | 25.74% |
| 5 | 1:0:1 | 9.04% | 18.78% | 28.19% |
| 6 | 0:1:1 | −0.01% | 14.03% | 18.77% |
| 7 | 1:1:1 | 10.29% | 26.53% | 35.70% |
| 8 | 4:1:1 | 8.57% | 26.13% | 35.71% |
| 9 | 1:4:1 | 7.84% | 22.05% | 31.31% |
| 10 | 1:1:4 | 9.31% | 22.60% | 22.74% |

| | |
|---|---|
| SEQ ID NO: 600 + | |
| SEQ ID NO: 358 + | |
| SEQ ID NO: 502 | timepoints (hrs) |

| | enzyme ratio | 7 | 26 | 50 |
|---|---|---|---|---|
| 1 | 1:0:0 | 3.77% | 8.12% | 16.72% |
| 2 | 0:1:0 | 2.73% | 4.64% | 8.17% |
| 3 | 0:0:1 | 7.52% | 12.72% | 18.42% |
| 4 | 1:1:0 | 4.01% | 13.95% | 22.30% |
| 5 | 1:0:1 | 10.34% | 19.33% | 30.76% |
| 6 | 0:1:1 | −0.10% | 13.60% | 19.24% |
| 7 | 1:1:1 | 10.50% | 23.28% | 35.85% |
| 8 | 4:1:1 | 9.57% | 25.39% | 34.05% |
| 9 | 1:4:1 | 8.58% | 23.47% | 33.90% |
| 10 | 1:1:4 | 11.08% | 20.87% | 30.97% |

| | |
|---|---|
| SEQ ID NO: 600 + | |
| SEQ ID NO: 598 + | |
| SEQ ID NO: 90 | timepoints (hrs) |

| | enzyme ratio | 7 | 26 | 50 |
|---|---|---|---|---|
| 1 | 1:0:0 | 2.95% | 7.09% | 13.88% |
| 2 | 0:1:0 | 3.45% | 7.17% | 11.49% |
| 3 | 0:0:1 | 0.63% | 1.92% | 1.84% |
| 4 | 1:1:0 | 4.06% | 16.37% | 27.10% |
| 5 | 1:0:1 | 9.74% | 20.63% | 25.82% |
| 6 | 0:1:1 | −2.26% | 9.02% | 18.36% |
| 7 | 1:1:1 | 11.88% | 25.40% | 37.81% |
| 8 | 4:1:1 | 8.88% | 23.35% | 35.56% |
| 9 | 1:4:1 | 9.67% | 17.67% | 33.61% |
| 10 | 1:1:4 | 11.58% | 25.61% | 33.39% |

| | |
|---|---|
| SEQ ID NO: 600 + | |
| SEQ ID NO: 598 + | |
| SEQ ID NO: 500 | timepoints (hrs) |

| | enzyme ratio | 7 | 26 | 50 |
|---|---|---|---|---|
| 1 | 1:0:0 | 3.07% | 7.45% | 17.08% |
| 2 | 0:1:0 | 3.68% | 7.72% | 10.87% |
| 3 | 0:0:1 | 4.78% | 11.56% | 15.39% |
| 4 | 1:1:0 | 7.81% | 20.45% | 28.27% |
| 5 | 1:0:1 | 8.76% | 20.15% | 28.68% |
| 6 | 0:1:1 | −2.13% | 13.25% | 18.02% |
| 7 | 1:1:1 | 12.35% | 26.57% | 38.78% |
| 8 | 4:1:1 | 7.89% | 21.12% | 30.99% |
| 9 | 1:4:1 | 7.43% | 15.77% | 35.67% |
| 10 | 1:1:4 | 10.73% | 18.58% | 32.46% |

| | |
|---|---|
| SEQ ID NO: 600 + | |
| SEQ ID NO: 598 + | |
| SEQ ID NO: 502 | timepoints (hrs) |

| | enzyme ratio | 7 | 26 | 50 |
|---|---|---|---|---|
| 1 | 1:0:0 | 3.30% | 8.38% | 16.36% |
| 2 | 0:1:0 | 2.45% | 6.66% | 10.98% |
| 3 | 0:0:1 | 7.80% | 13.21% | 16.74% |
| 4 | 1:1:0 | 6.69% | 18.04% | 24.76% |
| 5 | 1:0:1 | 9.54% | 16.12% | 32.56% |
| 6 | 0:1:1 | −2.71% | 16.28% | 13.41% |
| 7 | 1:1:1 | 12.70% | 25.20% | 40.74% |
| 8 | 4:1:1 | 9.43% | 25.41% | 36.63% |
| 9 | 1:4:1 | 10.34% | 22.03% | 36.80% |
| 10 | 1:1:4 | 9.05% | 14.69% | 26.07% |

### Example 20

### Enzyme “Cocktails for Biomass Conversion

In one embodiment, the invention provides enzyme “cocktails” or mixtures (“cocktails” meaning mixtures of enzymes comprising at least one enzyme of this invention) to process, or “convert”, biomass, e.g., for making a biofuel such as bioethanol, biobutanol, biopropanol, biodiesel and the like. Enzyme “cocktails” or mixtures of the invention can be used to hydrolyze the major components of a lignocellulosic biomass, or any composition comprising cellulose and/or hemicellulose (lignocellulosic biomass also comprises lignin), e.g., seeds, grains, tubers, plant waste (such as a hay or straw, e.g., a rice straw or a wheat straw, or any the dry stalk of any cereal plant) or byproducts of food processing or industrial processing (e.g., stalks), corn (including cobs, stover, and the like), grasses (e.g., Indian grass, such as Sorghastrum nutans; or, switch grass, e.g., Panicum species, such as Panicum virgatum), wood (including wood chips, processing waste, such as wood waste), paper, pulp, recycled paper (e.g., newspaper); also including a monocot or a dicot, or a monocot corn, sugarcane or parts thereof (e.g., cane tops), rice, wheat, barley, switchgrass or Miscanthus; or a dicot oilseed crop, soy, canola, rapeseed, flax, cotton, palm oil, sugar beet, peanut, tree, poplar or lupine.

Enzyme “cocktails” or mixtures of the invention can include any combination of enzymes, e.g., ferulic acid esterases, arabinofuranosidases, alpha-glucuronidases, acetyl xylan esterases, xylosidases, xylanases, endoglucanases and beta-glucanases, etc., where in alternative embodiments at least one, or several or all of the enzymes of the “cocktail” or mixture is/are an enzyme of this invention.

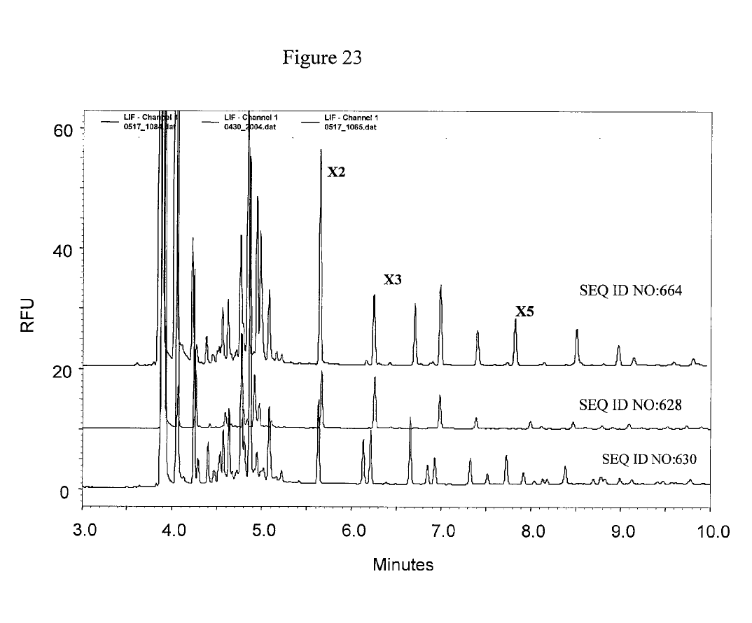

For example, FIG. 23 illustrates data showing the wheat arabinoxylan digest products (digest profiles) of three enzymes of the invention (the exemplary SEQ ID NO:664; SEQ ID NO:630; SEQ ID NO:628) that can be used in enzyme “cocktails” or mixtures of the invention; these three enzymes are xylanases initially derived from different Cochliobolus. Each enzyme was used to digest wheat arabinoxylan and the resulting products analyzed by capillary electrophoresis.

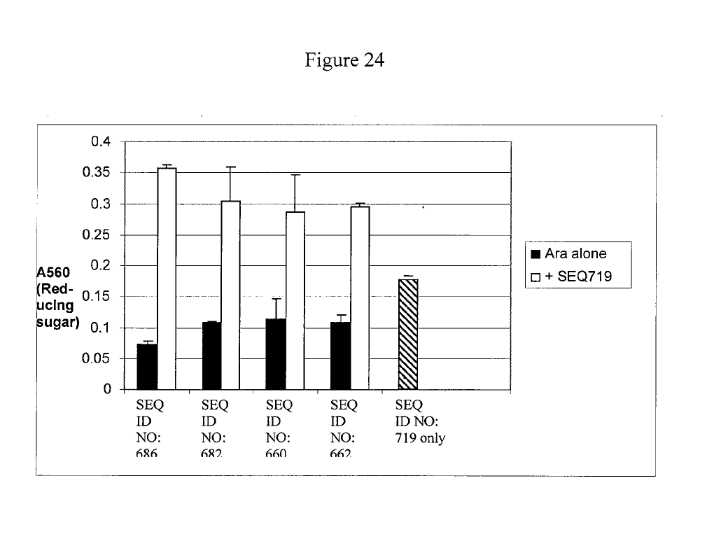

FIG. 24 is a graphic illustration of data showing how arabinofuranosidases of the invention (the exemplary SEQ ID NO:686; SEQ ID NO:682; SEQ ID NO:660; SEQ ID NO:662) synergize with xylanases of the invention to digest wheat arabinoxylan; and this figure also illustrates an exemplary “cocktail” or mixture of the invention. Exemplary arabinofuranosidases of the invention were used to digest wheat arabinoxylan with or without xylanase; e.g., the polypeptide SEQ ID NO:719. The amount of substrate digestion was measured with the BCA assay for reducing sugars.

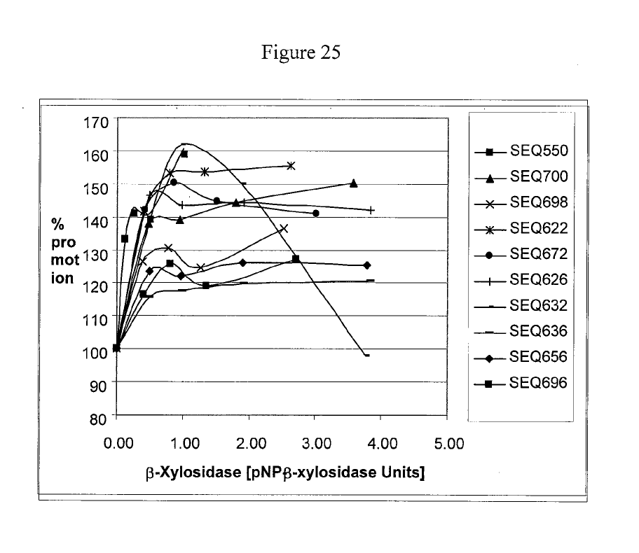

FIG. 25 is a graphic illustration of data showing a promotion effect of beta (B)-xylosidases of the invention SEQ ID NO:550; SEQ ID NO:700; SEQ ID NO:698; SEQ ID NO:622; SEQ ID NO:672; SEQ ID NO:626; SEQ ID NO:632; SEQ ID NO:636; SEQ ID NO:656; and, SEQ ID NO:696 (as indicated in the figure) over the exemplary SEQ ID NO:719 xylanase in a wheat arabinoxylan digest. Wheat arabinoxylan was digested with individual β-xylosidases in combination with xylanase SEQ ID NO:719; the BCA assay was used to quantify the reducing sugars produced. The % increase over the SEQ ID NO:719 xylanase alone is presented.

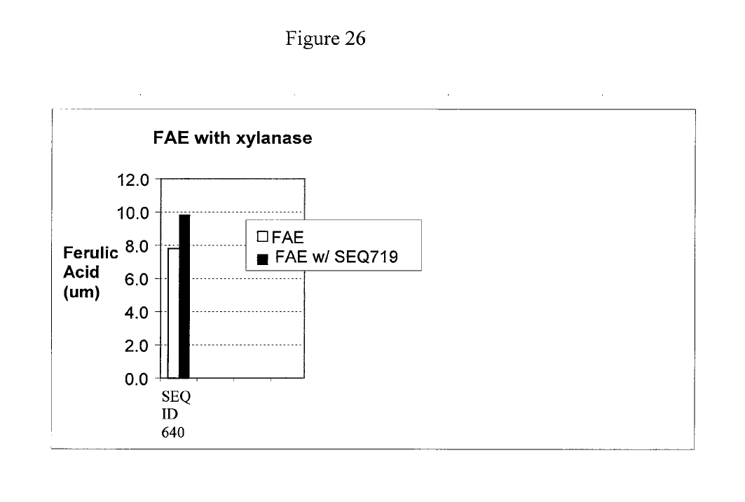

FIG. 26 is a graphic illustration of data showing a ferulic acid esterase (FAE) activity with corn seed fiber as a substrate using an exemplary enzyme of this invention. Corn seed fiber was digested with the ferulic acid esterase (FAE) SEQ ID NO:640 with or without xylanase SEQ ID NO:719 and the resulting ferulic acid produce was measured by HPLC. This mixture of the FAE SEQ ID NO:640 and the xylanase SEQ ID NO:719 is an exemplary mixture of this invention.

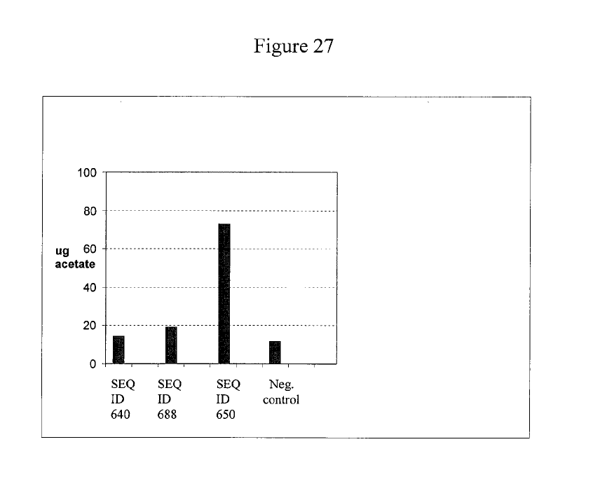

FIG. 27 is a graphic illustration of data showing from an activity assay with acetylated xylan as a substrate using the exemplary acetyl xylan esterases of this invention SEQ ID NO:640, SEQ ID NO:650 and SEQ ID NO:688. Acetate release from acetylated xylan was used to demonstrate acetyl xylan esterase activity. The figure shows esterase activity on 500 μg acetylated xylan reactions, pH 5, 24 hr incubation.

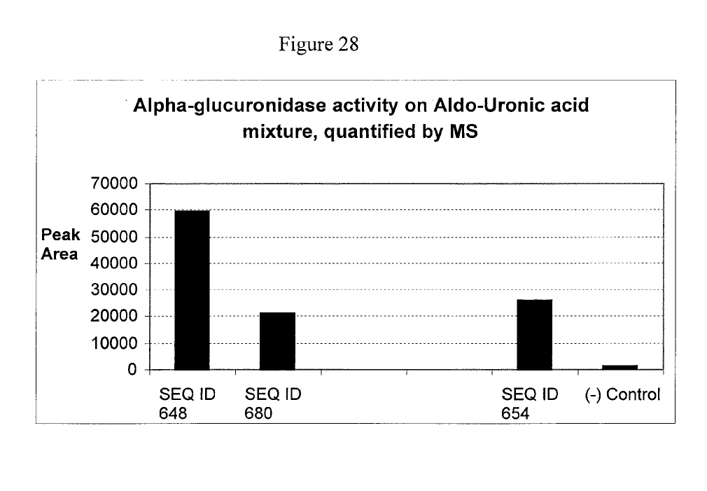

FIG. 28 is a graphic illustration of data showing an alpha (α)-glucuronidase activity assay with an aldo-uronic acid mixture as a substrate using the exemplary acetyl xylan esterases of this invention SEQ ID NO:648, SEQ ID NO:654 and SEQ ID NO:680. LC-MS was used to detect glucuronic acid release to demonstrate α-glucuronidase activity on an aldo-uronic acid mixture

A number of aspects of the invention have been described. Nevertheless, it will be understood that various modifications may be made without departing from the spirit and scope of the invention. Accordingly, other aspects are within the scope of the following claims.

1. An isolated, synthetic or recombinant nucleic acid comprising
(a) a nucleic acid sequence (polynucleotide) having at least 80%, 81%, 82%, 83%, 84%, 85%, 86%, 87%, 88%, 89%, 90%, 91%, 92%, 93%, 94%, 95%, 96%, 97%, 98%, 99%, or more or complete (100%) sequence identity to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, SEQ ID NO:169, SEQ ID NO:171, SEQ ID NO:173, SEQ ID NO:175, SEQ ID NO:177, SEQ ID NO:179, SEQ ID NO:181, SEQ ID NO:183, SEQ ID NO:185, SEQ ID NO:187, SEQ ID NO:189, SEQ ID NO:191, SEQ ID NO:193, SEQ ID NO:195, SEQ ID NO:197, SEQ ID NO:199, SEQ ID NO:201, SEQ ID NO:203, SEQ ID NO:205, SEQ ID NO:207, SEQ ID NO:209, SEQ ID NO:211, SEQ ID NO:213, SEQ ID NO:215, SEQ ID NO:217, SEQ ID NO:219, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:239, SEQ ID NO:241, SEQ ID NO:243, SEQ ID NO:245, SEQ ID NO:247, SEQ ID NO:249, SEQ ID NO:251, SEQ ID NO:253, SEQ ID NO:255, SEQ ID NO:257, SEQ ID NO:259, SEQ ID NO:261, SEQ ID NO:263, SEQ ID NO:265, SEQ ID NO:267, SEQ ID NO:269, SEQ ID NO:271, SEQ ID NO:273, SEQ ID NO:275, SEQ ID NO:277, SEQ ID NO:279, SEQ ID NO:281, SEQ ID NO:283, SEQ ID NO:285, SEQ ID NO:287, SEQ ID NO:289, SEQ ID NO:291, SEQ ID NO:293, SEQ ID NO:295, SEQ ID NO:297, SEQ ID NO:299, SEQ ID NO:301, SEQ ID NO:303, SEQ ID NO:305, SEQ ID NO:307, SEQ ID NO:309, SEQ ID NO:311, SEQ ID NO:313, SEQ ID NO:315, SEQ ID NO:317, SEQ ID NO:319, SEQ ID NO:321, SEQ ID NO:323, SEQ ID NO:325, SEQ ID NO:327, SEQ ID NO:329, SEQ ID NO:331, SEQ ID NO:333, SEQ ID NO:335, SEQ ID NO:337, SEQ ID NO:339, SEQ ID NO:341, SEQ ID NO:343, SEQ ID NO:345, SEQ ID NO:347, SEQ ID NO:349, SEQ ID NO:351, SEQ ID NO:353, SEQ ID NO:355, SEQ ID NO:357, SEQ ID NO:359, SEQ ID NO:361, SEQ ID NO:363, SEQ ID NO:365, SEQ ID NO:367, SEQ ID NO:369, SEQ ID NO:370, SEQ ID NO:372, SEQ ID NO:373, SEQ ID NO:375, SEQ ID NO:376, SEQ ID NO:378, SEQ ID NO:379, SEQ ID NO:381, SEQ ID NO:382, SEQ ID NO:384, SEQ ID NO:385, SEQ ID NO:387, SEQ ID NO:388, SEQ ID NO:390, SEQ ID NO:391, SEQ ID NO:393, SEQ ID NO:394, SEQ ID NO:396, SEQ ID NO:397, SEQ ID NO:399, SEQ ID NO:400, SEQ ID NO:402, SEQ ID NO:403, SEQ ID NO:405, SEQ ID NO:406, SEQ ID NO:408, SEQ ID NO:409, SEQ ID NO:411, SEQ ID NO:412, SEQ ID NO:414, SEQ ID NO:415, SEQ ID NO:417, SEQ ID NO:418, SEQ ID NO:420, SEQ ID NO:421, SEQ ID NO:423, SEQ ID NO:425, SEQ ID NO:427, SEQ ID NO:429, SEQ ID NO:431, SEQ ID NO:433, SEQ ID NO: 435, SEQ ID NO:437, SEQ ID NO:439, SEQ ID NO:441, SEQ ID NO:443, SEQ ID NO:445, SEQ ID NO:447, SEQ ID NO:449, SEQ ID NO:451, SEQ ID NO:453, SEQ ID NO:455, SEQ ID NO:457, SEQ ID NO:459, SEQ ID NO:461, SEQ ID NO:463, SEQ ID NO:465, SEQ ID NO:467, SEQ ID NO:469 and/or SEQ ID NO:471, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, all the odd numbered SEQ ID NOs: between SEQ ID NO:489 and SEQ ID NO:700, SEQ ID NO:707, SEQ ID NO:708, SEQ ID NO:709, SEQ ID NO:710, SEQ ID NO:711, SEQ ID NO:712, SEQ ID NO:713, SEQ ID NO:714, SEQ ID NO:715, SEQ ID NO:716, SEQ ID NO:717, SEQ ID NO:718, and/or SEQ ID NO:720, or nucleic acids encoding an enzymatically active subsequence (fragment) thereof,
wherein the nucleic acid (polynucleotide) encodes a polypeptide having a lignocellulosic activity, or encodes a polypeptide or peptide capable of generating an antibody that specifically binds to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:143, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:209, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, SEQ ID NO:260, SEQ ID NO:262, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, SEQ ID NO:360, SEQ ID NO:362, SEQ ID NO:364, SEQ ID NO:366, SEQ ID NO:368, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419, SEQ ID NO:422, SEQ ID NO:424, SEQ ID NO:426, SEQ ID NO:428, SEQ ID NO:430, SEQ ID NO:432, SEQ ID NO:434, SEQ ID NO: 436, SEQ ID NO:438, SEQ ID NO:440, SEQ ID NO:442, SEQ ID NO:444, SEQ ID NO:446, SEQ ID NO:448, SEQ ID NO:450, SEQ ID NO:452, SEQ ID NO:454, SEQ ID NO:456, SEQ ID NO:458, SEQ ID NO:460, SEQ ID NO:462, SEQ ID NO:464, SEQ ID NO:466, SEQ ID NO:468, SEQ ID NO:470 and/or SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, and/or enzymatically active subsequences (fragments) thereof, wherein optionally the lignocellulosic activity comprises a glycosyl transferase, a cellulase, a cellulolytic activity, an endoglucanase, a cellobiohydrolase, a beta-glucosidase, a xylanase, a mannanse, a β-xylosidase or an arabinofuranosidase activity,
and optionally the sequence identities are determined by analysis with a sequence comparison algorithm or by a visual inspection,
and optionally the sequence comparison algorithm comprises a BLAST version 2.2.2 algorithm where a filtering setting is set to blastall-p blastp-d “nr pataa” -F F, and all other options are set to default;
(b) a nucleic acid sequence (a polynucleotide) that hybridizes under stringent conditions to the complement of the nucleic acid of (a), wherein the nucleic acid encodes a polypeptide having a lignocellulosic activity, and optionally the lignocellulosic activity comprises a glycosyl transferase, a cellulase, a cellulolytic activity, an endoglucanase, a cellobiohydrolase, a beta-glucosidase, a xylanase, a mannanse, a β-xylosidase or an arabinofuranosidase activity,
and the stringent conditions comprise a wash step comprising a wash in 0.2×SSC at a temperature of about 65° C. for about 15 minutes;
(c) a nucleic acid sequence encoding a polypeptide having the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:143, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:209, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, SEQ ID NO:260, SEQ ID NO:262, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, SEQ ID NO:360, SEQ ID NO:362, SEQ ID NO:364, SEQ ID NO:366, SEQ ID NO:368, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419, SEQ ID NO:422, SEQ ID NO:424, SEQ ID NO:426, SEQ ID NO:428, SEQ ID NO:430, SEQ ID NO:432, SEQ ID NO:434, SEQ ID NO: 436, SEQ ID NO:438, SEQ ID NO:440, SEQ ID NO:442, SEQ ID NO:444, SEQ ID NO:446, SEQ ID NO:448, SEQ ID NO:450, SEQ ID NO:452, SEQ ID NO:454, SEQ ID NO:456, SEQ ID NO:458, SEQ ID NO:460, SEQ ID NO:462, SEQ ID NO:464, SEQ ID NO:466, SEQ ID NO:468, SEQ ID NO:470 and/or SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, or enzymatically active subsequences (fragments) thereof;
(d) the nucleic acid (polynucleotide) of (a), (b) or (c) and encoding a polypeptide having at least one conservative amino acid substitution and retaining its lignocellulosic activity,
wherein optionally the conservative amino acid substitution comprise substituting an amino acid with another amino acid of like characteristics, and optionally a conservative substitution comprises: replacement of an aliphatic amino acid with another aliphatic amino acid; replacement of a Serine with a Threonine or vice versa; replacement of an acidic residue with another acidic residue; replacement of a residue bearing an amide group with another residue bearing an amide group; exchange of a basic residue with another basic residue; or replacement of an aromatic residue with another aromatic residue;
(e) the nucleic acid (polynucleotide) of (a), (b), (c) or (d) encoding a polypeptide having a lignocellulosic activity but lacking a signal sequence, a prepro domain, a dockerin domain, and/or a carbohydrate binding module (CBM),
wherein optionally the carbohydrate binding module (CBM) comprises, or consists of, a cellulose binding module, a lignin binding module, a xylose binding module, a mannanase binding module, a xyloglucan-specific module and/or a arabinofuranosidase binding module;
(f) the nucleic acid (polynucleotide) of (a), (b), (c), (d) or (e) encoding a polypeptide having a lignocellulosic activity further comprising a heterologous sequence;
(g) the nucleic acid (polynucleotide) of (f), wherein the heterologous sequence comprises, or consists of a sequence encoding: (i) a heterologous signal sequence, a heterologous carbohydrate binding module, a heterologous dockerin domain, a heterologous catalytic domain (CD), or a combination thereof; (ii) the sequence of (ii), wherein the heterologous signal sequence, carbohydrate binding module or catalytic domain (CD) is derived from a heterologous lignocellulosic enzyme; or, (iii) a tag, an epitope, a targeting peptide, a cleavable sequence, a detectable moiety or an enzyme;
(h) the nucleic acid (polynucleotide) of (g), wherein the heterologous carbohydrate binding module (CBM) comprises, or consists of, a cellulose binding module, a lignin binding module, a xylose binding module, a mannanase binding module, a xyloglucan-specific module and/or a arabinofuranosidase binding module; or
(i) a nucleic acid sequence (polynucleotide) fully (completely) complementary to (a), (b), (c), (d), (e), (f), (g), or (h).

2-18. (canceled)

19. An isolated, synthetic or recombinant polypeptide comprising
(a) an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:143, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:209, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, SEQ ID NO:260, SEQ ID NO:262, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, SEQ ID NO:360, SEQ ID NO:362, SEQ ID NO:364, SEQ ID NO:366, SEQ ID NO:368, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419, SEQ ID NO:422, SEQ ID NO:424, SEQ ID NO:426, SEQ ID NO:428, SEQ ID NO:430, SEQ ID NO:432, SEQ ID NO:434, SEQ ID NO: 436, SEQ ID NO:438, SEQ ID NO:440, SEQ ID NO:442, SEQ ID NO:444, SEQ ID NO:446, SEQ ID NO:448, SEQ ID NO:450, SEQ ID NO:452, SEQ ID NO:454, SEQ ID NO:456, SEQ ID NO:458, SEQ ID NO:460, SEQ ID NO:462, SEQ ID NO:464, SEQ ID NO:466, SEQ ID NO:468, SEQ ID NO:470 and/or SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, or enzymatically active subsequences (fragments) thereof,
wherein polypeptide has a lignocellulosic activity, and optionally the lignocellulosic activity comprises a glycosyl transferase, a cellulase, a cellulolytic activity, an endoglucanase, a cellobiohydrolase, a beta-glucosidase, a xylanase, a mannanse, a β-xylosidase or an arabinofuranosidase activity, wherein optionally the sequence identities are determined by analysis with a sequence comparison algorithm or by a visual inspection, and optionally the sequence comparison algorithm is a BLAST version 2.2.2 algorithm where a filtering setting is set to blastall-p blastp-d “nr pataa” -F F, and all other options are set to default;
(b) an amino acid sequence encoded by the nucleic acid of claim 1, wherein the polypeptide has (i) a lignocellulosic activity, and optionally the lignocellulosic activity comprises a glycosyl transferase, a cellulase, a cellulolytic activity, an endoglucanase, a cellobiohydrolase, a beta-glucosidase, a xylanase, a mannanse, a β-xylosidase or an arabinofuranosidase activity, or, (ii) has immunogenic activity in that it is capable of generating an antibody that specifically binds to a polypeptide having the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:143, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170, SEQ ID NO:172, SEQ ID NO:174, SEQ ID NO:176, SEQ ID NO:178, SEQ ID NO:180, SEQ ID NO:182, SEQ ID NO:184, SEQ ID NO:186, SEQ ID NO:188, SEQ ID NO:190, SEQ ID NO:192, SEQ ID NO:194, SEQ ID NO:196, SEQ ID NO:198, SEQ ID NO:200, SEQ ID NO:202, SEQ ID NO:204, SEQ ID NO:206, SEQ ID NO:209, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:240, SEQ ID NO:242, SEQ ID NO:244, SEQ ID NO:246, SEQ ID NO:248, SEQ ID NO:250, SEQ ID NO:252, SEQ ID NO:254, SEQ ID NO:256, SEQ ID NO:258, SEQ ID NO:260, SEQ ID NO:262, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, SEQ ID NO:312, SEQ ID NO:314, SEQ ID NO:316, SEQ ID NO:318, SEQ ID NO:320, SEQ ID NO:322, SEQ ID NO:324, SEQ ID NO:326, SEQ ID NO:328, SEQ ID NO:330, SEQ ID NO:332, SEQ ID NO:334, SEQ ID NO:336, SEQ ID NO:338, SEQ ID NO:340, SEQ ID NO:342, SEQ ID NO:344, SEQ ID NO:346, SEQ ID NO:348, SEQ ID NO:350, SEQ ID NO:352, SEQ ID NO:354, SEQ ID NO:356, SEQ ID NO:358, SEQ ID NO:360, SEQ ID NO:362, SEQ ID NO:364, SEQ ID NO:366, SEQ ID NO:368, SEQ ID NO:371, SEQ ID NO:374, SEQ ID NO:377, SEQ ID NO:380, SEQ ID NO:383, SEQ ID NO:386, SEQ ID NO:389, SEQ ID NO:392, SEQ ID NO:395, SEQ ID NO:398, SEQ ID NO:401, SEQ ID NO:404, SEQ ID NO:407, SEQ ID NO:410, SEQ ID NO:413, SEQ ID NO:416, SEQ ID NO:419, SEQ ID NO:422, SEQ ID NO:424, SEQ ID NO:426, SEQ ID NO:428, SEQ ID NO:430, SEQ ID NO:432, SEQ ID NO:434, SEQ ID NO: 436, SEQ ID NO:438, SEQ ID NO:440, SEQ ID NO:442, SEQ ID NO:444, SEQ ID NO:446, SEQ ID NO:448, SEQ ID NO:450, SEQ ID NO:452, SEQ ID NO:454, SEQ ID NO:456, SEQ ID NO:458, SEQ ID NO:460, SEQ ID NO:462, SEQ ID NO:464, SEQ ID NO:466, SEQ ID NO:468, SEQ ID NO:470 and/or SEQ ID NO:472, SEQ ID NO:473, SEQ ID NO:474, SEQ ID NO:475, SEQ ID NO:476, SEQ ID NO:477, SEQ ID NO:478, SEQ ID NO:479, all the even numbered SEQ ID NOs: between SEQ ID NO:490 and SEQ ID NO:700, SEQ ID NO:719 and/or SEQ ID NO:721, and/or enzymatically active subsequences (fragments) thereof;
(c) the amino acid sequence of (a) or (b), and comprising at least one amino acid residue conservative substitution,
(d) the amino acid sequence of (c), wherein the conservative substitution comprises replacement of an aliphatic amino acid with another aliphatic amino acid; replacement of a serine with a threonine or vice versa; replacement of an acidic residue with another acidic residue; replacement of a residue bearing an amide group with another residue bearing an amide group; exchange of a basic residue with another basic residue; or, replacement of an aromatic residue with another aromatic residue, or a combination thereof,
and optionally the aliphatic residue comprises Alanine, Valine, Leucine, Isoleucine or a synthetic equivalent thereof; the acidic residue comprises Aspartic acid, Glutamic acid or a synthetic equivalent thereof; the residue comprising an amide group comprises Aspartic acid, Glutamic acid or a synthetic equivalent thereof; the basic residue comprises Lysine, Arginine or a synthetic equivalent thereof; or, the aromatic residue comprises Phenylalanine, Tyrosine or a synthetic equivalent thereof;
(e) the polypeptide of (a), (b), (c) or (d) having a lignocellulosic activity but lacking a signal sequence, a prepro domain, a dockerin domain, and/or a carbohydrate binding module (CBM),
wherein optionally the carbohydrate binding module (CBM) comprises, or consists of, a cellulose binding module, a lignin binding module, a xylose binding module, a mannanase binding module, a xyloglucan-specific module and/or a arabinofuranosidase binding module;
(f) the polypeptide of (a), (b), (c), (d) or (e) having a lignocellulosic activity further comprising a heterologous sequence;
(g) the polypeptide of (f), wherein the heterologous sequence comprises, or consists of: (i) a heterologous signal sequence, a heterologous carbohydrate binding module, a heterologous dockerin domain, a heterologous catalytic domain (CD), or a combination thereof; (ii) the sequence of (ii), wherein the heterologous signal sequence, carbohydrate binding module or catalytic domain (CD) is derived from a heterologous lignocellulosic enzyme; and/or, (iii) a tag, an epitope, a targeting peptide, a cleavable sequence, a detectable moiety or an enzyme; or
(h) the polypeptide of (g), wherein the heterologous carbohydrate binding module (CBM) comprises, or consists of, a cellulose binding module, a lignin binding module, a xylose binding module, a mannanase binding module, a xyloglucan-specific module and/or a arabinofuranosidase binding module; or (i) the polypeptide of any of (a) to (g), wherein the the polypeptide comprises at least one glycosylation site, and optionally the glycosylation site is an N-linked glycosylation, and optionally the polypeptide is glycosylated after being expressed in a P. Pastoris or a S. pombe.

20-101. (canceled)

102. A nucleic acid probe for identifying a nucleic acid encoding a polypeptide with a lignocellulosic activity, wherein the probe comprises
(a) at least 20, 30, 40, 50, 60, 75, 100, 125, 150, or 200 or more consecutive bases of the nucleic acid sequence (polynucleotide) of claim 1, wherein the probe identifies the nucleic acid by binding or hybridization;
(b) the probe of (a), wherein the probe comprises an oligonucleotide comprising at least about 10 to 50, about 20 to 60, about 30 to 70, about 40 to 80, about 60 to 100, or about 50 to 150 consecutive bases;
(c) the probe of (a) or (b) further comprising a detectable agent; or
(d) the probe of (c), wherein the detectable agent comprises a radioactive isotope, a fluorescent dye or an enzyme capable of catalyzing the formation of a detectable product.

103. An expression cassette, vector or cloning vehicle comprising a nucleic acid comprising the nucleic acid sequence of claim 1, wherein optionally the cloning vehicle comprises a viral vector, a plasmid, a phage, a phagemid, a cosmid, a fosmid, a bacteriophage or an artificial chromosome, and optionally the viral vector comprises an adenovirus vector, a retroviral vector or an adeno-associated viral vector, and optionally the cloning vehicle comprises a bacterial artificial chromosome (BAC), a plasmid, a bacteriophage P1-derived vector (PAC), a yeast artificial chromosome (YAC), or a mammalian artificial chromosome (MAC).

104. A transformed, infected, transformed or host cell comprising
(a) a nucleic acid comprising the nucleic acid sequence of claim 1;
(b) the cell of (a), wherein the cell is a bacterial cell, a mammalian cell, a fungal cell, a yeast cell, an insect cell or a plant cell;
(c) the plant cell of (b), wherein the plant cell is derived from a plant of the genera Anacardium, Arachis, Asparagus, Atropa, Avena, Brassica, Citrus, Citrullus, Capsicum, Carthamus, Cocos, Coffea, Cruciferae, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea; or
(d) the plant cell of (b), wherein the plant cell is derived from a corn plant, a sorghum plant, a potato plant, a tomato plant, a wheat plant, an oilseed plant, a rapeseed plant, a soybean plant, a rice plant, a barley plant, a grass, or a tobacco plant.

105. A transgenic non-human animal, plant, plant part or seed comprising the nucleic acid sequence of claim 1, wherein optionally the transgenic non-human animal is a mouse, rat, pig, cow or goat, wherein optionally the plant, plant part or seed is or is of a corn plant, a sorghum plant, a potato plant, a tomato plant, a wheat plant, an oilseed plant, a rapeseed plant, a soybean plant, a rice plant, a barley plant, a palm, a sunflower plant, a sesame plant, a rice, a peanut plant, a grass, or a tobacco plant, wherein optionally the plant, plant part or seed is or is of the genera Anacardium, Arachis, Asparagus, Atropa, Avena, Brassica, Citrus, Citrullus, Capsicum, Carthamus, Cocos, Coffea, Cruciferae, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.

106. A method of producing a recombinant polypeptide comprising:
(A) (a) providing a nucleic acid, wherein the nucleic acid comprises the nucleic acid sequence of claim 1; and
(b) expressing the nucleic acid of (a) under conditions that allow expression of the polypeptide, thereby producing a recombinant polypeptide; or
(B) the method of (A), wherein the method further comprises transforming a host cell with the nucleic acid of (a) followed by expressing the nucleic acid of (a), thereby producing a recombinant polypeptide in a transformed cell; or
(C) the method of (A) or (B), wherein the promoter is or comprises: a viral, bacterial, mammalian or plant promoter; or, a plant promoter; or, a potato, rice, corn, wheat, tobacco or barley promoter; or, a constitutive promoter or a CaMV35S promoter; or, an inducible promoter; or, a tissue-specific promoter or an environmentally regulated or a developmentally regulated promoter; or, a seed-specific, a leaf-specific, a root-specific, a stem-specific or an abscission-induced promoter; or, a seed preferred promoter, a maize gamma zein promoter or a maize ADP-gpp promoter.

107. A method of generating a variant of a nucleic acid encoding a polypeptide with a lignocellulosic activity comprising:
(I) (a) providing a template nucleic acid comprising the nucleic acid sequence of claim 1; and
(b) modifying, deleting or adding one or more nucleotides in the template sequence, or a combination thereof, to generate a variant of the template nucleic acid
wherein optionally the method further comprises expressing the variant nucleic acid to generate a variant polypeptide with a lignocellulosic activity,
and optionally the modifications, additions or deletions are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof
and optionally the method is iteratively repeated until a lignocellulosic enzyme having an altered or different activity or an altered or different stability from that of a polypeptide encoded by the template nucleic acid is produced; or
(II) the method of (I), wherein the polypeptide encoded by the variant nucleic acid (a) is thermotolerant, and retains some activity after being exposed to an elevated temperature; (b) has increased glycosylation as compared to the lignocellulosic enzyme encoded by a template nucleic acid; or, (c) has a lignocellulosic activity under a high temperature, wherein the lignocellulosic enzyme encoded by the template nucleic acid is not active under the high temperature.

108. A method for modifying codons in a nucleic acid encoding a lignocellulosic enzyme, the method comprising:
(a) providing a nucleic acid encoding a polypeptide with a lignocellulosic activity comprising the nucleic acid sequence of claim 1; and,
(b) identifying a codon in the nucleic acid of (a) and replacing it with a different codon encoding the same amino acid as the replaced codon, thereby modifying codons in a nucleic acid encoding a lignocellulosic enzyme.

109. A chimeric polypeptide comprising
(a) at least a first domain comprising a signal sequence (signal peptide (SP)) or leader sequence as set forth in the amino terminal residues 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, 1 to 40, 1 to 41, 1 to 42, 1 to 43 or 1 to 44, of (a) a polypeptide of claim 19, and at least a second domain comprising a heterologous polypeptide or peptide, wherein the heterologous polypeptide or peptide is not naturally associated with the signal peptide (SP) or leader sequence,
and optionally the heterologous polypeptide or peptide is not a lignocellulosic enzyme, and optionally the heterologous polypeptide or peptide is amino terminal to, carboxy terminal to or on both ends of the signal peptide (SP) or leader sequence;
(b) a first domain and at least a second domain, wherein the first domain comprises a polypeptide of claim 19, and the second domain comprises at least one heterologous or modified carbohydrate binding domain (CBM), at least one internally rearranged CBM, a heterologous or modified dockerin domain, a heterologous or modified prepro domain, or a heterologous or modified active site;
(c) the chimeric polypeptide of (b), wherein the heterologous or modified or internally rearranged CBM and comprises, or consists of, a CBM_{—}1, CBM_{—}2, CBM_{—}2a, CBM_{—}2b, CBM_{—}3, CBM_{—}3a, CBM_{—}3b, CBM_{—}3c, CBM_{—}4, CBM_{—}5, CBM_{—}5_{—}12, CBM_{—}6, CBM_{—}7, CBM_{—}8, CBM_{—}9, CBM_{—}10, CBM_{—}11, CBM_{—}12, CBM_{—}13, CBM_{—}14, CBM_{—}15, CBM_{—}16 or any of the CBMs from a CMB family of CBM_{—}1 to CBM_{—}48; a glycosyl hydrolase binding domain; a CBM as set forth in Table 5 or Table 6; or, any combination thereof;
(d) the chimeric polypeptide of (b) or (c), wherein the at least one carbohydrate binding domain (CBM) is a cellulose-binding module or a lignin-binding domain;
(e) the chimeric polypeptide of (a), (b), or (c), wherein the at least one CBM is positioned approximate to the polypeptide's catalytic domain;
(f) the chimeric polypeptide of (e), wherein the at least one CBM is positioned: approximate to the C-terminus of the polypeptide's catalytic domain, or, approximate to the N-terminus of the polypeptide's catalytic domain, or both; or
(g) the chimeric polypeptide of any of (a), (b), (c), (d), (e), or (f), wherein the chimeric polypeptide is a recombinant chimeric protein.

110. A composition or product of manufacture comprising
(a) a mixture (or “cocktail”) of lignocellulosic enzymes comprising: (i) at least one of each of a endoglucanase, cellobiohydrolase I (CBH I), cellobiohydrolase II (CBH II) and β-glucosidase; (ii) at least one of each of an xylanase, β-xylosidase and arabinofuranosidase; or, (iii) a combination of at least one of (i) or (ii); wherein the mixture of (a) comprises at least one enzyme of claim 19;
(b) a mixture (or “cocktail”) of hemicellulose- and cellulose-hydrolyzing enzymes comprising: (i) at least one of each of a endoglucanase, lignocellulosic enzyme, cellobiohydrolase I (CBH I), cellobiohydrolase II (CBH II), arabinofuranosidase and xylanase; (ii) the mixture of (i), wherein the glucose oxidase is a glucose oxidase-1 or β-glucosidase; or (iii) the mixture of (i) or (ii), wherein the glucose oxidase is a glucose oxidase-2 or β-xylosidase; wherein the mixture of (b) comprises at least one enzyme of claim 19;
(c) a mixture (or “cocktail”) of hemicellulose- and cellulose-hydrolyzing enzymes comprising: at least one of each of a endoglucanase; a cellobiohydrolase I (CBH I); a cellobiohydrolase II (CBH II); an arabinofuranosidase; a xylanase; a glucose oxidase-1 (a β-glucosidase); and, a glucose oxidase-2 or β-xylosidase; wherein the mixture of (c) comprises at least one enzyme of claim 19;
(d) a mixture (or “cocktail”) of enzymes comprising: (1) an endoglucanase which cleaves internal β-1,4 linkages resulting in shorter glucooligosaccharides, (2) a cellobiohydrolase which acts in an “exo” manner processively releasing cellobiose units (β-1,4 glucose-glucose disaccharide), and (3) a β-glucosidase for releasing glucose monomer from short cellooligosaccharides (e.g. cellobiose); wherein the mixture of (d) comprises at least one enzyme of claim 19;
(e) a mixture (or “cocktail”) of enzymes comprising: SEQ ID NO:34, SEQ ID NO:360, SEQ ID NO:358, and SEQ ID NO:371; or, SEQ ID NO:358, SEQ ID NO:360, SEQ ID NO:168; or, SEQ ID NO:34, SEQ ID NO:360, SEQ ID NO:214; or, SEQ ID NO:360, SEQ ID NO:90, SEQ ID NO:358;
(f) a mixture (or “cocktail”) of enzymes comprising a combination of enzymes as set forth in Table 4;
(g) the composition or product of manufacture of any of (a) to (f), wherein the endoglucanase comprises SEQ ID NO:4, the cellobiohydrolase I comprises SEQ ID NO:16, SEQ ID NO:30 or SEQ ID NO:356, the cellobiohydrolase II comprises SEQ ID NO:282, the β-glucosidase comprises SEQ ID NO:124, the xylanase comprises SEQ ID NO:262, or any combination thereof, or
(h) the composition or product of manufacture of any of (a) to (g), wherein at least one enzyme comprises an additional carbohydrate binding domain (CBM).

111. A mixture or cocktail of enzymes comprising
(a) a polypeptide of claim 19 or a chimeric polypeptide of claim 109;
(b) a combination of enzymes as set forth in Table 4;
(c) at least one of each of a endoglucanase, cellobiohydrolase I (CBH I), cellobiohydrolase II (CBH II) and β-glucosidase; (ii) at least one of each of an xylanase, β-xylosidase and arabinofuranosidase; or, (iii) a combination of at least one of (i) or (ii); wherein the mixture comprises at least one enzyme of claim 19;
(d) a mixture (or “cocktail”) of enzymes comprising hemicellulose- and cellulose-hydrolyzing enzymes, wherein the cellulose-hydrolyzing enzymes comprise at least one glucose oxidase, endoglucanase, cellobiohydrolase I, cellobiohydrolase II and β-glucosidase; and the hemicellulose-hydrolyzing enzymes comprise at least one xylanase, β-xylosidase and arabinofuranosidase;
(e) at least one hemicellulose- and/or cellulose-hydrolyzing enzyme comprising: (i) at least one of each of a endoglucanase, glucose oxidase, cellobiohydrolase I (CBH I), cellobiohydrolase II (CBH II), arabinofuranosidase and xylanase; (ii) the mixture of (i), wherein the glucose oxidase is a glucose oxidase-1 or β-glucosidase; and/or (iii) the mixture of (i) or (ii), wherein the glucose oxidase is a glucose oxidase-2 or β-xylosidase; wherein the mixture comprises at least one enzyme of claim 19;
(f) at least one hemicellulose- and/or cellulose-hydrolyzing enzyme comprising: at least one of each of a endoglucanase; a cellobiohydrolase I (CBH I); a cellobiohydrolase II (CBH II); an arabinofuranosidase; a xylanase; a glucose oxidase-1 (a β-glucosidase); and/or, a glucose oxidase-2 or β-xylosidase; wherein the mixture of (c) comprises at least one enzyme of claim 19;
(g) at least one (1) endoglucanase which cleaves internal β-1,4 linkages resulting in shorter glucooligosaccharides, (2) cellobiohydrolase which acts in an “exo” manner processively releasing cellobiose units (β-1,4 glucose-glucose disaccharide), and/or (3) β-glucosidase for releasing glucose monomer from short cellooligosaccharides (e.g. cellobiose); wherein the mixture of (d) comprises at least one enzyme of claim 19; or
(h) the enzyme combination SEQ ID NO:34, SEQ ID NO:360, SEQ ID NO:358, and SEQ ID NO:371; or, SEQ ID NO:358, SEQ ID NO:360, SEQ ID NO:168; or, SEQ ID NO:34, SEQ ID NO:360, SEQ ID NO:214; or, SEQ ID NO:360, SEQ ID NO:90, SEQ ID NO:358.

112. A composition comprising a polypeptide of claim 19, a chimeric polypeptide of claim 109, a composition or product of manufacture of claim 110, or a mixture or cocktail of enzymes of claim 111, wherein optionally the composition is a pharmaceutical composition, a detergent composition, a contact lens solution, a waste treatment composition, a disinfectant, biodefense or bio-detoxifying agent, or wherein optionally the composition is a fuel, wherein optionally the composition is an alcohol, wherein optionally the alcohol is ethanol, or wherein optionally the composition is a biomass or a biomass material, a paper, paper waste, recycled paper product, paper pulp, paper product, wood, wood product, wood pulp, wood waste, textile, fabric, yarn, or fiber, or wherein optionally the composition is a cellulose- or a cellulose derivative-composition, or wherein optionally the composition is a beverage, a food, a feed, a food or feed supplement, a dairy product or a nutritional supplement, a dietary supplement, an edible enzyme delivery matrix or pellet, or wherein optionally the food is a dough, a bread or a baked product, or wherein optionally the biomass material is derived from an agricultural crop, or the biomass material is a byproduct of a food or a feed production, or the biomass material is a waste product, or the biomass material is a plant residue or a waste paper or waste paper product, or the biomass material comprises a plant residue, and optionally the plant residue comprises stems, leaves, hulls, husks, corn cobs, corn stover, straw, wood, wood chips, wood pulp and/or sawdust, and optionally the paper waste comprises discarded or used photocopy paper, computer printer paper, notebook paper, notepad paper, typewriter paper, newspapers, magazines, cardboard and paper-based packaging materials.

113. A method for hydrolyzing, breaking up or disrupting a cellooligsaccharide, an arabinoxylan oligomer, or a lignocellulose-, lignin-, xylan-, glucan- or cellulose-comprising composition comprising:
(A)(a) providing a polypeptide of claim 19, a chimeric polypeptide of claim 109, a composition or product of manufacture of claim 110, or a mixture or cocktail of enzymes of claim 111;
(b) providing a composition comprising a lignocellulose, lignin, xylan, cellulose and/or glucan; and
(c) contacting the polypeptide of (a) with the composition of (b) under conditions wherein the lignocellulosic enzyme hydrolyzes, breaks up or disrupts the lignin-, xylan-, cellooligsaccharide, arabinoxylan oligomer, or glucan- or cellulose-comprising composition;
(B) the method of (A), wherein the composition comprises a plant cell, a bacterial cell, a yeast cell, an insect cell, or an animal cell,
(C) the method of (A) or (B), wherein the polypeptide has glycosyl hydrolase, endoglucanase, cellobiohydrolase, beta-glucosidase, xylanase, mannanase, β-xylosidase and/or arabinofuranosidase activity;
(D) the method of (A), (B) or (C), wherein the polypeptide of (A)(a) is a recombinant polypeptide;
(E) the method of (D), wherein the recombinant polypeptide is produced as a heterologous recombinant polypeptide within the lignocellulose-, xylan-, lignin-, glucan- or cellulose-comprising composition to be hydrolyzed;
(F) the method of (D), wherein the recombinant polypeptide is produced by expression of a heterologous polynucleotide encoding the recombinant polypeptide in a bacterium, a yeast, a plant, an insect, a fungus and an animal, and optionally the organism is selected from the group consisting of an S. pombe, S. cerevisiae, Pichia pastoris, E. coli, Streptomyces sp., Bacillus sp. or a Lactobacillus sp.; or
(G) the methods of (A) to (F), wherein the lignocellulose-, lignin-, xylan-, glucan- or cellulose-comprising composition comprises: a monocot or dicot plant or plant product; or, a monocot corn, sugarcane, rice, wheat, barley, switchgrass or Miscanthus; or a dicot oilseed crop, soy, canola, rapeseed, flax, cotton, palm oil, sugar beet, peanut, tree, poplar or lupine.

114. A method for making a fuel comprising
(A) contacting a composition comprising a cellooligsaccharide, an arabinoxylan oligomer, a lignin, a lignocellulose, a xylan, a glucan, a cellulose or a fermentable sugar with the polypeptide of claim 19, chimeric polypeptide of claim 109, a composition or product of manufacture of claim 110, or a mixture of cocktail of enzymes of claim 111;
(B) the method of (A), wherein the composition comprising the cellooligsaccharide, arabinoxylan oligomer, lignin, lignocellulose, xylan, glucan, cellulose or fermentable sugar comprises a plant, plant product or plant derivative;
(C) the method of (A) or (B), wherein the plant or plant product comprises cane sugar plants or plant products, beets or sugarbeets, wheat, corn, soybeans, potato, rice or barley;
(D) the method of (C), wherein the plant is a monocot or dicot, or the plant is a monocot corn, sugarcane, rice, wheat, barley, switchgrass or Miscanthus; or the plant is a dicot oilseed crop, soy, canola, rapeseed, flax, cotton, palm oil, sugar beet, peanut, tree, poplar or lupine;
(E) the method of (A), (B), (C) or (D), wherein the polypeptide has activity comprising cellulase, endoglucanase, cellobiohydrolase, beta-glucosidase, xylanase, mannanase, β-xylosidase and/or arabinofuranosidase activity, wherein optionally the composition further comprises a glucose oxidase, a glucose oxidase-1 (a β-glucosidase) or a glucose oxidase-2 (a β-xylosidase); or
(F) the method of (A), (B), (C), (D) or (E), further comprising processing and/or formulating the fuel as a liquid and/or a gas, wherein optionally the fuel comprises a biofuel and/or a synthetic fuel, or the fuel comprises bioethanol, biomethanol, biopropanol and/or, bio-butanol; and/or a gasoline-ethanol, -methanol, -butanol and/or -propanol mix.

115. A method for processing a biomass material comprising contacting a biomass material with the polypeptide of claim 19, a chimeric polypeptide of claim 109, a mixture or cocktail of enzymes of claim 110, or a composition or product of manufacture of claim 111,
wherein optionally the biomass material is derived from an agricultural crop, is a byproduct of a food or a feed production, is a lignocellulosic waste product, or is a plant material, plant byproduct of a process or plant residue or a waste paper or waste paper product,
and optionally the plant material or plant residue comprises sugar cane bagasse, stems, leaves, hulls, husks, corn or corn cobs, corn stover, hay, straw, wood, wood chips, wood pulp, paper waste, wood waste and sawdust,
and optionally the paper waste comprises discarded or used photocopy paper, computer printer paper, notebook paper, notepad paper, typewriter paper, newspapers, magazines, cardboard and paper-based packaging materials,
and optionally further processing the biomass material to generate a bioalcohol, a bioethanol, biomethanol, biobutanol or biopropanol.

116. An isolated, synthetic and/or recombinant carbohydrate binding domain-module (CBM) comprising, or consisting of
(a) a carbohydrate binding domain-module (CBM) motif comprising, or consisting of, a subsequence of the polypeptide of claim 19, wherein the carbohydrate binding domain-module (CBM) comprises or consists of a CBM_{—}1, CBM_{—}2, CBM_{—}2a, CBM_{—}2b, CBM_{—}3, CBM_{—}3a, CBM_{—}3b, CBM_{—}3c, CBM_{—}4, CBM_{—}5, CBM_{—}5_{—12,} CBM_{—}6, CBM_{—}7, CBM_{—}8, CBM_{—}9, CBM_{—}10, CBM_{—}11, CBM_{—}12, CBM_{—}13, CBM_{—}14, CBM_{—}15, CBM_{—}16 or any of the CBMs from a CMB family of CBM_{—}1 to CBM_{—}48;
(b) at least one carbohydrate binding domain-module (CBM) as set forth in Table 5, and the Sequence Listing;
(c) at least one carbohydrate binding domain-module (CBM) as set forth in Table 6, and the Sequence Listing; or
(d) a combination thereof.

117. A method of treating or modifying a composition comprising contacting a composition with a polypeptide as set forth in claim 19, a chimeric polypeptide of claim 109, a composition or product of manufacture of claim 110, or a mixture or cocktail of enzymes of claim 111, wherein optionally the composition is a pharmaceutical composition, a detergent composition, a contact lens solution, a waste treatment composition, a disinfectant, biodefense or bio-detoxifying agent, or wherein optionally the composition is a fuel, wherein optionally the composition is an alcohol, wherein optionally the alcohol is ethanol, or wherein optionally the composition is a biomass or a biomass material, a paper, paper waste, recycled paper product, paper pulp, paper product, wood, wood product, wood pulp, wood waste, textile, fabric, yarn, or fiber, or wherein optionally the composition is a cellulose- or a cellulose derivative-composition, or wherein optionally the composition is a beverage, a food, a feed, a food or feed supplement, a dairy product or a nutritional supplement, a dietary supplement, an edible enzyme delivery matrix or pellet, or wherein optionally the food is a dough, a bread or a baked product, or wherein optionally the biomass material is derived from an agricultural crop, or the biomass material is a byproduct of a food or a feed production, or the biomass material is a waste product, or the biomass material is a plant residue or a waste paper or waste paper product, or the biomass material comprises a plant residue, and optionally the plant residue comprises stems, leaves, hulls, husks, corn cobs, corn stover, straw, wood, wood chips, wood pulp and/or sawdust, and optionally the paper waste comprises discarded or used photocopy paper, computer printer paper, notebook paper, notepad paper, typewriter paper, newspapers, magazines, cardboard and paper-based packaging materials.

118. An immobilized polypeptide or enzyme, or an immobilized nucleic acid, wherein the polypeptide comprises the sequence of claim 19, or the nucleic acid comprises the nucleic acid sequence of claim 1, wherein optionally the polypeptide or nucleic acid is immobilized on a cell, a metal, a resin, a polymer, a ceramic, a glass, a microelectrode, a graphitic particle, a bead, a gel, a plate, an array or a capillary tube.

119. An isolated, synthetic or recombinant antibody that specifically binds to the polypeptide of claim 19, wherein optionally the antibody is a monoclonal or a polyclonal antibody.

120. A method of isolating or recovering a nucleic acid encoding a polypeptide with a lignocellulosic activity from a sample comprising:
(a) providing a polynucleotide probe comprising, or consisting of, the nucleic acid sequence of claim 1, or the probe of claim 102;
(b) isolating a nucleic acid from the sample or treating the sample such that nucleic acid in the sample is accessible for hybridization to the polynucleotide probe of (a);
(c) combining the isolated nucleic acid or the treated sample of (b) with the polynucleotide probe of (a); and
(d) isolating a nucleic acid that specifically hybridizes with the polynucleotide probe of (a), thereby isolating or recovering a nucleic acid encoding a polypeptide with a lignocellulosic activity from an sample;
wherein optionally the sample is an environmental sample, or optionally the sample comprises a water sample, a liquid sample, a soil sample, an air sample or a biological sample, and optionally the biological sample is derived from a bacterial cell, a protozoan cell, an insect cell, a yeast cell, a plant cell, a fungal cell or a mammalian cell.
